(19) United States
(12) Patent Application Publication
(10) Pub. No.: US 2025/0313821 A1
(43) Pub. Date: Oct. 9, 2025

(54) EVOLVED CYTOSINE DEAMINASES AND METHODS OF EDITING DNA USING SAME

(71) Applicants: The Broad Institute, Inc., Cambridge, MA (US); President and Fellows of Harvard College, Cambridge, MA (US)

(72) Inventors: David R. Liu, Cambridge, MA (US); Monica Neugebauer, Cambridge, MA (US); Emily Zhang, Cambridge, MA (US)

(73) Assignees: The Broad Institute, Inc., Cambridge, MA (US); President and Fellows of Harvard College, Cambridge, MA (US)

(21) Appl. No.: 19/054,409

(22) Filed: Feb. 14, 2025

Related U.S. Application Data
(63) Continuation of application No. PCT/US2023/072257, filed on Aug. 15, 2023.
(60) Provisional application No. 63/398,483, filed on Aug. 16, 2022.
(60) Provisional application No. 63/380,523, filed on Oct. 21, 2022.

(51) Int. Cl. C12N 9/78; C12N 9/22; C12N 15/11; C12N 15/86

(52) U.S. Cl. CPC C12N 9/78; C12N 9/226; C12N 15/111; C12N 15/86; C07K 2319/80; C12N 2310/20; C12N 2750/14143; C12Y 305/04004; C12Y 305/04005

(57) ABSTRACT

The present disclosure generally relates to evolved cytidine deaminases derived from cytidine deaminases, and methods of editing DNA using the same. In some aspects, the disclosure describes the directed evolution of a TadA-derived adenosine deaminase (TadA-CD) to perform cytidine deamination. In some embodiments, the TadA-CDs comprise a plurality of mutations compared to the parent TadA variant. In some embodiments, the TadA-CD is fused to a programmable DNA binding protein. Other aspects of the disclosure generally relate to a cytosine base editor (CBE) comprising a programmable DNA binding protein and the TadA-CD. In some embodiments, the disclosed cytosine base editor has improved efficiencies of conversion and reduced off-target editing frequencies compared to naturally-occurring CBEs. Also provided are polynucleotides, vectors, and kits useful for the generation and delivery of the CBEs. Cells containing such vectors and CBEs are also provided. Further provided are methods of treatment comprising administering the CBEs.

### RELATED APPLICATIONS

This application claims priority under 35 U.S.C. §§ 120 and 365(c) to International PCT Application, PCT/US2023/072257, filed Aug. 15, 2023, which claims priority under 35 U.S.C. § 119(e) to U.S. Provisional Application, U.S. Ser. No. 63/398,483, filed on Aug. 16, 2022, and to U.S. Provisional Application, U.S. Ser. No. 63/380,523, filed on Oct. 21, 2022, each of which is incorporated herein by reference.

### GOVERNMENT SUPPORT

This invention was made with government support under grant numbers RM1HG009490, R01EB027793, R01EB031172, R35GM118062, R01HL156647, U19NS132304, U19NS132315, and U01AI142756, awarded by the National Institutes of Health. The government has certain rights in the invention.

### REFERENCE TO AN ELECTRONIC SEQUENCE LISTING

The content of the electronic sequence listing (B119570170US02-SEQ-GJM.xml; Size: 457,018 bytes; and Date of Creation: Feb. 13, 2025) is herein incorporated by reference in its entirety.

### BACKGROUND OF INVENTION

Base editors (BEs) are useful tools for performing in vivo forward genetic mutagenesis screens and have the potential to correct pathogenic point mutations by enabling precise installation of target point mutations in genomic DNA. BEs comprise fusions between a Cas protein and a base-modification enzyme (e.g., a deaminase). Cytosine base editors (CBEs) convert a C·G base pair to a T·A base pair, and adenine base editors (ABEs) convert an A·T base pair to a G·C base pair. Collectively, CBEs and ABEs can mediate all four possible transition mutations (e.g., C to T, A to G, T to C, and G to A). Reference is made to International Patent Application No.: PCT/US2017/045381, published Feb. 8, 2018, International Patent Application No.: PCT/US2018/056146, which published as WO 2019/079347 on Apr. 25, 2019, Koblan et al., Nat Biotechnol (2018) and Gaudelli et al., Nature 551, 464-471 (2017).

Highly active cytidine deaminases that natively modify DNA, such as APOBEC family enzymes, can deaminate transiently exposed single-stranded DNA segments beyond those in the R-loop generated by a Cas protein domain of a CBE, leading to low-level but widespread Cas-independent modification of the genome^{3-15,19}. Likewise, high-activity cytidine deaminases that can potently engage RNA can also mediate undesired RNA deamination that is not dependent on guide RNA hybridization²¹. The significant Cas-independent off-target DNA and RNA editing observed in editing with existing CBEs could limit the use of those CBEs in applications for which off-target editing should be minimized¹⁵. Existing CBEs include BE3, which comprises the structure NH₂-[NLS]-[rAPOBEC1 deaminase]-[Cas9 nickase (D10A)]-[UGI domain]-[NLS]-COOH; BE4, which comprises the structure NH₂-[NLS]-[rAPOBEC1 deaminase]-[Cas9 nickase (D10A)]-[UGI domain]-[UGI domain]-[NLS]-COOH; and BE4max, which is a version of BE4 for which the codons of the base editor-encoding construct has been codon-optimized for expression in human cells. Cas-independent off-target effects arise from stochastic associations of base editors with DNA sites due to an intrinsic affinity of an overexpressed base editor for DNA. Cas-independent off-target DNA editing has been found to be undetected or much less frequent for several TadA*-based ABEs¹³, although low-level RNA deamination can be detected from overexpression of some ABEs^{8,9,34}.

There is a need in the art for novel cytidine deaminases and cytosine base editors that maintain high-on target activity while exhibiting lower Cas-independent off-targeting editing. There is also a need in the art for CBEs of smaller sizes, for instance, sizes small enough to be encoded by a single adeno-associated viral (AAV) vector (e.g., packing capacity of ˜4.7 kb).

### SUMMARY OF THE INVENTION

The present disclosure provides the first directed evolution of a deaminase to selectively deaminate a different base. The present disclosure provides variants of adenosine deaminases that have been engineered to preferentially deaminate cytidine in DNA. Accordingly, the present disclosure provides cytidine deaminases that are variants of adenosine deaminases (e.g., wild-type or engineered tRNA adenosine deaminases (TadAs)). The present disclosure provides cytosine base editors that comprise a deaminase variant domain that preferentially deaminates cytidine in DNA and a nucleic acid programmable binding protein (napDNAbp) domain, wherein the adenosine deaminase variants are able to deaminate cytidines in nucleic acid molecules to a similar or the same degree as existing cytidine deaminases. In some aspects, the disclosure provides size-minimized deaminase variants that provide the base editor with reduced off-target effects relative to, while maintaining the high editing efficiencies of, existing cytosine base editors (CBEs). In some aspects, the disclosure provides base editors, complexes, nucleic acids, vectors, cells, compositions, methods, kits, and uses that utilize the deaminases and base editors provided herein.

This disclosure is based, at least in part, on the hypothesis that adenosine deaminases could be further evolved to recognize cytosine as a substrate, and this evolution may result in a new class of highly selective cytidine deaminases and CBEs with high editing efficiencies and lower off-target Cas-independent DNA and RNA editing (compared to naturally occurring cytidine deaminases). Wild-type TadA is evolutionarily related to cytidine deaminases. Further, low levels of cytidine deamination have been reported in evolved ABE variants^{11,31,32}. Also, mutagenesis of TadA7.10 (TadA-7.10 P48R) was shown to disrupt adenosine selectivity and increase cytidine deamination in 5′-TC contexts at protospacer position 6 in the editing window (counting the SpCas9 protospacer adjacent motif, PAM, as positions 21-23)³², although adenosine deamination is still preferred at other contexts and positions. Lastly, adenosine deaminases acting on RNA (ADARs) have been evolved to perform both cytidine and adenosine deamination in RNA³³.

The present disclosure generally relates to base editors (BEs) for gene editing. Base editors reported to date comprise, inter alia, a programmable DNA-binding protein domain (e.g., Cas9) fused to a deaminase (e.g., “base” modification domain). In some cases, BEs may also include additional domains that alter cellular DNA repair processes to increase the efficiency, incorporation, and/or stability of the resulting single-nucleotide change. The programmable DNA-binding domain directs the deaminase to directly convert one base to another at a guide RNA-programmed target site. Two primary classes of BEs have been developed to date: cytidine BEs (CBEs), which convert C·G to T·A, and adenine BEs (ABEs), which convert A·T to G·C. Collectively, CBEs and ABEs enable the correction of all four types of transition mutations (C to T, G to A, A to G, and T to C). As half of known disease-associated gene variants are point mutations, and transition mutations account for ˜60% of known pathogenic point mutations, BEs are being widely used to study and treat genetic diseases in a variety of cell types and organisms, including animal models of human genetic diseases.

CBEs and ABEs may include any programmable DNA binding domain known to one of skill in the art. CBEs further comprises deaminases configured to deaminate cytidine; whereas ABEs comprise deaminases configured to deaminate adenosine. Without wishing to be bound by any particular theory, it is generally believed that current CBEs comprise naturally occurring deaminases, or variants thereof, that are configured to deaminate cytidine to uracil. On the contrary, ABEs comprise a tRNA specific adenosine deaminase that has been evolved (e.g., mutated using laboratory techniques such as PACE and PANCE) to accept DNA substrates, such as those described in International Patent Application No. PCT/US2021/016827, filed Feb. 5, 2021, incorporated herein by reference, to enable A·T to G·C editing. All reported ABEs to date^{4,8-10}, including those already in clinical trials² or cleared for clinical trials¹, use TadA7.10 or evolved or engineered variants of this deaminase. TadA7.10 is the adenosine deaminase of the state-of-the-art ABE, ABE7.10, which is disclosed in International Publication No. WO 2018/027078, published Aug. 2, 2018. TadA7.10 is also the deaminase domain of ABEmax, which is a variant of ABE7.10 that has been codon optimized for expression in human cells. For instance, the current-generation ABE variant ABE8e (which contains the TadA-8e mutant adenosine deaminase) typically achieves higher editing efficiencies than existing CBEs, despite the strong tRNA substrate preference of wild-type TadA^{9,11,12}. TadA-8e and ABE8e are described in International Publication No. WO 2021/158921, published Aug. 12, 2021.

ABEs have several advantages relative to their CBE counterparts. For instance, compared with most CBE deaminases, TadA enzymes are less processive and therefore typically enable greater single-nucleotide editing precision^{3,7,8,11}. ABEs also offer lower levels of Cas-independent off-target editing compared to CBEs^{8,9,13-15}. This advantage likely arises from tighter unassisted binding of commonly used cytidine deaminases to nucleic acid substrates (the Michaelis constant, Kₘ, for APOBEC1 binding of mRNA is 0.21 nM) compared to that of wild-type TadA (Kₘ=830 nM for a tRNA stem). It also likely arises due to the inability of wild-type TadA to process DNA, and the fact that TadA-8e was evolved using TadA7.10 solely in a Cas-dependent manner. Genome mining¹⁹ and protein engineering have provided alternative cytidine deaminases with lower Cas-independent DNA and RNA editing, but to date, these variants suffer from reduced on-target editing activity and/or larger size^{15,20-24}.

At 166 amino acids in length, evolved TadA adenosine deaminases are substantially smaller than commonly used cytidine deaminases such as APOBEC1 (227 amino acids), AID (182 amino acids)²⁵, CDA (207 amino acids)⁷, or APOBEC3A (198 amino acids)²⁶, making TadA-derived base editors easier to deliver into cells by size-constrained methods and systems, such as AAV. Indeed, the small size of TadA has enabled ABEs, but not CBEs, to be delivered into animal tissues in vivo using a single AAV^{27,28}.

The inventors of the present disclosure hypothesized that directed evolution of an adenosine deaminase to perform cytidine deamination might yield CBEs that maintain high on-target activity but inherit the lower Cas-independent off-target editing and smaller size of current ABEs (e.g., making them easier to deliver into cells by size-constrained methods such as AAV). Accordingly, in some embodiments, the present disclosure provides CBEs that comprise a mutated adenosine deaminase (that preferentially deaminates cytidine in DNA) and a napDNAbp domain (e.g., a Cas9 nickase). The cytidine deaminases evolved from TadA deaminases that are described herein are referred to as “TadA-CDs,” and the CBEs disclosed herein that contain TadA-CDs are referred to herein as “TadCBEs.”

Thus, aspects of the present disclosure relate to a CBE comprising a programmable DNA binding protein (e.g., Cas9) and an evolved deaminase that preferentially deaminates a pyrimidine, and in particular a cytidine, in DNA. For example, the disclosed TadA-CD deaminase variants exhibit ratios of cytidine deamination to adenine deamination of about 10:1, 15:1, 20:1, or more than 20:1. In particular embodiments, the disclosed deaminase variants exhibit ratios of cytidine deamination to adenine deamination of about 20:1. The one or more TadA-CDs deaminases described herein comprise a plurality of mutations, which lie on a loop near the active site, that are critical for switching selectivity for adenosine to cytidine. These mutations impart the TadA-CD with the distinct advantage of the low off-target editing frequencies exhibited by adenosine deaminases used in existing ABEs, such as TadA-8e, while having activity for cytidines in a target region of DNA. They also have the advantage of being size-minimized (e.g., <4.7 kb), which confers the ability to encode TadCBEs containing these deaminase variants in a single AAV vector rather than across two intein-mediated split AAV vectors, or alternatively, using engineered virus-lipid particles (e.g., such as those described herein). In some embodiments, the TadCBEs further comprise any napDNAbp domain useful for cytidine base editing activity, as well as a uracil glycosylase inhibitor (UGI) domain. These TadA-CD variants were generated through continuous and/or non-continuous evolutionary methodologies, including PACE experiments on a TadA-8e substrate (or starting point).

Other aspects of the present disclosure are related to phage-assisted evolution selection systems (e.g., PACE and/or PANCE) to enhance the substrate specificity of adenosine deaminase domains of ABEs for cytosine (where the ABEs contained Cas9 or a Cas9 ortholog). In some embodiments, selection techniques comprise vector systems for PACE evolution that comprise a low-stringency vector and a high-stringency vector. Additional aspects relate to cells containing either of these vectors, or the disclosed vector system. For example, in some embodiments, the highly active adenosine deaminase TadA-8e is evolved (e.g., mutated) to perform cytidine deamination through PACE. The evolved TadA-CDs contain mutations, which lie on a loop near the active site of the deaminase, that are critical for switching selectivity for adenosine to cytidine.

Compared to the most commonly used naturally occurring CBEs, such as BE4max and variants thereof, the disclosed TadCBEs offer comparable or higher on-target activity, smaller size, and/or substantially lower Cas-independent DNA and RNA off-target editing activity, both of which can be further suppressed without decreasing on-target editing by introducing the V106W mutation. These TadCBEs can be used for single or multiplexed base editing at therapeutically relevant genomic loci in mammalian cells, such as primary human T cells and hematopoietic stem and progenitor cells, as demonstrated herein. Other cells are also possible and are disclosed elsewhere herein. The creation of TadCBEs expands the utility of cytosine base editors for gene editing.

In some embodiments, the evolved TadA-CDs may comprise mutations at residues E27, V28, and H96, and may further comprise at least one mutation at a residue selected from R26, M61, Y73, I75, M151, Q154, and A158, in the amino acid sequence of SEQ ID NO: 41 (i.e., TadA-8e deaminase), or corresponding mutations in a homologous adenosine deaminase. Exemplary homologous deaminases include TadA deaminases derived from any of Staphylococcus aureus, Salmonella typhi, Shewanella putrefaciens, Haemophilus influenzae, Caulobacter crescentus, and Bacillus subtilis. As such, in some embodiments, the evolved TadA-CDs may comprise one or more mutations at any of SEQ ID NO: 317-323, 354, and 355 that confer cytidine activity. In some embodiments, the evolved TadA-CDs may comprise one or more mutations at any of SEQ ID NO: 34-40, 42-54, 33, 315, and 326 that confer cytidine activity. The deaminases of the present disclosure may be evolved from any adenosine deaminase reported to date to have adenosine deaminase activity.

In some embodiments, the disclosed TadA-CD variants comprise an amino acid sequence that is at least 80%, 85%, 90%, 95%, 98%, 99%, or 99.5% identical to the amino acid sequence of TadA-8e (SEQ ID NO: 41), wherein the amino acid corresponding to residue 27 of SEQ ID NO: 41 is any amino acid except for E.

In some embodiments, the TadA-CD variants comprise an amino acid sequence that is at least 80%, 85%, 90%, 95%, 98%, 99%, or 99.5% identical to the amino acid sequence of SEQ ID NO: 41, wherein the amino acid corresponding to residue 28 of SEQ ID NO: 41 is any amino acid except for V.

In other embodiments, the TadA-CD variants comprise an amino acid sequence that is at least 80%, 85%, 90%, 95%, 98%, 99%, or 99.5% identical to the amino acid sequence of SEQ ID NO: 41, wherein the amino acid corresponding to residue 96 of SEQ ID NO: 41 is any amino acid except for H.

In some embodiments, the disclosed TadA-CD variants comprise an amino acid sequence that is at least 80%, 85%, 90%, 95%, 98%, 99%, or 99.5% identical to the amino acid sequence of any one of SEQ ID NOs: 34-40. In other embodiments, the TadA-CD variant comprises the amino acid of any one of SEQ ID NOs: 34-40.

The disclosed TadA-CD variants may further comprise a V106W mutation. In some embodiments, the V106W mutation results in adenine base editing of less than or equal to 1.5%, less than or equal to 1%, less than or equal to 0.75%, less than or equal to 0.5%, less than or equal to 0.25%, less than or equal to 0.1%, less than or equal to 0.05%, or less than or equal to 0.01% across targets evaluated (editing frequencies indicated above may represent an average or a maximum).

Other aspects of the present disclosure relate to base editors comprising a programmable DNA binding domain (e.g., napDNAbp) and a disclosed, evolved TadA-CD domain. In some embodiments, the napDNAbp of the base editor is a Cas9 protein, such as a Cas9 nickase. In some embodiments, the napDNAbp of the base editor is an Nme2Cas9 protein (such as an eNme2Cas9 nickase), or Nme2Cas9 variant. In some embodiments, the napDNAbp of the base editor is any of the proteins listed in Table 6. In some embodiments, the base editor further comprises a UGI domain. In some embodiments, the base editor further comprises nuclear localization domains. As such, provided herein are TadCBEs. In another aspect, the present disclosure describes a complex comprising any of the disclosed base editor and a guide RNA bound to the napDNAbp domain of the base editor.

In some aspects, the disclosure relates to TadA-derived cytidine deaminases that provide efficient conversions of target cytosines to thymines and target adenines to guanines (herein referred to as “TadA-dual” deaminases and base editors). TadA-dual deaminases are able to edit C and A bases within a protospacer, and in particular within the editing window of a protospacer. These editors install both A-to-G and C-to-T edits at roughly equivalent efficiencies (e.g., a base editor comprising TadA-dual, SEQ ID NO: 39).

In some embodiments, the TadA-dual deaminase is mutated relative to TadA-8e (SEQ ID NO. 41). In some embodiments, the TadA-dual deaminase comprises a cytidine deaminase comprising one, two, three, four, or five mutations selected from R26G, V28A, A48R, Y73S, and H96N (e.g., TadA-CDf, SEQ ID NO: 39).

In some embodiments, the TadA-dual deaminase is mutated relative to TadA-CDf (SEQ ID NO: 39). In some embodiments, the TadA-dual deaminase comprise a mutation at position N46 of the amino acid sequence of SEQ ID NO: 39. In some embodiments, the Tad-dual deaminase comprises an amino acid sequence that is at least 80%, at least 85%, at least 90%, at least 95%, at least 99%, at least 99.5%, or at least 99.8% identical to the sequence identity of SEQ ID NOs: 39-54.

In some embodiments, the TadA-dual deaminases have an increased affinity for cytosine relative to adenosine. For instance, in some embodiments, the dual editors provide A-to-G and C-to-T editing at a ratio of 0.7:1, 0.8:1, 0.9:1, 1:1, 1.1:1, 1.2:1, 1.3:1, 1.4:1, or 1.5:1. However, in some embodiments, the TadA-dual deaminases have a higher specificity for cytosine than for adenosine.

In other embodiments, the TadA-dual (e.g., SEQ ID NO: 39) deaminases may be further mutated (e.g., using PANCE and/or PACE) to produce cytidine deaminases with an increased affinity for cytosine relative to adenosine. For example, in some embodiments, the ratio of the adenosine deamination activity to the cytidine deamination activity of the deaminase is at least about 0.001:1, 0.005:1, 0.007:1, 0.01:1, 0.05:1, 0.07:1, or 0.1:1.

Additional aspects of the disclosure relate to polynucleotides, vectors, and cells encoding the napDNAbps, cytidine deaminases, and fusion proteins thereof. In some embodiments, the base editors of the current disclosure may be encoded in a polynucleotide as disclosed herein. In some embodiments, the deaminase variants of the current disclosure may be encoded in a polynucleotide as disclosed herein. In certain embodiments, the disclosed vectors comprise a polynucleotide encoding any one of the base editors of the current disclosure. In other embodiments, the disclosure provides cells and compositions that comprise any one of the deaminase variants, base editors, complexes, nucleic acids, or vectors described herein. Also, provided herein are AAV vectors encoding any of the disclosed base editors and optionally a guide RNA.

Other aspects of the disclosure provide pharmaceutical compositions comprising any one of the cytidine deaminases, or variants thereof, base editors, complexes, viruses, nucleic acids, and/or vectors described herein.

In some aspects, the present disclosure encompasses methods comprising contacting a nucleic acid molecule (e.g., DNA) with any one of the base editors or complexes described herein. For example, in some embodiments, the methods comprise contacting any one of the BEs described herein with sgRNA to DNA. The contacting in these methods may be in vivo, in vitro, or ex vivo.

Other embodiments describe methods of using the base editors described herein. In some embodiments, the methods comprise using (a) any of the base editors of the current invention and (b) a guide RNA targeting the base editor of (a) to a target C:G nucleobase pair in a double-stranded DNA molecule in DNA editing. In other embodiments, the methods comprise using the base editors, complexes, or pharmaceutical compositions of the current invention, as a medicament. In certain embodiments, the method comprises using the base editors, complexes, or pharmaceutical compositions of the current invention as a medicament to treat a disease, disorder, or condition, such as sickle cell disease or HIV/AIDS.

In some embodiments, the present disclosure provides methods of selecting (e.g., evolving, engineering, etc.) a cytosine base editor. These methods may comprise evolving an adenosine base editor through several successive rounds of PACE and/or PANCE evolution. In certain embodiments, the method comprises a selection phage encoding a mutated TadA-8e protein fused to a NpuN intein, a first plasmid encoding an NpuC intein fused to dCas9-UGI, a second plasmid encoding a gIII driven by a T7 or proT7 promoter and encoding an sgRNA, and a third plasmid encoding a T7 RNA polymerase-degron fusion.

In another aspect, the present disclosure encompasses methods of generating one or more of the base editors described herein using any of the vectors described herein.

Further aspects of the present disclosure also relate to kits comprising a nucleic acid construct comprising (a) a nucleic acid sequence encoding any one of the base editors described herein, and (b) a nucleic acid sequence encoding a guide RNA. In some embodiments, the nucleic acid construct further comprises one or more heterologous promoters that drive the expression of the sequence of (a) and/or the sequence of (b).

In some aspects, the base editors described herein may be administered to a subject to treat a disease or disorder. Thus, methods are provided wherein the described TadCBEs are administered to a subject, and a target sequence in the genome of the subject is edited. The target sequence may comprise a mutant C:G base pair, e.g., a mutant C:G base pair associated with a disease or disorder. In various embodiments of these methods, the degree of cytidine deamination by the base editor exceeds the degree of adenosine deamination by a factor of 10, 15, 20, or more than 20 (ratios of 10:1, 15:1, 20:1, or more than 20:1).

The disclosure further provides uses of any one of the base editors described herein and a guide RNA targeting this base editor to a target C:G base pair in a nucleic acid molecule in the manufacture of a kit or composition for nucleic acid editing, wherein the nucleic acid editing comprises contacting the nucleic acid molecule with the base editor and guide RNA under conditions suitable for the deamination of the cytosine (C) of the C:G nucleobase pair. The disclosure further provides uses of any one of the base editors described herein and a guide RNA targeting this base editor to a target C:G base pair in a nucleic acid molecule in the manufacture of a kit for evaluating the off-target effects of the base editor.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1E. Phage-assisted evolution of a cytidine deaminase from TadA-8e. (FIG. 1A) Evolutionary trajectory of a TadA-based cytidine deaminase from the tRNA deaminase, TadA. (FIG. 1B) PACE overview. The selection phage (purple) encodes the evolving protein. E. coli hosts (grey) contain 1) a mutagenesis plasmid to diversify the phage (red) and 2) a plasmid system that regulates the expression of pIII (blue, encoded by gIII). Only variants with the desired activity trigger production of pIII and propagate. Phage without the desired activity cannot propagate and are diluted out of the lagoon. (FIG. 1C) Selection circuit for cytidine deamination. TadA-8e variants are encoded on the selection phage (SP, purple). The E. coli harbor four accessory plasmids that establish the selection circuit, in addition to the mutagenesis plasmid: P1 contains the Cas9-UGI components of the base editor. Upon phage infection, the full base editor is reconstituted though the split Npu intein system (yellow). P2 encodes the guide RNA and gIII, which is under transcriptional control of the T7 promoter. P3 contains T7 RNA polymerase that is inactivated by fusion to a degron tag. C·G-to-T·A editing activity inserts a stop codon between T7 RNAP and the degron to yield active T7 RNAP, which leads to transcription of gIII and phage propagation. (FIG. 1D) Two versions of the CBE circuit described herein. In both cases, C·G-to-T·A editing inserts a stop codon before the degron tag, leading to active T7 RNAP. The less stringent circuit requires a C·G-to-T·A edit on the non-coding strand (top) and can tolerate one undesired A to G edit. The more stringent circuit requires a C·G-to-T·A edit on the coding strand and cannot tolerate any undesired A·T-to-G·C edit. (FIG. 1E) Phage-assisted non-continuous evolution of a cytidine deaminase from TadA-8e. The ProD (stronger, less stringent) or ProA (weaker, more stringent) promoter used in each PANCE passage is shown. At each passage, phage are diluted 1:50 unless indicated otherwise. After several rounds of evolution, phage titers stabilize despite increasing dilution rates between passages, suggesting the evolution of cytidine deamination activity.

FIGS. 2A-2D. Evolved TadA* variants catalyze cytidine deamination. (FIG. 2A) Summary of TadA-8e variants evolved and characterized herein. The variants are representative of conserved mutations after nine passages of PANCE or after 159 hours of PACE. For a full list of mutations, see FIGS. 13-14. (FIG. 2B) Method for assessing base editing of target plasmids in E. coli. Cells are co-transformed with a target plasmid (blue) and a base editor plasmid (purple). Base editor expression is induced with arabinose. After 16 hours, cells are harvested, and the target plasmid is analyzed by high-throughput sequencing. (FIG. 2C) Base editing in E. coli of a protospacer matching the selection circuit target site. C·G-to-T·A edits are shown in blue. A·T-to-G·C edits are shown in magenta. Dots represent individual biological replicates and bars represent mean±s.d. from four independent biological replicates. (FIG. 2D) Locations of evolved mutations in the cryo-EM structure of ABE8e (PDB: 6VPC)¹⁸.

FIG. 3. Characterization of evolved TadCBEs with SpCas9 domains in mammalian cells. The specified base editors using SpCas9 nickase domains in the BE4max architecture or ABE8e with 2×UGI were transfected along with each of nine guide RNAs targeting the protospacers shown in each graph. Target cytosines are blue, target adenines are magenta, and PAM sequences are underlined. C·G-to-T·A base editing is shown in shades of blue. A·T-to-G·C base editing is shown in shades of magenta. Dots represent individual values and bars represent mean±s.d. of three independent biological replicates. HEK293T site 3 is abbreviated HEK3, and HEK293T site 4 is abbreviated HEK4.

FIG. 4. Characterization of evolved deaminases with evolved eNme2-C Cas9 domains in mammalian cells. The specified base editors using eNme2-C Cas9 nickase domains (PAM=N₄CN) in the BE4max architecture or ABE8e with 2×UGI were transfected along with each of six guide RNAs targeting the protospacers shown in each graph. Target cytosines are blue, target adenines are magenta, and PAM sequences are underlined. C·G-to-T·A base editing is shown in shades of blue. A·T-to-G·C base editing is shown in shades of magenta. Dots represent individual values and bars represent mean±s.d. of three independent biological replicates.

FIGS. 5A-5D. Characterization of base editing window and Cas-independent off-target DNA and RNA editing by TadCBEs. (FIG. 5A) Base editing activity window for ABE8e with 2×UGI, TadCBEa, and TadCBEa-V106W across nine different target genomic sites in HEK293T. Dots represent average editing across all sites containing the specified base at the indicated position within the protospacer. Individual data points used for this analysis are in FIGS. 2A-2D, FIG. 14, and FIGS. 16A-16B. (FIG. 5B) Method for measuring Cas-independent off-target DNA editing with the orthogonal R-loop assay¹⁵. (FIG. 5C) Average Cas-independent off-target editing across all cytosines within six orthogonal R-loops (SaR1-SaR6) generated by dead S. aureus Cas9. (FIG. 5D) Off-target RNA editing. RNA was harvested from HEK293T cells 48 hours after transfection with the indicated base editor. Following cDNA synthesis, CTNNB1, IP90, and RSL1D1 were amplified and analyzed by high-throughput sequencing. For FIGS. 5C-5D, dots represent individual biological replicates and bars represent mean±s.d. of three independent biological replicates.

FIG. 6. Base editing at therapeutically relevant loci by TadCBEs in primary human T-cells and hematopoietic stem and progenitor cells. mRNA encoding the indicated base editor or GFP as a negative control was electroporated into human T cells (n=4 donors) along with two synthetic guide RNAs targeting (top) CXCR4 or (middle) CCR5 at the specified protospacers. Target cytosines are blue, target adenines are magenta, and PAM sequences are underlined. After 3 days, genomic DNA was harvested from T-cell lysates and analyzed by high-throughput sequencing. The grey boxes indicate the desired location of stop codon installation in CXCR4 and CCR5. The targeted cytidine to yield TAG (CXCR4) and TAA (CCR5) stop codons upon cytosine base editing is underlined. The bottom graph shows that mRNA encoding the indicated base editor or GFP as a negative control was electroporated into hematopoietic stem and progenitor cells along with a synthetic guide RNA targeting the BCL11A enhancer. After 3 days, genomic DNA was harvested from cell lysates and analyzed by high-throughput sequencing. C·G-to-T·A base editing is shown in shades of blue, A·T- to G·C-base editing is shown in shades of magenta. Dots represent individual biological replicates and bars represent mean±s.d. from n=4 donors (top and middle) or n=3 donors (bottom).

FIG. 7. Basis of deamination selectivity selection in PACE and PANCE circuits. In Circuit 1, stop codon formation is only impeded if the base editor deaminates both A₇ and A₈. Circuit 1 is thus tolerant to modest levels of A deamination. In Circuit 2, deamination of a single adenine A₆ will prevent stop codon formation and impede circuit activation and phage propagation. Circuit 2 is thus more stringent for selecting against adenosine deamination.

FIGS. 8A and 8B. PANCE titers and evolved TadA-CD genotypes. (FIG. 8A) Phage titers during PANCE for Lagoons 1-7. Stringency was modulated by increasing the promoter strength from ProD (strongest, least stringent) to ProA (weakest, most stringent), increasing the dilution factor, and by switching from Circuit 1 to Circuit 2. Lagoons 1-6 were inoculated with phage encoding TadA8e-NpuN, while Lagoon 7 was inoculated with phage encoding TadA8e A48R-NpuN. (FIG. 8B) Genotypes from various PANCE lagoons (L1-L7) after PANCE.

FIGS. 9A-9C. PACE titers and evolved TadA-CD genotypes. (FIG. 9A) Phage titers and lagoon flow rate during PACE. Lagoon 1 showed activity-independent propagation in S2060 cells after t=43 hours, signifying phage that evolved selection-independent replication, and was not continued. (FIG. 9B) Genotypes of evolved TadA* variants from lagoon 1 at t=43 hours, before the appearance of selection-independent propagation. (FIG. 9C) Genotypes of evolved TadA* variants from lagoon 2 at various time points.

FIGS. 10A-10C. AlphaFold model of TadA-CDa. (FIG. 10A) The cryo-EM structure of ABE8e (PDB ID 6VPC)¹ is shown bound to DNA containing the 8-azanebularine (8Az) substrate mimic of adenosine. Val 28 (magenta) supports proper positioning of the adenine substrate relative to the catalytic zinc. (FIG. 10B) 8Az was replaced with cytidine using the “Swapna” function in the Chimera software². In the resulting model, C4 of cytosine, which is targeted for nucleophilic attack during deamination, is ˜1 Å away from the target carbon of 8Az, and thus may require shifting of the DNA substrate for productive catalysis. Val 28 may impede this shift of the DNA substrate deeper into the TadA-8e pocket. (FIG. 10C) AlphaFold³ was used to generate a model of evolved TadA-CDa. The ABE8e structure was superimposed to generate a model with the DNA substrate R-loop from 6VPC. The evolved enzyme is not predicted to adopt any apparent differences in secondary structure compared to TadA8e. Evolved replacement of Val 28 in TadA-8e to the smaller Ala or Gly residues found in TadA-CDs may alleviate steric constraints that are predicted to impede productive positioning of the target C4 in cytosine relative to the catalytic zinc ion.

FIG. 11. Indels and C·G-to-G·C editing by SpCas9 variants at nine genomic target sites. The specified base editors using SpCas9 nickase domains in the BE4max architecture or ABE8e with 2×UGI were transfected into HEK293T cells along with each of nine guide RNAs targeting the protospacers shown in each graph. C·G-to-G·C base editing is shown in shades of blue. Indels are shown in grey. Dots represent individual values and bars represent mean±s.d. of three independent biological replicates. The corresponding on-target editing data can be found in FIG. 3.

FIG. 12. Indels and C·G-to-G·C editing by eNme2-C Cas9 variants at six genomic target sites. The specified base editors using eNme2-C Cas9 nickase domains in the BE4max architecture or ABE8e with 2×UGI were transfected into HEK293T cells along with each of six guide RNAs targeting the protospacers shown in each graph. C·G-to-G·C base editing is shown in shades of blue. Indels are shown in grey. Dots represent individual values and bars represent mean±s.d. of three independent biological replicates. The corresponding on-target data are in FIG. 4.

FIG. 13. V106W proximity to TadA-CD mutations. Mutations generated during the evolution of TadA-CDs are shown in blue. Residue V106 is shown in red. The addition of V106W to TadA-7.10, TadA-8e, and TadA-8.17 reduces off-target editing activity⁴⁻⁶. The addition of V106W to TadCBEa-e increases selectivity for deaminating cytidine over adenosine and also reduces off-target editing activity.

FIG. 14. Base editing by V106W variants at six genomic target sites. The specified base editors using SpCas9 nickase domains in the BE4max architecture or ABE8e with 2×UGI were transfected into HEK293T cells along with each of six guide RNAs targeting the protospacers shown in each graph. Target cytosines are blue, target adenines are magenta, and PAM sequences are underlined. C·G-to-T·A base editing is shown in shades of blue. A·T-to-G·C base editing is shown in shades of magenta. Dots represent individual values and bars represent mean±s.d. of three independent biological replicates.

FIG. 15. Indels and C·G-to-G·C editing by V106W variants at six genomic target sites. The specified base editors using SpCas9 nickase domains in the BE4max architecture or ABE8e with 2×UGI were transfected into HEK293T cells along with each of six guide RNAs targeting the protospacers shown in each graph. C·G-to-G·C base editing is shown in shades of blue. Indels are shown in grey. Dots represent individual values and bars represent mean±s.d. of three independent biological replicates.

FIGS. 16A-16B. Base editing, indel formation, and C·G-to-G·C editing by TadA-CD (V106W) variants at three additional genomic target sites. The specified base editors using SpCas9 nickase domains in the BE4max architecture or ABE8e with 2×UGI were transfected into HEK293T cells along with each of three guide RNAs targeting the protospacers shown in each graph. (FIG. 16A) Target cytosines are blue, target adenines are magenta, and PAM sequences are underlined. C·G-to-T·A base editing is shown in shades of blue. A·T-to-G·C base editing is shown in shades of magenta. (FIG. 16B) C·G-to-G·C base editing is shown in shades of blue. Indels are shown in grey. Dots represent individual values and bars represent mean±s.d. of three independent biological replicates.

FIG. 17. Base editing activity windows of CBEs across nine genomic target sites. Dots represent average editing across all sites containing the specified base at the indicated position within the protospacer. Individual data points used for this analysis are in FIGS. 2A-2D, FIG. 14, and FIGS. 16A-16B.

FIG. 18. On-target editing of EMX1 in the Cas-independent R-loop editing experiment. The specified base editors using SpCas9 nickase domains in the BE4max architecture or ABE8e with 2×UGI were transfected into HEK293T cells along with a SpCas9 guide RNA targeting EMX1 as well as the indicated SaCas9 sgRNA. The average on-target C·G-to-T·A base editing across C₅ and C₆ in EMX1 is shown for the indicated base editor. Dots represent individual values and bars represent mean±s.d. of three independent biological replicates. The corresponding Cas-dependent off-target data are shown in FIG. 5C, FIG. 19, and FIG. 20.

FIG. 19. Cas-independent off-target C·G-to-T·A editing at individual sites within six orthogonal R-loops generated by SaCas9. The orthogonal R-loop assay was performed on CBE variants in the BE4max architecture⁷. Cells were transfected with the base editor and one SpCas9 sgRNA targeting the EMX1 locus along with orthogonal dead SaCas9 and one SaCas9 sgRNA corresponding to Sa sites 1-6. Dots represent individual biological replicates and bars represent mean±s.d. from three independent biological replicates. Corresponding on-target data are in FIG. 18.

FIG. 20. Cas-independent off-target C·G-to-T·A editing by TadCBEe V106W at individual sites within six orthogonal R-loops generated by SaCas9. The orthogonal R-loop assay was performed on CBE variants in the BE4max architecture. Cells were transfected with the base editor and one SpCas9 sgRNA targeting the EMX1 locus along with orthogonal dead SaCas9 and one SaCas9 sgRNA corresponding to Sa sites 1-6. Dots represent individual biological replicates and bars represent mean±s.d. from three independent biological replicates.

FIGS. 21A-21C. Cas-independent off-target DNA editing by TadCBEe V106W at six genomic SaCas9 R-loops. The orthogonal R-loop assay was performed on CBE variants in the BE4max architecture. Cells were transfected with the base editor and one SpCas9 sgRNA targeting the EMX1 locus (on-target) along with orthogonal dead SaCas9 and one SaCas9 sgRNA corresponding to Sa sites 1-6 (SaR1-SaR6). FIG. 21A shows on-target editing at the EMX1 locus. FIG. 21B shows the average C·G-to-T·A base editing across all the adenines within the indicated protospacer is depicted on the graph. (FIG. 21C) The average A·T-to-G·C base editing across all the adenines within the indicated protospacer is depicted on the graph. Dots represent individual biological replicates and bars represent mean±s.d. from three independent biological replicates.

FIG. 22. Cas-independent off-target DNA editing at six genomic SaCas9 R-loops. The orthogonal R-loop assay was performed on CBE variants in the BE4max architecture. Cells were transfected with the base editor and one SpCas9 sgRNA targeting the EMX1 locus (on-target) along with orthogonal dead SaCas9 and one SaCas9 sgRNA corresponding to Sa sites 1-6 (SaR1-SaR6). The average A·T-to-G·C base editing across all the adenines within the indicated protospacer is depicted on the graph. Dots represent individual biological replicates and bars represent mean±s.d. from three independent biological replicates.

FIGS. 23A and 23B. Cas-independent off-target RNA editing of all cytosines and adenines examined across three transcripts for TadCBEe V106W. Total RNA was harvested from HEK293T cells 48 hours after transfection with the indicated base editor. Following cDNA synthesis, CTNNB1, IP90, and RSL1D1 were amplified and analyzed by high-throughput sequencing. At the same time, genomic DNA was harvested from the other plate that was transfected in parallel. The genomic DNA was analyzed for on-target editing of EMX1 as a control for base editor activity. FIG. 23A shows on-target editing of EMX1 in samples corresponding to the RNA editing analysis. FIG. 23B shows the average C-to-U (shades of blue) or A-to-I (shades of magenta) Dots represent individual biological replicates and bars represent mean±s.d. of three independent biological replicates.

FIG. 24. On-target editing of EMX1 in the RNA off-target editing experiment. The indicated base editor was transfected into HEK293T cells in two parallel plates. In one plate, RNA was harvested from HEK293T cells 48 hours after transfection with the indicated base editor and analyzed as described in FIGS. 23A-23B. At the same time, genomic DNA was harvested from the other plate that was transfected in parallel. The genomic DNA was analyzed for on-target editing of EMX1 as a control for base editor activity. Dots represent individual biological replicates and bars represent mean±s.d. of three independent biological replicates.

FIG. 25. Cas-dependent editing of known off-target sites for HEK3. The specified base editors using SpCas9 nickase domains in the BE4max architecture or ABE8e with 2×UGI were transfected into HEK293T cells along with a guide RNA targeting HEK293T site 3 (HEK3). 72 hours after transfection, genomic DNA was harvested and known off-target sites were amplified using the primers in Tables 2A-2E. C·G-to-T·A base editing is shown in shades of blue. A·T-to-G·C base editing is shown in shades of magenta. Dots represent individual values and bars represent mean±s.d. of three independent biological replicates.

FIG. 26. Cas-dependent editing of known off-target sites for HEK4. The specified base editors using SpCas9 nickase domains in the BE4max architecture or ABE8e with 2×UGI were transfected into HEK293T cells along with a guide RNA targeting HEK293T site 4 (HEK4). 72 hours after transfection, genomic DNA was harvested and known off-target sites were amplified using the primers in Tables 2A-2E. C·G-to-T·A base editing is shown in shades of blue. A·T-to-G·C base editing is shown in shades of magenta. Dots represent individual values and bars represent mean±s.d. of three independent biological replicates.

FIGS. 27A-27B. Cas-dependent editing of known off-target sites for EMX1. The specified base editors using SpCas9 nickase domains in the BE4max architecture or ABE8e with 2×UGI were transfected into HEK293T cells along with a guide RNA targeting EMX1. 72 hours after transfection, genomic DNA was harvested and known off-target sites were amplified using the primers in Tables 2A-2E. C·G-to-T·A base editing is shown in shades of blue. A·T-to-G·C base editing is shown in shades of magenta. Dots represent individual values and bars represent mean±s.d. of three independent biological replicates. The corresponding on-target data are shown in FIG. 35.

FIG. 28. Cas-dependent editing of known off-target sites for BCL11A. The specified base editors using SpCas9 nickase domains in the BE4max architecture or ABE8e with 2×UGI were transfected into primary human CD34-positive hematopoietic stem and progenitor cells (n=3 donors) along with a guide RNA targeting BCL11A. 72 hours after transfection, genomic DNA was harvested and known off-target sites were amplified using the primers in Tables 2A-2E C·G-to-T·A base editing is shown in shades of blue. A·T-to-G·C base editing is shown in shades of magenta. Dots represent individual values and bars represent mean±s.d. of three independent biological replicates.

FIGS. 29A-29B. C·G-to-G·C editing and indels for T-cell experiments targeting CXCR4 and CCR5. mRNA encoding the indicated base editor or GFP as a negative control was electroporated into primary human T cells (n=4 donors) along with two synthetic guide RNAs targeting (FIG. 29A) CXCR4 or (FIG. 29B) CCR5 at the specified protospacers. After 3 days, genomic DNA was harvested from T-cell lysates and analyzed by high-throughput sequencing. C·G-to-G·C base editing is shown in shades of blue. Indels are shown in grey. Dots represent individual values and bars represent mean±s.d. of three independent biological replicates.

FIG. 30. Cas-dependent off-target editing in T-cell experiments targeting CXCR4 and CCR5. mRNA encoding the indicated base editor or GFP as a negative control was electroporated into primary human T cells (n=4 donors) along with two synthetic guide RNAs targeting CXCR4 or CCR5 at the specified protospacers. After 3 days, genomic DNA was harvested from T-cell lysates and known off-target sites were amplified using the primers in Tables 2A-2E. C·G-to-T·A base editing is shown in shades of blue. A·T-to-G·C base editing is shown in shades of magenta. Dots represent individual values and bars represent mean±s.d. of three independent biological replicates.

FIGS. 31A-31B. C·G-to-G·C editing, indels, and Cas-dependent off-target editing for editing of BCL11A in hematopoietic stem and progenitor cells. mRNA encoding the indicated base editor or GFP as a negative control was electroporated into CD34-positive human hematopoietic stem and progenitor cells (n=3 donors) along a synthetic guide RNA targeting BCL11A at the specified protospacer. After 3 days, genomic DNA was harvested from cell lysates and analyzed by high-throughput sequencing. (FIG. 31A) C·G-to-G·C base editing is shown in shades of blue. Indels are shown in grey. (FIG. 31B) Known Cas-dependent off-target sites were amplified by the primers listed in Tables 2A-2E. C·G-to-G·C base editing is shown in shades of blue. A·T-to-G·C base editing is shown in shades of magenta. Dots represent individual values and bars represent mean±s.d. of three independent biological replicates.

FIGS. 32A-32C. Characterization of TadCBEs using a genomically integrated mESC target sequence library. FIG. 32A shows overall efficiency and selectivity of base editors analyzed through editing of the library. Data show the average fraction of edited sequencing reads across all library members between protospacer positions −9 to 20, where positions 21-23 are the PAM. FIG. 32B shows the editing profiles of BE4max, TadCBEa-d, TadCBEd V106W, and dual base editor TadDE across 10,683 genomically integrated target sites. The editing window is defined as the protospacer positions for which average editing efficiency is ≥30% of the average peak editing efficiency. Window plots for all variants tested in the library experiment can be found in FIG. 39. FIG. 32C shows sequence motifs of TadCBEd and TadCBEd V106W for cytosine and adenine base editing outcomes determined by performing regression on editing efficiencies. Opacity of sequence motifs is proportional to the test R on a held-out set of sequences. Complete sequence motif plots for all variants are shown in FIGS. 41A and 41B.

FIG. 33. Testing individual mutations in TadCBEs. Base editing in E. coli of a protospacer matching the selection circuit target site. Cells are co-transformed with a target plasmid and a base editor plasmid. Base editor expression is induced with arabinose. After 16 hours, cells are harvested, and the target plasmid is analyzed by high-throughput sequencing. (Top graph) Addition of individual mutations identified through evolution to ABE8e is insufficient for generating a CBE. (Middle graph) Analysis of mutations in TadCBEa-c and TadCBEe. Mutations in the loop region of ABE8e imparts selectivity for cytidine deamination, while auxiliary mutations boost activity. (Bottom graph) Analysis of mutations in TadCBEd. C·G-to-T·A edits are shown in blue. A·T-to-G·C edits are shown in magenta. Dots represent individual biological replicates and bars represent mean±s.d. from four independent biological replicates.

FIG. 34. Reversion analysis of TadCBEs. Base editing in E. coli of a protospacer matching the selection circuit target site. Cells are co-transformed with a target plasmid and a base editor plasmid. Base editor expression is induced with arabinose. After 16 hours, cells are harvested, and the target plasmid is analyzed by high-throughput sequencing. (Top graph) Mutations shown are relative to TadCBEa (grey box). (Bottom graph) Mutations shown are relative to TadCBEe (grey box). C·G-to-T·A edits are shown in blue. A·T-to-G·C edits are shown in magenta. Dots represent individual biological replicates and bars represent mean±s.d. from four independent biological replicates.

FIG. 35. On-target editing of EMX1. The specified base editors using SpCas9 nickase domains in the BE4max architecture or ABE8e with 2×UGI were transfected into HEK293T cells along with a guide RNA targeting EMX1. 72 h after transfection, genomic DNA was harvested and known off-target sites were amplified using the primers in Table 1. C·G-to-T·A base editing is shown in shades of blue. A·T-to-G·C base editing is shown in shades of magenta. Dots represent individual values and bars represent mean±s.d. of three independent biological replicates. The corresponding off-target data are shown in FIGS. 27A and 27B.

FIG. 36. On-target and off-target editing of EMX1 by TadCBE V106W. The specified base editors using SpCas9 nickase domains in the BE4max architecture or ABE8e with 2×UGI were transfected into HEK293T cells along with a guide RNA targeting EMX1. 72 h after transfection, genomic DNA was harvested and known off-target sites were amplified using the primers in Table 4. C·G-to-T·A base editing is shown in shades of blue. A·T-to-G·C base editing is shown in shades of magenta. Dots represent individual values and bars represent mean±s.d. of three independent biological replicates.

FIG. 37. Schematic of mESC library experiment Thousands of pairs of sgRNAs and corresponding target sites are integrated into mESCs and treated with base editors. Base editor-containing cells are enriched by antibiotic selection, and library cassettes are amplified for high-throughput sequencing.

FIG. 38. Correlation between replicates in the mESC library experiment. Uncorrected C·G-to-T·A editing efficiency at each target site for each replicate. The red dashed line is a total least-squares regression line.

FIG. 39. Editing windows of TadCBE V106W variants in the mESC library editing experiment. The editing window is defined as positions within the protospacer where the average fraction of converted bases at that position is at least 30% of the average editing at the maximally edited position. C·G-to-T·A base editing is shown in blue. A·T-to-G·C base editing is shown in red.

FIGS. 40A and 40B. Effect of V106W on peak editing in the mESC library experiment. FIG. 40A shows C·G-to-T·A editing efficiency with TadCBEd (with and without the V106W substitution) for each library member containing a cytosine at protospacer position 6. The red dashed line is a total least-squares regression line. FIG. 40B shows A·T-to-G·C editing efficiency with TadCBEd (with and without V106W) for each library member containing an adenine at protospacer position 6. The red dashed line is a total least-squares regression line.

FIGS. 41A and 41B. Sequence motifs for context preferences of TadCBEs. Sequence motifs for base editing activities from performing regression on the editing efficiencies. Logo opacity is proportional to the R on a held-out test set. Plots are provided for C·G-to-T·A base editing (FIG. 41A) and for A·T-to-G·C base editing (FIG. 41B).

FIG. 42. Characterization of evolved deaminases with evolved eNme2-C Cas9 domains. The specified base editors using eNme2-C Cas9 nickase domains (PAM=N₄CN) in the BE4max architecture, or ABE8e with 2×UGI, were transfected into HEK293T cells along with each of six guide RNAs targeting the protospacers shown in each graph. Target cytosines are blue, target adenines are magenta, and PAM sequences are underlined. C·G-to-T·A base editing is shown in shades of blue. A·T-to-G·C base editing is shown in shades of magenta. Dots represent individual values and bars represent mean±s.d. of three independent biological replicates.

FIG. 43. Indels and C·G-to-G·C editing by eNme2-C Cas9 variants at six genomic target sites. The specified base editors using eNme2-C Cas9 nickase domains in the BE4max architecture or ABE8e with 2×UGI were transfected into HEK293T cells along with each of six guide RNAs targeting the protospacers shown in each graph. C·G-to-G·C base editing is shown in shades of blue. Indels are shown in grey. Dots represent individual values and bars represent mean±s.d. of three independent biological replicates. The corresponding on-target data are shown in FIG. 42.

FIG. 44. Characterization of evolved deaminases with SaCas9 domains. The specified base editors using SaCas9 nickase domains (PAM=NNGRRT) in the BE4max architecture or ABE8e with 2×UGI were transfected into HEK293-T cells along with each of nine guide RNAs targeting the protospacers shown in each graph. Target cytosines are blue, target adenines are magenta, and PAM sequences are underlined. C·G-to-T·A base editing is shown in shades of blue. A·T-to-G·C base editing is shown in shades of magenta. Dots represent individual values and bars represent mean±s.d. of three independent biological replicates.

FIG. 45. Indels and C·G-to-G·C editing by SaCas9 variants at nine genomic target sites. The specified base editors using SaCas9 nickase domains in the BE4max architecture or ABE8e with 2×UGI were transfected into HEK293T cells along with each of six guide RNAs targeting the protospacers shown in each graph. C·G-to-G·C base editing is shown in shades of blue. Indels are shown in grey. Dots represent individual values and bars represent mean±s.d. of three independent biological replicates. The corresponding on-target data are shown in FIG. 44.

FIG. 46. Characterization of TadDE with SpCas9 in mammalian cells. The specified base editors using SpCas9 nickase domains (PAM=NGG) in the BE4max architecture or ABE8e with 2×UGI were transfected along with each of nine guide RNAs targeting the protospacers shown in each graph. Target cytosines are blue, target adenines are magenta, and PAM sequences are underlined. C·G-to-T·A base editing is shown in shades of blue. A·T-to-G·C base editing is shown in shades of magenta. Dots represent individual values and bars represent mean±s.d. of three independent biological replicates.

FIG. 47. Indels and C·G-to-G·C editing by SpCas9 variants at nine genomic target sites. The specified base editors using SpCas9 nickase domains in the BE4max architecture or ABE8e with 2×UGI were transfected into HEK293T cells along with each of six guide RNAs targeting the protospacers shown in each graph. C·G-to-G·C base editing is shown in shades of blue. Indels are shown in grey. Dots represent individual values and bars represent mean±s.d. of three independent biological replicates. The corresponding on-target data are in FIG. 46.

FIG. 48. On-target editing of V106W variants for T-cell experiments targeting CXCR4 and CCR5. mRNA encoding the indicated base editor or GFP as a negative control was electroporated into human T cells (n=4 donors) along with two synthetic guide RNAs targeting (top) CXCR4 or (bottom) CCR5 at the specified protospacers. Target cytosines are blue, target adenines are magenta, and PAM sequences are underlined. After 3 days, genomic DNA was harvested from T-cell lysates and analyzed by high-throughput sequencing. The grey boxes indicate the desired location of stop codon installation in CXCR4 and CCR5. The targeted cytidine to yield TAG (CXCR4) and TAA (CCR5) stop codons upon cytosine base editing is underlined. Dots represent individual values and bars represent mean±s.d. of three independent biological replicates.

FIG. 49. C·G-to-G·C editing and indels for T-cell experiments targeting CXCR4 and CCR5 with TadCBEe V106W variants. mRNA encoding the indicated base editor or GFP as a negative control was electroporated into primary human T cells (n=4 donors) along with two synthetic guide RNAs targeting (top) CXCR4 or (bottom) CCR5 at the specified protospacers. After 3 days, genomic DNA was harvested from T-cell lysates and analyzed by high-throughput sequencing. C·G-to-G·C base editing is shown in shades of blue. Indels are shown in grey. Dots represent individual values and bars represent mean±s.d. of three independent biological replicates.

FIG. 50. Cas-dependent off-target editing in T-cell experiments targeting CXCR4 and CCR5 with TadCBEe V106W variants. mRNA encoding the indicated base editor or GFP as a negative control was electroporated into primary human T cells (n=4 donors) along with two synthetic guide RNAs targeting (top) CXCR4 or (bottom) CCR5 at the specified protospacers. After 3 days, genomic DNA was harvested from T-cell lysates and known off-target sites were amplified using the primers in Table 4. C·G-to-T·A base editing is shown in shades of blue. A·T-to-G·C base editing is shown in shades of magenta. Dots represent individual values and bars represent mean±s.d. of three independent biological replicates.

FIGS. 51A-51E. Prophetic use of an active and selective cytosine base editor for stop codon installation at disease-relevant sites. Residual A-to-G editing prevents correct stop codon installation (FIG. 51A). Schematic of the evolution of a cytosine base editor from a TadA dual base editor (TadA-DE) (FIG. 51B). Diagram depicting phage-assisted continuous evolution, or PACE (left) and the selection circuit, as used according to some embodiments (right). In some embodiments, a continuous flow of E. coli host cells with the selection circuit and a mutagenesis plasmid (red) are infected by selection phage encoding a partial deaminase (SP). In this particular embodiment of the selection circuit, phage propagation is linked with the expression of gIII (P2), which can only be transcribed with active T7 RNA polymerase. In some embodiments, a T7 RNA polymerase (P3) is fused to a C-terminal degron, and the deaminase must perform C-to-U editing to install a stop codon before the degron, yielding active T7 RNA polymerase. In the event of phage infection, the full deaminase is completed using a split-intein system (P1) and mutations can occur on the deaminase. Beneficial mutations lead to phage propagation and enrichment in the lagoon, while the less-fit phage are unable to propagate and are subsequently washed out by the constant outflow (FIG. 51C). Evolution trajectory of an active and selective cytosine base editor from TadA-DE. Phage-assisted non-continuous evolution (PANCE) was performed on TadA-DE until phage titers increased despite higher stringency from dilution factor and promoter strength, indicating that beneficial mutations have occurred. The resulting variant identified a conserved mutation at position N46 in the deaminase, so an NNK library was constructed at position N46, and PANCE was performed on these variants. To increase stringency even further, PACE was performed for >100 hrs on the resulting variants from both PANCEs. Dilution factors are indicated on the right y-axis (FIG. 51D). Cryo-EM structure of ABE8e (PDB: 6VPC) with new conserved mutations labeled (FIG. 51E).

FIGS. 52A-52E. Genotypes from PANCE lagoons (L1-L2) after PANCE (FIG. 52B). Genotypes from PANCE lagoons (L1-L3) after PANCE using an NNK library at N46 (FIG. 52B). Genotypes from PACE lagoon (L1) after PACE using an NNK library at N46 (FIG. 52C). Genotypes at various timepoints from PACE lagoon (L1) after PACE using an NNK library at N46 (FIG. 52D). Genotypes at various timepoints from PACE lagoon (L2) after PACE using an NNK library at N46 (FIG. 52E). Select sequences shown in FIG. 52A.

FIG. 53. Profiling the activity and sequence context specificity of TadCBEs in E. coli. The bars indicate the average activity of CBE variants when tested on a library of substrates designed to contain the target base (A or C) at protospacer positions 6 with the 5′ and 3′ base varied as A, T, C, or G. Each dot represents the percentage of sequencing reads containing the specified edit for a given sequence context. The dots are colored according to the 5′ context of the base (A, red; C, green; G, blue; T, yellow). The mutations in the newly evolved mutations are listed relative to TadDE. TadDE=TadA8e R26G V28A A48 Y73S H96N. While eTdCBEmax, CBET-1.52, TadCBEd, TadDE N46I Y73P, and TadDE N46C Y73P, displayed lower activity depending on the sequence context, the evolved variants TadDE N46V Y73P and TadDE N46L Y73P display over 80% editing regardless of sequence context.

FIGS. 54A and 54B. Comparison of the evolved active and selective cytosine base editors with existing cytosine base editors in mammalian cells. TadDE N46 variants along with existing cytosine base editors with SpCas9 nickases in the BE4max architecture were transfected into HEK293T cells with guide RNAs targeting three protospacers. TadDE N46 variants show comparable on-target activity with no residual A-to-G editing. Dots represent individual values from independent biological replicates. PAM sequences are underlined. HEK293T Site 2 is abbreviate HEK2, and HEK293T Site 4 is abbreviated HEK4 (FIG. 54A). TadDE N46 variants along with existing cytosine base editors with eNme-Cas9 nickases in the BE4max architecture were transfected into HEK293T cells with guide RNAs targeting two protospacers. TadDE N46 variants show higher or comparable on-target activity with no residual A-to-G editing. Dots represent individual values from independent biological replicates. PAM sequences are underlined (FIG. 54B).

FIGS. 55A and 55B. Cas9-independent and RNA off-target editing by TadCBEs. Average Cas9-independent off-target editing across all cytosines for four orthogonal R-loops (SaR1-SaR4) generated by a dead S. aureus Cas9. The mutations in the newly evolved mutations are listed relative to TadDE. TadDE N46 variants show similar off-target editing compared to TadCBEd. Dots represent individual values from independent biological replicates (FIG. 55A). Off-target RNA editing. TadDE N46 variants show similar off-target editing compared to TadCBEd. Dots represent individual values from independent biological replicates (FIG. 55B).

FIG. 56. Stop codon installation at therapeutically-relevant loci by TadCBEs in HEK293 Ts. TadCBEs were used to install stop codons in PCSK9, which is a therapeutic strategy that is being explored for lowering blood cholesterol. The gray boxes indicate the desired location of stop codon installation. The mutations in the newly evolved mutations are listed relative to TadDE. Residual A-to-G editing from TadCBEd causes stop codon erasure, demonstrating that the lack of residual A-to-G in the TadDE N46 variants is critical for stop codon installation. Dots represent individual values from independent biological replicates. PAM sequences are underlined.

FIG. 57. On-target and Cas-dependent editing of known off-target sites for HEK3. TadDE N46 variants along with existing cytosine base editors with SpCas9 nickases in the BE4max architecture were transfected into HEK293T cells with a guide RNA targeting HEK3. The mutations in the newly evolved mutations are listed relative to TadDE. TadDE N46 variants show similar off-target editing compared to TadCBEd. Dots represent individual values from independent biological replicates.

FIG. 58. On-target and Cas-dependent editing of known off-target sites for HEK4. TadDE N46 variants along with existing cytosine base editors with SpCas9 nickases in the BE4max architecture were transfected into HEK293T cells with a guide RNA targeting HEK4. The mutations in the newly evolved mutations are listed relative to TadDE. TadDE N46 variants show similar off-target editing compared to TadCBEd. Dots represent individual values from independent biological replicates.

FIG. 59. On-target and Cas-dependent editing of known off-target sites for EMX1. TadDE N46 variants along with existing cytosine base editors with SpCas9 nickases in the BE4max architecture were transfected into HEK293T cells with a guide RNA targeting EMX1. The mutations in the newly evolved mutations are listed relative to TadDE. TadDE N46 variants show similar off-target editing compared to TadCBEd. Dots represent individual values from independent biological replicates.

FIG. 60. On-target and Cas-dependent editing of known off-target sites for BCL11a. TadDE N46 variants along with existing cytosine base editors with SpCas9 nickases in the BE4max architecture were transfected into HEK293T cells with a guide RNA targeting BCL11a. The mutations in the newly evolved mutations are listed relative to TadDE. TadDE N46 variants show similar off-target editing compared to TadCBEd. Dots represent individual values from independent biological replicates.

FIG. 61. On-target editing at EMX1 correlated to Cas-independent off-target editing. TadDE N46 variants along with existing cytosine base editors with SpCas9 nickases in the BE4max architecture were transfected into HEK293T cells with an SpCas9 guide RNA targeting EMX1 along with an SaCas9 guide RNA. The mutations in the newly evolved mutations are listed relative to TadDE. Dots represent individual values from independent biological replicates.

FIG. 62. On-target editing at EMX1 correlated to RNA off-target editing. TadDE N46 variants along with existing cytosine base editors with SpCas9 nickases in the BE4max architecture were transfected into HEK293T cells in two plates. In one plate, RNA was harvested 48 hours after transfection, and in the other plate, genomic DNA was harvested. The genomic DNA was analyzed for on-target editing of EMX1. The mutations in the newly evolved mutations are listed relative to TadDE. TadDE N46 variants show similar off-target editing compared to TadCBEd. Dots represent individual values from independent biological replicates.

FIG. 63. Continuation of FIG. 53. Each graph in FIG. 53 is also represented in FIG. 63; however, each data point in FIG. 53 (represented as a dot) is shown as a bar in FIG. 63.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

### AAV

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

rAAV particles may comprise a nucleic acid vector (e.g., a recombinant genome), which may comprise at a minimum: (a) one or more heterologous nucleic acid regions comprising a sequence encoding a protein or polypeptide of interest (e.g., a split Cas9 or split nucleobase) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

### Deaminases

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deaminase is a cytidine (or cytosine) deaminase, which catalyzes the hydrolytic deamination of cytidine or cytosine.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

As used herein, the term “adenosine deaminase” or “adenosine deaminase domain” refers to a protein or enzyme that catalyzes a deamination reaction of an adenosine (or adenine). The terms “adenosine” and “adenine” are used interchangeably for purposes of the present disclosure. For example, for purposes of the disclosure, reference to an “adenine base editor” (ABE) refers to the same entity as an “adenosine base editor” (ABE). Similarly, for purposes of the disclosure, reference to an “adenine deaminase” refers to the same entity as an “adenosine deaminase.” However, the person having ordinary skill in the art will appreciate that “adenine” refers to the purine base whereas “adenosine” refers to the larger nucleoside molecule that includes the purine base (adenine) and sugar moiety (e.g., either ribose or deoxyribose). In certain embodiments, the disclosure provides base editor fusion proteins comprising one or more adenosine deaminase domains. For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminase can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

As used herein, the term “cytidine deaminase” or “cytidine deaminase domain” refers to a protein or enzyme that catalyzes a deamination reaction of a cytidine or cytosine. The terms “cytidine” and “cytosine” are used interchangeably for purposes of the present disclosure. For example, for purposes of the disclosure, reference to an “cytosine base editor” (CBE) refers to the same entity as an “cytosine base editor” (CBE). Similarly, for purposes of the disclosure, reference to an “cytidine deaminase” refers to the same entity as an “cytosine deaminase.” However, the person having ordinary skill in the art will appreciate that “cytosine” refers to the pyrimidine base whereas “cytidine” refers to the larger nucleoside molecule that includes the pyrimidine base (cytosine) and sugar moiety (e.g., either ribose or deoxyribose). A cytidine deaminase is encoded by the CDA gene and is an enzyme that catalyzes the removal of an amine group from cytidine (i.e., the base cytosine when attached to a ribose ring, i.e., the nucleoside referred to as cytidine) to uridine (C to U) and cytidine to deoxyuridine (C to U). A non-limiting example of a cytidine deaminase is APOBEC1 (“apolipoprotein B mRNA editing enzyme, catalytic polypeptide 1”). Another example is AID (“activation-induced cytidine deaminase”). Under standard Watson-Crick hydrogen bond pairing, a cytosine base hydrogen bonds to a guanine base. When cytidine is converted to uridine (or cytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of a C·G pairing to a T·A pairing in the double-stranded DNA molecule.

### Antisense Strand

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Base Editing

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

### Base Editor

The term “base editor (BE)” as used herein, refers to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, T to G). In some embodiments, the base editor is capable of deaminating a base within a nucleic acid such as a base within a DNA molecule. In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017, and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)).

As used herein the terms cytidine, cytosine, and deoxycytidine all synonymous and refer to a cytidine that is able to be edited using a CBE. Likewise, the terms adenosine, adenine, and deoxyadenine all refer to an adenine that is able to be edited using an ABE. Further, the terms cytidine base editor, cytosine base editor, and the like are synonymous. Similarly, the terms adenosine base editor, adenine base editor, and the like are synonymous.

In some embodiments, a nucleobase editor is a macromolecule or macromolecular complex that results primarily (e.g., more than 80%, more than 85%, more than 90%, more than 95%, more than 99%, more than 99.9%, or 100%) in the conversion of a nucleobase in a polynucleic acid sequence into another nucleobase (i.e., a transition or transversion) using a combination of 1) a nucleotide-, nucleoside-, or nucleobase-modifying enzyme; and 2) a nucleic acid binding protein that can be programmed to bind to a specific nucleic acid sequence.

In some embodiments, the nucleobase editor comprises a DNA binding domain (e.g., a programmable DNA binding domain such as a dCas9 or nCas9) that directs it to a target sequence. In some embodiments, the nucleobase editor comprises a nucleobase modifying enzyme fused to a programmable DNA binding domain (e.g., a dCas9 or nCas9). A “nucleobase modifying enzyme” is an enzyme that can modify a nucleobase and convert one nucleobase to another (e.g., a deaminase such as a cytidine deaminase or an adenosine deaminase). In some embodiments, the nucleobase editor may target cytosine (C) bases in a nucleic acid sequence and convert the C to thymine (T) base. In some embodiments, the C to T editing is carried out by a deaminase, e.g., a cytidine deaminase. Base editors that can carry out other types of base conversions (e.g., adenosine (A) to guanine (G), C to G) are also contemplated.

Nucleobase editors that convert a C to T, in some embodiments, comprise a cytidine deaminase. A “cytidine deaminase” refers to an enzyme that catalyzes the chemical reaction “cytosine+H₂O→uracil+NH₃” or “5-methyl-cytosine+H2O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T nucleobase editor comprises a dCas9 or nCas9 fused to a cytidine deaminase. In some embodiments, the cytidine deaminase domain is fused to the N-terminus of the dCas9 or nCas9. In some embodiments, the nucleobase editor further comprises a domain that inhibits uracil glycosylase, and/or a nuclear localization signal. Such nucleobase editors have been described in the art, e.g., in Rees & Liu, Nat Rev Genet. 2018; 19(12):770-788 and Koblan et al., Nat Biotechnol. 2018; 36(9):843-846; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018, International Application No PCT/US2019/033848, filed May 23, 2019, International Application No. PCT/US2019/47996, filed Aug. 23, 2019; International Application No. PCT/US2019/049793, filed Sep. 5, 2019; U.S. Provisional Application No. 62/835,490, filed Apr. 17, 2019; International Application No. PCT/US2019/61685, filed Nov. 15, 2019; International Application No. PCT/US2019/57956, filed Oct. 24, 2019; U.S. Provisional Application No. 62/858,958, filed Jun. 7, 2019; International Publication No. PCT/US2019/58678, filed Oct. 29, 2019, the contents of each of which are incorporated herein by reference in their entireties.

In some embodiments, a nucleobase editor converts an A to G. In some embodiments, the nucleobase editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known adenosine deaminases that act on DNA. Instead, known adenosine deaminase enzymes only act on RNA (tRNA or mRNA). Evolved adenosine deaminase enzymes that accept DNA substrates and deaminate dA to deoxyinosine have been described, e.g., in PCT Application PCT/US2017/045381, filed Aug. 3, 2017, which published as WO 2018/027078, and PCT Application No. PCT/US2019/033848, which published as WO 2019/226953, each of which is herein incorporated by reference by reference.

Exemplary adenine base editors (ABEs) (or “adenosine base editors”) and cytosine base editors (CBEs) (or “cytosine base editors”) are also described in Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 200). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 200). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 200). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 200).

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

cDNA

The term “cDNA” refers to a strand of DNA copied from an RNA template. cDNA is complementary to the RNA template.

### Circular Permutant

As used herein, the term “circular permutant” refers to a protein or polypeptide (e.g., a Cas9) comprising a circular permutation, which is change in the protein's structural configuration involving a change in order of amino acids appearing in the protein's amino acid sequence. In other words, circular permutants are proteins that have altered N- and C-termini as compared to a wild-type counterpart, e.g., the wild-type C-terminal half of a protein becomes the new N-terminal half. Circular permutation (or CP) is essentially the topological rearrangement of a protein's primary sequence, connecting its N- and C-terminus, often with a peptide linker, while concurrently splitting its sequence at a different position to create new, adjacent N- and C-termini. The result is a protein structure with different connectivity, but which often can have the same overall similar three-dimensional (3D) shape, and possibly include improved or altered characteristics, including, reduced proteolytic susceptibility, improved catalytic activity, altered substrate or ligand binding, and/or improved thermostability. Circular permutant proteins can occur in nature (e.g., concanavalin A and lectin). In addition, circular permutation can occur as a result of posttranslational modifications or may be engineered using recombinant techniques (e.g., see, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference).

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered to incorporate embodiments of both the crRNA and tracrRNA into a single RNA species—the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### Degron

The term “degron” or “degron domain” refers to a portion of a polypeptide that influence, controls, directs, or otherwise regulates the rate of degradation of the polypeptide. Degrons can be highly variable and can include short amino acid sequences, structural motifs, and/or exposed amino acids. Also, degrons may be positioned at any location within a polypeptide (e.g., at the N-terminus, the C-terminus, or at an internal position within the primary structure). The particular mechanism of degradation of a polypeptide which is regulated by the degron is not limited and can include ubiquitin-dependent degradation (i.e., degradation that involves proteasomal-based degradation) or ubiquitin-independent degradation. For example, the 4-amino acid sequence tail of NH3-EMLA-COOH (SEQ ID NO: 384) encoded by exon 8 of the SMN2 gene functions as a degron, triggering degradation of SMN2.

### Effective Amount

The term “effective amount,” as used herein, refers to an amount of a biologically active agent that is sufficient to elicit a desired biological response. For example, in some embodiments, an effective amount of a base editor may refer to the amount of the base editor that is sufficient to edit a target site nucleotide sequence, e.g., a genome. In some embodiments, an effective amount of a base editor provided herein, e.g., of a base editor comprising a Cas9 nickase domain and a nucleobase modification domain (e.g., a deaminase domain) may refer to the amount of the base editor that is sufficient to induce editing of a target site specifically bound and edited by the base editor. In some embodiments, an effective amount of a base editor provided herein may refer to the amount of the base editor sufficient to induce editing having the following characteristics: >50% product purity, <5% indels over regions immediately surrounding the target sequence, and/or an editing window of 2-8 nucleotides. In other embodiments, an effective amount of a base editor may refer to the amount of the base editor sufficient to induce editing of >45% product purity, <10% indels, a ratio of intended point mutations to indels that is at least 5:1, and/or an editing window of 2-10 nucleotides. As will be appreciated by the skilled artisan, the effective amount of an agent, e.g., a base editor, a nuclease, a deaminase, a hybrid protein, a complex of a protein and a polynucleotide, or a polynucleotide (e.g., gRNA), may vary depending on various factors, such as, for example, on the desired biological response, e.g., on the specific allele, genome, or target site to be edited, on the target cell or tissue (i.e., the cell or tissue to be edited), and on the agent being used.

### Off-Target Editing and On-Target Editing

The term “off-target editing,” as used herein, refers to the introduction of unintended modifications (e.g., deaminations) to nucleotides (e.g. cytosine) in a sequence outside the canonical base editor binding window (i.e., from one protospacer position to another, typically 2 to 8 nucleotides long). Off-target editing can result from weak or non-specific binding of the gRNA sequence to the target sequence. Off-target editing can also result from intrinsic association of the nucleotide modification domain (e.g. deaminase domain) of a base editor to nucleobases in loci unrelated to the target sequence.

The term “Cas9-dependent off-target editing” refers to the introduction of unintended modifications that result from weak or non-specific binding of a Cas9-gRNA complex (e.g., a complex between a gRNA and the base editor's Cas9 domain) to nucleic acid sites that have fairly high (e.g. more than 60%, or having fewer than 6 mismatches relative to) sequence identity to a target sequence. In contrast, the term “Cas9-independent off-target editing” refers to the introduction of unintended modifications that result from weak associations of a base editor (e.g., the nucleotide modification domain) to nucleic acid sites that do not have high sequence identity (about 60% or less, or having 6-8 or more mismatches relative to) to a target sequence. Because these associations occur independent of any hybridization between the Cas9-gRNA complex and the relevant nucleic acid site, they are referred to as “Cas9-independent.”

The term “on-target editing,” as used herein, refers to the introduction of intended modifications (e.g., deaminations) to nucleotides (e.g., cytosine) in a target sequence, such as using the base editors described herein.

The terms “on-target editing frequency” and “on-target editing efficiency”, as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g., deamination) of a specific nucleotide within DNA, without generating a large number or percentage of insertions or deletions (i.e., indels). It is generally accepted that editing while generating less than 5% indels over regions immediately surrounding the target sequence (as measured over total target nucleotide substrates) constitutes high editing efficiency. The generation of more than 20% indels is generally accepted as poor or low editing efficiency.

The term “off-target editing frequency,” as used herein, refers to the number or proportion of unintended base pairs that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and/or whole genome sequencing (WGS). Off-target effects of the disclosed base editors may be measured using assays and methods disclosed in and International Application No. PCT/US2020/624628, filed Nov. 25, 2020, incorporated herein by reference.

### Functional Equivalent

The term “functional equivalent” refers to a second biomolecule that is equivalent in function, but not necessarily equivalent in structure to a first biomolecule. For example, a “Cas9 equivalent” refers to a protein that has the same or substantially the same functions as Cas9, but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally-occurring, engineered, circular permutant, mutated, or synthetic version of protein X which bears an equivalent function.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof fused to an adenosine deaminase. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### Guide Nucleic Acid

The term “guide nucleic acid” or “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system.

Guide RNA is a particular type of guide nucleic acid which is mostly commonly associated with a Cas protein of a CRISPR-Cas9 and which associates with Cas9, directing the Cas9 protein to a specific sequence in a DNA molecule that includes complementarity to protospace sequence of the guide RNA. As used herein, a “guide RNA” refers to a synthetic fusion of the endogenous bacterial crRNA and tracrRNA that provides both targeting specificity and scaffolding and/or binding ability for Cas9 nuclease to a target DNA. This synthetic fusion does not exist in nature and is also commonly referred to as an sgRNA. However, this term also embraces the equivalent guide nucleic acid molecules that associate with Cas9 equivalents, homologs, orthologs, or paralogs, whether naturally-occurring or non-naturally-occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospacer sequence of the guide RNA. However, this term also embraces the equivalent guide nucleic acid molecules that associate with Cas9 equivalents, homologs, orthologs, or paralogs, whether naturally-occurring or non-naturally-occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein.

Guide RNAs may comprise various structural elements that include, but are not limited to (a) a spacer sequence—the sequence in the guide RNA (having ˜20 nts in length) which binds to a complementary strand of the target DNA (and has the same sequence as the protospacer of the DNA) and (b) a gRNA core (or gRNA scaffold or backbone sequence)—refers to the sequence within the gRNA that is responsible for Cas9 binding, it does not include the ˜20 bp spacer sequence that is used to guide Cas9 to target DNA.

As used herein, the “guide RNA target sequence” refers to the ˜20 nucleotides that are complementary to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA and the protospacer is DNA).

As used herein, the “guide RNA scaffold sequence” refers to the sequence within the gRNA that is responsible for Cas9 binding, it does not include the 20 bp spacer/targeting sequence that is used to guide Cas9 to target DNA.

### Host Cell

The term “host cell,” as used herein, refers to a cell that can host, replicate, and transfer a phage vector useful for a continuous evolution process as provided herein. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

### Inteins and Split-Inteins

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.”

Split inteins are a sub-category of inteins. Unlike the more common contiguous inteins, split inteins are transcribed and translated as two separate polypeptides, the N-intein and C-intein, each fused to one extein. Upon translation, the intein fragments spontaneously and non-covalently assemble into the canonical intein structure to carry out protein splicing in trans.

Inteins and split inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. O., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

### Linker

The term “linker,” as used herein, refers to a chemical group or a molecule linking two molecules or domains, e.g. dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker. In some embodiments, the linker is a 32-amino acid linker. In other embodiments, the linker is a 30-, 31-, 33- or 34-amino acid linker.

### Mutation

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

napDNAbp

The term “napDNAbp,” which stands for “nucleic acid programmable DNA binding protein” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally-occurring or non-naturally-occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

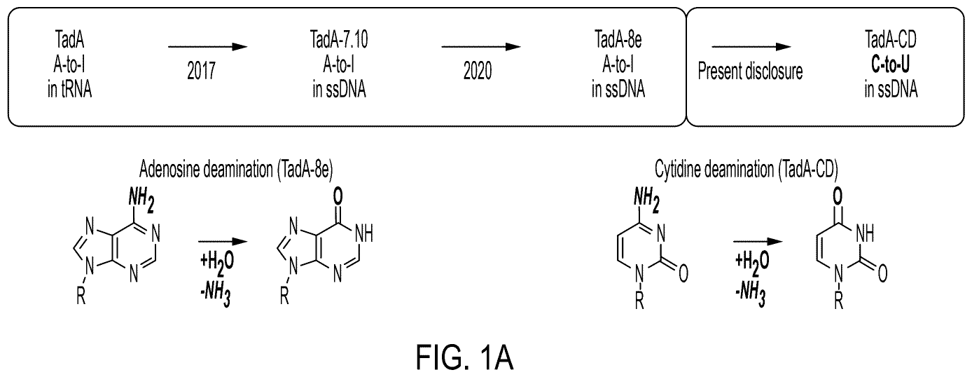

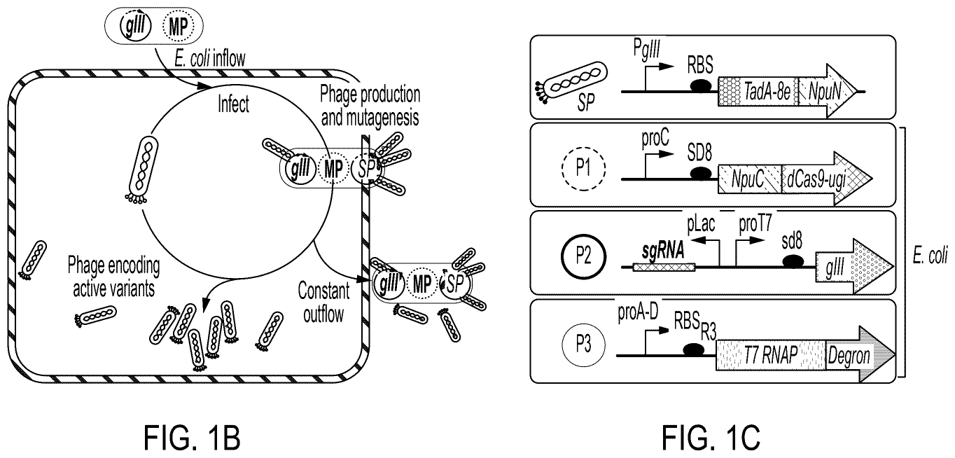

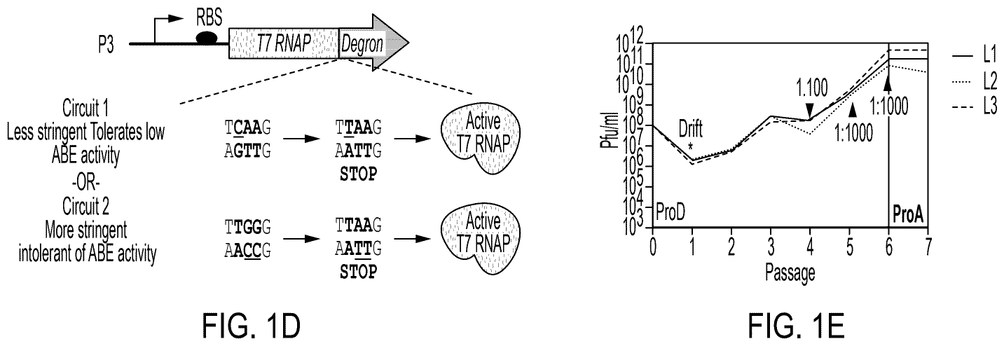

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

### Nickase

The term “nickase” refers to a napDNAbp having only a single nuclease activity (e.g., one of the two nuclease domain is inactivated) that cuts only one strand of a target DNA, rather than both strands. Thus, a nickase type napDNAbp does not leave a double-strand break. In some embodiments, any of the disclosed base editors or vectors may comprise an S. pyogenes Cas9 nickase (SpCas9n, or nCas9) containing a D10A mutation. In some embodiments, any of the disclosed base editors may comprise an Nme2Cas9 nickase (Nme2Cas9n) containing a D16A mutation.

### Nuclear Localization Signal

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example more than 25, 25, 15, 12, 10, 8, 7, 6, 5, or 4 amino acids, but will preferably comprise at least a four to eight amino acid sequence known to function as a nuclear localization signal (NLS).

### Nucleic Acid Molecule

The term “nucleic acid molecule” as used herein, refers to RNA as well as single and/or double-stranded DNA. Nucleic acid molecules may be naturally-occurring, for example, in the context of a genome, a transcript, an mRNA, tRNA, rRNA, siRNA, snRNA, a plasmid, cosmid, chromosome, chromatid, or other naturally-occurring nucleic acid molecule. On the other hand, a nucleic acid molecule may be a non-naturally-occurring molecule, e.g. a recombinant DNA or RNA, an artificial chromosome, an engineered genome, or fragment thereof, or a synthetic DNA, RNA, DNA/RNA hybrid, or including non-naturally-occurring nucleotides or nucleosides. Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, adenosine, deoxythymidine, deoxyguanosine, and cytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, O(6)-methylguanine, and 2-thiocytidine); chemically modified bases; biologically modified bases (e.g. methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

### PACE

The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference.

### Promoter

The term “promoter” is art-recognized and refers to a nucleic acid molecule with a sequence recognized by the cellular transcription machinery and able to initiate transcription of a downstream gene. A promoter may be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters is inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the disclosure provides vectors with appropriate promoters for driving expression of the nucleic acid sequences encoding the fusion proteins (or one or more individual components thereof).

### Product Purity

The term “product purity,” as used herein, refers to the percentage of desired products over total products of a base editing reaction. For instance, product purity of a CBE may be measured as the percentage of total edited sequencing reads (reads in which a target C has been converted to a different base) in which the target C is edited to a T, over a portion of interest of the nucleic acid. Product purity embraces the absence of indels, as well as the desired product of a base conversion.

The term “R-loop” refers to a triplex structure wherein the two strands of a double-stranded DNA are separated for a stretch of nucleotides and held apart by a single-stranded RNA molecule (e.g., gRNA). R-loop formation may be induced by the hybridization of a gRNA having complementarity to the DNA, in association with a napDNAbp protein or domain (e.g., Cas9). Two R-loops are referred to as “orthogonal” when the mechanisms (e.g., napDNAbp-gRNA complexes) that generate their formation function independently of one another.

### Protospacer

As used herein, the term “protospacer” refers to the sequence (˜20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 200, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

### Sense Strand

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Spacer Sequence

As used herein, the term “spacer sequence” in connection with a guide RNA refers to the portion of the guide RNA of about 20 nucleotides which contains a nucleotide sequence that is complementary to the protospacer sequence in the target DNA sequence. The spacer sequence anneals to the protospacer sequence to form a ssRNA/ssDNA hybrid structure at the target site and a corresponding R loop ssDNA structure of the endogenous DNA strand that is complementary to the protospacer sequence.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is a plant.

### Target Site

The term “target site” refers to a sequence within a nucleic acid molecule that is edited by a fusion protein (e.g. a dCas9-deaminase fusion protein provided herein). The target site further refers to the sequence within a nucleic acid molecule to which a complex of the fusion protein and gRNA binds.

### Transcriptional Terminator

A “transcriptional terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In prokaryotic systems, terminators usually fall into two categories (1) rho-independent terminators and (2) rho-dependent terminators. Rho-independent terminators are generally composed of palindromic sequence that forms a stem loop rich in G-C base pairs followed by several T bases. Without wishing to be bound by theory, the conventional model of transcriptional termination is that the stem loop causes RNA polymerase to pause, and transcription of the poly-A tail causes the RNA:DNA duplex to unwind and dissociate from RNA polymerase.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator, spy, yejM, secG-leuU, thrLABC, rrnB T1, hisLGDCBHAFI, metZWV, rrnC, xapR, aspA and arcA terminator. In some embodiments, the termination signal may be a sequence that cannot be transcribed or translated, such as those resulting from a sequence truncation.

### Transition

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transversions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Uracil Glycosylase Inhibitor

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 272. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 272. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 272. In some embodiments, a UGI comprises an amino acid sequence homologous to the amino acid sequence set forth in SEQ ID NO: 272, or an amino acid sequence homologous to a fragment of the amino acid sequence set forth in SEQ ID NO: 272. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 272. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 272. In some embodiments, the UGI comprises the following amino acid sequence: MTNLSDIIEKETGKQLVIQESILMLPEEVEEVIGNKPESDILVHTAYDESTDENVMLL TSDAPEYKPWALVIQDSNGENKIKML (SEQ ID NO: 272) (P14739|UNGI_BPPB2 Uracil-DNA glycosylase inhibitor).

### Variant

As used herein, the term “variant” refers to a protein having characteristics that deviate from what occurs in nature that retains at least one functional i.e. binding, interaction, or enzymatic ability, and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. The term also encompasses circular permutants, mutants, truncations, or domains of a reference sequence, and which display the same or substantially the same functional activity or activities as the reference sequence. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g. SMN protein).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a SMN protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene or characteristic as it occurs in nature as distinguished from mutant or variant forms.

### DETAILED DESCRIPTION OF THE INVENTION

The present disclosure provides cytosine base editors that comprise an evolutionary directed adenosine deaminase domain (e.g., a variant of an adenosine deaminase, TadA, that preferentially deaminates cytidine in DNA as described herein) and a napDNAbp domain (e.g., a Cas9 protein) capable of binding to a specific nucleotide sequence, wherein the adenosine deaminase variants provide the base editor (TadCBEs) with a smaller size and lower off-target effects while maintaining the high editing efficiencies of existing CBEs. The deamination of a cytidine by TadCBEs may lead to a point mutation from cytosine (C) to (T), a process referred to herein as nucleic acid editing, thus converting a C·G base pair to a T·A base pair. Such base editors are useful, inter alia, for targeted editing of nucleic acid sequences, such as DNA molecules. Such base editors may be used for targeted editing of DNA in vitro, e.g., for the generation of mutant cells or animals. Such base editors may be used for the introduction of targeted mutations in the cell of a living mammal. Such base editors may also be used for the introduction of targeted mutations for the correction of genetic defects in cells ex vivo, e.g., in cells obtained from a subject that are subsequently re-introduced into the same or another subject, or for multiplexed editing of multiple genes in a genome. And these base editors may be used for the introduction of targeted mutations in vivo, e.g., the correction of genetic defects or the introduction of deactivating mutations in disease-associated genes in a subject, or for multiplexed editing of a genome. The cytosine base editors described herein may be utilized for the targeted editing of T to C mutations (e.g., targeted genome editing). The invention provides deaminases, base editors, nucleic acids, vectors, cells, compositions, methods, kits, and uses that utilize the deaminases and base editors provided herein.

Here, PACE and PANCE were utilized to alter the substrate specificity of TadA-8e, resulting in a new class of selective cytidine deaminases (TadA-CDs) and cytosine base editors (FIG. 1A). To enable cytidine deamination, TadA-CD variants acquired mutations at residues that interact with the DNA backbone near the active site. The disclosed TadA-CD cytosine base editors (TadCBEs) are highly active and exhibit comparable or higher C·G-to-T·A conversion efficiencies compared to current BE4max, evoAPOBEC1-BE4max (evoA), and evoFERNY-BE4max (evoFERNY) CBEs across a variety of sites in mammalian cells. TadA-CDs are also compatible with both SpCas9 (PAM=NGG) and evolved eNme2-C Cas9 (PAM=N₄CN) variants, facilitating broad target accessibility. Off-target analysis reveals that TadCBEs induce lower Cas-independent off-target DNA and RNA editing than widely used APOBEC-based CBE variants. The addition of a V106W mutation^{9,34} further reduces off-target editing by TadCBEs, refines their editing window, and improves C·G-to-T·A selectivity, while preserving peak on-target editing efficiency. Herein, evolved TadCBEs are extensively characterized using a library of 10,638 genomically integrated, highly variable target sites in mouse embryonic stem cells (mESCs) to determine the selectivity and sequence context preferences of TadCBEs. TadA-CDs are also compatible with both SpCas9 and evolved eNme2-C Cas9 variants, facilitating broad target accessibility. The disclosed TadCBEs may be used for efficient cytosine base editing in human cells at therapeutically relevant loci, including multiplexed editing, and in particular for cytosine editing at a therapeutically relevant site in primary human hematopoietic stem and progenitor cells (HSPCs). These disclosed TadCBEs exhibit a more precise editing window with fewer bystander edits at, for instance, the CXCR5 and CCR5 genes in primary human T cells than existing CBEs. This disclosure provides new family of small CBEs with high on-target activity, well-defined editing windows that facilitate precise base editing, and low off-target activity and establishes the potential of adenosine deaminases to evolve into selective cytidine deaminases.

In some aspects, the present disclosure relates to a adenosine deaminase with targeted cytosine activity (e.g., TadA-CD). In some embodiments, the TadA-CD is evolved from an E. coli tRNA adenosine deaminase previously engineered to act on single stranded DNA (as opposed to RNA) for adenosine base editing applications (e.g., TadA-8e). Those of skill in the art will appreciate that PACE and PANCE methodologies can be used to introduce additional mutations into the TadA-8e domain that alter the substrate specificity of the enzyme to yield a TadA-CD. In some embodiments the TadA-CDs (e.g., mutated TadA-8e deaminases) comprise between 80% to 99.5% sequence homology with the parent TadA-8e. In some cases, the TadA-CD deaminases comprise mutations at E27, V28, and H96 and further comprise at least one mutation at a residue selected from R26, M61, Y73, I76, M151, Q154, and A158, relative to the parent TadA-8e.

In some embodiments, the TadA-CD variant has an enhanced selectivity and deamination activity for cytosine, relative to adenosine, compared to the parent TadA-8e variant. For example, in some embodiments, TadA-CD deaminases covert between 85% and 92% (depending on the variant type) C-T base pairs at the C₄ and C₅ positions of target sequences to T-A base pairs with less than 2% editing of adenine; whereas base editors comprising TadA-8e deaminases convert at ˜90% A-T base pairs at the A6 position of target sequence to G-C base pairs with less than 2% editing of C-G to T-A base pairs (see Example 2). This represents a greater than 3000-fold change in the cytosine versus adenine base editing capability of the TadA-CD versus TadA-8e variants.

In some aspects, the present disclosure relates to cytosine base editors (CBEs) comprising a nucleic acid programmable DNA binding protein (e.g., Cas9) domain fused to a TadA-CD deaminase with cytidine activity (e.g., TadCBEs). In some embodiments, the napDNAbp domain comprises a Cas homolog, paralog, ortholog, or analog. The napDNAbp domain may be selected from a Cas9, a Cas9n (e.g., SpCas9n), a dCas9, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, a Cas9-NG, an LbCas12a, an enAsCas12a, an SaCas9, an SaCas9-KKH, a circularly permuted Cas9, an Argonaute (Ago) domain, a SmacCas9, a Spy-macCas9, an SpCas9-VRQR, an SpCas9-NRRH, an SpaCas9-NRTH, an SpCas9-NRCH, an eNme2Cas9, an eNme2-C Cas9, an enCjCas9, a SauriCas9, a Cas9-NG-VRQR, and or a variant thereof. In certain embodiments, the napDNAbp domain comprises or is a Cas9 domain or a Cas12a domain derived from S. pyogenes or S. aureus. In some cases, the napDNAbp domain is a Nme2Cas9 domain derived from Neisseria meningitidis. In some embodiments, the napDNAbp domain comprises a nuclease dead Cas9 (dCas9) domain, a Cas9 nickase (nCas9) domain, or a nuclease active Cas9 domain. In some cases, the napDNAbp domain is CjCas9. In various embodiments, the napDNAbp domain is a nickase.

The disclosed CBEs exhibit low levels of undesired editing, such as low Cas9-independent off-target editing. The disclosed CBEs exhibit fewer insertions and/or deletions (indels) and undesired editing of RNA molecules, following their use in methods of editing target sequences in nucleic acids. The disclosed CBEs also exhibit editing efficiencies that exceed efficiencies of the most commonly used CBEs for several therapeutically relevant sites and cell types.

In some aspects, the TadA-CDs exhibit a narrower editing window than native cytosine base editors while maintaining comparable or higher maximal editing efficiencies. Taken together, the small size of TadCBEs, their compatibility with eNme2Cas9 (and eNme2-C Cas9), their more focused editing windows, and their high editing efficiencies and selectivity's for cytosine over adenine base editing demonstrated their suitability for a variety of precision cytosine base editing applications.

Other aspects of the disclosure relate to composition comprising the TadCBEs as described herein and one or more guide RNAs, e.g., a single-guide RNA (“sgRNA”). In addition, the disclosure provides for nucleic acid molecules encoding and/or expressing the TadCBEs as described herein, as well as expression vectors or constructs for expressing the TadCBEs described herein and/or a gRNA (e.g., AAV vectors), host cells comprising said nucleic acid molecules and expression vectors, and one or more gRNAs, and compositions for delivering and/or administering nucleic acid-based embodiments described herein. In particular, the disclosure provides improved methods of delivery of the disclosed base editors, e.g., to a subject. Delivery of the disclosed TadCBE variants as RNPs, rather than DNA plasmids, typically increases on-target:off-target DNA editing ratios. Delivery of the disclosed TadCBE variants as mRNA molecules (e.g., using electroporation) may increase editing efficiencies. CBEs with apparent on-target editing efficiencies in vivo of about 50% have been described in International Publication No. WO/2019/226953, published Nov. 28, 2019, and Komor et al., Sci. Adv. 2017; 3:eaao4774, each of which is incorporated herein by reference. The disclosed CBEs may exhibit higher on-target editing efficiencies for a target cytosine base.

Further provided herein are methods of contacting any of the disclosed TadCBEs with a nucleic acid molecule, e.g., a nucleic acid molecule (e.g., DNA) comprising a target sequence. In some embodiments of the disclosed methods, low off-target DNA and/or RNA editing effects are observed. In some embodiments, the nucleic acid molecule comprises a DNA, e.g., a single-stranded DNA or a double-stranded DNA. The target sequence of the nucleic acid molecule may comprise a target nucleobase pair containing a cytosine (C). The target sequence may be comprised within a genome, e.g., a human genome. The target sequence may comprise a sequence, e.g., a target sequence with point mutation, associated with a disease or disorder, such as sickle cell disease or HIV/AIDS. In other embodiments, the target nucleotide sequence is in the genome of a rodent, such as a mouse or a rat. In other embodiments, the target nucleotide sequence is in the genome of a domesticated animal, such as a horse, cat, dog, or rabbit. In some embodiments, the target nucleotide sequence is in the genome of a research animal. In some embodiments, the target nucleotide sequence is in the genome of a genetically engineered non-human subject. In some embodiments, the target nucleotide sequence is in the genome of a plant. In some embodiments, the target nucleotide sequence is in the genome of a microorganism, such as a bacteria.

Still further, the present disclosure provides for methods of generating the TadCBEs described herein, as well as methods of using the base editors or nucleic acid molecules encoding any of these base editors in applications including editing a nucleic acid molecule, e.g., a genome. In certain embodiments, methods of engineering the base editors provided herein involve a phage-assisted continuous evolution (PACE) system or non-continuous system (e.g., PANCE), which may be utilized to evolve one or more components of a base editor (e.g., a deaminase domain). In certain embodiments, following the successful evolution of one or more components of the base editor (e.g., a deaminase domain), methods of making the base editors comprise recombinant protein expression methodologies and techniques known to those of skill in the art. Exemplary base editors are made by fusing or associating the adenosine deaminase domain to any of a variety of napDNAbp domains disclosed herein, such as a Cas9 domain.

Without wishing to be bound by any particular theory, the TadCBEs described herein induce edits in nucleic acid substrates by use of TadA variants to deaminate C bases, causing C to T mutations via uracil formation. It is believed that fusing one or more uracil DNA glycosylase inhibitors to the deaminase and napDNAbp domains of the CBE inhibits innate DNA repair processes, which when coupled with a nucleic acid programmable DNA binding protein (e.g., dCas9) engineered to nick the non-edited DNA strand (e.g., the strand containing the G of the original C-G target base pair), results in conversion of the original C·G base pair to a T·A base pair. Without wishing to be bound by any particular theory, it is believed that mutations in residues 26-28 of the disclosed deaminases (relative to the TadA8e deaminase) facilitates the “sliding” of the backbone of the DNA substrate to enable the binding pocket of this adenosine deaminase to accept a cytosine.

In some embodiments, the TadCBEs described herein have been engineered to exhibit highly targeted and efficient editing capabilities. Such TadCBEs may be used, for example, to target and revert single nucleotide polymorphisms (SNPs) in disease-relevant genes, such as genes relevant to sickle cell disease and HIV/AIDS. In some cases, however, the TadCBEs described herein may permit substitution of a target C to a mixture of T, A, and G. For instance, TadCBEs lacking UGI domains may be useful, for example, as a screening platform for targeted random in vivo mutagenesis. More specifically, they can be used as forward genetic tool to screen for gain-of-function and/or loss-of-function variants at base resolution.

### Deaminase Domains

The disclosure provides cytidine base editors (TadCBEs) that have been evolved from an adenosine deaminase domain of an existing adenosine base editor (ABE). Adenosine deaminases used herein were evolved using standard methodologies to convert adenosine (A) to inosine (I) in mammalian DNA. Such adenosine deaminases may cause an A:T to G:C base pair conversion. The state-of-the-art ABE is ABE7.10, which is disclosed in International Publication No. WO 2018/027078, published Aug. 2, 2018. A more recently generated ABE is ABE8e, which contains an adenosine deaminase domain containing a single deaminase variant known as TadA8e, as described in International Publication No. WO 2021/158921, published Aug. 12, 2021. TadA8e contains nine mutations relative to TadA7.10, the adenosine deaminase of ABE7.10. TadA7.10 is also the deaminase domain of ABEmax, which is a variant of ABE7.10 that has been codon optimized for expression in human cells.

In some embodiments, the adenosine deaminases are variants of known adenosine deaminase TadA7.10, which comprises the following mutations as compared to wild-type ecTadA (SEQ ID NO: 325): W23R, H36L, P48A, R51L, L84F, A106V, D108N, H123Y, S146C, D147Y, R152P, E155V, I156F, and K157N. In some embodiments, the disclosed adenosine deaminases are variants of a TadA derived from a species other than E. coli, such as Staphylococcus aureus, Salmonella typhi, Shewanella putrefaciens, Haemophilus influenzae, Caulobacter crescentus, or Bacillus subtilis.

The substrate for the evolution experiments disclosed herein was TadA-8e, which contains the following mutations relative to TadA7.10: A109S, T111R, D119N, H122N, Y147D, F149Y, T166I, and D167N. Reference for disclosures of phage-assisted evolution experimental methods is made to International Publication No. WO 2018/027078; International Publication No. WO 2019/079347 published Apr. 25, 2019; International Publication No. WO 2019/226593, published Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2020/214842, published Oct. 22, 2020, and International Patent Application No. PCT/US2020/033873, filed May 20, 2020, International Publication No. WO 2020/236982, published Nov. 26, 2020, and International Publication No. WO 2021/158921, the contents of each of which are incorporated herein by reference in their entireties.

Exemplary, non-limiting, embodiments of adenosine deaminases used in the evolution are provided herein. In some embodiments, the adenosine deaminase domain of any of the disclosed base editors comprises a single adenosine deaminase, or a monomer. In some embodiments, the adenosine deaminase domain comprises 2, 3, 4 or 5 adenosine deaminases. In some embodiments, the adenosine deaminase domain comprises two adenosine deaminases, or a dimer. In some embodiments, the deaminase domain comprises a dimer of an engineered (or evolved) deaminase and a wild-type deaminase, such as a wild-type E. coli-derived deaminase. It should be appreciated that the mutations provided herein (e.g., mutations in ecTadA) may be applied to adenosine deaminases in other adenosine base editors, for example, those provided in International Publication No. WO 2018/027078, published Aug. 2, 2018; International Publication No. WO 2019/079347 on Apr. 25, 2019; International Application No PCT/US2019/033848, filed May 23, 2019, which published as International Publication No. WO 2019/226593 on Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, and International Patent Application No. PCT/US2020/28568, filed Apr. 16, 2020; all of which are incorporated herein by reference in their entireties.

Exemplary adenosine deaminase substrates that may be evolved into cytidine deaminases in accordance with the present disclosure are disclosed below. Exemplary TadA deaminases derived from Bacillus subtilis (set forth in full as SEQ ID NO: 318), S. aureus (SEQ ID NO: 317), and S. pyogenes (SEQ ID NO: 354) are provided. The amino acid substitutions in E. coli TadA-8e, and the homologous mutations in the B. subtilis, S. aureus, and S. pyogenes TadA deaminases, are shown. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is derived from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. One of skill in the art will be able to identify the corresponding residue in any homologous protein and in the respective encoding nucleic acid by methods well known in the art, e.g., by sequence alignment and determination of homologous residues.

In some embodiments, the adenosine deaminase substrate comprises TadA9, or a variant thereof. TadA9 contains V82S and Q154R substitutions relative to TadA-8e. (Stated differently, TadA9 contains Y147R, Q154R and I76Y mutations relative to TadA7.10.) In some embodiments, the adenosine deaminase comprises an amino acid sequence that is at least 80%, at least 85%, at least 90%, at least 95%, at least 96%, at least 97%, at least 98%, at least 99%, or at least 99.5% identical to the amino acid sequence of TadA9 (SEQ ID NO: 33). TadA9 may be referred to in the art as TadA*8.9. An ABE containing the TadA9 deaminase is referred to herein as ABE9. TadA9 is is described in additional detail in Gaudelli et al., Nat Biotechnol. 2020 July; 38(7):892-900 and PCT Publication No. WO 2021/050571, published Mar. 18, 2021, each of which are incorporated herein by reference.

In some embodiments, the adenosine deaminase substrate comprises TadA20, TadA-8.17-m (TadA17), or a variant thereof. TadA20 contains I76Y, V82S, Y123H, Y147R and Q154R substitutions relative to TadA7.10. TadA17 contains V82S and Q154R substitutions relative to TadA7.10. TadA20 and TadA17 are described in additional detail in Gaudelli et al., Nat Biotechnol. 2020 July; 38(7):892-900 and WO 2021/050571, published Mar. 18, 2021. TadA20 may be referred to in the art as TadA*8.20. In some embodiments, the adenosine deaminase comprises an amino acid sequence that is at least 80%, at least 85%, at least 90%, at least 95%, at least 96%, at least 97%, at least 98%, at least 99%, or at least 99.5% identical to the amino acid sequence of TadA20 (SEQ ID NO: 326). An ABE containing the TadA20 deaminase is referred to herein as ABE20. It may be referred to in the art as ABE8.20, ABE8.20-d, or ABE8.20-m. An ABE containing the TadA17 deaminase is referred to herein as ABE17. It may be referred to in the art as ABE8.17 or ABE8.17-m.

In some embodiments, the adenosine deaminase comprises an amino acid sequence that is at least 80%, at least 85%, at least 90%, at least 95%, at least 96%, at least 97%, at least 98%, at least 99%, or at least 99.5% identical to any of the amino acid sequences of SEQ ID NOs: 317-323.

In certain embodiments, the adenosine deaminase domain comprises an adenosine deaminase that has a sequence with at least 80%, at least 85%, at least 90%, at least 95%, at least 98%, at least 99%, or at least 99.5% sequence identity to one of the following:

TadA 7.10 (E. coli):
(SEQ ID NO: 315)
MSEVEFSHEYWMRHALTLAKRARDEREVPVGAVLVLNNRVIGEGWNRAIGLHDPT
AHAEIMALRQGGLVMQNYRLIDATLYVTFEPCVMCAGAMIHSRIGRVVFGVRNAK
TGAAGSLMDVLHYPGMNHRVEITEGILADECAALLCYFFRMPRQVFNAQKKAQSS
TD
TadA-8e (E. coli):
(SEQ ID NO: 350)
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKR
GAAGSLMNVLNYPGMNHRVEITEGILADECAALLCDFYRMPRQVFNAQKKAQSSI
N
Tad1:
(SEQ ID NO: 1)
SEVEFSHEYWMRHALTLAKRARDEGEVPVGAVLVLNNRVIGEGWNRAIGLYDPTA
HAEIMALRQGGLVMQNYRLIDATLYVTFEPCVMCAGAMIHSRIGRVVFGVRNSKR
GAAGSLMNVLNYPGMDHRVEITEGILADECAALLCDFYRMPRQVFNAQKKAQSSI
N
Tad2:
(SEQ ID NO: 2)
SEVEFSHEYWMRHALTLAKRARDEGEVPVGAVLVLNNRVIGEGWNRAIGLHDPTA
HAEIMALRQGGLVMQNYRLIDATLYVTFEPCVMCAGAMIHSRIGRVVFGVRNSKR
GAAGSLMNVLNYPGMDHRVEITEGILADECAALLCDFYRMPRQVFNAQKKAQSSI
N
Tad3:
(SEQ ID NO: 3)
SEVEFSHEYWMRHALTLAKRARDEREVPVGAVLVLNNRVIGEGWNRAIGLHDPTA
HAEIMALRQGGLVMQNYGLIDATLYVTFEPCVMCAGAIIHSRIGRVVFGVRNSKRG
AAGSLMNVLNYPGMNHRVEITEGILADECAALLCDFYRMPRQVFNAQKKAQSSIN
Tad4:
(SEQ ID NO: 4)
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKR
GAAGSLMNVLNYPGMDHRVEITEGILADECAALLCDFYRMPRQVFNAQKKAQSSI
N
Tad6:
(SEQ ID NO: 5)
SEVEFSHEYWMRHALTLAKRARDEGEVPVGAVLVLNNRVIGEGWNRAIGLYDPTA
HAEIMALRQGGLVMQNYGLIDATLYVTFEPCVMCAGAMIHSRIGRVVFGVRNSKR
GAAGSLMNVLNYPGMDHRVEITEGILADECAALLCDFYRMPRQVFNAQKKAQSSI
N
Tad6-SR:
(SEQ ID NO: 6)
SEVEFSHEYWMRHALTLAKRARDEGEVPVGAVLVLNNRVIGEGWNRAIGLYDPTA
HAEIMALRQGGLVMQNYGLIDATLYSTFEPCVMCAGAMIHSRIGRVVFGVRNSKR
GAAGSLMNVLNYPGMDHRVEITEGILADECAALLCDFYRMPRRVFNAQKKAQSSI
N
TadA9:
(SEQ ID NO: 33)
SEVEFSHEYWMRHALTLAKRARDEGEVPVGAVLVLNNRVIGEGWNRAIGLYDPTA
HAEIMALRQGGLVMQNYRLIDATLYVTFEPCVMCAGAMIHSRIGRVVFGVRNSKR
GAAGSLMNVLNYPGMDHRVEITEGILANECAALLCDFYRMPRQVFNAQKKAQSSI
N
TadA20
(SEQ ID NO: 326)
SEVEFSHEYWMRHALTLAKRARDEREVPVGAVLVLNNRVIGEGWNRAIGLHDPTA
HAEIMALRQGGLVMQNYRLYDATLYSTFEPCVMCAGAMIHSRIGRVVFGVRNAKT
GAAGSLMDVLHHPGMNHRVEITEGILADECAALLCRFFRMPRRVFNAQKKAQSSTD
Staphylococcus aureus TadA:
(SEQ ID NO: 317)
MGSHMTNDIYFMTLAIEEAKKAAQLGEVPIGAIITKDDEVIARAHNLRETLQQPTAH
AEHIAIERAAKVLGSWRLEGCTLYVTLEPCVMCAGTIVMSRIPRVVYGADDPKGGC
SGSLMNLLQQSNFNHRAIVDKGVLKEACSTLLTTFFKNLRANKKSTN
Bacillus subtilis TadA:
(SEQ ID NO: 318)
MTQDELYMKEAIKEAKKAEEKGEVPIGAVLVINGEIIARAHNLRETEQRSIAHAEML
VIDEACKALGTWRLEGATLYVTLEPCPMCAGAVVLSRVEKVVFGAFDPKGGCSGT
LMNLLQEERFNHQAEVVSGVLEEECGGMLSAFFRELRKKKKAARKNLSE
Salmonella typhimurium (S. typhimurium) TadA:
(SEQ ID NO: 319)
MPPAFITGVTSLSDVELDHEYWMRHALTLAKRAWDEREVPVGAVLVHNHRVIGEG
WNRPIGRHDPTAHAEIMALRQGGLVLQNYRLLDTTLYVTLEPCVMCAGAMVHSRI
GRVVFGARDAKTGAAGSLIDVLHHPGMNHRVEIIEGVLRDECATLLSDFFRMRRQE
IKALKKADRAEGAGPAV
Shewanella putrefaciens (S. putrefaciens) TadA:
(SEQ ID NO: 320)
MDEYWMQVAMQMAEKAEAAGEVPVGAVLVKDGQQIATGYNLSISQHDPTAHAEI
LCLRSAGKKLENYRLLDATLYITLEPCAMCAGAMVHSRIARVVYGARDEKTGAAG
TVVNLLQHPAFNHQVEVTSGVLAEACSAQLSRFFKRRRDEKKALKLAQRAQQGIE
Haemophilus influenzae F3031 (H. influenzae) TadA:
(SEQ ID NO: 321)
MDAAKVRSEFDEKMMRYALELADKAEALGEIPVGAVLVDDARNIIGEGWNLSIVQ
SDPTAHAEIIALRNGAKNIQNYRLLNSTLYVTLEPCTMCAGAILHSRIKRLVFGASDY
KTGAIGSRFHFFDDYKMNHTLEITSGVLAEECSQKLSTFFQKRREEKKIEKALLKSLS
DK
Caulobacter crescentus (C. crescentus) TadA:
(SEQ ID NO: 322)
MRTDESEDQDHRMMRLALDAARAAAEAGETPVGAVILDPSTGEVIATAGNGPIAA
HDPTAHAEIAAMRAAAAKLGNYRLTDLTLVVTLEPCAMCAGAISHARIGRVVFGA
DDPKGGAVVHGPKFFAQPTCHWRPEVTGGVLADESADLLRGFFRARRKAKI
Geobacter sulfurreducens (G. sulfurreducens) TadA:
(SEQ ID NO: 323)
MSSLKKTPIRDDAYWMGKAIREAAKAAARDEVPIGAVIVRDGAVIGRGHNLREGSN
DPSAHAEMIAIRQAARRSANWRLTGATLYVTLEPCLMCMGAIILARLERVVFGCYD
PKGAAGSLYDLSADPRLNHQVRLSPGVCQEECGTMLSDFFRDLRRRKKAKATPALF
IDERKVPPEP
Streptococcus pyogenes (S. pyogenes) TadA:
(SEQ ID NO: 354)
MPYSLEEQTYFMQEALKEAEKSLQKAEIPIGCVIVKDGEIIGRGHNAREESNQAIMH
AEIMAINEANAHEGNWRLLDTTLFVTIEPCVMCSGAIGLARIPHVIYGASNQKFGGA
DSLYQILTDERLNHRVQVERGLLAADCANIMQTFFRQGRERKKIAKHLIKEQSDPFD
Aquifex aeolicus (A. aeolicus) TadA:
(SEQ ID NO: 355)
MGKEYFLKVALREAKRAFEKGEVPVGAIIVKEGEIISKAHNSVEELKDPTAHAEML
AIKEACRRLNTKYLEGCELYVTLEPCIMCSYALVLSRIEKVIFSALDKKHGGVVSVF
NILDEPTLNHRVKWEYYPLEEASELLSEFFKKLRNNII

In some embodiments, the TadA deaminase is a full-length E. coli TadA deaminase (ecTadA). For example, in certain embodiments, the adenosine deaminase domain comprises a deaminase that comprises the amino acid sequence:

(SEQ ID NO: 325)
MRRAFITGVFFLSEVEFSHEYWMRHALTLAKRAWDEREVPVGAVLVHN
NRVIGEGWNRPIGRHDPTAHAEIMALRQGGLVMQNYRLIDATLYVTLE
PCVMCAGAMIHSRIGRVVFGARDAKTGAAGSLMDVLHHPGMNHRVEIT
EGILADECAALLSDFFRMRRQEIKAQKKAQSSTD

### TadA-Derived Cytidine Deaminases (TadA-CD)

Aspects of the disclosure relate to an evolved adenosine deaminase with enhanced cytosine specificity and cytidine deamination activity. The evolved deaminase, according to certain embodiments, is capable of deaminating a cytidine in DNA. In some embodiments, the deaminase is evolved from a parent adenosine deaminase using continuous and/or non-continuous laboratory-directed methods (e.g., PACE and PANCE). In some embodiments, the parent adenosine deaminase evolved using PACE and/or PANCE has cytidine deaminase activity. The deaminase of the present disclosure may be evolved from any adenosine deaminase reported to date to have adenosine deaminase activity, such as, for example, those described in International Patent Application No. PCT/US2017/045381, filed Aug. 3, 2017; International Patent Application No. PCT/US2020/028568, filed Apr. 16, 2020; International Patent Application No. PCT/US2021/016827, filed Feb. 5, 2021; PCT/US2022/073781, filed Jul. 15, 2022; all of which are incorporated herein by reference in their entireties. In some cases, the parent deaminase comprises an E. coli tRNA adenosine deaminase (TadA). The deaminase of the instant application may be evolved from a previously mutated (i.e., evolved) parent TadA variant, such as, for example, those described in International Patent Application No. PCT/US2021/016827, filed Feb. 5, 2021, which published as WO 2021/158921 on Aug. 12, 2021. For instance, in some embodiments, the parent adenosine deaminase is TadA7.10. In other embodiments, the parent adenosine deaminase is the TadA8e variant which contains an additional 8 mutations relative to TadA7.10: A109S, T111R, D119N, H122N, Y147D, F149Y, T166I, and D167N. Other parent adenosine deaminase substrates are also possible.

In some embodiments, the TadA-derived cytidine deaminase of the instant application is derived from a parent adenosine deaminase (e.g., TadA-8e) using a combination of phage-assisted continuous evolution (PACE) and non-continuous evolution (PANCE). The parent adenosine deaminase, according to certain embodiments, comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 41. In some cases, the parent adenosine deaminase comprises the sequence of SEQ ID NO: 41.

In some embodiments, the evolved TadA-derived cytidine deaminase are, at least partially, homologous to the parent TadA-8e variant. For instance, the TadA-derived cytidine deaminase (e.g., TadA-CD), according to certain embodiments, comprise an amino acid sequence that is at least 80% identical, at least 85% identical, at least 90% identical, at least 95% identical, at least 98% identical, at least 99% identical, and at least 99.5% identical to the amino acid sequence of SEQ ID NO: 41, wherein residue 27 of SEQ ID NO: 41 is any amino acid expect for E (glutamic acid). TadA-CDs with other sequence homologies are also possible. For example, in certain embodiments, the TadA-derived cytidine deaminase (e.g., TadA-CD) comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 41, wherein residue 28 of SEQ ID NO: 41 is any amino acid expect for V (valine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 41, wherein residue 96 of SEQ ID NO: 41 is any amino acid expect for H (histidine).

As will be appreciated by those of skill in the art, TadA-derived cytidine deaminases (e.g., TadA-CD) may comprise a plurality of mutations relative to the parent adenosine deaminase (e.g., TadA-8e). In some embodiments, the deaminase of the instant application (e.g., TadA-CD) comprises mutations at residues E27, V28, and H96. In some embodiments, the disclosed deaminase further comprises at least one mutation at a residue selected from R26, M61, Y73, I76, M151, Q154, and A158, in the amino acid sequence of SEQ ID NO: 41, or corresponding mutations in a homologous adenosine deaminase.

In some embodiments, the deaminase comprises at least one mutation selected from E27A, E27K, V28G, V28A, and H96N, and further comprises at least one mutation at a residue selected from R26G, M61I, Y73H, Y73S, Y73C, I76F, M151I, Q154R, Q154H, and A158S, in the amino acid sequence of SEQ ID NO: 41, or a corresponding mutation in a homologous adenosine deaminase. Other mutations are also possible. For example, in certain embodiments, the TadA-CD enzyme comprises mutations selected from E27A, V28G, and H96N, and further comprises at least one mutation selected from R26G, M61I, Y73H, Y73S, Y73C, I76F, M151I, Q154R, Q154H, and A158S, in the amino acid sequence of SEQ ID NO: 41, or corresponding mutations in a homologous adenosine deaminase.

Other exemplary embodiments may include (1) deaminases comprising mutations E27K, V28G, and H96N, and further comprising at least one mutation selected from R26G, M61I, Y73H, Y73S, Y73C, I76F, M151I, Q154R, Q154H, and A158S, in the amino acid sequence of SEQ ID NO: 41 or corresponding mutations in a homologous adenosine deaminase; (2) deaminases comprising mutations E27A, V28A, and H96N, and further comprising at least one mutation selected from R26G, M61I, Y73H, Y73S, Y73C, I76F, M151I, Q154R, Q154H, and A158S, in the amino acid sequence of SEQ ID NO: 41, or corresponding mutations in a homologous adenosine deaminase; (3) deaminases comprising mutations E27K, V28A, and H96N, and further comprising at least one mutation selected from R26G, M61I, Y73H, Y73S, Y73C, I76F, M151I, Q154R, Q154H, and A158S, in the amino acid sequence of SEQ ID NO: 41, or corresponding mutations in a homologous adenosine deaminase.

In some embodiments, the TadA-derived cytidine deaminases (TadA-CD) comprise at least two mutations at residues selected from R26, M61, Y73, I76, M151, Q154, and A158 (relative to the parent deaminase). In other embodiments, the TadA-CD comprises at least two mutations at residues selected from R26G, M61I, Y73H, I76F, M151I, Q154H, Q154R, and A158S.

In some aspects, TadA-derived cytidine deaminases are provided that may retain some A-to-G base editing activity. Without wishing to be bound to any particular theory, it has been determined via a reversion analysis that residues 26-28 of the TadA-8e deaminase (set forth in SEQ ID NO:41), which lie on a loop near the active site, are critical for switching selectivity for adenosine to cytidine. It is further believed that substrate positioning in the active site is a key determinant of deamination selectivity, and that the sequence context may influence selective deamination of the target base, as interactions between the TadA-CDs and the 5′ and 3′ may impact substrate positioning in the active site.

Again, without wishing to be bound by theory, it is believed that residual A-to-T editing is highest when the adenine is in the center of the editing window (e.g., protospacer position 5 or 6 for SpCas9, with PAM as position 21-23) and is preceded by T or C. In some embodiments, the addition of a V106W mutation improves the selectivity by suppressing A deamination to a greater extent than C deamination.

In some aspects, TadA-derived cytidine deaminases are provided that provide efficient conversions of target cytosines to thymines and target adenines to guanines (herein referred to as “TadA-dual” deaminases and base editors). TadA-dual deaminases are able to edit C and A bases within a protospacer, and in particular within the editing window of a protospacer. These editors install both A-to-G and C-to-T edits at roughly equivalent efficiencies.

For instance, the disclosed TadA dual deaminases install A-to-G edits and C-to-T edits at a ratio of roughly 1.1:1. In some embodiments, the dual editors provide A-to-G and C-to-T editing at a ratio of 0.7:1, 0.8:1, 0.9:1, 1:1, 1.1:1, 1.2:1, 1.3:1, 1.4:1, or 1.5:1. Other ranges are also possible, including ratios greater than 1.5:1. These evolved TadA deaminases, and the “dual” editors containing these deaminases, that are capable of editing A·T-to-G·C with virtually identical efficiency as C·G-to-T·A, may be useful for screening applications, such as methods of screening novel Cas homolog domains and other napDNAbp domains for editing activity against various target sequences. These deaminases, and dual editors, may further be useful for mutagenesis applications, such as in vivo forward genetic mutagenesis screens or targeted random mutagenesis screens. These dual editors may also be useful for multiplexed editing applications.

### Dual Editors

In some embodiments, a TadA-based dual editor comprises a cytidine deaminase comprising one, two, three, four, or five mutations selected from R26G, V28A, A48R, Y73S, and H96N. This dual editor is referred to herein as TadDE, and the dual-editing deaminase is referred to herein as TadA-CDf (e.g., TadA-Dual), which has an amino acid sequence set forth in SEQ ID NO: 39.

As such, in some embodiments, provided herein are deaminases that comprise mutations at residues R26, V28, A48, and Y73 in the amino acid sequence of SEQ ID NO: 41, or corresponding mutations in a homologous adenosine deaminase. Further provided herein are deaminases that comprise mutations at residues R26, E27, V28, A48, and Y73 (i.e., further comprise a mutation at E27) in the amino acid sequence of SEQ ID NO: 41. In particular embodiments, these deaminases comprise the mutations R26G, V28A, A48R, Y73S, and H96N. In some embodiments, these deaminases comprise the mutations R26G, V28G, A48R, and Y73C.

As described above and herein, preferred Tad-A-derived cytidine deaminases, evolved using PACE and PANCE approaches, may comprise one or more mutations. For instance, TadA-CD variants may comprise at least one mutation selected from R26G, E27A, V28G, I76F, H96N, and M151I (e.g, TadA-CDa, SEQ ID NO: 34); R26G, E27A, V28G, I76F, H96N, and A158S (e.g, TadA-CDb, SEQ ID NO: 35); R26G, E27A, V28G, I76F, H96N, Q154R, and A158S (e.g, TadA-CDc, SEQ ID NO: 36); E27A, V28G, Y73H, H96N, Q154H, and A158S (e.g., TadA-CDd, SEQ ID NO: 37); R26G, V28A, A48R, Y73S, and H96N (e.g., TadA-CDe, SEQ ID NO: 38); V28A, A48R, and Y73S (e.g, TadA-CDf, SEQ ID NO: 39), and R26G, V28G, A48R, and Y73C (e.g, TadA-CDg, SEQ ID NO: 40).

In some preferred embodiments, the deaminase comprises the mutations R26G, E27A, V28G, I76F, H96N, and A158S (e.g., TadA-CDa, SEQ ID NO: 34), R26G, E27A, V28G, I76F, H96N, Q154R, and A158S (e.g., TadA-CDb, SEQ ID NO: 35), R26G, E27A, V28G, I76F, H96N, and M151I (e.g., TadA-CDc, SEQ ID NO: 36), E27K, V28A, M61I, and H96N (e.g., TadA-CDd, SEQ ID NO: 37), E27A, V28G, Y73H, H96N, Q154H, and A158S (e.g., TadA-CDe, SEQ ID NO: 38), R26G, V28A, A48R, Y73S, and H96N (e.g., TadA-CDf, SEQ ID NO: 39), and R26G, V28G, A48R, and Y73C (e.g., TadA-CDg, SEQ ID NO: 40).

Those of ordinary skill in the art will understand that the evolved deaminases described herein may, because of the varying types and combinations of inherited mutations, exhibit varying specificities and/or deamination activities toward cytosine and/or adenosine bases. In some embodiments, the cytidine deamination activity of the TadA-CD exceeds the cytidine deamination activity of TadA-8e. For instance, the cytidine deamination activity of the TadA-CD variant may be greater than or equal 10×, greater than or equal 20×, greater than or equal 40×, greater than or equal 80×, greater than or equal 100×, greater than or equal 200×, greater than or equal 400×, greater than or equal 800×, greater than or equal 1000×, greater than or equal 2000×, greater than or equal 3000×, greater than or equal 4000× the cytidine deamination activity of TadA-8e. In other embodiments, the cytidine deamination activity of the TadA-CD variant is less than or equal to 4000×, is less than or equal to 2000×, is less than or equal to 1000×, is less than or equal to 800×, is less than or equal to 800×, is less than or equal to 400×, is less than or equal to 200×, is less than or equal to 100×, is less than or equal to 80×, is less than or equal to 40×, is less than or equal to 20×, or is less than or equal to 10× the cytidine deamination activity of TadA-8e.

In some embodiments, the adenosine deamination activity of the TadA-CD deaminase is less than the deaminase activity of TadA-8e. For instance, in some cases the adenosine deamination activity of the TadA-CD variant is less than or equal to 4000×, is less than or equal to 2000×, is less than or equal to 1000×, is less than or equal to 800×, is less than or equal to 800×, is less than or equal to 400×, is less than or equal to 200×, is less than or equal to 100×, is less than or equal to 80×, is less than or equal to 40×, is less than or equal to 20×, or is less than or equal to 10× the adenosine deamination activity of TadA-8e.

In some embodiments, the TadA-CD variants described above and herein may also comprises a V106W mutation. It has recently been discovered that adenosine deaminase TadA variants comprising a V106W mutation, such as those described in International Patent Publication Nos. WO 2021/214842 and WO 2021/158921, each of which is incorporated herein by reference, had reduced Cas-independent off-target editing of DNA and RNA while maintaining high levels of on-target adenosine deaminase activity. In some embodiments, TadA-CD variants comprising the V106W mutation average greater than or equal to 50%, greater than or equal to 60%, greater than or equal to 70%, greater than or equal to 80%, and greater than or equal to 90% peak editing efficiencies. In other embodiments, TadA-CD variants comprising the V106W mutation average less than or equal to 90%, less than or equal to 80%, less than or equal to 70%, less than or equal to 60%, or less than or equal to 50% peak editing efficiencies. ABEs containing only a single TadA deaminase domain, rather than a single-chain dimer, allow for reduction in editor size^{30,31}. Moreover, while SaCas9 is small enough (1053 amino acids in length, SEQ ID NO: 347) to provide a single AAV-compatible base editor, its utility is greatly limited by the rarity of its NNGRRT PAM. Since base editing requires the presence of a suitable PAM to place the target nucleotide within the editing window, TadCBEs that collectively offer broad PAM compatibility along with simple and efficient in vivo delivery would advance in vivo applications of base editing.

In some embodiments, any one of the deaminases listed in Table 10 may further comprises a V106W mutation. In some embodiments, the TadA-CD variants comprise at least 80%, at least 85%, at least 90%, at least 95%, at least 98%, at least 99%, or at least 99.5% to any of the amino acid sequences listed in Table 10, wherein anyone of the sequences listed in Table 10 further comprise a V106W mutation.

In some embodiments, the TadA variants comprise at least 80%, 85%, 90%, 95%, 98%, 99%, or 99.5% identity to any of the amino acid sequences listed in Table 10.

TABLE 10

| TadA | | SEQ ID |
|---|---|---|
| Variant | Sequence | NO: |
| TadA-CDa | MSEVEFSHEYWMRHALTLAKRARDEGAGPVGAVLVLNNRVIGEGWNR | 34 |
| | AIGLHDPTAHAEIMALRQGGLVMQNYRLFDATLYVTFEPCVMCAGAMIN | |
| | SRIGRVVFGVRNSKRGAAGSLMNVLNYPGMNHRVEITEGILADECAALLC | |
| | DFYRMPRQVFNSQKKAQSSIN | |
| TadA-CDb | MSEVEFSHEYWMRHALTLAKRARDEGAGPVGAVLVLNNRVIGEGWNR | 35 |
| | AIGLHDPTAHAEIMALRQGGLVMQNYRLFDATLYVTFEPCVMCAGAMIN | |
| | SRIGRVVFGVRNSKRGAAGSLMNVLNYPGMNHRVEITEGILADECAALLC | |
| | DFYRMPRRVFNSQKKAQSSIN | |
| TadA-CDc | MSEVEFSHEYWMRHALTLAKRARDEGAGPVGAVLVLNNRVIGEGWNR | 36 |
| | AIGLHDPTAHAEIMALRQGGLVMQNYRLFDATLYVTFEPCVMCAGAMIN | |
| | SRIGRVVFGVRNSKRGAAGSLMNVLNYPGMNHRVEITEGILADECAALLC | |
| | DFYRIPRQVFNSQKKAQSSIN | |
| TadA-CDd | MSEVEFSHEYWMRHALTLAKRARDERKAPVGAVLVLNNRVIGEGWNRA | 37 |
| | IGLHDPTAHAEIIALRQGGLVMQNYRLIDATLYVTFEPCVMCAGAMINSRI | |
| | GRVVFGVRNSKRGAAGSLMNVLNYPGMNHRVEITEGILADECAALLCDF | |
| | YRMPRQVFNAQKKAQSSIN | |
| TadA-CDe | MSEVEFSHEYWMRHALTLAKRARDERAGPVGAVLVLNNRVIGEGWNRA | 38 |
| | IGLHDPTAHAEIMALRQGGLVMQNHRLIDATLYVTFEPCVMCAGAMINS | |
| | RIGRVVFGVRNSKRGAAGSLMNVLNYPGMNHRVEITEGILADECAALLCD | |
| | FYRMPRHVFNSQKKAQSSIN | |
| TadA-CDf | MSEVEFSHEYWMRHALTLAKRARDEGEAPVGAVLVLNNRVIGEGWNRR | 39 |
| (e.g,. TadA- | IGLHDPTAHAEIMALRQGGLVMQNSRLIDATLYVTFEPCVMCAGAMINS | |
| Dual) | RIGRVVFGVRNSKRGAAGSLMNVLNYPGMNHRVEITEGILADECAALLCD | |
| | FYRMPRHVFNAQKKAQSSIN | |
| TadA-CDg | MSEVEFSHEYWMRHALTLAKRARDEGEGPVGAVLVLNNRVIGEGWNRR | 40 |
| | IGLHDPTAHAEIMALRQGGLVMQNCRLIDATLYVTFEPCVMCAGAMIHS | |
| | RIGRVVFGVRNSKRGAAGSLMNVLNYPGMNHRVEITEGILADECAALLCD | |
| | FYRMPRQVFNAQKKAQSSIN | |
| TadA-8e | MSEVEFSHEYWMRHALTLAKRARDEREVPVGAVLVLNNRVIGEGWNRA | 41 |
| (substrate) | IGLHDPTAHAEIMALRQGGLVMQNYRLIDATLYVTFEPCVMCAGAMIHS | |
| | RIGRVVFGVRNSKRGAAGSLMNVLNYPGMNHRVEITEGILADECAALLCD | |
| | FYRMPRQVFNAQKKAQSSIN | |

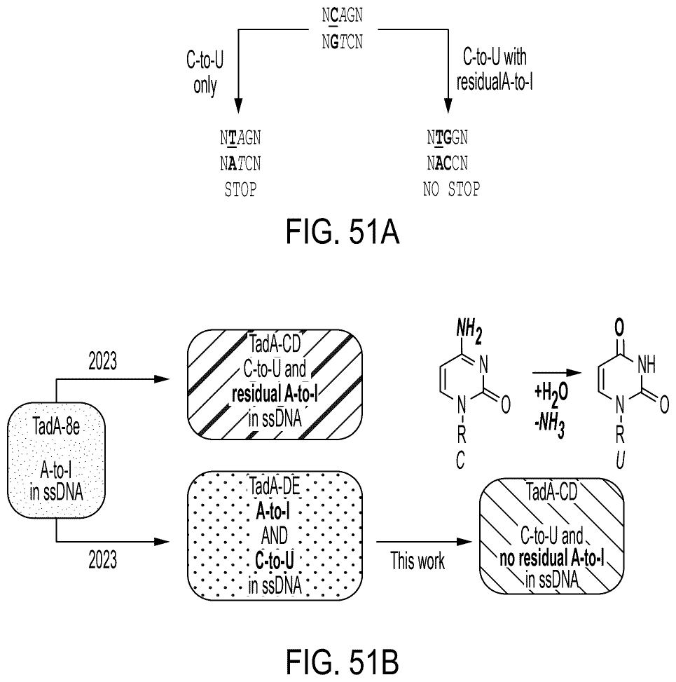

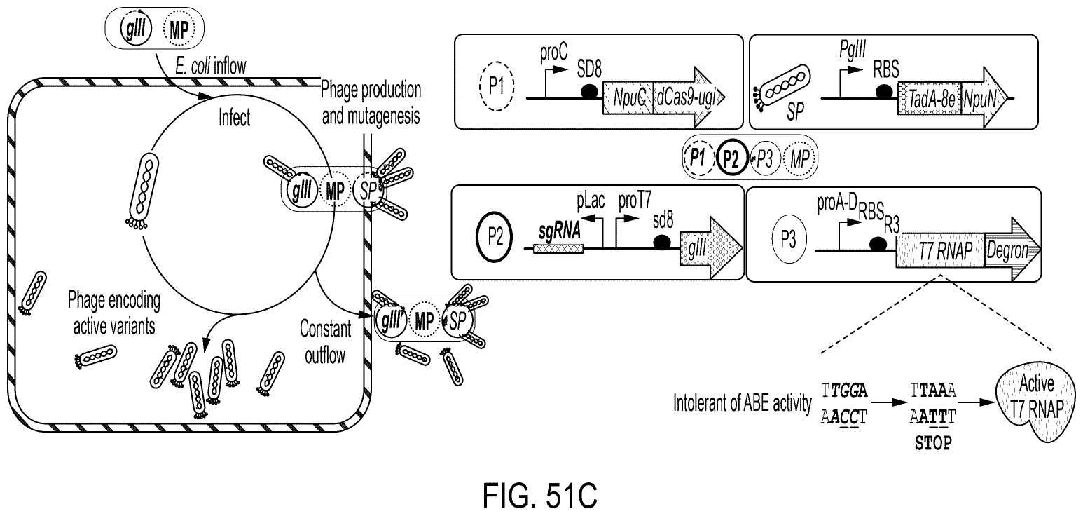

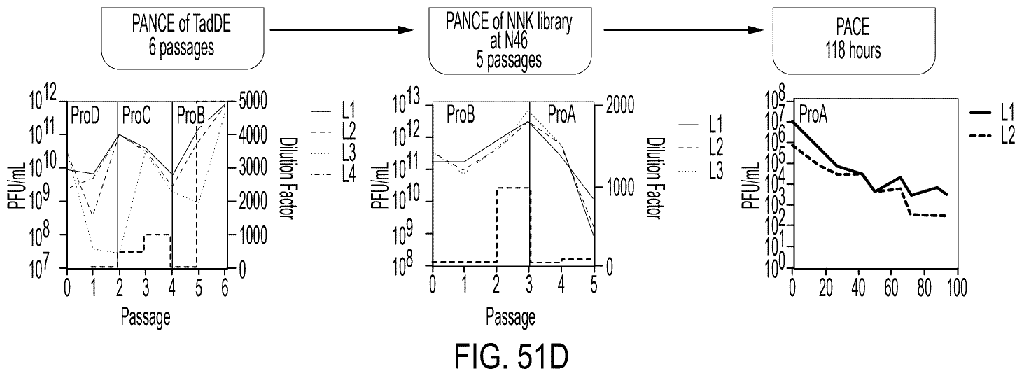

In some embodiments, the dual editor deaminase (e.g., TadA-CDf or TadA-Dual, SEQ ID NO: 39) of the TadDE dual editor may be further evolved, for example, using the PACE and/or PANCE assays described further below and elsewhere herein. In some embodiments, the TadA-Dual deaminase (e.gl, TadA-CDf, SEQ ID NO: 39) is further evolved to enhance specificity toward cytosine bases and reduce specificity toward adenosine bases. For example, FIG. 51E shows a table listing evolved TadA-Dual deaminases (e.g., TadDE-1 through TadDE-5) with their mutations relative to the unmutated TadA-Dual deaminase and its parent TadA-8e deaminase.

In some embodiments, the TadA-Dual deaminase is mutated using PACE as shown in FIG. 51C. In some embodiments, phage-assisted continuous evolution, or PACE (FIG. 51C, left) is used on conjugation with a selection circuit (FIG. 51C, right). In some embodiments, a continuous flow of E. coli host cells are infected by a selection phage encoding a partial deaminase (SP). Those of skill in the art will understand, that the E. coli host cells must also contain the plasmids that define the selection circuit as well as a mutagenesis plasmid. In the selection circuit, phage propagation is linked with the expression of gIII (P2), which can only be transcribed with active T7 RNA polymerase. In some embodiments, the T7 RNA polymerase (P3) is fused to a C-terminal degron, and the deaminase must perform C-to-U editing to install a stop codon before the degron, yielding active T7 RNA polymerase. In the event of phage infection, the full deaminase is completed using a split-intein system (P1) and mutations can occur on the deaminase. Beneficial mutations lead to phage propagation and enrichment in the lagoon, while the less-fit phage are unable to propagate and are subsequently washed out by the constant outflow.

In some embodiments, the TadA-Dual deaminase is mutated using phage-assisted non-continuous evolution (PANCE) as shown in FIG. 51D. In some embodiments, PANCE is performed on the TadA-Dual deaminase (SEQ ID NO: 39) until phage titers increase despite higher stringency from dilution factor and promoter strength, indicating that beneficial mutations have occurred. In some embodiments, the beneficial mutations comprise a mutation at position N46 in the deaminase.

In some embodiments, PANCE is performed on an NNK library at position N46 to further identify beneficial mutations. In some embodiments, combinations as mutagenesis assays may be performed. For example, in some embodiments, PACE is performed for more than 100 hours on resulting variants from PANCE studies.

In some embodiments, the evolved TadA-Dual deaminase comprises the mutations R26G, V28A, N46I, A48R, Y73P, and H96N (TadA-CD-1, FIG. 51E, PANCE, SEQ ID NO: 42) relative to the amino acid sequence of SEQ ID NO: 41. In some embodiments, the evolved TadA-Dual deaminase comprises the mutations R26G, V28A, N46T, A48R, Y73P, and H96N (TadA-CD-2, FIG. 51E, PANCE, SEQ ID NO: 43) relative to the amino acid sequence of SEQ ID NO: 41. In some embodiments, the evolved TadA-Dual deaminase comprises the mutations R26G, V28A, N46T, A48R, Y73S, and H96N (TadA-CD-3, FIG. 51E, PANCE, SEQ ID NO: 44) relative to the amino acid sequence of SEQ ID NO: 41. In some embodiments, the evolved TadA-Dual deaminase comprises the mutations R26G, V28A, N46V, A48R, Y73S, and H96N (TadA-CD-4, PANCE on NNK library at N46, SEQ ID NO:45) relative to the amino acid sequence of SEQ ID NO: 41. In some embodiments, the evolved TadA-Dual deaminase comprises the mutations R26G, V28A, N46V, A48R, Y73P, and H96N (TadA-CD-5, PANCE on NNK library at N46, SEQ ID NO: 46) relative to the amino acid sequence of SEQ ID NO: 41. In some embodiments, the evolved TadA-Dual deaminase comprises the mutations R26G, V28A, N46L, A48R, Y73P, and H96N (TadA-CD-6, PANCE on NNK library at N46, SEQ ID NO: 47) relative to the amino acid sequence of SEQ ID NO: 41. In some embodiments, the evolved TadA-Dual deaminase comprises the mutations V28A, N46L, A48P, and Y73P (TadA-CD-7, PANCE on NNK library at N46, SEQ ID NO: 48) relative to the amino acid sequence of SEQ ID NO: 41. In some embodiments, the evolved TadA-Dual deaminase comprises the mutations V28A, N46C, A48P, and Y73P (TadA-CD-8, PANCE on NNK library at N46, SEQ ID NO: 49) relative to the amino acid sequence of SEQ ID NO: 41. In some embodiments, the evolved TadA-Dual deaminase comprises the mutations R26G, V28A, N46V, A48R, Y73P, and H96N (TadA-CD-9, FIG. 51E, PACE, SEQ ID NO: 50) relative to the amino acid sequence of SEQ ID NO: 41. In some embodiments, the evolved TadA-Dual deaminase comprises the mutations R26G, V28A, N46V, A48R, Q71H, Y73P, and H96N (TadA-CD-10, FIG. 51E, PACE, SEQ ID NO: 51) relative to the amino acid sequence of SEQ ID NO: 41. In some embodiments, the evolved TadA-Dual deaminase comprises the mutations R26G, V28A, N46L, A48R, Y73P, and H96N (TadA-CD-11, FIG. 51E, PACE, SEQ ID NO: 52) relative to the amino acid sequence of SEQ ID NO: 41. In some embodiments, the evolved TadA-Dual deaminase comprises the mutations R26G, V28A, N46C, A48R, Y73P, and H96N (TadA-CD-12, FIG. 51E, PACE, SEQ ID NO: 53) relative to the amino acid sequence of SEQ ID NO: 41. In some embodiments, the evolved TadA-Dual deaminase comprises the mutations R26G, V28A, N46C, A48R, Y73P, H96N, and A162V (TadA-CD-13, FIG. 51E, PACE, SEQ ID NO: 54) relative to the amino acid sequence of SEQ ID NO: 41.

In some embodiments, the evolved TadA-Dual deaminase comprises the mutations R26G, V28A, N46I, A48R, Y73S, and H96N (TadA-CD-14, FIG. 51E, PANCE, SEQ ID NO: 359) relative to the amino acid sequence of SEQ ID NO: 41. In some embodiments, the evolved TadA-Dual deaminase comprises the mutations R26G, V28A, A48R, Q71S, Y73S, and H96N (TadA-CD-15, FIG. 51E, PANCE, SEQ ID NO: 360) relative to the amino acid sequence of SEQ ID NO: 41. In some embodiments, the evolved TadA-Dual deaminase comprises the mutations R26G, V28A, N46L, A48R, and Y73P (TadA-CD-16, FIG. 51E, PANCE, SEQ ID NO: 361) relative to the amino acid sequence of SEQ ID NO: 41. In some embodiments, the evolved TadA-Dual deaminase comprises the mutations R26G, V28A, N46L, A48R, Y73P, and H96N (TadA-CD-17, FIG. 51E, PANCE, SEQ ID NO: 362) relative to the amino acid sequence of SEQ ID NO: 41. In some embodiments, the evolved TadA-Dual deaminase comprises the mutations R26G, V28A, Y73P, and H96N (TadA-CD-18, FIG. 51E, PANCE, SEQ ID NO: 363) relative to the amino acid sequence of SEQ ID NO: 41. In some embodiments, the evolved TadA-Dual deaminase comprises the mutations R26G, V28A, N46V, A48R, Y73S, and H96N (TadA-CD-19, FIG. 51E, PANCE, SEQ ID NO: 364) relative to the amino acid sequence of SEQ ID NO: 41. In some embodiments, the evolved TadA-Dual deaminase comprises the mutations R26G, V28A, N46V, A48R, Y73P, and H96N (TadA-CD-20, FIG. 51E, PANCE, SEQ ID NO: 365) relative to the amino acid sequence of SEQ ID NO: 41. In some embodiments, the evolved TadA-Dual deaminase comprises the mutations R26G and N46L (TadA-CD-21, FIG. 51E, PANCE, SEQ ID NO: 366) relative to the amino acid sequence of SEQ ID NO: 41. In some embodiments, the evolved TadA-Dual deaminase comprises the mutations R26G, V28A, N46I, A48R, Y73P, and H96N (TadA-CD-22, FIG. 51E, PANCE, SEQ ID NO: 367) relative to the amino acid sequence of SEQ ID NO: 41. In some embodiments, the evolved TadA-Dual deaminase comprises the mutations R26G, V28A, N46V, A48R, Y73P, and H96N (TadA-CD-23, FIG. 51E, PANCE, SEQ ID NO: 368) relative to the amino acid sequence of SEQ ID NO: 41. In some embodiments, the evolved TadA-Dual deaminase comprises the mutations R26G, V28A, A48P, Y73H, T79P, and H96N (TadA-CD-24, FIG. 51E, PANCE, SEQ ID NO: 369) relative to the amino acid sequence of SEQ ID NO: 41. In some embodiments, the evolved TadA-Dual deaminase comprises the mutations R26G, N46I, and H96N (TadA-CD-25, FIG. 51E, PANCE, SEQ ID NO: 370) relative to the amino acid sequence of SEQ ID NO: 41.

In some embodiments, the evolved TadA-Dual deaminase comprises the mutations R26G, V28A, N46V, A48R, Y73P, and H96N (TadA-CD-26, FIG. 51E, PANCE, SEQ ID NO: 371) relative to the amino acid sequence of SEQ ID NO: 41. In some embodiments, the evolved TadA-Dual deaminase comprises the mutations R26G, V28A, N46L, A48R, Y73S, and H96N (TadA-CD-27, FIG. 51E, PANCE, SEQ ID NO: 372) relative to the amino acid sequence of SEQ ID NO: 41. In some embodiments, the evolved TadA-Dual deaminase comprises the mutations R26G, V28A, N46C, A48R, H96N, and A162V (TadA-CD-28, FIG. 51E, PANCE, SEQ ID NO: 373) relative to the amino acid sequence of SEQ ID NO: 41. In some embodiments, the evolved TadA-Dual deaminase comprises the mutations R26G, V28A, N46V, A48R, Q71H, Y73P, and H96N (TadA-CD-29, FIG. 51E, PANCE, SEQ ID NO: 374) relative to the amino acid sequence of SEQ ID NO: 41. In some embodiments, the evolved TadA-Dual deaminase comprises the mutations R26G, V28A, N46C, A48R, Y73P, and H96N (TadA-CD-30, FIG. 51E, PANCE, SEQ ID NO: 375) relative to the amino acid sequence of SEQ ID NO: 41. In some embodiments, the evolved TadA-Dual deaminase comprises the mutations R26G, V28A, N46C, A48R, Y73P, H96N, and A162V (TadA-CD-31, FIG. 51E, PANCE, SEQ ID NO: 376) relative to the amino acid sequence of SEQ ID NO: 41.

In some embodiments, the evolved TadA-Dual deaminase comprises the mutations R26G, V28A, N46V, A48R, Y73P, and H96N (TadA-CD-32, FIG. 51E, PANCE, SEQ ID NO: 377) relative to the amino acid sequence of SEQ ID NO: 41. In some embodiments, the evolved TadA-Dual deaminase comprises the mutations R26G, V28A, N46V, A48R, Y73S, and H96N (TadA-CD-33, FIG. 51E, PANCE, SEQ ID NO: 378) relative to the amino acid sequence of SEQ ID NO: 41. In some embodiments, the evolved TadA-Dual deaminase comprises the mutations R26G, V28A, N46V, A48P, Y73S, and H96N (TadA-CD-34, FIG. 51E, PANCE, SEQ ID NO: 379) relative to the amino acid sequence of SEQ ID NO: 41. In some embodiments, the evolved TadA-Dual deaminase comprises the mutations R26G, V28A, N46C, A48R, Y73P, and H96N (TadA-CD-35, FIG. 51E, PANCE, SEQ ID NO: 380) relative to the amino acid sequence of SEQ ID NO: 41. In some embodiments, the evolved TadA-Dual deaminase comprises the mutations R26G, V28A, L34M, N46L, A48R, Y73P, and H96N (TadA-CD-36, FIG. 51E, PANCE, SEQ ID NO: 381) relative to the amino acid sequence of SEQ ID NO: 41. In some embodiments, the evolved TadA-Dual deaminase comprises the mutations R26G, V28A, N46L, A48R, Y73P, and H96N (TadA-CD-37, FIG. 51E, PANCE, SEQ ID NO: 382) relative to the amino acid sequence of SEQ ID NO: 41. In some embodiments, the evolved TadA-Dual deaminase comprises the mutations R26G, V28A, N46L, A48P, R64K, Y73P, and H96N (TadA-CD-38, FIG. 51E, PANCE, SEQ ID NO: 383) relative to the amino acid sequence of SEQ ID NO: 41.

In some embodiments, the evolved TadA-Dual deaminase comprises the mutations N46I, S73P, and H154Q (TadA-CD-1, FIG. 51E, PANCE, SEQ ID NO: 42) relative to the amino acid sequence of SEQ ID NO: 39. In some embodiments, the evolved TadA-Dual deaminase comprises the mutations N46T (TadA-CD-2, FIG. 51E, PANCE, SEQ ID NO: 43) relative to the amino acid sequence of SEQ ID NO: 39. In some embodiments, the evolved TadA-Dual deaminase comprises the mutations N46T and H154Q (TadA-CD-3, FIG. 51E, PANCE, SEQ ID NO: 44) relative to the amino acid sequence of SEQ ID NO: 39. In some embodiments, the evolved TadA-Dual deaminase comprises the mutations N46V and H154Q (TadA-CD-4, PANCE on NNK library at N46, SEQ ID NO:45) relative to the amino acid sequence of SEQ ID NO: 39. In some embodiments, the evolved TadA-Dual deaminase comprises the mutations N46V, S73P, G105S, and H154Q (TadA-CD-5, PANCE on NNK library at N46, SEQ ID NO: 46) relative to the amino acid sequence of SEQ ID NO: 39. In some embodiments, the evolved TadA-Dual deaminase comprises the mutations N46L, S73P, and H154Q (TadA-CD-6, PANCE on NNK library at N46, SEQ ID NO: 47) relative to the amino acid sequence of SEQ ID NO: 39. In some embodiments, the evolved TadA-Dual deaminase comprises the mutations G26R N46L, R48P, S73P, N96H, and H154Q (TadA-CD-7, PANCE on NNK library at N46, SEQ ID NO: 48) relative to the amino acid sequence of SEQ ID NO: 39. In some embodiments, the evolved TadA-Dual deaminase comprises the mutations N46C, N96H, and H154Q (TadA-CD-8, PANCE on NNK library at N46, SEQ ID NO: 49) relative to the amino acid sequence of SEQ ID NO: 39. In some embodiments, the evolved TadA-Dual deaminase comprises the mutations N46V, S73P, and H154Q (TadA-CD-9, FIG. 51E, PACE, SEQ ID NO: 50) relative to the amino acid sequence of SEQ ID NO: 39. In some embodiments, the evolved TadA-Dual deaminase comprises the mutations N46V, Q71H, S73P, and H154Q (TadA-CD-10, FIG. 51E, PACE, SEQ ID NO: 51) relative to the amino acid sequence of SEQ ID NO: 39. In some embodiments, the evolved TadA-Dual deaminase comprises the mutations N46L and H154Q (TadA-CD-11, FIG. 51E, PACE, SEQ ID NO: 52) relative to the amino acid sequence of SEQ ID NO: 39. In some embodiments, the evolved TadA-Dual deaminase comprises the mutations N46C, S73P, and H154Q (TadA-CD-12, FIG. 51E, PACE, SEQ ID NO: 53) relative to the amino acid sequence of SEQ ID NO: 39. In some embodiments, the evolved TadA-Dual deaminase comprises the mutations N46C, S73P, H154Q, and A162V (TadA-CD-13, FIG. 51E, PACE, SEQ ID NO: 54) relative to the amino acid sequence of SEQ ID NO: 39.

In some embodiments, the evolved TadA-Dual deaminase comprises the mutations N46I and H154Q (TadA-CD-14, FIG. 51E, PACE, SEQ ID NO: 359) relative to the amino acid sequence of SEQ ID NO: 39.

In some embodiments, the evolved TadA-Dual deaminase comprises the mutations Q71S and H154Q (TadA-CD-15, FIG. 51E, PANCE, SEQ ID NO: 360) relative to the amino acid sequence of SEQ ID NO: 41. In some embodiments, the evolved TadA-Dual deaminase comprises the mutations N46L, S73P, N79T, and N96H (TadA-CD-16, FIG. 51E, PANCE, SEQ ID NO: 361) relative to the amino acid sequence of SEQ ID NO: 39. In some embodiments, the evolved TadA-Dual deaminase comprises the mutations N46L, S73P, N79T (TadA-CD-17, FIG. 51E, PANCE, SEQ ID NO: 362) relative to the amino acid sequence of SEQ ID NO: 39. In some embodiments, the evolved TadA-Dual deaminase comprises the mutations R48A, S73P, and N79T (TadA-CD-18, FIG. 51E, PANCE, SEQ ID NO: 363) relative to the amino acid sequence of SEQ ID NO: 39. In some embodiments, the evolved TadA-Dual deaminase comprises the mutations N46V and N79T (TadA-CD-19, FIG. 51E, PANCE, SEQ ID NO: 364) relative to the amino acid sequence of SEQ ID NO: 39. In some embodiments, the evolved TadA-Dual deaminase comprises the mutations N46V, S73P, and N79T (TadA-CD-20, FIG. 51E, PANCE, SEQ ID NO: 365) relative to the amino acid sequence of SEQ ID NO: 39. In some embodiments, the evolved TadA-Dual deaminase comprises the mutations A28V, N46L, R48A, S73Y, N79T, and N96H (TadA-CD-21, FIG. 51E, PANCE, SEQ ID NO: 366) relative to the amino acid sequence of SEQ ID NO: 39. In some embodiments, the evolved TadA-Dual deaminase comprises the mutations N46I, S73P, and N79T (TadA-CD-22, FIG. 51E, PANCE, SEQ ID NO: 367) relative to the amino acid sequence of SEQ ID NO: 39. In some embodiments, the evolved TadA-Dual deaminase comprises the mutations N46V, S73P, N79T, and G106S (TadA-CD-23, FIG. 51E, PANCE, SEQ ID NO: 368) relative to the amino acid sequence of SEQ ID NO: 39. In some embodiments, the evolved TadA-Dual deaminase comprises the mutations R48P, S73H, and N79P (TadA-CD-24, FIG. 51E, PANCE, SEQ ID NO: 369) relative to the amino acid sequence of SEQ ID NO: 39. In some embodiments, the evolved TadA-Dual deaminase comprises the mutations A28V, N46I, R48A, S73Y, and N79T (TadA-CD-25, FIG. 51E, PANCE, SEQ ID NO: 370) relative to the amino acid sequence of SEQ ID NO: 39.

In some embodiments, the evolved TadA-Dual deaminase comprises the mutations N46V and S73P (TadA-CD-26, FIG. 51E, PANCE, SEQ ID NO: 371) relative to the amino acid sequence of SEQ ID NO: 39. In some embodiments, the evolved TadA-Dual deaminase comprises the mutation N46L (TadA-CD-27, FIG. 51E, PANCE, SEQ ID NO: 372) relative to the amino acid sequence of SEQ ID NO: 39. In some embodiments, the evolved TadA-Dual deaminase comprises the mutations N46C, S73Y, and A162V (TadA-CD-28, FIG. 51E, PANCE, SEQ ID NO: 373) relative to the amino acid sequence of SEQ ID NO: 39. In some embodiments, the evolved TadA-Dual deaminase comprises the mutations N46V, Q71H, and S73P (TadA-CD-29, FIG. 51E, PANCE, SEQ ID NO: 374) relative to the amino acid sequence of SEQ ID NO: 39. In some embodiments, the evolved TadA-Dual deaminase comprises the mutations N46C and S73P (TadA-CD-30, FIG. 51E, PANCE, SEQ ID NO: 375) relative to the amino acid sequence of SEQ ID NO: 39. In some embodiments, the evolved TadA-Dual deaminase comprises the mutations N46C, S73P, and A162V (TadA-CD-31, FIG. 51E, PANCE, SEQ ID NO: 376) relative to the amino acid sequence of SEQ ID NO: 39. In some embodiments, the evolved TadA-Dual deaminase comprises the mutations N46V and S73P (TadA-CD-32, FIG. 51E, PANCE, SEQ ID NO: 377) relative to the amino acid sequence of SEQ ID NO: 39. In some embodiments, the evolved TadA-Dual deaminase comprises the mutation N46V (TadA-CD-33, FIG. 51E, PANCE, SEQ ID NO: 378) relative to the amino acid sequence of SEQ ID NO: 39. In some embodiments, the evolved TadA-Dual deaminase comprises the mutations N46V and R48P (TadA-CD-34, FIG. 51E, PANCE, SEQ ID NO: 379) relative to the amino acid sequence of SEQ ID NO: 39. In some embodiments, the evolved TadA-Dual deaminase comprises the mutations N46CV and S73P (TadA-CD-35, FIG. 51E, PANCE, SEQ ID NO: 380) relative to the amino acid sequence of SEQ ID NO: 39. In some embodiments, the evolved TadA-Dual deaminase comprises the mutations L34M, N46L and S73P (TadA-CD-36, FIG. 51E, PANCE, SEQ ID NO: 381) relative to the amino acid sequence of SEQ ID NO: 39. In some embodiments, the evolved TadA-Dual deaminase comprises the mutations N46L and S73P (TadA-CD-37, FIG. 51E, PANCE, SEQ ID NO: 382) relative to the amino acid sequence of SEQ ID NO: 39. In some embodiments, the evolved TadA-Dual deaminase comprises the mutations N46L, r48P, R64K and S73P (TadA-CD-38, FIG. 51E, PANCE, SEQ ID NO: 383) relative to the amino acid sequence of SEQ ID NO: 39.

In some embodiments, TadA-CD deaminases evolved from the TadA-Dual deaminase have improved specificity toward cytosine bases. In some embodiments, evolved TadA-CD deaminases exhibit similar cytosine on-target activity as other evolved deaminases described herein. In some embodiments, evolved deaminases evolved from the TadA-Dual deaminase have increased specificity toward cytosine bases and decreased specificity toward adenosine bases. In some embodiments, deaminases evolved from the TadA-Dual deaminases exhibit no residual A-to-G base editing (e.g., TadA-CD-1 through TadA-CD-38).

In some embodiments, TadA-CD-1 exhibits no residual A-to-G base editing when incorporated into the BE4max architecture. In some embodiments, TadA-CD-2 exhibits no residual A-to-G base editing when incorporated into the BE4max architecture. In some embodiments, TadA-CD-3 exhibits no residual A-to-G base editing when incorporated into the BE4max architecture. In some embodiments, TadA-CD-4 exhibits no residual A-to-G base editing when incorporated into the BE4max architecture. In some embodiments, TadA-CD-5 exhibits no residual A-to-G base editing when incorporated into the BE4max architecture. The above description is not intended to be limiting in any way, and the evolved TadA-CD deaminases, as described herein, may be used with any suitable architecture known to one of skill in the art.

In some embodiments, the TadA-CDs evolved from TadA-dual comprise at least 80%, 85%, 90%, 95%, 98%, 99%, or 99.5% identical to any of the amino acid sequences listed in Table 11.

In some embodiments, any one of the deaminases listed in Table 11 may further comprise a V106W mutation. In some embodiments, the TadA-CD variants comprise at least 80%, at least 85%, at least 90%, at least 95%, at least 98%, at least 99%, or at least 99.5% to any of the amino acid sequences listed in Table 10, wherein anyone of the sequences listed in Table 11 further comprise a V106W mutation.

TABLE 11
List of exemplary mutated TadA-CDs relative derived from TadA-Dual
(SEQ ID NO: 39). Sequences of TadA-8e and TadA-dual are provided as a reference.

| | | |
|---|---|---|
| TadA-8e | MSEVEFSHEYWMRHALTLAKRARDEREVPVGAVLVLNNRVIGEGWNRA | 41 |
| (substrate) | IGLHDPTAHAEIMALRQGGLVMQNYRLIDATLYVTFEPCVMCAGAMIHS | |
| | RIGRVVFGVRNSKRGAAGSLMNVLNYPGMNHRVEITEGILADECAALLCD | |
| | FYRMPRQVFNAQKKAQSSIN | |
| TadA-dual | MSEVEFSHEYWMRHALTLAKRARDEGEAPVGAVLVLNNRVIGEGWNRR | 39 |
| | IGLHDPTAHAEIMALRQGGLVMQNSRLIDATLYVTFEPCVMCAGAMINS | |
| | RIGRVVFGVRNSKRGAAGSLMNVLNYPGMNHRVEITEGILADECAALLCD | |
| | FYRMPRHVFNAQKKAQSSIN | |
| TadA-CD-1 | MSEVEFSHEYWMRHALTLAKRARDEGEAPVGAVLVLNNRVIGEGWIRRI | 42 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-2 | MSEVEFSHEYWMRHALTLAKRARDEGEAPVGAVLVLNNRVIGEGWTRRI | 43 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRHVFNAQKKAQSSIN | |
| TadA-CD-3 | MSEVEFSHEYWMRHALTLAKRARDEGEAPVGAVLVLNNRVIGEGWTRRI | 44 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-4 | MSEVEFSHEYWMRHALTLAKRARDEGEAPVGAVLVLNNRVIGEGWVRRI | 45 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-5 | MSEVEFSHEYWMRHALTLAKRARDEGEAPVGAVLVLNNRVIGEGWVRRI | 46 |
| | GLHDPTAHAEIMALRQGGLVMQNPRLIDATLYVTFEPCVMCAGAMINSR | |
| | IGRVVFSVRNSKRGAAGSLMNVLNYPGMNHRVEITEGILADECAALLCDF | |
| | YRMPRQVFNAQKKAQSSIN | |
| TadA-CD-6 | MSEVEFSHEYWMRHALTLAKRARDEGEAPVGAVLVLNNRVIGEGWLRRI | 47 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-7 | MSEVEFSHEYWMRHALTLAKRARDEREAPVGAVLVLNNRVIGEGWLRPI | 48 |
| | GLHDPTAHAEIMALRQGGLVMQNPRLIDATLYVTFEPCVMCAGAMIHSR | |
| | IGRVVFGVRNSKRGAAGSLMNVLNYPGMNHRVEITEGILADECAALLCDF | |
| | YRMPRQVFNAQKKAQSSIN | |
| TadA-CD-8 | MSEVEFSHEYWMRHALTLAKRARDEGEAPVGAVLVLNNRVIGEGWCRRI | 49 |
| | GLHDPTAHAEIMALRQGGLVMQNSRLIDATLYVTFEPCVMCAGAMIHSR | |
| | IGRVVFGVRNSKRGAAGSLMNVLNYPGMNHRVEITEGILADECAALLCDF | |
| | YRMPRQVFNAQKKAQSSIN | |
| TadA-CD-9 | MSEVEFSHEYWMRHALTLAKRARDEGEAPVGAVLVLNNRVIGEGWVRRI | 50 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-10 | MSEVEFSHEYWMRHALTLAKRARDEGEAPVGAVLVLNNRVIGEGWVRRI | 51 |
| | GLHDPTAHAEIMALRQGGLVMHNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-11 | MSEVEFSHEYWMRHALTLAKRARDEGEAPVGAVLVLNNRVIGEGWLRRI | 52 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-12 | MSEVEFSHEYWMRHALTLAKRARDEGEAPVGAVLVLNNRVIGEGWCRRI | 53 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-13 | MSEVEFSHEYWMRHALTLAKRARDEGEAPVGAVLVLNNRVIGEGWCRRI | 54 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKVQSSIN | |
| TadA-CD-14 | MSEVEFSHEYWMRHALTLAKRARDEGEAPVGAVLVLNNRVIGEGWIRRI | 359 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-15 | MSEVEFSHEYWMRHALTLAKRARDEGEAPVGAVLVLNNRVIGEGWNRR | 360 |
| | IGLHDPTAHAEIMALRQGGLVMSNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-16 | MSEVEFSHEYWMRHALTLAKRARDEGEAPVGAVLVLNNRVIGEGWLRRI | 361 |
| | GLHDPTAHAEIMALRQGGLVMQNPRLIDATLYVTFEPCVMCAGAMIHSR | |
| | IGRVVFGVRNSKRGAAGSLMNVLNYPGMNHRVEITEGILADECAALLCDF | |
| | YRMPRQVFNAQKKAQSSIN | |
| TadA-CD-17 | MSEVEFSHEYWMRHALTLAKRARDEGEAPVGAVLVLNNRVIGEGWLRRI | 362 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-18 | MSEVEFSHEYWMRHALTLAKRARDEGEAPVGAVLVLNNRVIGEGWNRA | 363 |
| | IGLHDPTAHAEIMALRQGGLVMQNPRLIDATLYVTFEPCVMCAGAMINS | |
| | RIGRVVFGVRNSKRGAAGSLMNVLNYPGMNHRVEITEGILADECAALLCD | |
| | FYRMPRQVFNAQKKAQSSIN | |
| TadA-CD-19 | MSEVEFSHEYWMRHALTLAKRARDEGEAPVGAVLVLNNRVIGEGWVRRI | 364 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-20 | MSEVEFSHEYWMRHALTLAKRARDEGEAPVGAVLVLNNRVIGEGWVRRI | 365 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-21 | MSEVEFSHEYWMRHALTLAKRARDEGEVPVGAVLVLNNRVIGEGWLRAI | 366 |
| | GLHDPTAHAEIMALRQGGLVMQNYRLIDATLYVTFEPCVMCAGAMIHSR | |
| | IGRVVFGVRNSKRGAAGSLMNVLNYPGMNHRVEITEGILADECAALLCDF | |
| | YRMPRQVFNAQKKAQSSIN | |
| TadA-CD-22 | MSEVEFSHEYWMRHALTLAKRARDEGEAPVGAVLVLNNRVIGEGWIRRI | 367 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-23 | MSEVEFSHEYWMRHALTLAKRARDEGEAPVGAVLVLNNRVIGEGWVRRI | 368 |
| | GLHDPTAHAEIMALRQGGLVMQNPRLIDATLYVTFEPCVMCAGAMINSR | |
| | IGRVVFSVRNSKRGAAGSLMNVLNYPGMNHRVEITEGILADECAALLCDF | |
| | YRMPRQVFNAQKKAQSSIN | |
| TadA-CD-24 | MSEVEFSHEYWMRHALTLAKRARDEGEAPVGAVLVLNNRVIGEGWNRP | 369 |
| | IGLHDPTAHAEIMALRQGGLVMQNHRLIDAPLYVTFEPCVMCAGAMINS | |
| | RIGRVVFGVRNSKRGAAGSLMNVLNYPGMNHRVEITEGILADECAALLCD | |
| | FYRMPRQVFNAQKKAQSSIN | |
| TadA-CD-25 | MSEVEFSHEYWMRHALTLAKRARDEGEVPVGAVLVLNNRVIGEGWIRAI | 370 |
| | GLHDPTAHAEIMALRQGGLVMQNYRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-26 | MSEVEFSHEYWMRHALTLAKRARDEGEAPVGAVLVLNNRVIGEGWVRRI | 371 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-27 | MSEVEFSHEYWMRHALTLAKRARDEGEAPVGAVLVLNNRVIGEGWLRRI | 372 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-28 | MSEVEFSHEYWMRHALTLAKRARDEGEAPVGAVLVLNNRVIGEGWCRRI | 373 |
| | GLHDPTAHAEIMALRQGGLVMQNYRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKVQSSIN | |
| TadA-CD-29 | MSEVEFSHEYWMRHALTLAKRARDEGEAPVGAVLVLNNRVIGEGWVRRI | 374 |
| | GLHDPTAHAEIMALRQGGLVMHNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-30 | MSEVEFSHEYWMRHALTLAKRARDEGEAPVGAVLVLNNRVIGEGWCRRI | 375 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-31 | MSEVEFSHEYWMRHALTLAKRARDEGEAPVGAVLVLNNRVIGEGWCRRI | 376 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKVQSSIN | |
| TadA-CD-32 | MSEVEFSHEYWMRHALTLAKRARDEGEAPVGAVLVLNNRVIGEGWVRRI | 377 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-33 | MSEVEFSHEYWMRHALTLAKRARDEGEAPVGAVLVLNNRVIGEGWVRRI | 378 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-34 | MSEVEFSHEYWMRHALTLAKRARDEGEAPVGAVLVLNNRVIGEGWVRPI | 379 |
| | GLHDPTAHAEIMALRQGGLVMQNSRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-35 | MSEVEFSHEYWMRHALTLAKRARDEGEAPVGAVLVLNNRVIGEGWCRRI | 380 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-36 | MSEVEFSHEYWMRHALTLAKRARDEGEAPVGAVMVLNNRVIGEGWLR | 381 |
| | RIGLHDPTAHAEIMALRQGGLVMQNPRLIDATLYVTFEPCVMCAGAMIN | |
| | SRIGRVVFGVRNSKRGAAGSLMNVLNYPGMNHRVEITEGILADECAALLC | |
| | DFYRMPRQVFNAQKKAQSSIN | |
| TadA-CD-37 | MSEVEFSHEYWMRHALTLAKRARDEGEAPVGAVLVLNNRVIGEGWLRRI | 382 |
| | GLHDPTAHAEIMALRQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |
| TadA-CD-38 | MSEVEFSHEYWMRHALTLAKRARDEGEAPVGAVLVLNNRVIGEGWLRPI | 383 |
| | GLHDPTAHAEIMALKQGGLVMQNPRLIDATLYVTFEPCVMCAGAMINSR | |
| | IGRVVFGVRNSKRGAAGSLMNVLNYPGMNHRVEITEGILADECAALLCDF | |
| | YRMPRQVFNAQKKAQSSIN | |

napDNAbp Domains

The base editors described herein comprise a nucleic acid programmable DNA binding (napDNAbp) domain. The napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp domain to localize and bind to a complementary sequence of the target strand. Binding of the napDNAbp domain to a complementary sequence enables the nucleobase modification domain (i.e., the adenosine deaminase domain) of the base editor to access and enzymatically deaminate a target base in the target strand.

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), the entire contents of which is hereby incorporated by reference.

The below description of various napDNAbps which can be used in connection with the disclosed adenosine deaminases is not meant to be limiting in any way. The base editors may comprise the canonical SpCas9, or any ortholog Cas9 protein, or any variant Cas9 protein—including any naturally-occurring variant, mutant, or otherwise engineered version of Cas9—that is known or which can be made or evolved through a directed evolutionary or otherwise mutagenic process. In various embodiments, the napDNAbp has a nickase activity, i.e., only cleave one strand of the target DNA sequence. In other embodiments, the napDNAbp has an inactive nuclease, e.g., are “dead” proteins. Other variant Cas9 proteins that may be used are those having a smaller molecular weight than the canonical SpCas9 (e.g., for easier delivery) or having modified or rearranged primary amino acid sequence (e.g., the circular permutant forms). The base editors described herein may also comprise Cas9 equivalents, including Cas12a/Cpf1 proteins. The napDNAbps used herein (e.g., SpCas9, SaCas9, or SaCas9 variant or SpCas9 variant) may also may also contain various modifications that alter/enhance their PAM specifities. The disclosure contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to any of the Cas9 proteins disclosed herein. In some embodiments, the napDNAbp domain comprises a nickase variant of a wild-type Cas9. In some embodiments, the napDNAbp domain comprises any of the Cas9 nickases disclosed herein.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, and H588A and D16A in reference to the Nme2Cas9 sequence, and to equivalent amino acid positions in other Cas9 variants or Cas9 equivalents.

As used herein, the term “Cas protein” refers to a full-length Cas protein obtained from nature, a recombinant Cas protein having a sequences that differs from a naturally-occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas9 proteins, as well as Cas9 equivalents, variants (e.g., Cas9 nickase (nCas9) or nuclease inactive Cas9 (dCas9)) homologs, orthologs, or paralogs, whether naturally-occurring or non-naturally-occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The term “Cas9” or “Cas9 domain” embraces any naturally-occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular napDNAbp that is employed in the base editors of the disclosure.

As used herein, the terms “compact Cas9 protein”, “compact napDNAbp” and “compact variant [of a Cas protein]” refers to a Cas9 protein or variant that has an amino acid length of less than about 1250 amino acids. In some embodiments, a compact Cas9 protein or compact napDNAbp contains less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids in length. These terms also embrace any Cas9 protein or variant encoded by a nucleic acid sequence having a length of less than about 3750 nucleotides. The base editors of the disclosure may comprise compact napDNAbps and/or compact Cas9 proteins. In some embodiments, the compact Cas9 protein is about 350 amino acids shorter than a SpCas9. In some embodiments, the compact Cas9 protein is about 1000 amino acids in length. In some embodiments, the compact protein is a compact variant of S. pyogenes Cas9 (SpCas9), Cpf1, CasX, CasY, C2c1, C2c2, C2c3, Cas12a, Cas12b, Cas12g, Cas12h, Cas12i, Cas13b, Cas13c, Cas13d, Cas14, Csn2, Cas3, or CasΦ. A “compact variant” may refer to a Cas9 protein that has one or more truncations, or one or more deletions, relative to a wild-type Cas9 protein, such as a wild-type SpCas9 or Cpf1.

Additional Cas9 sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Nat. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference), and also provided below.

Examples of Cas9 and Cas9 equivalents are provided as follows; however, these specific examples are not meant to be limiting. The base editors of the present disclosure may use any suitable napDNAbp, including any suitable Cas9 or Cas9 equivalent.

In some embodiments, the Cas9 comprises or is derived from a wild-type SaCas9 (e.g., Staphylococcus aureus, 1053AA, 123 kDa). In some embodiments, the wild type SaCas9 comprises the following amino acid sequence:

(SEQ ID NO: 347)
MGKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKLSEEEFSA
ALLHLAKRRGVHNVNEVEEDTGNELSTKEQISRNSKALEEKYVAELQLERLKKDGE
VRGSINRFKTSDYVKEAKQLLKVQKAYHQLDQSFIDTYIDLLETRRTYYEGPGEGSP
FGWKDIKEWYEMLMGHCTYFPEELRSVKYAYNADLYNALNDLNNLVITRDENEKL
EYYEKFQIIENVFKQKKKPTLKQIAKEILVNEEDIKGYRVTSTGKPEFTNLKVYHDIK
DITARKEIIENAELLDQIAKILTIYQSSEDIQEELTNLNSELTQEEIEQISNLKGYTGTH
NLSLKAINLILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQTNERIEEIIRT
TGKENAKYLIEKIKLHDMQEGKCLYSLEAIPLEDLLNNPFNYEVDHIIPRSVSFDNSF
NNKVLVKQEENSKKGNRTPFQYLSSSDSKISYETFKKHILNLAKGKGRISKTKKEYL
LEERDINRFSVQKDFINRNLVDTRYATRGLMNLLRSYFRVNNLDVKVKSINGGFTSF
LRRKWKFKKERNKGYKHHAEDALIIANADFIFKEWKKLDKAKKVMENQMFEEKQ
AESMPEIETEQEYKEIFITPHQIKHIKDFKDYKYSHRVDKKPNRKLINDTLYSTRKDD
KGNTLIVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNSRNKVV
KLSLKPYRFDVYLDNGVYKFVTVKNLDVIKKENYYEVNSKCYEEAKKLKKISNQA
EFIASFYKNDLIKINGELYRVIGVNNDLLNRIEVNMIDITYREYLENMNDKRPPHIIKT
IASKTQSIKKYSTDILGNLYEVKSKKHPQIIKK

In some embodiments, the sequence of SaCas9 comprises at least at least 80%, at least 85%, at least 90%, at least 95%, at least 99%, at least 99.5%, or at least 99.8% sequence identity to SEQ ID NO: 347.

In some embodiments, the Cas9 comprises or is derived from a wild-type SpCas9 (e.g., SpCas9, Streptococcus pyogenes M1, SwissProt Accession No. Q99ZW2, Wild type). In some embodiments, the wild type SaCas9 comprises the following amino acid sequence:

(SEQ ID NO: 200)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRL
ENLIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQL
KEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTI
LDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYE
TRIDLSQLGGD

In some embodiments, the sequence of SpCas9 comprises at least at least 80%, at least 85%, at least 90%, at least 95%, at least 99%, at least 99.5%, or at least 99.8% sequence identity to SEQ ID NO: 200.

### Cas Nickases

In some embodiments, the disclosed base editors may comprise a napDNAbp domain that comprises a Cas nickase. In some embodiments, the base editors described herein comprise a Cas9 nickase. In some embodiments, any of the disclosed base editors or vectors may comprise an S. pyogenes Cas9 nickase (SpCas9n, or nCas9) containing a D10A mutation. In some embodiments, any of the disclosed base editors may comprise an Nme2Cas9 nickase (Nme2Cas9n) or an eNme2-C Cas9 nickase (eNme2-C Cas9n), each of which contains a D16A mutation.

The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In some embodiments, the napDNAbp domain of any of the disclosed base editors comprises an S. pyogenes Cas9 nickase (SpCas9n). In some embodiments, the napDNAbp domain of any of the disclosed based editors is comprises at least 80%, at least 85%, at least 90%, at least 95%, or at least 99% sequence identity to SEQ ID NO: 343. In some embodiments, the napDNAbp domain of any of the disclosed base editors comprises the amino acid sequence of SEQ ID NO: 343.

In some embodiments, the napDNAbp domain of any of the disclosed base editors comprises an S. aureus Cas9 nickase (SaCas9n). In some embodiments, the napDNAbp domain of any of the disclosed based editors is comprises at least 80%, at least 85%, at least 90%, at least 95%, or at least 99% sequence identity to SEQ ID NO: 351. In some embodiments, the napDNAbp domain of any of the disclosed base editors comprises the amino acid sequence of SEQ ID NO: 351.

In some embodiments, the napDNAbp domain of any of the disclosed base editors comprises an N. meningitidis Cas9 nickase (Nme2Ca9n), or a variant thereof. In some embodiments, the napDNAbp domain of any of the disclosed based editors is comprises at least 80%, at least 85%, at least 90%, at least 95%, or at least 99% sequence identity to SEQ ID NO: 352 or 353. In some embodiments, the napDNAbp domain of any of the disclosed base editors comprises the amino acid sequence of SEQ ID NO: 352 or 353. In some embodiments, the napDNAbp domain comprises the amino acid sequence of SEQ ID NO: 353. The eNme2-C Cas9 (SEQ ID NO: 353) variant shows a preference for targeting NNNNCN (N₄CN) PAMs. Base editors containing this eNme2-C variant have generated efficiencies of base editing of about 60% or higher on N₄CC PAMs in human cells, which represents a two-fold improvement relative to base editors containing wild-type Nme2Cas9.

In some embodiments, the napDNAbp domain of any of the disclosed base editors comprises a wild-type Nme2Cas9 nuclease (SEQ ID NO: 349).

In various embodiments, the Cas nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLAIGTNSVGWAVITDEYKVPSKKFKVLGNTD | SEQ ID NO: |
| Streptococcus | RHSIKKNLIGALLFDSGETAEATRLKRTARRRYTRRKNRIC | 343 |
| pyogenes | YLQEIFSNEMAKVDDSFFHRLEESFLVEEDKKHERHPIFG | |
| Q99ZW2 | NIVDEVAYHEKYPTIYHLRKKLVDSTDKADLRLIYLALAH | |
| Cas9 with | MIKFRGHFLIEGDLNPDNSDVDKLFIQLVQTYNQLFEENPI | |
| D10A | NASGVDAKAILSARLSKSRRLENLIAQLPGEKKNGLFGNL | |
| | IALSLGLTPNFKSNFDLAEDAKLQLSKDTYDDDLDNLLA | |
| | QIGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVY | |
| | NELTKVKYVTEGMRKPAFLSGEQKKAIVDLLFKTNRKVT | |
| | VKQLKEDYFKKIECFDSVEISGVEDRFNASLGTYHDLLKII | |
| | KDKDFLDNEENEDILEDIVLTLTLFEDREMIEERLKTYAHL | |
| | FDDKVMKQLKRRRYTGWGRLSRKLINGIRDKQSGKTILD | |
| | FLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDV | |
| | DHIVPQSFLKDDSIDNKVLTRSDKNRGKSDNVPSEEVVKK | |
| | MKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTA | |
| | LIKKYPKLESEFVYGDYKVYDVRKMIAKSEQEIGKATAK | |
| | YFFYSNIMNFFKTEITLANGEIRKRPLIETNGETGEIVWDK | |
| | GRDFATVRKVLSMPQVNIVKKTEVQTGGFSKESILPKRNS | |
| | DKLIARKKDWDPKKYGGFDSPTVAYSVLVVAKVEKGKS | |
| | KKLKSVKELLGITIMERSSFEKNPIDFLEAKGYKEVKKDLI | |
| | IKLPKYSLFELENGRKRMLASAGELQKGNELALPSKYVN | |
| | FLYLASHYEKLKGSPEDNEQKQLFVEQHKHYLDEIIEQISE | |
| | FSKRVILADANLDKVLSAYNKHRDKPIREQAENIIHLFTLT | |
| | NLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTD | SEQ ID NO: |
| Streptococcus | RHSIKKNLIGALLFDSGETAEATRLKRTARRRYTRRKNRIC | 344 |
| pyogenes Q99ZW2 | YLQEIFSNEMAKVDDSFFHRLEESFLVEEDKKHERHPIFG | |
| Cas9 with | NIVDEVAYHEKYPTIYHLRKKLVDSTDKADLRLIYLALAH | |
| E762A | MIKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLA | |
| | QIGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVY | |
| | NELTKVKYVTEGMRKPAFLSGEQKKAIVDLLFKTNRKVT | |
| | VKQLKEDYFKKIECFDSVEISGVEDRFNASLGTYHDLLKII | |
| | KDKDFLDNEENEDILEDIVLTLTLFEDREMIEERLKTYAHL | |
| | FDDKVMKQLKRRRYTGWGRLSRKLINGIRDKQSGKTILD | |
| | FLKSDGFANRNFMQLIHDDSLTFKEDIQKAQVSGQGDSL | |
| | HEHIANLAGSPAIKKGILQTVKVVDELVKVMGRHKPENIV | |
| | IAMARENQTTQKGQKNSRERMKRIEEGIKELGSQILKEHP | |
| | VENTQLQNEKLYLYYLQNGRDMYVDQELDINRLSDYDV | |
| | DHIVPQSFLKDDSIDNKVLTRSDKNRGKSDNVPSEEVVKK | |
| | MKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTA | |
| | LIKKYPKLESEFVYGDYKVYDVRKMIAKSEQEIGKATAK | |
| | YFFYSNIMNFFKTEITLANGEIRKRPLIETNGETGEIVWDK | |
| | GRDFATVRKVLSMPQVNIVKKTEVQTGGFSKESILPKRNS | |
| | DKLIARKKDWDPKKYGGFDSPTVAYSVLVVAKVEKGKS | |
| | KKLKSVKELLGITIMERSSFEKNPIDFLEAKGYKEVKKDLI | |
| | IKLPKYSLFELENGRKRMLASAGELQKGNELALPSKYVN | |
| | FLYLASHYEKLKGSPEDNEQKQLFVEQHKHYLDEIIEQISE | |
| | FSKRVILADANLDKVLSAYNKHRDKPIREQAENIIHLFTLT | |
| | NLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTD | SEQ ID NO: |
| Streptococcus | RHSIKKNLIGALLFDSGETAEATRLKRTARRRYTRRKNRIC | 345 |
| pyogenes Q99ZW2 | NIVDEVAYHEKYPTIYHLRKKLVDSTDKADLRLIYLALAH | |
| Cas9 with | YLQEIFSNEMAKVDDSFFHRLEESFLVEEDKKHERHPIFG | |
| H983A | MIKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLA | |
| | QIGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVY | |
| | NELTKVKYVTEGMRKPAFLSGEQKKAIVDLLFKTNRKVT | |
| | VKQLKEDYFKKIECFDSVEISGVEDRFNASLGTYHDLLKII | |
| | KDKDFLDNEENEDILEDIVLTLTLFEDREMIEERLKTYAHL | |
| | FDDKVMKQLKRRRYTGWGRLSRKLINGIRDKQSGKTILD | |
| | FLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDV | |
| | DHIVPQSFLKDDSIDNKVLTRSDKNRGKSDNVPSEEVVKK | |
| | MKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHAAHDAYLNAVVGTA | |
| | LIKKYPKLESEFVYGDYKVYDVRKMIAKSEQEIGKATAK | |
| | YFFYSNIMNFFKTEITLANGEIRKRPLIETNGETGEIVWDK | |
| | GRDFATVRKVLSMPQVNIVKKTEVQTGGFSKESILPKRNS | |
| | DKLIARKKDWDPKKYGGFDSPTVAYSVLVVAKVEKGKS | |
| | KKLKSVKELLGITIMERSSFEKNPIDFLEAKGYKEVKKDLI | |
| | IKLPKYSLFELENGRKRMLASAGELQKGNELALPSKYVN | |
| | FLYLASHYEKLKGSPEDNEQKQLFVEQHKHYLDEIIEQISE | |
| | FSKRVILADANLDKVLSAYNKHRDKPIREQAENIIHLFTLT | |
| | NLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTD | SEQ ID NO: |
| Streptococcus | RHSIKKNLIGALLFDSGETAEATRLKRTARRRYTRRKNRIC | 346 |
| pyogenes | YLQEIFSNEMAKVDDSFFHRLEESFLVEEDKKHERHPIFG | |
| Q99ZW2 | NIVDEVAYHEKYPTIYHLRKKLVDSTDKADLRLIYLALAH | |
| Cas9 with | MIKFRGHFLIEGDLNPDNSDVDKLFIQLVQTYNQLFEENPI | |
| D986A | NASGVDAKAILSARLSKSRRLENLIAQLPGEKKNGLFGNL | |
| | IALSLGLTPNFKSNFDLAEDAKLQLSKDTYDDDLDNLLA | |
| | QIGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVY | |
| | NELTKVKYVTEGMRKPAFLSGEQKKAIVDLLFKTNRKVT | |
| | VKQLKEDYFKKIECFDSVEISGVEDRFNASLGTYHDLLKII | |
| | KDKDFLDNEENEDILEDIVLTLTLFEDREMIEERLKTYAHL | |
| | FDDKVMKQLKRRRYTGWGRLSRKLINGIRDKQSGKTILD | |
| | FLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDV | |
| | DHIVPQSFLKDDSIDNKVLTRSDKNRGKSDNVPSEEVVKK | |
| | MKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHAAYLNAVVGTA | |
| | LIKKYPKLESEFVYGDYKVYDVRKMIAKSEQEIGKATAK | |
| | YFFYSNIMNFFKTEITLANGEIRKRPLIETNGETGEIVWDK | |
| | GRDFATVRKVLSMPQVNIVKKTEVQTGGFSKESILPKRNS | |
| | DKLIARKKDWDPKKYGGFDSPTVAYSVLVVAKVEKGKS | |
| | KKLKSVKELLGITIMERSSFEKNPIDFLEAKGYKEVKKDLI | |
| | IKLPKYSLFELENGRKRMLASAGELQKGNELALPSKYVN | |
| | FLYLASHYEKLKGSPEDNEQKQLFVEQHKHYLDEIIEQISE | |
| | FSKRVILADANLDKVLSAYNKHRDKPIREQAENIIHLFTLT | |
| | NLGAPAAFKYFDTTIDRKRYTSTKEVLDATLIHQSITGLYE | |
| | TRIDLSQLGGD | |
| Cas9 nickase | MGKRNYILGLAIGITSVGYGIIDYETRDVIDAGVRLFKEA | SEQ ID NO: |
| Staphylococc | NVENNEGRRSKRGARRLKRRRRHRIQRVKKLLFDYNLLT | 351 |
| us aureus | DHSELSGINPYEARVKGLSQKLSEEEFSAALLHLAKRRGV | |
| (SaCas9) | HNVNEVEEDTGNELSTKEQISRNSKALEEKYVAELQLERL | |
| with D10A | KKDGEVRGSINRFKTSDYVKEAKQLLKVQKAYHQLDQS | |
| | FIDTYIDLLETRRTYYEGPGEGSPFGWKDIKEWYEMLMG | |
| | HCTYFPEELRSVKYAYNADLYNALNDLNNLVITRDENEK | |
| | LEYYEKFQIIENVFKQKKKPTLKQIAKEILVNEEDIKGYRV | |
| | TSTGKPEFTNLKVYHDIKDITARKEIIENAELLDQIAKILTI | |
| | YQSSEDIQEELTNLNSELTQEEIEQISNLKGYTGTHNLSLK | |
| | AINLILDELWHTNDNQIAIFNRLKLVPKKVDLSQQKEIPTT | |
| | LVDDFILSPVVKRSFIQSIKVINAIIKKYGLPNDIIIELAREK | |
| | NSKDAQKMINEMQKRNRQTNERIEEIIRTTGKENAKYLIE | |
| | KIKLHDMQEGKCLYSLEAIPLEDLLNNPFNYEVDHIIPRSV | |
| | SFDNSFNNKVLVKQEENSKKGNRTPFQYLSSSDSKISYET | |
| | FKKHILNLAKGKGRISKTKKEYLLEERDINRFSVQKDFIN | |
| | RNLVDTRYATRGLMNLLRSYFRVNNLDVKVKSINGGFTS | |
| | FLRRKWKFKKERNKGYKHHAEDALIIANADFIFKEWKKL | |
| | DKAKKVMENQMFEEKQAESMPEIETEQEYKEIFITPHQIK | |
| | HIKDFKDYKYSHRVDKKPNRKLINDTLYSTRKDDKGNTL | |
| | IVNNLNGLYDKDNDKLKKLINKSPEKLLMYHHDPQTYQ | |
| | KLKLIMEQYGDEKNPLYKYYEETGNYLTKYSKKDNGPVI | |
| | KKIKYYGNKLNAHLDITDDYPNSRNKVVKLSLKPYRFDV | |
| | YLDNGVYKFVTVKNLDVIKKENYYEVNSKCYEEAKKLK | |
| | KISNQAEFIASFYKNDLIKINGELYRVIGVNNDLLNRIEVN | |
| | MIDITYREYLENMNDKRPPHIIKTIASKTQSIKKYSTDILG | |
| | NLYEVKSKKHPQIIKK | |
| Cas9 nickase | MAAFKPNPINYILGLAIGIASVGWAMVEIDEEENPIRLIDL | SEQ ID NO: |
| Neisseria | GVRVFERAEVPKTGDSLAMARRLARSVRRLTRRRAHRLL | 352 |
| meningitidis | RARRLLKREGVLQAADFDENGLIKSLPNTPWQLRAAALD | |
| (Nme2Cas9) | RKLTPLEWSAVLLHLIKHRGYLSQRKNEGETADKELGAL | |
| with D16A | LKGVANNAHALQTGDFRTPAELALNKFEKESGHIRNQRG | |
| | DYSHTFSRKDLQAELILLFEKQKEFGNPHVSGGLKEGIET | |
| | LLMTQRPALSGDAVQKMLGHCTFEPAEPKAAKNTYTAER | |
| | FIWLTKLNNLRILEQGSERPLTDTERATLMDEPYRKSKLTY | |
| | AQARKLLGLEDTAFFKGLRYGKDNAEASTLMEMKAYHA | |
| | ISRALEKEGLKDKKSPLNLSSELQDEIGTAFSLFKTDEDIT | |
| | GRLKDRVQPEILEALLKHISFDKFVQISLKALRRIVPLMEQ | |
| | GKRYDEACAEIYGDHYGKKNTEEKIYLPPIPADEIRNPVV | |
| | LRALSQARKVINGVVRRYGSPARIHIETAREVGKSFKDRK | |
| | EIEKRQEENRKDREKAAAKFREYFPNFVGEPKSKDILKLR | |
| | LYEQQHGKCLYSGKEINLVRLNEKGYVEIDHALPFSRTW | |
| | DDSFNNKVLVLGSENQNKGNQTPYEYFNGKDNSREWQE | |
| | FKARVETSRFPRSKKQRILLQKFDEDGFKECNLNDTRYVN | |
| | RFLCQFVADHILLTGKGKRRVFASNGQITNLLRGFWGLRK | |
| | VRAENDRHHALDAVVVACSTVAMQQKITRFVRYKEMNA | |
| | FDGKTIDKETGKVLHQKTHFPQPWEFFAQEVMIRVFGKP | |
| | DGKPEFEEADTPEKLRTLLAEKLSSRPEAVHEYVTPLFVS | |
| | RAPNRKMSGAHKDTLRSAKRFVKHNEKISVKRVWLTEIK | |
| | LADLENMVNYKNGREIELYEALKARLEAYGGNAKQAFD | |
| | PKDNPFYKKGGQLVKAVRVEKTQESGVLLNKKNAYTIAD | |
| | NGDMVRVDVFCKVDKKGKNQYFIVPIYAWQVAENILPDI | |
| | DCKGYRIDDSYTFCFSLHKYDLIAFQKDEKSKVEFAYYIN | |
| | CDSSNGRFYLAWHDKGSKEQQFRISTQNLVLIQKYQVNE | |
| | LGKEIRPCRLKKRPPVR | |

### Cas9 Equivalents

In some embodiments, the base editors described herein can include any Cas9 equivalent. As used herein, the term “Cas9 equivalent” is a broad term that encompasses any napDNAbp that serves the same function as Cas9 in the present base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The base editors described here embrace any Cas9 equivalent that would provide the same or similar function as Cas9 despite that the Cas9 equivalent may be based on a protein that arose through convergent evolution. For instance, if Cas9 refers to a type II enzyme of the CRISPR-Cas system, a Cas9 equivalent can refer to a type V or type VI enzyme of the CRISPR-Cas system.

For example, Cas12e (CasX) is a Cas9 equivalent that reportedly has the same function as Cas9 but which evolved through convergent evolution. Thus, the Cas12e (CasX) protein described in Liu et al., “CasX enzymes comprises a distinct family of RNA-guided genome editors,” Nature, 2019, Vol. 566: 218-223, is contemplated to be used with the base editors described herein. In addition, any variant or modification of Cas12e (CasX) is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to Cas12e (CasX) or Cas12d (CasY), which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-Cas12e and CRISPR-Cas12d, which are among the most compact systems yet discovered. In some embodiments, Cas9 refers to Cas12e, or a variant of Cas12e. In some embodiments, Cas9 refers to a Cas12d, or a variant of Cas12d. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12e (CasX) or Cas12d (CasY) protein. In some embodiments, the napDNAbp is a naturally-occurring Cas12e (CasX) or Cas12d (CasY) protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), C2C3Cas12e (CasX), Cas12d (CasY), Cas12a (Cpf1), Cas12b1 (C2c1), Cas13a (C2c2), Cas12c (C2c3), Argonaute. One example of a nucleic acid programmable DNA-binding protein that has different PAM specificity than Cas9 is Clustered Regularly Interspaced Short Palindromic Repeats from Prevotella and Francisella 1 (i.e., Cas12a (Cpf1)). Similar to Cas9, Cas12a (Cpf1) is also a Class 2 CRISPR effector, but it is a member of the type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

In still other embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 200).

In various other embodiments, the napDNAbp can be any of the following proteins: a Cas9, a C2c3Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a variant thereof.

Cas9 Variants with Modified PAM Specificities

The base editors of the present disclosure may also comprise Cas9 variants with modified PAM specificities. For example, the base editors described herein may utilize any naturally-occurring or engineered variant of SpCas9 having expanded and/or relaxed PAM specificities which are described in the literature, including in Nishimasu et al., “Engineered CRISPR-Cas9 nuclease with expanded targeting space,” Science, 2018, 361: 1259-1262; Chatterjee et al., “Robust Genome Editing of Single-Base PAM Targets with Engineered ScCas9 Variants,” BioRxiv, Apr. 26, 2019. Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

The above description of various napDNAbps which can be used in connection with the presently disclose base editors is not meant to be limiting in any way. The base editors may comprise the canonical SpCas9, or any ortholog Cas9 protein, or any variant Cas9 protein—including any naturally-occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1).

In some embodiments, the SpCas9(H840A) comprises a sequence that is at least 80%, at least 85%, at least 90%, at least 95%, at least 99%, or at least 99.5% identical to the amino acid sequence in SEQ ID NO: 480.

SpCas9(H840A)
(SEQ ID NO: 386)
DKKYSIGLDIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSITGLYETRIDL
SQLGGD

In a particular embodiment, the Cas9 variant having expanded PAM capabilities is SpCas9 (H840A) VRQR, having the following amino acid sequence (with the V, R, Q, R substitutions relative to the SpCas9 (H840A) of SEQ ID NO: 480 show in bold underline. In addition, the methionine residue in SpCas9 (H840) was removed for SpCas9 (H840A) VRQR) (“SpCas9-VRQR”). This SpCas9 variant possesses an altered PAM-specificity which recognizes a PAM of 5′-NGA-3′ instead of the canonical PAM of 5′-NGG-3′:

SpCas9-VRQR
(SEQ ID NO: 74)
DKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSIDNKVLT
RSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFVSPTV
AYSVLVVAKVEKGKSKKLKSVKELLGITIMERSSFEKNPIDFLEAKGYKEVKKDLII
KLPKYSLFELENGRKRMLASARELQKGNELALPSKYVNFLYLASHYEKLKGSPEDN
EQKQLFVEQHKHYLDEIIEQISEFSKRVILADANLDKVLSAYNKHRDKPIREQAENII
HLFTLTNLGAPAAFKYFDTTIDRKQYRSTKEVLDATLIHQSITGLYETRIDLSQLGGD.

In another particular embodiment, the Cas9 variant having expanded PAM capabilities is SpCas9 (H840A) VQR, having the following amino acid sequence (with the V, Q, R substitutions relative to the SpCas9 (H840A) of SEQ ID NO: 480 shown in bold underline. In addition, the methionine residue in SpCas9 (H840) was removed for SpCas9 (H840A) VRQR) (“SpCas9-VQR”). This SpCas9 variant possesses an altered PAM-specificity which recognizes a PAM of 5′-NGA-3′ instead of the canonical PAM of 5′-NGG-3′:

SpCas9-VQR
(SEQ ID NO: 75)
DKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSIDNKVLT
RSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFVSPTV
AYSVLVVAKVEKGKSKKLKSVKELLGITIMERSSFEKNPIDFLEAKGYKEVKKDLII
KLPKYSLFELENGRKRMLASAGELQKGNELALPSKYVNFLYLASHYEKLKGSPEDN
EQKOLFVEQHKHYLDEIIEQISEFSKRVILADANLDKVLSAYNKHRDKPIREQAENII
HLFTLTNLGAPAAFKYFDTTIDRKQYRSTKEVLDATLIHQSITGLYETRIDLSQLGGD.

In another particular embodiment, the Cas9 variant having expanded PAM capabilities is SpCas9 (H840A) VRER, having the following amino acid sequence (with the V, R, E, R substitutions relative to the SpCas9 (H840A) of SEQ ID NO: 480 are shown in bold underline. In addition, the methionine residue in SpCas9 (H840) was removed for SpCas9 (H840A) VRER) (“SpCas9-VRER”). This SpCas9 variant possesses an altered PAM-specificity which recognizes a PAM of 5′-NGCG-3′ instead of the canonical PAM of 5′-NGG-3′:

SpCas9-VRER
(SEQ ID NO: 76)
DKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSIDNKVLT
RSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFVSPTV
AYSVLVVAKVEKGKSKKLKSVKELLGITIMERSSFEKNPIDFLEAKGYKEVKKDLII
KLPKYSLFELENGRKRMLASARELQKGNELALPSKYVNFLYLASHYEKLKGSPEDN
EQKQLFVEQHKHYLDEIIEQISEFSKRVILADANLDKVLSAYNKHRDKPIREQAENII
HLFTLTNLGAPAAFKYFDTTIDRKEYRSTKEVLDATLIHQSITGLYETRIDLSQLGGD.

In another embodiment, the Cas9 variant having expanded PAM capabilities is SpCas9-NG, as reported in Nishimasu et al., “Engineered CRISPR-Cas9 nuclease with expanded targeting space,” Science, 2018, 361: 1259-1262, which is incorporated herein by reference. SpCas9-NG (VRVRFRR), having the following amino acid sequence substitutions: R1335V, L1111R, D1135V, G1218R, E1219F, A1322R, and T1337R relative to the canonical SpCas9 sequence (SEQ ID NO: 200). This SpCas9 has a relaxed PAM specificity, i.e., with activity on a PAM of NGH (wherein H=A, T, or C). See Nishimasu et al., “Engineered CRISPR-Cas9 nuclease with expanded targeting space,” Science, 2018, 361: 1259-1262, which is incorporated herein by reference.

SpCas9-NG
(SEQ ID NO: 77)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLY
EYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGR
DFATVRKVLSMPQVNIVKKTEVQTGGFSKESIRPKRNSDKLIARKKDWDPKKYGGF
VSPTVAYSVLVVAKVEKGKSKKLKSVKELLGITIMERSSFEKNPIDFLEAKGYKEVK
KDLIIKLPKYSLFELENGRKRMLASARFLQKGNELALPSKYVNFLYLASHYEKLKGS
PEDNEQKQLFVEQHKHYLDEIIEQISEFSKRVILADANLDKVLSAYNKHRDKPIREQ
AENIIHLFTLTNLGAPRAFKYFDTTIDRKVYRSTKEVLDATLIHQSITGLYETRIDLSQ
LGGD.

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. Variant Cas9s may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Compact Cas9 Variants with Modified PAM Specificities

In some embodiments, the napDNAbp comprises a compact Cas protein, such as a Cas9 derived from C. jejuni, S. auricularis, N. meningitidis, or S. aureus. In exemplary embodiments, the napDNAbp comprises a CjCas9 nickase, a SauriCas9 nickase, an Nme2Cas9 nickase, an SaCas9 nickase, or an SaKKH-Cas9 nickase. In some embodiments, the napDNAbp is not an Nme2Cas9 protein or nickase. In some embodiments, the napDNAbp is not a SaCas9 protein or nickase.

In some embodiments, the disclosed base editors comprise a napDNAbp domain comprising a Cas9 ortholog derived from Neisseria meningitidis (Nme, or Nme2). In some embodiments, the napDNAbp domain comprises Nme2Cas9. In other embodiments, the napDNAbp domain is a Nme2Cas9 domain. In some embodiments, the disclosed base editors comprise a Nme2Cas9 nickase. Nme2Cas9 recognizes a simple dinucleotide PAM, NNNNCC, or N₄CC (where N is any nucleotide), as described in Edraki et al., Molecular Cell 73, 714-726, incorporated herein by reference. In other embodiments, the napDNAbp domain comprises a Nme2Cas9 variant. The variants of Nme2Cas9 may recognize a wider array of PAMs. In some embodiments, Nme2Cas9 variants of the present disclosure recognize single-nucleotide-pyrimidine PAMs. In some embodiments, the Nme2Cas9 variants recognize PAMs of the sequence NYN, where Y is any pyrimidine (i.e., C, T, or U). In other embodiments, the Nme2Cas9 variants recognize PAMs of the sequence NNNNCN, or N₄CN. In some embodiments, the Nme2Cas9 variant is eNme2Cas9 nickase (SEQ ID NO: 439). In some embodiments, the Nme2Cas9 variant is eNme2-C Cas9 nickase (SEQ ID NO: 353).

The sequence of wild-type Nme2Cas9 is set forth as SEQ ID NO: 349. In some embodiments, the disclosed base editors comprise a napDNAbp domain that has a sequence that is at least 90%, at least 95%, at least 98%, or at least 99% identical to SEQ ID NO: 349. In some embodiments, the disclosed base editor comprises a napDNAbp comprising SEQ ID NO:5. This protein may be referred to herein as engineered Nme2Cas9, or eNme2Cas9. In various embodiments, any of the disclosed TadCBEs comprise a variant of Nme2Cas9 or Nme2Cas9.

Wild-type Nme2Cas9
(SEQ ID NO: 349)
MAAFKPNPINYILGLDIGIASVGWAMVEIDEEENPIRLIDLGVRVFERAEVPKTGDSLA
MARRLARSVRRLTRRRAHRLLRARRLLKREGVLQAADFDENGLIKSLPNTPWQLRA
AALDRKLTPLEWSAVLLHLIKHRGYLSQRKNEGETADKELGALLKGVANNAHALQT
GDFRTPAELALNKFEKESGHIRNQRGDYSHTFSRKDLQAELILLFEKQKEFGNPHVSG
GLKEGIETLLMTQRPALSGDAVQKMLGHCTFEPAEPKAAKNTYTAERFIWLTKLNN
LRILEQGSERPLTDTERATLMDEPYRKSKLTYAQARKLLGLEDTAFFKGLRYGKDNA
EASTLMEMKAYHAISRALEKEGLKDKKSPLNLSSELQDEIGTAFSLFKTDEDITGRLK
DRVQPEILEALLKHISFDKFVQISLKALRRIVPLMEQGKRYDEACAEIYGDHYGKKNT
EEKIYLPPIPADEIRNPVVLRALSQARKVINGVVRRYGSPARIHIETAREVGKSFKDRK
EIEKRQEENRKDREKAAAKFREYFPNFVGEPKSKDILKLRLYEQQHGKCLYSGKEIN
LVRLNEKGYVEIDHALPFSRTWDDSFNNKVLVLGSENQNKGNQTPYEYFNGKDNSR
EWQEFKARVETSRFPRSKKQRILLQKFDEDGFKECNLNDTRYVNRFLCQFVADHILL
TGKGKRRVFASNGQITNLLRGFWGLRKVRAENDRHHALDAVVVACSTVAMQQKIT
RFVRYKEMNAFDGKTIDKETGKVLHQKTHFPQPWEFFAQEVMIRVFGKPDGKPEFE
EADTPEKLRTLLAEKLSSRPEAVHEYVTPLFVSRAPNRKMSGAHKDTLRSAKRFVKH
NEKISVKRVWLTEIKLADLENMVNYKNGREIELYEALKARLEAYGGNAKQAFDPKD
NPFYKKGGQLVKAVRVEKTQESGVLLNKKNAYTIADNGDMVRVDVFCKVDKKGK
NQYFIVPIYAWQVAENILPDIDCKGYRIDDSYTFCFSLHKYDLIAFQKDEKSKVEFAY
YINCDSSNGRFYLAWHDKGSKEQQFRISTQNLVLIQKYQVNELGKEIRPCRLKKRPP
VR

The “e” at the beginning of the Nme2Cas9 variants described herein signify an “evolved” Nme2 variant. Amino acid substitutions relative to wild-type Nme2Cas9 are indicated in bolded underline.

eNme2-C Cas9
(SEQ ID NO: 353)
MAAFKSNPINYILGLDIGIASVGWAMVEIDEEGNPIRLIDLGVRVFERAEVPKTGDSL
AMARRLARSVRRLTRRRAHRLLRARRLLKREGVLQAADFDENGLITSLPNTPWQLR
AAALDRKLTPLEWSAVLLHLIKHRGYLSQRKNEGETAAKELGALLKGVANNAHAL
QTGDFRTPAELALNKFEKESGHIRNQRGDYSHTFSRKDLQAELILLFEKQKEFGNPHV
SGGLKEGIETLLMTQRPALSGDAVQKMLGHCTLEPTEPKAAKNTYTAERFIWLTKL
NNLRILEQGSERPLTDTERSTLMDEPYRKSKLTYAQARKLLGLEDTAFFKGLRYGKD
NAEASTLMEMKAYHAISRALEKEGLKDKKSPLNLSSELQDEIGTAFSLFKTDEDITGR
LKDRVQPEILEALLKHISFDKFVQISLKALRRIVPLMEQGKRYDEACAEIYGVHYGKK
NTEEKIYLPPIPADEIRNPVVLRALSQARKVINGVVRRYGSPARIHIETAREVGKSFKD
RKEIAKRQEENRKDREKAAAKFREYFPNFVGEPKSKDILKLRLYEQQHGKCLYSGKE
INLVRLNEKGYVEIDHALPFSRTWDDSFNNKVLVLGSENQNKGNQTPYEYFNGKDN
SREWQEFKARVETSRFPSSKKQRILLQKFDEDGFKECNLNDTRYVNRFLCQFVADHI
LLTGKGKRRVVASNGQITNLLRGFWRLRKVRAENDRHHALDAVVVACSTVAMQQ
KITRFVRYKEMNAFDGKTVDKETGKVLYQKTHFPQPWEFFAQEVMIRVFGKPDGKP
EFEEADTPEKLRTLLAEKLSSRPEAVHEYVTPLFVSRAPNRKMSGAHKDTLRSAKRF
VKHNEKISVKRVWLTEIKLADLENMVNYKNGREIELYEALKARLEAYGGNAKQAFD
PKDNPFYKKGGQLVKAVRVEKTQKSGVLLNKKNAYTIADNGDMVRVDVFCKVDK
KGKNQYFIVPIYAWQVAENILPDIDCKGYRIDDSYTFCFSLHKYDLIAFQKDEKSKVE
FAYYINCDSSSGGFYLAWHDKGSREQRFRISTQNLALIQKYQVNELGKEIRPCRLKK
RPPVR

In some embodiments, the disclosed base editors comprise a napDNAbp comprising a compact Cas9 ortholog from derived from Campylobacter jejuni (CjCas9). In some embodiments, the napDNAbp comprises CjCas9. In some embodiments, the disclosed base editors comprise a CjCas9 nickase. CjCas9 recognizes recognizes NNNNACA and NNNNACAC PAMs. See Kim et al., Nature Communications 8(14500):1-12 (2017), which is incorporated herein by reference. The sequence of CjCas9 (nickase) is set forth as SEQ ID NO: 348. In some embodiments, the disclosed base editors comprise a napDNAbp domain that has a sequence that is at least 90%, at least 95%, at least 98%, or at least 99% identical to SEQ ID NO: 348. In some embodiments, the disclosed base editors comprise a napDNAbp comprising SEQ ID NO: 348. The length of this protein is 984 amino acids. This protein may be referred to herein as engineered CjCas9, or enCjCas9. The rationally engineered CjCas9 variant (enCjCas9) is described in Nakagawa, et al., Communications Biology, (2022) 5:211, which is herein incorporated by reference. In various embodiments, any of the disclosed TadCBEs comprise a variant of CjCas9 or enCjCas9 (SEQ ID NO: 348).

(SEQ ID NO: 348)
MARILAFAIGISSIGWAFSENDELKDCGVRIFTKVENPKTGESLALPRRLAR
SARKRLARRKARLNHLKHLIANEFKLNYEDYQSFDESLAKAYKGSLISPYELRFRAL
NELLSKQDFARVILHIAKRRGYDDIKNSDDKEKGAILKAIKQNEEKLANYQSVGEYL
YKEYFQKFKENSKEFTNVRNKKESYERCIAQSFLKDELKLIFKKQREFGFSFSKKFEE
EVLSVAFYKRALKDFSHLVGNCSFFTDEKRAPKNSPLAFMFVALTRIINLLNNLKNT
EGILYTKDDLNALLNEVLKNGTLTYKQTKKLLGLSDDYEFKGEKGTYFIEFKKYKE
FIKALGEHNLSQDDLNEIAKDITLIKDEIKLKKALAKYDLNQNQIDSLSKLEFKDHLN
ISFKALKLVTPLMLEGKKYDEACNELNLKVAINEDKKDFLPAFNETYYKDEVTNPV
VLRAIKEYRKVLNALLKKYGKVHKINIELAREVGKNHSQRAKIEKEQNENYKAKK
DAELECEKLGLKINSKNILKLRLFKEQKEFCAYSGEKIKISDLQDEKMLEIDHIYPYS
RSFDDSYMNKVLVFTKQNQEKLNQTPFEAFGNDSAKWQKIEVLAKNLPTKKQKRIL
DKNYKDKEQKNFKDRNLNDTRYIARLVLNYTKDYLDFLPLSDDENTKLNDTQKGS
KVHVEAKSGMLTSALRHTWGFSAKDRNNHLHHAIDAVIIAYANNSIVKAFSDFKKE
QESNSAELYAKKISELDYKNKRKFFEPFSGFRQKVLDKIDEIFVSKPERKKPSGALHE
ETFRKEEEFYQSYGGKEGVLKALELGKIRKVNGKIVKNGDMFRVDIFKHKKTNKFY
AVPIYTMDFALKVLPNKAVARSKKGEIKDWILMDENYEFCFSLYKDSLILIQTKDM
QEPEFVYYNAFTSSTVSLIVSKHDNKFETLSKNQKILFKNANEKEVIAKSIGIQNLKV
FEKYIVSALGEVTKAEFRQREDFKK

The base editors of the present disclosure may also comprise Cas9 variants with modified PAM specificities. Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

In some embodiments, the disclosed base editors comprise a napDNAbp domain comprising a SpCas9-NG, which has a PAM that corresponds to NGN. In some embodiments, the disclosed base editors comprise a napDNAbp domain that has a sequence that is at least 90%, at least 95%, at least 98%, or at least 99% identical to SpCas9-NG. The sequence of SpCas9-NG is illustrated below:

(SEQ ID NO: 356)
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESIRPKRNSDKLIARKKDWDPKKYGGFVSPTVAYSVLVV
AKVEKGKSKKLKSVKELLGITIMERSSFEKNPIDFLEAKGYKEVKKDLIIKLPKYSLFE
LENGRKRMLASARFLQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PRAFKYFDTTIDRKVYRSTKEVLDATLIHQSITGLYETRIDLSQLGGD

In some embodiments, the disclosed base editors comprise a napDNAbp domain comprising a S. aureus Cas9 nickase KKH, or SaCas9-KKH or SaKKH-Cas9, which has a PAM that corresponds to NNNRRT, or NNGRRT. This Cas9 variant contains the amino acid substitutions D10A, E782K, N968K, and R1015H (“KKH”) relative to wild-type SaCas9, set forth as SEQ ID NO: 347. In some embodiments, the disclosed base editors comprise a napDNAbp domain that has a sequence that is at least 90%, at least 95%, at least 98%, or at least 99% identical to SaCas9-KKH. The length of SaCas9 (and SaKKH-Cas9) is 1053 amino acids. The sequence of SaCas9-KKH (nickase) is illustrated below:

S. aureus Cas9 nickase KKH (SaCas9-KKH)
(SEQ ID NO: 357)
MGKRNYILGLAIGITSVGYGIIDYETRDVIDAGVRLFKEANVENNEGRRSKRGARRL
KRRRRHRIQRVKKLLFDYNLLTDHSELSGINPYEARVKGLSQKLSEEEFSAALLHLAK
RRGVHNVNEVEEDTGNELSTKEQISRNSKALEEKYVAELQLERLKKDGEVRGSINRF
KTSDYVKEAKQLLKVQKAYHQLDQSFIDTYIDLLETRRTYYEGPGEGSPFGWKDIKE
WYEMLMGHCTYFPEELRSVKYAYNADLYNALNDLNNLVITRDENEKLEYYEKFQII
ENVFKQKKKPTLKQIAKEILVNEEDIKGYRVTSTGKPEFTNLKVYHDIKDITARKEIIE
NAELLDQIAKILTIYQSSEDIQEELTNLNSELTQEEIEQISNLKGYTGTHNLSLKAINLIL
DELWHTNDNQIAIFNRLKLVPKKVDLSQQKEIPTTLVDDFILSPVVKRSFIQSIKVINAI
IKKYGLPNDIIIELAREKNSKDAQKMINEMQKRNRQTNERIEEIIRTTGKENAKYLIEKI
KLHDMQEGKCLYSLEAIPLEDLLNNPFNYEVDHIIPRSVSFDNSFNNKVLVKQEENSK
KGNRTPFQYLSSSDSKISYETFKKHILNLAKGKGRISKTKKEYLLEERDINRFSVQKDF
INRNLVDTRYATRGLMNLLRSYFRVNNLDVKVKSINGGFTSFLRRKWKFKKERNKG
YKHHAEDALIIANADFIFKEWKKLDKAKKVMENQMFEEKQAESMPEIETEQEYKEIF
ITPHQIKHIKDFKDYKYSHRVDKKPNRKLINDTLYSTRKDDKGNTLIVNNLNGLYDK
DNDKLKKLINKSPEKLLMYHHDPQTYQKLKLIMEQYGDEKNPLYKYYEETGNYLTK
YSKKDNGPVIKKIKYYGNKLNAHLDITDDYPNSRNKVVKLSLKPYRFDVYLDNGVY
KFVTVKNLDVIKKENYYEVNSKCYEEAKKLKKISNQAEFIASFYKNDLIKINGELYRV
IGVNNDLLNRIEVNMIDITYREYLENMNDKRPPHIIKTIASKTQSIKKYSTDILGNLYE
VKSKKHPQIIKKG

In some embodiments, the disclosed base editors comprise a napDNAbp comprising a Cas9 protein derived from Staphylococcus auricularis (S. auri Cas9, or SauriCas9). In some embodiments, the disclosed base editors comprise a SauriCas9 nickase. SauriCas9 recognizes NNGG and NNNGG PAMs. The sequence of SauriCas9 (nickase) is set forth as SEQ ID NO: 358. In some embodiments, the disclosed base editors comprise a napDNAbp domain that has a sequence that is at least 90%, at least 95%, at least 98%, or at least 99% identical to SEQ ID NO: 358. In some embodiments, the disclosed base editors comprise a napDNAbp comprising SEQ ID NO: 358. The length of this protein is 1061 amino acids.

(SEQ ID NO: 358)
MQENQQKQNYILGLAIGITSVGYGLIDSKTREVIDAGVRLFPEADSENNSNRRSKRGA
RRLKRRRIHRLNRVKDLLADYQMIDLNNVPKSTDPYTIRVKGLREPLTKEEFAIALLH
IAKRRGLHNISVSMGDEEQDNELSTKQQLQKNAQQLQDKYVCELQLERLTNINKVR
GEKNRFKTEDFVKEVKQLCETQRQYHNIDDQFIQQYIDLVSTRREYFEGPGNGSPYG
WDGDLLKWYEKLMGRCTYFPEELRSVKYAYSADLFNALNDLNNLVVTRDDNPKLE
YYEKYHIIENVFKQKKNPTLKQIAKEIGVODYDIRGYRITKSGKPQFTSFKLYHDLKNI
FEQAKYLEDVEMLDEIAKILTIYQDEISIKKALDQLPELLTESEKSQIAQLTGYTGTHR
LSLKCIHIVIDELWESPENQMEIFTRLNLKPKKVEMSEIDSIPTTLVDEFILSPVVKRAFI
QSIKVINA VINRFGLPEDIIIELAREKNSKDRRKFINKLQKQNEATRKKIEQLLAKYGN
TNAKYMIEKIKLHDMQEGKCLYSLEAIPLEDLLSNPTHYEVDHIIPRSVSFDNSLNNK
VLVKQSENSKKGNRTPYQYLSSNESKISYNQFKQHILNLSKAKDRISKKKRDMLLEE
RDINKFEVQKEFINRNLVDTRYATRELSNLLKTYFSTHDYAVKVKTINGGFTNHLRK
VWDFKKHRNHGYKHHAEDALVIANADFLFKTHKALRRTDKILEQPGLEVNDTTVK
VDTEEKYQELFETPKQVKNIKQFRDFKYSHRVDKKPNRQLINDTLYSTREIDGETYV
VQTLKDLYAKDNEKVKKLFTERPQKILMYQHDPKTFEKLMTILNQYAEAKNPLAAY
YEDKGEYVTKYAKKGNGPAIHKIKYIDKKLGSYLDVSNKYPETQNKLVKLSLKSFRF
DIYKCEQGYKMVSIGYLDVLKKDNYYYIPKDKYEAEKQKKKIKESDLFVGSFYYND
LIMYEDELFRVIGVNSDINNLVELNMVDITYKDFCEVNNVTGEKRIKKTIGKRVVLIE
KYTTDILGNLYKTPLPKKPQLIFKRGEL

In some embodiments, the napDNAbp comprises a SauriCas9-KKH variant, or a SauriCas9-KKH nickase variant. SauriCas9-KKH contains corresponding triple KKH mutations: Q788K, Y973K, and R1020H. See Hu et al. (2020) PLoS Biol. 18(3): e3000686, which is incorporated herein by reference.

In some embodiments, the disclosed base editors comprise a napDNAbp domain comprising an S. pyogenes Cas9 nickase KKH, or SpCas9-KKH, which has a PAM that corresponds to NNNRRT.

In some embodiments, the Cas variant is a variant of SpRY that has mutations conferring high fidelity. Such a variant is known as SpRY-HF or SpRY-HF1. High-fidelity variants of SpRY, or any of the Cas variants provided herein, may comprise one or more of N497A, R661A, Q695A, and/or Q926A mutation of relative to the SEQ ID NO: 74, or a corresponding mutation in any Cas9 provided herein. Cas9 variants with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); each of which is incorporated herein by reference.

In some embodiments, the disclosed Cas variants include variants of a Cas9 derived from a Streptococcus macacae, e.g. Streptococcus macacae NCTC 11558, or SmacCas9. In some embodiments, the Cas variant comprises a hybrid variant of SmacCas9 that incorporates an SpCas9 domain with the SmacCas9 domain and is known as Spy-macCas9, or a variant thereof. In some embodiments, the Cas variant comprises a hybrid variant of SmacCas9 that incorporates an increased nucleolytic variant of an SpCas9 (iSpy Cas9) domain and is known as iSpy-macCas9. Relative to Spymac-Cas9, iSpyMac-Cas9 contains two mutations, R221K and N394K, that were identified by deep mutational scans of Spy Cas9 that raise modification rates of the protein on most targets. See Jakimo et al., bioRxiv, A Cas9 with Complete PAM Recognition for Adenine Dinucleotides (September 2018), herein incorporated by reference. Jakimo et al. showed that the hybrids Spy-macCas9 and iSpy-macCas9 recognize a short 5′-NAA-3′ PAM and recognized all evaluated adenine dinucleotide PAM sequences and possesses robust editing efficiency in human cells. Liu et al. engineered base editors containing Spy-mac Cas9, and demonstrated that cytidine and adenine base editors containing Spymac domains can induce efficient C-to-T and A-to-G conversions in vivo. In addition, Liu et al. suggested that the PAM scope of Spy-mac Cas9 may be 5′-TAAA-3′, rather than 5′-NAA-3′ as reported by Jakimo et al (see Liu et al. Cell Discovery (2019) 5:58, herein incorporated by reference).

Any of the references noted above which relate to Cas9 variants or Cas9 equivalents are hereby incorporated by reference in their entireties, if not already stated so.

The following table provides a comparison of the PAM preferences targeted by the presently disclosed Nme2Cas variants to those targeted by other Cas homologs (R=any purine, Y=any pyrimidine, N=any nucleotide).

TABLE 6
PAM preferences of Exemplary Cas homologs

| Cas homolog | PAM preference(s) |
|---|---|
| S. pyogenes Cas9 (SpCas9) | NGG [canonical] |
| WT Nme1Cas9 | N₄GAYW/N₄GYTT/N₄GTCT |
| WT Nme2Cas9 | NNNNCC or NNNNGATT |
| eNme2-C Cas9 | NNNNCN |
| SaCas9 | NNGRRT/NGRRT or NNGRRN/NGRRN |
| SaCas9-KKH | NNNRRT or NNGRRT |
| SauriCas9 | NNGG |
| CjCas9 | NNNNACA, NNNNRYAC, or NNNNACAC |
| Cas homolog | PAM preference(s) |
| StCas9 | NNAGAAW |
| SpRY | NYN |
| SmacCas9 | NAA/TAAA |
| SpCas9-VRQR | NGAN or NGNG |
| SpCas9-EQR | NGAG |
| SpCas9-VRER | NGCG |

### PAM/Protospacer Sequences

Base editing requires the presence of a protospacer adjacent motif (PAM) located approximately 15 base pairs from the target nucleotide(s) for canonical (i.e., S. pyogenes Cas9-derived) base editors. Each programmable DNA-binding protein domain recognizes a different PAM sequence. Only about one quarter of pathogenic transition point mutations have a suitably located canonical PAM “NGG” sequence that is compatible with S. pyogenes Cas9 (SpCas9)-derived base editors. Naturally-occurring cytidine deaminases have shown broad compatibility with many Cas homologs, including S. aureus Cas9 (SaCas9)⁹⁸, SaCas9-KKH⁸, Cas12a (Cpf1)^{9,10}, SpCas9-NG¹¹, and circularly permuted CP-Cas9s⁷, greatly expanding their targeting scope.

In some embodiments, the napDNAbp comprises a PAM sequence and a protospacer located upstream of the PAM sequence. In some embodiments, the protospacer sequence is upstream of a PAM with the sequence TGG. In other embodiments, the protospacer sequence is upstream of a PAM with the sequence GGG. In yet other embodiments, the protospacer sequence is upstream of a PAM with the sequence AGG. In some embodiments, the protospacer sequence is upstream of a PAM with the sequence CGG. In some embodiments, the protospacer sequence is upstream of a PAM with the sequence AGACCC. In other embodiments, the protospacer sequence is upstream of a PAM with the sequence ACCTCA. In some embodiments, the protospacer sequence is upstream of a PAM with the sequence GGGGCG. In other embodiments, the protospacer sequence is upstream of a PAM with the sequence CAGCCG. In some embodiments, the protospacer sequence is upstream of a PAM with the sequence GCGGCT. In yet other embodiments, the protospacer sequence is upstream of a PAM with the sequence GGGGCA. In some embodiments, the protospacer sequence is upstream of a PAM with the sequence AAGGGT. In other embodiments, the protospacer sequence is upstream of a PAM with the sequence TCGGGT. In some embodiments, the protospacer sequence is upstream of a PAM with the sequence GAGAGT. In some embodiments, the protospacer sequence is upstream of a PAM with the sequence CAGAAT. In some embodiments, the protospacer sequence is upstream of a PAM with the sequence CTGGGT.

In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the target region comprises a target window, wherein the target window comprises the target nucleobase pair.

Protospacer sequences of the present disclosure may include, but are not limited to, the following sequences:

| Sequence | SEQ ID NO: |
|---|---|
| GGAATCCCTTCTGCAGCACC | 208 |
| GAGTCCGAGCAGAAGAAGAA | 209 |
| TTTATCACAGGCTCCAGGAA | 210 |
| GGCCCAGACTGAGCACGTGA | 211 |
| ACTCACGCTGGATAGCCTCC | 212 |
| GTCATCTTAGTCATTACCTG | 213 |
| GGCACTGCGGCTGGAGGTGG | 214 |
| CACCTACCTAAGAACCATCC | 215 |
| TTCGTATCTGTAAAACCAAG | 216 |
| CAATGTGTCAACTCTTGACA | 217 |
| CAGTTTCAGCACATCATGGT | 218 |
| GGTCCAGGCGAACGCGAAGT | 219 |
| GGAATCCCTTCTGCAGCACC | 208 |
| GAGTCCGAGCAGAAGAAGAA | 209 |
| TTTATCACAGGCTCCAGGAA | 210 |
| GGCCCAGACTGAGCACGTGA | 211 |
| ACTCACGCTGGATAGCCTCC | 212 |
| GTCATCTTAGTCATTACCTG | 213 |
| GCAAGAGCACAAGAGGAAGAGAG | 226 |
| GGCTACAGCAACAGGGTGGTGG | 227 |
| GGTGTGCAGACGGCAGTCACTAG | 228 |
| GTCTCCGCTTTAACCCCCACCTC | 229 |
| CCTCTTGCCTCCACTGGTTGTGC | 230 |
| GGTCCAAAGCAGGATGACAGGCA | 231 |
| CACCCAGACTGAGCACGTGC | 232 |
| GACACAGACTGGGCACGTGA | 233 |
| AGCTCAGACTGAGCAAGTGA | 234 |
| AGACCAGACTGAGCAAGAGA | 235 |
| GAGCCAGAATGAGCACGTGA | 236 |
| TGCACTGCGGCCGGAGGAGG | 237 |
| GGCTCTGCGGCTGGAGGGGG | 238 |
| GGCACGACGGCTGGAGGTGG | 239 |
| GGCATCACGGCTGGAGGTGG | 240 |
| GGCGCTGCGGCGGGAGGTGG | 241 |
| GAGTTAGAGCAGAAGAAGAA | 242 |
| GAGTCTAAGCAGAAGAAGAA | 243 |
| GAGGCCGAGCAGAAGAAAGA | 244 |
| GAGTCCTAGCAGGAGAAGAA | 245 |
| AAGTCTGAGCACAAGAAGAA | 246 |
| GAGTCCGGGAAGGAGAAGAA | 247 |
| GAGCCGGAGCAGAAGAAGGA | 248 |
| AAGTCCGAGGAGAGGAAGAA | 249 |
| GAATCCAAGCAGGAGAAGAA | 250 |
| ACGTCTGAGCAGAAGAAGAA | 251 |
| CCTATCACTGGCTCCAGGAA | 252 |
| CTTATCATAGGCCCCAGGAA | 253 |
| TAGGGTGTCAACTCTTGACA | 254 |
| TTATCTGTCAACTCTTGACA | 255 |
| ATACCTGCCAACTCTTGACA | 256 |
| TGTTCTCAGCACATCATGGT | 257 |
| GTGGTAGACAGCATGTGTCCTA | 258 |
| ATTTACAGCCTGGCCTTTGGGG | 259 |
| GTGTCAGGTAATGTGCTAAACA | 260 |
| GGTGGAGGAGGGTGCATGGGGT | 261 |
| TCTGCTTCTCCAGCCCTGGC | 262 |
| GATGTTCCAATCAGTACGCA | 263 |

### Editing Window

The base editors of the present disclosure may possess variable target regions of a target window (e.g., editing window, or deamination window) comprising a target nucleobase pair within which a nucleotide change is installed. In some embodiments, a TadA-CD has a C-to-T base editing window that corresponds to protospacer positions 2-12 of the protospacer. In some embodiments, the TadA-CD base editor has a C-to-T base editing window that corresponds to protospacer positions 2-12. In particular embodiments, the TadA-CD base editor has a C-to-T base editing window that corresponds to protospacer positions 3 to 8. The base editors of this disclosure may have particularly high editing activity on cytosines between protospacer positions 5 to 7.

In some embodiments, the target window (e.g., editing window) comprises 1-10 nucleotides. In some embodiments, the editing window is 1-9, 1-8, 1-7, 1-6, 1-5, 1-4, 1-3, 1-2, or 1 nucleotides in length. In some embodiments, the target window (e.g., editing window) is 1, 2, 3, 4, 5, 6, 7, 8, 9, 10, 11, 12, 13, 14, 15, 16, 17, 18, 19, or 20 nucleotides in length. In some embodiments, the intended edited base pair is within the editing window. In some embodiments, the editing window comprises the intended edited base pair.

In certain cases the TadA-CD base editing window starts after position 2, after position 3, after position 4, after position 5, after position 6, after position 7, after position 8, after position 9, after position 10, and after position 11 of the protospacer. In some embodiments, the editing window ends before position 12, before position 11, before position 10, before position 9, before position 8, before position 7, before position 6, before position 5, before position 4, and before position 3 of the protospacer.

In some embodiments, TadA-CD base editors comprising a V106W mutation have narrower editing windows relative to TadA-CD base editors lacking said mutation. For instance, the base editing window of TadA-CDa (SEQ ID NO: 34) is between ˜position 4 and ˜position 9 of the protospacer. In certain embodiments, TadA-CD base editors comprising a V106W mutation (e.g., TadA-CDa V106W and TadA-CDd V106W), possess a C-to-T base editing window between position 3 and position 9 of the protospacer, or any combination thereof. For example, the editor may install a C-to-T substitution at position 3, position 4, position 5, position 6, position 7, position 8, or position 9 of the protospacer, or any combination thereof.

In some cases, the TadA-CD V106W base editing window starts after position 2, after position 4, after position 5, after position 6, after position 7, after position 8, or after position 9 of the protospacer. In some embodiments, the editing window ends before position 10, before position 9, before position 8, before position 7, before position 6, before position 5, before position 4 of the protospacer.

In some embodiments, the TadA-CD base editor has an A-to-G base editing window of between about position 4 and position 7 of the protospacer. In some cases, the TadA-CD base editor installs an A-to-G edit at position 4, position 5, position 6, or position 7 of the protospacer, or any combination thereof. The A-to-G base editing properties of TadA-CDs, according to some embodiments, may be narrowed to between position 5 and position 7 of the protospacer, by including a V106W mutation.

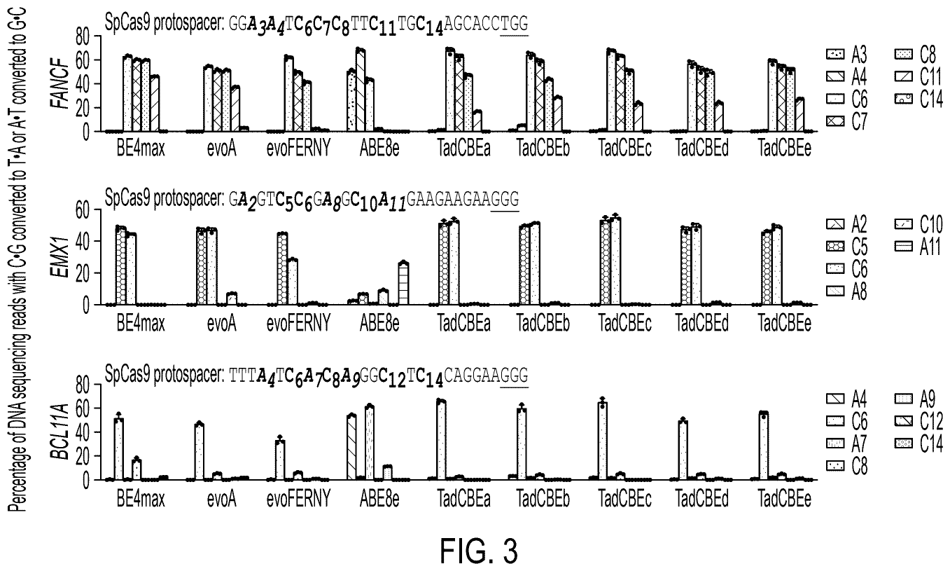

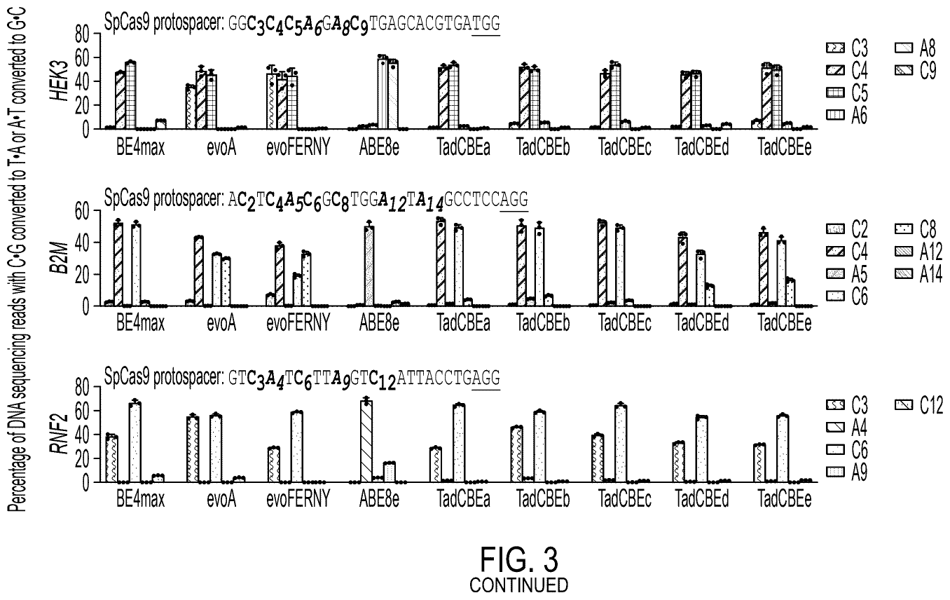

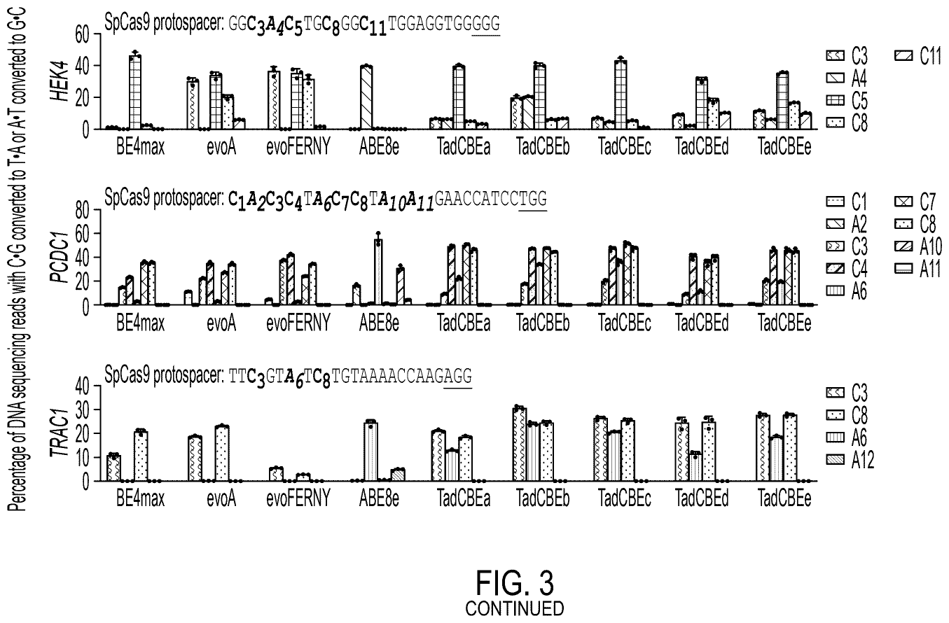

Those of skill in the art will appreciate that the TadA-CD base editors described above and herein, have narrower C-to-T base editing windows than several existing cytidine deaminases, such as rAPOBEC1, evoAPOBEC1 (evoA), evoFERNY, and YE1. For example, BE4max and evoABE4max exhibit C-to-T editing windows ranging from position 1 to position 14 of the protospacer; evoFERNY-BE4 exhibits C-to-T editing windows from position 1 to about position 10; and YE1-BE4 exhibits C-to-T editing windows from position 3 to position 9 of the protospacer (see FIG. 3).

Those of skill in the art will also appreciate that the TadA-CD base editors described above and herein, possess narrower A-to-G and wider C-to-T base editing windows compared to the parent adenosine deaminase from which it was evolved (e.g., TadA-8e). For instance, TadA-8e exhibits an A-to-G base editing window of between position 1 and position 15 of the protospacer and a C-to-T base editing window of between position 4 to position 7 of the protospacer.

TadA-CD base editors of the present disclosure may convert one or more target cytosines to thymines within the protospacer sequence. For example, in some embodiments, the TadA-CB may convert 2 cytosines, 3 cytosines, 4 cytosines, or 5 cytosines within a protospacer sequence.

### Editing Efficiencies

Aspects of the disclosure relate to the efficiency of the cytosine base editors, as described herein, to edit a DNA target sequence within a target region of a target window comprising a target nucleobase pair. In some embodiments, at least 10%, 15%, 20%, 25%, 30%, 35%, 40%, 45%, or 50% of the intended base pairs are edited. In some embodiments, the efficiency of C-to-T conversion of any of the disclosed base editors or methods of using these base editors is at least 80%, over all sequencing reads. In particular embodiments, TadCBEa achieved an average of 51-60% conversion efficiency of target cytosines.

In some embodiments, any of the disclosed base editors or methods of using these base editors provides an average of 70% cytosine conversion efficiency in clinically-relevant genes such as the CXCR5 and CCR5 genes, which are implicated in HIV/AIDS.

In some embodiments, the cytidine deamination activity of the disclosed deaminases (and thus the cytosine editing activity of the disclosed base editors) exceeds the adenosine deamination activity of the deaminase by a significant ratio. For example, the ratio of the cytidine deamination activity to the adenosine deamination activity of the disclosed Tad-CD deaminases is at least about 5:1, 6:1, 7:1, 8:1, 9:1, 10:1, 11:1, 12:1, 13:1, 14:1, 15:1, 17:1, 19:1, 20:1, 21:1, 23:1, 25:1, 30:1, or greater than 30:1. In some embodiments, the ratio of the cytidine deamination activity to the adenosine deamination activity of the deaminase is at least about 10:1. In some embodiments, the ratio is at least about 20:1. In some embodiments, the ratio is about 5:1-7.5:1, 7.5:1-9.5:1, 5:1-10:1, 10:1-15:1, 15:1-20:1, 10-17:1, 12:1-17:1, 20:1-21:1, 21:1-25:1, 20:1-30:1, 25:1-35:1, 30:1-35:1, 30:1-40:1, 40:1-42:1, 21:1-42:1, 25:1-40:1, 10:1-40:1, 25-45:1, 30:1-50:1, 45:1-50:1, 50:1-60:1, 55:1-65:1, 60:1-70:1, 70:1-80:1, 80:1-85:1, 10:1-80:1, 40:1-80:1, 20:1-60:1, 20:1-80:1, or 75:1-85:1.

In some embodiments, the peak editing efficiency of TadA-CDs is comparable to native cytosine base editors (e.g., BE4max editors containing APOBEC1, evoFERNY, or evoA deaminases). In some embodiments, the editing efficiency of TadA-CD base editors is higher relative to native cytosine base editors. For example, in some embodiments, TadA-CDa, TadA-CDb, and TadA-CDc edit the Nme50 gene at positions 3-8 of the protospacer with between 5 and 48% efficiency. In some instances, the TadA-CDs comprise a V106W substitution that maintains the editing efficiency while narrowing the editing window of the base editors.

In some embodiments, the disclosed TadCBEs and editing methods comprising the step of contacting a DNA with any of the disclosed TadCBEs result in an on-target DNA (C-to-T) base editing efficiency of at least about 20%, 21%, 25%, 30%, 35%, 40%, 50%, 60%, 70%, 80%, 85%, or more than 85% at the target nucleobase pair, over all sequencing reads. The step of contacting may result in a C-to-T base editing efficiency of at least about 40%, 41%, 42%, 43%, 44%, 45%, 46%, 47%, 48%, 49%, 50%, 52%, 55%, 60%, 62%, 65%, 70%, 72%, 75%, 80%, 82%, 85%, or more than 85%. In particular, the step of contacting results in on-target base editing efficiencies of greater than 75%. In certain embodiments, base editing efficiencies of 99% may be realized.

In some cases, the TadA-CD base editors described herein have a C-to-T editing efficiency of between 20% and 80%. In some embodiments, the C-to-T editing efficiency is greater than or equal to 10%, 20%, 25%, 30%, 40%, 45%, 50%, 55%, 60%, 65%, 70%, 75%, or 80%. In other embodiments, the C-to-T editing efficiency is less than or equal to 95%, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1%.

The base editors of the present disclosure, may in some cases, possess varying base editing efficiencies (e.g., converting a C to T) of targeted nucleotides within a given protospacer sequence. In other words, the TadA-CD base editors of the current invention may preferentially edit a certain position (or positions) within the protospacer sequence. For instance, in some embodiments, the TadA-CDa variant preferentially edits the C8 position of the protospacer sequence GC₂A₃A₄GA₆GC₈A₉C₁₀A₁₁A₁₂GAGGAAGAGAGAGACCC (SEQ ID NO: 385), where the PAM is underlined; whereas the TadA-CDc variant edits both C8 and C10 positions with similar efficiencies.

Thus, in some embodiments, the TadCBE editing efficiency at each position of the protospacer (e.g., position 1 through position 15) within the editing window is between 20% and 80%. In some embodiments, the editing efficiency at each position of the protospacer (e.g., position 1 through position 15) within the editing window is greater than or equal to 10%, greater than or equal to 20%, greater than or equal to 30%, greater than or equal to 40%, greater than or equal to 50%, greater than or equal to 60%, greater than or equal to 70%, greater than or equal to 80%, or greater than or equal to 85%. In other embodiments, the editing efficiency at each position of the protospacer (e.g., position 1 through position 15) within the editing window is less than or equal to 85%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1%.

Accordingly, in some embodiments, the TadCBEs of the instant application provide an efficiency of conversion of a C-to-T base of at least 20%, 21%, 25%, 30%, 35%, 40%, 41%, 42%, 43%, 44%, 45%, 46%, 47%, 48%, 49%, 50%, 52%, 55%, 60%, 62%, 65%, 70%, 72%, 75%, 80%, 82%, 85%, or more than 85% when contacted with a DNA comprising a target sequence selected from the group consisting of CTT, CTC, CTA, CTG, CCT, CCC, CCA, CCG, CAT, CAC, CAA, CAG, CGT, CGC, CGA, CGG, TCT, TCC, TCG, ACT, ACC, ACA, ACG, GCT, GCC, GCA, GCG, TTC, TAC, TGC, ATC, AAC, AGC, GTC, GAC, and GGC.

The disclosed TadCBEs possess greater affinity and specificity for cytosine bases, and therefore are less prone to deaminate adenosine residues. In some cases, the TadA-CD base editors described herein have a low residual A-to-G editing efficiency, e.g., of between 0.1% and 20%. In some cases, the TadA-CD base editors described herein have a low residual A-to-G editing efficiency, e.g., of between 0.1% and 20%. In some embodiments, the A-to-G editing efficiency is greater than or equal to 0.1%, greater than or equal to 5%, greater than or equal to 10%, or greater than or equal to 20%. In other embodiments, the A-to-G editing efficiency is less than or equal to 95%, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 0.1%.

In some cases, the TadA-CD base editors described herein have residual (off-target) C-to-G editing capability. In some embodiments, V106W variants have reduced C-to-G editing compared to native TadA-CD base editors. In some embodiments, the TadA-CD V016W mutants reduce C-to-G editing in HEK293T cells, T cells, and HSPCs.

### Guide Sequences

The present disclosure further provides guide RNAs for use in accordance with the disclosed methods of editing. The disclosure provides guide RNAs (gRNAs) that are designed to recognize target sequences. Such gRNAs may be designed to have guide sequences (or “spacers”) having complementarity to a protospacer within the target sequence.

Guide RNAs are also provided for use with one or more of the disclosed adenine base editors, e.g., in the disclosed methods of editing a nucleic acid molecule. Such gRNAs may be designed to have guide sequences having complementarity to a protospacer within a target sequence to be edited, and to have backbone sequences that interact specifically with the napDNAbp domains of any of the disclosed base editors, such as Cas9 nickase domains of the disclosed base editors.

In various embodiments, the base editors may be complexed, bound, or otherwise associated with (e.g., via any type of covalent or non-covalent bond) one or more guide sequences. The guide sequence becomes associated or bound to the base editor and directs its localization to a specific target sequence having complementarity to the guide sequence or a portion thereof. The particular design embodiments of a guide sequence will depend upon the nucleotide sequence of a genomic target sequence (i.e., the desired site to be edited) and the type of napDNAbp (e.g., type of Cas9 protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of the napDNAbp (e.g., a Cas9 or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, 80, 85, 90, 95, 100 or more nucleotides in length. In other embodiments, the guide sequence is about or more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, or 200 nucleotides long. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37,38, 39, or 40 contiguous nucleotides) that is complementary to a target sequence (or off-target site). In some embodiments, each gRNA comprises a guide sequence of at least 15 contiguous nucleotides that is complementary to a target sequence (or off-target site). In other embodiments, each gRNA comprises a guide sequence of at least 20 contiguous nucleotides that is complementary to a target sequence (or off-target site).

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence. Similarly, cleavage of a target polynucleotide sequence may be evaluated in situ by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and P A Carr & G M Church, 2009, Nature Biotechnology 27(12): 1151-62). Additional algorithms may be found in Chuai, G. et al., DeepCRISPR: optimized CRISPR guide RNA design by deep learning, Genome Biol. 19:80 (2018), and U.S. Application Ser. No. 61/836,080 and U.S. Pat. No. 8,871,445, issued Oct. 28, 2014, the entireties of each of which are incorporated herein by reference.

The guide sequence of the gRNA is linked to a tracer mate (also known as a “backbone”) sequence which in turn hybridizes to a tracr sequence. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides.

Non-limiting examples of single (DNA) polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 264)
NNNNNNNNgtttttgtactctcaagatttaGAAAtaaatcttgcagaag
ctacaaagataaggcttcatgccgaaatcaacaccctgtcattttatgg
cagggtgttttcgttatttaaTTTTTT;
(2)
(SEQ ID NO: 265)
NNNNNNNNNNNNNNNNNNgtttttgtactctcaGAAAtgcagaagctac
aaagataaggcttcatgccgaaatcaacaccctgtcattttatggcagg
gtgttttcgttatttaaTTTTTT;
(3)
(SEQ ID NO: 266)
NNNNNNNNNNNNNNNNNNNNgtttttgtactctcaGAAAtgcagaagct
acaaagataaggcttcatgccgaaatcaacaccctgtcattttatggca
gggtgtTTTTT;
(4)
(SEQ ID NO: 267)
NNNNNNNNNNNNNNNNNNNNgttttagagctaGAAAtagcaagttaaaa
taaggctagtccgttatcaacttgaaaaagtggcaccgagtcggtgcTT
TTTT;
(5)
(SEQ ID NO: 268)
NNNNNNNNNNNNNNNNNNNgttttagagctaGAAATAGcaagttaaaat
aaggctagtccgttatcaacttgaaaaagtgTTTTTTT;
and
(6)
(SEQ ID NO: 269)
NNNNNNNNNNNNNNNNNNNNgttttagagctagAAATAGcaagttaaaa
taaggctagtccgttatcaTTTTTTTT.

In some embodiments, sequences (1) to (3) are used in combination with Cas9 from S. thermophiles CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, the guide RNAs for use in accordance with the disclosed methods of editing comprise synthetic single guide RNAs (sgRNAs) containing modified ribonucleotides. In some embodiments, the guide RNAs contain modifications such as 2′-O-methylated nucleotides and phosphorothioate linkages. In some embodiments, the guide RNAs contain 2′-O-methyl modifications in the first three and last three nucleotides, and phosphorothioate bonds between the first three and last three nucleotides. Exemplary modified synthetic sgRNAs are disclosed in Hendel A. et al., Nat. Biotechnol. 33, 985-989 (2015), herein incorporated by reference.

In some embodiments, the guide RNAs for use in accordance with the disclosed methods of editing comprise a backbone structure that is recognized by an S. pyogenes Cas9 protein or domain, such as an SpCas9 domain of the disclosed base editors. The backbone structure recognized by an SpCas9 protein may comprise the sequence 5′-[guide sequence]-guuuuagagcuagaaauagcaaguuaaaauaaggcuaguccguuaucaacuugaaaaaguggcaccgagucggugcuu uuu-3′ (SEQ ID NO: 339), wherein the guide sequence comprises a sequence that is complementary to the protospacer of the target sequence. See U.S. Publication No. 2015/0166981, published Jun. 18, 2015, the disclosure of which is incorporated by reference herein. The guide sequence is typically 20 nucleotides long.

In other embodiments, the guide RNAs for use in accordance with the disclosed methods of editing comprise a backbone structure that is recognized by an S. aureus Cas9 protein. The backbone structure recognized by an SaCas9 protein may comprise the sequence 5′-[guide sequence]-guuuuaguacucuguaaugaaaauuacagaaucuacuaaaacaaggcaaaaugccguguuuaucucgucaacuuguug gcgagauuuuuuu-3′ (SEQ ID NO: 78).

In some embodiments, the guide RNAs for use in accordance with the disclosed methods of editing comprise a backbone structure that is recognized by an N. meningitis Cas9 protein or domain, such as an Nme2Cas9 domain. The backbone structure (or scaffold) recognized by an Nme2Cas9 protein may comprise the sequence provided below: 5′-[guide sequence]-gttgtagctccctttctcatttcggaaacgaaatgagaaccgttgctacaataaggccgtctgaaaagatgtgccgcaacgctctgccc cttaaagcttctgctttaaggggcatcgttta-3′ (SEQ ID NO: 274). This scaffold sequence is recognized by the Nme1Cas9, Nme2Cas9, and Nme3Cas9 proteins.

The sequences of suitable guide RNAs for targeting the disclosed TadCBEs to specific genomic target sites will be apparent to those of skill in the art based on the present disclosure. Such suitable guide RNA sequences typically comprise guide sequences that are complementary to a nucleic sequence within 50 nucleotides upstream or downstream of the target nucleobase pair to be edited. Some exemplary guide RNA sequences suitable for targeting any of the provided TadCBEs to specific target sequences are provided herein. Additional guide sequences are well known in the art and may be used with the base editors described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are incorporated herein by reference.

### Complexes

Aspects of the current disclosure relate to a complex comprising any of the base editors and a guide RNA bound to any of the napDNAbp domains, as described herein. In some embodiments, the complex comprises a guide RNA that is from about 15-100 nucleotides long and comprises a sequence of at least 10, at least 15, or at least 20 contiguous nucleotides that is complementary to a target sequence. In some cases, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In certain embodiments, the guide RNA is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, or 200 nucleotides long

In some embodiments, the complex comprises a target sequence comprising a DNA sequence. In some cases, the target sequence is in the genome of an organism. The organism, may be, in some embodiments a prokaryote or a eukaryote. The organism may, in some embodiments, be any type of prokaryote or eukaryote known to those of skill in the art. For example, in some embodiments the prokaryote is a bacteria and the eukaryote is a plant for fungus. In some cases, however, the eukaryote may be a vertebrate or a mammal. The mammal, may be for example, a rodent or a human, according to certain embodiments.

The complex, in certain embodiments, comprises a target sequence that is in the genome of a cell. The cell may arise from any origin (e.g., prokaryote or eukaryote) and be of any classification (e.g., muscle cells, skin cell, heart cells, liver cells, etc.) known to those of ordinary skill in the art. Exemplary embodiments include, but are not limited to, mouse cells, rat cells, or human cells. In some embodiments, the cell is a T-cell or a hematopoietic stem cell.

### TadA-Derived Cytosine Base Editors

Aspects of this disclosure relate to novel cytosine base editors (TadCBEs) comprising TadA-derived cytidine deaminases (e.g, TadA-CDs) fused to a nucleic acid programmable DNA binding protein (napDNAbp domain) (e.g., Cas9 domains). Any of the disclosed cytosine base editors may comprise one or more linkers. Any of the disclosed cytosine base editors may comprise one or more UGI domains, e.g., two UGI domains. Any of the disclosed cytosine base editors may comprise one or more linkers. Any of the disclosed cytosine base editors may comprise one or more nuclear localization sequences (NLSs).

The disclosed novel TadCBEs comprise novel combinations of TadA-derived cytidine deaminases, such as the TadA-CDa, TadA-CDc, TadA-CDd, and TadA-CDd (V106W) deaminases, napDNAbp domains, one or more uracil glycosylase inhibitor (UGI) domains and nuclear localizations sequence (NLS) domains, relative to existing base editors.

The disclosed TadCBEs may further comprise one or more nuclear localization signals (NLSs) and/or two uracil glycosylase inhibitor (UGI) domains. Thus, the base editors may comprise the structure: NH₂-[first nuclear localization sequence]-[TadA-CD domain]-[napDNAbp domain]-[first UGI domain]-[second UGI domain]-[second nuclear localization sequence]-COOH, wherein each instance of “]-[” indicates the presence of an optional linker sequence. Exemplary TadCBEs may have a structure that comprises the “BE4max” architecture, with an NH₂-[NLS]-[TadA-CD domain]-[Cas9 nickase domain]-[UGI domain]-[UGI domain]-[NLS]-COOH structure, having optimized nuclear localization signals and wherein the napDNAbp domain comprises a Cas9 nickase, such as an SpCas9n. This BE4max structure was reported to have optimized codon usage for expression in human cells, as reported in Koblan et al., Nat Biotechnol. 2018; 36(9):843-846, herein incorporated by reference.

In other embodiments, exemplary TadCBEs may have a structure that comprises a modified BE4max architecture that contains a napDNAbp domain comprising a Cas9 variant other than Cas9 nickase, such as SpCas9-NG, SaCas9n, eNme2Cas9n, CjCas9n, or enCjCas9n. Accordingly, exemplary TadCBEs of the disclosure may comprise the structure: NH₂-[TadA-CD domain]-[SaCas9n]-[UGI domain]-[UGI domain]-COOH; NH₂-[TadA-CD domain]-[eNme2Cas9n]-[UGI domain]-[UGI domain]-COOH; NH₂-[TadA-CD domain]-[eNme2-C Cas9n]-[UGI domain]-[UGI domain]-COOH; NH₂-[TadA-CD domain]-[CjCas9n]-[UGI domain]-[UGI domain]-COOH; or NH₂-[TadA-CD domain]-[SpCas9-NG]-[UGI domain]-[UGI domain]-COOH, wherein each instance of “]-[” indicates the presence of an optional linker sequence. Additional exemplary TadCBEs may comprise the structure: NH₂-[NLS]-[TadA-CD domain]-[SaCas9n]-[UGI domain]-[UGI domain]-[NLS]-COOH; NH₂-[NLS]-[TadA-CD domain]-[eNme2Cas9n]-[UGI domain]-[UGI domain]-[NLS]-COOH; NH₂-[NLS]-[TadA-CD domain]-[eNme2-C Cas9n]-[UGI domain]-[UGI domain]-[NLS]-COOH; NH₂-[NLS]-[TadA-CD domain]-[CjCas9n]-[UGI domain]-[UGI domain]-[NLS]-COOH; or NH₂-[NLS]-[TadA-CD domain]-[SpCas9-NG]-[UGI domain]-[UGI domain]-[NLS]-COOH.

In some embodiments, the napDNAbp domain comprises an amino acid sequence that has at least 80%, 85%, 90%, 92.5%, 95%, 98%, or 99.5% sequence identity with SEQ ID NO: 19. In some embodiments, the napDNAbp domain comprises the amino acid sequence of SEQ ID NO: 19. In some embodiments, the napDNAbp domain comprises an amino acid sequence that has at least 80%, 85%, 90%, 92.5%, 95%, 98%, or 99.5% sequence identity with SEQ ID NO: 25 or 28. In some embodiments, the napDNAbp domain comprises the amino acid sequence of SEQ ID NO: 25 or 28. In some embodiments, the napDNAbp domain comprises an amino acid sequence that has at least 80%, 85%, 90%, 92.5%, 95%, 98%, or 99.5% sequence identity with any one of SEQ ID NOs: 21 or 22. In some embodiments, the napDNAbp domain comprises the amino acid sequence of SEQ ID NO: 21 or 22.

The disclosed TadCBEs may recognize an expanded PAM sequence, have improved efficiency of deaminating 5′-TC targets, and/or make edits in a narrower target window. In some embodiments, the disclosed cytosine base editors comprise evolved nucleic acid programmable DNA binding proteins (napDNAbp), such as an evolved Cas9.

Exemplary cytosine base editors comprise sequences that are at least 85%, at least 90%, at least 95%, at least 98%, at least 99%, or at least 99.5% identical to the following amino acid sequences, SEQ ID NOs: 19-31. In some embodiments, any of the adenine base editors described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of any of SEQ ID NOs: 19-31. These differences may comprise amino acids that have been inserted, deleted, or substituted relative to the reference sequence. In some embodiments, the disclosed adenosine deaminase domains contain stretches of about 50, about 75, about 100, about 125, about 150, about 175, about 200, about 300, about 400, about 500, or more than 500 consecutive amino acids in common with either of SEQ ID NOs: 19-31.

In some embodiments, the TadCBEs described herein comprise any suitable cytidine deaminase domain known to one of skill in the art, such as those listed in Tables 10 and 11. In some embodiments, the TadCBEs described herein comprise any suitable napDNAbp domain known to one of skill in the art, such as those disclosed herein and elsewhere. The skilled artisan will understand that the examples provided herein are meant for exemplary purposes only and are not intended to be limiting in any way. For instance, the skilled artisan will understand that the methods and compositions disclosed herein may be used to produce TadCBEs comprising various combinations of CDs (e.g., such as those shown in Tables 10 and 11) and napDNAbps (e.g., SpCas9, SaCas9, eNme2-C Cas9, CjCas, etc.).

Exemplary TadCBEs are provided below. The Tad-CD domain is bolded and underlined. In some embodiments, any one of the TadCBEs shown below may further comprises a V106W mutation.

Amino acid sequence of the TadA-CDa base editor (SpCas9n napDNAbp domain)
(TadCBEa)
(SEQ ID NO: 19)
MKRTADGSEFESPKKKRKVSSEVEFSHEYWMRHALTLAKRARDEGAGPVGAV
LVLNNRVIGEGWNRAIGLHDPTAHAEIMALRQGGLVMQNYRLFDATLYVTFEP
CVMCAGAMINSRIGRVVFGVRNSKRGAAGSLMNVLNYPGMNHRVEITEGILAD
ECAALLCDFYRMPRQVENSQKKAQSSINSGGSSGGSSGSETPGTSESATPESSGGSSG
GSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDb base editor (SpCas9n napDNAbp
domain) (TadCBEb)
(SEQ ID NO: 20)
MKRTADGSEFESPKKKRKVSSEVEFSHEYWMRHALTLAKRARDEGAGPVGAV
LVLNNRVIGEGWNRAIGLHDPTAHAEIMALRQGGLVMQNYRLFDATLYVTFEP
CVMCAGAMINSRIGRVVFGVRNSKRGAAGSLMNVLNYPGMNHRVEITEGILAD
ECAALLCDFYRMPRRVFNSQKKAQSSINSGGSSGGSSGSETPGTSESATPESSGGSSG
GSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDc base editor (SpCas9n napDNAbp
domain) (TadCBEc)
(SEQ ID NO: 21)
MKRTADGSEFESPKKKRKVSSEVEFSHEYWMRHALTLAKRARDEGAGPVGAV
LVLNNRVIGEGWNRAIGLHDPTAHAEIMALRQGGLVMQNYRLFDATLYVTFEP
CVMCAGAMINSRIGRVVFGVRNSKRGAAGSLMNVLNYPGMNHRVEITEGILAD
ECAALLCDFYRIPRQVENSQKKAQSSINSGGSSGGSSGSETPGTSESATPESSGGSSGG
SDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFL=VEEDKKHER
HPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDd base editor (SpCas9n napDNAbp
domain) (TadCBEd)
(SEQ ID NO: 22)
MKRTADGSEFESPKKKRKVSSEVEFSHEYWMRHALTLAKRARDERKAPVGAV
LVLNNRVIGEGWNRAIGLHDPTAHAEIIALRQGGLVMQNYRLIDATLYVTFEPC
VMCAGAMINSRIGRVVFGVRNSKRGAAGSLMNVLNYPGMNHRVEITEGILADE
CAALLCDFYRMPRQVENAQKKAQSSINSGGSSGGSSGSETPGTSESATPESSGGSSGG
SDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDe base editor (SpCas9n napDNAbp
domain) (TadCBEe)
(SEQ ID NO: 23)
MKRTADGSEFESPKKKRKVSSEVEFSHEYWMRHALTLAKRARDERAGPVGAV
LVLNNRVIGEGWNRAIGLHDPTAHAEIMALRQGGLVMQNHRLIDATLYVTFEP
CVMCAGAMINSRIGRVVFGVRNSKRGAAGSLMNVLNYPGMNHRVEITEGILAD
ECAALLCDFYRMPRHVFNSQKKAQSSINSGGSSGGSSGSETPGTSESATPESSGGSSG
GSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDa(V106W) base editor (SpCas9n
napDNAbp domain) (TadCBEa(V106W))
(SEQ ID NO: 24)
MKRTADGSEFESPKKKRKVSSEVEFSHEYWMRHALTLAKRARDEGAGPVGAV
LVLNNRVIGEGWNRAIGLHDPTAHAEIMALRQGGLVMQNYRLFDATLYVTFEP
CVMCAGAMINSRIGRVVFGWRNSKRGAAGSLMNVLNYPGMNHRVEITEGILAD
ECAALLCDFYRMPRQVENSQKKAQSSINSGGSSGGSSGSETPGTSESATPESSGGSSG
GSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDd(V106W) base editor (SpCas9n
napDNAbp domain) (TadCBEd(V106W))
(SEQ ID NO: 25)
MKRTADGSEFESPKKKRKVSSEVEFSHEYWMRHALTLAKRARDERKAPVGAV
LVLNNRVIGEGWNRAIGLHDPTAHAEIIALRQGGLVMQNYRLIDATLYVTFEPC
VMCAGAMINSRIGRVVFGWRNSKRGAAGSLMNVLNYPGMNHRVEITEGILADE
CAALLCDFYRMPRQVENAQKKAQSSINSGGSSGGSSGSETPGTSESATPESSGGSSGG
SDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDf base editor (SpCas9n napDNAbp
domain) (TadCBEf)
(SEQ ID NO: 26)
MKRTADGSEFESPKKKRKVSSEVEFSHEYWMRHALTLAKRARDEGEAPVGAV
LVLNNRVIGEGWNRRIGLHDPTAHAEIMALRQGGLVMQNSRLIDATLYVTFEP
CVMCAGAMINSRIGRVVFGVRNSKRGAAGSLMNVLNYPGMNHRVEITEGILAD
ECAALLCDFYRMPRQVENAQKKAQSSINSGGSSGGSSGSETPGTSESATPESSGGSSG
GSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDg base editor (SpCas9n napDNAbp
domain) (TadCBEg)
(SEQ ID NO: 27)
MKRTADGSEFESPKKKRKVSSEVEFSHEYWMRHALTLAKRARDEGEGPVGAV
LVLNNRVIGEGWNRRIGLHDPTAHAEIMALRQGGLVMQNCKLIDATLYVTFEP
CVMCAGAMIHSRIGRVVFGVRNSKRGAAGSLMNVLNYPGMNHRVEITEGILAD
ECAALLCDFYRMPRQVENAQKKAQSSINSGGSSGGSSGSETPGTSESATPESSGGSSG
GSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSKRTADGSEFEPKKKRKV*
Amino acid sequence of the TadA-CDa:eNme2Cas9 base editor
(SEQ ID NO: 28)
MKRTADGSEFESPKKKRKVSSEVEFSHEYWMRHALTLAKRARDEGAGPVGAVLVL
NNRVIGEGWNRAIGLHDPTAHAEIMALRQGGLVMQNYRLFDATLYVTFEPCVMCA
GAMINSRIGRVVFGVRNSKRGAAGSLMNVLNYPGMNHRVEITEGILADECAALLCD
FYRMPRQVFNSQKKAQSSINSGGSSGGSSGSETPGTSESATPESSGGSSGGSAAFKPN
PINYILGLAIGIASVGWAMVEIDEEGNPIRLIDLGVRVFERAEVPKTGDSLAMARRLA
RSVRRLTRRRAHRLLRARRLLKREGVLQAADFDENGLITSLPNTPWQLRAAALDRK
LTPLEWSAVLLHLIKHRGYLSQRKNEGETAAKELGALLKGVANNAHALQTGDFRTP
AELALNKFEKESGHIRNQRGDYSHTFSRKDLQAELILLFEKQKEFGNPHVSGGLKEGI
ETLLMTQRPALSGDAVQKMLGHCTLEPTEPKAAKNTYTAERFIWLTKLNNLRILEQG
SERPLTDTERSTLMDEPYRKSKLTYAQARKLLGLEDTAFFKGLRYGKDNAEASTLM
EMKAYHAISRALEKEGLKDKKSPLNLSSELQDEIGTAFSLFKTDEDITGRLKDRVQPE
ILEALLKHISFDKFVQISLKALRRIVPLMEQGKRYDEACAEIYGVHYGKKNTEEKIYL
PPIPADEIRNPVVLRALSQARKVINGVVRRYGSPARIHIETAREVGKSFKDRKEIAKRQ
EENRKDREKAAAKFREYFPNFVGEPKSKDILKLRLYEQQHGKCLYSGKEINLVRLNE
KGYVEIDHALPFSRTWDDSFNNKVLVLGSENQNKGNQTPYEYFNGKDNSREWQEFK
ARVETSRFPSSKKQRILLQKFDEDGFKECNLNDTRYVNRFLCQFVADHILLTGKGKR
RVVASNGQITNLLRGFWRLRKVRAENDRHHALDAVVVACSTVAMQQKITRFVRYK
EMNAFDGKTVDKETGKVLYQKTHFPQPWEFFAQEVMIRVFGKPDGKPEFEEADTPE
KLRTLLAEKLSSRPEAVHEYVTPLFVSRAPNRKMSGAHKDTLRSAKRFVKHNEKISV
KRVWLTEIKLADLENMVNYKNGREIELYEALKARLEAYGGNAKQAFDPKDNPFYK
KGGQLVKAVRVEKTQKSGVLLNKKNAYTIADNGDMVRVDVFCKVDKKGKNQYFI
VPIYAWQVAENILPDIDCKGYRIDDSYTFCFSLHKYDLIAFQKDEKSKVEFAYYINCD
SSSGGFYLAWHDKGSREQRFRISTQNLALIQKYQVNELGKEIRPCRLKKRPPVSGGSG
GSGGSTNLSDIIEKETGKQLVIQESILMLPEEVEEVIGNKPESDILVHTAYDESTDENV
MLLTSDAPEYKPWALVIQDSNGENKIKMLSGGSGGSGGSTNLSDIIEKETGKQLVIQE
SILMLPEEVEEVIGNKPESDILVHTAYDESTDENVMLLTSDAPEYKPWALVIQDSNGE
NKIKMLSGGSKRTADGSEFEPKKKRKV
Amino acid sequence of the TadA-CDa:SaCas9 base editor
(SEQ ID NO: 29)
MKRTADGSEFESPKKKRKVSSEVEFSHEYWMRHALTLAKRARDEGAGPVGAVLVL
NNRVIGEGWNRAIGLHDPTAHAEIMALRQGGLVMQNYRLFDATLYVTFEPCVMCA
GAMINSRIGRVVFGVRNSKRGAAGSLMNVLNYPGMNHRVEITEGILADECAALLCD
FYRMPRQVFNSQKKAQSSINSGGSSGGSSGSETPGTSESATPESSGGSSGGSGKRNYIL
GLAIGITSVGYGIIDYETRDVIDAGVRLFKEANVENNEGRRSKRGARRLKRRRRHRIQ
RVKKLLFDYNLLTDHSELSGINPYEARVKGLSQKLSEEEFSAALLHLAKRRGVHNVN
EVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMG
HCTYFPEELRSVKYAYNADLYNALNDLNNLVITRDENEKLEYYEKFQIIENVFKQKK
KPTLKQIAKEILVNEEDIKGYRVTSTGKPEFTNLKVYHDIKDITARKEIIENAELLDQIA
KILTIYQSSEDIQEELTNLNSELTQEEIEQISNLKGYTGTHNLSLKAINLILDELWHTND
NQIAIFNRLKLVPKKVDLSQQKEIPTTLVDDFILSPVVKRSFIQSIKVINAIIKKYGLPND
IIIELAREKNSKDAQKMINEMQKRNRQTNERIEEIIRTTGKENAKYLIEKIKLHDMQEG
KCLYSLEAIPLEDLLNNPFNYEVDHIIPRSVSFDNSFNNKVLVKQEENSKKGNRTPFQ
YLSSSDSKISYETFKKHILNLAKGKGRISKTKKEYLLEERDINRFSVQKDFINRNLVDT
RYATRGLMNLLRSYFRVNNLDVKVKSINGGFTSFLRRKWKFKKERNKGYKHHAED
ALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKL
INKSPEKLLMYHHDPQTYQKLKLIMEQYGDEKNPLYKYYEETGNYLTKYSKKDNGP
VIKKIKYYGNKLNAHLDITDDYPNSRNKVVKLSLKPYRFDVYLDNGVYKFVTVKNL
DVIKKENYYEVNSKCYEEAKKLKKISNQAEFIASFYNNDLIKINGELYRVIGVNNDLL
NRIEVNMIDITYREYLENMNDKRPPRIIKTIASKTQSIKKYSTDILGNLYEVKSKKHPQI
IKKGSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKET
GKQLVIQESILMLPEEVEEVIGNKPESDILVHTAYDESTDENVMLLTSDAPEYKPWAL
VIQDSNGENKIKMLSGGSKRTADGSEFEPKKKRKV
Amino acid sequence of the TadA-CDa:SpCas9-NG base editor
(SEQ ID NO: 30)
MKRTADGSEFESPKKKRKVSSEVEFSHEYWMRHALTLAKRARDEGAGPVGAVLVL
NNRVIGEGWNRAIGLHDPTAHAEIMALRQGGLVMQNYRLFDATLYVTFEPCVMCA
GAMINSRIGRVVFGVRNSKRGAAGSLMNVLNYPGMNHRVEITEGILADECAALLCD
FYRMPRQVFNSQKKAQSSINSGGSSGGSSGSETPGTSESATPESSGGSSGGSDKKYSIG
LAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKT
EVQTGGFSKESIRPKRNSDKLIARKKDWDPKKYGGFVSPTVAYSVLVVAKVEKGKS
KKLKSVKELLGITIMERSSFEKNPIDFLEAKGYKEVKKDLIIKLPKYSLFELENGRKRM
LASARFLQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPRAFKYFDT
TIDRKVYRSTKEVLDATLIHQSITGLYETRIDLSQLGGDSGGSGGSGGSTNLSDIIEKET
GKQLVIQESILMLPEEVEEVIGNKPESDILVHTAYDESTDENVMLLTSDAPEYKPWAL
VIQDSNGENKIKMLSGGSGGSGGSTNLSDIIEKETGKQLVIQESILMLPEEVEEVIGNK
PESDILVHTAYDESTDENVMLLTSDAPEYKPWALVIQDSNGENKIKMLSGGSKRTAD
GSEFEPKKKRKV
Amino acid sequence of the TadA-CDa:enCjCas9 base editor
(SEQ ID NO: 31)
MKRTADGSEFESPKKKRKVSSEVEFSHEYWMRHALTLAKRARDEGAGPVGAVLVL
NNRVIGEGWNRAIGLHDPTAHAEIMALRQGGLVMQNYRLFDATLYVTFEPCVMCA
GAMINSRIGRVVFGVRNSKRGAAGSLMNVLNYPGMNHRVEITEGILADECAALLCD
FYRMPRQVFNSQKKAQSSINSGGSSGGSSGSETPGTSESATPESSGGSSGGSARILAFA
IGISSIGWAFSENDELKDCGVRIFTKVENPKTGESLALPRRLARSARKRYARRKARLN
HLKHLIANEFKLNYEDYQSFDESLAKAYKGSLISPYELRFRALNELLSKQDFARVILHI
AKRRGYDDIKNSDDKEKGAILKAIKQNEEKLANYQSVGEYLYKEYFQKFKENSKEF
TNVRNKKESYERCIAQSFLKDELKLIFKKQREFGFSFSKKFEEEVLSVAFYKRALKDF
SHLVGNCSFFTDEKRAPKNSPLAFMFVALTRIINLLNNLKNTEGILYTKDDLNALLNE
VLKNGTLTYKQTKKLLGLSDDYEFKGEKGTYFIEFKKYKEFIKALGEHNLSQDDLNE
JAKDITLIKDEIKLKKALAKYDLNQNQIDSLSKLEFKDHLNISFKALKLVTPLMLEGK
KYDEACNELNLKVAINEDKKDFLPAFNETYYKDEVTNPVVLRAIKEYRKVLNALLK
KYGKVHKINIELAREVGKNHSQRAKIEKEQNENYKAKKDAELECEKLGLKINSKNIL
KLRLFKEQKEFCAYSGEKIKISDLQDEKMLEIDHIYPYSRSFDDSYMNKVLVFTKQN
QEKLNQTPFEAFGNDSAKWQKIEVLAKNLPTKKQKRILDKNYKDKEQKNFKDRNLN
DTRYIARLVLNYTKDYLDFLPLSDDENTKLNDTQKGSKVHVEAKSGMLTSALRHTW
GFSAKDRNNHLHHAIDAVIIAYANNSIVKAFSDFKKEQESNSAELYAKKISELDYKNK
RKFFEPFSGFRQKVLDKIDEIFVSKPERKKPSGALHEETFRKEEEFYQSYGGKEGVLK
ALELGKIRKVNGKIVKNGDMFRVDIFKHKKTNKFYAVPIYTMDFALKVLPNKAVAR
SKKGEIKDWILMDENYEFCFSLYKDSLILIQTKKMQEPEFVYYNAFTSSTVSLIVSKH
DNKFETLSKNQKILFKNANEKEVIAKSIGIQNLKVFEKYIVSALGEVTKAEFRQREDF
KKSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSKRTADGSEFEPKKKRK
V

### Linkers

The fusion proteins described herein may include one or more linkers. As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a deaminase (e.g., a cytidine deaminase or an adenosine deaminase). In some embodiments, a linker joins a Cas domain and a deaminase variant as provided herein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide, or amino acid-based. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)n (SEQ ID NO: 156), (G)n (SEQ ID NO: 157), (EAAAK)n (SEQ ID NO: 158), (GGS)n (SEQ ID NO: 159), (SGGS)n (SEQ ID NO: 160), (XP)n (SEQ ID NO: 161), or any combination thereof, wherein n is independently an integer between 1 and 30, and wherein X is any amino acid. In some embodiments, the linker comprises the amino acid sequence (GGS)n (SEQ ID NO: 159), wherein n is 1, 3, or 7. In some embodiments, the linker comprises the amino acid sequence SGSETPGTSESATPES (SEQ ID NO: 162). In some embodiments, the linker comprises the amino acid sequence SGSETPGTSESA (SEQ ID NO: 163). In some embodiments, the linker comprises the amino acid sequence SGSETPGTSESATPEGGSGGS (SEQ ID NO: 164). In some embodiments, the linker comprises the amino acid sequence SGGSSGGSSGSETPGTSESATPESSGGSSGGS (SEQ ID NO: 165). In some embodiments, the linker comprises the amino acid sequence SGGSGGSGGS (SEQ ID NO: 166). In some embodiments, the linker comprises the amino acid sequence SGGS (SEQ ID NO: 160). In other embodiments, the linker comprises the amino acid sequence SGGSSGGSSGSETPGTSESATPESAGSYPYDVPDYAGSAAPAAKKKKLDGSGSGGSS GGS (SEQ ID NO: 167). In some embodiments, the linker comprises the amino acid sequence GGS (SEQ ID NO: 159), GGSGGS (SEQ ID NO: 168), GGSGGSGGS (SEQ ID NO: 169), SGGSSGGSSGSETPGTSESATPESSGGSSGGSS (SEQ ID NO: 170), SGSETPGTSESATPES (SEQ ID NO: 162), or SGGSSGGSSGSETPGTSESATPESAGSYPYDVPDYAGSAAPAAKKKKLDGSGSGGSS GGS (SEQ ID NO: 167).

In certain embodiments, linkers may be used to link any of the peptides or peptide domains or moieties of the invention (e.g., a napDNAbp linked or fused to a deaminase domain). Any of the domains of the fusion proteins described herein may also be connected to one another through any of the presently described linkers.

### UGI Domains and Other Base Editor Components

In some aspects, the fusion proteins (e.g., base editors) described herein may comprise one or more uracil glycosylase inhibitor (UGI) domains. In some embodiments, the fusion proteins comprise two UGI domains. The UGI domain refers to a protein that is capable of inhibiting a uracil-DNA glycosylase base-excision repair enzyme.

In some embodiments, a UGI domain comprises a wild-type UGI or a UGI as set forth in SEQ ID NO: 272, or a variant thereof. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 272. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 272. In some embodiments, a UGI comprises an amino acid sequence homologous to the amino acid sequence set forth in SEQ ID NO: 272, or an amino acid sequence homologous to a fragment of the amino acid sequence set forth in SEQ ID NO: 272. In some embodiments, proteins comprising UGI, fragments of UGI, or homologs of UGI are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 272. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 272. In some embodiments, the UGI comprises the following amino acid sequence:

>sp|P14739|UNGI_BPPB2 Uracil-DNA glycosylase
inhibitor
(SEQ ID NO: 272)
MTNLSDIIEKETGKQLVIQESILMLPEEVEEVIGNKPESDILVHTAYDES
TDENVMLLTSDAPEYKPWALVIQDSNGENKIKML.

The fusion proteins (e.g., base editors) described herein also may include one or more additional elements. In certain embodiments, an additional element may comprise an effector of base repair, such as an inhibitor of base repair.

In some embodiments, the base editors described herein may comprise one or more heterologous protein domains (e.g., about, or more than about, 1, 2, 3, 4, 5, 6, 7, 8, 9, 10, or more domains in addition to the base editor components). A base editor may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags.

Examples of protein domains that may be fused to a base editor or component thereof (e.g., the napDNAbp domain, the cytidine deaminase domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in U.S. Patent Publication No. 2011/0059502, published Mar. 10, 2011, and incorporated herein by reference in its entirety.

The reporter gene sequences that may be used with the base editors, methods and systems disclosed herein include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), HSV thymidine kinase, rpoB, may be introduced into a cell to encode a gene into which a mutation may be introduced that will confer resistance to a particular medium in a growth selection assay for the described system.

Other exemplary features that may be present are tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein may comprise one or more His tags.

### Nuclear Localization Sequences (NLS)

In various embodiments, the Cas proteins described herein may be fused to one or more nuclear localization sequences (NLS), which help promote translocation of a protein into the cell nucleus. In some embodiments, the fusion proteins described herein may comprise one or more NLS. Such sequences are well-known in the art and can include the following examples:

| Description | Sequence | SEQ ID NO: |
|---|---|---|
| NLS of SV40 large T-Ag | PKKKRKV | 142 |
| NLS | MKRTADGSEFESPKKKRKV | 143 |
| NLS | MDSLLMNRRKFLYQFKNVRWAKG | 144 |
| | RRETYLC | |
| NLS of nucleoplasmin | AVKRPAATKKAGQAKKKKLD | 145 |
| NLS of EGL-13 | MSRRRKANPTKLSENAKKLAKEV | 146 |
| | EN | |
| NLS of c-MYC | PAAKRVKLD | 147 |
| NLS of TUS-protein | KLKIKRPVK | 148 |
| NLS of polyoma large T- | VSRKRPRP | 149 |
| Ag | | |
| NLS of Hepatitis D virus | EGAPPAKRAR | 150 |
| antigen | | |
| NLS of murine p53 | PPQPKKKPLDGE | 151 |
| NLS of PE1 and PE2 | SGGSKRTADGSEFEPKKKRKV | 152 |
| Bipartite sv40 nls | KRTADGSEFESPKKKRKV | 153 |

The NLS examples above are non-limiting. The fusion proteins provided herein may comprise any known NLS sequence, including any of those described in Cokol et al., “Finding nuclear localization signals,” EMBO Rep., 2000, 1(5): 411-415; and Freitas et al., “Mechanisms and Signals for the Nuclear Import of Proteins,” Current Genomics, 2009, 10(8): 550-7, each of which are incorporated herein by reference.

In various embodiments, the fusion proteins and constructs encoding the fusion proteins disclosed herein further comprise one or more, preferably at least two, nuclear localization sequences. In certain embodiments, the fusion proteins comprise at least two NLSs. In embodiments with at least two NLSs, the NLSs can be the same NLSs, or they can be different NLSs. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a fusion protein (e.g., inserted between the encoded napDNAbp component (e.g., any of the Cas14a1 variants disclosed herein) and a deaminase domain (e.g., an adenosine or cytidine deaminase).

The NLSs may be any known NLS sequence in the art. The NLSs may also be any future-discovered NLSs for nuclear localization. The NLSs also may be any naturally-occurring NLS, or any non-naturally-occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 142), MDSLLMNRRKFLYQFKNVRWAKGRRETYLC (SEQ ID NO: 144), KRTADGSEFESPKKKRKV (SEQ ID NO: 153), or KRTADGSEFEPKKKRKV (SEQ ID NO: 155). In other embodiments, NLS comprises the amino acid sequences

(SEQ ID NO: 204)
NLSKRPAAIKKAGQAKKKK,
(SEQ ID NO: 147)
PAAKRVKLD,
(SEQ ID NO: 205)
RQRRNELKRSF,
or
(SEQ ID NO: 206)
NQSSNFGPMKGGNFGGRSSGPYGGGGQYFAKPRNQGGY,
(SEQ ID NO: 276)
KRPAATKKAGQAKKKK,
(SEQ ID NO: 277)
KKTELQTTNAENKTKKL,
(SEQ ID NO: 278)
KRGINDRNFWRGENGRKTR,
or
(SEQ ID NO: 279)
RKSGKIAAIVVKRPRK.

In one aspect of the disclosure, a base editor, prime editor, or other fusion protein may be modified with one or more nuclear localization sequences (NLS), preferably at least two NLSs. In certain embodiments, the fusion proteins are modified with two or more NLSs. The disclosure contemplates the use of any nuclear localization sequence known in the art at the time of the disclosure, or any nuclear localization sequence that is identified or otherwise made available in the state of the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 142)); (ii) a bipartite motif consisting of two basic domains separated by a variable number of spacer amino acids and exemplified by the Xenopus nucleoplasmin NLS (KRXXXXXXXXXXKKKL (SEQ ID NO: 154)); and (iii) noncanonical sequences such as M9 of the hnRNP Al protein, the influenza virus nucleoprotein NLS, and the yeast Gal4 protein NLS (Dingwall and Laskey 1991).

Nuclear localization sequences appear at various points in the amino acid sequences of proteins. NLS have been identified at the N-terminus, the C-terminus, and in the central region of proteins. Thus, the disclosure provides fusion proteins that may be modified with one or more NLSs at the C-terminus and/or the N-terminus, as well as at internal regions of the fusion protein. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein to include one or more NLSs. In one aspect, the fusion proteins may be engineered to express a fusion protein that is translationally fused at its N-terminus or its C-terminus (or both) to one or more NLSs, i.e., to form a Cas protein-NLS fusion construct, base editor-NLS fusion construct, or prime editor-NLS fusion construct. In other embodiments, a fusion protein-encoding nucleotide sequence may be genetically modified to incorporate a reading frame that encodes one or more NLSs in an internal region of the encoded base editor. In addition, the NLSs may include various amino acid linkers or spacer regions encoded between the fusion protein and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a base editor or prime editor and one or more NLSs, among other components.

The fusion proteins described herein may also comprise nuclear localization sequences that are linked to the fusion protein through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the fusion protein by any suitable strategy that effectuates forming a bond (e.g., covalent linkage, hydrogen bonding) between the fusion protein and the one or more NLSs.

### Reduced Off-Target Effects

The skilled artisan will understand that off-target editing events may occur during the editing processes. For example, base editing may result in undesired RNA editing and/or off-target DNA editing of cytidine and/or adenine bases, as a well as insertions and deletions (indels). In some embodiments, the base editors of the present disclosure comprising an evolved cytidine deaminase fused to a napDNAbp, reduces the overall off-target editing frequency to about 0.35% or less.

### Reduced RNA Editing Effects

The evolved base editors disclosed herein have reduced and/or low RNA editing effects. In some embodiments, the base editors are evolved or engineered to have reduced RNA editing effects. The term “RNA editing effects,” as used herein, refers to the introduction of modifications (e.g. deamination) of nucleotides within cellular RNA, e.g., messenger RNA (mRNA). An important goal of DNA base editing efficiency is the modification (e.g. deamination) of a specific nucleotide within DNA, without introducing modifications of similar nucleotides within RNA. RNA editing effects are “low” or “reduced” when a detected mutation is introduced into RNA molecules at a frequency of 0.3% or less.

The present disclosure further provides methods of administering the disclosed TadA-CD base editors wherein the method yields reduced and/or low RNA editing effects. The present disclosure further provides adenine base editors that induce (or yield, provide or cause) reduced and/or low RNA editing effects. In some embodiments, the base editors provide an average cytidine (C) to thymine (T) (C-to-T) editing frequency in cellular mRNA transcripts of 0.3% or less. In some embodiments, the base editors provide an average cytidine (A) to thymine (T) (C-to-T) actual and/or consistent editing frequencies in RNA of about 0.3% or less. The base editors may provide actual or average C-to-T editing frequencies in RNA of about 0.5% or less, 0.4% or less, 0.35% or less, 0.25% or less, 0.2% or less, 0.15% or less, 0.13% or less, 0.1% or less, 0.08% or less, or 0.075% or less. In particular embodiments, the base editors provide an average C-to-T editing frequency of about 0.25%. In particular embodiments, the base editors TadA-CDa, TadA-CDb, and TadA-CDc induces an average C-to-T editing frequency of less than or equal to 0.1% (limit of detection). Other base editor variants, e.g., TadA-CDd and TadA-CDe may, in some embodiments, induce an average C-to-T editing frequency of about 0.3% and 0.2%, respectively. Regardless of the variant type, incorporating a V106W substitution, reduces the off-target RNA editing of all TadA-CD variants to less than or equal to 0.13%.

In some embodiments, the methods induce (or provide, or cause) an average cytidine (C) to thymine (T) editing frequency across the mRNA transcriptome of a human cell (e.g. an HEK293 cell) of about 0.3% or less. The methods may induce actual or average C-to-T transcriptome-wide editing frequencies in RNA of about 0.5% or less, 0.4% or less, 0.35% or less, 0.25% or less, 0.2% or less, 0.15% or less, 0.13% or less, 0.1% or less, 0.08% or less, or 0.075% or less. In particular embodiments, the disclosed methods (such as those involving editing using a TadCBEd or a TadCBEe editor) induce a human mRNA transcriptome-wide average C-to-T editing frequency of 0.3% or 0.2%.

### Reduced Off-Target DNA Editing Effects

Guide RNA-dependent off-target base editing has been reduced through strategies including installation of mutations that increase DNA specificity into the Cas9 component of base editors, adding 5′ guanosine nucleotides to the sgRNA, or delivery of the base editor as a ribonucleoprotein complex (RNP). Guide RNA-independent off-target editing can arise from binding of the deaminase domain of a base editor to C or A bases in a Cas9-independent manner. The Examples below establish that the evolved TadA-CD variants disclosed herein do in fact exhibit detectable guide RNA-independent off-target DNA mutations. However, some evolved TadA-CD variants provided herein, such as TadA-CDa (V106W) through TadACDe (V106W), exhibit reduced Cas9-independent off-target DNA mutations relative to TadA-CDa through TadACDe. The off-target effects of the disclosed cytosine base editors may be measured using an orthogonal R-loop assay, as disclosed in and International Application No. PCT/US2020/624628, filed Nov. 25, 2020, incorporated herein by reference.

In some aspects, provided herein are cytosine base editors and methods of editing DNA by contacting DNA with any of these disclosed base editors that generate (or cause) reduced off-target effects. In various embodiments, methods are designed for determining the off-target editing frequencies of napDNAbp domain-independent (e.g., Cas9-independent) (or gRNA-independent) off-target editing events. Editing events may comprise deamination events of a TadCBE. Off-target deamination events that are dependent on the napDNAbp-guide RNA complex tend to be in sequences that have high sequence identity (e.g., greater than 60% sequence identity) to the target sequence. These types of events arise because of imperfect hybridization of the napDNAbp-guide RNA complex to sequences that share identity with the target sequence. In contrast, off-target events that occur independently of the napDNAbp-guide RNA complex arise as a result of stochastic binding of the base editor to DNA sequences (often sequences that do not share high sequence identity with the target sequence) due to an intrinsic affinity of the base editor of the nucleotide modification domain (e.g., the deaminase domain) of the base editor with DNA. NapDNAbp-independent (e.g., Cas9-independent) editing events may arise, in particular, when the base editor is overexpressed in the system under evaluation, such as a cell or a subject.

In some embodiments, the disclosed TadCBEs exhibit off-target editing frequencies (e.g., A>G editing). In some cases, the position of the adenine within the editing window may effect off-target editing frequencies. In some instances, placement of the adenine in the center of the editing window increases off-target editing frequencies. For example, editing of the PDCD1 target site in HEK293T cells resulted in 34% or 36% adenine base editing for TadA-CDb and Tad-CDc, respectively, and up to 11% for TadCD-d.

In some embodiments, including a V106W mutation within the base editors disclosed herein improves (e.g., lowers) off-target editing frequencies. The addition of V106W to TadA-CDs reduces the A>G editing to a maximum of 12% for TadA-CDb (V106W) and a maximum of 5% for TadA-CDd (V106W) (both maxima observed at PDCD1).

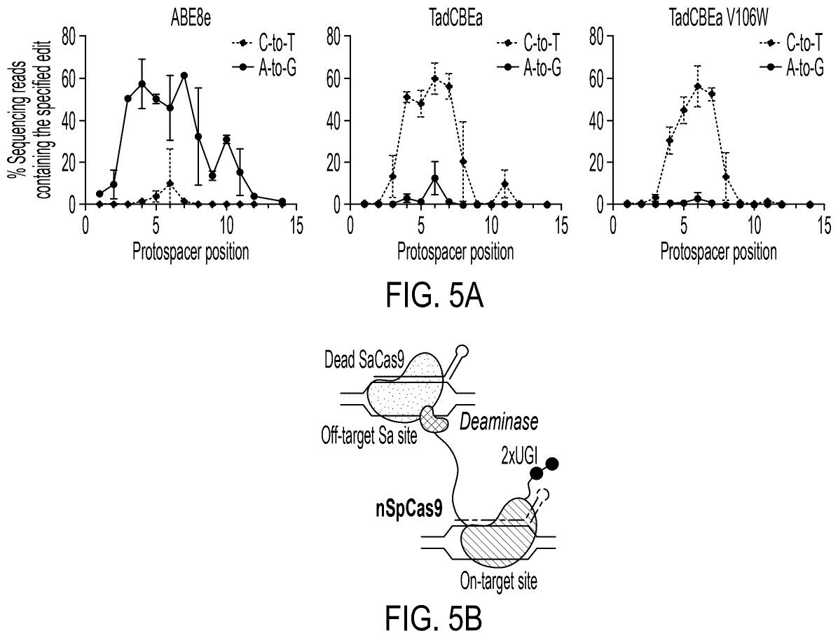

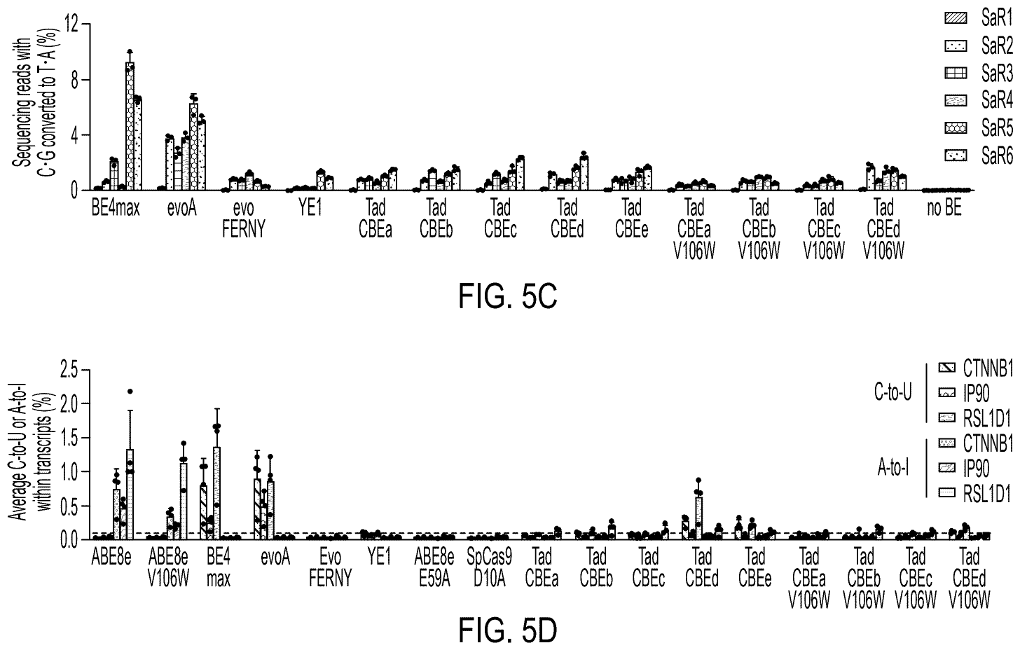

The disclosed TadCBEs exhibit low off-target editing frequencies, and in particular low Cas9-independent off-target editing frequencies, while exhibiting high on-target editing efficiencies. As an example, the TadA-CDa (V106W) based variant may exhibit mean off-target editing frequencies of 0.38% or less while maintaining on-target editing efficiencies of about 80% or more, in target sequences in mammalian cells. As another non-limiting embodiment, TadA-CDb (V106W) based variants may exhibit mean off target editing frequencies of about 0.62% or less while maintaining on-target editing efficiencies of about 80% or more, in target sequences in mammalian cells. Other exemplary embodiments may include variants TadA-CDc, TadA-CDd, and TadA-CDe which may exhibit mean off-targeting editing frequencies of 0.48% or less, 1.1% or less, and 0.05% or less, respectively, while maintaining on-target editing efficiencies of about 80% or more, in target sequences in mammalian cells. In other embodiments, the TadA-CD-V106W-based variants (e.g., TadA-CDa-e) may exhibit indel frequencies of 0.68% or less and/or average off-target editing frequencies of 5% or less, while maintaining on-target editing efficiencies of 80% in target sequences in human cells. (See FIG. 5.) These off-target editing frequencies may be lower than those of several existing cytidine deaminases, such as rAPOBEC1, evoAPOBEC1 (evoA), evoFERNY, and YE1. The Cas-dependent off-target editing exhibited by any of the disclosed TadCBEs may be similar to the levels exhibited for BE4max and EvoA-BE4max.

In some embodiments, the selectivity for cytosine versus adenine deamination for TadA-CDs averaged across greater than 10,000 target sites range from a low of 11-fold favoring cytosine deamination (e.g., for TadA-CDb) to a high of 27-fold (e.g., for TadA-CDd). This selectivity was further enhanced for the V106W variants, from a low of 20-fold (e.g., for TadA-CDb (V106W)) to a high of 48-fold (e.g., for TadA-CDd (V106W)). These over 10,000 target genomic sites may be located in mouse embryonic stem cells, or human embryonic stem cells.

In some embodiments, the disclosed cytidine deaminases exhibit low off-target editing frequencies, and in particular low Cas9-independent off-target editing frequencies, while exhibiting high on-target editing efficiencies when used a variety of Cas homologs and other napDNAbps. In some embodiments, the TadA-CD deaminase or TadA-CDd (V106W) deaminase may exhibit off-target editing frequencies of 0.32% or less while maintaining on-target editing efficiencies of about 80% or more, in target sequences in mammalian cells, when used with a variety of napDNAbps, such as SpCas9, SaCas9, and SaKKH-Cas9.

In some embodiments, the disclosed base editors cause off-target DNA editing (e.g. off-target deamination) frequencies of less than 1.5% (such as less than 1.25%, less than 1.0%, less than 0.75%, or less than 0.5%). In some cases, the off-target editing frequency is less than 1.5%, 1.25%, less than 1.1%, less than 1%, less than 0.75%, less than 0.5%, less than 0.4%, less than 0.25%, less than 0.2%, less than 0.15%, less than 0.1%, or 0.05% or less.

In some embodiments, the disclosed TadCBEs and editing methods comprising the step of contacting a DNA with any of the disclosed TadCBEs result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In some embodiments, the disclosed editing methods result in an actual or average off-target DNA editing frequency of 0.5%, less than 0.5%, less than 0.4%, less than 0.35%, less than 0.3%, less than 0.25%, less than 0.2%, or less than 0.1%. In a particular embodiment, the methods result in an actual or average off-target DNA editing frequency of about 0.32% to about 1.3% (for instance, methods for evaluating the off-target frequencies of TadCBEs comprising TadA-CD-V106W deaminase). These off-target editing frequencies may be obtained in sequences having any level of sequence identity to the target sequence. As used herein to refer to off-target DNA editing frequencies, the modifier “average” refers to a mean value over all editing events detected at sites other than a given target nucleobase pair (e.g., as detected by high-throughput sequencing).

In some embodiments, the disclosed editing methods further result in an actual or average Cas9-independent off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In other words, the disclosed editing methods further result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less in sequences having 60% or less sequence identity to the target sequence. In some embodiments, the disclosed editing methods result in an actual or average off-target DNA editing frequency 0.5%, less than 0.5%, less than 0.4%, less than 0.35%, less than 0.3%, less than 0.25%, less than 0.2%, or less than 0.1%, in sequences having 60% or less sequence identity to the target sequence. In some embodiments, these editing frequencies are obtained in sequences comprising protospacer sequences having 5, 6, 7, 8, 9, 10, or more than 10 mismatches relative to protospacer sequence of the target sequence. In a particular embodiment, the methods result in an actual or average Cas9-independent off-target DNA editing frequency of 0.4% or less.

In various embodiments, the disclosed editing methods result in a ratio of on-target:off-target editing of about 25:1, 50:1, 65:1, 75:1, 80:1, 85:1, 90:1, 95:1, 100:1, 110:1, 125:1, or more than 125:1. In various embodiments, the disclosed editing methods result in a ratio of on-target:off-target editing of about 150:1, 200:1, 300:1, 400:1, 500:1, 600:1, 700:1, 800:1, 900:1, 1000:1, 1100:1, 1200:1, 1250:1, 1275:1, 1300:1, 1325:1, 1350:1, 1400:1, 1500:1, or more than 1500:1. As used herein, a ratio of on-target:off-target editing is equivalent to a ratio of sequencing reads reflecting on-target deaminations relative to deaminations of known or predicted off-target sites, or candidate off-target sites. Candidate off-target sites may be identified, and hence the ratio of on-target:off-target editing may be measured, using an experimental assay or a computation algorithm (e.g., Cas-OFFinder). For example, candidate off-target sites may be identified using an experimental assay such as EndoV-Seq, GUIDE-Seq, or CIRCLE-Seq.

In some embodiments, the disclosed editing methods result in a ratio of on-target:off-target editing in a CXCR4 or CCR5 gene of about 25:1, 50:1, 65:1, 75:1, 80:1, 85:1, 90:1, 95:1, 100:1, 110:1, 125:1, or more than 125:1. In various embodiments, the disclosed editing methods result in a ratio of on-target:off-target editing in a CXCR4 or CCR5 gene of about 150:1, 200:1, 300:1, 400:1, 500:1, 600:1, 700:1, 800:1, 900:1, 1000:1, 1100:1, 1200:1, 1250:1, 1275:1, 1300:1, 1325:1, 1350:1, 1400:1, 1500:1, or more than 1500:1. In some cases, the ratio of on-target:off-target editing is about 90:1 or more in an CXCR4 or CCR5 gene.

In some embodiments, the disclosed editing methods result in a ratio of on-target:off-target editing that is equivalent to the ratio of intended point mutations:unintended point mutations. In some embodiments, the disclosed editing methods result in a ratio of intended point mutations to unintended point mutations that is at least 25:1, at least 30:1, at least 40:1, at least 50:1, at least 75:1, at least 90:1, at least 100:1, at least 150:1, at least 200:1, at least 250:1, at least 500:1, at least 1000:1, at least 1100:1, at least 1200:1, at least 1250:1, at least 1300:1, at least 1350:1, at least 1400:1, at least 1500:1, or more.

In some embodiments, the disclosed editing methods result in, and the disclosed base editors generate, a very low degree of bystander edits (i.e., synonymous off-target point mutations at nucleobases that are near the target base and do not change the outcome of the intended editing method). In some embodiments, the disclosed editing methods result in less than 10, less than 9, less than 8, less than 7, less than 6, less than 5, less than 4, less than 3, less than 2, less than 1, or zero non-silent bystander edits.

### Reduced Indel Frequencies

Some aspects of the disclosure are based on the recognition that any of the cytosine base editors provided herein are capable of modifying a specific DNA base without generating a significant proportion of indels. An “indel”, as used herein, refers to the insertion or deletion of a nucleotide base within a DNA substrate. Such insertions or deletions can lead to frame shift mutations within a coding region of a gene. In some embodiments, it is desirable to generate cytosine base editors that efficiently modify (e.g. mutate or deaminate) a specific nucleotide within a DNA, without generating a large number of insertions or deletions (i.e., indels) in the nucleic acid (while at the same time having lower RNA editing effects than existing adenine base editors).

In certain embodiments, any of the cytosine base editors provided herein are capable of generating a greater proportion of intended modifications (e.g., point mutations or deaminations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method, for example the methods used in the below Examples. In some embodiments, indel frequencies correspond to the percent of total sequencing reads at a target sequence that contain indels. Accordingly, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the cytosine base editors provided herein are capable of limiting formation of indels in a region of a DNA substrate. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein may induce an indel formation at a region of a nucleic acid at frequencies of less than 1%, less than 1.5%, less than 2%, less than 2.5%, less than 2.8%, less than 3%, less than 3.5%, less than 4%, less than 4.5%, less than 5%, less than 6%, less than 7%, less than 8%, less than 9%, less than 10%, less than 12%, less than 15%, or less than 20%. In some embodiments, any of the base editors provided herein may induce or generate less than 20%, 19%, 18%, 16%, 14%, 12%, 10%, 8%, 6%, 4%, 3%, 2%, 1%, 0.5%, 0.2%, 0.1%, or 0.05% indel formation when contacted with a nucleic acid comprising a target sequence. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a cytosine base editor, such as through transfection a vector encoding the editor. In some embodiments, indel frequency is determined after 3 days.

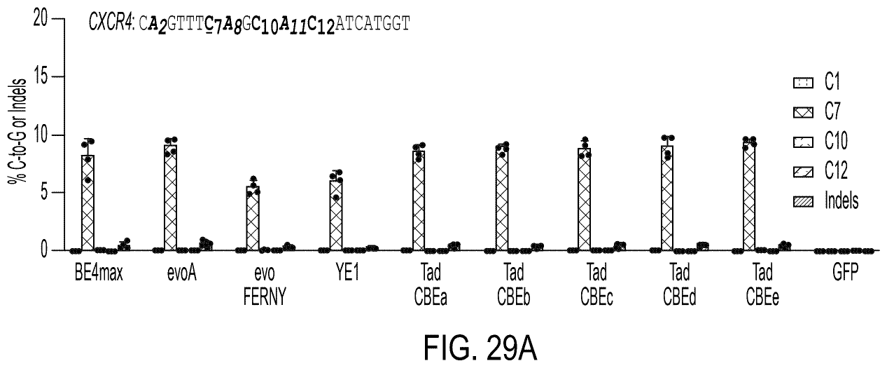

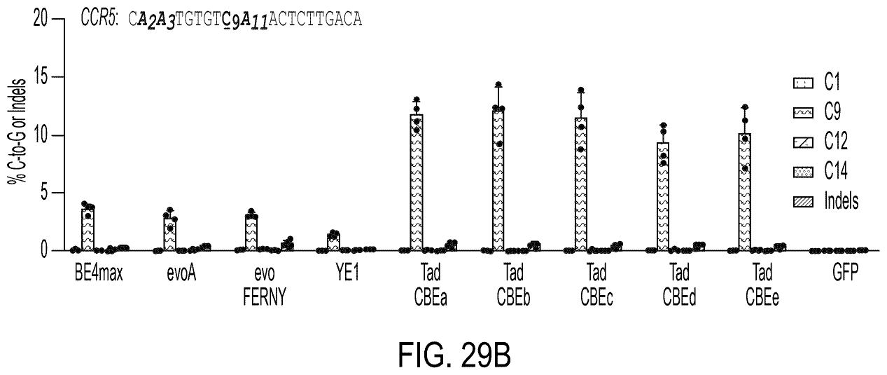

In some embodiments, the TadA-CDa-e base editors may induce an indel formation at a region of a nucleic acid comprising a target sequence at frequencies of less than 1.1% (see FIG. 29A). In some embodiments, the disclosed editing methods that use the disclosed TadCBEs may result in less than 20%, 19%, 18%, 16%, 14%, 12%, 10%, 8%, 6%, 4%, 2%, 1.5%, 1%, 0.5%, 0.2%, or 0.1% indel formation in a nucleic acid (e.g., a DNA) comprising a target sequence.

Some aspects of the disclosure are based on the recognition that any of the base editors provided herein are capable of efficiently generating an intended mutation, such as a point mutation, in DNA (e.g. DNA within a genome of a subject) without generating a significant number of unintended mutations, such as unintended point mutations. In some embodiments, an intended mutation is a mutation that is generated by a specific base editor bound to a gRNA, specifically designed to generate the intended mutation (e.g. deamination). In some embodiments, the intended mutation is a mutation associated with a disease or disorder, such as sickle cell disease or HIV/AIDS. In some embodiments, the intended mutation is an adenine (A) to guanine (G) point mutation associated with a disease or disorder. In some embodiments, the intended mutation is a thymine (T) to cytosine (C) point mutation associated with a disease or disorder. In some embodiments, the intended mutation is an adenine (A) to guanine (G) point mutation within the coding region of a gene. In some embodiments, the intended mutation is a thymine (T) to cytosine (C) point mutation within the coding region of a gene.

In some embodiments, the intended mutation is a deamination that generates a stop codon, for example, a premature stop codon within the coding region of a gene. In some embodiments, the intended mutation is a mutation that eliminates a stop codon. In some embodiments, the intended mutation eliminates a stop codon comprising the nucleic acid sequence 5′-TAG-3′, 5′-TAA-3′, or 5′-TGA-3′.

In some embodiments, the intended mutation is a deamination that alters the regulatory sequence of a gene (e.g., a gene promoter or gene repressor). In some embodiments, the intended mutation is a deamination introduced into the gene promoter. In particular embodiments, the deamination introduced into the gene promoter leads to a decrease in the transcription of a gene operably linked to the gene promoter. In other embodiments, the deamination leads to an increase in the transcription of a gene operably linked to the gene promoter.

In some embodiments, the intended mutation is a deamination that alters the splicing of a gene. Accordingly, in some embodiments, the intended deamination results in the introduction of a splice site in a gene. In other embodiments, the intended deamination results in the removal of a splice site.

In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in this section and the following section of the disclosure may be applied to any of the base editors, or methods of using the base editors provided herein.

### Methods for Generating the Cytosine Base Editors

The invention further relates in various aspects to methods of generating the disclosed cytosine base editors by various modes of manipulation that include, but are not limited to, codon optimization to achieve greater expression levels in a cell, and the use of nuclear localization sequences (NLSs), preferably at least two NLSs, e.g., two bipartite NLSs, to increase the localization of the expressed base editors into a cell nucleus.

The base editors contemplated herein can include modifications that result in increased expression, for example, through codon optimization.

In some embodiments, a polynucleotide encoding any of the disclosed base editors (or a component thereof) is codon optimized for expression in particular cells, such as eukaryotic cells (e.g., human cells). The eukaryotic cells may be those of or derived from a particular organism, such as a mammal, including, but not limited to, human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

The above description is meant to be non-limiting with regard to making base editors having increased expression, and thereby increase editing efficiencies.

### Directed Evolution Methods (e.g., PACE or PANCE)

Various embodiments of the disclosure relate to providing directed evolution methods and systems (e.g., appropriate vectors, cells, phage, flow vessels, etc.) for engineering of the base editors or base editor domains of the present disclosure. The disclosure provides vector systems for the disclosed directed evolution methods to engineer any of the disclosed base editors or base editor domains (e.g., the evolved adenosine deaminase domains of any of the disclosed base editors).

The directed evolution vector systems and methods provided herein allow for a gene of interest (e.g., a base editor- or adenosine deaminase-encoding gene) in a viral vector to be evolved over multiple generations of viral life cycles in a flow of host cells to acquire a desired function or activity.

Some embodiments of this disclosure provide methods of phage-assisted continuous evolution (PACE) comprising (a) contacting a population of bacterial host cells with a population of bacteriophages that comprise a gene of interest to be evolved and that are deficient in a gene required for the generation of infectious phage, wherein (1) the phage allows for expression of the gene of interest in the host cells; (2) the host cells are suitable host cells for phage infection, replication, and packaging; and (3) the host cells comprise an expression construct encoding the gene required for the generation of infectious phage, wherein expression of the gene is dependent on a function of a gene product of the gene of interest. In some embodiments, the method further comprises (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that have not been infected by the phage. In some embodiments, the method further comprises (c) isolating a mutated phage replication product encoding an evolved protein from the population of host cells.

In PACE, the gene under selection is encoded on the M13 bacteriophage genome. Its activity is linked to M13 propagation by controlling expression of gene III so that only active variants produce infectious progeny phage. Phage are continuously propagated and mutagenized, but mutations accumulate only in the phage genome, not the host or its selection circuit, because fresh host cells are continually flowed into (and out of) the growth vessel, effectively resetting the selection background.

### Development of a PANCE/PACE Evolution Circuit

PACE enables the rapid continuous evolution of biomolecules through many generations of mutation, selection, and replication per day. During PACE, host E. coli cells continuously dilute a population of bacteriophage (selection phage, SP) containing the gene of interest. The gene of interest replaces gene III on the SP, which is required for progeny phage infectivity. SP containing desired gene variants trigger host-cell gene III expression from an accessory plasmid (AP). Host-cell DNA plasmids encode a genetic circuit that links the desired activity of the protein encoded in the SP to the expression of gene III on the AP. Thus, SP variants containing desired gene variants can propagate, while phage encoding inactive variants do not generate infectious progeny and are rapidly diluted out of the culture vessel (or lagoon). An arabinose-inducible mutagenesis plasmid (MP) controls the phage mutation rate.

The key to new PACE selections is linking gene III expression to the activity of interest. A low stringency selection was designed in which base editing activates T7 RNA polymerase, which transcribes gIII. A single editing event can lead to high output amplification immediately upon transcription of the edited DNA. Reference is made to International Patent Publication WO 2019/023680, published Jan. 31, 2019; Badran, A. H. & Liu, D. R. In vivo continuous directed evolution. Curr. Opin. Chem. Biol. 24, 1-10 (2015); Dickinson, B. C., Packer, M. S., Badran, A. H. & Liu, D. R. A system for the continuous directed evolution of proteases rapidly reveals drug-resistance mutations. Nat. Commun. 5, 5352 (2014); Hubbard, B. P. et al. Continuous directed evolution of DNA-binding proteins to improve TALEN specificity. Nat. Methods 12, 939-942 (2015); Wang, T., Badran, A. H., Huang, T. P. & Liu, D. R. Continuous directed evolution of proteins with improved soluble expression. Nat. Chem. Biol. 14, 972-980 (2018), and Thuronyi, B. W. et al. Continuous evolution of base editors with expanded target compatibility and improved activity. Nat. Biotechnol., 1070-1079 (2019), each of which is herein incorporated by reference.

Reference is also made a high-throughput mammalian DNA base editor library, generated using the BE-HIVE tool, that used to evaluate the editing activity and editing window of adenine base editors. The BE-HIVE model is described in additional detail in International Application No. PCT/US2021/016924, which published as Publication No. WO/2021/158995 on Aug. 12, 2021

The disclosure provides vector systems for performing directed evolution of adenosine deaminase domains of a cytosine base editor. In some embodiments, the vector systems comprise an expression construct that comprises a nucleic acid encoding a split intein portion (e.g., the N-terminal portion or the C-terminal portion of a split intein) operably linked to a nucleic acid encoding a gene required for the production of infectious phage particles, such as gIII protein (pIII protein), or a portion (e.g., fragment) thereof. In some embodiments, the split intein portion is the C-terminal portion of a split intein (e.g., the C-terminal portion of an Npu (Nostoc punctiforme) split intein). In some embodiments, the split intein C-terminal portion is positioned upstream of (e.g., 5′ relative to) the nucleic acid encoding the gene required for the production of infectious phage particles, or portion thereof. In some embodiments, the split intein portion is the N-terminal portion of a split intein (e.g., the N-terminal portion of an Npu split intein). In some embodiments, the split intein N-terminal portion is positioned downstream of (e.g., 3′ relative to) the nucleic acid encoding the gene required for the production of infectious phage particles, or portion thereof. In some embodiments, the disclosed vector system expression constructs (e.g., in a first accessory plasmid or second accessory plasmid) further comprises a sequence encoding luxAB.

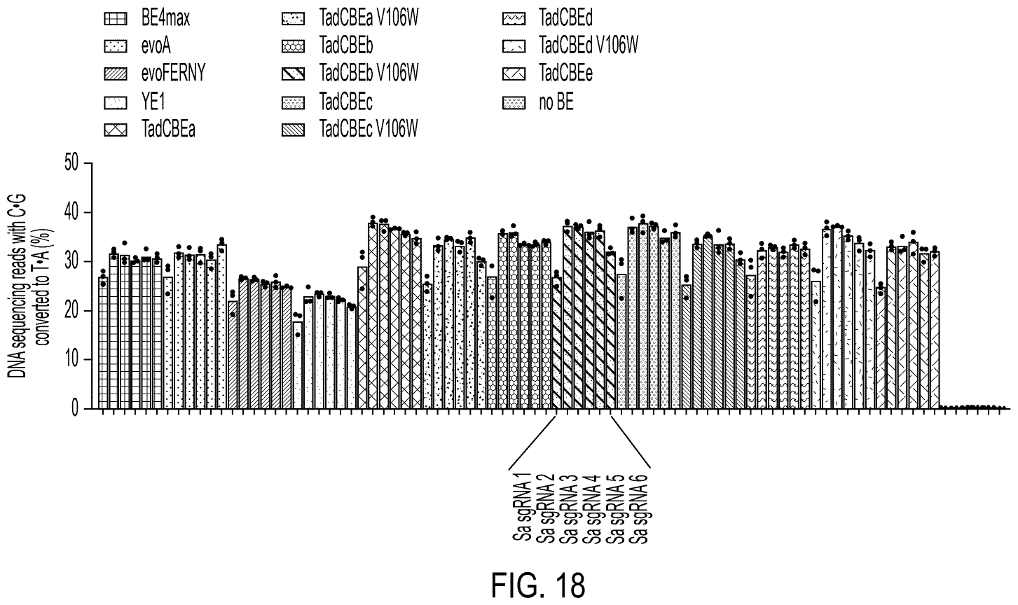

In some embodiments, the vector systems described herein comprising: (i) a selection plasmid comprising an isolated nucleic acid comprising an expression construct encoding an evolved adenosine deaminase comprising, in the following order: an adenosine deaminase protein and a sequence encoding an N-terminal portion of a split intein; (ii) a first accessory plasmid comprising an isolated nucleic acid comprising an expression construct comprising, in the following order: a sequence encoding a guide RNA operably controlled by a Lac promoter and a sequence encoding a M13 phage gIII protein signal peptide operably controlled by a T7 RNA promoter, wherein the sequence encoding the gIII protein signal peptide lacks one or more nucleic acid bases of the signal peptide domain; (iii) a second accessory plasmid comprising an isolated nucleic acid comprising an expression construct comprising, in the following order: a sequence encoding the C-terminal portion of a split intein and a sequence encoding a dCas9; and (iv) a third accessory plasmid comprising an isolated nucleic acid comprising an expression construct comprising, in the following order: a promoter, a ribosome binding site, and a sequence encoding a T7 RNA polymerase (RNAP) comprising mutations that give rise to two stop codons that can be corrected upon adenine base editing (see FIGS. 1B and 18). In some embodiments, the split intein is an Npu split intein. In some embodiments, these stop codons are created at positions 57 and 58. In some embodiments, adenine base editing corrects mutations at positions 57 and 58 in the T7 RNAP coding region and induces substitution back to the wild-type Q57 and R58 (see FIG. 1C). In certain embodiments, the disclosed vector systems further comprise a plurality of third accessory plasmids, each comprising a unique ribosome binding site or a unique promoter. As many as five, six, seven, eight, nine, or ten variants of the third accessory plasmid may be developed with different promoters and ribosome binding sites (RBS) to tune the negative stringency of the PACE evolution. In certain embodiments, the vector systems further comprise a mutagenesis plasmid.

In some embodiments, a vector system is provided as part of a kit, which is useful, in some embodiments, for performing PACE to produce adenosine deaminase protein variants. For example, in some embodiments, a kit comprises a first container housing the selection phagemid of the vector system, a second container housing the first accessory plasmid of the vector system, and a third container housing the second accessory plasmid of the vector system. In some embodiments, a kit further comprises a mutagenesis plasmid. Mutagenesis plasmids for PACE are generally known in the art, and are described, for example in International PCT Application No. PCT/US2016/027795, filed Sep. 16, 2016, published as WO 2016/168631, the entire contents of which are incorporated herein by reference. In some embodiments, the kit further comprises a set of written or electronic instructions for performing PACE.

In some embodiments of the directed evolution methods and systems provided herein, the viral vector or the phage is a filamentous phage, for example, an M13 phage, such as an M13 selection phage as described in more detail elsewhere herein. In some such embodiments, the gene required for the production of infectious viral particles is the M13 gene III (gIII).

In some embodiments, the incubating of the host cells is for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, a viral vector/host cell combination is chosen in which the life cycle of the viral vector is significantly shorter than the average time between cell divisions of the host cell. Average cell division times and viral vector life cycle times are well known in the art for many cell types and vectors, allowing those of skill in the art to ascertain such host cell/vector combinations. In certain embodiments, host cells are being removed from the population of host cells contacted with the viral vector at a rate that results in the average time of a host cell remaining in the host cell population before being removed to be shorter than the average time between cell divisions of the host cells, but to be longer than the average life cycle of the viral vector employed. The result of this is that the host cells, on average, do not have sufficient time to proliferate during their time in the host cell population while the viral vectors do have sufficient time to infect a host cell, replicate in the host cell, and generate new viral particles during the time a host cell remains in the cell population. This assures that the only replicating nucleic acid in the host cell population is the viral vector, and that the host cell genome, the accessory plasmid, or any other nucleic acid constructs cannot acquire mutations allowing for escape from the selective pressure imposed.

For example, in some embodiments, the average time a host cell remains in the host cell population is about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes.

In some embodiments, the average time a host cell remains in the host cell population depends on how fast the host cells divide and how long infection (or conjugation) requires. In general, the flow rate should be faster than the average time required for cell division, but slow enough to allow viral (or conjugative) propagation. The former will vary, for example, with the media type, and can be delayed by adding cell division inhibitor antibiotics (FtsZ inhibitors in E. coli, etc.). Since the limiting step in continuous evolution is production of the protein required for gene transfer from cell to cell, the flow rate at which the vector washes out will depend on the current activity of the gene(s) of interest. In some embodiments, titratable production of the protein required for the generation of infectious particles, as described herein, can mitigate this problem. In some embodiments, an indicator of phage infection allows computer-controlled optimization of the flow rate for the current activity level in real-time.

In some embodiments, the fresh host cells comprise the accessory plasmid required for selection of viral vectors, for example, the accessory plasmid comprising the gene required for the generation of infectious phage particles that is lacking from the phages being evolved. In some embodiments, the host cells are generated by contacting an uninfected host cell with the relevant vectors, for example, the accessory plasmid and, optionally, a mutagenesis plasmid, and growing an amount of host cells sufficient for the replenishment of the host cell population in a continuous evolution experiment. Methods for the introduction of plasmids and other gene constructs into host cells are well known to those of skill in the art and the invention is not limited in this respect. For bacterial host cells, such methods include, but are not limited to, electroporation and heat-shock of competent cells.

In some embodiments, the accessory plasmid comprises a selection marker, for example, an antibiotic resistance marker, and the fresh host cells are grown in the presence of the respective antibiotic to ensure the presence of the plasmid in the host cells. Where multiple plasmids are present, different markers are typically used. Such selection markers and their use in cell culture are known to those of skill in the art, and the invention is not limited in this respect.

In particular embodiments, a first accessory plasmid comprises gene III, and a second accessory plasmid comprises a T7 RNAP gene deactivated by a G to T mutation, which results in an early stop codon. A third accessory plasmid may comprise a nucleotide encoding a dCas9 fused at the N terminus to the C-terminal half of a fast-splicing intein. An exemplary phage plasmid may comprise a nucleotide encoding an adenosine deaminase fused at the C terminus to the N-terminal half of the fast-splicing intein. The full-length base editor is reconstituted from the two intein components.

In some embodiments, the selection marker is a spectinomycin antibiotic resistance marker. In other embodiments, the selection marker is a chloramphenicol or carbenicillin resistance marker. Cells may be transformed with a selection plasmid containing an inactivated spectinomycin resistance gene with a mutation at an active site that requires A:T to C:G editing to correct. Cells that fail to install the correct transversion mutation in the spectinomycin resistance gene will die, while cells that make the correction will survive. E. coli cells expressing an sgRNA targeting the active site mutation in the spectinomycin resistance gene and a nucleotide modification domain-dCas9 base editor are plated onto 2×YT agar with 256 μg/mL of spectinomycin. Surviving colonies (measured through CFUs) were sequenced to find consensus mutations in the base editors expressed in the evolved survivors. A similar selection assay was used to evolve adenosine deaminase activity in DNA during adenine base editor development, as described in Gaudelli, N. M. et al., Programmable base editing of A·T to G·C in genomic DNA without DNA cleavage. Nature 551, 464-471 (2017), incorporated herein in its entirety by reference.

In some embodiments, the host cell population in a continuous evolution experiment is replenished with fresh host cells growing in a parallel, continuous culture. In some embodiments, the cell density of the host cells in the host cell population contacted with the viral vector and the density of the fresh host cell population is substantially the same.

Typically, the cells being removed from the cell population contacted with the viral vector comprise cells that are infected with the viral vector and uninfected cells. In some embodiments, cells are being removed from the cell populations continuously, for example, by effecting a continuous outflow of the cells from the population. In other embodiments, cells are removed semi-continuously or intermittently from the population. In some embodiments, the replenishment of fresh cells will match the mode of removal of cells from the cell population, for example, if cells are continuously removed, fresh cells will be continuously introduced. However, in some embodiments, the modes of replenishment and removal may be mismatched, for example, a cell population may be continuously replenished with fresh cells, and cells may be removed semi-continuously or in batches.

In some embodiments, the rate of fresh host cell replenishment and/or the rate of host cell removal is adjusted based on quantifying the host cells in the cell population. For example, in some embodiments, the turbidity of culture media comprising the host cell population is monitored and, if the turbidity falls below a threshold level, the ratio of host cell inflow to host cell outflow is adjusted to effect an increase in the number of host cells in the population, as manifested by increased cell culture turbidity. In other embodiments, if the turbidity rises above a threshold level, the ratio of host cell inflow to host cell outflow is adjusted to effect a decrease in the number of host cells in the population, as manifested by decreased cell culture turbidity. Maintaining the density of host cells in the host cell population within a specific density range ensures that enough host cells are available as hosts for the evolving viral vector population, and avoids the depletion of nutrients at the cost of viral packaging and the accumulation of cell-originated toxins from overcrowding the culture.

In some embodiments, the cell density in the host cell population and/or the fresh host cell density in the inflow is about 102 cells/ml to about 1012 cells/ml. In some embodiments, the host cell density is about 102 cells/ml, about 103 cells/ml, about 104 cells/ml, about 105 cells/ml, about 5·105 cells/ml, about 106 cells/ml, about 5·106 cells/ml, about 107 cells/ml, about 5·107 cells/ml, about 108 cells/ml, about 5·108 cells/ml, about 109 cells/ml, about 5·109 cells/ml, about 1010 cells/ml, or about 5·1010 cells/ml. In some embodiments, the host cell density is more than about 1010 cells/ml.

In some embodiments, the host cell population is contacted with a mutagen. In some embodiments, the cell population contacted with the viral vector (e.g., the phage), is continuously exposed to the mutagen at a concentration that allows for an increased mutation rate of the gene of interest, but is not significantly toxic for the host cells during their exposure to the mutagen while in the host cell population. In other embodiments, the host cell population is contacted with the mutagen intermittently, creating phases of increased mutagenesis, and accordingly, of increased viral vector diversification. For example, in some embodiments, the host cells are exposed to a concentration of mutagen sufficient to generate an increased rate of mutagenesis in the gene of interest for about 10%, about 20%, about 50%, or about 75% of the time.

In some embodiments, the host cells comprise a mutagenesis expression construct, for example, in the case of bacterial host cells, a mutagenesis plasmid. In some embodiments, the mutagenesis plasmid comprises a gene expression cassette encoding a mutagenesis-promoting gene product, for example, a proofreading-impaired DNA polymerase. In other embodiments, the mutagenesis plasmid, including a gene involved in the SOS stress response, (e.g., UmuC, UmuD′, and/or RecA). In some embodiments, the mutagenesis-promoting gene is under the control of an inducible promoter. Suitable inducible promoters are well known to those of skill in the art and include, for example, arabinose-inducible promoters, tetracycline or doxycyclin-inducible promoters, and tamoxifen-inducible promoters. In some embodiments, the host cell population is contacted with an inducer of the inducible promoter in an amount sufficient to effect an increased rate of mutagenesis. For example, in some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation.

In some embodiments, diversifying the viral vector population is achieved by providing a flow of host cells that does not select for gain-of-function mutations in the gene of interest for replication, mutagenesis, and propagation of the population of viral vectors. In some embodiments, the host cells are host cells that express all genes required for the generation of infectious viral particles, for example, bacterial cells that express a complete helper phage, and, thus, do not impose selective pressure on the gene of interest. In other embodiments, the host cells comprise an accessory plasmid comprising a conditional promoter with a baseline activity sufficient to support viral vector propagation even in the absence of significant gain-of-function mutations of the gene of interest. This can be achieved by using a “leaky” conditional promoter, by using a high-copy number accessory plasmid, thus amplifying baseline leakiness, and/or by using a conditional promoter on which the initial version of the gene of interest effects a low level of activity while a desired gain-of-function mutation effects a significantly higher activity.

Detailed methods of procedures for directing continuous evolution of base editors in a population of host cells using phage particles are disclosed in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, published as WO 2019/241649 on Dec. 19, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Publication No. WO 2020/041751, published on Feb. 27, 2020, each of which are incorporated herein by reference.

Methods and strategies to design conditional promoters suitable for carrying out the selection strategies described herein are well known to those of skill in the art. For an overview over exemplary suitable selection strategies and methods for designing conditional promoters driving the expression of a gene required for cell-cell gene transfer, e.g., gene III (gIII), see Vidal and Legrain, Yeast n-hybrid review, Nucleic Acid Res. 27, 919 (1999), incorporated herein in its entirety.

The disclosure provides vectors for the continuous evolution processes. In some embodiments, phage vectors for phage-assisted continuous evolution are provided. In some embodiments, a selection phage is provided that comprises a phage genome deficient in at least one gene required for the generation of infectious phage particles and a gene of interest to be evolved. Reference is made to International Patent Publication WO 2019/023680, published Jan. 31, 2019, herein incorporated by reference.

For example, in some embodiments, the selection phage comprises an M13 phage genome deficient in a gene required for the generation of infectious M13 phage particles, for example, a full-length gIII. In some embodiments, the selection phage comprises a phage genome providing all other phage functions required for the phage life cycle except the gene required for generation of infectious phage particles. In some such embodiments, an M13 selection phage is provided that comprises a gI, gII, gIV, gV, gVI, gVII, gVIII, gIX, and a gX gene, but not a full-length gIII. In some embodiments, the selection phage comprises a 3′-fragment of gIII, but no full-length gIII. The 3′-end of gIII comprises a promoter and retaining this promoter activity is beneficial, in some embodiments, for an increased expression of gVI, which is immediately downstream of the gIII 3′-promoter, or a more balanced (wild-type phage-like) ratio of expression levels of the phage genes in the host cell, which, in turn, can lead to more efficient phage production. In some embodiments, the 3′-fragment of gIII gene comprises the 3′-gIII promoter sequence. In some embodiments, the 3′-fragment of gIII comprises the last 180 bp, the last 150 bp, the last 125 bp, the last 100 bp, the last 50 bp, or the last 25 bp of gII. In some embodiments, the 3′-fragment of gIII comprises the last 180 bp of gIII.

M13 selection phage is provided that comprises a gene of interest in the phage genome, for example, inserted downstream of the gVIII 3′-terminator and upstream of the gIII-3′-promoter. In some embodiments, an M13 selection phage is provided that comprises a multiple cloning site for cloning a gene of interest into the phage genome, for example, a multiple cloning site (MCS) inserted downstream of the gVIII 3′-terminator and upstream of the gIII-3′-promoter.

Some embodiments of this disclosure provide a vector system for continuous evolution procedures, comprising of a viral vector, for example, a selection phage, and a matching accessory plasmid. In some embodiments, a vector system for phage-based continuous directed evolution is provided that comprises (a) a selection phage comprising a gene of interest to be evolved, wherein the phage genome is deficient in a gene required to generate infectious phage; and (b) an accessory plasmid comprising the gene required to generate infectious phage particle under the control of a conditional promoter, wherein the conditional promoter is activated by a function of a gene product encoded by the gene of interest.

In some embodiments, the selection phage is an M13 phage as described herein. For example, in some embodiments, the selection phage comprises an M13 genome including all genes required for the generation of phage particles, for example, gI, gII, gIV, gV, gVI, gVII, gVIII, gIX, and gX gene, but not a full-length gIII gene. In some embodiments, the selection phage genome comprises an F1 or an M13 origin of replication. In some embodiments, the selection phage genome comprises a 3′-fragment of gIII gene. In some embodiments, the selection phage comprises a multiple cloning site upstream of the gIII 3′-promoter and downstream of the gVIII 3′-terminator.

In an exemplary PACE methodology, host cells each containing a mutagenesis plasmid are diluted into 5 mL Davis Rich Medium (DRM) with appropriate antibiotics and grown to an A600 of 0.4-0.8. Cells are then used to inoculate a chemostat (60 mL), which may be maintained under continuous dilution with fresh DRM at 1-1.5 volumes per hour to keep cell density roughly constant. Lagoons are initially filled with DRM, then continuously diluted with chemostat culture for at least 2 hours before seeding with phage. A stock solution of arabinose (1 M) may be pumped directly into lagoons (10 mM final) for 1 hour before the addition of selection phage (SP). For the first 12 hours after phage inoculation, anhydrotetracycline is present in the stock solution (3.3 μg/mL). Lagoons may be seeded at a starting titer of ˜107 pfu per mL. Dilution rate may be adjusted by modulating lagoon volume (5-20 mL) and/or culture inflow rate (10-20 mL/h). Lagoons may be sampled every 24 hours by removal of culture (500 μL) by syringe. Samples are centrifuged at 13,500 g for 2 minutes and the supernatant removed and stored at 4° C. Titers are evaluated by plaquing. The presence of T7 RNAP or gene III recombinant phage is monitored by plaquing on S2060 cells containing pT7-AP and no plasmid. Phage genotypes may be assessed from single plaques by diagnostic PCR.

Some embodiments of this disclosure provide a method of non-continuous evolution of a gene of interest. In certain embodiments, the method of non-continuous evolution is PANCE. In other embodiments, the method of non-continuous evolution is an antibiotic or plate-based selection method. PANCE uses the same genetic circuit as PACE to activate phage propagation, but instead of continuously diluting a vessel, phage are manually passaged by infecting fresh host-cell culture with an aliquot from the proceeding passage. PANCE is less stringent than PACE because there is little risk of losing a weakly active phage variant during selection, and because the effective rate of phage dilution is much lower.

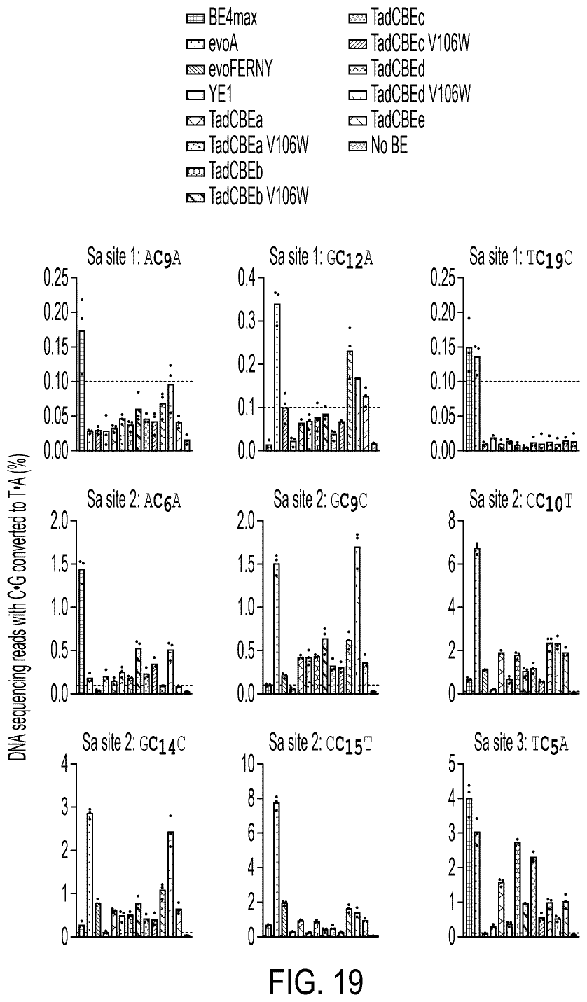

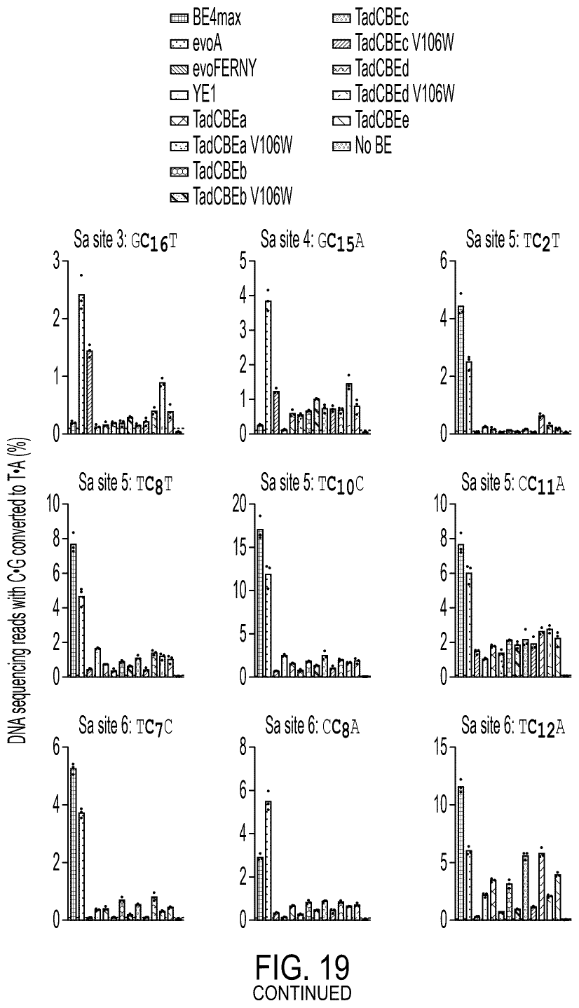

An exemplary PANCE methodology comprises first growing the host strain containing a mutagenesis plasmid of E. coli on 2×YT agar containing 0.5% glucose (w/v) along with appropriate concentrations of antibiotics until optical density reaches A600=0.5-0.6 in a large volume. The cells are re-transformed with the mutagenesis plasmid regularly to ensure the plasmid has not been inactivated. An aliquot of a desired concentration, often 2 mL, is then transferred to a smaller flask, supplemented with 40 mM inducing agent arabinose (Ara) for the mutagenesis plasmid, and infected with the selection phage (SP). To increase the titer level, a drift plasmid may also be provided that enables phage to propagate without passing the selection. Expression is under the control of an inducible promoter and can be turned on with 0-40 ng/mL of anhydrotetracycline. Treated cultures may be split into the desired number of either 2 mL cultures in single culture tubes or 500 μL cultures in a 96-well plate and infected with selection phage (see FIG. 19). These cultures may be incubated at 37° C. for 8-12 h to facilitate phage growth, which is confirmed by determination of the phage titer, and then harvested. Following phage growth, an aliquot of infected cells is used to transfect a subsequent flask containing host E. coli. Supernatant containing evolved phage may isolated and stored at 4° C. This process may be continued until the desired phenotype is evolved for as many transfers as required, while increasing the stringency in stepwise fashion by decreasing the incubation time or titer of phage with which the bacteria is infected. In an exemplary PANCE protocol as provided herein, the process is iterated in 25 culture passages. Reference is made to Suzuki T. et al., Crystal structures reveal an elusive functional domain of pyrrolysyl-tRNA synthetase, Nat Chem Biol. 13(12): 1261-1266 (2017), incorporated herein in its entirety.

In some embodiments, negative selection is applied during a non-continuous evolution method as described herein, by penalizing undesired activities. In some embodiments, this is achieved by causing the undesired activity to interfere with pIII production. For example, expression of an antisense RNA complementary to the gIII RBS and/or start codon is one way of applying negative selection, while expressing a protease (e.g., TEV) and engineering the protease recognition sites into pIII is another.

Other non-continuous selection schemes for gene products having a desired activity are well known to those of skill in the art or will be apparent from the present disclosure. In certain embodiments, following the successful directed evolution of one or more components of the adenine base editor (e.g., a Cas9 domain or a adenosine deaminase domain), methods of making the base editors comprise recombinant protein expression methodologies known to one of ordinary skill in the art.

In some embodiments, the PACE/PANCE methodology comprises (a) a selection phage encoding a mutated TadA8e protein fused to an NpunN intein, (b) a first plasmid encoding an NpuC intein fused to dCas9-UGI, (c) a second plasmid encoding a gIII driven by a T7 or proT7 promoter and encoding an sgRNA, and (d) a third plasmid encoding a T7 RNA polymerase-degron fusion. In certain cases, the T7 RNA polymerase-degron fusion contains a target sequence at the interface between the T7 RNA polymerase and the degron domains. The target sequence may comprise one or more cytosine nucleotides that when edited to uracil insert a STOP codon between the T7 polymerase and degron domains of the T7 RNA polymerase-degron fusion.

In some embodiments, promoters described herein may be a strong promoter making the evolution circuit less stringent. Alternatively, or additionally, in some embodiments, the promoters described herein may be weak promoters, thus making the evolution circuits more stringent.

### Development of Selection Circuit

Various embodiments of the disclosure relate to providing directed evolution methods for modulating selection stringency. The disclosure provides selection circuits for the disclosed directed evolution methods to engineer any of the disclosed base editors or base editor domains (e.g., the cytidine deaminase domains of any of the disclosed base editors). The selection circuits described herein allow for modulating the tolerance of residual adenosine deamination activity.

Selection circuits are conducted using PACE/PANCE methodologies described elsewhere herein. The evolving protein of interest (e.g., TadA-8e) is encoded on a selection phage (SP), which infects E. coli host cells⁴⁶. The E. coli harbor a mutagenesis plasmid (MP) that constantly mutagenizes the phage genome, as well as accessory plasmid(s) (AP), that regulates the expression of gene III, which encodes pIII, a critical protein for phage replication. Since gIII has been removed from the SP genome, only phage that encode evolving variants with the desired activity trigger the production of pIII in E. coli and replicate, resulting in the propagation of active gene variants (e.g., mutant TadA-8e with cytidine deaminase activity). Under constant mutagenesis and dilution (e.g., using PACE), phage lacking the desired activity are rapidly diluted from the selection vessel (“lagoon”), while phage that evolve beneficial mutations persist.

In some embodiments, the TadA-8e deaminase is encoded within the SP and the host E. coli cells contain (i) the MP, (ii) an accessory plasmid that encodes SpCas9, (iii) a self-inactivating T7 RNA polymerase (T7 RNAP) fused to a C-terminal (3′ end) degron tag, and (iv) gene III under T7 RNAP transcriptional control. Upon phage infection, the SP-encoded deaminase is joined to Cas9 by trans-intein splicing to reconstitute the base editor. In order to activate the selection circuit, the base editor must perform C·G-to-T·A to create a stop codon between T7 RNAP and the degron, yielding active T7 RNAP. Degron-free T7 RNAP then transcribes gIII, leading to phage propagation.

In some embodiments, the selection circuit allows for lower selectivity for cytidine over adenosine deamination that is likely to occur during the early stages of evolution. To accomplish this, the non-template (non-coding) strand is edited using the protospacer sequence C₆A₇A₈, which is edited to T₆A₇A₈ to introduce a stop codon upon cytidine deamination (e.g., base editing introduces a TAA stop codon and expression of Degron-free T7 RNAP). Off-target adenosine deamination at protospacer position 7 or position 8 does not prevent codon installation (e.g., results in TGA or TAG stop codons) unless both A₇ and A₈ are converted to Gs (resulting in codon TGG which codes for Trp), making this circuit tolerant to modest levels of adenosine deamination and thus more suitable for early-stage TadA8e evolution (Circuit 1) (see FIG. 1D). In some embodiments, the non-coding strand comprises the protospacer sequence T₄C₆A₇A₈G₉.

Accordingly, in some aspects, provided herein are vector systems comprising: a selection plasmid comprising an isolated nucleic acid encoding an adenosine deaminase comprising, in the following order: an adenosine deaminase protein and a sequence encoding a N-terminal portion of a split intein; a first accessory plasmid comprising, in the following order: a sequence encoding a guide RNA operably controlled by a Lac promoter and a sequence encoding a M13 phage gene III (gIII) peptide operably controlled by a T7 RNA promoter; a second accessory plasmid comprising, in the following order: a sequence encoding a C-terminal portion of a split intein and a sequence encoding a dCas9-UGI fusion; and a third accessory plasmid comprising a non-coding strand and a coding strand, wherein the coding strand comprises an expression construct comprising, in the following order: a promoter, a ribosome binding site, and a sequence encoding a T7 RNA polymerase and a degron tag, wherein the non-coding strand opposite the 3′ end of the sequence encoding a T7 RNA polymerase comprises a CAA sequence. In particular embodiments, the CAA sequence is a protospacer sequence C₆A₇A₈, such as the protospacer sequence T₄C₆A₇A₈G₉. In some embodiments, the adenosine deaminase is TadA-8e. In some embodiments, the split intein is an Npu (Nostoc punctiforme) intein.

In some embodiments, the selection circuit allows for higher selectivity for cytidine over adenosine deamination. To accomplish this, according to some embodiments, the template (coding) strand comprises the protospacer sequence A₆C₅C₄, which upon cytidine deamination at C₅ and/or C₄ yields A₆T₅T_{4,} A₆T₅C_{4,} or A₆C₅T_{4,} and introduces a stop codon (TAA, TAG, TGA), and thus the expression of the Degron-free T7RNAP. However, this selection circuit is intolerant of even a single adenosine deamination as this would results protospacer sequences of G₆T₅T₄, G₆T₅C₄, or G₆C₅T₄, corresponding to non-stop codons CAA, CAG, and CGA. Thus, this circuit is more stringent for selecting against adenosine deamination (Circuit 2) (see FIG. 1D). Accordingly, in some embodiments, a third accessory plasmid is provided comprising a non-coding strand and a coding strand, wherein the coding strand comprises an expression construct comprising, in the following order: a promoter, a ribosome binding site, and a sequence encoding a T7 RNA polymerase and a degron tag, wherein the coding strand, at the 3′ end of the sequence encoding a T7 RNA polymerase, comprises an ACC sequence (e.g., the protospacer sequence A₆C₅C₄).

In some embodiments, the protein to be evolved in Circuit 2 is the product of Circuit 1. In other words, the Circuit 1 may be used to obtain a pool of evolved TadA-8e deaminases within specificity and activity for both adenosine and cytidine bases. These evolved deaminases may be further evolved using Circuit 2 to screen out variants with residual adenosine specificity and activity to yield cytidine deaminases with high specificity toward cytosine bases.

In some embodiments, the selection circuit comprises a selection phage encoding the mutated TadA8e protein fused to an NpuN intein. In certain cases, a first plasmid encodes an NpuC intein fused to dCas9-UGI and a second plasmid encodes a gIII driven by a T7 or proT7 promoter and encodes an sgRNA. In certain embodiments a third plasmid encodes a T7 RNA polymerase-degron fusion. In some embodiments, the T7 RNA polymerase-degron fusion contains a target sequence at the interface between the T7 RNA polymerase and degron domains. The target sequence, in some cases, may contain one or more cytosine nucleotides that when edited to thymine insert a STOP codon between the T7 RNA polymerase and degron domains of the T7 RNA polymerase-degron fusion.

### Vectors

Several aspects of the making and using the base editors of the disclosure relate to vector systems comprising one or more vectors encoding the cytosine base editors (e.g., vectors comprising the polynucleotide encoding the cytosine base editor). Vectors may be designed to clone and/or express the cytosine base editors of the disclosure. Vectors may also be designed to transfect the evolved adenine base editors of the disclosure into one or more cells, e.g., a target diseased eukaryotic cell for treatment with the base editor systems and methods disclosed herein. In some cases, vectors may comprise a polynucleotide encoding an RNA (e.g., a guide RNA).

Vectors may be designed for expression of base editor transcripts (e.g. nucleic acid transcripts, proteins, or enzymes) in prokaryotic or eukaryotic cells. For example, base editor transcripts may be expressed in bacterial cells such as Escherichia coli, insect cells (using baculovirus expression vectors), yeast cells, plant cells, or mammalian cells. Suitable host cells are discussed further in Goeddel, Gene Expression Technology: Methods In Enzymology 185, Academic Press. San Diego, Calif. (1990). Alternatively, expression vectors encoding one or more evolved adenine base editors described herein may be transcribed and translated in vitro, for example using T7 promoter regulatory sequences and T7 polymerase. In some embodiments, the vector comprises a heterologous promoter that drives expression of a polynucleotide encoding the base editor. Vectors encoding the cytosine base editors provided herein may comprise any of the DNA vectors identified below as TadCBEa-eNme2-C-BE4max, TadCBEa-enCjCas9-BE4max, TadCBEa-SpCas9-BE4max, TadCBEa-SaCas9-BE4max, TadCBEa-SpCas9-NG-BE4max. These vectors are provided below.

Exemplary vectors of the disclosure comprise any of the base editor-encoding vectors set forth as SEQ ID NOs: 100-104. In some embodiments, the disclosed vectors comprise a nucleic acid sequence having at least 80%, at least 85%, at least 90%, at least 95%, at least 98%, at least 99%, or at least 99.5% sequence identity to any of SEQ ID NOs: 100-104. In some embodiments, any of the vectors described herein may comprise a nucleic acid sequence having 1-5, 5-10, 10-15, 15-20, 20-25, 25-30, 30-35, 35-40, 40-45, 45-50, or more than 50 nucleotides that differ relative to the sequence of any of SEQ ID NOs: 100-104. These differences may comprise nucleotides that have been inserted, deleted, or substituted relative to any of SEQ ID NOs: 100-104. In some embodiments, the disclosed vectors contain stretches of about 50, about 75, about 100, about 125, about 150, about 175, about 200, about 300, about 400, about 500, or more than 500 consecutive nucleotides in common with any of SEQ ID NOs: 100-104.

TadCBEa-eNme2-C-BE4max vector
(SEQ ID NO: 100)
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggcggtaggcgtgtacggt
gggaggtctatataagcagagctggtttagtgaaccgtcagatccgctagagatccgcggccgctaatacgactcactatagggagag
ccgccaccatgaaacggacagccgacggaagcgagttcgagtcaccaaagaagaagcggaaagtcagttctgaggtggagttttcc
cacgagtactggatgagacatgccctgaccctggccaagagggcacgggatgaggggggggtcctgtgggagccgtgctggtgc
tgaacaatagagtgatcggcgagggctggaacagagccatcggcctgcacgacccaacagcccatgccgaaattatggccctgaga
cagggcggcctggtcatgcagaactacagactgtttgacgccaccctgtacgtgacattcgagccttgcgtgatgtgcgccggcgcca
tgatcaactctaggatcggccgcgtggtgtttggcgtgaggaactcaaaaagaggcgccgcaggctccctgatgaacgtgctgaact
accccggcatgaatcaccgcgtcgaaattaccgagggaatcctggcagatgaatgtgccgccctgctgtgcgatttctatcggatgcct
agacaggtgttcaattctcagaagaaggcccagagctccatcaactctggcggatctagcggaggatcctctggcagcgagacacca
ggaacaagcgagtcagcaacaccagagagcagtggcggcagcagcggcggcagcgcagcattcaagcctaacccaatcaattac
atcctgggactggcaatcggaatcgcatccgtgggatgggctatggtggagatcgacgaggaggggaatcctatccggctgatcgat
ctgggcgtgagagtgtttgagagggccgaggtgccaaagaccggcgattctctggctatggcccggagactggcacggagcgtga
ggcgcctgacacggagaagggcacacaggctgctgagggcacgccggctgctgaagagagagggcgtgctgcaggcagcaga
cttcgatgagaatggcctgatcacgagcttgccaaacaccccctggcagctgagagcagccgccctggacaggaagctgacaccac
tggagtggtctgccgtgctgctgcacctgatcaagcaccgcggctacctgagccagcggaagaacgagggagagacagcagccaa
ggagctgggcgccctgctgaagggagtggccaacaatgcccacgccctgcagaccggcgatttcaggacacctgccgagctggcc
ctgaataagtttgagaaggagtccggccacatcagaaaccagaggggcgactatagccacaccttctcccgcaaggatctgcaggcc
gagctgatcctgctgttcgagaagcagaaggagtttggcaatccacacgtgagcggaggcctgaaggagggaatcgagaccctgct
gatgacacagaggcctgccctgtccggcgacgcagtgcagaagatgctggggcactgcaccctcgagcctacagagccaaaggcc
gccaagaacacctacacagccgagcggtttatctggctgacaaagctgaacaatctgagaatcctggagcagggatccgagaggcc
actgaccgacacagagaggtccaccctgatggatgagccttaccggaagtctaaactgacatatgcccaggccagaaagctgctgg
gcctggaggacaccgccttctttaagggcctgagatacggcaaggataatgccgaggcctccacactgatggagatgaaggcctatc
acgccatctctcgcgccctggagaaggagggcctgaaggacaagaagtcccccctgaacctgagctccgagctgcaggatgagat
cggcaccgccttctctctgtttaagaccgacgaggatatcacaggccgcctgaaggacagggtgcagcctgagatcctggaggccct
gctgaagcacatctctttcgataagtttgtgcagatcagcctgaaggccctgagaaggatcgtgccactgatggagcagggcaagcg
gtacgacgaggcctgcgccgagatctacggcgttcactatggcaagaagaacacagaggagaagatctatctgccccctatccctgc
cgacgagatcagaaatcctgtggtgctgagggccctgtcccaggcaagaaaagtgatcaacggagtggtgcgccggtacggatctc
cagcccggatccacatcgagaccgccagagaagtgggcaagagcttcaaggaccggaaggagatcgcgaagagacaggaggag
aatcgcaaggatcgggagaaggccgccgccaagtttagggagtacttccctaactttgtgggcgagccaaagtctaaggacatcctg
aagctgcgcctgtacgagcagcagcacggcaagtgtctgtatagcggcaaagagatcaatctggtgcggctgaacgagaagggcta
tgtggagatcgatcacgccctgcctttctccagaacctgggacgattcttttaacaataaggtgctggtgctgggcagcgagaaccaga
ataagggcaatcagacaccatacgagtatttcaatggcaaggacaactccagggagtggcaggagttcaaggcccgcgtggagacc
tctagatttcccagtagcaagaagcagcggatcctgctgcagaagttcgacgaggatggctttaaggagtgcaacctgaatgacacca
gatacgtgaaccggttcctgtgccagtttgtggccgatcacatcctgctgaccggcaagggcaagagaagggtggtcgcctctaatgg
ccagatcacaaacctgctgagggggttttggagactgaggaaggtgcgggcagagaatgacagacaccacgcactggatgcagtg
gtggtggcatgcagcaccgtggcaatgcagcagaagatcacaagattcgtgaggtataaggagatgaacgcctttgacggcaagac
cgtcgataaggagacaggcaaggtgctgtaccagaagacccacttcccccagccttgggagttctttgcccaggaagttatgatccgg
gtgttcggcaagccagacggcaagcctgagtttgaggaggccgataccccagagaagctgaggacactgctggcagagaagctgt
ctagcaggccagaggcagtgcacgagtacgtgaccccgctgttcgtgtccagggcacccaatcggaagatgtctggcgcccacaag
gacacactgagaagcgccaagaggtttgtgaagcacaacgagaagatctccgtgaagagagtgtggctgaccgagatcaagctggc
cgatctggagaacatggtgaattacaagaacggcagggagatcgagctgtatgaggccctgaaggcaaggctggaggcctacgga
ggaaatgccaagcaggccttcgacccaaaggataaccccttttataagaagggaggacagctggtgaaggccgtgcgggtggaga
agacccagaaaagcggcgtgctgctgaataagaagaacgcctacacaatcgccgacaatggtgatatggtgagagtggacgtgttct
gtaaggtggataagaagggcaagaatcagtactttatcgtgcctatctatgcctggcaggtggccgagaacatcctgccagacatcgat
tgcaagggctacagaatcgacgatagctatacattctgtttttccctgcacaagtatgacctgatcgccttccagaaggatgagaagtcc
aaggtggagtttgcctactatatcaattgcgactcctctagcggcgggttctacctggcctggcacgataagggcagcagggagcagc
ggtttcgcatctccacccagaatctggcgctgatccagaagtatcaggtgaacgagctgggcaaggagatcaggccatgtcggctga
agaagcgcccacccgtgagcggcgggagcggcgggagcggggggagcactaatctgagcgacatcattgagaaggagactggg
aaacagctggtcattcaggagtccatcctgatgctgcctgaggaggtggaggaagtgatcggcaacaagccagagtctgacatcctg
gtgcacaccgcctacgacgagtccacagatgagaatgtgatgctgctgacctctgacgcccccgagtataagccttgggccctggtca
tccaggattctaacggcgagaataagatcaagatgctgagcggaggatccggaggatctggaggcagcaccaacctgtctgacatca
tcgagaaggagacaggcaagcagctggtcatccaggagagcatcctgatgctgcccgaagaagtcgaagaagtgatcggaaacaa
gcctgagagcgatatcctggtccataccgcctacgacgagagtaccgacgaaaatgtgatgctgctgacatccgacgccccagagta
taagccctgggctctggtcatccaggattccaacggagagaacaaaatcaaaatgctgtctggcggctcaaaaagaaccgccgacgg
cagcgaattcgagcccaagaagaagaggaaagtctaaccggtcatcatcaccatcaccattgagtttaaacccgctgatcagcctcga
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggggggcaggacagcaagggggaggattg
ggaagacaatagcaggcatgctggggatgcggtgggctctatggcttctgaggcggaaagaaccagctggggctcgataccgtcga
cctctagctagagcttggcgtaatcatggtcatagctgtttcctgtgtgaaattgttatccgctcacaattccacacaacatacgagccgga
agcataaagtgtaaagcctagggtgcctaatgagtgagctaactcacattaattgcgttgcgctcactgcccgctttccagtcgggaaac
ctgtcgtgccagctgcattaatgaatcggccaacgcgcggggagaggcggtttgcgtattgggcgcacttccgcttcctcgctcactga
ctcgctgcgctcggtcgttcggctgcggcgagcggtatcagctcactcaaaggcggtaatacggttatccacagaatcaggggataac
gcaggaaagaacatgtgagcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaagataccaggcgtttcccc
ctggaagctccctcgtgcgctctcctgttccgaccctgccgcttaccggatacctgtccgcctttctcccttcgggaagcgtggcgctttc
tcatagctcacgctgtaggtatctcagttcggtgtaggtcgttcgctccaagctgggctgtgtgcacgaaccccccgttcagcccgacc
gctgcgccttatccggtaactatcgtcttgagtccaacccggtaagacacgacttatcgccactggcagcagccactggtaacaggatt
agcagagcgaggtatgtaggcggtgctacagagttcttgaagtggtggcctaactacggctacactagaagaacagtatttggtatctg
cgctctgctgaagccagttaccttcggaaaaagagttggtagctcttgatccggcaaacaaaccaccgctggtagcggtggtttttttgtt
tgcaagcagcagattacgcgcagaaaaaaaggatctcaagaagatcctttgatcttttctacggggtctgacactcagtggaacgaaaa
ctcacgttaagggattttggtcatgagattatcaaaaaggatcttcacctagatccttttaaattaaaaatgaagttttaaatcaatctaaagt
atatatgagtaaacttggtctgacagttaccaatgcttaatcagtgaggcacctatctcagcgatctgtctatttcgttcatccatagttgcct
gactccccgtcgtgtagataactacgatacgggagggcttaccatctggccccagtgctgcaatgataccggggacccacgctcac
cggctccagatttatcagcaataaaccagccagccggaagggccgagcgcagaagtggtcctgcaactttatccgcctccatccagtc
tattaattgttgccgggaagctagagtaagtagttcgccagttaatagtttgcgcaacgttgttgccattgctacaggcatcgtggtgtcac
gctcgtcgtttggtatggcttcattcagctccggttcccaacgatcaaggcgagttacatgatcccccatgttgtgcaaaaaagcggttag
ctccttcggtcctccgatcgttgtcagaagtaagttggccgcagtgttatcactcatggttatggcagcactgcataattctcttactgtcat
gccatccgtaagatgcttttctgtgactggtgagtactcaaccaagtcattctgagaatagtgtatgcggcgaccgagttgctcttgcccg
gcgtcaatacgggataataccgcgccacatagcagaactttaaaagtgctcatcattggaaaacgttcttcggggcgaaaactctcaag
gatcttaccgctgttgagatccagttcgatgtaacccactcgtgcacccaactgatcttcagcatcttttactttcaccagcgtttctgggtg
agcaaaaacaggaaggcaaaatgccgcaaaaaagggaataagggcgacacggaaatgttgaatactcatactcttcctttttcaatatt
attgaagcatttatcagggttattgtctcatgagcggatacatatttgaatgtatttagaaaaataaacaaataggggttccgcgcacatttc
cccgaaaagtgccacctgacgtcgacggatcgggagatcgatctcccgatcccctagggtcgactctcagtacaatctgctctgatgc
cgcatagttaagccagtatctgctccctgcttgtgtgttggaggtcgctgagtagtgcgcgagcaaaatttaagctacaacaaggcaag
gcttgaccgacaattgcatgaagaatctgcttagggttaggcgttttgcgctgcttcgcgatgtacgggccagatatacgcgttgacattg
attattgac
TadCBEa-enCjCas9-BE4max vector
(SEQ ID NO: 101)
attgatgcagtaatcatagcctacgctaacaacagcatcgtgaaagccttctccgatttcaagaaagaacaggagtctaatagcgccga
gttgtacgccaagaaaatttccgaattggactataaaaataagagaaaattcttcgaacccttctccgggtttcgccaaaaggtcttagat
aagatcgacgagattttcgtttccaagcccgaaagaaaaaagccttcaggggcactgcacgaagagacattccgcaaggaagagga
attttaccaatcttacggtggtaaagagggagttctgaaggctctggagcttgggaagatccgcaaggtaaacgggaaaatcgtgaaa
aacggggacatgttcagggtggatatcttcaagcacaaaaagaccaacaagttctacgcagtacccatctacactatggatttcgcttta
aaggttctcccaaataaggcggtggctcgatcgaagaaaggagagatcaaggactggatcttaatggatgaaaattacgagttttgctt
ctcgctctacaaagatagcctgattctgatccagacaaaaaagatgcaggaaccagaatttgtttattataacgccttcacgagcagtac
agtgtccctgattgtgagcaagcatgataacaagttcgagactctgtctaagaatcagaaaatccttttcaagaacgccaacgagaagg
aggtcatcgcaaagtcaattggcatccaaaacctgaaggtgttcgagaaatacatagtgtccgcactcggtgaagtaactaaagccga
atttcgacagcgcgaggattttaagaaaagcggcgggagcggcgggagcggggggagcactaatctgagcgacatcattgagaag
gagactgggaaacagctggtcattcaggagtccatcctgatgctgcctgaggaggtggaggaagtgatcggcaacaagccagagtc
tgacatcctggtgcacaccgcctacgacgagtccacagatgagaatgtgatgctgctgacctctgacgcccccgagtataagccttgg
gccctggtcatccaggattctaacggcgagaataagatcaagatgctgagcggaggatccaaaagaaccgccgacggcagcgaatt
cgagcccaagaagaagaggaaagtctaaccggtcatcatcaccatcaccattgagtttaaacccgctgatcagcctcgactgtgccttc
tagttgccagccatctgttgtttgcccctcccccgtgccttccttgaccctggaaggtgccactcccactgtcctttcctaataaaatgagg
aaattgcatcgcattgtctgagtaggtgtcattctattctggggggtggggggggcaggacagcaagggggaggattgggaagaca
atagcaggcatgctggggatgcggtgggctctatggcttctgaggcggaaagaaccagctggggctcgataccgtcgacctctagct
agagcttggcgtaatcatggtcatagctgtttcctgtgtgaaattgttatccgctcacaattccacacaacatacgagccggaagcataaa
gtgtaaagcctagggtgcctaatgagtgagctaactcacattaattgcgttgcgctcactgcccgctttccagtcgggaaacctgtcgtg
ccagctgcattaatgaatcggccaacgcgcggggagaggcggtttgcgtattgggcgcacttccgcttcctcgctcactgactcgctg
cgctcggtcgttcggctgcggcgagcggtatcagctcactcaaaggcggtaatacggttatccacagaatcaggggataacgcagga
aagaacatgtgagcaaaaggccagcaaaaggccaggaaccgtaaaaaggccgcgttgctggcgtttttccataggctccgcccccct
gacgagcatcacaaaaatcgacgctcaagtcagaggtggcgaaacccgacaggactataaagataccaggcgtttccccctggaag
ctccctcgtgcgctctcctgttccgaccctgccgcttaccggatacctgtccgcctttctcccttcgggaagcgtggcgctttctcatagct
cacgctgtaggtatctcagttcggtgtaggtcgttcgctccaagctgggctgtgtgcacgaaccccccgttcagcccgaccgctgcgc
cttatccggtaactatcgtcttgagtccaacccggtaagacacgacttatcgccactggcagcagccactggtaacaggattagcagag
cgaggtatgtaggcggtgctacagagttcttgaagtggtggcctaactacggctacactagaagaacagtatttggtatctgcgctctgc
tgaagccagttaccttcggaaaaagagttggtagctcttgatccggcaaacaaaccaccgctggtagcggtggtttttttgtttgcaagca
gcagattacgcgcagaaaaaaaggatctcaagaagatcctttgatcttttctacggggtctgacactcagtggaacgaaaactcacgtta
agggattttggtcatgagattatcaaaaaggatcttcacctagatccttttaaattaaaaatgaagttttaaatcaatctaaagtatatatgagt
aaacttggtctgacagttaccaatgcttaatcagtgaggcacctatctcagcgatctgtctatttcgttcatccatagttgcctgactccccg
tcgtgtagataactacgatacgggagggcttaccatctggccccagtgctgcaatgataccgcgggacccacgctcaccggctccag
atttatcagcaataaaccagccagccggaagggccgagcgcagaagtggtcctgcaactttatccgcctccatccagtctattaattgtt
gccgggaagctagagtaagtagttcgccagttaatagtttgcgcaacgttgttgccattgctacaggcatcgtggtgtcacgctcgtcgtt
tggtatggcttcattcagctccggttcccaacgatcaaggcgagttacatgatcccccatgttgtgcaaaaaagcggttagctccttcggt
cctccgatcgttgtcagaagtaagttggccgcagtgttatcactcatggttatggcagcactgcataattctcttactgtcatgccatccgta
agatgcttttctgtgactggtgagtactcaaccaagtcattctgagaatagtgtatgcggcgaccgagttgctcttgcccggcgtcaatac
gggataataccgcgccacatagcagaactttaaaagtgctcatcattggaaaacgttcttcggggcgaaaactctcaaggatcttaccg
ctgttgagatccagttcgatgtaacccactcgtgcacccaactgatcttcagcatcttttactttcaccagcgtttctgggtgagcaaaaac
aggaaggcaaaatgccgcaaaaaagggaataagggcgacacggaaatgttgaatactcatactcttcctttttcaatattattgaagcat
ttatcagggttattgtctcatgagcggatacatatttgaatgtatttagaaaaataaacaaataggggttccgcgcacatttccccgaaaag
tgccacctgacgtcgacggatcgggagatcgatctcccgatcccctagggtcgactctcagtacaatctgctctgatgccgcatagtta
agccagtatctgctccctgcttgtgtgttggaggtcgctgagtagtgcgcgagcaaaatttaagctacaacaaggcaaggcttgaccga
caattgcatgaagaatctgcttagggttaggcgttttgcgctgcttcgcgatgtacgggccagatatacgcgttgacattgattattgacta
gttattaatagtaatcaattacggggtcattagttcatagcccatatatggagttccgcgttacataacttacggtaaatggcccgcctggct
gaccgcccaacgacccccgcccattgacgtcaataatgacgtatgttcccatagtaacgccaatagggactttccattgacgtcaatgg
gtggagtatttacggtaaactgcccacttggcagtacatcaagtgtatcatatgccaagtacgccccctattgacgtcaatgacggtaaat
ggcccgcctggcattatgcccagtacatgaccttatgggactttcctacttggcagtacatctacgtattagtcatcgctattaccatggtg
atgcggttttggcagtacatcaatgggcgtggatagcggtttgactcacggggatttccaagtctccaccccattgacgtcaatgggagt
ttgttttggcaccaaaatcaacgggactttccaaaatgtcgtaacaactccgccccattgacgcaaatgggcggtaggcgtgtacggtg
ggaggtctatataagcagagctggtttagtgaaccgtcagatccgctagagatccgcggccgctaatacgactcactatagggagagc
cgccaccatgaaacggacagccgacggaagcgagttcgagtcaccaaagaagaagcggaaagtcagttctgaggtggagttttccc
acgagtactggatgagacatgccctgaccctggccaagagggcacgggatgaggggggggtcctgtgggagccgtgctggtgct
gaacaatagagtgatcggcgagggctggaacagagccatcggcctgcacgacccaacagcccatgccgaaattatggccctgaga
cagggcggcctggtcatgcagaactacagactgtttgacgccaccctgtacgtgacattcgagccttgcgtgatgtgcgccggcgcca
tgatcaactctaggatcggccgcgtggtgtttggcgtgaggaactcaaaaagaggcgccgcaggctccctgatgaacgtgctgaact
accccggcatgaatcaccgcgtcgaaattaccgagggaatcctggcagatgaatgtgccgccctgctgtgcgatttctatcggatgcct
agacaggtgttcaattctcagaagaaggcccagagctccatcaactctggcggatctagcggaggatcctctggcagcgagacacca
ggaacaagcgagtcagcaacaccagagagcagtggcggcagcagcggcggcagcgcccgcatcctcgctttcgcaatcggaatct
ctagtatcggatgggccttctctgaaaacgacgaactgaaagactgcggcgtgagaatcttcacaaaggttgaaaaccctaaaacagg
cgagtctttagctctgccacgtaggttggcccgctccgcccgaaaaaggtatgctcggcggaaggctcgcctcaaccacttgaagcat
ttgatagctaatgagttcaaactgaactacgaagattaccagtccttcgacgagtcattggcaaaagcctacaaaggcagccttatcagt
ccttatgagttgagatttcgcgcactcaacgaactgctttctaagcaagactttgctagggtcattctgcacatcgcaaaacggcgaggtt
atgacgatatcaagaactccgacgataaagaaaagggagccattctcaaggcgatcaaacagaatgaggaaaaattggcaaactacc
agagtgtgggcgagtatctgtataaagagtatttccagaagtttaaggaaaacagcaaggagtttacaaacgtcagaaataaaaagga
gtcttacgagagatgcatcgcgcagtcattcctcaaagatgagctgaagctgatatttaagaagcaacgcgaatttggtttctcattctcta
agaagttcgaagaggaggttctttccgtggcgttttacaagagggcgctcaaagacttctcccacctggttggtaactgtagtttcttcac
ggatgagaagcgagctcccaaaaattctcccctggctttcatgtttgttgccctgactcggatcattaacctgctgaacaacctgaaaaat
actgaagggatcttgtatacgaaggacgacctaaatgcactcctgaatgaagtgctcaaaaacggaactctaacctataaacagacca
agaaattactggggctctctgacgactacgagttcaagggcgagaagggtacttattttatcgaattcaaaaagtataaggagttcattaa
agcattgggggaacacaacctcagccaggacgatctcaatgaaattgccaaggacatcacgctgattaaagacgagataaaactgaa
aaaggcactggccaagtatgacctcaaccagaaccagatcgactctctgtccaagctggagttcaaagaccacctaaacatatccttca
aagccctgaaactggtcacccctctaatgctcgaaggaaaaaaatacgacgaggcgtgtaatgaactgaatcttaaggtggccatcaa
tgaggataagaaggactttcttccagcctttaacgagacatattacaaagacgaggtcacaaacccggttgtgctgagggccataaaag
agtatcggaaggttctgaatgccctcctgaagaagtacggcaaagtgcacaaaataaatatcgaattggctagggaggtggggaaga
accattctcagcgagcaaagatcgagaaagagcagaatgagaactacaaagccaagaaagacgccgaactggagtgcgaaaagct
ggggcttaaaataaacagtaaaaacatcctgaaattaagattgttcaaagagcaaaaggagttttgcgcctactcaggggaaaaaatca
aaatatcagacctgcaggacgagaaaatgctggagatcgaccatatctatccgtatagcaggtcatttgacgattcctacatgaacaaa
gtgcttgtgtttaccaaacagaaccaagaaaagctgaaccaaaccccctttgaggctttcggaaacgactcagccaagtggcagaaaa
tcgaagtcctagccaagaatctgcctacaaaaaaacaaaagaggattcttgataagaactataaggacaaggaacagaaaaactttaa
agacaggaacctgaatgacacgaggtacattgcgcgactggttctaaactataccaaagactacctggatttcctccctctgagcgacg
acgagaatactaaactgaatgatacccagaaaggctcaaaggtccacgttgaggctaagtccgggatgctgactagcgccctccgcc
acacgtggggcttcagcgccaaagatcggaataatcatcttcatcacgct
TadCBEa-SpCas9-BE4max vector
(SEQ ID NO: 102)
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggcggtaggcgtgtacggt
gggaggtctatataagcagagctggtttagtgaaccgtcagatccgctagagatccgcggccgctaatacgactcactatagggagag
ccgccaccatgaaacggacagccgacggaagcgagttcgagtcaccaaagaagaagcggaaagtcagttctgaggtggagttttcc
cacgagtactggatgagacatgccctgaccctggccaagagggcacgggatgaggggggggtcctgtgggagccgtgctggtgc
tgaacaatagagtgatcggcgagggctggaacagagccatcggcctgcacgacccaacagcccatgccgaaattatggccctgaga
cagggcggcctggtcatgcagaactacagactgtttgacgccaccctgtacgtgacattcgagccttgcgtgatgtgcgccggcgcca
tgatcaactctaggatcggccgcgtggtgtttggcgtgaggaactcaaaaagaggcgccgcaggctccctgatgaacgtgctgaact
accccggcatgaatcaccgcgtcgaaattaccgagggaatcctggcagatgaatgtgccgccctgctgtgcgatttctatcggatgcct
agacaggtgttcaattctcagaagaaggcccagagctccatcaactctggcggatctagcggaggatcctctggcagcgagacacca
ggaacaagcgagtcagcaacaccagagagcagtggcggcagcagcggcggcagcgacaagaagtacagcatcggcctggccat
cggcaccaactctgtgggctgggccgtgatcaccgacgagtacaaggtgcccagcaagaaattcaaggtgctgggcaacaccgac
cggcacagcatcaagaagaacctgatcggagccctgctgttcgacagcggcgaaacagccgaggccacccggctgaagagaacc
gccagaagaagatacaccagacggaagaaccggatctgctatctgcaagagatcttcagcaacgagatggccaaggtggacgaca
gcttcttccacagactggaagagtccttcctggtggaagaggataagaagcacgagcggcaccccatcttcggcaacatcgtggacg
aggtggcctaccacgagaagtaccccaccatctaccacctgagaaagaaactggtggacagcaccgacaaggccgacctgcggct
gatctatctggccctggcccacatgatcaagttccggggccacttcctgatcgagggcgacctgaaccccgacaacagcgacgtgga
caagctgttcatccagctggtgcagacctacaaccagctgttcgaggaaaaccccatcaacgccagcggcgtggacgccaaggcca
tcctgtctgccagactgagcaagagcagacggctggaaaatctgatcgcccagctgcccggcgagaagaagaatggcctgttcgga
aacctgattgccctgagcctgggcctgacccccaacttcaagagcaacttcgacctggccgaggatgccaaactgcagctgagcaag
gacacctacgacgacgacctggacaacctgctggcccagatcggcgaccagtacgccgacctgtttctggccgccaagaacctgtc
cgacgccatcctgctgagcgacatcctgagagtgaacaccgagatcaccaaggcccccctgagcgcctctatgatcaagagatacg
acgagcaccaccaggacctgaccctgctgaaagctctcgtgcggcagcagctgcctgagaagtacaaagagattttcttcgaccaga
gcaagaacggctacgccggctacattgacggcggagccagccaggaagagttctacaagttcatcaagcccatcctggaaaagatg
gacggcaccgaggaactgctcgtgaagctgaacagagaggacctgctgcggaagcagcggaccttcgacaacggcagcatcccc
caccagatccacctgggagagctgcacgccattctgcggcggcaggaagatttttacccattcctgaaggacaaccgggaaaagatc
gagaagatcctgaccttccgcatcccctactacgtgggccctctggccaggggaaacagcagattcgcctggatgaccagaaagag
cgaggaaaccatcaccccctggaacttcgaggaagtggtggacaagggcgcttccgcccagagcttcatcgagcggatgaccaact
tcgataagaacctgcccaacgagaaggtgctgcccaagcacagcctgctgtacgagtacttcaccgtgtataacgagctgaccaaagt
gaaatacgtgaccgagggaatgagaaagcccgccttcctgagcggcgagcagaaaaaggccatcgtggacctgctgttcaagacc
aaccggaaagtgaccgtgaagcagctgaaagaggactacttcaagaaaatcgagtgcttcgactccgtggaaatctccggcgtggaa
gatcggttcaacgcctccctgggcacataccacgatctgctgaaaattatcaaggacaaggacttcctggacaatgaggaaaacgag
gacattctggaagatatcgtgctgaccctgacactgtttgaggacagagagatgatcgaggaacggctgaaaacctatgcccacctgtt
cgacgacaaagtgatgaagcagctgaagcggcggagatacaccggctggggcaggctgagccggaagctgatcaacggcatccg
ggacaagcagtccggcaagacaatcctggatttcctgaagtccgacggcttcgccaacagaaacttcatgcagctgatccacgacga
cagcctgacctttaaagaggacatccagaaagcccaggtgtccggccagggcgatagcctgcacgagcacattgccaatctggccg
gcagccccgccattaagaagggcatcctgcagacagtgaaggtggtggacgagctcgtgaaagtgatgggccggcacaagcccga
gaacatcgtgatcgaaatggccagagagaaccagaccacccagaagggacagaagaacagccgcgagagaatgaagcggatcg
aagagggcatcaaagagctgggcagccagatcctgaaagaacaccccgtggaaaacacccagctgcagaacgagaagctgtacct
gtactacctgcagaatgggcgggatatgtacgtggaccaggaactggacatcaaccggctgtccgactacgatgtggaccatatcgtg
cctcagagctttctgaaggacgactccatcgacaacaaggtgctgaccagaagcgacaagaaccggggcaagagcgacaacgtgc
cctccgaagaggtcgtgaagaagatgaagaactactggcggcagctgctgaacgccaagctgattacccagagaaagttcgacaat
ctgaccaaggccgagagaggcggcctgagcgaactggataaggccggcttcatcaagagacagctggtggaaacccggcagatc
acaaagcacgtggcacagatcctggactcccggatgaacactaagtacgacgagaatgacaagctgatccgggaagtgaaagtgat
caccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaag
gtgtacgacgtgcggaagatgatcgccaagagcgagcaggaaatcggcaaggctaccgccaagtacttcttctacagcaacatcatg
aactttttcaagaccgagattaccctggccaacggcgagatccggaagcggcctctgatcgagacaaacggcgaaaccggggagat
cgtgtgggataagggccgggattttgccaccgtgcggaaagtgctgagcatgccccaagtgaatatcgtgaaaaagaccgaggtgc
agacaggcggcttcagcaaagagtctatcctgcccaagaggaacagcgataagctgatcgccagaaagaaggactgggaccctaa
gaagtacggcggcttcgacagccccaccgtggcctattctgtgctggtggtggccaaagtggaaaagggcaagtccaagaaactga
agagtgtgaaagagctgctggggatcaccatcatggaaagaagcagcttcgagaagaatcccatcgactttctggaagccaagggct
acaaagaagtgaaaaaggacctgatcatcaagctgcctaagtactccctgttcgagctggaaaacggccggaagagaatgctggcct
ctgccggcgaactgcagaagggaaacgaactggccctgccctccaaatatgtgaacttcctgtacctggccagccactatgagaagc
tgaagggctcccccgaggataatgagcagaaacagctgtttgtggaacagcacaagcactacctggacgagatcatcgagcagatc
agcgagttctccaagagagtgatcctggccgacgctaatctggacaaagtgctgtccgcctacaacaagcaccgggataagcccatc
agagagcaggccgagaatatcatccacctgtttaccctgaccaatctgggagcccctgccgccttcaagtactttgacaccaccatcga
ccggaagaggtacaccagcaccaaagaggtgctggacgccaccctgatccaccagagcatcaccggcctgtacgagacacggatc
gacctgtctcagctgggaggtgacagcggcgggagcggcgggagcggggggagcactaatctgagcgacatcattgagaaggag
actgggaaacagctggtcattcaggagtccatcctgatgctgcctgaggaggtggaggaagtgatcggcaacaagccagagtctgac
atcctggtgcacaccgcctacgacgagtccacagatgagaatgtgatgctgctgacctctgacgcccccgagtataagccttgggccc
tggtcatccaggattctaacggcgagaataagatcaagatgctgagcggaggatccggaggatctggaggcagcaccaacctgtctg
acatcatcgagaaggagacaggcaagcagctggtcatccaggagagcatcctgatgctgcccgaagaagtcgaagaagtgatcgg
aaacaagcctgagagcgatatcctggtccataccgcctacgacgagagtaccgacgaaaatgtgatgctgctgacatccgacgcccc
agagtataagccctgggctctggtcatccaggattccaacggagagaacaaaatcaaaatgctgtctggcggctcaaaaagaaccgc
cgacggcagcgaattcgagcccaagaagaagaggaaagtctaaccggtcatcatcaccatcaccattgagtttaaacccgctgatca
gcctcgactgtgccttctagttgccagccatctgttgtttgcccctcccccgtgccttccttgaccctggaaggtgccactcccactgtcct
ttcctaataaaatgaggaaattgcatcgcattgtctgagtaggtgtcattctattctggggggtggggggggcaggacagcaaggggg
aggattgggaagacaatagcaggcatgctggggatgcggtgggctctatggcttctgaggcggaaagaaccagctggggctcgata
ccgtcgacctctagctagagcttggcgtaatcatggtcatagctgtttcctgtgtgaaattgttatccgctcacaattccacacaacatacg
agccggaagcataaagtgtaaagcctagggtgcctaatgagtgagctaactcacattaattgcgttgcgctcactgcccgctttccagtc
gggaaacctgtcgtgccagctgcattaatgaatcggccaacgcgcgggaagaggcggtttgcgtattgggcgcacttccgcttcctcg
ctcactgactcgctgcgctcggtcgttcggctgcggcgagcggtatcagctcactcaaaggcggtaatacggttatccacagaatcag
gggataacgcaggaaagaacatgtgagcaaaaggccagcaaaaggccaggaaccgtaaaaaggccgcgttgctggcgtttttccat
aggctccgcccccctgacgagcatcacaaaaatcgacgctcaagtcagaggtggcgaaacccgacaggactataaagataccagg
cgtttccccctggaagctccctcgtgcgctctcctgttccgaccctgccgcttaccggatacctgtccgcctttctcccttcgggaagcgt
ggcgctttctcatagctcacgctgtaggtatctcagttcggtgtaggtcgttcgctccaagctgggctgtgtgcacgaaccccccgttca
gcccgaccgctgcgccttatccggtaactatcgtcttgagtccaacccggtaagacacgacttatcgccactggcagcagccactggt
aacaggattagcagagcgaggtatgtaggcggtgctacagagttcttgaagtggtggcctaactacggctacactagaagaacagtat
ttggtatctgcgctctgctgaagccagttaccttcggaaaaagagttggtagctcttgatccggcaaacaaaccaccgctggtagcggt
ggtttttttgtttgcaagcagcagattacgcgcagaaaaaaaggatctcaagaaaatcctttgatcttttctacggggtctgacactcagtg
gaacgaaaactcacgttaagggattttggtcatgagattatcaaaaaggatcttcacctagatccttttaaattaaaaatgaagttttaaatc
aatctaaagtatatatgagtaaacttggtctgacagttaccaatgcttaatcagtgaggcacctatctcagcgatctgtctatttcgttcatcc
atagttgcctgactccccgtcgtgtagataactacgatacgggagggcttaccatctggccccagtgctgcaatgataccgcgggacc
cacgctcaccggctccagatttatcagcaataaaccagccagccggaagggccgagcgcagaagtggtcctgcaactttatccgcct
ccatccagtctattaattgttgccgggaagctagagtaagtagttcgccagttaatagtttgcgcaacgttgttgccattgctacaggcatc
gtggtgtcacgctcgtcgtttggtatggcttcattcagctccggttcccaacgatcaaggcgagttacatgatcccccatgttgtgcaaaa
aagcggttagctccttcggtcctccgatcgttgtcagaagtaagttggccgcagtgttatcactcatggttatggcagcactgcataattct
cttactgtcatgccatccgtaagatgcttttctgtgactggtgagtactcaaccaagtcattctgagaatagtgtatgcggcgaccgagttg
ctcttgcccggcgtcaatacgggataataccgcgccacatagcagaactttaaaagtgctcatcattggaaaacgttcttcggggcgaa
aactctcaaggatcttaccgctgttgagatccagttcgatgtaacccactcgtgcacccaactgatcttcagcatcttttactttcaccagc
gtttctgggtgagcaaaaacaggaaggcaaaatgccgcaaaaaagggaataagggcgacacggaaatgttgaatactcatactcttc
ctttttcaatattattgaagcatttatcagggttattgtctcatgagcggatacatatttgaatgtatttagaaaaataaacaaataggggttcc
gcgcacatttccccgaaaagtgccacctgacgtcgacggatcgggagatcgatctcccgatcccctagggtcgactctcagtacaatc
tgctctgatgccgcatagttaagccagtatctgctccctgcttgtgtgttggaggtcgctgagtagtgcgcgagcaaaatttaagctacaa
caaggcaaggcttgaccgacaattgcatgaagaatctgcttagggttaggcgttttgcgctgcttcgcgatgtacgggccagatatacg
cgttgacattgattattgac
TadCBEa-SaCas9-BE4max vector
(SEQ ID NO: 103)
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggcggtaggcgtgtacggt
gggaggtctatataagcagagctggtttagtgaaccgtcagatccgctagagatccgcggccgctaatacgactcactatagggagag
ccgccaccatgaaacggacagccgacggaagcgagttcgagtcaccaaagaagaagcggaaagtcagttctgaggtggagttttcc
cacgagtactggatgagacatgccctgaccctggccaagagggcacgggatgaggggggggtcctgtgggagccgtgctggtgc
tgaacaatagagtgatcggcgagggctggaacagagccatcggcctgcacgacccaacagcccatgccgaaattatggccctgaga
cagggcggcctggtcatgcagaactacagactgtttgacgccaccctgtacgtgacattcgagccttgcgtgatgtgcgccggcgcca
tgatcaactctaggatcggccgcgtggtgtttggcgtgaggaactcaaaaagaggcgccgcaggctccctgatgaacgtgctgaact
accccggcatgaatcaccgcgtcgaaattaccgagggaatcctggcagatgaatgtgccgccctgctgtgcgatttctatcggatgcct
agacaggtgttcaattctcagaagaaggcccagagctccatcaactctggcggatctagcggaggatcctctggcagcgagacacca
ggaacaagcgagtcagcaacaccagagagcagtggcggcagcagcggcggcagcgggaagcgaaattacattctggggctggc
cattggcattacatcagtgggctatggcatcattgactacgagacaagggacgtgatcgacgccggcgtgagactgttcaaggaggc
caacgtggagaacaatgagggccggagatccaagaggggagcaaggcgcctgaagcggagaaggcgccacagaatccagaga
gtgaagaagctgctgttcgattacaacctgctgaccgaccactccgagctgtctggcatcaatccttatgaggccagagtgaagggcct
gtcccagaagctgtctgaggaggagtttagcgccgccctgctgcacctggcaaagaggagaggcgtgcacaacgtgaatgaggtg
gaggaggacaccggcaacgagctgtccacaaaggagcagatcagccgcaattccaaggccctggaggagaagtatgtggccgag
ctgcagctggagcggctgaagaaggatggcgaggtgaggggctccatcaatcgcttcaagacctctgactacgtgaaggaggccaa
gcagctgctgaaggtgcagaaggcctaccaccagctggatcagtcctttatcgatacatatatcgacctgctggagacaaggcgcaca
tactatgagggaccaggagagggctctcccttcggctggaaggacatcaaggagtggtacgagatgctgatgggccactgcacctat
tttccagaggagctgagaagcgtgaagtacgcctataacgccgatctgtacaacgccctgaatgacctgaacaacctggtcatcacca
gggatgagaacgagaagctggagtactatgagaagttccagatcatcgagaacgtgttcaagcagaagaagaagcctacactgaag
cagatcgccaaggagatcctggtgaacgaggaggacatcaagggctaccgcgtgacctccacaggcaagccagagttcaccaatct
gaaggtgtatcacgatatcaaggacatcacagcccggaaggagatcatcgagaacgccgagctgctggatcagatcgccaagatcc
tgaccatctatcagagctccgaggacatccaggaggagctgaccaacctgaatagcgagctgacacaggaggagatcgagcagatc
agcaatctgaagggctacaccggcacacacaacctgagcctgaaggccatcaatctgatcctggatgagctgtggcacacaaacga
caatcagatcgccatctttaaccggctgaagctggtgccaaagaaggtggacctgtcccagcagaaggagatcccaaccacactggt
ggacgatttcatcctgtctcccgtggtgaagcggagcttcatccagagcatcaaagtgatcaacgccatcatcaagaagtacggcctgc
ccaatgatatcatcatcgagctggccagggagaagaactccaaggacgcccagaagatgatcaatgagatgcagaagaggaaccg
ccagaccaatgagcggatcgaggagatcatcagaaccacaggcaaggagaacgccaagtacctgatcgagaagatcaagctgcac
gatatgcaggagggcaagtgtctgtattctctggaggccatccctctggaggacctgctgaacaatccattcaactacgaggtggatca
catcatcccccggagcgtgagcttcgacaattcttttaacaataaggtgctggtgaagcaggaggagaacagcaagaagggcaatag
gacccctttccagtacctgtctagctccgattctaagatcagctacgagacattcaagaagcacatcctgaatctggccaagggcaagg
gccgcatcagcaagaccaagaaggagtacctgctggaggagcgggacatcaacagattctccgtgcagaaggacttcatcaaccgg
aatctggtggacaccagatacgccacacgcggcctgatgaatctgctgcggtcttatttcagagtgaacaatctggatgtgaaggtaaa
gagcatcaacggcggcttcacctcctttctgcggagaaagtggaagtttaagaaggagcgcaacaagggctataagcaccacgccg
aggatgccctgatcatcgccaatgccgacttcatctttaaggagtggaagaagctggacaaggccaagaaagtgatggagaaccaga
tgttcgaggagaagcaggccgagagcatgcccgagatcgagacagagcaggagtacaaggagattttcatcacacctcaccagatc
aagcacatcaaggacttcaaggactacaagtattctcacagggtggataagaagcccaaccgcgagctgatcaatgacaccctgtata
gcacacggaaggacgataagggcaataccctgatcgtgaacaatctgaacggcctgtacgacaaggataatgacaagctgaagaag
ctgatcaacaagtctcccgagaagctgctgatgtaccaccacgatcctcagacatatcagaagctgaagctgatcatggagcagtacg
gcgacgagaagaacccactgtataagtactatgaggagacaggcaactacctgacaaagtatagcaagaaggataatggccccgtg
atcaagaagatcaagtactatggcaacaagctgaatgcccacctggacatcaccgacgattaccctaactctcgcaataaggtggtga
agctgagcctgaagccataccggttcgacgtgtacctggacaacggcgtgtataagtttgtgacagtgaagaatctggatgtgatcaag
aaggagaactactatgaggtgaacagcaagtgctacgaggaggccaagaagctgaagaagatcagcaaccaggccgagttcatcg
cctctttttacaacaatgacctgatcaagatcaatggcgagctgtatagagtgatcggcgtgaacaatgatctgctgaacagaatcgaag
tgaatatgatcgacatcacctacagggagtatctggagaacatgaatgataagaggccccctcgcatcatcaagaccatcgcctctaag
acacagagcatcaagaagtacagcacagacatcctggggaacctgtatgaagtcaagagcaagaaacatcctcagattatcaagaaa
ggcagcggcgggagcggcgggagcggggggagcactaatctgagcgacatcattgagaaggagactgggaaacagctggtcatt
caggagtccatcctgatgctgcctgaggaggtggaggaagtgatcggcaacaagccagagtctgacatcctggtgcacaccgccta
cgacgagtccacagatgagaatgtgatgctgctgacctctgacgcccccgagtataagccttgggccctggtcatccaggattctaac
ggcgagaataagatcaagatgctgagcggaggatccggaggatctggaggcagcaccaacctgtctgacatcatcgagaaggaga
caggcaagcagctggtcatccaggagagcatcctgatgctgcccgaagaagtcgaagaagtgatcggaaacaagcctgagagcga
tatcctggtccataccgcctacgacgagagtaccgacgaaaatgtgatgctgctgacatccgacgccccagagtataagccctgggct
ctggtcatccaggattccaacggagagaacaaaatcaaaatgctgtctggcggctcaaaaagaaccgccgacggcagcgaattcga
gcccaagaagaagaggaaagtctaaccggtcatcatcaccatcaccattgagtttaaacccgctgatcagcctcgactgtgccttctag
ttgccagccatctgttgtttgcccctcccccgtgccttccttgaccctggaaggtgccactcccactgtcctttcctaataaaatgaggaaa
ttgcatcgcattgtctgagtaggtgtcattctattctggggggtggggggggcaggacagcaagggggaggattgggaagacaata
gcaggcatgctggggatgcggtgggctctatggcttctgaggcggaaagaaccagctggggctcgataccgtcgacctctagctaga
gcttggcgtaatcatggtcatagctgtttcctgtgtgaaattgttatccgctcacaattccacacaacatacgagccggaagcataaagtg
taaagcctagggtgcctaatgagtgagctaactcacattaattgcgttgcgctcactgcccgctttccagtcgggaaacctgtcgtgcca
gctgcattaatgaatcggccaacgcgcgggaagaggcggtttgcgtattgggcgcacttccgcttcctcgctcactgactcgctgcgct
cggtcgttcggctgcggcgagcggtatcagctcactcaaaggcggtaatacggttatccacagaatcaggggataacgcaggaaag
aacatgtgagcaaaaggccagcaaaaggccaggaaccgtaaaaaggccgcgttgctggcgtttttccataggctccgcccccctgac
gagcatcacaaaaatcgacgctcaagtcagaggtggcgaaacccgacaggactataaagataccaggcgtttccccctggaagctc
cctcgtgcgctctcctgttccgaccctgccgcttaccggatacctgtccgcctttctcccttcgggaagcgtggcgctttctcatagctca
cgctgtaggtatctcagttcggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggtaacaggattagcagagcg
aggtatgtaggcggtgctacagagttcttgaagtggtggcctaactacggctacactagaagaacagtatttggtatctgcgctctgctg
aagccagttaccttcggaaaaagagttggtagctcttgatccggcaaacaaaccaccgctggtagcggtggtttttttgtttgcaagcag
cagattacgcgcagaaaaaaaggatctcaagaaaatcctttgatcttttctacggggtctgacactcagtggaacgaaaactcacgttaa
gggattttggtcatgagattatcaaaaaggatcttcacctagatccttttaaattaaaaatgaagttttaaatcaatctaaagtatatatgagta
aacttggtctgacagttaccaatgcttaatcagtgaggcacctatctcagcgatctgtctatttcgttcatccatagttgcctgactccccgt
cgtgtagataactacgatacgggagggcttaccatctggccccagtgctgcaatgataccgcgggacccacgctcaccggctccaga
tttatcagcaataaaccagccagccggaagggccgagcgcagaagtggtcctgcaactttatccgcctccatccagtctattaattgttg
ccgggaagctagagtaagtagttcgccagttaatagtttgcgcaacgttgttgccattgctacaggcatcgtggtgtcacgctcgtcgttt
ggtatggcttcattcagctccggttcccaacgatcaaggcgagttacatgatcccccatgttgtgcaaaaaagcggttagctccttcggt
cctccgatcgttgtcagaagtaagttggccgcagtgttatcactcatggttatggcagcactgcataattctcttactgtcatgccatccgta
agatgcttttctgtgactggtgagtactcaaccaagtcattctgagaatagtgtatgcggcgaccgagttgctcttgcccggcgtcaatac
gggataataccgcgccacatagcagaactttaaaagtgctcatcattggaaaacgttcttcggggcgaaaactctcaaggatcttaccg
ctgttgagatccagttcgatgtaacccactcgtgcacccaactgatcttcagcatcttttactttcaccagcgtttctgggtgagcaaaaac
aggaaggcaaaatgccgcaaaaaagggaataagggcgacacggaaatgttgaatactcatactcttcctttttcaatattattgaagcat
ttatcagggttattgtctcatgagcggatacatatttgaatgtatttagaaaaataaacaaataggggttccgcgcacatttccccgaaaag
tgccacctgacgtcgacggatcgggagatcgatctcccgatcccctagggtcgactctcagtacaatctgctctgatgccgcatagtta
agccagtatctgctccctgcttgtgtgttggaggtcgctgagtagtgcgcgagcaaaatttaagctacaacaaggcaaggcttgaccga
caattgcatgaagaatctgcttagggttaggcgttttgcgctgcttcgcgatgtacgggccagatatacgcgttgacattgattattgac
TadCBEa-SpCas9-NG-BE4max vector
(SEQ ID NO: 104)
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggcggtaggcgtgtacggt
gggaggtctatataagcagagctggtttagtgaaccgtcagatccgctagagatccgcggccgctaatacgactcactatagggagag
ccgccaccatgaaacggacagccgacggaagcgagttcgagtcaccaaagaagaagcggaaagtcagttctgaggtggagttttcc
cacgagtactggatgagacatgccctgaccctggccaagagggcacgggatgaggggggggtcctgtgggagccgtgctggtgc
tgaacaatagagtgatcggcgagggctggaacagagccatcggcctgcacgacccaacagcccatgccgaaattatggccctgaga
cagggcggcctggtcatgcagaactacagactgtttgacgccaccctgtacgtgacattcgagccttgcgtgatgtgcgccggcgcca
tgatcaactctaggatcggccgcgtggtgtttggcgtgaggaactcaaaaagaggcgccgcaggctccctgatgaacgtgctgaact
accccggcatgaatcaccgcgtcgaaattaccgagggaatcctggcagatgaatgtgccgccctgctgtgcgatttctatcggatgcct
agacaggtgttcaattctcagaagaaggcccagagctccatcaactctggcggatctagcggaggatcctctggcagcgagacacca
ggaacaagcgagtcagcaacaccagagagcagtggcggcagcagcggcggcagcgacaagaagtacagcatcggcctggccat
cggcaccaactctgtgggctgggccgtgatcaccgacgagtacaaggtgcccagcaagaaattcaaggtgctgggcaacaccgac
cggcacagcatcaagaagaacctgatcggagccctgctgttcgacagcggcgaaacagccgaggccacccggctgaagagaacc
gccagaagaagatacaccagacggaagaaccggatctgctatctgcaagagatcttcagcaacgagatggccaaggtggacgaca
gcttcttccacagactggaagagtccttcctggtggaagaggataagaagcacgagcggcaccccatcttcggcaacatcgtggacg
aggtggcctaccacgagaagtaccccaccatctaccacctgagaaagaaactggtggacagcaccgacaaggccgacctgcggct
gatctatctggccctggcccacatgatcaagttccggggccacttcctgatcgagggcgacctgaaccccgacaacagcgacgtgga
caagctgttcatccagctggtgcagacctacaaccagctgttcgaggaaaaccccatcaacgccagcggcgtggacgccaaggcca
tcctgtctgccagactgagcaagagcagacggctggaaaatctgatcgcccagctgcccggcgagaagaagaatggcctgttcgga
aacctgattgccctgagcctgggcctgacccccaacttcaagagcaacttcgacctggccgaggatgccaaactgcagctgagcaag
gacacctacgacgacgacctggacaacctgctggcccagatcggcgaccagtacgccgacctgtttctggccgccaagaacctgtc
cgacgccatcctgctgagcgacatcctgagagtgaacaccgagatcaccaaggcccccctgagcgcctctatgatcaagagatacg
acgagcaccaccaggacctgaccctgctgaaagctctcgtgcggcagcagctgcctgagaagtacaaagagattttcttcgaccaga
gcaagaacggctacgccggctacattgacggcggagccagccaggaagagttctacaagttcatcaagcccatcctggaaaagatg
gacggcaccgaggaactgctcgtgaagctgaacagagaggacctgctgcggaagcagcggaccttcgacaacggcagcatcccc
caccagatccacctgggagagctgcacgccattctgcggcggcaggaagatttttacccattcctgaaggacaaccgggaaaagatc
gagaagatcctgaccttccgcatcccctactacgtgggccctctggccaggggaaacagcagattcgcctggatgaccagaaagag
cgaggaaaccatcaccccctggaacttcgaggaagtggtggacaagggcgcttccgcccagagcttcatcgagcggatgaccaact
tcgataagaacctgcccaacgagaaggtgctgcccaagcacagcctgctgtacgagtacttcaccgtgtataacgagctgaccaaagt
gaaatacgtgaccgagggaatgagaaagcccgccttcctgagcggcgagcagaaaaaggccatcgtggacctgctgttcaagacc
aaccggaaagtgaccgtgaagcagctgaaagaggactacttcaagaaaatcgagtgcttcgactccgtggaaatctccggcgtggaa
gatcggttcaacgcctccctgggcacataccacgatctgctgaaaattatcaaggacaaggacttcctggacaatgaggaaaacgag
gacattctggaagatatcgtgctgaccctgacactgtttgaggacagagagatgatcgaggaacggctgaaaacctatgcccacctgtt
cgacgacaaagtgatgaagcagctgaagcggcggagatacaccggctggggcaggctgagccggaagctgatcaacggcatccg
ggacaagcagtccggcaagacaatcctggatttcctgaagtccgacggcttcgccaacagaaacttcatgcagctgatccacgacga
cagcctgacctttaaagaggacatccagaaagcccaggtgtccggccagggcgatagcctgcacgagcacattgccaatctggccg
gcagccccgccattaagaagggcatcctgcagacagtgaaggtggtggacgagctcgtgaaagtgatgggccggcacaagcccga
gaacatcgtgatcgaaatggccagagagaaccagaccacccagaagggacagaagaacagccgcgagagaatgaagcggatcg
aagagggcatcaaagagctgggcagccagatcctgaaagaacaccccgtggaaaacacccagctgcagaacgagaagctgtacct
gtactacctgcagaatggggggatatgtacgtggaccaggaactggacatcaaccggctgtccgactacgatgtggaccatatcgtg
cctcagagctttctgaaggacgactccatcgacaacaaggtgctgaccagaagcgacaagaaccggggcaagagcgacaacgtgc
cctccgaagaggtcgtgaagaagatgaagaactactggcggcagctgctgaacgccaagctgattacccagagaaagttcgacaat
ctgaccaaggccgagagaggcggcctgagcgaactggataaggccggcttcatcaagagacagctggtggaaacccggcagatc
acaaagcacgtggcacagatcctggactcccggatgaacactaagtacgacgagaatgacaagctgatccgggaagtgaaagtgat
caccctgaagtccaagctggtgtccgatttccggaaggatttccagttttacaaagtgcgcgagatcaacaactaccaccacgcccacg
acgcctacctgaacgccgtcgtgggaaccgccctgatcaaaaagtaccctaagctggaaagcgagttcgtgtacggcgactacaag
gtgtacgacgtgcggaagatgatcgccaagagcgagcaggaaatcggcaaggctaccgccaagtacttcttctacagcaacatcatg
aactttttcaagaccgagattaccctggccaacggcgagatccggaagcggcctctgatcgagacaaacggcgaaaccggggagat
cgtgtgggataagggccgggattttgccaccgtgcggaaagtgctgagcatgccccaagtgaatatcgtgaaaaagaccgaggtgc
agacaggcggcttcagcaaagagtctatccggcccaagaggaacagcgataagctgatcgccagaaagaaggactgggaccctaa
gaagtacggcggcttcgtgagccccaccgtggcctattctgtgctggtggtggccaaagtggaaaagggcaagtccaagaaactgaa
gagtgtgaaagagctgctggggatcaccatcatggaaagaagcagcttcgagaagaatcccatcgactttctggaagccaagggcta
caaagaagtgaaaaaggacctgatcatcaagctgcctaagtactccctgttcgagctggaaaacggccggaagagaatgctggcctc
tgcccggttcctgcagaagggaaacgaactggccctgccctccaaatatgtgaacttcctgtacctggccagccactatgagaagctg
aagggctcccccgaggataatgagcagaaacagctgtttgtggaacagcacaagcactacctggacgagatcatcgagcagatcag
cgagttctccaagagagtgatcctggccgacgctaatctggacaaagtgctgtccgcctacaacaagcaccgggataagcccatcag
agagcaggccgagaatatcatccacctgtttaccctgaccaatctgggagcccctcgggccttcaagtactttgacaccaccatcgacc
ggaaggtgtaccggagcaccaaagaggtgctggacgccaccctgatccaccagagcatcaccggcctgtacgagacacggatcga
cctgtctcagctgggaggtgacagcggcgggagcggcgggagcggggggagcactaatctgagcgacatcattgagaaggagac
tgggaaacagctggtcattcaggagtccatcctgatgctgcctgaggaggtggaggaagtgatcggcaacaagccagagtctgacat
cctggtgcacaccgcctacgacgagtccacagatgagaatgtgatgctgctgacctctgacgcccccgagtataagccttgggccctg
gtcatccaggattctaacggcgagaataagatcaagatgctgagcggaggatccggaggatctggaggcagcaccaacctgtctga
catcatcgagaaggagacaggcaagcagctggtcatccaggagagcatcctgatgctgcccgaagaagtcgaagaagtgatcgga
aacaagcctgagagcgatatcctggtccataccgcctacgacgagagtaccgacgaaaatgtgatgctgctgacatccgacgcccca
gagtataagccctgggctctggtcatccaggattccaacggagagaacaaaatcaaaatgctgtctggcggctcaaaaagaaccgcc
gacggcagcgaattcgagcccaagaagaagaggaaagtctaaccggtcatcatcaccatcaccattgagtttaaacccgctgatcag
cctcgactgtgccttctagttgccagccatctgttgtttgcccctcccccgtgccttccttgaccctggaaggtgccactcccactgtccttt
cctaataaaatgaggaaattgcatcgcattgtctgagtaggtgtcattctattctggggggtggggggggcaggacagcaaggggga
ggattgggaagacaatagcaggcatgctggggatgcggtgggctctatggcttctgaggcggaaagaaccagctggggctcgatac
cgtcgacctctagctagagcttggcgtaatcatggtcatagctgtttcctgtgtgaaattgttatccgctcacaattccacacaacatacga
gccggaagcataaagtgtaaagcctagggtgcctaatgagtgagctaactcacattaattgcgttgcgctcactgcccgctttccagtcg
ggaaacctgtcgtgccagctgcattaatgaatcggccaacgcgcggggagaggcggtttgcgtattgggcgcacttccgcttcctcgc
tcactgactcgctgcgctcggtcgttcggctgcggcgagcggtatcagctcactcaaaggcggtaatacggttatccacagaatcagg
ggataacgcaggaaagaacatgtgagcaaaaggccagcaaaaggccaggaaccgtaaaaaggccgcgttgctggcgtttttccata
ggctccgcccccctgacgagcatcacaaaaatcgacgctcaagtcagaggtggcgaaacccgacaggactataaagataccaggc
gtttccccctggaagctccctcgtgcgctctcctgttccgaccctgccgcttaccggatacctgtccgcctttctcccttcgggaagcgtg
gcgctttctcatagctcacgctgtaggtatctcagttcggtgtaggtcgttcgctccaagctgggctgtgtgcacgaaccccccgttcag
cccgaccgctgcgccttatccggtaactatcgtcttgagtccaacccggtaagacacgacttatcgccactggcagcagccactggta
acaggattagcagagcgaggtatgtaggcggtgctacagagttcttgaagtggtggcctaactacggctacactagaagaacagtattt
ggtatctgcgctctgctgaagccagttaccttcggaaaaagagttggtagctcttgatccggcaaacaaaccaccgctggtagcggtg
gtttttttgtttgcaagcagcagattacgcgcagaaaaaaaggatctcaagaagatcctttgatcttttctacggggtctgacactcagtgg
aacgaaaactcacgttaagggattttggtcatgagattatcaaaaaggatcttcacctagatccttttaaattaaaaatgaagttttaaatca
atctaaagtatatatgagtaaacttggtctgacagttaccaatgcttaatcagtgaggcacctatctcagcgatctgtctatttcgttcatcca
tagttgcctgactccccgtcgtgtagataactacgatacgggagggcttaccatctggccccagtgctgcaatgataccgcgggaccc
acgctcaccggctccagatttatcagcaataaaccagccagccggaagggccgagcgcagaagtggtcctgcaactttatccgcctc
catccagtctattaattgttgccgggaagctagagtaagtagttcgccagttaatagtttgcgcaacgttgttgccattgctacaggcatcg
tggtgtcacgctcgtcgtttggtatggcttcattcagctccggttcccaacgatcaaggcgagttacatgatcccccatgttgtgcaaaaa
agcggttagctccttcggtcctccgatcgttgtcagaagtaagttggccgcagtgttatcactcatggttatggcagcactgcataattctc
ttactgtcatgccatccgtaagatgcttttctgtgactggtgagtactcaaccaagtcattctgagaatagtgtatgcggcgaccgagttgc
tcttgcccggcgtcaatacgggataataccgcgccacatagcagaactttaaaagtgctcatcattggaaaacgttcttcggggcgaaa
actctcaaggatcttaccgctgttgagatccagttcgatgtaacccactcgtgcacccaactgatcttcagcatcttttactttcaccagcgt
ttctgggtgagcaaaaacaggaaggcaaaatgccgcaaaaaagggaataagggcgacacggaaatgttgaatactcatactcttcctt
tttcaatattattgaagcatttatcagggttattgtctcatgagcggatacatatttgaatgtatttagaaaaataaacaaataggggttccgc
gcacatttccccgaaaagtgccacctgacgtcgacggatcgggagatcgatctcccgatcccctagggtcgactctcagtacaatctg
ctctgatgccgcatagttaagccagtatctgctccctgcttgtgtgttggaggtcgctgagtagtgcgcgagcaaaatttaagctacaaca
aggcaaggcttgaccgacaattgcatgaagaatctgcttagggttaggcgttttgcgctgcttcgcgatgtacgggccagatatacgcg
ttgacattgattattgac

Vectors may be introduced and propagated in a prokaryotic cells. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-base editors.

Fusion expression vectors also may be used to express the TadA-CD base editors of the disclosure. Such vectors generally add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the base editor. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

### Methods of Using TadA-Derived Cytosine Base Editors

Some aspects of the disclosure provide methods of using the TadA-CD base editors described herein, such as, for example, the editing of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an TadA-CD domain) and a guide nucleic acid (e.g., gRNA), wherein the target region comprises a targeted nucleobase pair. As a result of embodiments of these methods, strand separation of said target region is induced, a first nucleobase of said target nucleobase pair in a single strand of the target region is converted to a second nucleobase, and no more than one strand of said target region is cut (or nicked), wherein a third nucleobase complementary to the first nucleobase base is replaced by a fourth nucleobase complementary to the second nucleobase.

In some aspects, the invention relates to a method comprising contacting a nucleic acid with any of the base editors (e.g., TadA-CD) or complexes described herein. The nucleic acid, in some embodiments, comprises a target sequence in the genome of a cell (e.g., DNA). In some cases, the nucleic acid is DNA. The DNA may be single-stranded or double-stranded.

The target sequence may, according to some embodiments, comprise a sequence associated with a disease or disorder. In some embodiments, the disease or disorder is HIV/AIDS. In some embodiments, the disease or disorder is sickle cell, or a related hemoglobinopathy. In some instances, the target sequence may comprise a target gene sequence. For example, in some embodiments, the target sequence comprises a sequence in the BCL11A enhancer or the CCR5 or CXCR4 genes (e.g., a subsequence within the gene). The target sequence, may in some instances, comprises a point mutation associated with the disease or disorder (e.g., mutations in CCR5 decrease HIV infectivity).

In some cases, contacting the nucleic acid comprising the target sequence containing a point mutation to one or more of the base editors described herein results in a correction of the point mutation. In some embodiments, the target sequence comprises a T to C point mutation associated with the disease or disorder may be corrected, for example, by deamination of the mutant C base using the TadCBEs described herein, resulting in a sequence that is not associated with the disease or disorder. In certain embodiments, the target sequence comprises an A to G point mutation associated with a disease or disorder, and deamination of the C base that is complementary to the G base of the A to G point mutation results in a sequence that is not associated with the disease or disorder. In some cases, the target sequence (e.g., encoding a protein) comprises a point mutation in a codon (e.g., a mutant C codon) that changes the amino acid encoded by the mutant codon as compared to a wild-type codon. As such, deamination of the mutant C codon, for example, using any of the disclosed TadCBEs, may be used to change the amino acid encoded by the mutant codon to the wild-type amino acid.

In some embodiments, the target sequence may comprise one or more C:T or A:G point mutations. Deamination of a cytosine base, using the TadCBEs described herein, that is complementary to a guanine base of an A to G point mutation results in a change of the amino acid encoded by the mutant codon. Similarly, use of TadCBEs to deaminate the A base that is complementary to the T base of the C to T point mutation results in the codon encoding a wild-type amino acid. In some embodiments, the target sequence comprises the DNA sequence 5′-NCN-3′ where N is A, T, C, or G. In other embodiments, the target sequence comprises the DNA sequence 5′-NCN-3′ where the cytidine is deaminated. Those of skill in the art will understand, that follow normal innate repair process, the deaminated cytidine (e.g., uracil) in the DNA sequence 5′-NCN-3′ is changed to T (DNA polymerase reads uracil as thymidine).

Described in more detail below, deamination of a target sequence may, in some cases, result in removal and/or insertion of a stop codon. Without wishing to be bound by theory, those of skill in the art, will understand that acceptable stop codons comprise the nucleic acid sequences 5′-TAG-3′, 5′-TAA-3′, or 5′-TGA-3′.

In some embodiments, the target sequence comprises a first nucleobase comprising cytidine. In some embodiments, the sequence comprises a second nucleobase comprising deaminated cytidine. In some embodiments, the sequence comprises a third nucleobase comprising a guanine. In some embodiments, the target sequence comprises a fourth nucleobase comprising a thymine. In some embodiments, the second nucleobase is replaced with a fifth nucleobase that is complementary to the fourth nucleobase, thereby generating an intended edited base pair (e.g., A:T to G:C). In some embodiments, the fifth nucleobase is a adenine. In some embodiments, at least 5% of the intended base pairs are edited.

In some embodiments, the TadCBEs may be used to deaminate a cytidine to a uracil. In some cases, deamination results in the introduction and/or removal of a splice site. In other cases, deamination results in the introduction of a mutation in a gene promoter, for example, to increase and/or decrease the transcription of a gene operably linked to the gene promoter. In certain embodiments, the deamination results in the introduction of a mutation in a gene repressor, for example, to increase and/or decrease the transcription of a gene operably linked to the gene repressor.

In some embodiments, contacting a nucleic acid with any of the base editors (e.g., TadA-CD) or complexes described herein is performed in vivo in a subject (e.g., using a vector such as an AAV). In some cases, the subjects have been diagnosed with a disease or disorder associated with a point mutation. Exemplary diseases or disorders include HIV or sickle cell disease. In other embodiments, the step of contacting the nucleic acid with any of the base editors or complexes described herein is performed in vitro or ex vivo.

In some embodiments, the first nucleobase is a cytosine. In some embodiments, the second nucleobase is a deaminated cytosine, or inosine. In some embodiments, the third nucleobase is a guanine. In some embodiments, the fourth nucleobase is an adenine. In some embodiments, the method further comprises replacing the second nucleobase with a fifth nucleobase that is complementary to the fourth nucleobase, thereby generating an intended edited base pair (e.g., C:G to T:A). In some embodiments, the fifth nucleobase is a thymine. In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the base editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the adenine base editors provided herein. In some embodiments, a target window is a deamination window.

In some aspects, the disclosure provides improved adenine base editors with expanded target windows. In some embodiments, the target window of the disclosed base editors corresponds to protospacer positions 3-8 of the target sequence, wherein protospacer position 0 corresponds to the position of the first contiguous nucleotide of the guide RNA sequence that is complementary to the target sequence, or to the position of the transcription start site of the target gene. In some embodiments, the base editors with wider target windows comprise TadCBEa (set forth in SEQ ID NO: 19). In some embodiments, the base editors with wider target windows comprise TadCBEb (SEQ ID NO: 20). In some embodiments, the base editors with wider target windows comprise TadCBEc (SEQ ID NO: 21). Protospacer position 0 may also refer to the nucleotide position most distal from the PAM. In other embodiments of the disclosed base editors, the base editors have an expanded target window that corresponds to protospacer positions 3-14 of the target sequence relative to the position of the transcription start site of the target gene. In other embodiments, the target window corresponds to protospacer positions 4-11. In still other embodiments, the target window corresponds to protospacer positions 8-14. In still other embodiments, the target window corresponds to protospacer positions 9-14. In some embodiments, the target window is in a gene (e.g. HBG, HBB, or BC11A).

In some embodiments, the target DNA sequence comprises a sequence associated with a disease or disorder. In some embodiments, the target DNA sequence comprises a point mutation associated with a disease or disorder. In some embodiments, the activity of the base editor (e.g., comprising an evolved cytidine deaminase and a Cas9 domain), or the complex, results in a correction of the point mutation. In some embodiments, the target DNA sequence comprises a C→T point mutation associated with a disease or disorder, and wherein the deamination of the mutant C base results in a sequence that is not associated with a disease or disorder. In some embodiments, the target DNA sequence encodes a protein, and the point mutation is in a codon and results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid. In some embodiments, the contacting is in vivo in a subject. In some embodiments, the subject has or has been diagnosed with a disease or disorder.

### Multiplexed Base Editing Applications

In some aspects, the present disclosure provides methods of editing two or more nucleic acid target sites using the disclosed cytosine base editors simultaneously. In multiplexed base editing of unique genomic loci, a plurality of gRNAs having complementarity to different target sequences enables the formation of base editor-gRNA complexes at each of several (e.g. 5, 10, 15, 20, 25, or more) target sequences simultaneously, or within a single iteration or cycle. In some embodiments of the disclosed multiplexed base editing methods, the disclosed TadCBEs can target multiple genes or multiple chromosomes in a human cell, such as a primary human T cell.

The discovery and widespread implementation of the CRISPR/Cas system has dramatically expanded the toolbox for genome engineering and has revolutionized the future prospects of basic biological research, data storage in living systems, agricultural science, and medicine. An advantage of CRISPR/Cas-based genome editors over prior approaches is the capacity to multiplex by using several guide RNAs (gRNAs). This not only enables the screening of libraries of guides in a single cell population but also the targeting of up to six unique loci at once. However, the editing efficiency at each site tends to decrease when compared to that of a single guide transfection.

The present disclosure provides for methods of base editing comprising: contacting a nucleic acid molecule (e.g. DNA) with a plurality of complexes, wherein each complex comprises a base editor and a guide RNA (gRNA) bound to the napDNAbp domain of the base editor, wherein at least two of the complexes of the plurality each comprise a unique gRNA comprising a guide sequence of at least 10 contiguous nucleotides that is complementary to a unique target sequence in the genomic DNA of a cell. In certain embodiments, the cell is a eukaryotic cell, e.g. a mammalian cell. In certain embodiments, the cell is a human cell. In certain embodiments, the plurality of the disclosed base editor-gRNA complexes make simultaneous edits (i.e., within a single iteration) at various target loci within a eukaryotic cell, e.g. a mammalian cell.

In some embodiments, any of the target sequences of these multiplexed editing methods comprises a genomic locus. In some embodiments, the multiple target sequences comprise unique genomic loci. In some embodiments, at least one of the target sequences comprises a sequence in an HBG promoter or the BCL11A enhancer. In some embodiments, at least one of the target sequences comprises a sequence in the CXCR4 or CCR5 genes. Thus, in some embodiments, multiplexed base editing methods are used to install C-to-T mutations simultaneously, or within a single iteration or cycle in the CXCR4 and CCR5 genes (see Example 5).

### Methods of Treatment

The present disclosure provides methods for the treatment of a subject diagnosed with a disease associated with or caused by a point mutation that can be corrected by a fusion protein provided herein (e.g., a base editor fusion protein comprising any of the Nme2Cas9 variants described herein, and a deaminase). For example, in some embodiments, a method is provided that comprises administering to a subject having such a disease, e.g., a disease such as cancer associated with a point mutation, an effective amount of a base editor, and a gRNA that forms a complex with the base editor, that corrects the point mutation or introduces a deactivating mutation into a disease-associated gene. In some embodiments, a method is provided that comprises administering to a subject having such a disease, e.g., a cancer associated with a point mutation, an effective amount of a base editor-gRNA complex that corrects the point mutation or introduces a deactivating mutation into a disease-associated gene. Further provided herein are methods comprising administering to a subject one or more vectors that contains a nucleotide sequence that expresses the base editor and gRNA that forms a complex with the base editor.

In some embodiments, the disease is a proliferative disease. In some embodiments, the disease is a genetic disease. In some embodiments, the disease is a neoplastic disease. In some embodiments, the disease is a metabolic disease. Other diseases that can be treated by correcting a point mutation or introducing a deactivating mutation into a disease-associated gene will be known to those of skill in the art, and the disclosure is not limited in this respect.

The present disclosure provides methods for the treatment of additional diseases or disorders, e.g., diseases or disorders that are associated or caused by a point mutation that can be corrected by base editing. Some such diseases are described herein, and additional suitable diseases that can be treated with the strategies and fusion proteins (e.g., base editors) provided herein will be apparent to those of skill in the art based on the present disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation, sickle cell disease, progeria, cystic fibrosis, and ornithine transcarbamylase (OTC) deficiency. In some embodiments, the disclosed compositions and methods may be suitable for editing a clinically relevant point mutation in sickle cell disease, such as HBB^{S}, the Makassar allele.

Exemplary methods for the treatment of diseases, disorders or conditions using one or more cytidine or adenine base editors by correcting a point mutation or introducing a deactivating mutation into a disease-associated gene are disclosed in International Publication Nos. WO 2021/222318, published Nov. 4, 2021; WO 2021/183693, published Sep. 16, 2021; WO 2021/158999, published Aug. 12, 2021; WO 2020/051360, published Mar. 12, 2020; and WO 2019/079347, published Apr. 25, 2019, each of which is herein incorporated by reference.

In some aspects, the present disclosure provides uses of any one of the fusion proteins (e.g., base editors) described herein, and a guide RNA targeting this base editor to a target C:G base pair in a nucleic acid molecule, in the manufacture of a kit for base editing, wherein the base editing comprises contacting the nucleic acid molecule with the base editor and guide RNA under conditions suitable for the substitution of the cytosine (C) of the C:G nucleobase pair with a thymine (T). In some embodiments of these uses, the nucleic acid molecule is a double-stranded DNA molecule. In some embodiments, the step of contacting induces separation of the double-stranded DNA at a target region. In some embodiments, the step of contacting further comprises nicking one strand of the double-stranded DNA, wherein the one strand comprises an unmutated strand that comprises the G of the target C:G nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the fusion proteins described herein as a medicament. The present disclosure also provides uses of any one of the complexes of fusion proteins and guide RNAs described herein as a medicament.

### Pharmaceutical Compositions

Some aspects of the present disclosure relate to pharmaceutical compositions comprising any of the adenosine-to-cytidine deaminases, base editors, or the base editor-gRNA complexes described herein. Still other aspects of the present disclosure relate to pharmaceutical compositions comprising any of the polynucleotides or vectors that comprise a nucleic acid segment that encodes the TadA-CD deaminases, base editors, or the base editor-gRNA complexes described herein. The disclosure further provides pharmaceutical compositions that comprise particles comprising the rAAV vectors, dual rAAV vectors and ribonucleoproteins described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

In some embodiments, any of the base editors, gRNAs, and/or complexes described herein are provided as part of a pharmaceutical composition. In some embodiments, the pharmaceutical composition comprises any of the base editors provided herein. In some embodiments, the pharmaceutical composition comprises any of the complexes provided herein. In some embodiments pharmaceutical composition comprises a gRNA, a base editor, and a pharmaceutically acceptable excipient. Pharmaceutical compositions may optionally comprise one or more additional therapeutically active substances.

In some embodiments, compositions provided herein are formulated for delivery to a subject, for example, to a human subject, in order to affect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been affected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; 7,163,824, 9,526,784; 9,737,604; and U.S. Patent Publication Nos. 2018/0127780, published May 10, 2018, and 2018/0236081, published Aug. 23, 2018, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131, filed Nov. 2, 2010 (Publication No. WO 2011/053982, published May 5, 2011), incorporated in its entirety herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some aspects, the disclosure provides pharmaceutical compositions comprising a plurality of any of the base editors described herein and a gRNA, wherein at least five of the base editors of the plurality are each bound to a unique gRNA, and a pharmaceutically acceptable excipient.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref Biomed. Eng. 14:201; Buchwald et al., 1980, Surgery 88:507; Saudek et al., 1989, N. Engl. J. Med. 321:574). In another embodiment, polymeric materials may be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition may be contained within a lipid particle or vesicle, such as a lipid nanoparticle (LNP), liposome or microcrystal, which is also suitable for parenteral administration. The particles may be of any suitable structure, such as unilamellar or plurilamellar, so long as compositions are contained therein. Compounds may be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition may be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent may be used for reconstitution or dilution of the lyophilized compound of the invention. Optionally associated with such container(s) may be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

The present disclosure provides methods for delivering a cytosine base editor described herein (e.g., in the form of an evolved base editor as described herein, or a vector or construct encoding the same) into a cell. Such methods may involve transducing (e.g., via transfection) cells with a plurality of complexes each comprising a base editor and a gRNA molecule. In some embodiments, the gRNA is bound to the napDNAbp domain (e.g., nCas9 domain) of the base editor. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence. In certain embodiments, the methods involve the transfection of nucleic acid constructs (e.g., plasmids and mRNA constructs or recombinant mRNA constructs) that each (or together) encode the components of a complex of base editor and gRNA molecule. In certain embodiments, any of the disclosed base editors and a gRNA are administered as a protein:RNA complex, such as a ribonucleoprotein (RNP) complex. In some embodiments, any of the disclosed base editors are administered as an mRNA construct, along with the gRNA molecule. In some embodiments, administration to cells is achieved by electroporation or lipofection (e.g., using Lipofectamine®).

In certain embodiments of the disclosed methods, a nucleic acid construct (e.g., an mRNA construct) that encodes the base editor is transfected into the cell separately from the construct that encodes the gRNA molecule. In certain embodiments, these components are encoded on a single construct and transfected together. In other embodiments, the methods disclosed herein involve the introduction into cells, in vivo or in vitro, of a complex comprising a base editor and gRNA molecule that has been expressed and cloned outside of these cells. In some embodiments, the disclosed methods involve the introduction of a DNA construct encoding the base editor in an amount of 100 ng.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

In another aspect, the disclosure discloses a pharmaceutical composition comprising any one of the presently disclosed vectors. In certain embodiments, the pharmaceutical composition further comprises a pharmaceutically acceptable excipient. In certain embodiments, the pharmaceutical composition further comprises a lipid and/or polymer. In certain embodiments, the lipid and/or polymer is cationic. The preparation of such lipid particles is well known. See, e.g. U.S. Pat. Nos. 4,880,635; 4,906,477; 4,911,928; 4,917,951; 4,920,016; 4,921,757; and 9,737,604, each of which is incorporated herein by reference.

Exemplary methods of delivery of nucleic acids include lipofection, nucleofection, electroporation (e.g., MaxCyte electroporation), stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., TransfectamT™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g. in vitro or ex vivo administration) or target tissues (e.g. in vivo administration).

In certain embodiments of the disclosed methods, the constructs that encode the base editors are transfected into the cell separately from the constructs that encode the gRNAs. In certain embodiments, these components are encoded on a single construct and transfected together. In particular embodiments, these single constructs encoding the base editors and gRNAs may be transfected into the cell iteratively, with each iteration associated with a subset of target sequences. In particular embodiments, these single constructs may be transfected into the cell over a period of days. In other embodiments, they may be transfected into the cell over a period of hours. In other embodiments, they may be transected into the cell over a period of weeks.

In the disclosed methods, target cells may be incubated with the base editor-gRNA complexes for two days, or 48 hours, after transfection to achieve multiplexed base editing. Target cells may be incubated for 30 hours, 40 hours, 54 hours, 60 hours, or 72 hours after transfection. Target cells may be incubated with the base editor-gRNA complexes for four days, five days, seven days, nine days, eleven days, or thirteen days or more after transfection.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of base editors markedly increases the DNA specificity of base editing. RNP delivery of base editors leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein. In some embodiments, the RNP complex is delivered in a DNA-free engineered virus-like particle (eVLP), which efficiently package and deliver base editor RNPs. See Banskota et al., Cell 185, 250-265, January 2022, which is herein incorporated by reference.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003/0087817, published May 8, 2003, International Patent Application No. WO 2016/205764, published Dec. 22, 2016, International Patent Application No. WO 2018/071868, published Apr. 19, 2018, U.S. Patent Publication No. 2018/0127780, published May 10, 2018, and International Publication No. WO2020/236982, published Nov. 26, 2020, the disclosures of each of which are incorporated herein by reference.

Delivery of Base Editors Using rAAV Vectors

The TadCBEs of the disclosure contain an evolved cytidine deaminase domain containing a single deaminase, i.e. a deaminase monomer (such as a TadA-CDa monomer) i.e. In some embodiments, the TadA-CD monomers are about 166 amino acids in length.

In some embodiments, any of the disclosed size-reduced TadA-CD variants (e.g., TadA-CDa, TadA-CDb, TadA-CDc, and TadA-CDd, are compatible with single-AAV delivery as described in Davis et al., Nat Biomed Eng. 2022 Jul. 28, which is incorporated herein by reference. Each contain the TadA-CD adenosine-to-cytidine deaminase and the nickase variant of SauriCas9, SaKKH-Cas9, and SaCas9, respectively. In any of these disclosed base editors, the wild-type, or the nickase variant, of SauriCas9, SaKKH-Cas9, SaCas9, CjCas9, and Nme2Cas9, respectively, may be used.

Aspects of the presently disclosed delivery methods relate to using recombinant adeno-associated virus vectors for the delivery of any of the disclosed nucleic acid molecules. The rAAV particles of the present disclosure comprise a rAAV vector (i.e., a recombinant genome of the rAAV) encapsidated in the viral capsid proteins. See U.S. Patent Publication No. 2018/0127780, published May 10, 2018, and PCT Publication No. WO 2020/236982, published Nov. 26, 2020, the disclosures of each of which are incorporated herein by reference.

In some embodiments, the AAV nucleic acid vector is single-stranded. In some embodiments, the AAV nucleic acid vector is self-complementary. In various embodiments, the rAAV vectors of the disclosure do not contain any inteins.

In some embodiments, viral sequences that facilitate integration comprise Inverted Terminal Repeat (ITR) sequences. In some embodiments, nucleic acid molecule is flanked on each side by an ITR sequence. In some embodiments, the nucleic acid vector further comprises a region encoding an AAV Rep protein as described herein, either contained within the region flanked by ITRs or outside the region. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV8 or AAV9. In some embodiments, in methods of packaging any of the disclosed rAAV particles, a nucleic acid plasmid, such as a helper plasmid, that comprises a region encoding a Rep protein and/or a Cap (capsid) protein is provided.

In various embodiments, any of the disclosed base editor (or fusion protein) constructs may be engineered for delivery in one or more AAV vectors. Any of the disclosed AAV vectors may comprise 5′ and 3′ inverted terminal repeats (ITRs) that flank the polynucleotide (or construct) encoding any of the disclosed base editors. In some embodiments, any of the base editor constructs may be engineered for delivery in a single rAAV vector. In some embodiments, any of the disclosed base editor constructs has a length of 4.9 kilobases or less, and as such may be packaged into a single AAV vector, while being flanked by ITRs. In some embodiments, any of the disclosed base editor constructs has a length of between about 4.65 kb, about 4.70 kb, about 4.725 kb, about 4.75 kb, about 4.80 kb, about 4.825 kb, about 4.85 kb, or about 4.90 kb between the 5′ and 3′ ITRs. In some embodiments, any of the disclosed base editor constructs has a length of between 4.7 kb and 4.9 kb, such as about 4.8 kb.

In some embodiments, any of the disclosed base editor constructs or rAAV vectors containing a polynucleotide encoding a base editor comprises a first segment encoding the base editor, and further comprises a second nucleic acid segment encoding a guide RNA, such as a single-guide RNA. In some embodiments, the orientation of this gRNA-encoding (second) nucleic acid segment is reversed relative to the orientation of the segment encoding the base editor. In some embodiments, the first nucleic acid segment is operably controlled by a first promoter, and the second nucleic acid segment is operably controlled by a second promoter (e.g., a U6 promoter). In several embodiments, the first promoter is different from the second promoter. The disclosure provides single AAV vectors comprising any of the above-contemplated base editor constructs.

The disclosure provides recombinant AAV particles comprising any of the disclosed AAV vectors. These rAAV particles may comprise an AAV vector and a capsid protein. The capsid protein may be of any serotype.

Accordingly, an rAAV particle as related to any of the disclosed uses, methods, and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole base editor that is carried by the rAAV into a cell) that is to be delivered to a cell. An rAAV may be chimeric.

Any of the disclosed base editors may be delivered by a single AAV vector. In some embodiments, the AAV vector comprise size-minimized base editors and regulatory components that enable the vector to have a length within the 4.7 kb-4.9 kb packaging capacity of a single AAV vector. In some embodiments, the single AAV vector contains a first nucleic acid segment comprising: (i) a 5′ ITR; (ii) a first nucleic acid segment comprising sequence encoding a base editor operably linked to a first promoter, wherein the base editor comprises a nucleic acid programmable DNA binding protein (napDNAbp) domain and a deaminase domain; and a polyadenylation (polyA) signal; (iii) a second nucleic acid segment encoding a guide RNA (gRNA) operably linked to a second promoter; and (iv) a 3′ ITR, wherein the length between the 5′ ITR and the 3′ ITR is less than about 4.90 kb. In some embodiments, the rAAV vectors consist essentially of components (i)-(iv). In some embodiments, the base editor delivered by a single AAV vector contains a napDNAbp domain that is a compact protein, such as an S. aureus Cas9 (SaCas9), an N. meningitidis 2 Cas9 (Nme2Cas9), a C. jejuni Cas9 (CjCas9), or an S. auricularis (SauriCas9) domain, or a variant thereof.

Some aspects of the disclosed delivery methods entail encoding the editor, and further encoding a guide RNA, in a single AAV vector for packaging in a single rAAV particle. Accordingly, in some embodiments, any of the disclosed base editors may be encoded in a single AAV vector, without the use of any split points or inteins. Several other special considerations to account for the unique features of base editing are described, including the optimization of second-site nicking targets and properly packaging base editors into virus vectors, including lentiviruses and rAAV.

Accordingly, the disclosure provides rAAV vectors and rAAV vector particles that comprise expression constructs that encode any of the disclosed base editors. In exemplary embodiments, any of the disclosed base editors are delivered to one or more cells in a single rAAV particle.

In some aspects, the disclosure provides compositions containing a plurality of any of the disclosed rAAV particles. In some aspects, the disclosure provides host cells containing a plurality of any of the disclosed rAAV particles. In some embodiments, the host cells are mammalian cells, such as human cells or rodent cells. In exemplary embodiments, the host cells are human cells. In other embodiments, the host cells are yeast cells, plant cells, or bacterial cells.

In other embodiments, the base editors may be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their trans-splicing inside the cell and the concomitant restoration of the complete, functioning TadCBE. These split intein-based methods may overcome several barriers to in vivo delivery. For example, the DNA encoding some base editors is larger than the recombinant AAV (rAAV) packaging limit, and so requires different solutions. One such solution is formulating the editor fused to split intein pairs that are packaged into two separate rAAV particles that, when co-delivered to a cell, reconstitute the functional editor protein. In such embodiments, the base editor may be divided into two halves at a split site. These two halves may be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their trans-splicing inside the cell and the concomitant restoration of the complete, functioning TadCBE.

Methods of delivery to a target cell or target tissue of any of the disclosed rAAV particles and compositions and host cells comprising rAAV particles are known in the art. In some embodiments, any of the disclosed rAAV particles, host cells, or compositions are delivered to a subject, such as a mammalian subject. In some embodiments, the rAAV particles are delivered to a human subject.

In some embodiments, the disclosed rAAV particles and compositions are administered to a subject in a single injection, such as a single systemic injection. In some embodiments, the disclosed rAAV particles and compositions are administered to a subject in multiple injections. rAAV particles are known to transduce target tissues within days, but are typically allowed three to four weeks to complete transduction, genome integration, and clearance, from the cell. Accordingly, in some aspects, any of the disclosed rAAV particles or compositions are administered to a subject for a period of three weeks. in some aspects, any of the disclosed rAAV particles or compositions are administered to a subject for a period of between three and four weeks.

In some embodiments, any of the disclosed rAAV particles or compositions is administered to a subject or a target tissue in a therapeutically effective amount of about 10¹⁵, about 10¹⁴, about 10¹³, about 10¹², about 10¹¹, or less than about 10¹¹ vector genomes (vg) per kg weight of the subject. In some embodiments, the rAAV particles are administered in an amount of between 10¹⁵ and 10¹⁴, between 10¹⁴ and 10¹³, between 10¹³ and 10¹², between 10¹² and 10¹¹, or between 10¹² and 10¹¹ vgs per kg. In some embodiments, the rAAV particles are administered in an amount of between 10¹⁴ and 10¹¹ vgs per kg. In some embodiments, any of the disclosed rAAV particles or compositions is administered to a target tissue of a subject in a lower dose than is convention for dual AAV particle delivery, such as that described in PCT Publication No. WO 2020/236982, published Nov. 26, 2020 and Levy, J. M., et al. Nat Biomed Eng 4, 97-110 (2020).

As used herein, the serotype of an rAAV particle refers to the serotype of the capsid protein of the recombinant virus. In some embodiments, the rAAV particles disclosed herein comprise an rAAV2, rAAV3, rAAV3B, rAAV4, rAAV5, rAAV6, rAAV8, rAAV9, rAAV10, rPHP.B, rPHP.eB, or rAAV9 particle, or a variant thereof. In particular embodiments, the disclosed rAAV particles are rAAV8 or rAAV9 particles.

Non-limiting examples of serotype derivatives and pseudotypes include rAAV2/1, rAAV2/5, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol. Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the rAAV vector of the present disclosure comprises one or more regulatory elements to control the expression of the heterologous nucleic acid region (e.g., promoters, transcriptional terminators, and/or other regulatory elements). In some embodiments, the first and/or second nucleotide sequence is operably linked to one or more (e.g., 1, 2, 3, 4, 5, or more) transcriptional terminators. Non-limiting examples of transcriptional terminators that may be used in accordance with the present disclosure include transcription terminators (or polyadenylation signals) of the bovine growth hormone gene (bGH), human growth hormone gene (hGH), SV40, CW3, ϕ, or combinations thereof. In exemplary embodiments, the transcriptional terminator is an SV40 polyadenylation signal. In exemplary embodiments, the transcriptional terminator does not contain a posttranscription response element, such as WPRE element.

In some aspects, provided herein are methods of making (or manufacturing, or packaging) any of the disclosed rAAV particles. rAAV particles may be manufactured according to any method known in the art. Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US 2007-0015238 and US 2012-0322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into recombinant cells such that the rAAV particle can be packaged and subsequently purified.

In some embodiments, the disclosed rAAV particles provide for transduction of the target tissue to achieve expression and translation of the payload or transgene, e.g., a base editor in accordance with the present disclosure, for a sufficient duration to install desired mutations in the genome of a target cell. In some embodiments, the desired mutation is a C to T mutation. In some embodiments, the disclosed rAAV particles provide for sufficient expression and translation of the base editor transgene for a sufficient duration to install desired (on-target) mutations in the genome with a tolerable degree of off-target effects, such as bystander edits. In some embodiments, the disclosed rAAV particles provide for sufficient expression and translation of the base editor transgene for a sufficient duration to install desired mutations in the genome without appreciable off-target editing. In some embodiments, the disclosed rAAV particles provide for sufficient expression and translation of the base editor transgene for a sufficient duration to install desired mutations in the genome without appreciable bystander editing.

Suitable routes of administrating the disclosed compositions of rAAV particles include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, systemic, intravascular, intraosseus, periocular, intratumoral, intracerebral, parenteral, and intracerebroventricular administration. In some embodiments, the route of administration is systemic (intravenous). In some embodiments, the pharmaceutical composition described herein is administered locally to a diseased site.

Additional methods for the delivery of nucleic acids to cells are known to those skilled in the art. See, for example, US Pub. No. 2003/0087817, incorporated herein by reference. It should be appreciated that any base editor, e.g., any of the base editors provided herein, may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a virus encoding a base editor), or transfected (e.g., with a plasmid encoding a base editor) with a nucleic acid that encodes a base editor, or the translated base editor. Such transduction may be a stable or transient transduction. In some embodiments, cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into cells through electroporation, transient (e.g., lipofection) and stable genome integration (e.g., piggybac) and viral transduction or other methods known to those of skill in the art.

### Kits and Cells

Some aspects of this disclosure provide kits comprising a nucleic acid construct comprising a nucleotide sequence encoding an adenosine deaminase capable of deaminating an adenosine in a deoxyribonucleic acid (DNA) molecule. In some embodiments, the nucleotide sequence encodes any of the adenosine deaminases provided herein. In some embodiments, the nucleotide sequence comprises a heterologous promoter that drives expression of the adenosine deaminase. The nucleotide sequence may further comprise a heterologous promoter that drives expression of the gRNA, or a heterologous promoter that drives expression of the base editor and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising a nucleic acid construct, comprising (a) a nucleotide sequence encoding a napDNAbp (e.g., a Cas9 domain) fused to an adenosine deaminase, or a base editor comprising a napDNAbp (e.g., Cas9 domain) and an adenosine deaminase as provided herein; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

In certain embodiments, the kit comprises (a) a nucleic acid sequence encoding any one of the base editors of the current invention, (b) a nucleic acid sequence encoding a gRNA, and one or more heterologous promoters that drive the expression of the sequence of (a) and/or the sequence of (b). In some cases, the kit further comprises an expression construct encoding a guide RNA backbone and a cloning site positioned to allow the cloning of a nucleic acid sequence identical or complementary to a target sequence into the guide RNA backbone.

Some embodiments of this disclosure provide host cells comprising any of the base editors or complexes provided herein. In some embodiments, the host cells comprise nucleotide constructs that encodes any of the base editors provided herein. In some embodiments, the cells comprise any of the nucleotides or vectors provided herein. In some embodiments, the cell is a stem cell. In some embodiments, the cell is a human stem cell, such as a human stem and progenitor cell (HSPC). In some embodiments, the cell is a mobilized (e.g., plerixafor-mobilized) peripheral blood HSPC. In certain embodiments, the cell is a T cell, such as a primary human T cell. In some cases, the cells is a human HSC.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. In some embodiments, the cell has been removed from a subject and contacted ex vivo with any of the disclosed base editors, complexes, vectors, or polynucleotides.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, the host cell is a cell that has been removed from a subject and contacted ex vivo with any of the base editors, complexes, or vectors described herein.

In some aspects, the present disclosure provides uses of any one of the base editors described herein and a guide RNA targeting this base editor to a target A:T base pair in a nucleic acid molecule in the manufacture of a kit for nucleic acid editing, wherein the nucleic acid editing comprises contacting the nucleic acid molecule with the base editor and guide RNA under conditions suitable for the substitution of the adenine (A) of the A:T nucleobase pair with an guanine (G). In some embodiments of these uses, the nucleic acid molecule is a double-stranded DNA molecule. In some embodiments, the step of contacting of induces separation of the double-stranded DNA at a target region. In some embodiments, the step of contacting thereby comprises nicking one strand of the double-stranded DNA, wherein the one strand comprises an unmutated strand that comprises the T of the target A:T nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the adenine base editors described herein as a medicament. The present disclosure also provides uses of any one of the complexes of adenine base editors and guide RNAs described herein as a medicament.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### EXAMPLES

### Example 1. Development of a Phage-Assisted Evolution Selection for Cytidine Deamination

Phage-assisted continuous evolution (PACE) has enabled the rapid laboratory evolution of diverse protein functions including protein-protein interactions³⁵, tRNA synthetases³⁶, DNA-binding proteins³⁷⁻³⁹, proteases^{40,41}, polymerases⁴², metabolic enzymes⁴³⁻⁴⁵, and base editors^{9,12}. During PACE, the evolving protein is encoded on the selection phage (SP), which infect E. coli host cells⁴⁶. The E. coli harbor a mutagenesis plasmid (MP) that constantly mutagenizes the phage genome, as well as accessory plasmid(s) (AP) that establish a selection circuit that regulates the expression of gene III, which encodes pIII, a critical protein for phage replication. Since gIII has been removed from the SP genome, only phage that encode evolving variants with the desired activity trigger the production of pIII in E. coli and replicate, resulting in the propagation of active gene variants (FIG. 1B). Under constant mutagenesis and dilution, phage lacking the desired activity are rapidly diluted from the selection vessel (“lagoon”), while phage that evolve beneficial mutations persist.

Previously, a CBE-PACE selection¹² was developed in which a cytidine deaminase is encoded within the SP and host E. coli cells contain (i) the MP, (ii) an accessory plasmid that encodes SpCas9, (iii) a self-inactivating T7 RNA polymerase (T7 RNAP) fused to a C-terminal degron, and (iv) gene III under T7 RNAP transcriptional control. Upon phage infection, the SP-encoded deaminase is joined to Cas9 by trans-intein splicing to reconstitute the base editor. To activate the selection circuit, the base editor must perform C·G-to-T·A editing to create a stop codon between T7 RNAP and the degron, yielding active T7 RNAP. Degron-free T7 RNAP then transcribes gIII, leading to phage propagation¹².

To develop a PACE circuit to select for cytidine deamination by TadA, the previous CBE selection circuit was modified to accommodate an enzyme with high initial adenosine deamination activity (FIG. 1C). In the original circuit, TGG (Trp) was edited into a stop codon (TAG, TGA, or TAA) through C-to-T conversion of CCA in the template strand. This strategy, however, placed adenine, which was opposite thymine in all stop codons (TAG, TGA, TAA), at position 6 within the target protospacer. Given that position 6 is highly edited by ABE8e⁹, and that A-to-G editing of A₆ precludes stop codon formation since CGG, CAG, CGA, and CAA all encode amino acids, this original circuit required high selectivity for cytidine over adenosine deamination that was unlikely to be found among early-stage evolved ABE8e variants.

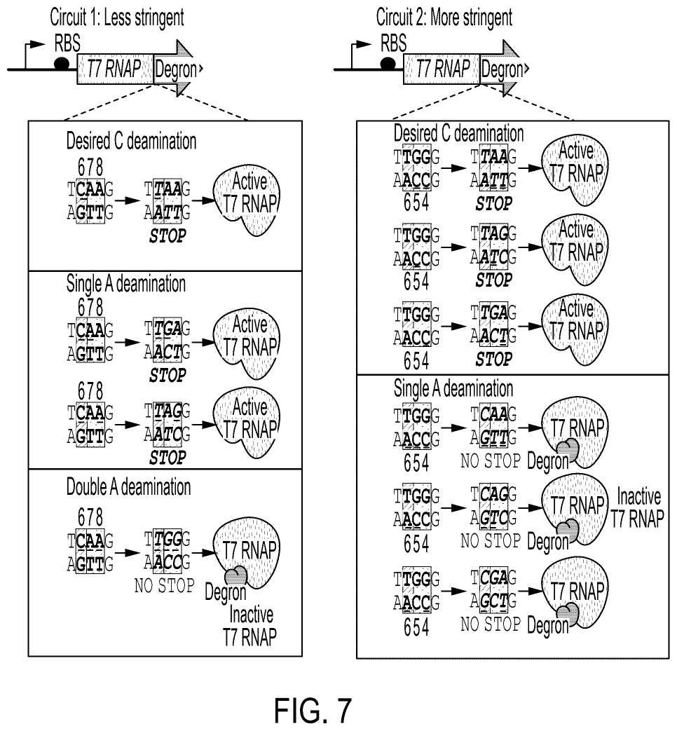

To address this problem, a new selection circuit that instead edited the non-template strand was developed (FIG. 1D). In the new circuit, C₆A₇A₈, was edited to T₆A₇A₈ to introduce a stop codon upon cytidine deamination. Adenosine deamination does not prevent stop codon installation (TAA, TGA, or TAG) in the new selection unless both A₇ and A₈ are converted to Gs (TGG=Trp), making this circuit tolerant to modest levels of adenosine deamination and thus more suitable for early-stage TadA8e evolution (Circuit 1). Following the initial evolution in the new circuit, it was possible to switch to the original template-strand circuit (Circuit 2) to take advantage of its inherent strong negative selection against adenosine deamination (FIG. 7).

### Example 2. Cytidine Deaminase Evolution

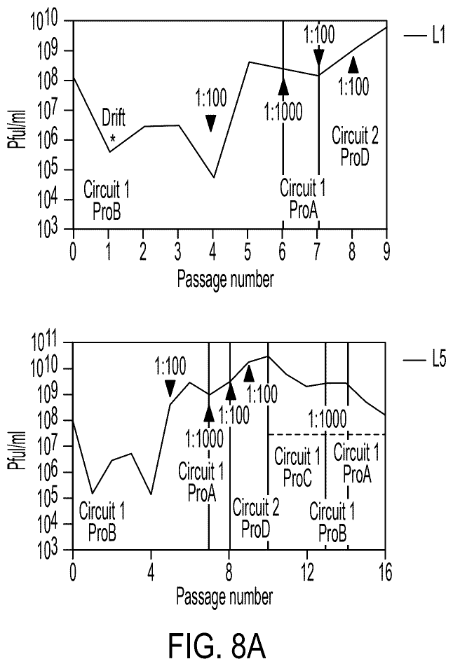

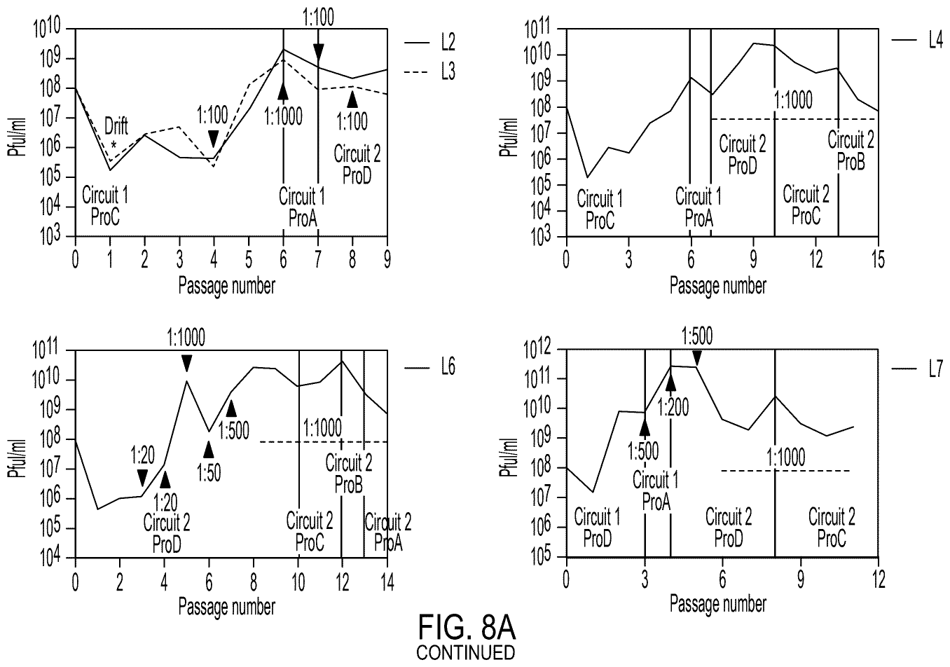

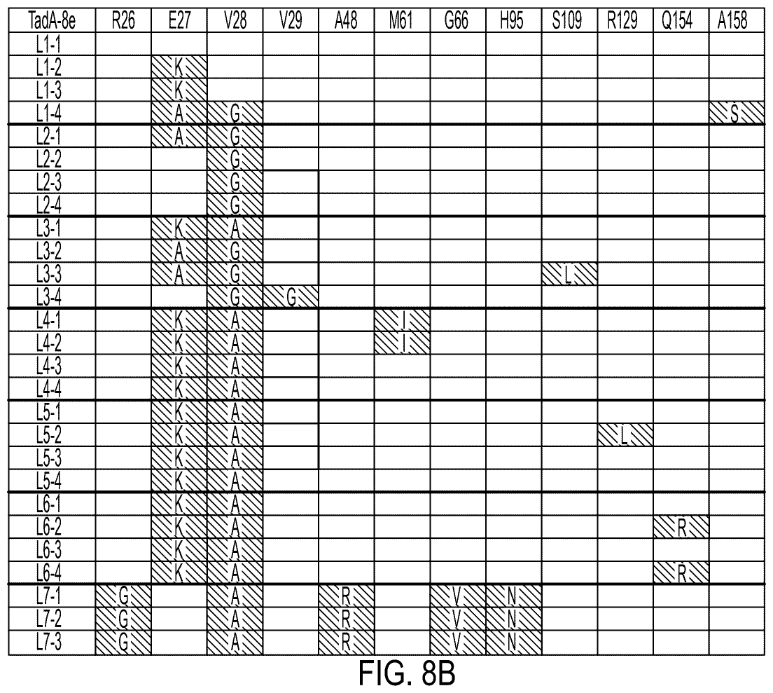

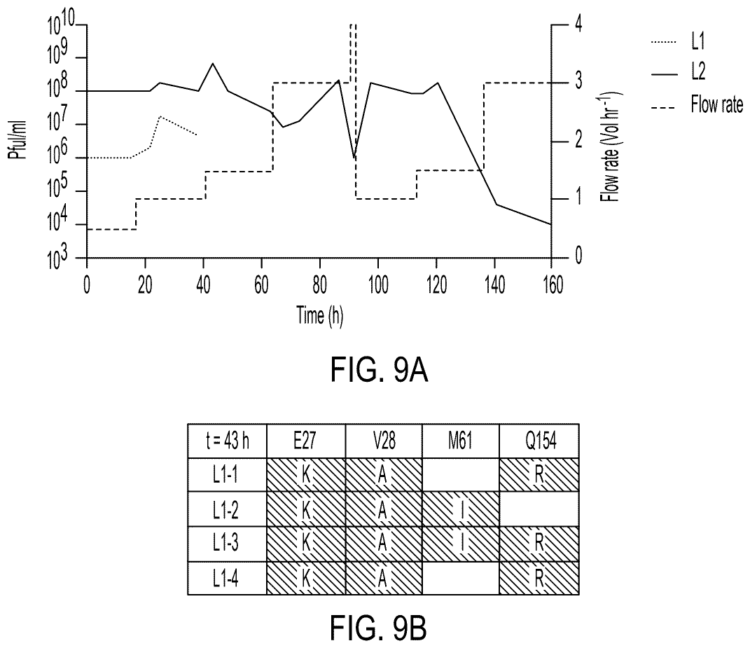

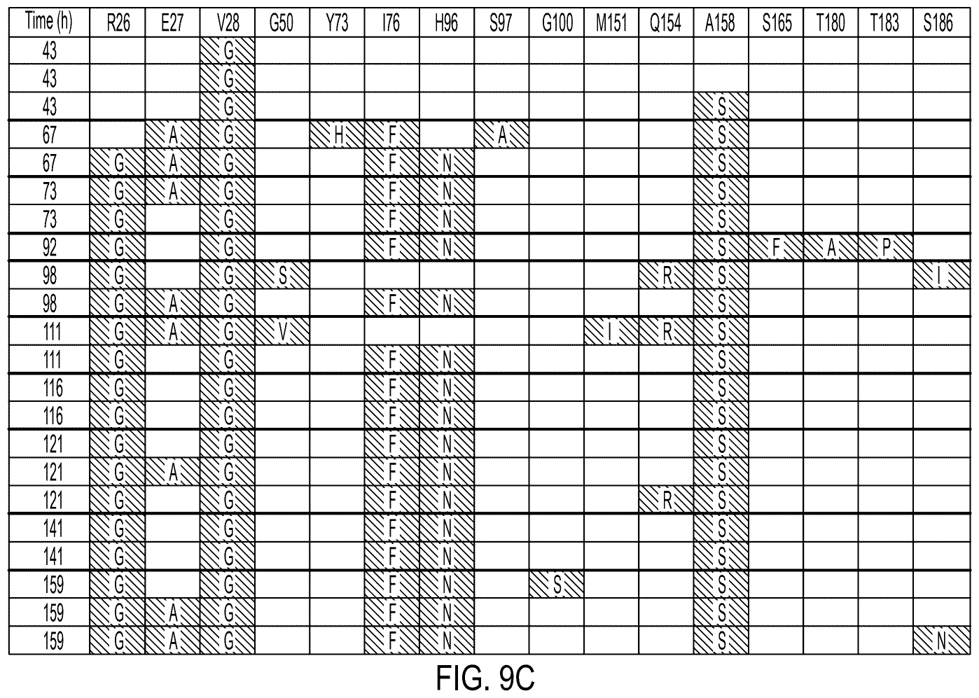

A phage-assisted non-continuous evolution (PANCE) of TadA-8e using Circuit 1 was initiated (FIG. 1E). In PANCE, E. coli host cells containing the AP and MP were infected with phage containing the gene of interest and grown overnight, without continuous dilution. The next day, the supernatant containing the phage were diluted into a fresh host cell culture and the process was repeated to enrich for phage harboring active cytidine deaminases. Compared to PACE, PANCE offers lower stringency and thus is helpful during early-phase evolution campaigns in which preserving genetically diverse variants with low initial activity can be critical^{9,41,43}. Following four rounds of PANCE with induced MP6 mutagenesis⁴⁷, the phage began to propagate >100-fold overnight, suggesting improved activity for cytidine deamination. To increase the stringency of the selection, the fold-dilution between passages was increased, and the strength of the promoter upstream of T7 RNAP was decreased (Table 1, FIGS. 8A-8C). Next, Circuit 2 was used for additional passages of PANCE (FIGS. 8A-8C) to select against adenosine deamination while maintaining cytidine deamination activity. To further increase selection stringency, 159 hours of continuous evolution (PACE) was performed on phage pools surviving PANCE using Circuit 2 (FIGS. 9A-9C). TadA-8e variants emerging from all phases of PANCE and PACE survived an average total dilution of ˜10¹³⁹-fold.

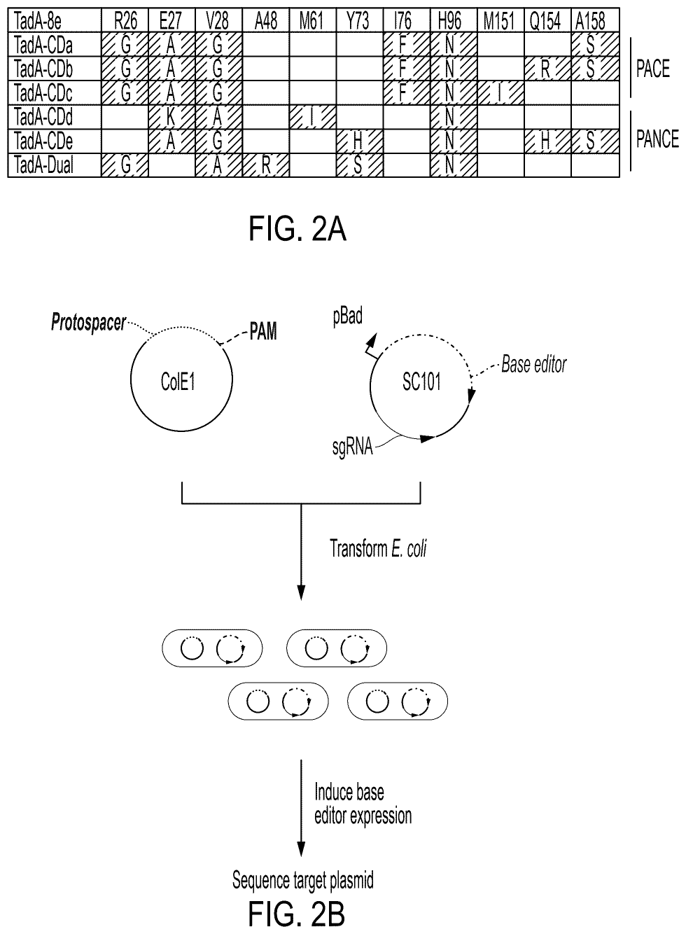

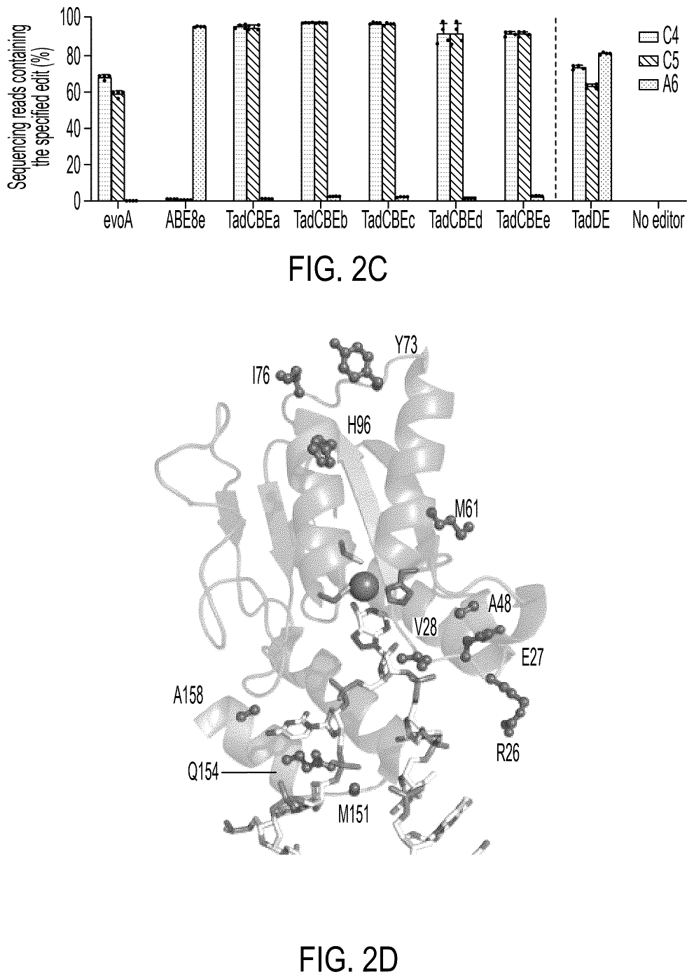

Individual phages surviving PANCE and PACE were isolated and sequenced to identify TadA-8e mutations acquired during evolution (FIG. 2A, FIGS. 8A-9C). A striking prevalence of mutations in residues 26-28 were observed across all the sequenced phages, with R26G, E27K, E27A, and V28G mutations highly represented across several separately evolved lagoons. Next, the evolved variants were assayed for base editing in E. coli. Three evolved TadA variants from phage were subcloned from phage into the BE4max architecture⁴⁸ (from N-terminus to C-terminus: TadA*-SpCas9-UGI-UGI) on a low-copy plasmid, and a high-copy target plasmid containing sequences from the selection circuits on which the phage evolved was designed. The base editor plasmid, which also encodes the guide RNA, and target plasmid into E. coli cells, was co-transformed which allowed editing following arabinose induction to occur overnight. Afterwards, high-throughput sequencing of the target plasmid was performed (FIG. 2B).

The sequencing results revealed a striking shift in selectivity of the evolved TadA variants compared to the starting TadA-8e variant. While base editors containing TadA-8e yielded 90% A·T-to-G·C at A₆ and 1-2% C·G-to-T·A editing at C₄ and C₅ in the target plasmid, the evolved variants instead resulted in 85-92% editing of cytosines and 1-2% editing of adenine (FIG. 2C), representing a >3,000-fold change in cytosine versus adenine base editing. These results indicated that PANCE and PACE using selection Circuits 1 and 2 evolved TadA variants, hereafter referred to as TadA-cytidine deaminases (TadA-CDs), with strong cytidine deamination activity and high selectivity for cytosine over adenine base editing.

From a lagoon infected with TadA-8e A48R, containing a mutation that increases promiscuity in TadA-7.10³², a variant that performed both A·T-to-G·C (80%) and C·G-to-T·A editing (73%) was also identified in the E. coli editing assay (FIG. 2C). This variant thus serves as a TadA-based dual editor (TadDE). TadDE is smaller than previously reported dual editors that fuse both cytidine and adenosine deaminases to a Cas domain⁴⁹⁻⁵³, and may be especially useful for applications requiring broad mutagenesis⁵⁴, such as genetic screens^{55,56}

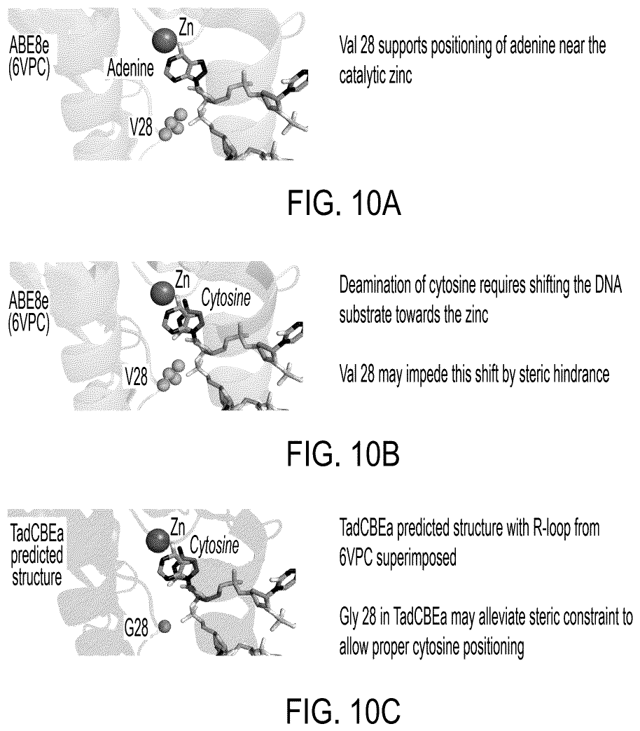

To identify potential roles for the evolved mutations, the mutations were mapped onto the cryo-EM structure of ABE8e (PDB 6VPC)¹⁸. The highly conserved mutations were predicted to localize to a loop near the active site (FIG. 2D). This loop interacts with the backbone of the single-stranded DNA substrate near the target base and supports productive orientation of the base relative to the catalytic zinc ion. Other conserved mutations, including A158S and Q154R, were also mapped to the interface of TadA and the single-stranded DNA substrate. A structural prediction of TadA-CDa using AlphaFold⁵⁷ suggested that the mutations were not predicted to significantly alter the secondary structure of TadA compared to the cryo-EM structure of ABE8e (6VPC, FIGS. 10A-10C). Instead, the observed mutation of residues 26-28 from Arg-Glu-Val to smaller amino acids such as Gly-Ala-Gly during evolution may alleviate the steric clash that otherwise is predicted to block proper positioning of the pyrimidine C₄ for nucleophilic attack and deamination (FIGS. 10A-10C). These observations collectively suggested that the evolved mutations may alter the conformation of the bound DNA substrate to enable efficient cytidine deamination and impede adenosine deamination.

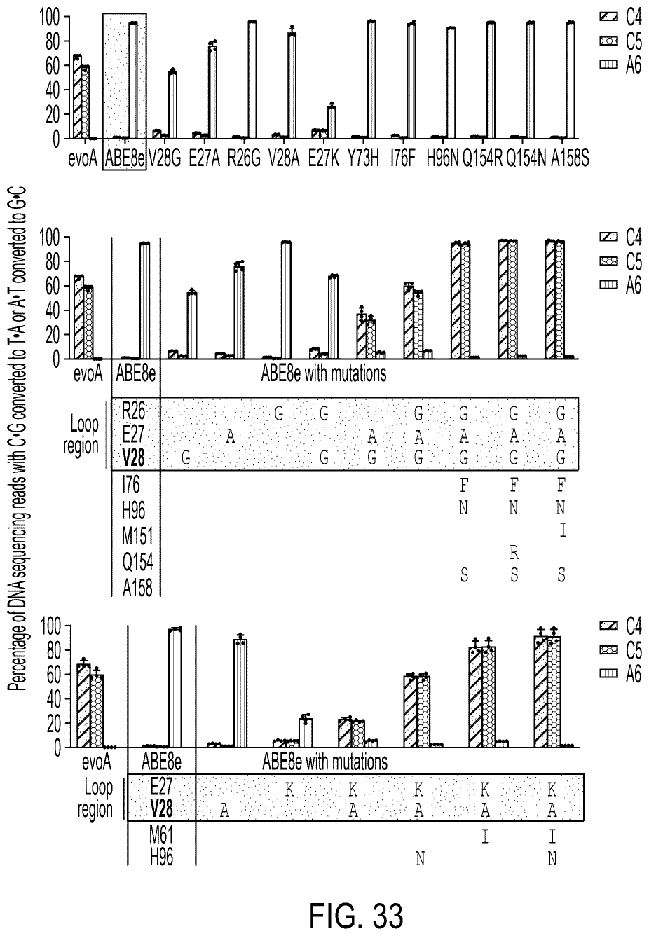

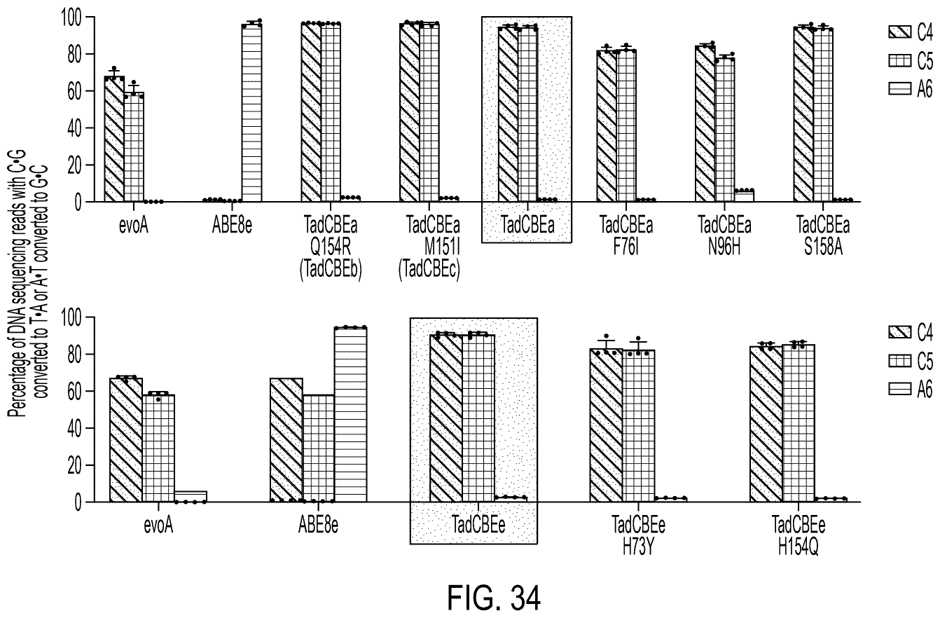

Next, mutagenesis and reversion analysis was performed to interrogate the roles of the mutations found through evolution. In isolation, none of the mutations are sufficient to alter selectivity (FIG. 33). However, the addition of just two mutations to the loop region (E27A V28G in TadCBEa-c,e and E27K V28A in TadCBEd) is sufficient to alter the selectivity of TadCBEs to modestly favor cytidine deamination, albeit with low editing efficiency (FIG. 33). Additional mutations evolved during PANCE or PACE greatly increase activity and improve selectivity for C·G-to-T·A conversion. The reversion of mutations outside of the loop region generally decreases activity, but not selectivity (FIG. 34). This reversion analysis thus supports the significance of residues 26-28 in modulating the deamination selectivity of evolved TadA variants.

### Example 3. Characterization of TadA-CDs in Mammalian Cells, Compatibility of TadCBEs with Cas9 Orthologs, and Editing Windows

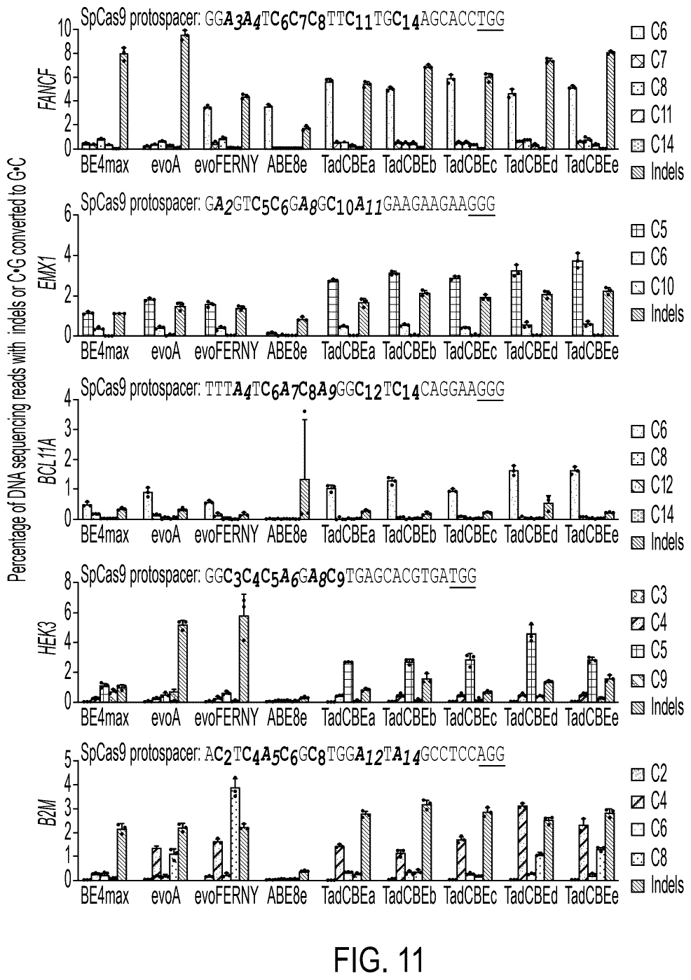

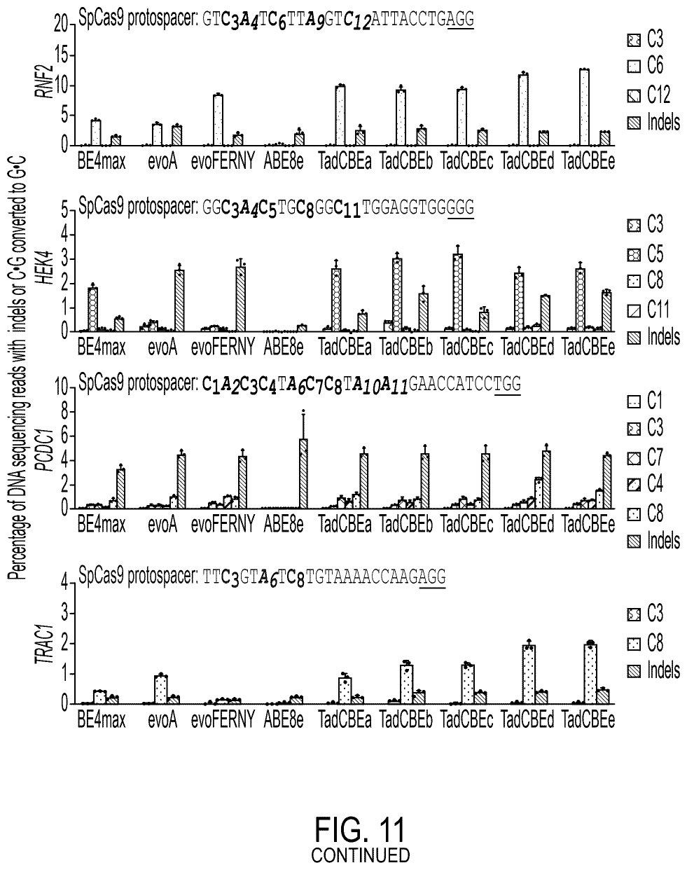

Encouraged by the characteristics of the TadA-CDs in bacteria, the evolved TadA-CD cytosine base editors (TadCBEs) in mammalian cells were evaluated. Five TadCBE variants (TadCBEa-e) were cloned into mammalian expression vectors regulated by a CMV promoter in the BE4max architecture⁴⁸. These five TadCBE variants were assayed alongside three of the most widely used engineered and evolved CBEs: BE4max⁴⁸, evoA¹², and evoFERNY¹². HEK293T cells were co-transfected with each base editor plasmid and an sgRNA plasmid, editing was allowed to occur for 72 hours, and then target sites from genomic DNA were sequenced. Across nine different target sites tested in HEK293T cells, TadCBE variants generally yielded target C·G-to-T·A editing (averaging 51-60% peak editing for TadCBEa-e across all nine tested sites) that were comparable to or higher than that observed from canonical BE4max, evoA, and evoFERNY CBEs (averaging 47%, 55%, and 41% peak editing, respectively, across all nine sites) (FIG. 3 and FIG. 11). These results demonstrated that TadCBEs can perform highly efficient C·G-to-T·A editing in mammalian cells.

Evolved TadCBE variants generally showed low residual A·T-to-G·C editing averaging 1.5-4.5% editing for TadCBEa-e across adenosines in all nine tested sites and thus showed excellent selectivity for C·G-to-T·A editing over A·T-to-G·C editing (FIG. 3). By comparison, ABE8e in the same base editor architecture (with 2×UGI) averaged 31% A·T-to-G·C editing and 2.0% C·G-to-T·A editing across the nine sites. Ratios of desired C·G-to-T·A editing to residual A·T-to-G·C editing for seven of the nine tested sites was very high, averaging 21- to 42-fold for TadCBE variants a, c, d, and e, and 9.2-fold for TadCBEb (FIG. 3). Taken together, these observations suggested that residual A·T-to-G·C editing was generally low among evolved TadCBE variants and limited primarily to a small subset of target sites, protospacer positions, and TadCBE variants. The introduction of V106W in the deaminase domain can further reduce residual A·T-to-G·C editing when necessary (see infra).

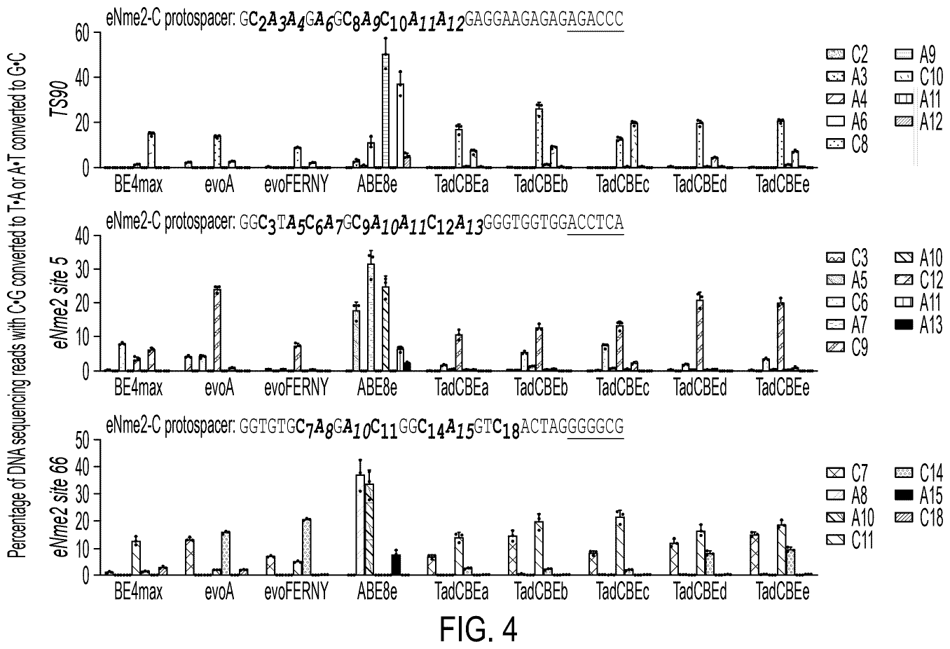

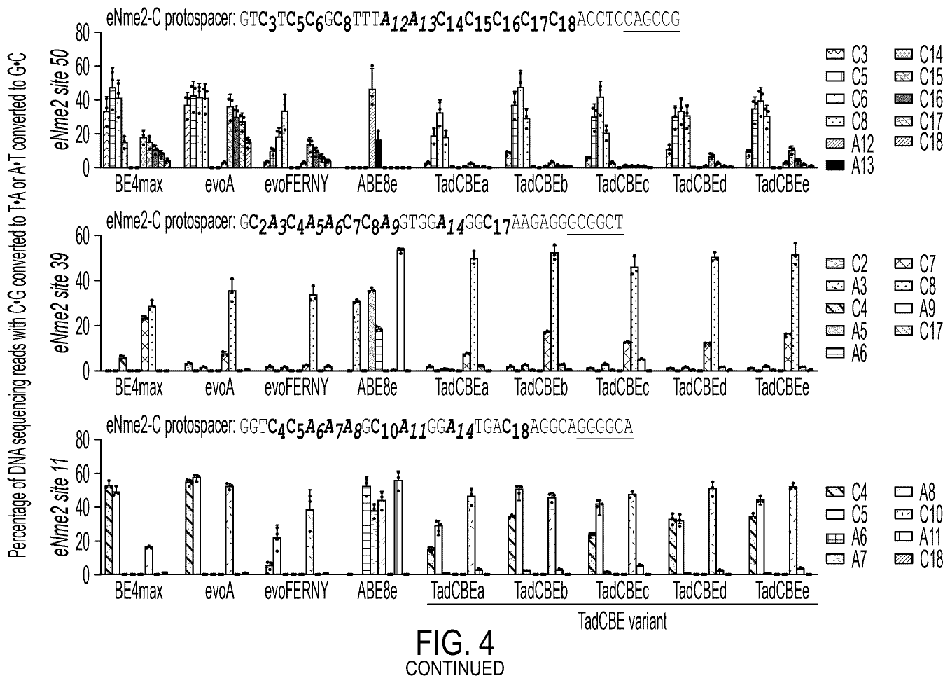

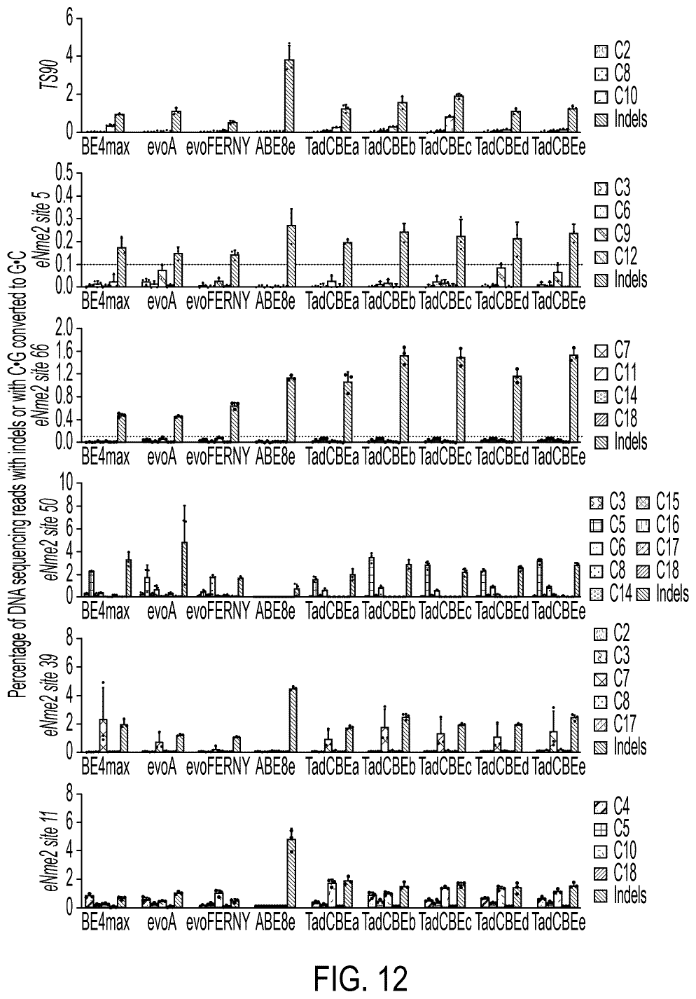

To test if TadCBEs were compatible with Cas9 homologs beyond Streptococcus pyogenes Cas9, TadCBE variants with PACE-evolved variants of Nme2Cas9 from Neisseria meningitidis that broaden the scope of accessible PAMs beyond the canonical NGG PAM of SpCas9⁵⁰ were constructed. Nme2Cas9 variants were evolved that access a wide range of single-pyrimidine PAM sites as nucleases or as base editors⁵¹ (see Huang, T. P. et al. Nature Biotechnology (2022), incorporated herein by reference). Fusions of TadA-CDs with eNme2-C nickase (PAM=N₄CN) and two UGI domains were generated, the resulting eNme2-C-TadCBEs were co-transfected with a guide RNA plasmid, and the base editing at six genomic loci in HEK293T cells was examined. Across all tested sites, the peak editing efficiency of TadCBEs was comparable to that of APOBEC1, evoFERNY, and evoAPOBEC1 (FIGS. 4, 12). Although C·G-to-T·A editing exceeded 50% at some sites, residual A·T-to-G·C editing never exceeded 5.3% at any of the six eNme2 target sites tested. TadCBEs thus exhibited robust activity and selectivity with eNme2 Cas9. These observations suggested potential compatibility with other Cas proteins that together with SpCas9 and eNme2-C Cas9 may offer access to a variety of PAM sequences for versatile targeting of TadCBEs.

### Example 4. On-Target and Off-Target Editing by TadCBEs and V106W Variants

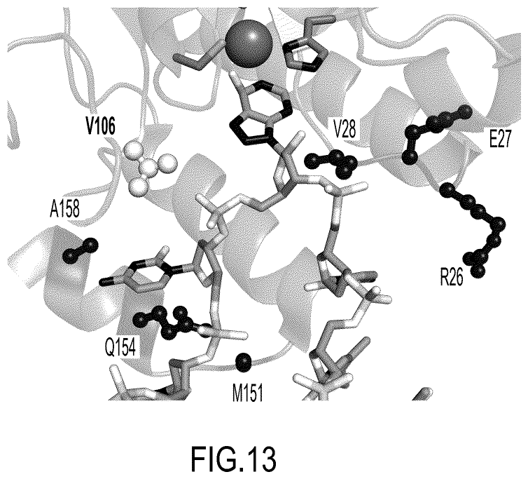

The TadA origin of TadCBEs offers several advantages for minimizing off-target editing, including the potential to include mutations that were found to reduce off-target DNA or RNA editing in previous TadA engineering efforts^{34,58,59}. For ABEs, the addition of V106W to TadA-7.10, TadA-8e, or TadA-8.17-m reduced Cas-independent off-target editing of DNA and RNA in all three cases while maintaining high levels of on-target activity^{8,9,34}. Whether the V106W mutation could reduce off-target DNA or RNA editing when introduced into TadCBEs while maintaining on-target activity and selectivity was tested. Because several evolved mutations in TadA-CDs were proximal to V106, it was not clear if the addition of V106W would disrupt desired TadA-CD properties (FIG. 13).

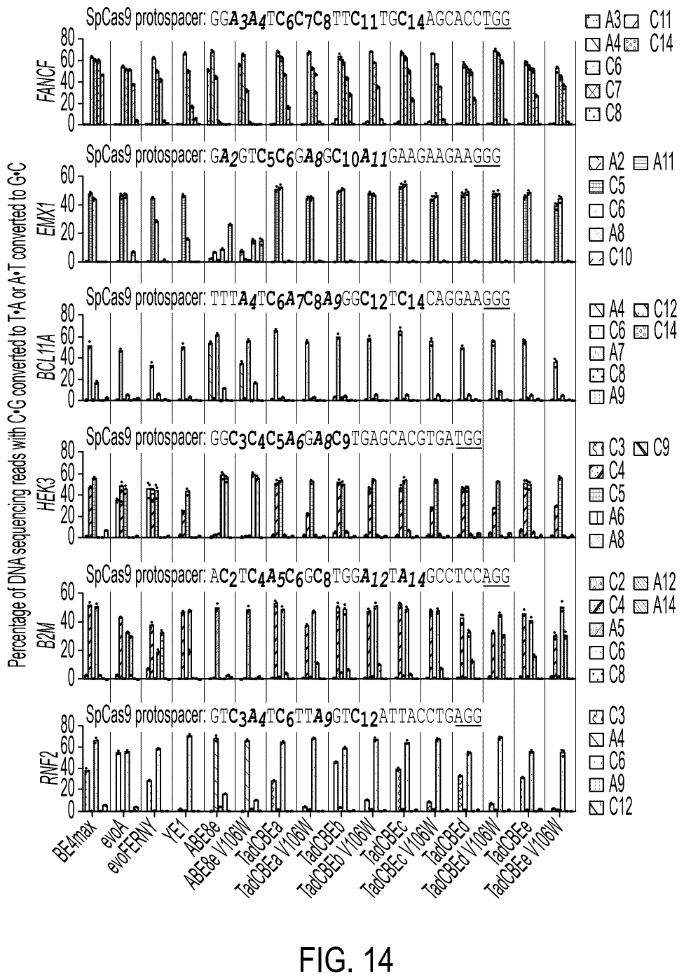

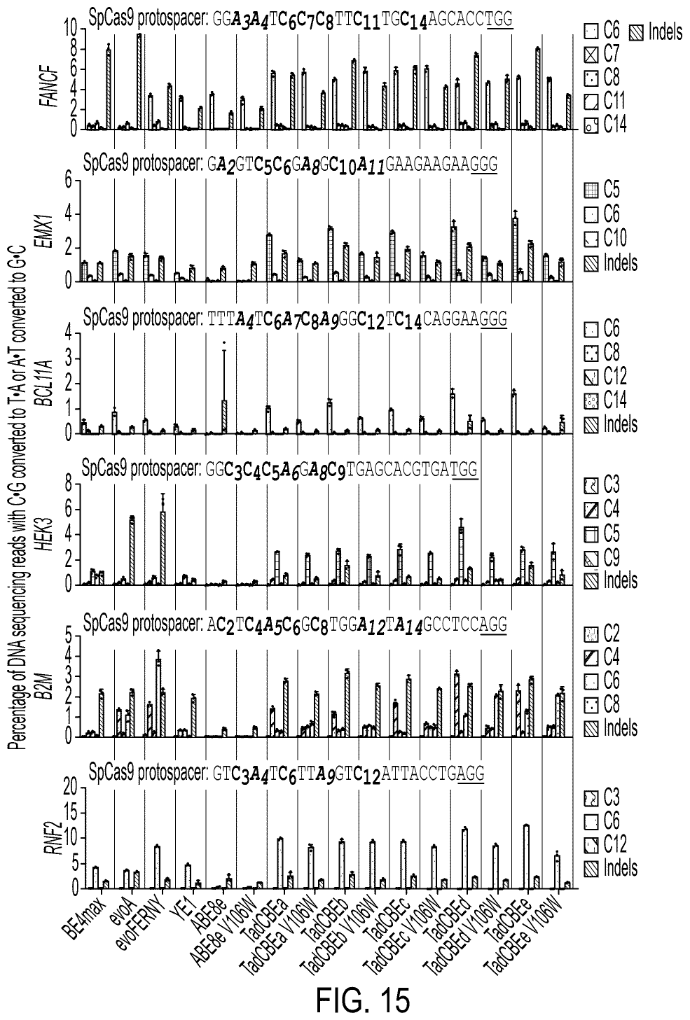

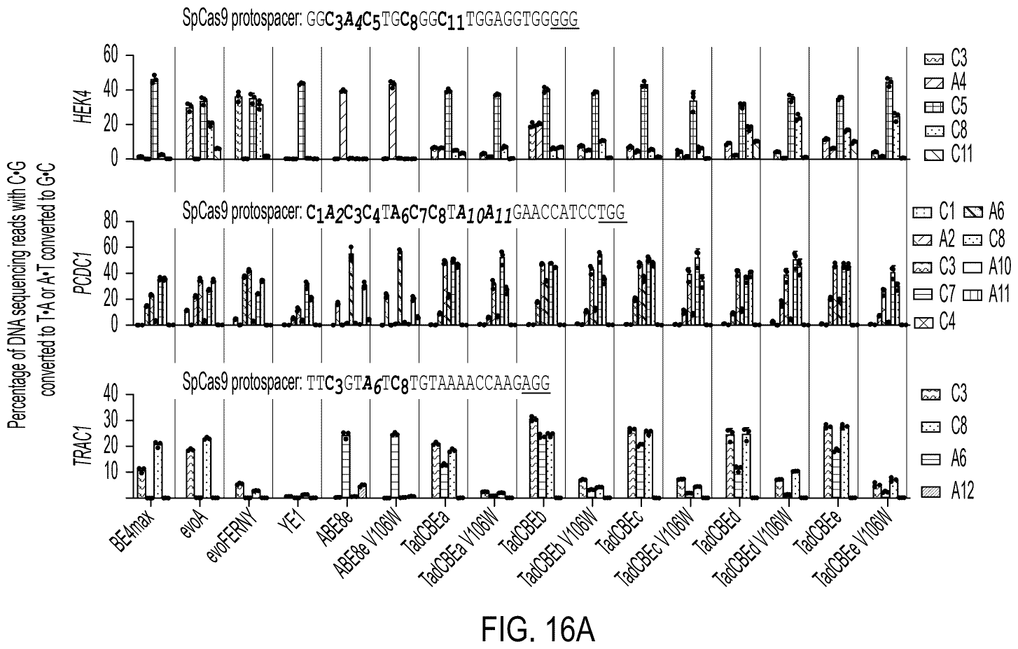

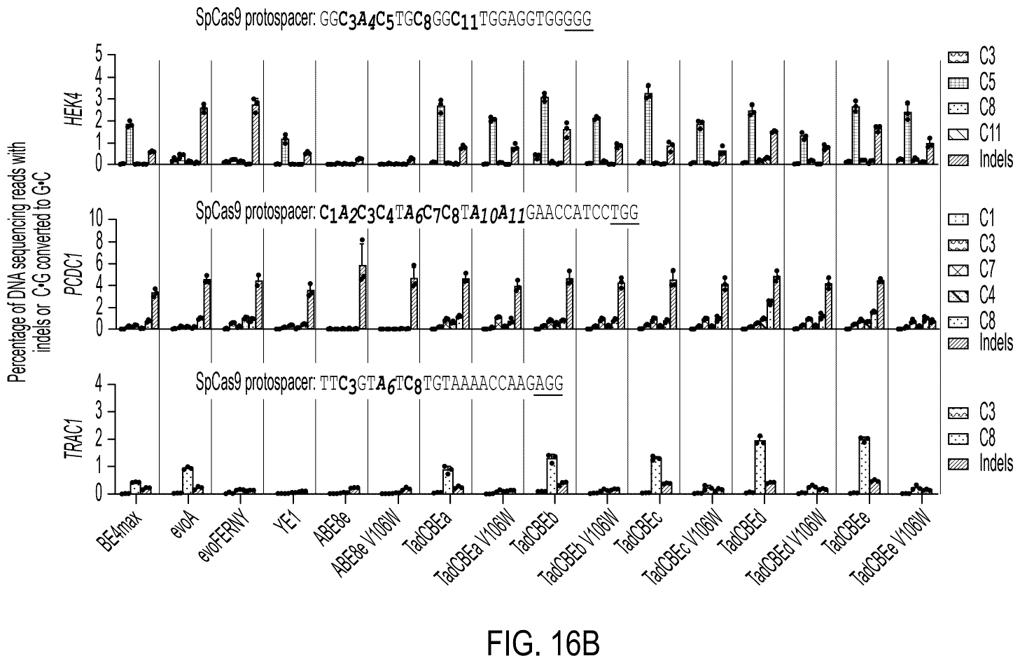

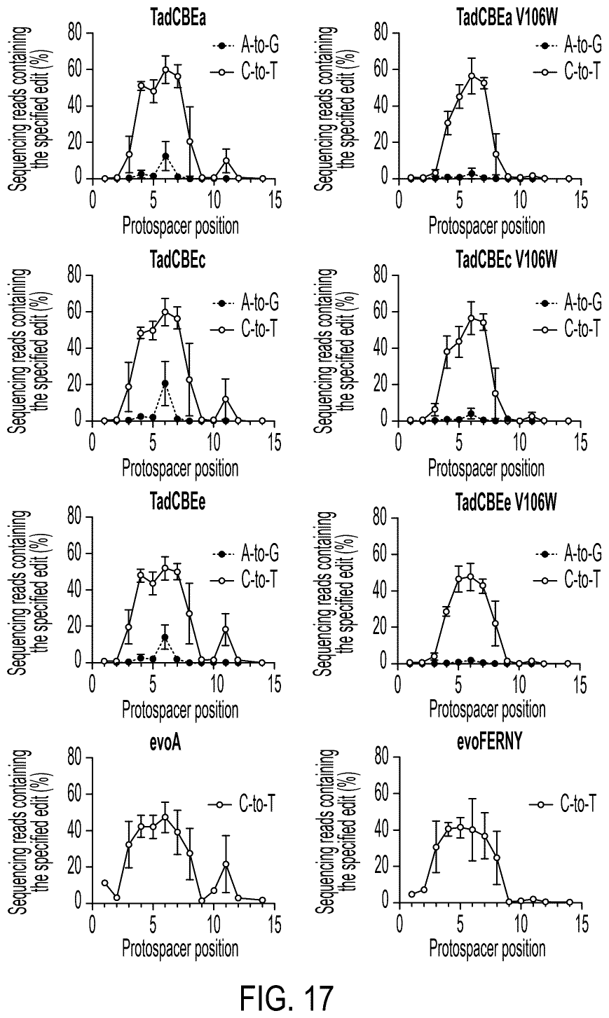

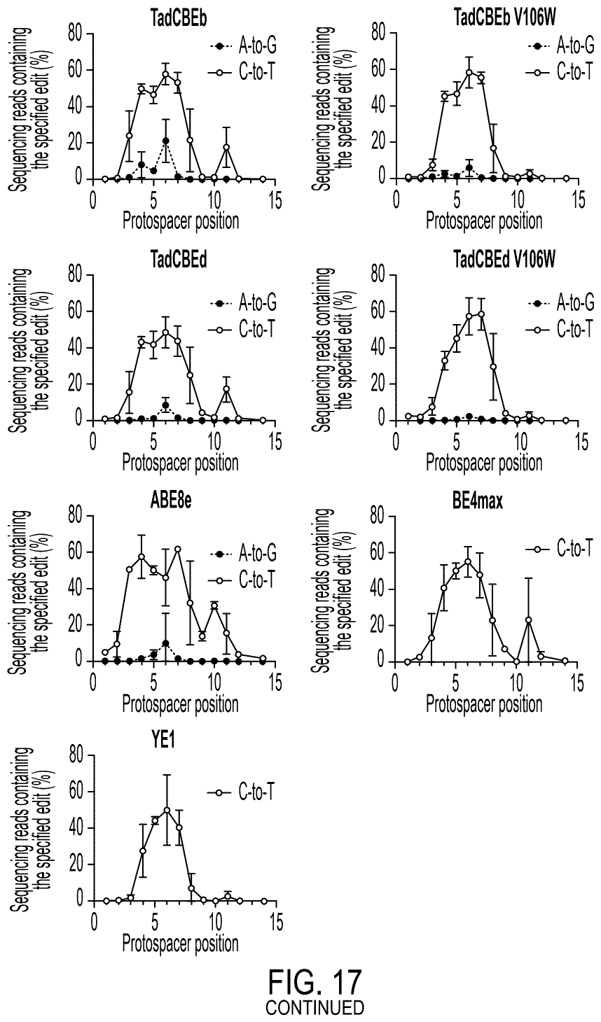

First, the on-target activity of TadCBEs containing V106W was evaluated. V106W variants of TadCBEa-e were constructed and their editing efficiency at nine target sites in HEK293T cells was evaluated. TadCBE variants a through e, tolerated the addition of V106W and maintained high on-target cytidine deamination activity, averaging 56% peak C·G-to-T·A target editing efficiency across the nine tested target sites for TadCBEa-d V106W, nearly matching 57% average peak editing efficiency for TadCBEa-d (FIG. 5A, FIGS. 14-17). The TadCBEa-e V106W variants exhibited a slightly narrower editing window than TadCBEa-d, while maintaining high peak editing efficiency (FIG. 17). Encouragingly, cytosine versus adenine base editing selectivity was improved 3.1-fold on average for TadCBE V106W variants compared to the corresponding TadCBE variants across these nine sites (FIG. 17). TadCBE-V106W variants thus retained efficient cytosine base editing with improved selectivity for cytidine over adenosine deamination and refined editing windows.

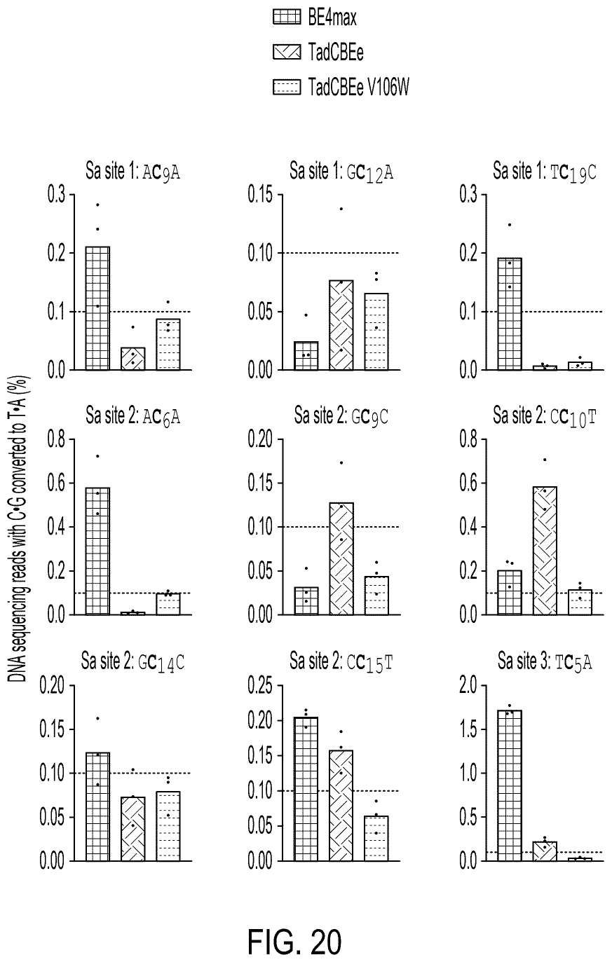

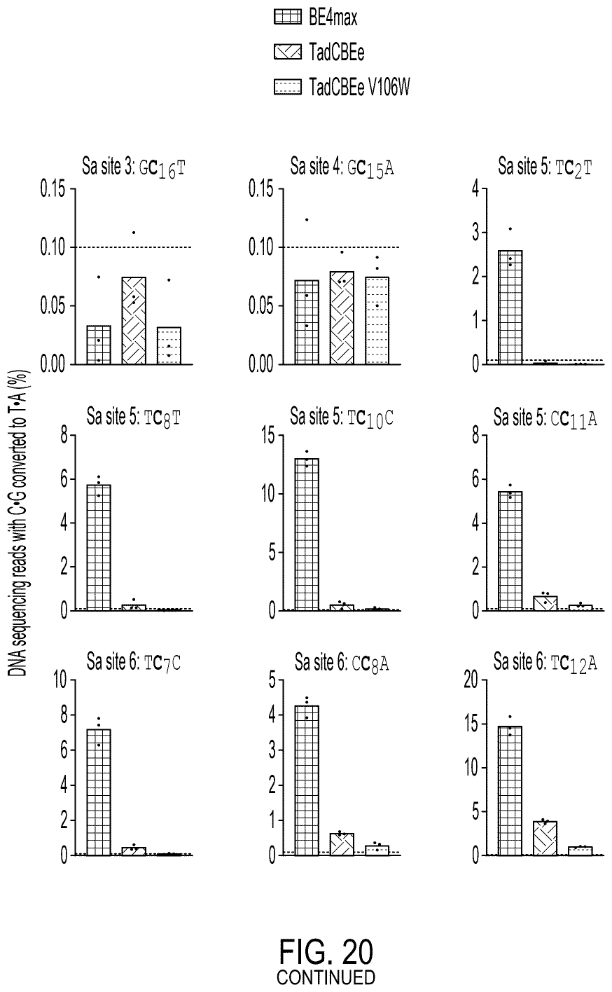

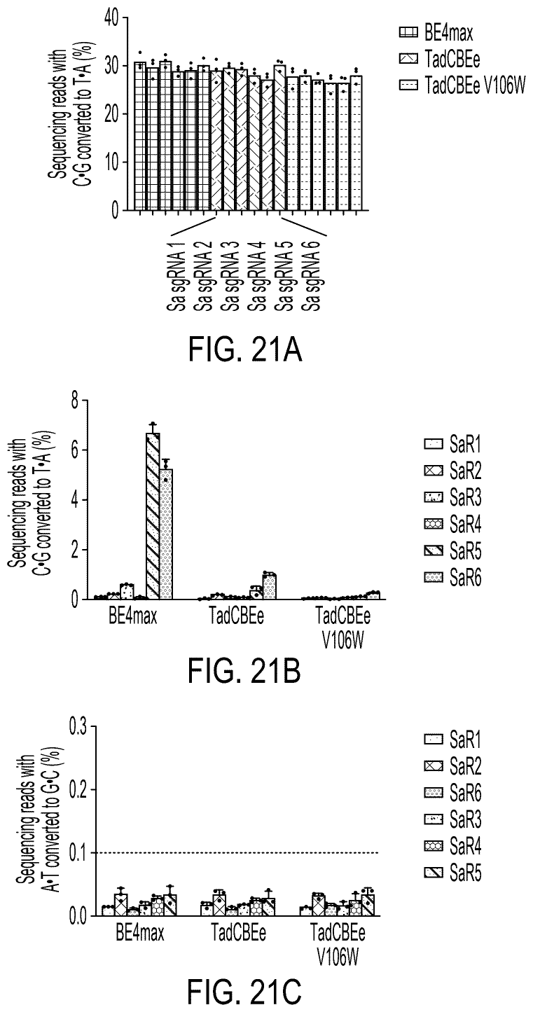

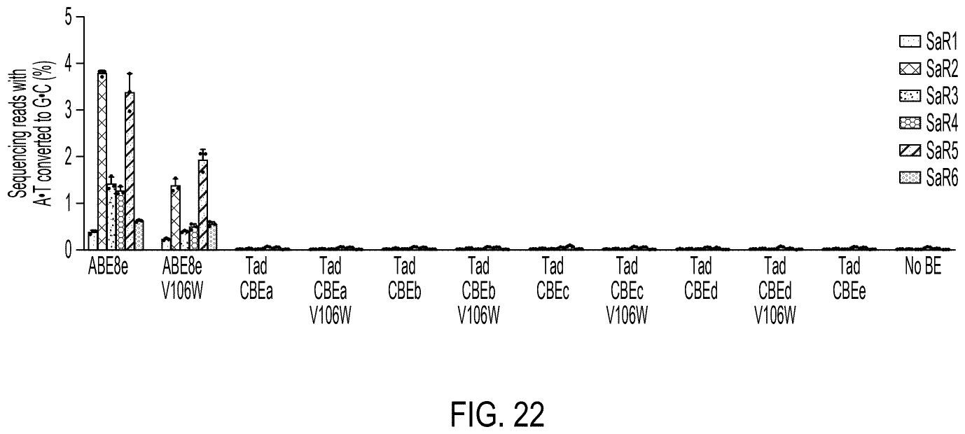

Next, Cas-independent DNA editing by TadCBEs and TadCBE-V106W variants was evaluated using the previously established orthogonal R-loop assay^{15,19} (FIG. 5B). This assay measured the propensity of a base editor to modify ssDNA in an off-target R-loop generated by an orthogonal, catalytically inactive S. aureus Cas9 (SaCas9). By sequencing genomic DNA across six unrelated off-target SaCas9 R-loops, it was determined that TadCBEs on average have 3.7-fold lower Cas-independent off-target C·G-to-T·A editing (0.84%-1.2%) compared to BE4max (3.6%) and evoA (3.8%) (FIG. 5C, FIGS. 18-21C). The average off-target activity of evoFERNY (0.58%) and YE1 (0.53%) were also low. The addition of V106W further reduced Cas-independent off-target editing of TadCBEs by an average factor of 1.9 (to 0.38%, 0.62% 0.48%, 1.1%, and 0.05% for V106W TadCBE variants a through e, respectively). Consistent with the selectivity of TadCBEs for cytidine deamination, appreciable off-target A·T-to-G·C editing by any TadCBEs was not detected (FIG. 22). These findings indicated that evolved TadCBEs had inherently low Cas-independent editing off-target DNA editing that could be further suppressed by adding V106W, while retaining high on-target C·G-to-T·A editing and low residual A·T-to-G·C editing.

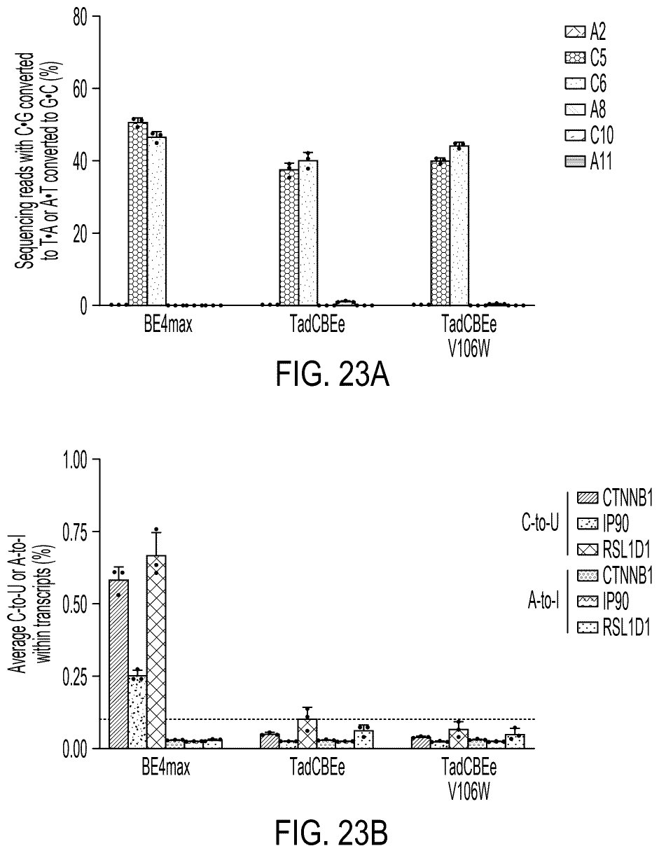

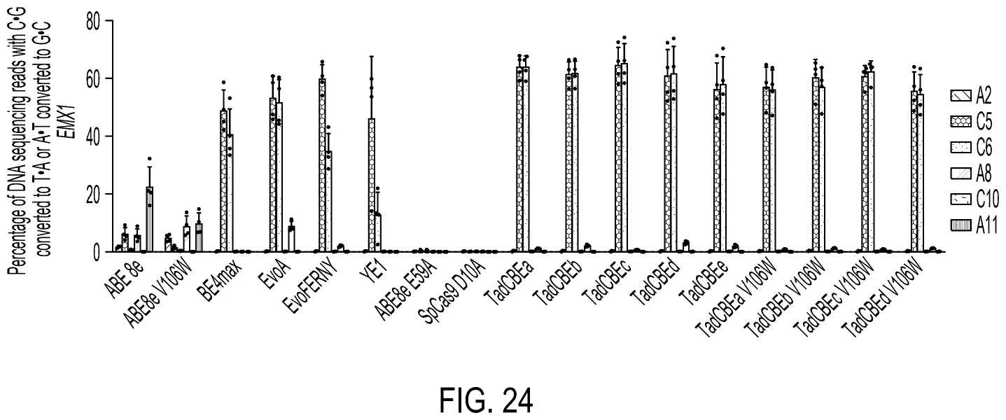

Off-target RNA editing by TadCBEs was also evaluated (FIG. 5D, FIGS. 23A-23B, and FIG. 24). Following transfection of HEK293T cells by TadCBEa-e, BE4max, evoA, evoFERNY, ABE8e, or ABE8e-V106W, RNA was extracted from cells. Following cDNA synthesis, three target transcripts (CTNNB1, IP90, and RSL1D1), which were previously used to measure off-target RNA editing due to their abundance or sequence similarity to the native TadA tRNAArg2 substrate,^{4,15,19,34} were amplified by RT-PCR and analyzed for C-to-U or A-to-I editing by high-throughput sequencing. While BE4max and evoA edited ˜0.7% of the analyzed cytosines in these transcripts, evoFERNY, YE1, and TadCBEa, TadCBEb, and TadCBEc all edited ≤0.1% of the cytosines (the limit of detection) (FIG. 5D, FIGS. 23A-23B). TadCBEd and TadCBEe edited on average 0.3% and 0.2% of cytosines across the three transcripts, respectively. The addition of V106W reduced the average off-target RNA editing down to ≤0.13% for both cases (FIG. 5D, FIGS. 23A-23B).

Taken together, these data suggested that TadCBEs offered much lower frequencies of Cas-independent off-target DNA and RNA editing compared to BE4max and evoA. Off-target editing by TadCBEs is substantially less frequent than that of any other CBE of comparable on-target activity and size. When further reduction of off-target editing was essential, the addition of V106W minimized off-target DNA and RNA editing, focused the editing window to ˜4-5 bp, and minimized residual adenosine deamination, with only a small reduction in maximal on-target activity.

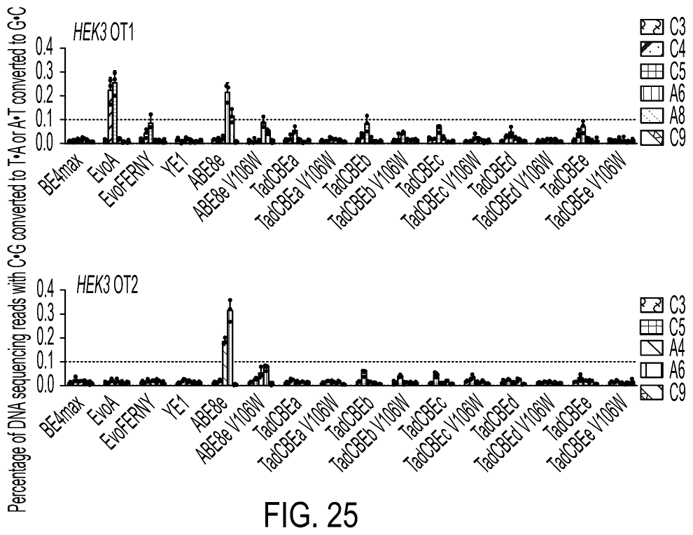

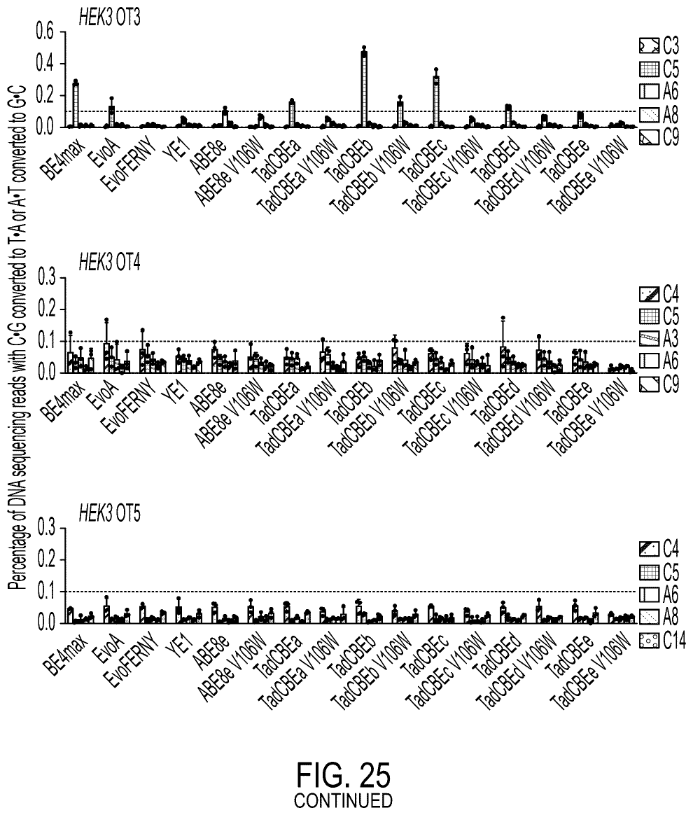

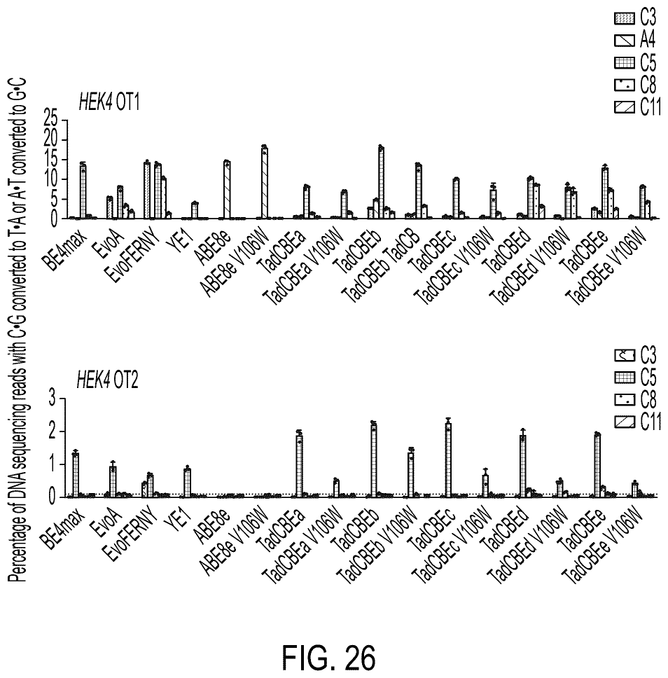

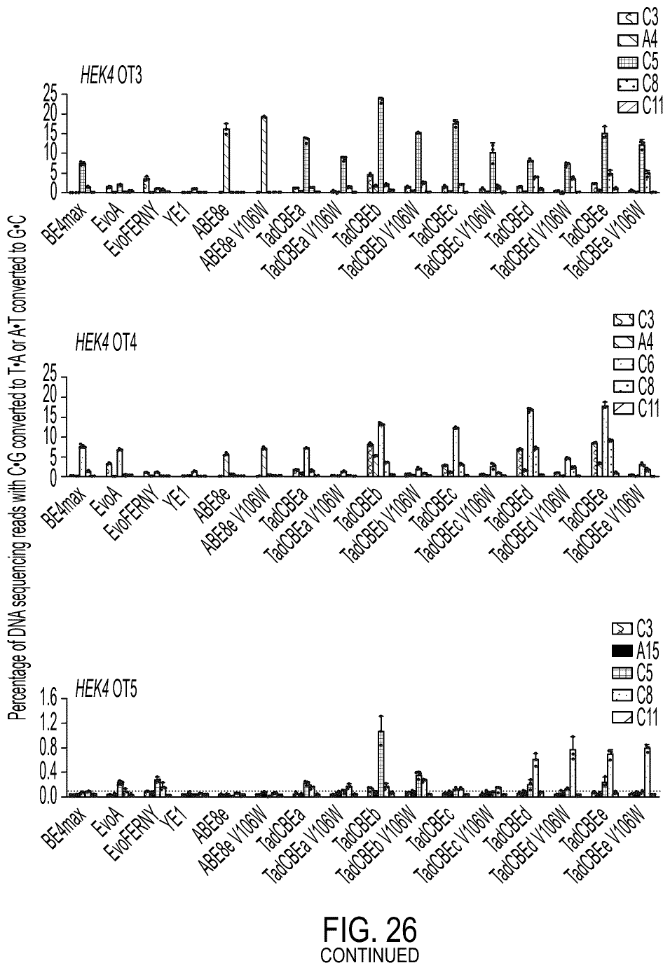

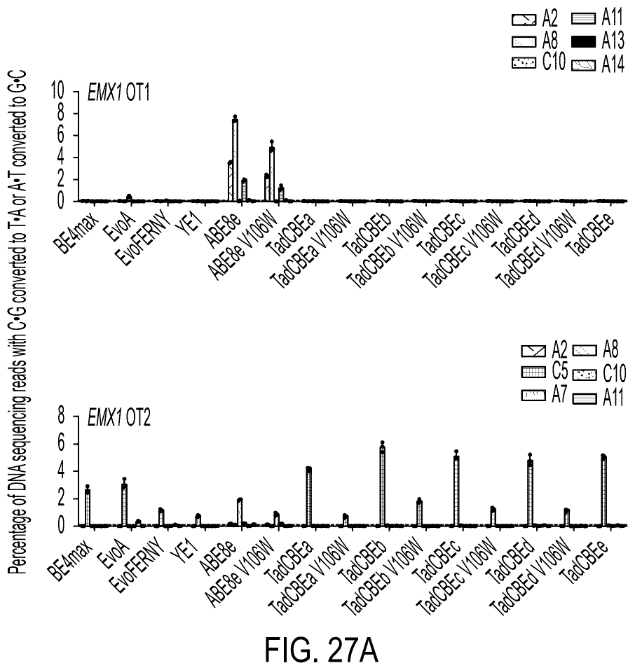

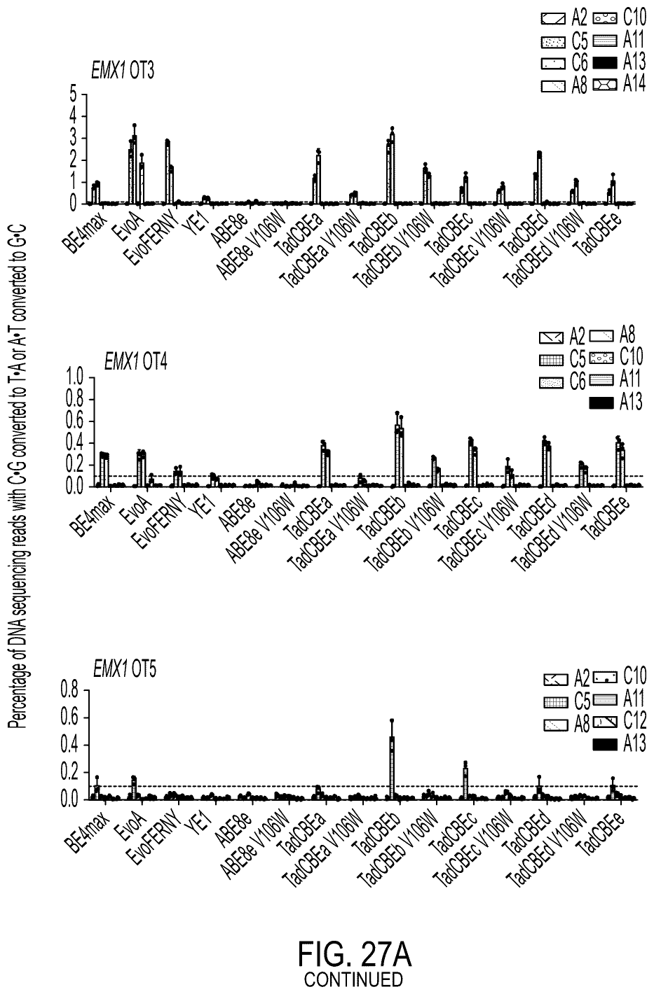

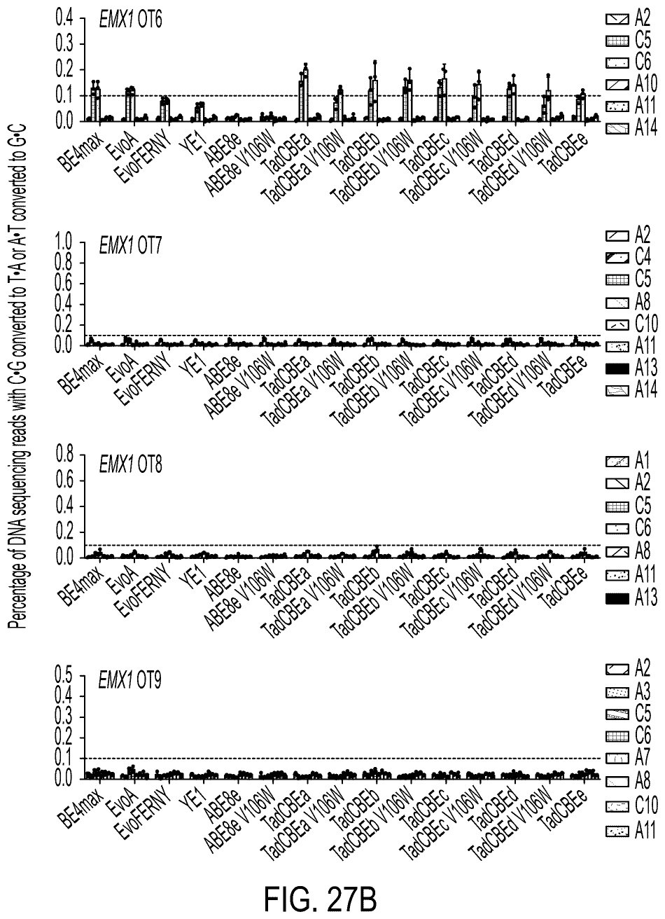

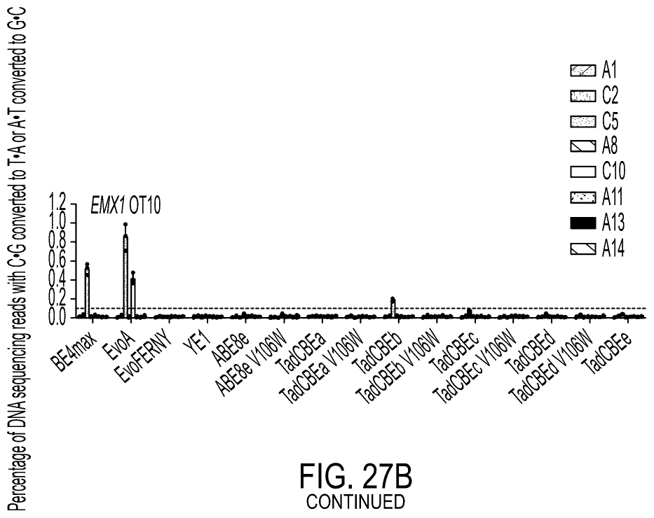

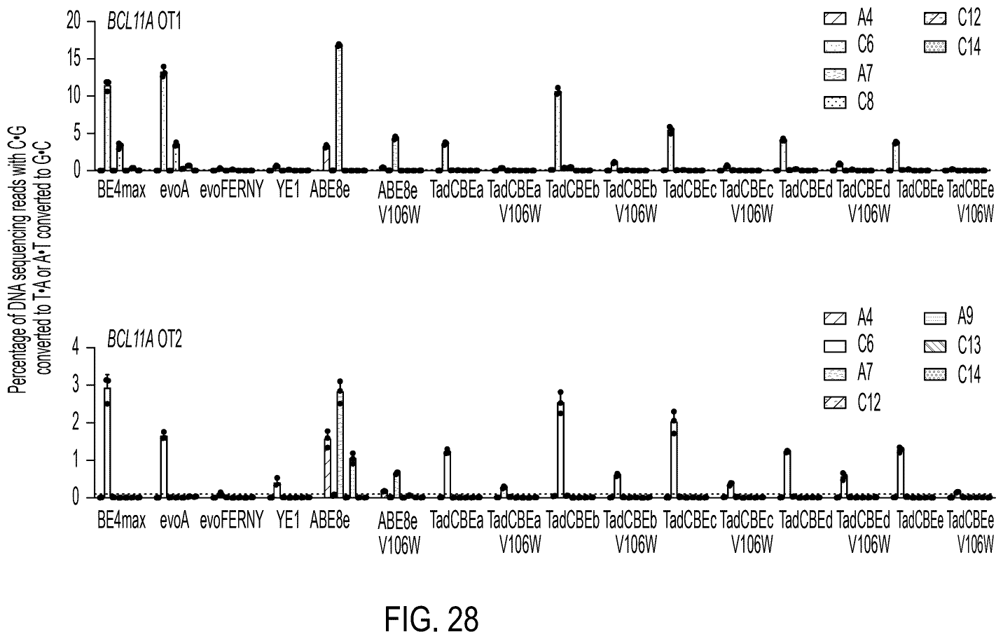

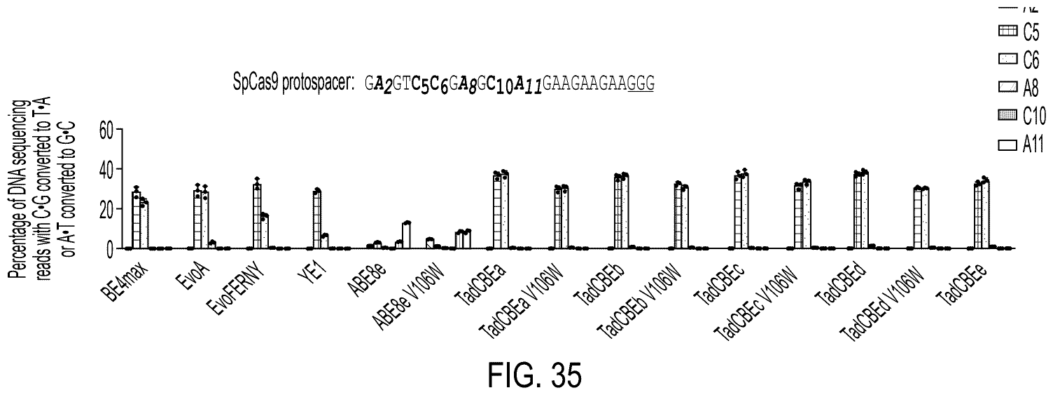

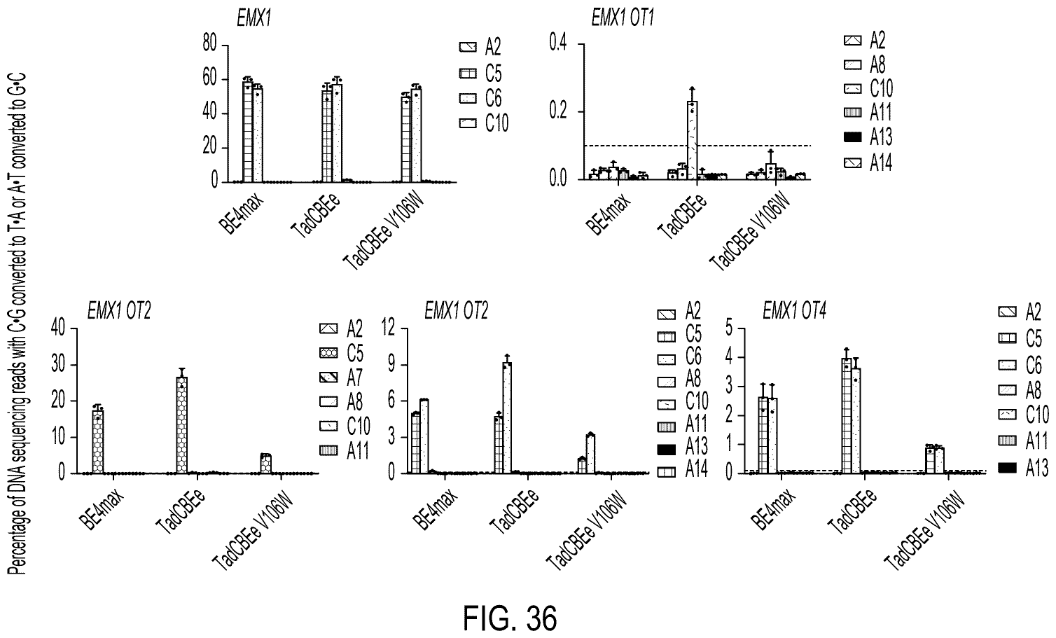

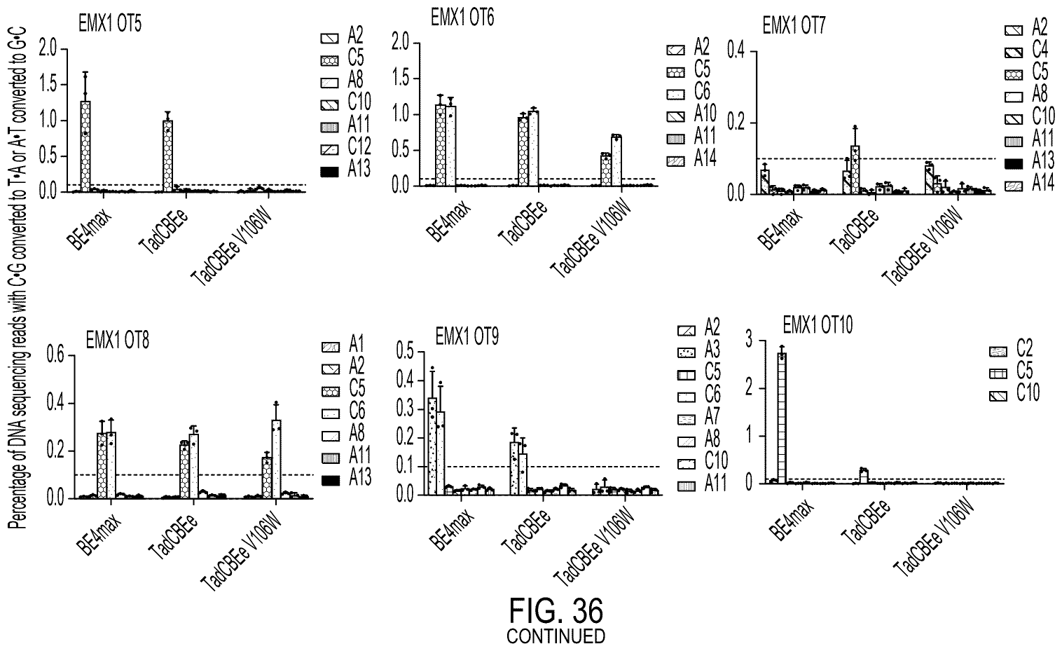

Finally, Cas-dependent off-target editing occurred when base editors engaged a non-target site that resembled the target site through imperfect Cas9 binding⁶⁰. Cas-dependent off-target activity was also analyzed in HEK293T cells at 22 known off-target sites for SpCas9 base editors and sgRNAs targeting HEK293T site 3 (hereafter referred to as HEK3), HEK293T site 4 (hereafter referred to as HEK4), EMX1, and BCL11A (FIGS. 25-28, FIGS. 35-36). Across multiple validated off-target sites, Cas-dependent off-target editing by TadCBEs was observed to be generally similar to the low level of editing observed for BE4max and EvoA variants (FIGS. 25-28, FIGS. 35-36). The Cas-dependent off-target activity of YE1 and evoFERNY was still lower, consistent with the lower on-target activity of these variants (FIGS. 25-28, FIGS. 35-36).

Collectively, these findings suggested that TadCBEs offered improved Cas-independent off-target DNA and RNA editing compared to canonical CBEs, and low levels of Cas-dependent off-target DNA editing consistent with those observed for currently used CBEs of similar on-target editing efficiencies. The use of high-fidelity Cas proteins that engage fewer off-target loci is known to reduce Cas-dependent off-target DNA base editing⁶¹, and their use in TadCBEs may offer the same benefits.

### Example 5. Multiplexed Base Editing at Therapeutically Relevant Loci in Primary Human T Cells, and Base Editing at a Therapeutically Relevant Site in Human Hematopoietic Stem Cells

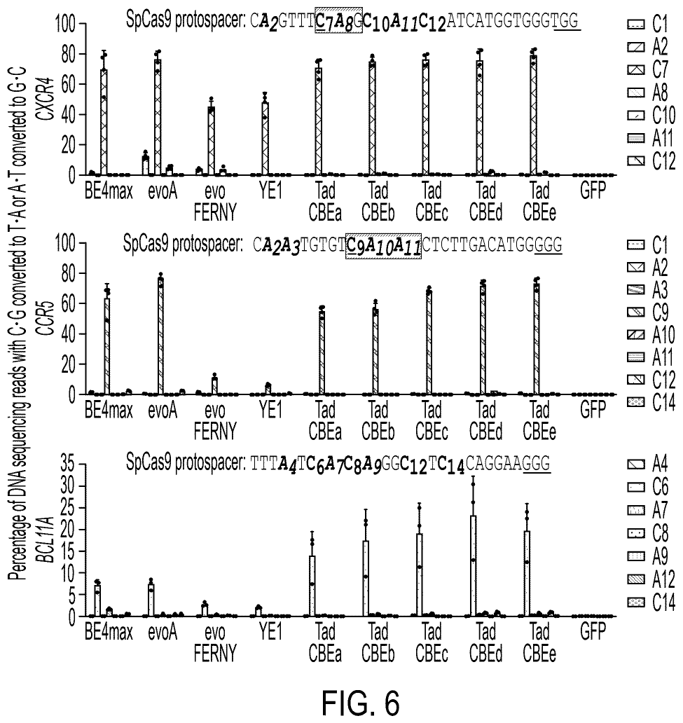

Whether TadCBEs could perform multiplexed editing of target loci in T cells in support of therapeutic applications was evaluated. Multiplexed base editing in T cells can be used to modify or disrupt multiple genes without the risk of chromosomal abnormalities and cell-state perturbations that arise from multiple double-stranded breaks⁵⁸⁻⁶². To determine whether TadCBEs could perform multiplexed editing in primary human T cells, the CXCR4 and CCR5 loci were targeted for simultaneous base editing to install premature stop codons in both HIV co-receptors (FIG. 6)⁶³. In vitro transcription of TadCBE variants a, b, c, d, and e was performed. Then, the TadCBE mRNA was electroporated along with guide RNAs targeting CXCR4 and CCR5 (FIG. 6)⁶³ into primary human T cells and editing efficiencies were analyzed at both target sites.

TadCBEs performed efficient (averaging 70%) and selective editing of the target cytosines (C7 in CXCR4, C9 in CCR5), resulting in premature stop codon installation in each gene (FIG. 6). Editing efficiencies of TadCBEs were similar to those of BE4max (67%) and evoA (76%) (FIG. 6). Observed indel frequencies of all the tested base editors were comparably low (typically ≤0.68%, FIGS. 29A-29B). Consistent with data in HEK293T cells (FIG. 17), TadA-CDs exhibited a more precise editing window with fewer bystander edits at CXCR4 and CCR5 in primary human T cells. Since TadCBEs maintained high editing efficiencies and product purities but offered substantially lower Cas-independent off-target DNA and RNA editing than APOBEC and evoA (FIGS. 5C-5D and FIGS. 18-22), TadCBEs provided a promising alternative for multiplexed cytosine base editing of T cells.

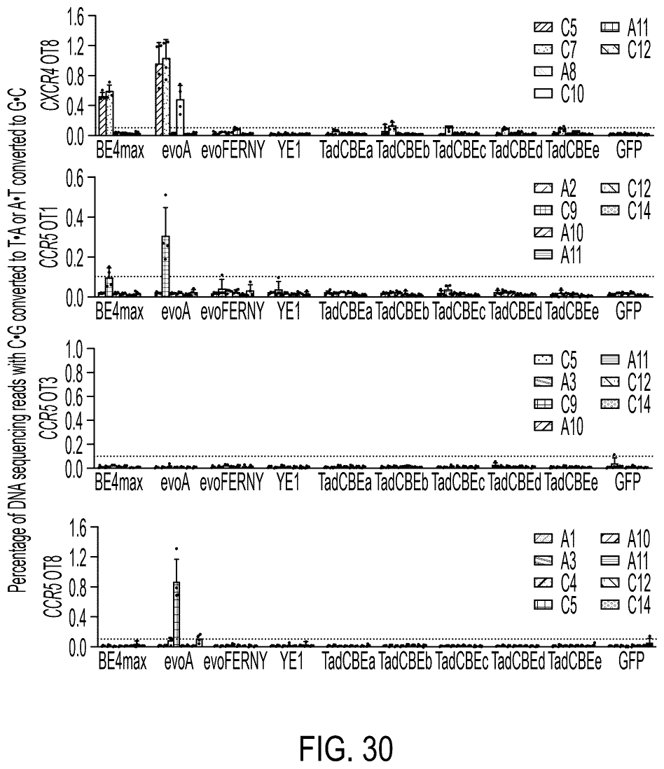

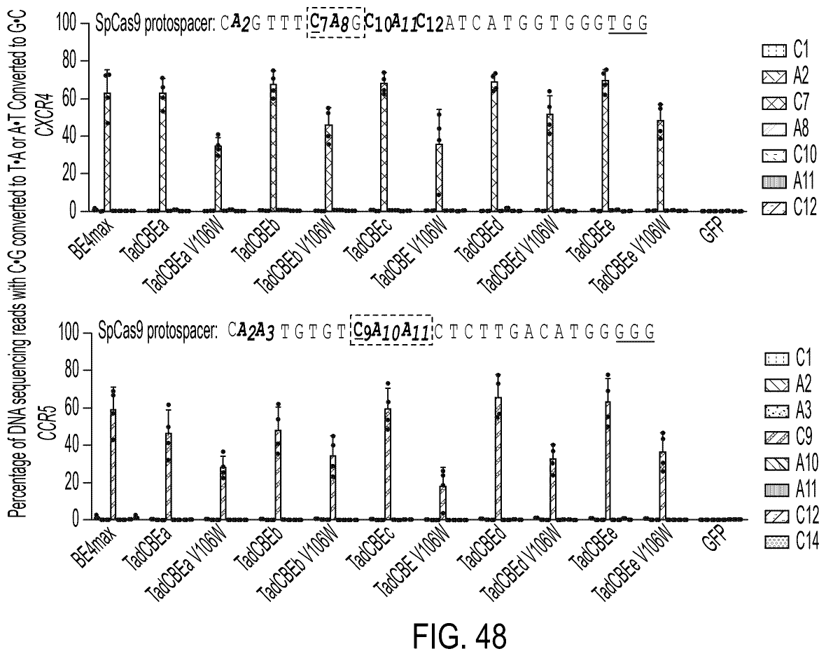

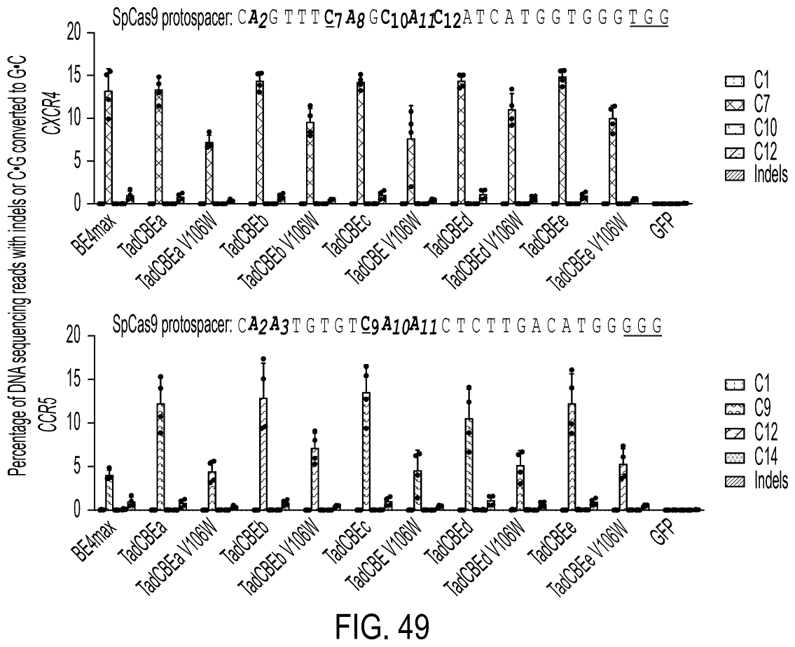

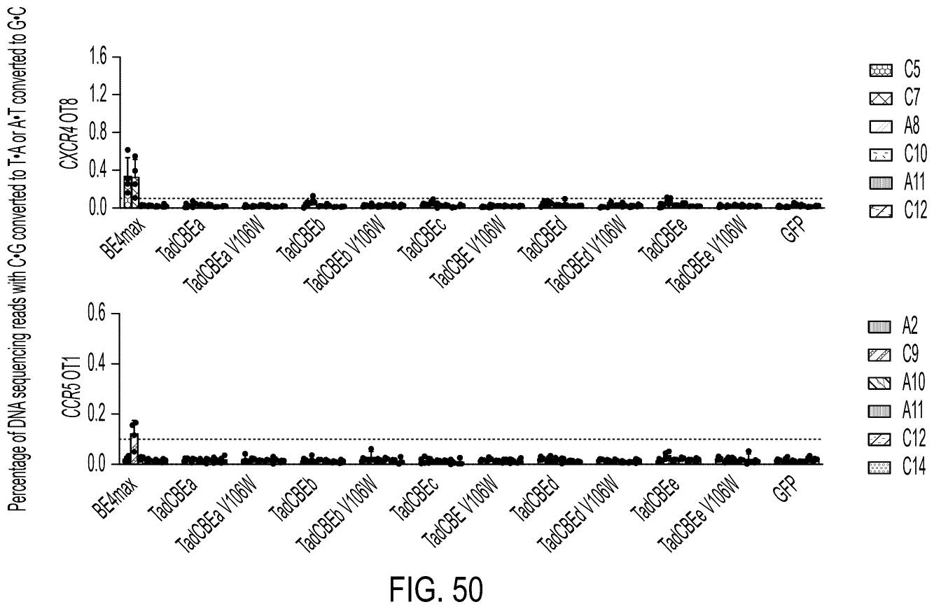

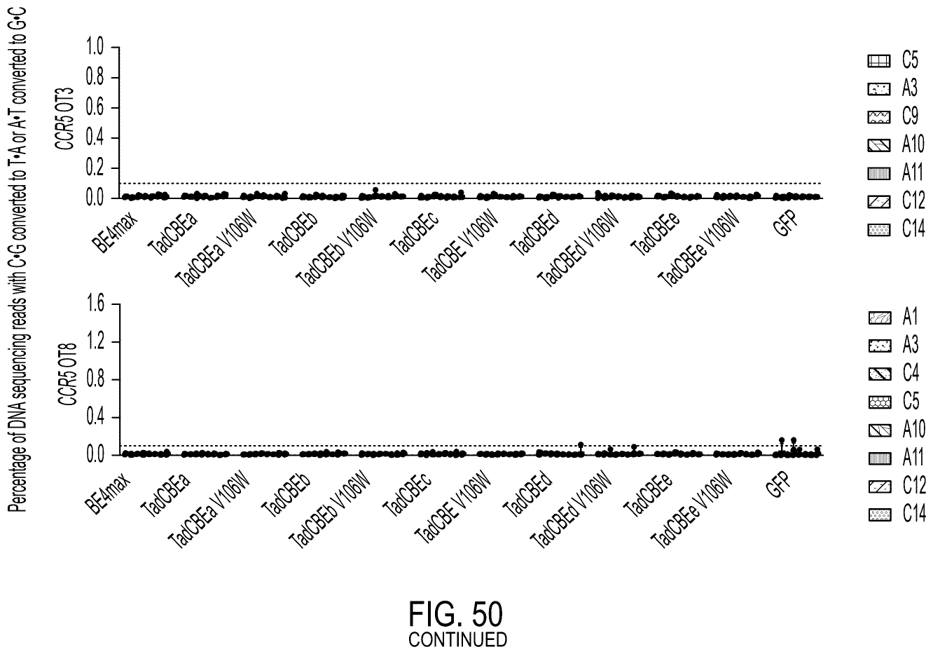

T-cell editing by TadCBEs was also compared to that of evoFERNY and YE1, which offered similarly low off-target editing as TadCBEs (FIGS. 5C-5D, FIG. 6, and FIGS. 18-22). TadCBEs supported substantially higher editing efficiencies in T cells than evoFERNY and YE1. At CXCR4, target C·G-to-T·A editing efficiency by TadCBEs averaged 1.5- to 1.7-fold that of evoFERNY and YE1, while at CCR5, average TadCBE editing efficiencies were 4.9- to 11-fold higher on average. Three known Cas-dependent off-target sites and one known off-target for CXCR4 were analyzed for the CCR5 guide RNA. Cas-dependent off-target editing was lower for TadCBEa-e, evoFERNY, and YE1 (≤0.12%) than for BE4max (0.1-0.58%) and evoA (0.1-1.0%) (FIG. 30). Next, V106W variants of TadCBEa-d were tested in T cells. Relative to their TadCBE counterparts, the V106W variants displayed 1.3- to 1.9-fold lower average activity at C7 of CXCR4 and 1.4- to 3.3-fold lower average activity at C9 of CCR5, with a proportional drop in C·G-to-G·C editing (FIGS. 48-50). These data are consistent with the narrower editing window of V106W variants and suggests that the more transient mRNA delivery of TadCBEs may reveal a greater range of editing activity compared to plasmid transfections of HEK293T cells. Overall, these findings demonstrated that TadCBEs offered a favorable combination of on-target and off-target editing features compared to currently used CBEs when base editing primary human T-cells at target sites of therapeutic relevance.

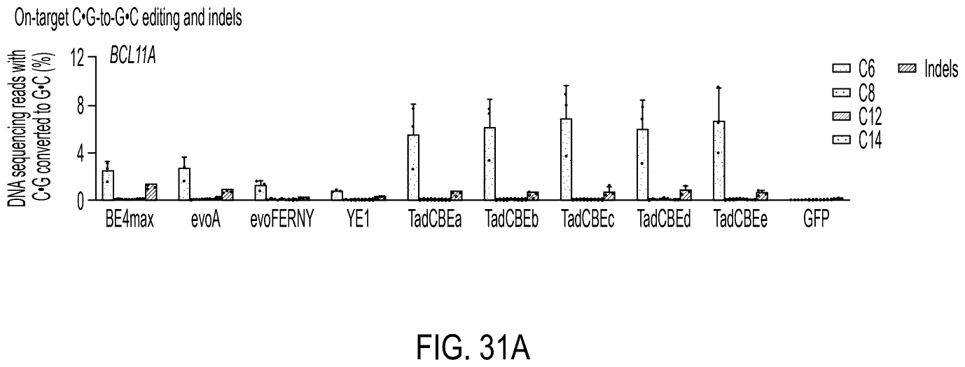

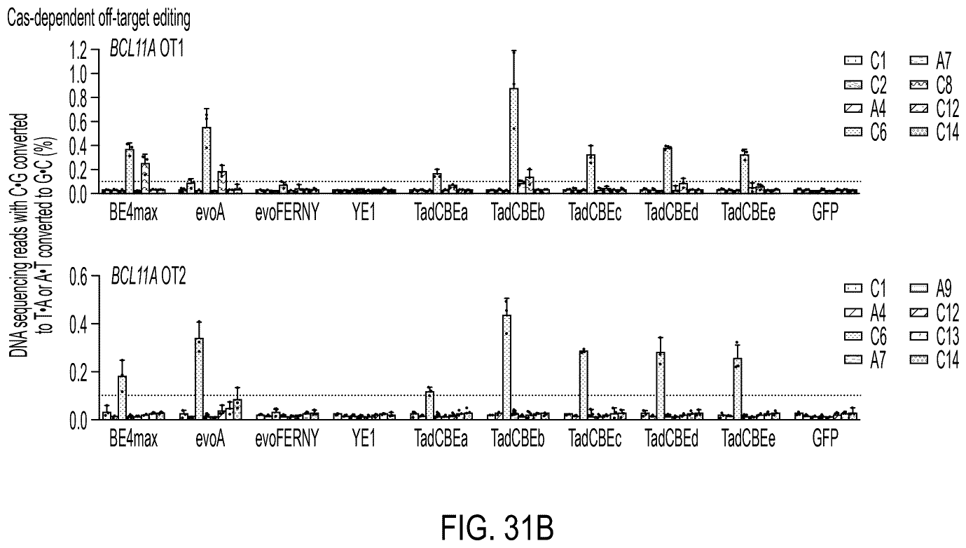

Finally, the editing efficiency of TadCBEs was evaluated in human hematopoietic stem and progenitor cells (HSPCs). TadCBEa-e mRNA was electroporated along with a synthetic guide targeting the enhancer of BCL11A into primary human CD34-positive cells. Mutations at the enhancer can decrease the expression of BCL11A, leading to induction of fetal hemoglobin expression as a potential treatment for sickle cell disease^{73,74}. For comparison, mRNA encoding BE4max, evoAPOBEC1 (evoA), evoFERNY, YE1, or GFP (as a negative control) was electroporated in parallel. evoFERNY and YE1 yielded only 2.0% and 2.7% average editing, respectively, while BE4max and evoA averaged 7.0% and 7.4% editing efficiencies, respectively (FIG. 6). All five of the tested TadCBEs supported 2- to 3-fold higher editing efficiencies than BE4max or evoA, averaging 14%-23% (FIG. 6). All of the tested CBEs yielded low levels of indels (≤1.1%, FIG. 31A) and Cas-dependent off-target editing ((≤0.87%, FIG. 31B). These results demonstrated that the editing efficiencies of TadCBEs can exceed that of the most commonly used CBEs for some therapeutically relevant sites and cell types.

### Discussion

TadA has been evolved and engineered in the laboratory from a tRNA-editing enzyme found in E. coli into widely used adenine base editors, including several that are already in the clinic² or headed to clinical trials¹. Evolved TadA variants offer many characteristics that are beneficial for precision gene editing applications, including some features not previously present in cytosine base editors. The evolution of new TadA variants that catalyze efficient and selective cytidine deamination in this study enabled the development of TadCBEs, a new class of CBEs that offer high on-target editing, low off-target Cas-independent and Cas-dependent DNA editing, low off-target RNA editing, and size small enough to fit into a single AAV^{27,28}. In HEK293T cells, TadCBEs perform highly efficient C·G-to-T·A editing across a range of sites with both SpCas9, Nme2Cas9 and SaCas9. These results represent the first directed evolution of a deaminase to selectively deaminate a different base, rather than simply relaxing target base specificity—an outcome of the simultaneous positive and negative selection system that evolved selective TadCBE deaminases.

A side-by-side comparison with commonly used CBEs revealed that TadCBEs offer unique properties that make them well-suited for applications where canonical BE4max, evoA, evoFERNY, and YE1 may face limitations. The narrow editing window of TadCBEs is beneficial when precision editing is required. Despite having comparable on-target editing efficiencies as BE4max and evoA, TadCBEs exhibit substantially lower Cas-independent off-target DNA and RNA editing. evoFERNY and YE1 also exhibit low Cas-independent editing, but display different editing profiles and achieve substantially lower editing efficiency at some target loci, including CXCR4 and CCR5 in T cells, and BCL11A in HSPCs. The evolution of TadA-CDs from TadA-8e therefore extends the utility of TadA for gene editing, demonstrates a new strategy for generating base editors, and provides a novel family of CBEs with favorable editing properties.

TadCBEd offers the highest on-target editing and and selectivity of the TadCBE variants for general cytosine base editing applications. When off-target DNA or RNA editing or residual A·T-to-G·C editing must be kept to a minimum, TadCBEd-V106W is recommended.

### Methods

### General Methods and Molecular Cloning

Gibson assembly (New England BioLabs) or USER cloning (New England BioLabs) was used to carry out all plasmid construction. Nuclease-free water (Qiagen) was used for PCR reactions and cloning. For all other experiments, water was purified using a MilliQ purification system (Millipore). PCR was performed using Phusion HiFi polymerase or Phusion U Green Hot Start II DNA polymerase (ThermoFisher Scientific). Following Gibson or USER cloning, cloning products were transformed into Mach 1 chemically competent E. coli (ThermoFisher Scientific). Selection antibiotics were used at the following final concentrations: carbenicillin: 100 μg/ml; spectinomycin: 50 μg/ml; kanamycin: 50 μg/ml; chloramphenicol: 25 μg/ml; tetracycline: 10 μg/ml. Plasmid DNA was amplified using the Illustra Templiphi 100 Amplification Kit (GE Healthcare Life Sciences) prior to Sanger sequencing (Quintara Boston). Sequence-confirmed plasmids for bacterial transformation were purified using the Miniprep Kit (Qiagen). Plasmids for mammalian transfection were purified using the Midiprep Kit (Qiagen) according to the manufacturer's instructions. Plasmids were quantified by nanodrop. A full list of bacterial plasmids used in this work is given in Table 1.

### Bacteriophage Cloning

For USER assembly of phage, 0.2 pmol of each PCR fragment was added to a final volume of 20 μL. Following USER assembly, the 20-μL USER reaction was transformed into 100 μL of chemically competent S2060 E. coli host cells containing pJC175e⁴⁶. For Gibson assembly of phage, 0.2 pmol of each PCR fragment was added to make up a final volume of 20 μL. Following Gibson assembly, the 20 μL Gibson reaction was transformed into 100 μL of chemically competent S2060 E. coli host cells containing pJC175e⁴⁶. Cells transformed with pJC175e enable activity-independent phage propagation and were grown for 5 hours at 37° C. with shaking in antibiotic-free 2×YT media. Bacteria were then centrifuged for 1 minute at 10,000 g and plaqued as described below to isolate clonal phage populations. Individual plaques were grown in DRM media (prepared from US Biological CS050H-001/CS050H-003) for 6-8 hours. Bacteria were centrifuged for 10 minutes at 6,000 g to remove E. coli from the supernatant. The supernatant containing the phage was filtered through 0.22 μm PVDF Ultrafree centrifugal filter (Millipore) to remove residual bacteria. For sequencing, the gene of interest within the phage was amplified with primers AB1793 (5′-TAATGGAAACTTCCTCATGAAAAAGTCTTTAG) (SEQ ID NO: 270) and AB1396 (5′-ACAGAGAGAATAACATAAAAACAGGGAAGC) (SEQ ID NO: 271) and the PCR product was sequenced by Sanger sequencing (Quintara). The primers anneal to the phage backbone, flanking the evolving gene of interest. Sequence-confirmed phage were stored at 4° C.

### Preparation and Transformation of Chemically Competent Cells

Strain S2060⁷⁵ was used in all phage propagation, PANCE, and PACE experiments. To prepare competent cells, an overnight culture was diluted 250-fold into 50 ml of 2×YT media (United States Biologicals) supplemented with tetracycline and grown at 37° C. with shaking at 230 RPM to OD₆₀₀ ˜0.4-0.6 and then incubated on ice for 20 minutes. Cells were then pelleted by centrifugation at 4,000 g for 10 minutes at 4° C. The cell pellet was resuspended by addition of 5 ml of TSS (LB media supplemented with 5% v/v DMSO, 10% w/v PEG 3350, and 20 mM MgCl2). The cell suspension was pipetted gently to mix completely, aliquoted into 100-μL volumes, flash-frozen in liquid nitrogen, and stored at −80° C.

To transform cells, 100 μL of competent cells thawed on ice was added to a pre-chilled mixture of plasmid (1-2 μL each; up to 3 plasmids per transformation) in 20 μL 5×KCM solution (500 mM KCl, 150 mM CaCl2, and 250 mM MgCl2 in H2O) and 80 μL H2O and mixed gently by pipetting. The mixture was incubated on ice for 15 minutes and heat shocked at 42° C. for 90 seconds before adding 800 μL of SOC media (New England BioLabs) to rescue. Cells were allowed to recover at 37° C. with shaking at 230 RPM for 1-1.5 hours, plated on 2×YT media+1.5% agar (United States Biologicals) containing the appropriate antibiotics, and incubated at 37° C. for 16-18 hours.

### Plaque Assays for Phage Titer Quantification and Cloning

Phage were plaqued on S2060 E. coli host cells containing the pJC175e plasmid to enable activity-independent propagation⁴⁶. To prepare E. coli host cells at the appropriate grown phage for plaquing, an overnight culture of host cells (fresh or stored at 4° C. for up to 3 days) was diluted 50-fold in DRM containing the appropriate antibiotics. Cells were grown at 37° C. to an OD₆₀₀ of 0.8-1.0 (˜2 hours), at which point they were moved to an ice bucket during preparation of the phage. Phage stocks were serially diluted with DRM by a factor of 10 (up to 10⁶-fold). To prepare plates for plaquing, molten 2×YT medium agar (1.5% agar, 55° C.) was mixed with Bluo-gal (Gold Bio, 4% w/v in DCM) to a final concentration of 0.08% Bluo-gal. The molten agar mixture was pipetted into quadrants of a quartered Petri dish (2 ml per quadrant) and left at room temperature for 5 min to solidify. To prepare top agar, a 3:2 mixture of 2×YT medium and molten 2×YT medium agar (1.5%, resulting in a 0.6% agar final concentration) was prepared and stored at 55° C. until use. To plaque, 100 μL cells were mixed with 10 μL phage in 2 ml library tubes (VWR International). 900 μL of warm top agar was added to the cell and phage mixture, pipetted to mix, and then immediately pipetted onto the solid agar medium in one quarter of the petri dish. Top agar was allowed to set undisturbed for 2 minutes at 25° C. Plates were then incubated, without inverting, at 37° C. overnight. Phage titers were determined by quantifying blue plaques. For higher-throughput plaquing, the reagents were adjusted for the wells of a 12-well plate as follows: 900 μL ml bottom agar, 450 μL top agar, 10 μL phage, 100 μL cells.

### Phage Overnight Propagation Assays

S2060 cells transformed with the AP and CP plasmids of interest were prepared as described above and inoculated into DRM. Cells were grown overnight. The next day, host cells were diluted 50-fold into fresh DRM and were grown at 37° C. to an OD₆₀₀ of 0.3-0.5. Host cells were distributed into the wells of a 96-well plate (1 ml per well, Axygen), and phage of a known titer were then added to an input concentration of 105 p.f.u/ml. The cultures were grown overnight (14-20 hours) with shaking at 230 rpm at 37° C. Plates were then centrifuged at 4,000 g for 10 minutes to remove cells, leaving phage in the supernatant. The supernatants were then titered by plaquing as described above. Fold-enrichment was calculated by dividing the output propagated phage titer by the input phage concentration.

### Phage-Assisted Non-Continuous Evolution (PANCE)

PANCE experiments were performed according to published protocols⁷⁶. S2060 host cells transformed with AP and CP were made chemically competent as described above. Chemically competent host cells were transformed with mutagenesis plasmid (MP6)⁴⁷ and plated on 2×YT agar containing 100 mM glucose along with the appropriate antibiotics. Between four and eight colonies were picked into individual wells of a 96-well plate containing 1 ml of DRM and the appropriate antibiotics. The colonies were resuspended and serially diluted 10-fold, eight times into DRM. The plate was sealed with a porous sealing film and grown at 37° C. with shaking at 230 RPM for 16-18 hours. Wells containing dilutions with OD₆₀₀ ˜0.3-0.4 were combined, treated with 20 mM arabinose to induce mutagenesis, and distributed into the desired number of 1 ml cultures in a 96-well plate. The cultures were then inoculated with selection phage at the indicated dilution Table 3 and FIG. 8). Infected cultures were grown for 12-18 hours at 37° C. and harvested the next day by centrifugation at 4,000×g for 10 minutes. 100 μL of the supernatant containing the evolved phage was transferred to a 96-well PCR plate, sealed with foil, and stored at 4° C. Isolated phage were then used to infect the next passage and the process repeated for the duration of the selection. Phage titers were determined by qPCR as described previously⁷⁶ or by the plaque assay as described above. The sequences of the promoters and ribosome binding sites used during evolution are in Table 7.

### Phage-Assisted Continuous Evolution (PACE)

PACE experiments were performed according to previously published protocols⁶⁷. Host cells containing the mutagenesis plasmid were prepared as described for PANCE above. Twelve colonies were picked into individual wells of a 96-well plate containing 1 ml of DRM and the appropriate antibiotics. The colonies were resuspended and serially diluted by a factor of ten eight times into DRM. The plate was sealed with a porous sealing film and grown at 37° C. with shaking at 230 RPM for 16-18 hours. Wells containing dilutions with OD₆₀₀ ˜0.3-0.4 were combined and used to inoculate a chemostat containing 100 ml DRM. The chemostat was grown to OD₆₀₀ ˜0.4-0.8, then continuously diluted with fresh DRM at a rate of 1-1.5 chemostat volumes/h to keep the cell density constant. The chemostat was maintained at a volume of 80-100 ml.

Prior to selection phage (SP) infection, lagoons were filled with 15 ml with culture from the chemostat and pre-induced with 10 mM arabinose for at least 1 hour. Lagoons were infected with SP at a starting titer of 108 pfu/ml. To increase stringency, the lagoon dilution rates increased over time as indicated in FIG. 9. During the evolution, samples (800 μL) of the SP were collected from the lagoon waste lines at the indicated times. Samples were centrifuged at 6,000 g for 10 minutes, and the supernatant was stored at 4° C. Titers of SP samples were determined by plaque assays using S2060 cells transformed with pJC175e⁴⁶. The sequences of individual plaques were determined as by PCR with the AB1793/AB1396 primer pair, as described above in the Bacteriophage Cloning methods. Mutation analyses were performed using Mutato. Mutato is available as a Docker image at hub.docker.com/r/araguram/mutato.

High-Throughput Sequencing Analysis of Plasmid Editing in E. coli

To generate the base-editor-expressing cells, 20 μL of 10-beta Electrocompetent E. coli (New England BioLabs) were distributed into a 16-well Nucleocuvette strip. Target plasmid and editor plasmid (0.5 μL each at 100-200 ng/μL) were added to each well, and E. coli were electroporated with a 4D-Nucleofector System (Lonza) using bacterial program X-5. Electroporated cells were immediately recovered in 120 μL SOC media (New England BioLabs) by shaking at 230 rpm at 37° C. for 1 hour. Cells were plated on the appropriate selection antibiotics, along with 100 mM glucose to suppress expression of the base editor, and incubated at 37° C. overnight. The following morning, single colonies were inoculated into 300 μL DRM with antibiotic in separate wells of a 96-well plates (n=4 replicates per condition). The plate was sealed with a porous sealing film and cells were grown to saturation by shaking at 37° C. (˜8 hours). Saturated cultures were diluted 1:50 into 1 ml of DRM with antibiotics and grown to mid-log phase (˜1.5 hours). To induce expression of the base editor, arabinose was added to the cultures (30 mM final concentration), and cells were grown overnight at 37° C. with shaking at 230 RPM. After 16 hours, cells were resuspended by mixing with a multichannel pipet, and 60 μL from each well was transferred into a PCR plate. Cells were lysed by boiling at 95° C. for 8 minutes using a thermal cycler (BioRad). Cell lysates were stored at −20° C. prior to analysis.

For high-throughput sequencing, 1 μL E. coli lysate was used as a PCR template for amplification with the Nextera HTS primers to install adapters as indicated in Table 2. Phusion HiFi polymerase (New England BioLabs) was used for amplification. Barcoding and high-throughput sequencing was performed as described for mammalian cell experiments below.

### General Mammalian Cell Culture

HEK293T (ATCC CRL-3216) cells were purchased from ATCC and Dulbecco's modified Eagle's medium (DMEM) plus GlutaMAX (ThermoFisher Scientific) supplemented with 10% (v/v) fetal bovine serum (Gibco, qualified). Cells were incubated, maintained, and cultured at 37° C. with 5% CO2. Cell lines were authenticated by their respective suppliers and tested negative for Mycoplasma. Undifferentiated 129P2/OlaHsd mESCs (males) lines were maintained as previously described¹¹. Briefly, cells were maintained on gelatin-coated plates in mESC medius (Knockout DMEM (life Technologies), 0.55 mM 2-mercaptoethanol (Sidma) and 1×ESGRO LIF (Millipore) 5 nM GEK-3 inhibitor XV, and 500 nM UO123. Cells were incubated, maintained, and cultured at 37° C. with 5% CO₂. Cell lines were authenticated by their respective suppliers and tested negative for Mycoplasma.

### HEK293T Cell Transfection

Cells were seeded at a density of 1.5×10⁴ cells per well on 96-well plates (Corning) 16-24 hours prior to transfection. Transfection conditions were as follows: 0.5 μL Lipofectamine 2000 (Thermo Fisher Scientific), 100 ng of editor plasmid, and 40 ng of guide RNA plasmid were combined and diluted with Opti-MEM reduced serum media (Thermo Fisher Scientific) to a total volume of 12.5 μL and transfected according to the manufacturer's protocol. Cells were transfected at approximately 60-80% confluency.

Genomic DNA Isolation from Mammalian Cell Culture

Following transfection, cells were cultures for 3 days, after which media was removed, cells were washed with 1×PBS solution (100 μL), and genomic DNA was harvested via cell lysis with 50 μL lysis buffer added per well (10 mM Tris-HCl, pH 8.0, 0.05% SDS, 20 μg/ml Proteinase K (New England BioLabs)). The cell lysis mixture was incubated for 1-1.5 hours at 37° C. before being transferred to 96-well PCR plates and enzyme-inactivated for 30 minutes at 80° C. The resulting genomic DNA mixture was stored at −20° C. until analysis.

Generation of Base Editor mRNA from In Vitro Transcription

Base editor mRNA was generated from PCR product amplified from a template plasmid containing an expression vector for the base editor of interest cloned as described previously⁸. PCR product was amplified in a 200 μL total reaction using forward primer IVT-F and reverse primer IVT-R (Table 4), purified using the QIAquick PCR Purification Kit (Qiagen), and eluted in 50 μL nuclease-free H2O. In vitro transcription was done using the HiScribe T7—High-Yield RNA synthesis Kit (New England BioLabs) according to the manufacturer's protocols but with full substitution of N1-methyl-pseudouridine (TriLink Biotechnologies) in place of uridine and co-transcriptional capping with CleanCap AG (TriLink Biotechnologies). mRNA isolation was performed by lithium chloride precipitation. Briefly, for 160 μL IVT reaction, 0.5 volumes of 7.5 M lithium chloride was added (240 μL final volume) and mixed by pipetting. Following incubation of the mixture at 4° C. for 20 minutes, samples were centrifuged at 15,000×g for 20 minutes. Supernatant was discarded, and pellet was resuspended with 400 μL ice-cold 70% ethanol. Mixture was centrifuged at 4° C. for 15 minutes, and supernatant was discarded gain. The resulting pellet was air-dried at room temperature for 5 minutes and then resuspended in 100-200 μL nuclease-free H2O. An aliquot of the re-suspension was diluted 5-fold for quantification by nanodrop. Samples were normalized to 2 μg/μL and stored at 80° C.

Electroporation of TadCBE mRNA and sgRNA into T Cells or HSCs

Buffy coats from de-identified human donors (n=4) were obtained from Memorial Blood Centers (St. Paul, MN) and peripheral blood mononuclear cells were isolated using Lymphoprep and SepMate tubes (STEMCELL Technologies, Vancouver, Canada). From these, CD4+ cells were purified with the EasySep Human CD4+ T Cell Isolation Kit (STEMCELL Technologies, Vancouver, Canada) followed by activation with Dynabeads™ Human T-Expander CD3/CD28 beads (Thermo Fisher Scientific, Waltham, MA) and culture in X-VIVO™ 15 Serum-free Hematopoietic Cell Medium (Lonza, Basel, Switzerland) that contained: 5% AB human serum (Valley Biomedical, Winchester, VA), GlutaMAX (Gibco, Waltham, MA), N-acetyl-cysteine (Sigma Aldrich, St. Louis, MO), 50 U/ml penicillin and 50 μg/ml streptomycin (Gibco, Waltham, MA), and 300 IU/ml IL-2. At 72 hours, the beads were removed and 300,000 T-cells electroporated with 2 μg of candidate base editor mRNA and 100 pmol sgRNA (Synthego, Redwood City, CA) using the Neon electroporation system with 10-μl tips (ThermoFisher, Waltham, MA). Sequences of the chemically synthesized guide RNAs used in this experiment are listed in Table 5.

CD34+ cells without any identifying donor information were procured from the Core Center for Excellence in Hematology at the Fred Hutchinson Cancer Research Center (Seattle, WA) and cultured in SFEM II media (STEMCELL Technologies, Vancouver, Canada) containing: 50 U/ml penicillin and 50 μg/ml streptomycin (Gibco, Waltham, MA), 100 ng/ml each of recombinant human thrombopoietin, stem cell factor (TPO; BioLegend, San Diego, CA), Flt-3 ligand, and IL-6 (Peprotech, Cranbury, NJ) and 0.75 μM StemRegenin1 and 500 nM UM729 (STEMCELL Technologies, Vancouver, Canada). At 48 hours after thawing (n=3 donors), 2 μg of editor mRNA and 100 pmol of sgRNA were electroporated into 200,000 HSC using the Amaxa (Lonza, Basel, Switzerland) 4D-Nucleofector protocol for P3 Primary Cell Line 4D Nucleofector Kit in Nucleovette strips, program DZ-100. Sequences of the chemically synthesized guide RNAs in this experiment are listed in Table 5.

At 72 hours after gene transfer, cell pellets were harvested for DNA using the QuickExtract™ DNA Extraction Solution (Madison, WI). PCR amplification for Illumina sequencing was performed using Phusion U Multiplex PCR Master Mix (Thermo Fisher Scientific, Waltham, MA) under the following conditions: 30 s at 98° C.; 30-35 cycles at 98° C. for 10 seconds, 64° C. for 30 seconds, and at 72° C. for 20 seconds; and a final of 72° C. for 5 minutes.

### High-Throughput DNA Sequencing of Genomic DNA Samples

High-throughput sequencing of genomic DNA from mammalian cell lines was performed as previously described⁴. Primers for PCR amplification of target genomic sites are listed in Tables 2A-2E. Sequences of the target amplicons are listed in Tables 2A-2E. DNA concentrations were quantified using a Qubit dsDNA High Sensitivity Assay Kit (Thermo Fisher Scientific) by qPCR with the KAPA DNA quantification kit (Roche) prior to sequencing on an Illumina MiSeq instrument according to the manufacturer's protocol.

### Analysis of Cas-Independent RNA Editing

RNA off-target editing analysis was performed as previously described¹⁵. Briefly, parallel plates of HEK293T cells were transfected with 250 ng of plasmid encoding editors and 83 ng of EMX1 guide RNA plasmid as described above. One plate was used to evaluate on-target genomic DNA editing at the EMX1 locus as described above. The other plate was used for RNA editing analysis as follows: Cells were lysed 48 hours after transfection using the RNeasy kit (Qiagen) following the manufacturer instructions. Briefly, Culture medium was removed and cells were washed with PBS before lysis in RLT Plus Buffer (QIAGEN). Cells were transferred to a DNA eliminator column. Ethanol was added to the flowthrough which was transferred to an RNeasy spin column. Samples were washed with RW1, then on-column DNA digestion was carried out with RNase-Free DNase in RDD buffer (QIAGEN®). Samples were then washed with RW1 buffer followed by a wash with RPE buffer. RNA was eluted in 45 μl nuclease-free water and 2 μl RNaseOUT (Thermo Fisher Scientific) was added to each sample.

Complementary DNA was generated with the SuperScript IV First-Strand Synthesis Kit (Thermo Fisher Scientific) according to the manufacturer's instructions. The OligodT primer was annealed to RNA by heating at 65° C. then cooling on ice for 1 minute. Reverse transcription reactions were prepared and added to the annealing mixtures. No-reverse transcriptase controls were included as a control for gDNA contamination. Reactions were incubated at 50° C. for 10 minutes, 80° C. for 10 minutes, then cooled on ice for one minute. The optional RNA degradation with RNaseH was carried out to increase the efficiency of cDNA amplification. The first PCR of amplicon sequencing was conducted with 1 μl of each cDNA sample; the remaining sequencing protocol is identical to that used for DNA sequencing. Primers used for first PCRs are listed in Table 3.

### Library Analysis of TadCBE Editing Outcomes

Base editor plasmids were constructed by cloning the new editor sequences into the previously described p2T-CMV-AID-BE4max-BlastR plasmid¹¹. Undifferentiated 129P2/OlaHsd mESCs (males) lines containing the previously reported 10,683-member “comprehensive 12kChar” library¹¹. Cells were thawed and maintained on 15-cm plates as previously described¹¹. To integrate the base editor plasmid into the cell lines containing the integrated library, cells were transfected with Tol2 transposase plasmid using Lipofectamine 3000 and selected with blasticidin starting the day after transfection for 4 days before harvesting. An average coverage of 300× per library cassette was maintained throughout. Two biological replicates were performed per base editor. Genomic DNA was collected from cells 4 days after antibiotic selection (5 days after base editor transfection). For library samples, 20 μg of genomic DNA was used for each sample for PCR1 amplification and sequencing and an average sequencing depth of 2,800× per target was maintained. PCR1 was performed to amplify the endogenous locus or library cassette using the primers specified in Table 8. PCR2 was performed to add full-length Illumina sequencing adapters using the NEBNext Index Primer sets1 and 2 (New England Biolabs). All PCR reactions were performed using NEBNext Ultra II Q5 Master Mix. Extension time for all PCR reactions was extended to 2 min per cycle to prevent PCR amplification bias. Samples were quantified by Tape Station (Agilent), pooled, and quantified using a KAPA Library Quantification kit (Roche) before sequencing. Library sequencing was performed on an Illumina NextSeq with paired end reads (94 forward; 56 reverse).

Data processing and analysis were performed with Python 3.9. Library samples were demultiplexed for each editor/replicate with bcl2fastq2 (Illumina), with all lanes merged. To assign each paired-end read to a library member, any reads below Q28 in the target sites and sgRNA spacer sequence were discarded. Candidate target sites were then nominated with locality-sensitive hashing using tiled 6-mers across the target site. Any reads where the sgRNA spacer sequenced did not match a candidate target site were filtered out. Finally, each target site was genotyped by performing Needleman-Wunsch alignment (scoring parameters: match=1, mismatch=−1, gap open=−5, gap extend=0, start gap=0).

Prior to further data analysis, two sources of noise in the sequencing data were considered. First, the expansion of the mESC cell line harboring the genomically integrated libraries could lead to the stochastic amplification of errors present in the initial cell library after selection (so-called “batch effects”). Second, next-generation sequencing on Illumina systems can occasionally misassign reads. To minimize both error sources, only A-to-G, C-to-T, C-to-G, and C-to-A mutations within the −9 through 20 base editing window, were considered.

Potential batch effects were investigated in the mutation data by comparing the frequencies of each mutation at each position within the window with one-way ANOVA. Encouragingly, there were no batch-specific mutations within the window that were outside of the range of statistical noise (at a Bonferroni-corrected significance level of 0.005).

Finally, reads that were likely due to Illumina sequencing noise were filtered out. It was considered that mutations due to rare base editing outcomes would likely still be present across both replicates of the library, even if their presence in each replicate were below the threshold that would be traditionally considered noise. Therefore, the likelihood that each mutation at each position would be observed in the corresponding number of reads was computed in both replicates based on a Bernoulli distribution with a rate parameter of 10⁻³ (Q30). All mutations that were less than 5% likely to be due to sequencing noise were kept.

For position-wise editing efficiency analyses, the number of reads containing each mutation was combined between replicates and divided by the total number of reads observed for each given library member. Replicates were combined in this way (rather than, for example, averaging the frequencies for each replicate) because it is the maximum-likelihood estimate of the rate parameter of a hypothetical Bernoulli distribution that describes the base editing efficiency at a given position.

In the present analyses, the “average editing efficiency” was defined across the library as the average fraction of (noise-filtered, batch-combined) reads containing the specified editing outcome. To define selectivity for cytosine over adenine deamination, the average cytosine editing efficiency and the average adenine editing efficiency was first computed at positions within the ≥30% editing window across all members of the library. The geometric mean of the selectivity was then computed at each position to obtain a conservative estimate of the “overall” selectivity of each editor. Since a given position can only contain either a cytosine or an adenine, the true selectivity in a given scenario will depend on the positions of the respective bases.

To generate sequence motifs of the context preferences of these editors, the editing fraction was first transformed with a stabilized logit function:

$$log ( \frac{x + ϵ}{1 + ϵ - x} ) ,$$

where ∈ is a small constant that stabilizes the function behavior for inputs close to 0 or 1. For these purposes, ∈=0.001 was used, as this is a conservative estimate of the noise due to Illumina sequencing. A random train/test split (80:20, respectively) was then performed and a ridge regression was trained with α=10⁻⁵ to generate weights that were visualized in a sequence logo.

To evaluate the fold-changes in C·G-to-T·A and A·T-to-C·G conversion efficiency upon inclusion of the V106W mutation in TadCBEd, total least-squares (TLS) regression was performed on the (noise-filtered, batch-corrected) efficiency of installing the specified edit with each editor. TLS was performed, rather than ordinary least-squares, because the calculation involved a relationship between two measured variables (as opposed to the dependence of one variable on another, independent variable). The average fold-decrease was defined as the reciprocal of the regression weight (where x is TadCBEd and y is TadCBEd-V106W).

### Analysis of HTS Data for DNA Sequencing and Targeted Amplicon Sequencing

Individual high-throughput sequencing data sets were demultiplexed using the MiSeq Reporter (Illumina). Subsequent demultiplexed sequencing reads were analyzed using CRISPResso2⁷⁷ as described previously¹⁵. All editing values are representative of n=3 independent biological replicates, with mean±SEM shown.

### Example 6. Broad Characterization of TadCBEs on 10,683 Genomic Target Sites in Mammalian Cells

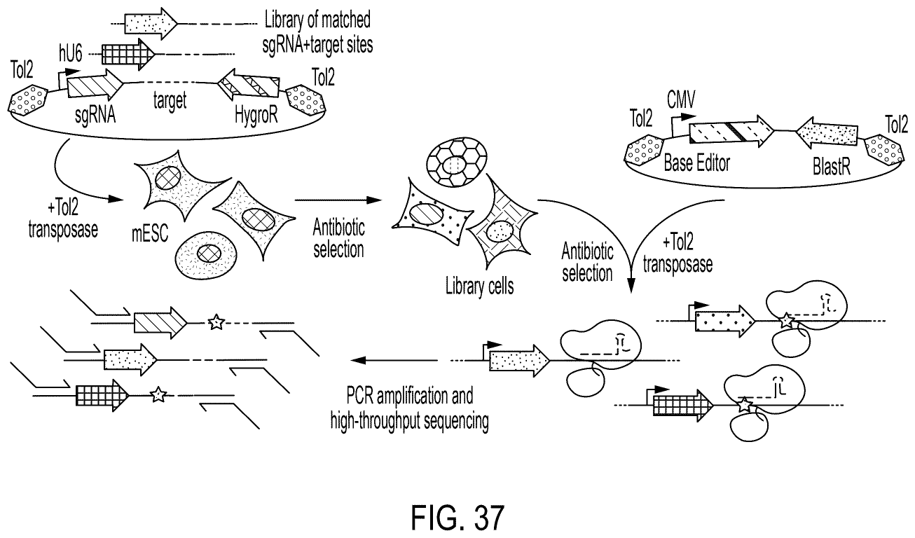

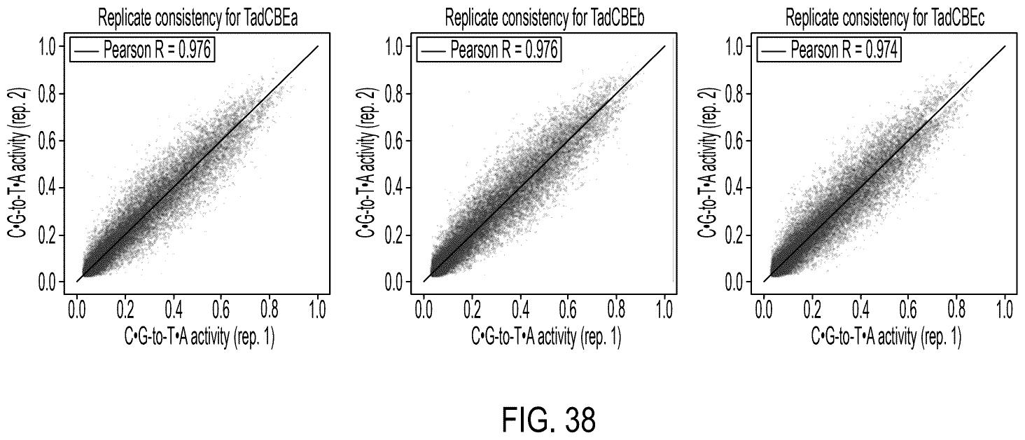

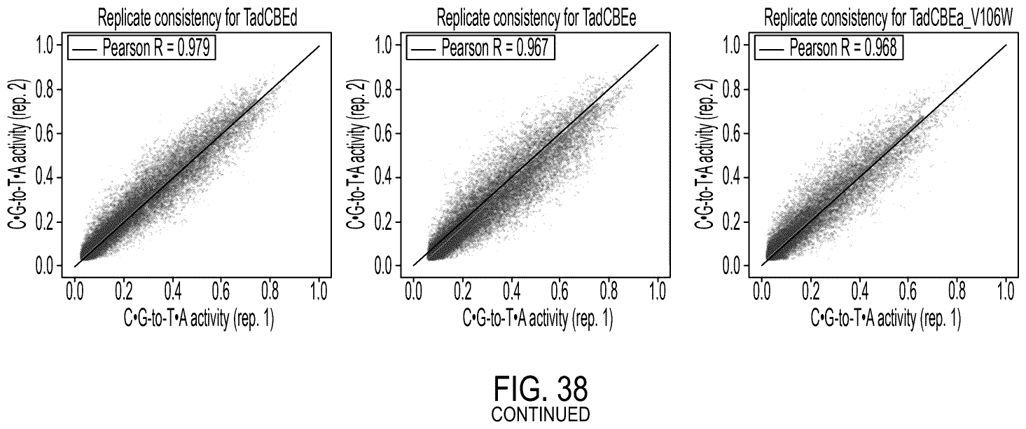

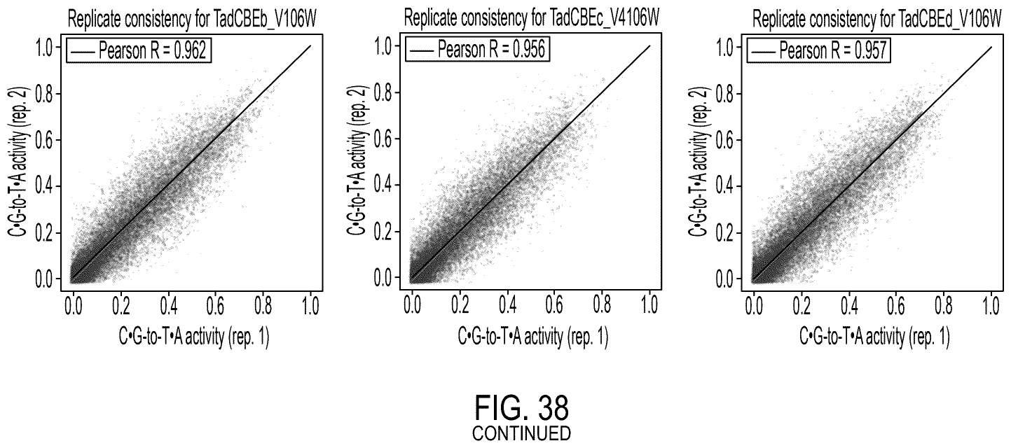

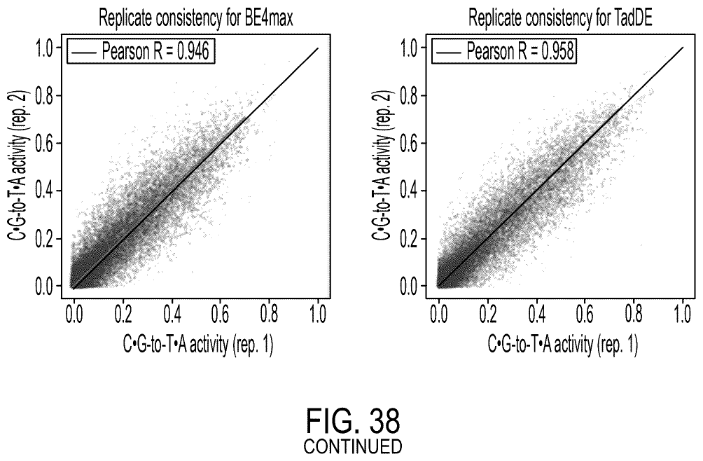

TadCBE activity can vary substantially by target site (FIG. 3). To comprehensively characterize the activity of TadCBEs across a wide range of sites in mammalian cells, high-throughput analysis of base editing outcomes was performed for TadCBE variants using a previously reported ‘comprehensive context library’ of 10,683 paired sgRNA and target sites integrated into a mouse embryonic stem cell line (mESCs, FIG. 37)¹¹. These libraries include target sites with all possible 6-mers surrounding a substrate A or C nucleotide at protospacer position 6 and all possible 5-mers across positions −1 to 13 (counting the position immediately upstream of the protospacer as position 0) with minimal sequence bias¹¹. Base editing conditions were optimized allow differences between base editors to be detected. An average cell coverage of ≥300× per library member throughout the course of the experiment and an average sequencing depth of ≥2,800× per target was maintained, which enabled the detection of editing outcomes with high sensitivity. Two biological replicates were collected per base editor for TadCBEa-e, V106W variants of TadCBEa-d, and TadDE, as well as BE4max as a reference¹¹, and validated that the library assay data have strong consistency between biological replicates (FIG. 38).

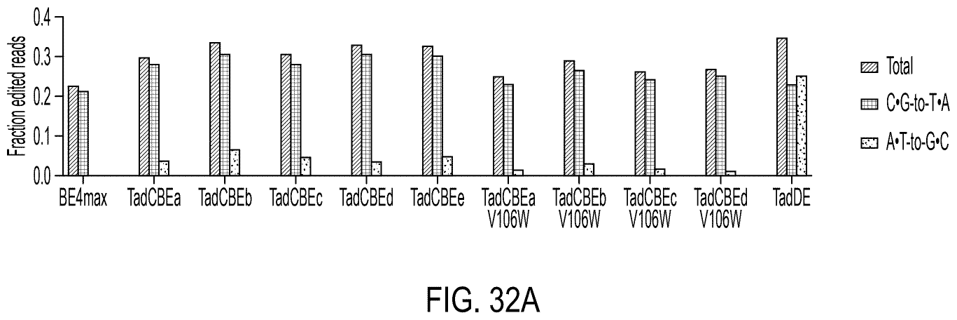

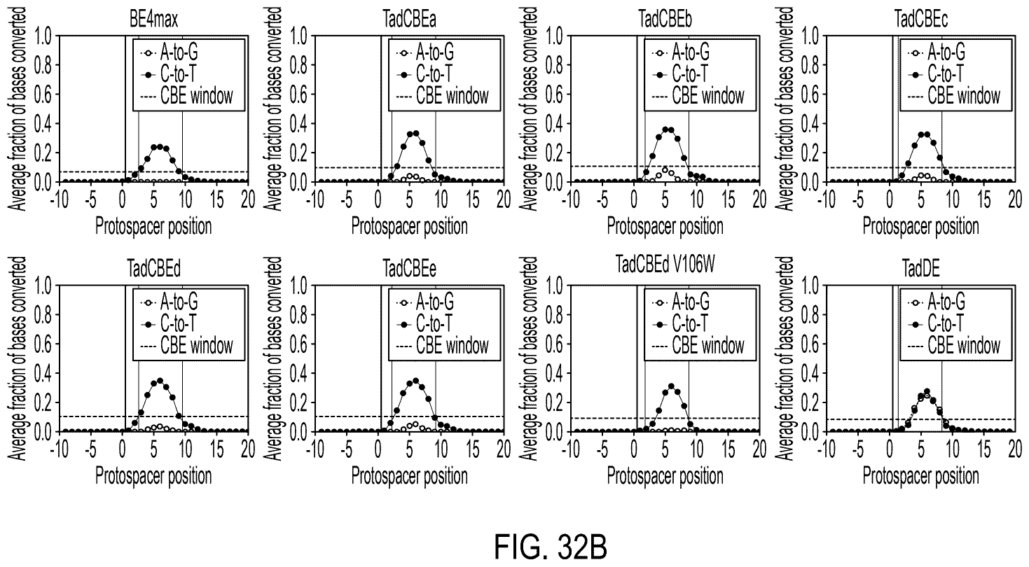

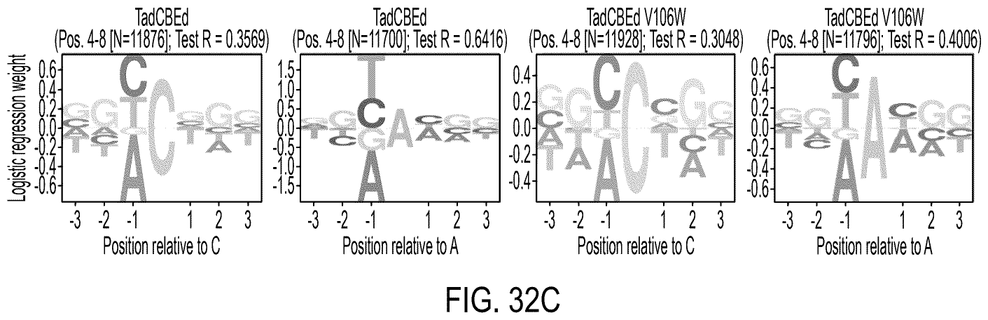

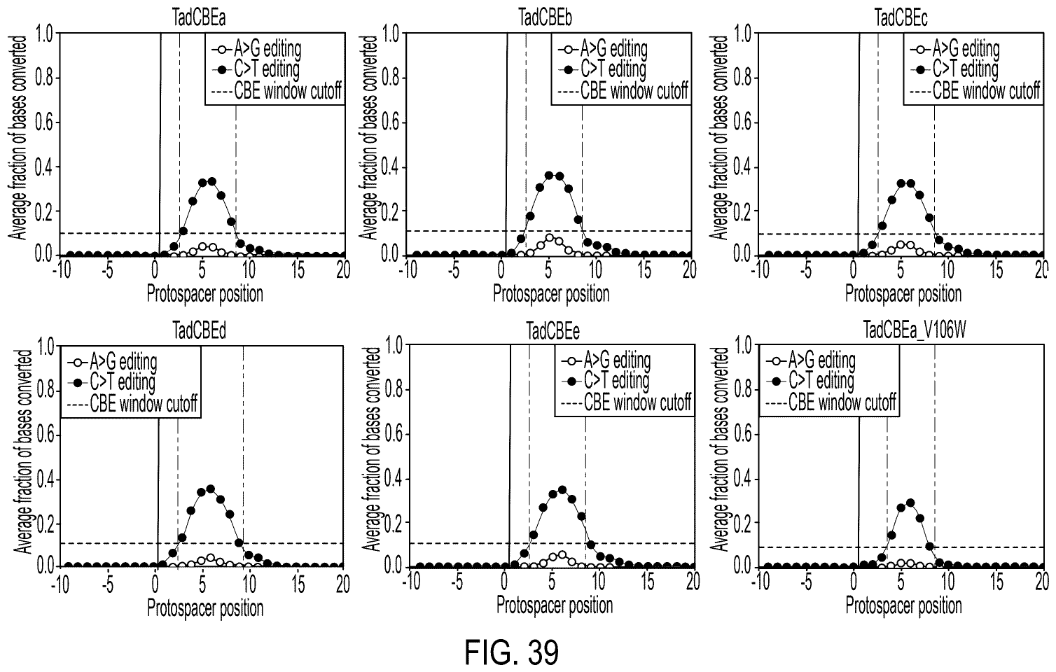

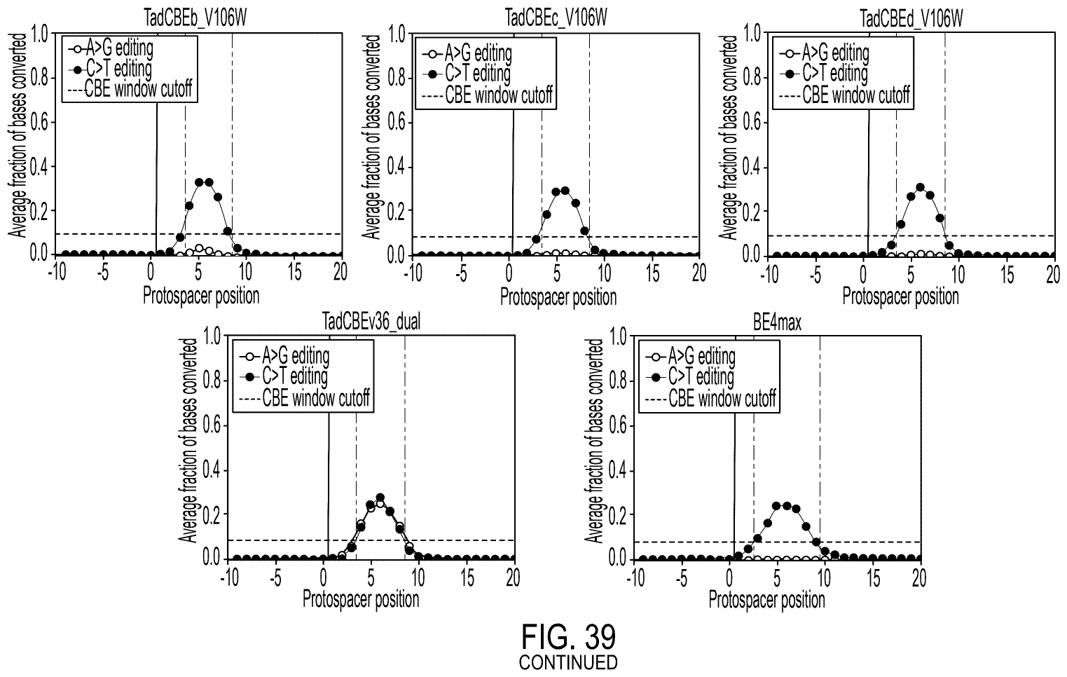

The resulting library data was used to quantify editing activity and C·G-to-T·A selectivity for each TadCBE (FIG. 32A). Across the 10,638 integrated target sites, all TadCBE and TadCBE-V106W variants edited with greater average efficiency (28%-31% of reads on average with any C·G-to-T·A editing) than BE4max (21%) (FIG. 32A)¹¹. The editing windows were then characterized, which were defined as positions within the protospacer that averaged ≥30% of the peak average editing efficiency (FIG. 32B, FIG. 39). TadCBE editing is generally centered around protospacer position 6. The most active variant, TadCBEd, has a similar editing window (protospacer positions 3-9) to that of BE4max (positions 3-9), while the remaining TadCBEs and V106W-TadCBEs have slightly narrower windows (positions 3-8, FIG. 32B, FIG. 39).

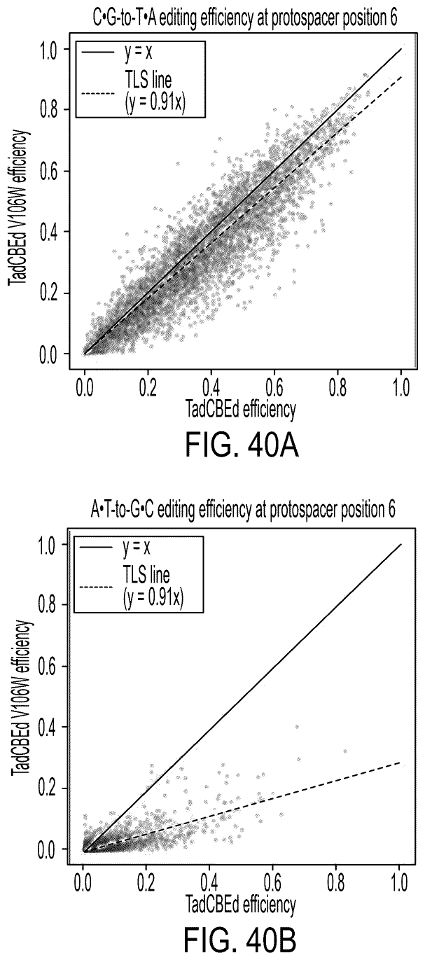

TadCBE selectivity for cytosine editing over adenine editing varied by base editor. Among the canonical TadCBEs (without V106W), TadCBEd showed the highest C·G-to-T·A selectivity, with a geometric mean of the ratio of C·G-to-T·A vs A·T-to-G·C editing at each position in its editing window of 26.8 (Table 6). Notably, the addition of V106W substantially improved C·G-to-T·A selectivity for all TadCBE variants (TadCBEd V106W selectivity=47.8), while minimally affecting base editing activity at the maximally edited position (FIGS. 32A-32B, FIGS. 40A-40B). For example, the addition of V106W to TadCBEd reduced peak editing among the library targets from 35% to 31%.

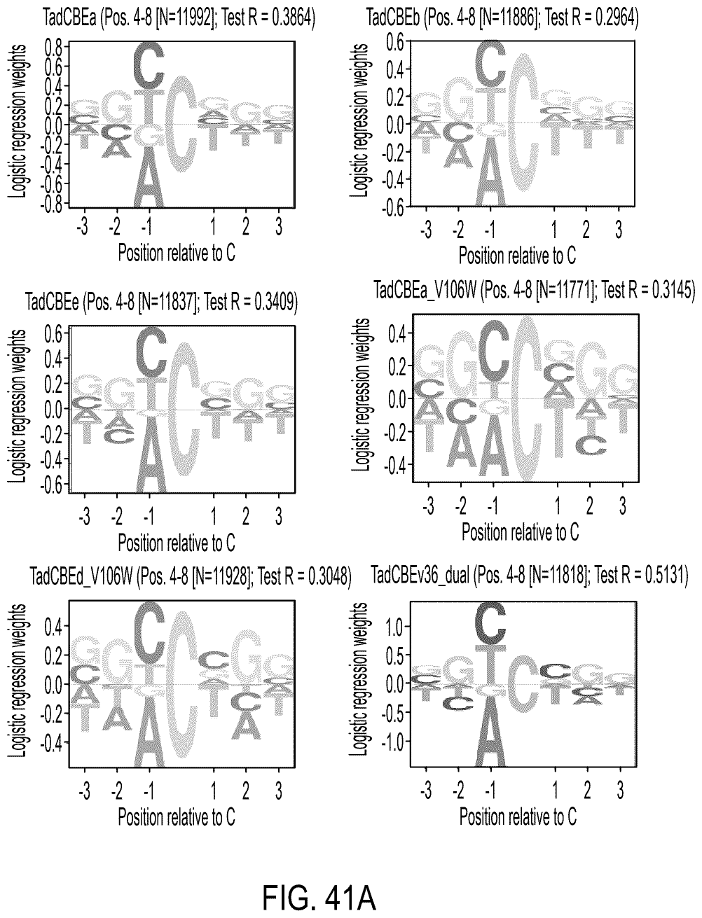

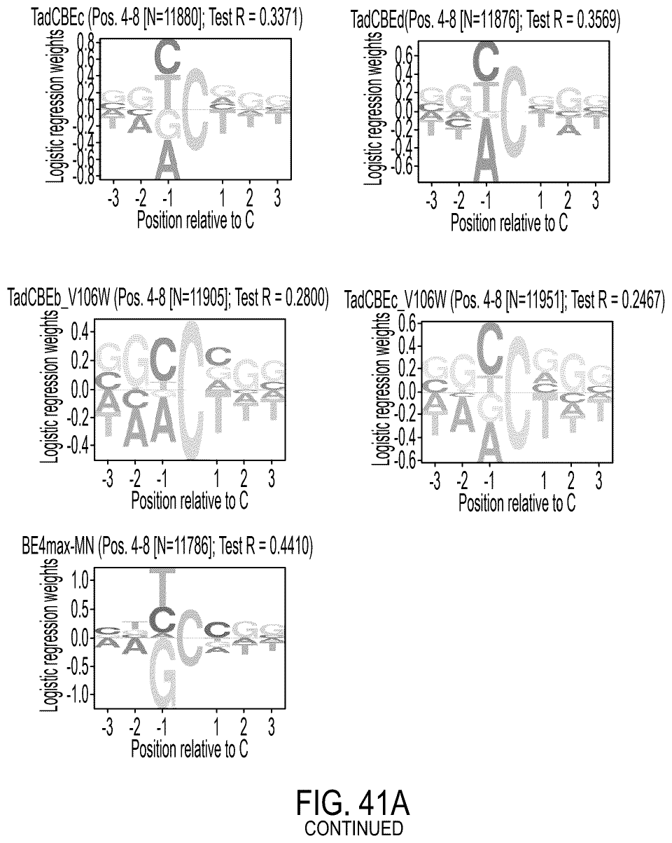

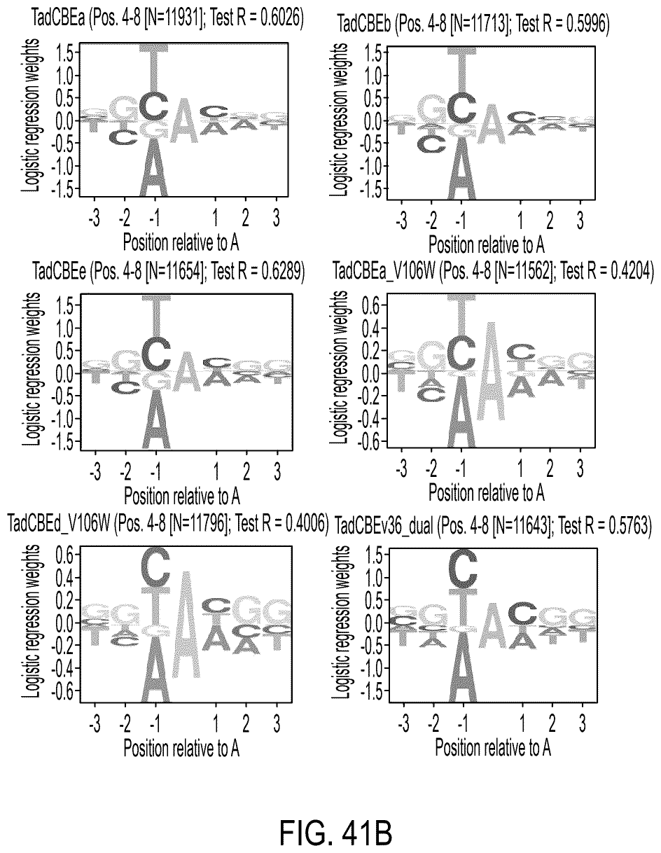

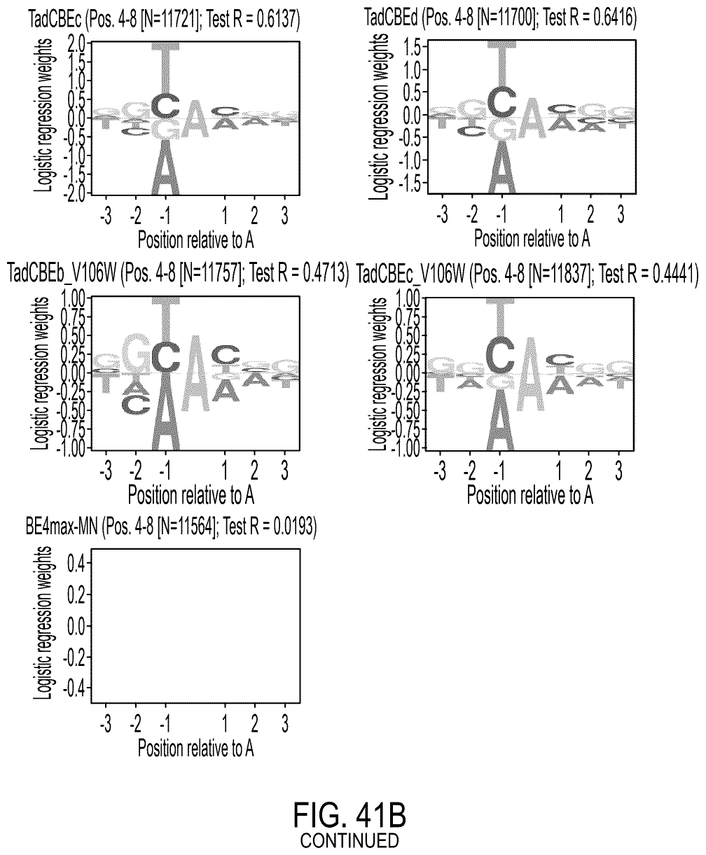

Consistent with the discrete target site examples shown above, C·G-to-T·A selectivity of TadCBEs varied by target site across the comprehensive context library. Adenine base editing was observed in 3.4-6.6% of reads (on average) for TadCBEs and 1.0-2.7% of reads for V106W-TadCBEs across all target sites in the comprehensive context library (FIG. 32A). Sequence motifs were generated by performing regression on the editing efficiencies to determine the sequence characteristics that affect cytosine and adenine deamination (FIG. 32C, FIGS. 41A and 41B). TadCBEs have similar sequence context preferences to their ancestor, ABE7.10, favoring editing of cytosine and adenine bases preceded by 5′ Y (Y=T/C), while disfavoring 5′ A¹¹. When performing bystander adenine base editing, TadCBEs retain the sequence context preference of ABE7.10 (favoring 5′ YAY and disfavoring 5′ AAA). However, TadCBEs instead slightly disfavor 5′ ACT. The difference in 3′ preference may be due to differences in substrate positioning required to achieve altered selectivity, since interactions with adjacent bases could alter placement of the target cytidine in the active site (FIGS. 10A-10C). Notably, target sequence dependence is stronger for adenine base editing (TadCBEd Test R: 0.64) compared to cytosine base editing (TadCBEd Test R: 0.36).

TadDE performs very similar levels of adenine and cytosine base editing (ABE:CBE ratio=1.1) and has similar sequence context dependence to TadCBEs (FIGS. 32A and 32B, FIGS. 41A and 41B, Table 6). TadDE is highly efficient, editing 35% of the reads on average in the library experiment (FIG. 32A). The probability of observing A·T-to-G·C editing given that C·G-to-T·A editing is observed is 0.62 for TadDE, compared to 0.04 for TadCBEd-V106W, the most selective TadCBE variant (Table 6). The high activity, promiscuity, and small size of TadDE makes it a promising tool for concurrent A·T-to-G·C and C·G-to-T·A editing.

Collectively, these data show that TadCBEs have greater cytosine deamination activity than conventional narrow-window CBEs. Furthermore, the introduction of V106W in the deaminase domain reduces residual A·T-to-G·C editing activity while minimally impacting C·G-to-T·A editing for all TadCBEs in this experiment. Overall, TadCBEd enables the greatest cytosine deamination activity with high C·G-to-T·A selectivity, which is further improved by addition of V106W.

### Example 7. TadCBE Compatibility with Cas9 Orthologs and Editing Window Characterization

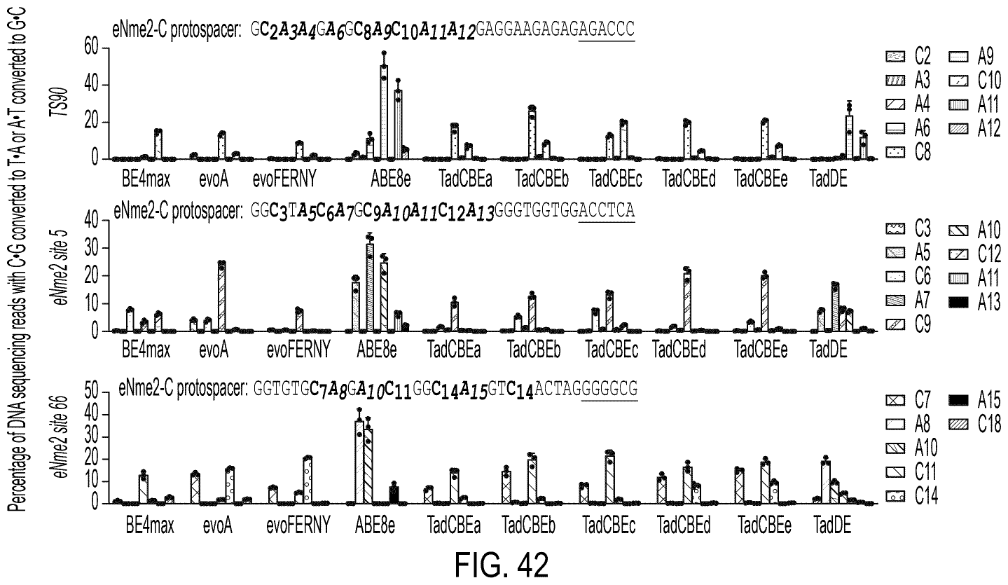

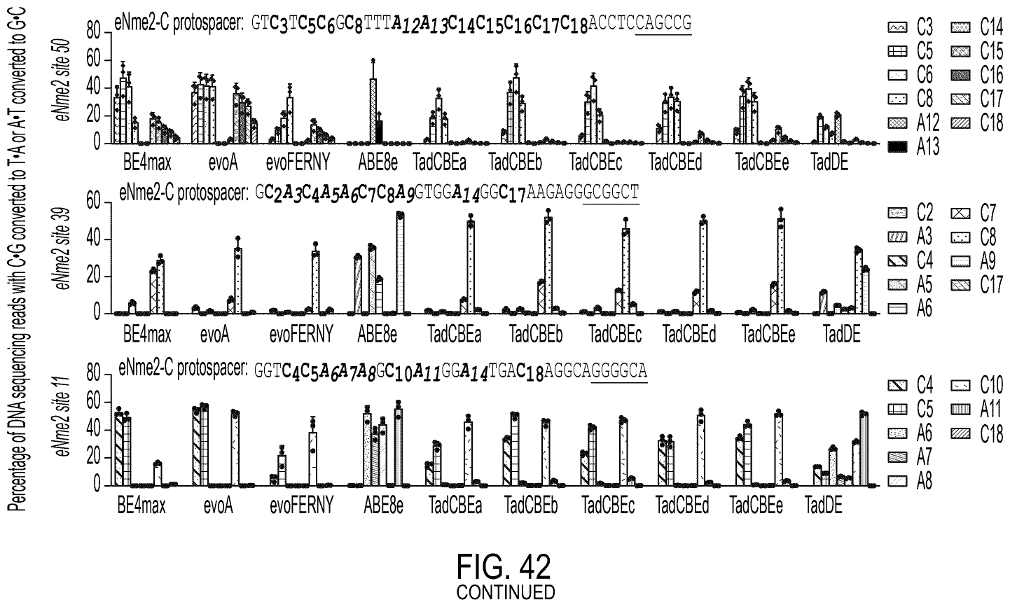

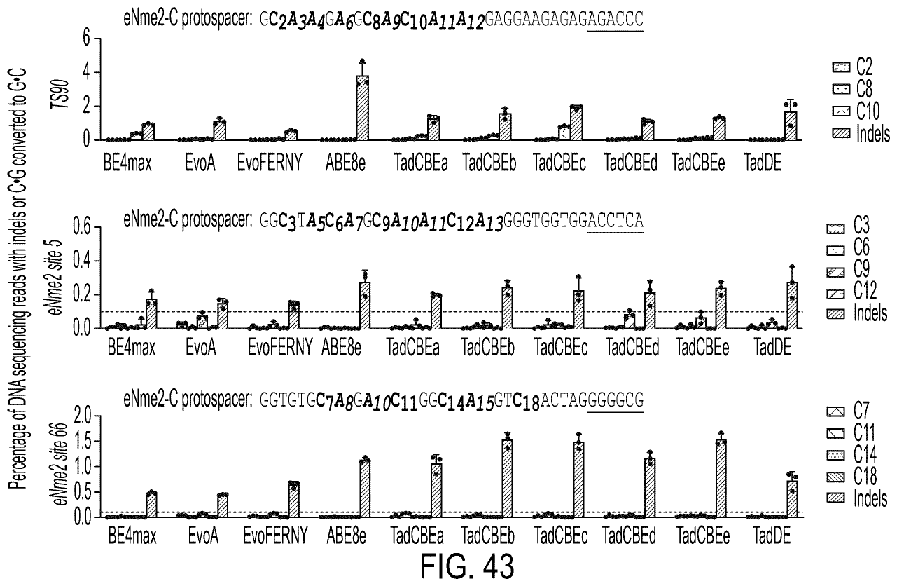

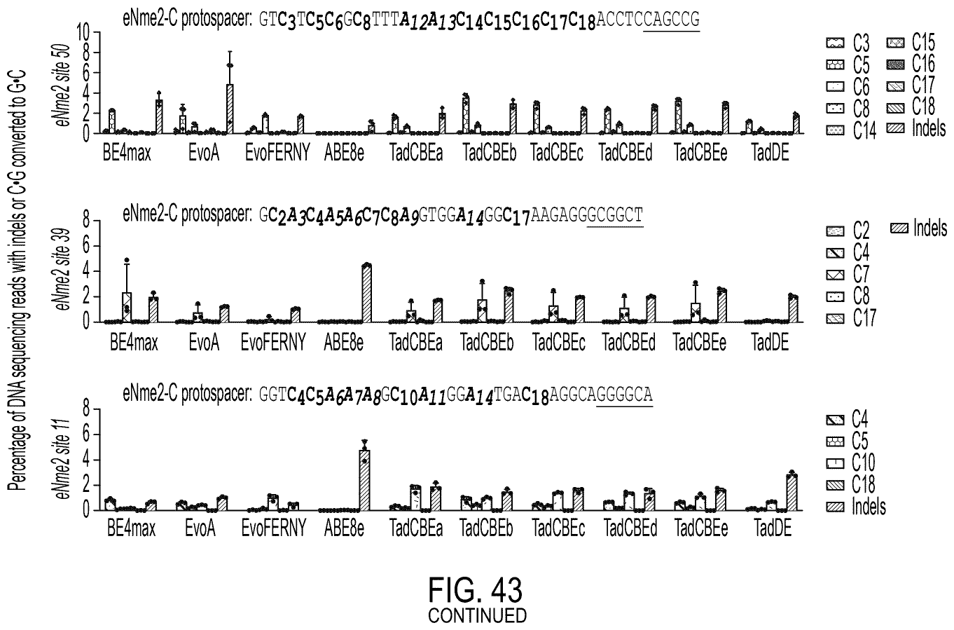

The use of Cas9 orthologs with diverse PAM requirements expands the targetable sequence space of base editors. To test if TadCBEs are compatible with Cas9 homologs beyond Streptococcus pyogenes Cas9, TadCBE variants with PACE-evolved variants of Nme2Cas9 from Neisseria meningitidis were constructed that broadened the scope of accessible PAMs beyond the canonical NGG PAM of SpCas9⁶². Nme2Cas9 variants that access a wide range of single-pyrimidine PAM sites as nucleases or as base editors were recently evolved⁶³. Fusions of TadA-CDs were generated with an eNme2-C variant nickase (SEQ ID NO: 353) (PAM=N₄CN)) and two UGI domains, co-transfected the resulting eNme2-C-TadCBEs with a guide RNA plasmid, and examined base editing at six genomic loci in HEK293T cells. Across all tested sites, the peak editing efficiency of TadCBEs was comparable to that of APOBEC1, evoFERNY, and evoAPOBEC1 (FIGS. 42-43). Although C·G-to-T·A editing exceeded 50% at some sites, residual A·T-to-G·C editing never exceeded 5.3% at any of the six eNme2 target sites tested. TadCBEs thus exhibited robust activity and selectivity with eNme2 Cas9.

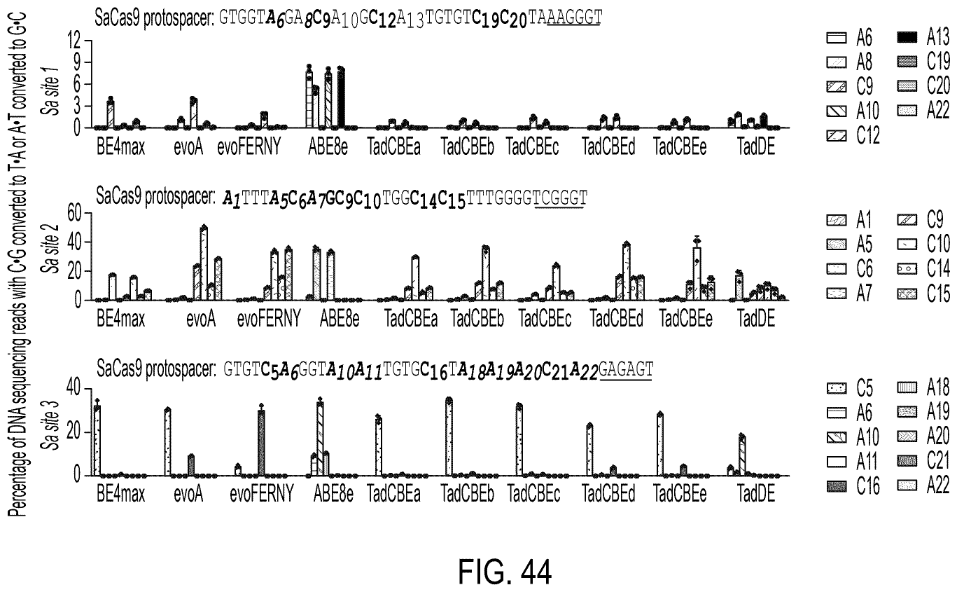

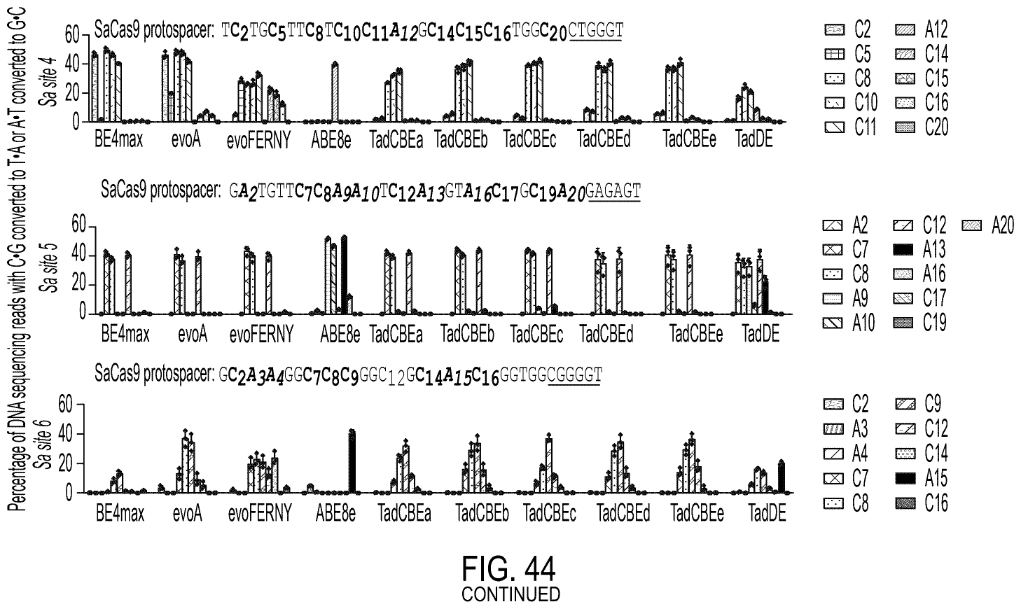

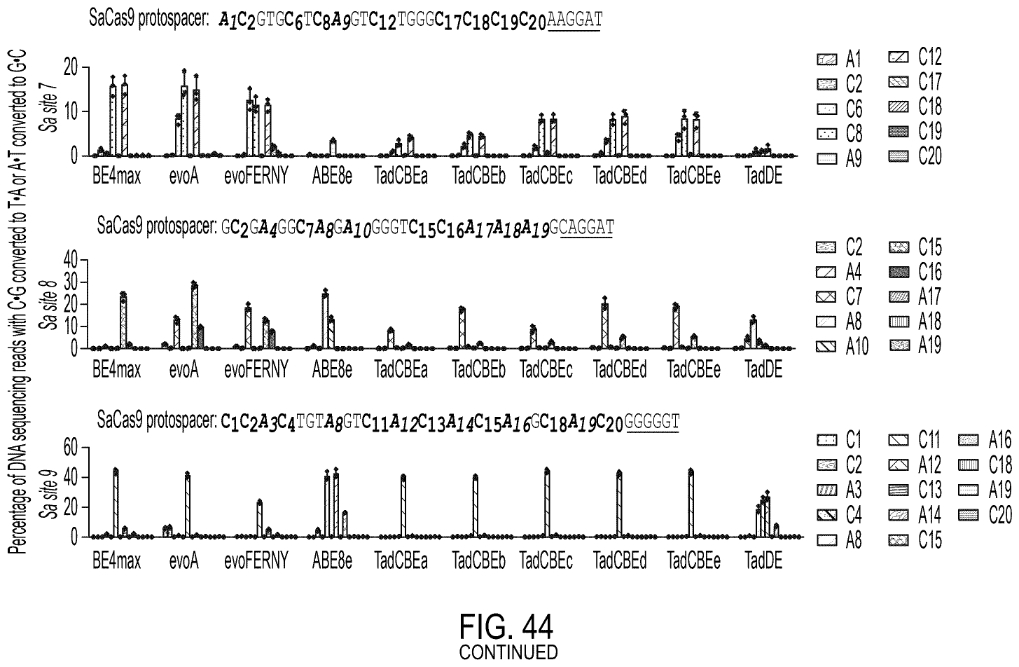

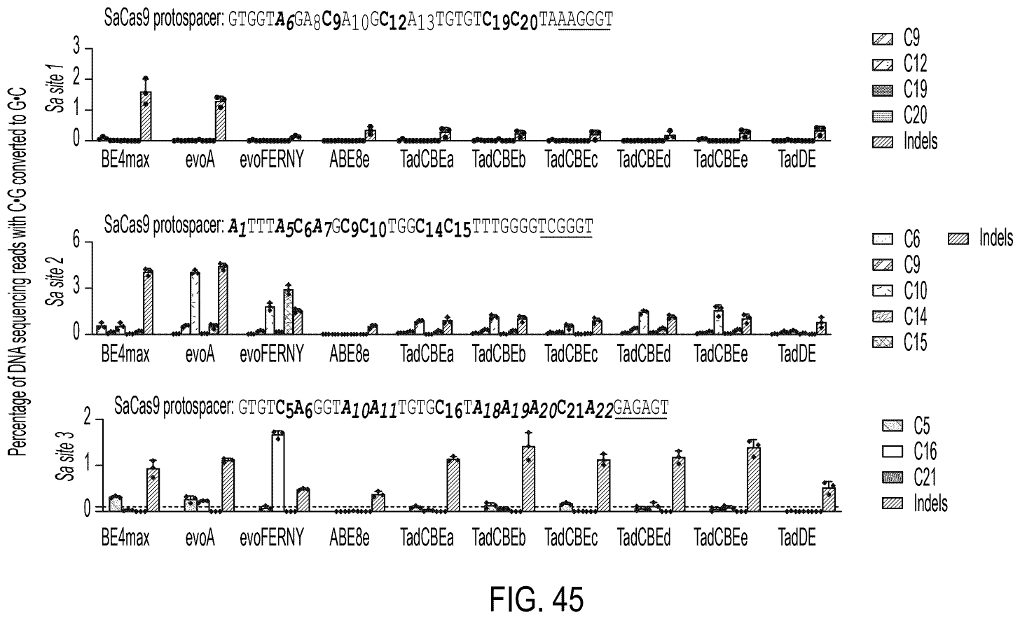

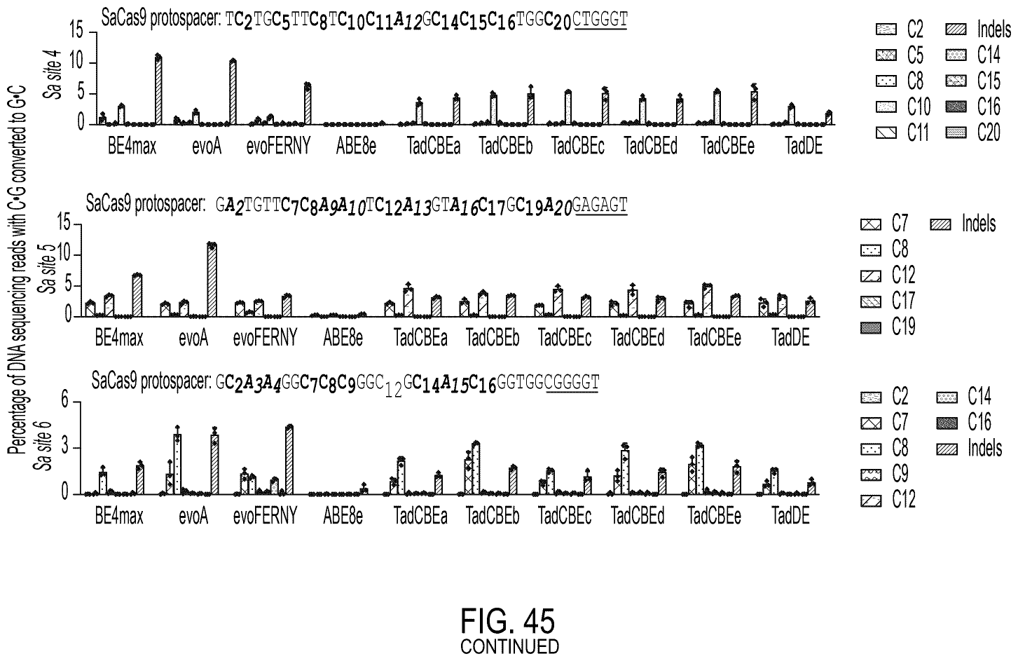

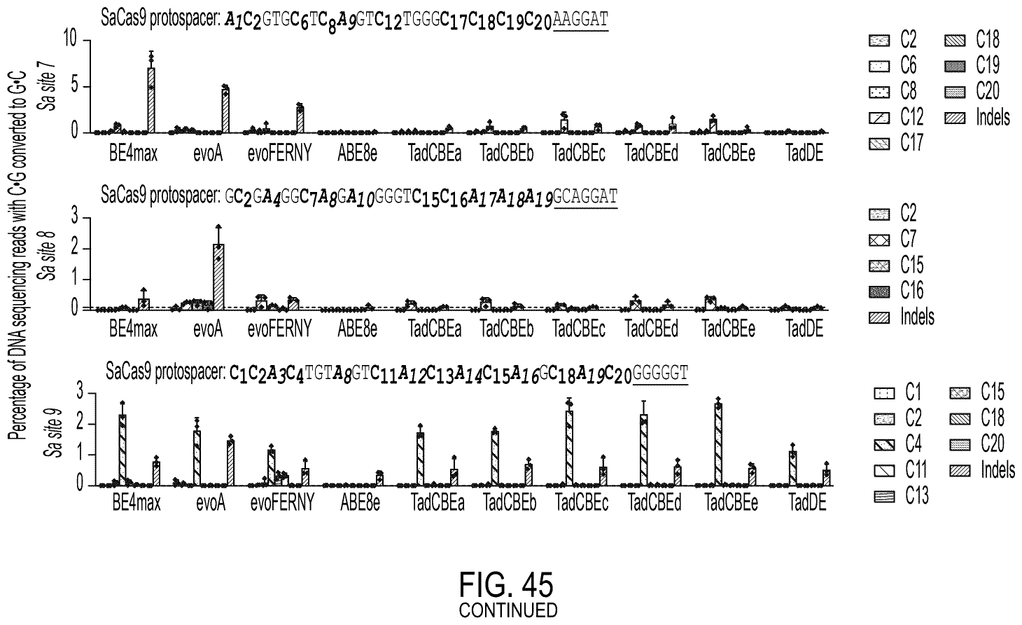

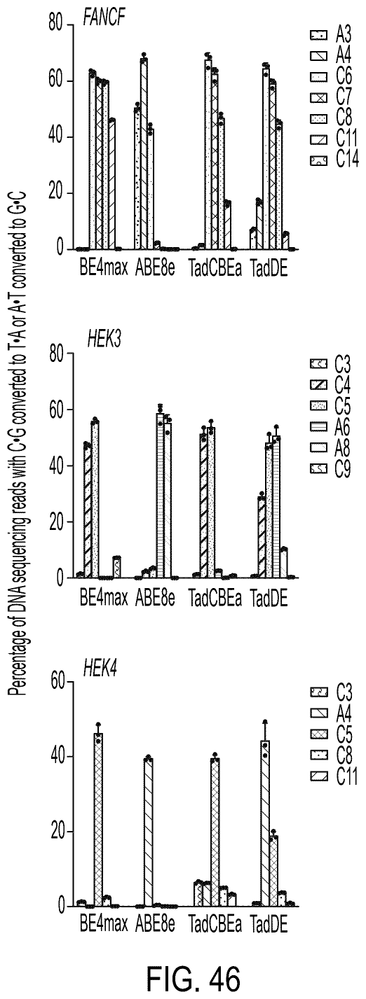

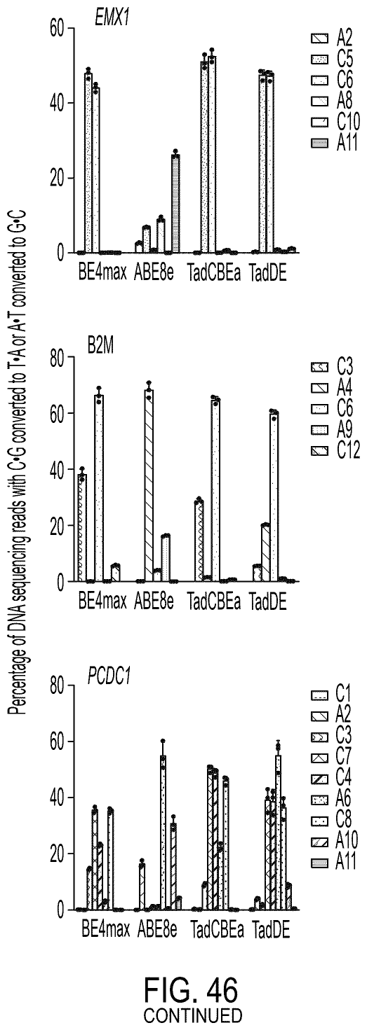

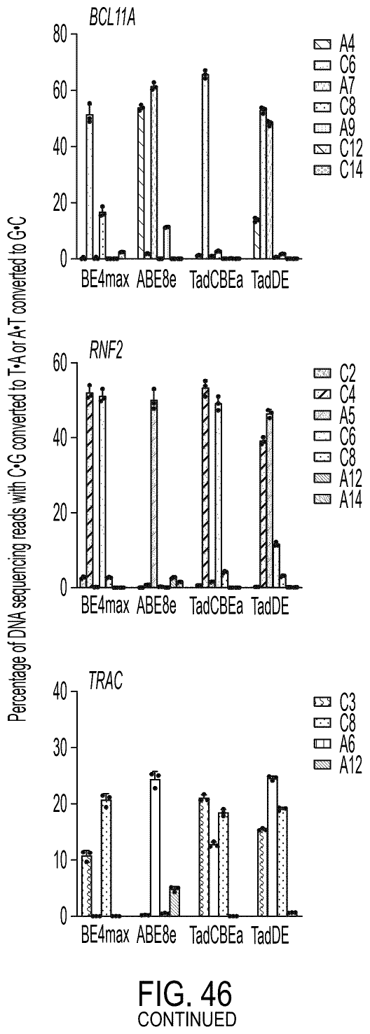

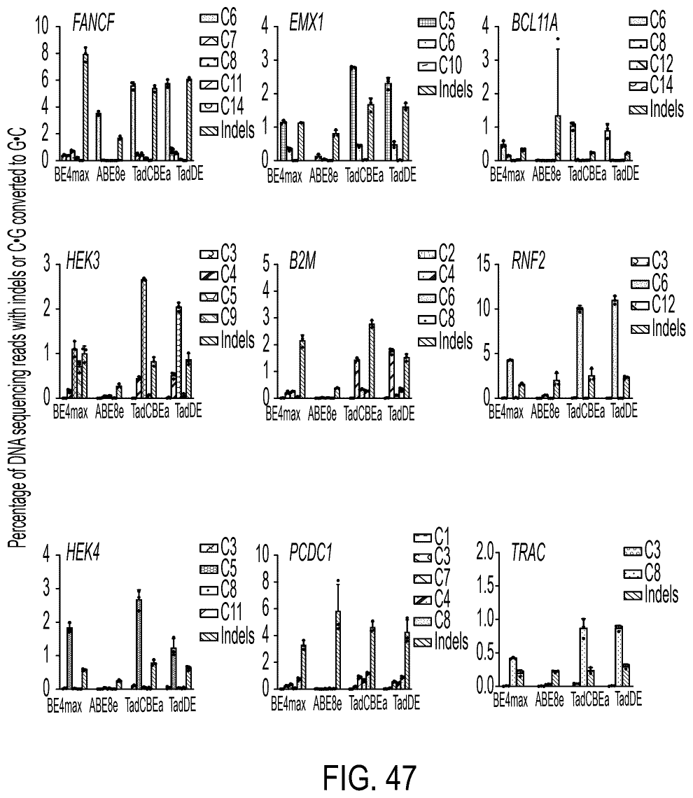

TadCBEs were next tested with Staphylococcus aureus Cas9 (SaCas9) in the BE4max architecture⁶⁴. SaCas9 (1053 amino acids) is smaller than SpCas9 (1368 amino acids) and recognizes a different PAM sequence (PAM=NNGRRT). It was found that TadCBEs using SaCas9 have robust C·G-to-T·A editing across 9 sites (4.1-44%) with less than 5.5% A·T-to-G·C at any site (FIGS. 44-45). These observations suggest potential compatibility with other Cas proteins that together with SpCas9, eNme2-C Cas9, and SaCas9 may offer access to a variety of PAM sequences for versatile targeting of TadCBEs. It was additionally found that TadDE performed both A·T-to-G·C and C·G-to-T·A editing with SpCas9, eNme2-C Cas9, and SaCas9 in mammalian cells at sites where TadCBEs were selective, suggesting broad Cas9 compatibility of the dual editor as well (FIGS. 42-47).

TadCBEs exhibit a narrower editing window than BE4max, evoA, and evoFERNY CBEs, while maintaining comparable or higher maximal editing efficiencies (FIG. 42). For example, BE4max and evoA edited Nme50 at protospacer positions 3-18 with 4.2-47% efficiency, while TadCBEa, TadCBEb, and TadCBEc modify only the narrower position 3-8 window with 5-48% efficiency (FIG. 42). The narrower base editing activity window of TadCBEs could arise from a less processive deaminase, since the processive nature of APOBEC family deaminases can catalyze multiple hydrolytic deamination reactions per DNA-binding event⁶⁵. While a wide editing window can be useful for some applications such as targeted gene disruption or base editing screens, the narrower window of TadCBEs should benefit precision editing applications in which modification of only one target base is desirable, particularly when using Cas9 domains that support a wider base editing window^{62,66}. Taken together, the small size of TadCBEs, their compatibility with eNmeCas9 and SaCas9, their more focused editing windows, and their high editing efficiencies and selectivities for cytosine over adenine base editing demonstrate their suitability for a variety of precision cytosine base editing applications.

### Example 8. Development of an Active and Selective Cytosine Base Editor from a TadA Dual Base Editor Using Phage-Assisted Evolution

In this example the use of an active and selective cytosine base editor for stop codon installation at disease-relevant sites is described. To do this, a continuous flow of E. coli host cells with the selection circuit and a mutagenesis plasmid (red) are infected by selection phage encoding a partial deaminase (SP). In the selection circuit, phage propagation is linked with the expression of gIII (P2), which can only be transcribed with active T7 RNA polymerase. T7 RNA polymerase (P3) is fused to a C-terminal degron, and the deaminase must perform C-to-U editing to install a stop codon before the degron, yielding active T7 RNA polymerase. In the event of phage infection, the full deaminase is completed using a split-intein system (P1) and mutations can occur on the deaminase. Beneficial mutations lead to phage propagation and enrichment in the lagoon, while the less-fit phage are unable to propagate and are subsequently washed out by the constant outflow.

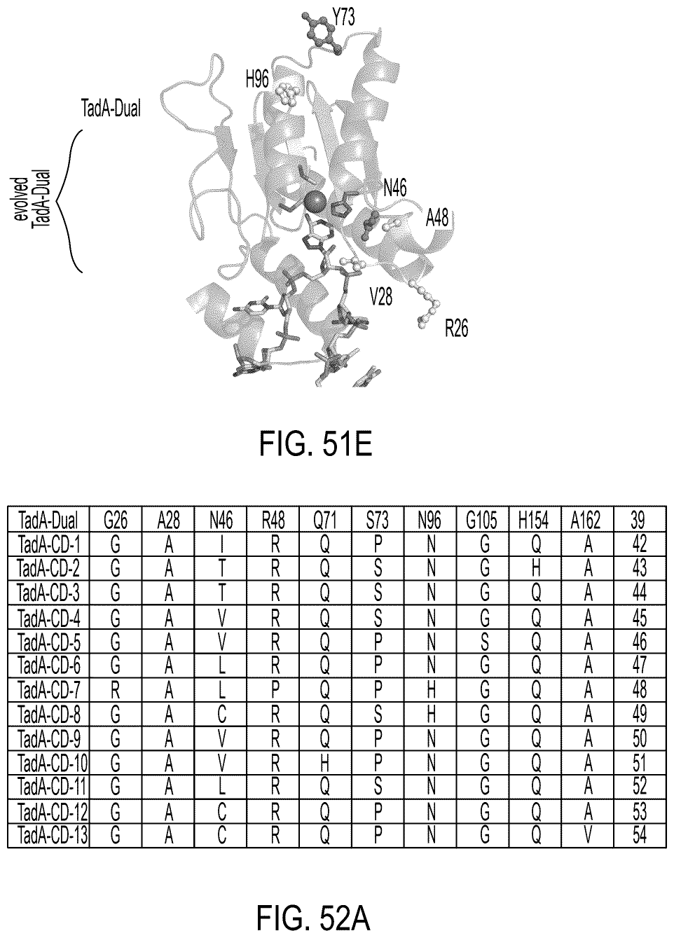

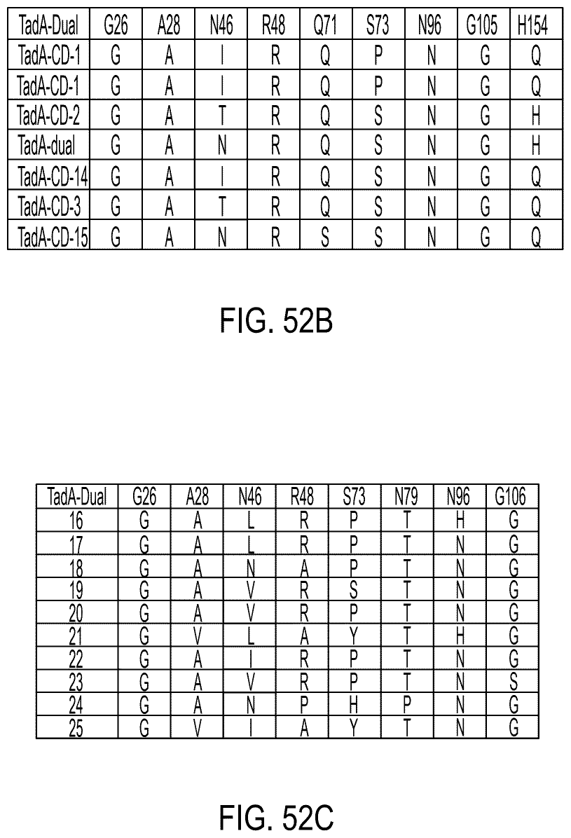

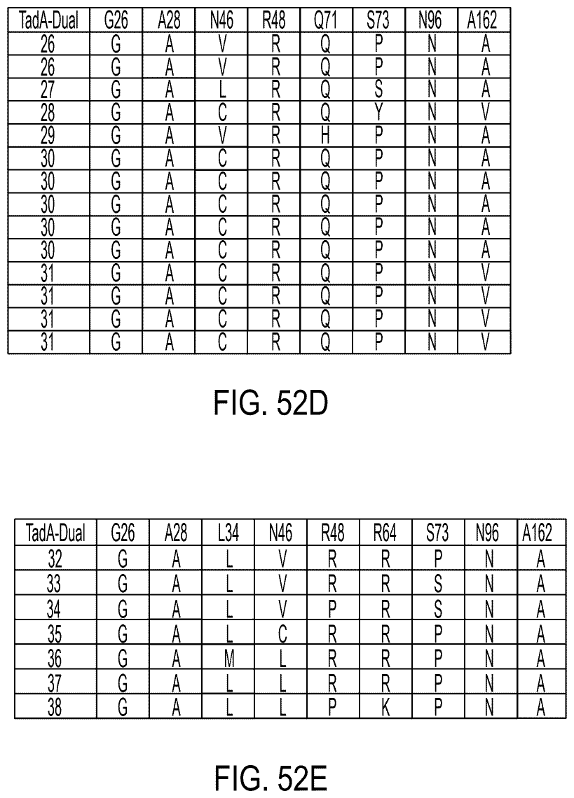

The resulting variant identified a conserved mutation at position N46 in the deaminase, so an NNK library was constructed at position N46, and PANCE was performed on these variants. To increase stringency even further, PACE was performed for >100 hrs on the resulting variants from both PANCEs. Dilution factors are indicated on the right y-axis. Exemplary mutation tables from PANCE and PACE depicting the conserved mutations are shown in FIGS. 52A-52E.

### Example 9. Profiling the Activity and Sequence Context Specificity of TadCBEs in E. coli

A 32-member single-stranded DNA library (IDT oligopools) was designed to contain a target base (A or C) at protospacer positions 6 with the 5′ and 3′ base varied as A, T, C, or G. Each library member contains a unique molecular identifier (UMI) barcode. The single-stranded oligos were amplified for three cycles with the primer pair MN1591/MN1592 with KAPA polymerase using 1.5 nM template in a reaction volume of 200 μl with an annealing temperature of 68° C. and an extension time of 3 min. The PCR product was purified (Qiagen) and assembled into BamHI/EcoRI-digested plasmid MNp553 using Gibson (NEB). Following purification with Glyco-blue (Thermo Fisher), the library was transformed into NEB 10-beta electrocompetent cells. Dilutions of cells were plated immediately to calculate library size, and then the remaining transformants were grown overnight in carbenicillin to select for transformants. The following day, the library plasmid was purified by midiprep (Qiagen).

In parallel, electrocompetent NEB10-beta cells containing the indicated editor plasmid of interested were prepared following grown in DRM to suppress expression. 40 μl of electrocompetent cells containing the editor was then electroporated with 100 ng library plasmid, rescued in 1 ml S.O.C. media for 5 min, diluted in 10 ml DRM, and grown overnight with spectinomycin, carbenicillin, and 30 mM arabinose to induce editor expression. After 16 h growth at 37° C. with shaking at 200 rpm, the plasmids were isolated by miniprep. 1 μl plasmid was used as a template for PCR1 and HTS analysis as indicated below.

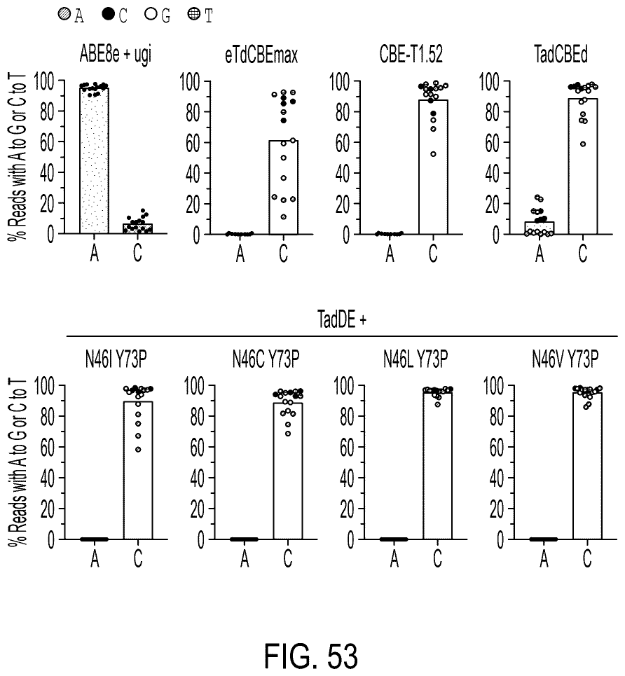

To analyze editing results for the library, sequencing reads demultiplexed using MiSeq Reporter (Illumina) and then sorted into target amplicons using SeqKit. The output was then sequenced using CRISPResso2. The results are shown in FIG. 53.

### Experiment 10. Comparison of the Evolved Active and Selective Cytosine Base Editors with Existing Cytosine Base Editors in Mammalian Cells

TadDE N46 variants along with existing cytosine base editors with SpCas9 nickases in the BE4max architecture were transfected into HEK293T cells with guide RNAs targeting three protospacers. TadDE N46 variants show comparable on-target activity with no residual A-to-G editing. Dots represent individual values from independent biological replicates. PAM sequences are underlined. HEK293T Site 2 is abbreviate HEK2, and HEK293T Site 4 is abbreviated HEK4.

TadDE N46 variants along with existing cytosine base editors with eNme-Cas9 nickases in the BE4max architecture were transfected into HEK293T cells with guide RNAs targeting two protospacers. TadDE N46 variants show higher or comparable on-target activity with no residual A-to-G editing. Dots represent individual values from independent biological replicates. PAM sequences are underlined.

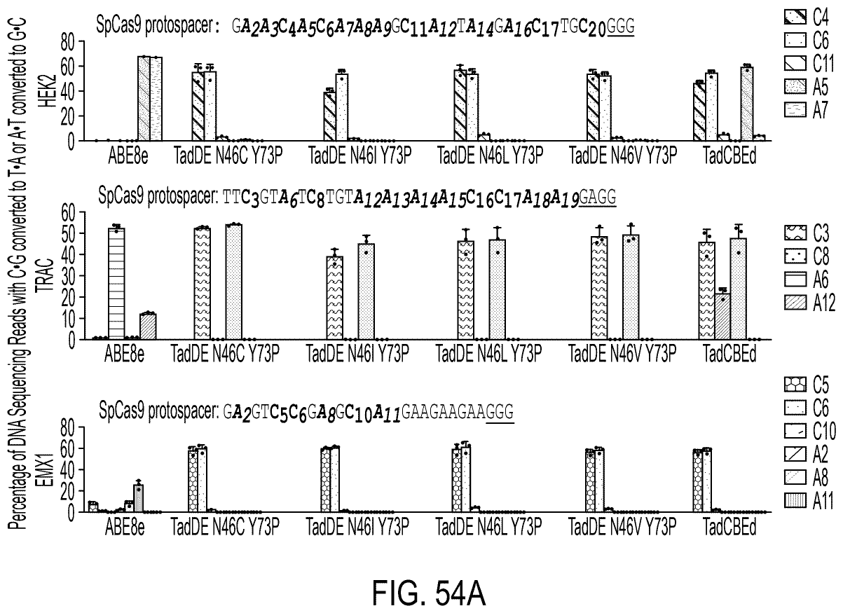

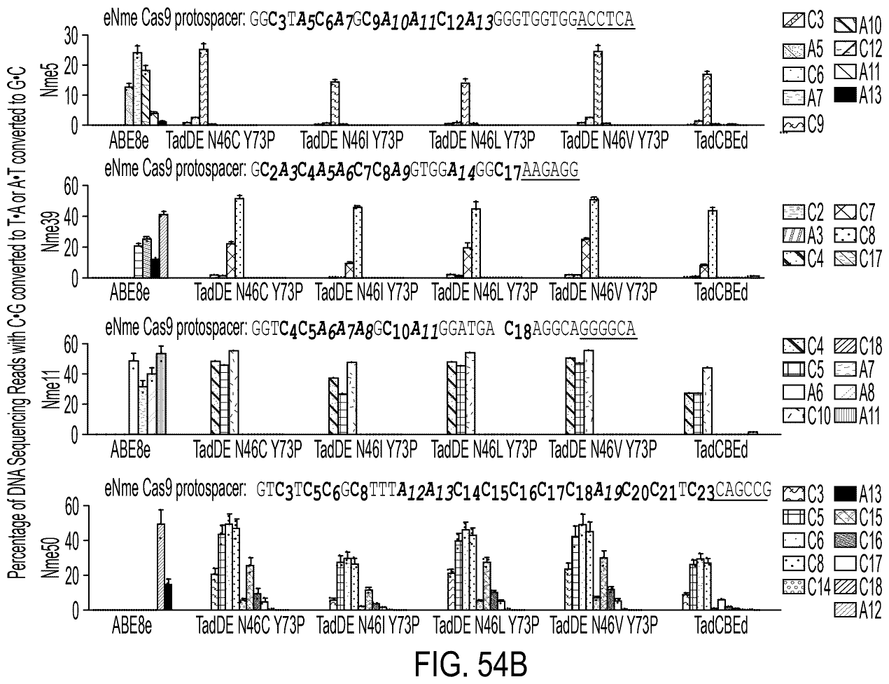

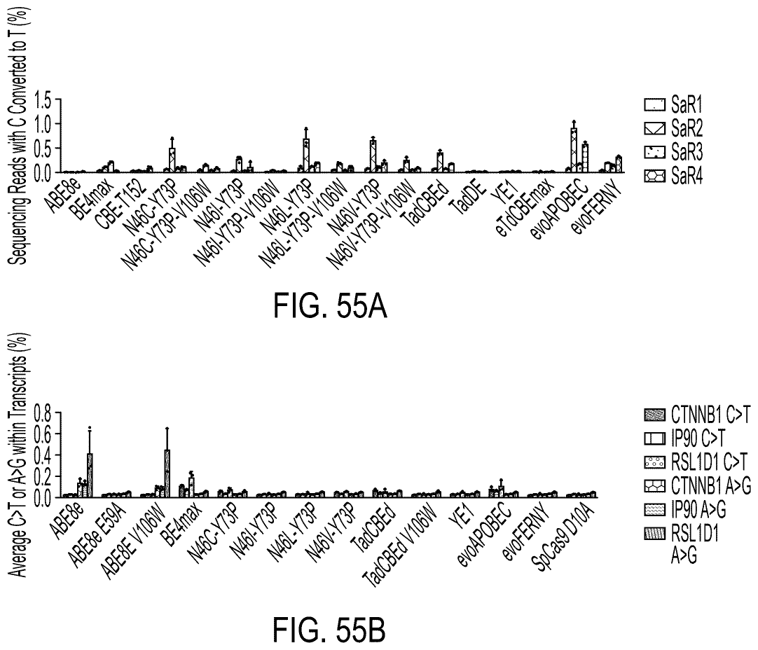

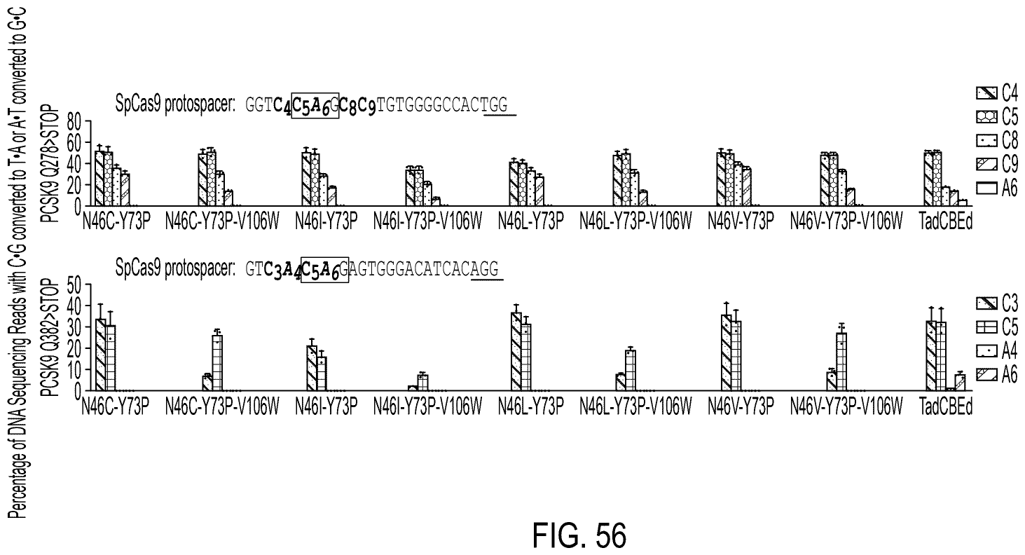

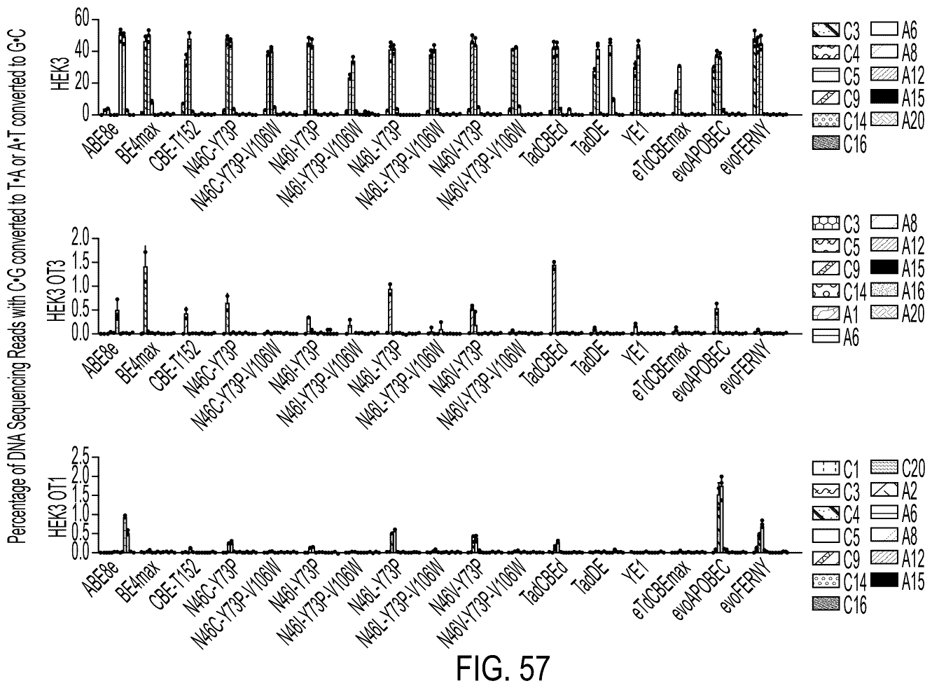

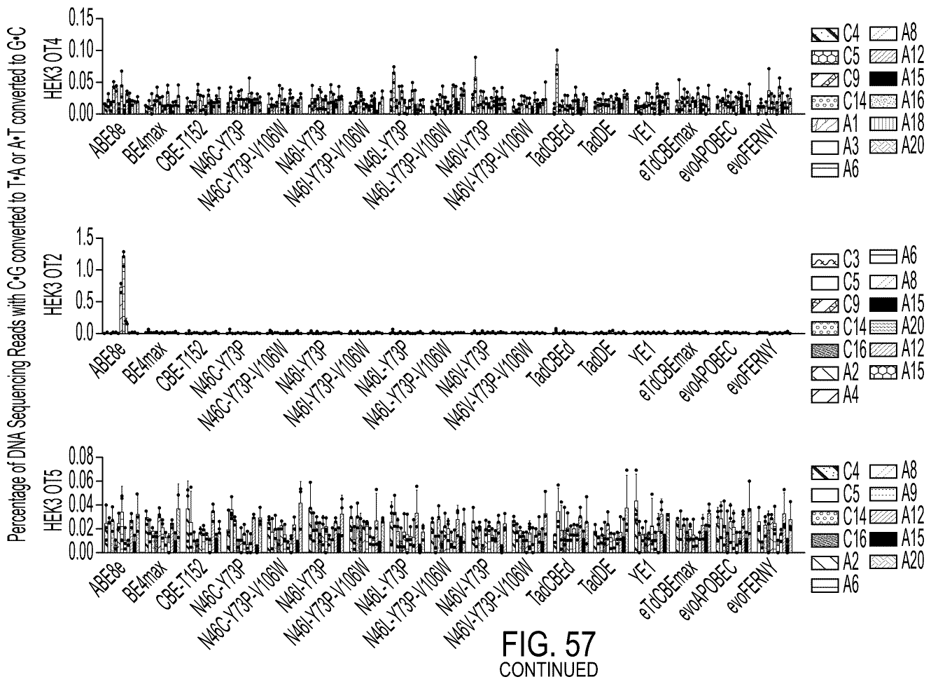

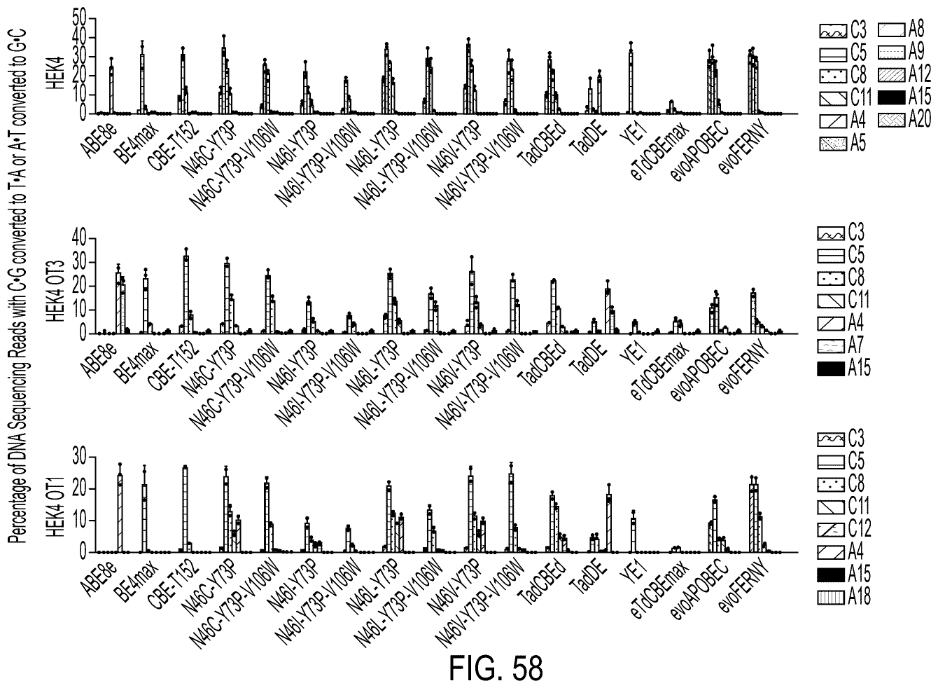

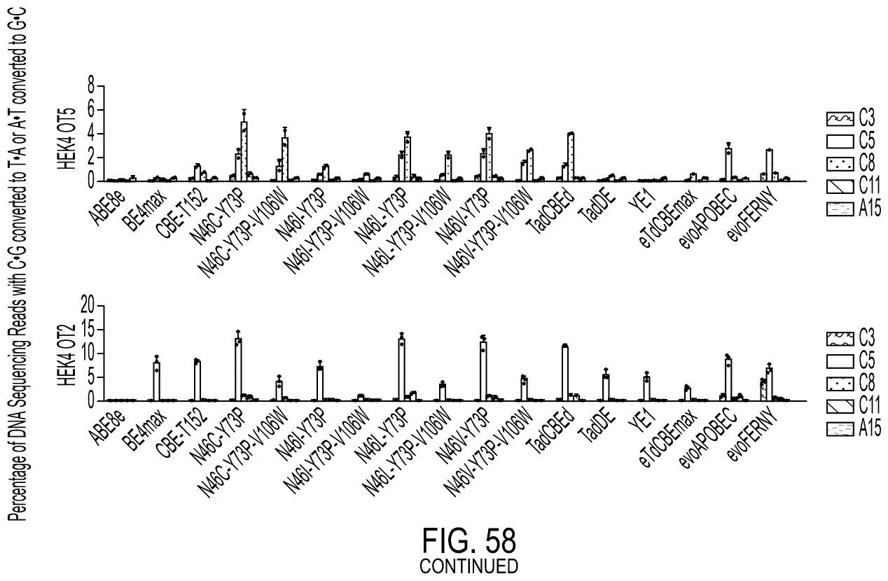

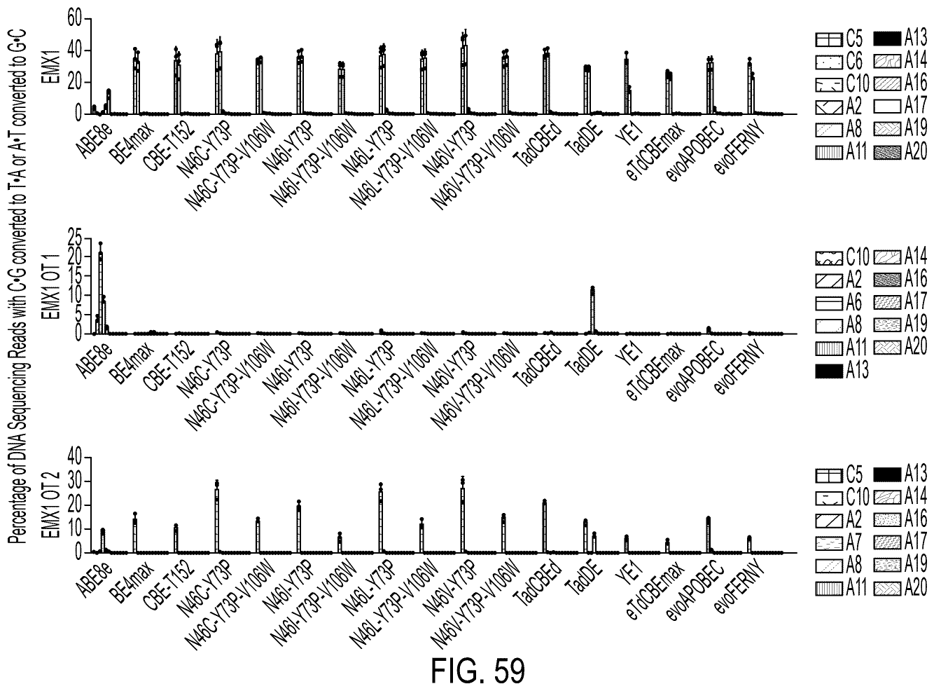

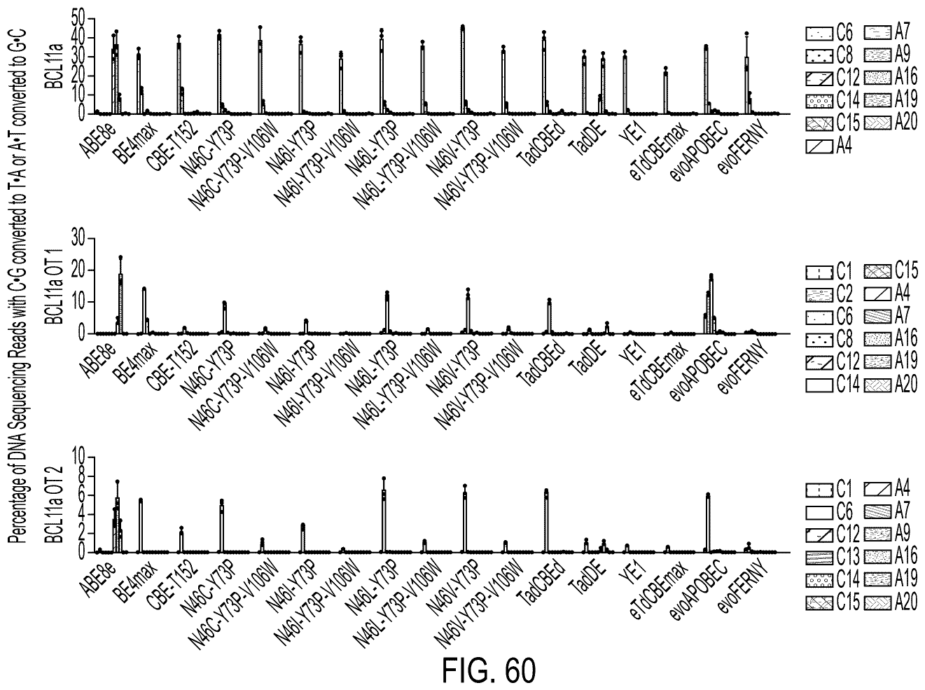

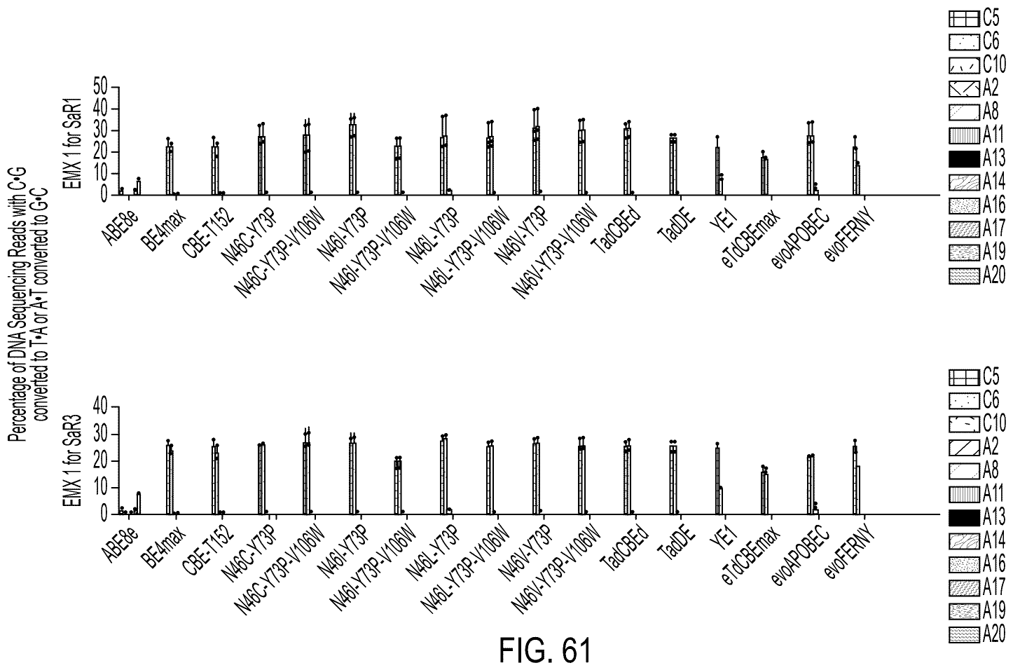

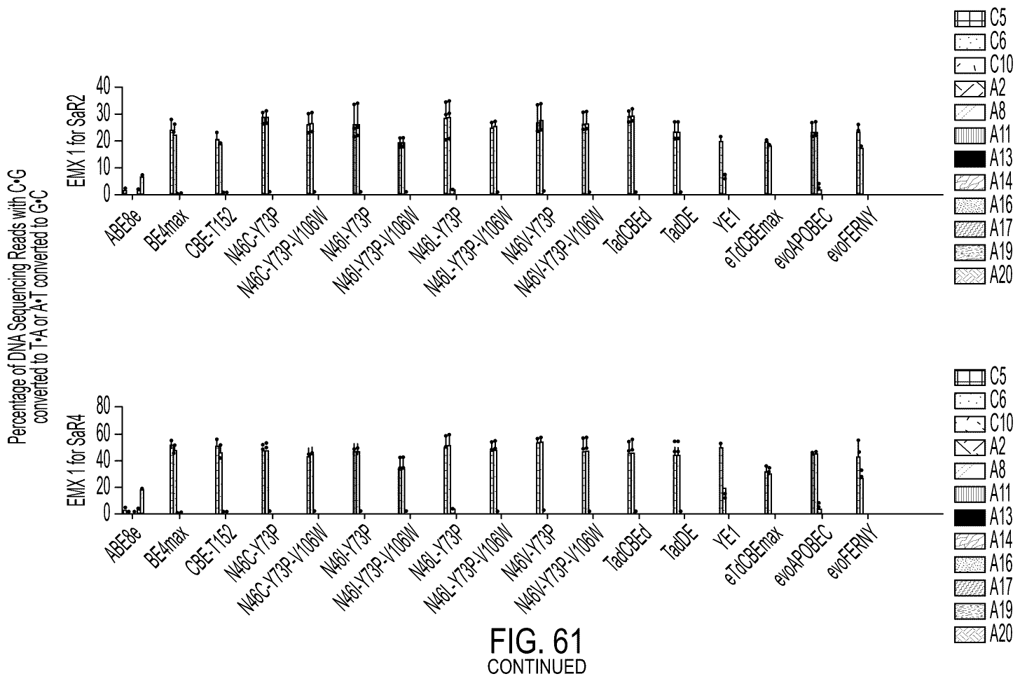

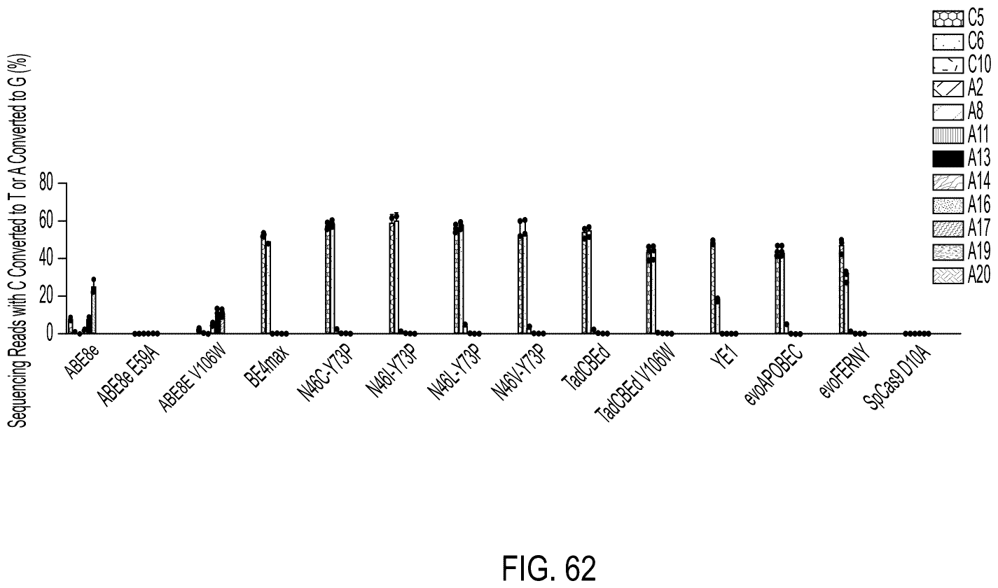

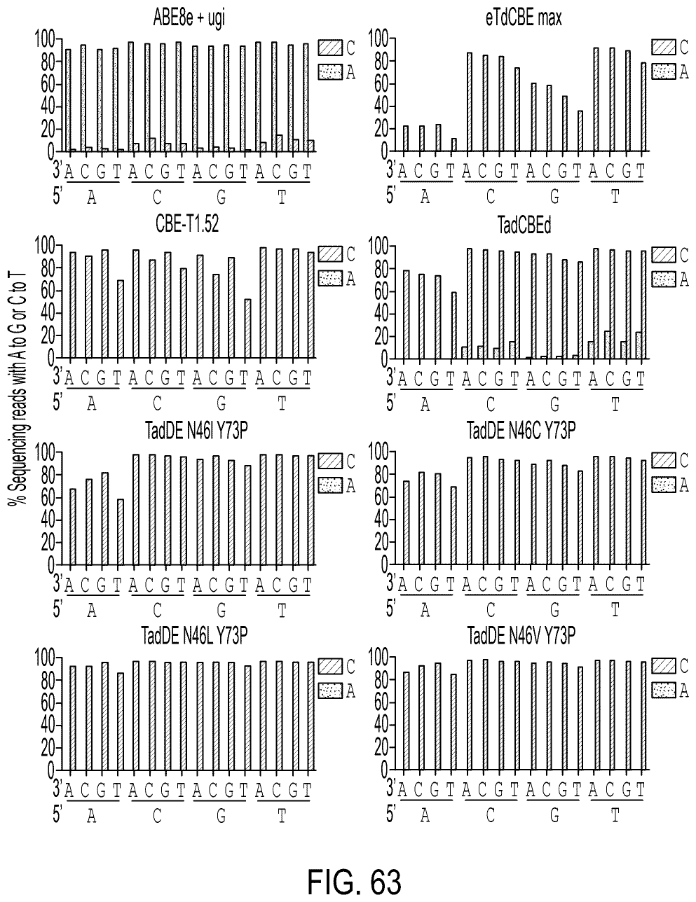

The results from this experiment are shown in FIGS. 54A and 54B.

### REFERENCES CITED

1. Eisenstein, M. Base editing marches on the clinic. Nat Biotechnol 40, 623-625 (2022).
2. ISRCTN—ISRCTN15323014: CAR T cells to fight T cell leukaemia. https://www.isrctn.com/ISRCTN15323014 doi:10.1186/ISRCTN15323014.
3. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
4. Gaudelli, N. M. et al. Programmable base editing of A·T to G·C in genomic DNA without DNA cleavage. Nature 551, 464-471 (2017).
5. Mok, B. Y. et al. A bacterial cytidine deaminase toxin enables CRISPR-free mitochondrial base editing. Nature 583, 631-637 (2020).
6. Mok, B. Y. et al. CRISPR-free base editors with enhanced activity and expanded targeting scope in mitochondrial and nuclear DNA. Nat Biotechnol (2022) doi:10.1038/s41587-022-01256-8.
7. Nishida, K. et al. Targeted nucleotide editing using hybrid prokaryotic and vertebrate adaptive immune systems. Science 353, aaf8729 (2016).
8. Gaudelli, N. M. et al. Directed evolution of adenine base editors with increased activity and therapeutic application. Nat Biotechnol 38, 892-900 (2020).
9. Richter, M. F. et al. Phage-assisted evolution of an adenine base editor with improved Cas domain compatibility and activity. Nat Biotechnol 38, 883-891 (2020).
10. Cho, S.-I. et al. Targeted A-to-G base editing in human mitochondrial DNA with programmable deaminases. Cell 185, 1764-1776.e12 (2022).
11. Arbab, M. et al. Determinants of Base Editing Outcomes from Target Library Analysis and Machine Learning. Cell 182, 463-480.e30 (2020).
12. Thuronyi, B. W. et al. Continuous evolution of base editors with expanded target compatibility and improved activity. Nat Biotechnol 37, 1070-1079 (2019).
13. Jin, S. et al. Cytosine, but not adenine, base editors induce genome-wide off-target mutations in rice. Science 364, 292-295 (2019).
14. Zuo, E. et al. Cytosine base editor generates substantial off-target single-nucleotide variants in mouse embryos. Science 364, 289-292 (2019).
15. Doman, J. L., Raguram, A., Newby, G. A. & Liu, D. R. Evaluation and minimization of Cas9-independent off-target DNA editing by cytosine base editors. Nat Biotechnol 38, 620-628 (2020).
16. Chester, A., Weinreb, V., Carter, C. W. & Navaratnam, N. Optimization of apolipoprotein B mRNA editing by APOBEC1 apoenzyme and the role of its auxiliary factor, ACF. RNA 10, 1399-1411 (2004).
17. Kim, J. et al. Structural and kinetic characterization of Escherichia coli TadA, the wobble-specific tRNA deaminase. Biochemistry 45, 6407-6416 (2006).
18. Lapinaite, A. et al. DNA capture by a CRISPR-Cas9-guided adenine base editor. Science 369, 566-571 (2020).
19. Yu, Y. et al. Cytosine base editors with minimized unguided DNA and RNA off-target events and high on-target activity. Nat Commun 11, 2052 (2020).
20. Rees, H. A. et al. Improving the DNA specificity and applicability of base editing through protein engineering and protein delivery. Nat Commun 8, 15790 (2017).
21. Grünewald, J. et al. Transcriptome-wide off-target RNA editing induced by CRISPR-guided DNA base editors. Nature 569, 433-437 (2019).
22. Kim, Y. B. et al. Increasing the genome-targeting scope and precision of base editing with engineered Cas9-cytidine deaminase fusions. Nat Biotechnol 35, 371-376 (2017).
23. Gehrke, J. M. et al. An APOBEC3A-Cas9 base editor with minimized bystander and off-target activities. Nat Biotechnol 36, 977-982 (2018).
24. Berríos, K. N. et al. Controllable genome editing with split-engineered base editors. Nat Chem Biol 17, 1262-1270 (2021).
25. Qiao, Q. et al. AID Recognizes Structured DNA for Class Switch Recombination. Mol Cell 67, 361-373.e4 (2017).
26. Wang, X. et al. Efficient base editing in methylated regions with a human APOBEC3A-Cas9 fusion. Nat Biotechnol 36, 946-949 (2018).
27. Davis, J. R. et al. Efficient in vivo base editing via single adeno-associated viruses with size-optimized genomes encoding compact adenine base editors. Nat Biomed Eng (2022) doi:10.1038/s41551-022-00911-4.
28. Zhang, H. et al. Adenine Base Editing in vivo with a Single Adeno-Associated Virus Vector. bioRxiv 2021.12.13.472434 Preprint at https://doi.org/10.1101/2021.12.13.472434 (2022).
29. Iyer, L. M., Zhang, D., Rogozin, I. B. & Aravind, L. Evolution of the deaminase fold and multiple origins of eukaryotic editing and mutagenic nucleic acid deaminases from bacterial toxin systems. Nucleic Acids Res 39, 9473-9497 (2011).
30. Rubio, M. A. T. et al. An adenosine-to-inosine tRNA-editing enzyme that can perform C-to-U deamination of DNA. Proc Natl Acad Sci USA 104, 7821-7826 (2007).
31. Kim, H. S., Jeong, Y. K., Hur, J. K., Kim, J.-S. & Bae, S. Adenine base editors catalyze cytosine conversions in human cells. Nat Biotechnol 37, 1145-1148 (2019).
32. Jeong, Y. K. et al. Adenine base editor engineering reduces editing of bystander cytosines. Nat Biotechnol 39, 1426-1433 (2021).
33. Abudayyeh, O. O. et al. A cytosine deaminase for programmable single-base RNA editing. Science 365, 382-386 (2019).
34. Rees, H. A., Wilson, C., Doman, J. L. & Liu, D. R. Analysis and minimization of cellular RNA editing by DNA adenine base editors. Sci Adv 5, eaax5717 (2019).
35. Badran, A. H. et al. Continuous evolution of Bacillus thuringiensis toxins overcomes insect resistance. Nature 533, 58-63 (2016).
36. Bryson, D. I. et al. Continuous directed evolution of aminoacyl-tRNA synthetases. Nat Chem Biol 13, 1253-1260 (2017).
37. Hubbard, B. P. et al. Continuous directed evolution of DNA-binding proteins to improve TALEN specificity. Nat Methods 12, 939-942 (2015).
38. Miller, S. M. et al. Continuous evolution of SpCas9 variants compatible with non-G PAMs. Nat Biotechnol 38, 471-481 (2020).
39. Brödel, A. K., Rodrigues, R., Jaramillo, A. & Isalan, M. Accelerated evolution of a minimal 63-amino acid dual transcription factor. Sci Adv 6, eaba2728 (2020).
40. Dickinson, B. C., Packer, M. S., Badran, A. H. & Liu, D. R. A system for the continuous directed evolution of proteases rapidly reveals drug-resistance mutations. Nat Commun 5, 5352 (2014).
41. Blum, T. R. et al. Phage-assisted evolution of botulinum neurotoxin proteases with reprogrammed specificity. Science 371, 803-810 (2021).
42. Pu, J., Zinkus-Boltz, J. & Dickinson, B. C. Evolution of a split RNA polymerase as a versatile biosensor platform. Nat Chem Biol 13, 432-438 (2017).
43. Roth, T. B., Woolston, B. M., Stephanopoulos, G. & Liu, D. R. Phage-Assisted Evolution of Bacillus methanolicus Methanol Dehydrogenase 2. ACS Synth Biol 8, 796-806 (2019).
44. Jones, K. A., Snodgrass, H. M., Belsare, K., Dickinson, B. C. & Lewis, J. C. Phage-Assisted Continuous Evolution and Selection of Enzymes for Chemical Synthesis. ACS Cent. Sci. 7, 1581-1590 (2021).
45. Johnston, C. W., Badran, A. H. & Collins, J. J. Continuous bioactivity-dependent evolution of an antibiotic biosynthetic pathway. Nat Commun 11, 4202 (2020).
46. Esvelt, K. M., Carlson, J. C. & Liu, D. R. A system for the continuous directed evolution of biomolecules. Nature 472, 499-503 (2011).
47. Badran, A. H. & Liu, D. R. Development of potent in vivo mutagenesis plasmids with broad mutational spectra. Nat Commun 6, 8425 (2015).
48. Koblan, L. W. et al. Improving cytidine and adenine base editors by expression optimization and ancestral reconstruction. Nat Biotechnol 36, 843-846 (2018).
49. Sakata, R. C. et al. Base editors for simultaneous introduction of C-to-T and A-to-G mutations. Nat Biotechnol 38, 865-869 (2020).
50. Xie, J. et al. ACBE, a new base editor for simultaneous C-to-T and A-to-G substitutions in mammalian systems. BMC Biol 18, 131 (2020).
51. Zhang, X. et al. Dual base editor catalyzes both cytosine and adenine base conversions in human cells. Nat Biotechnol 38, 856-860 (2020).
52. Grünewald, J. et al. A dual-deaminase CRISPR base editor enables concurrent adenine and cytosine editing. Nat Biotechnol 38, 861-864 (2020).
53. Liang, Y. et al. AGBE: a dual deaminase-mediated base editor by fusing CGBE with ABE for creating a saturated mutant population with multiple editing patterns. Nucleic Acids Res 50, 5384-5399 (2022).
54. Li, C. et al. Targeted, random mutagenesis of plant genes with dual cytosine and adenine base editors. Nat Biotechnol 38, 875-882 (2020).
55. Hanna, R. E. et al. Massively parallel assessment of human variants with base editor screens. Cell 184, 1064-1080.e20 (2021).
56. Cuella-Martin, R. et al. Functional interrogation of DNA damage response variants with base editing screens. Cell 184, 1081-1097.e19 (2021).
57. Jumper, J. et al. Highly accurate protein structure prediction with AlphaFold. Nature 596, 583-589 (2021).
58. Grünewald, J. et al. CRISPR DNA base editors with reduced RNA off-target and self-editing activities. Nat Biotechnol 37, 1041-1048 (2019).
59. Zhou, C. et al. Off-target RNA mutation induced by DNA base editing and its elimination by mutagenesis. Nature 571, 275-278 (2019).
60. Park, S. & Beal, P. A. Off-Target Editing by CRISPR-Guided DNA Base Editors. Biochemistry 58, 3727-3734 (2019).
61. Kleinstiver, B. P. et al. High-fidelity CRISPR-Cas9 nucleases with no detectable genome-wide off-target effects. Nature 529, 490-495 (2016).
62. Edraki, A. et al. A Compact, High-Accuracy Cas9 with a Dinucleotide PAM for In Vivo Genome Editing. Mol Cell 73, 714-726.e4 (2019).
63. Huang, T. P. et al. High-throughput continuous evolution of compact Cas9 variants targeting single-nucleotide-pyrimidine PAMs. Nature Biotechnology Under revision.
64. Ran, F. A. et al. In vivo genome editing using Staphylococcus aureus Cas9. Nature 520, 186-191 (2015).
65. Chelico, L., Pham, P., Calabrese, P. & Goodman, M. F. APOBEC3G DNA deaminase acts processively 3′-->5′ on single-stranded DNA. Nat Struct Mol Biol 13, 392-399 (2006).
66. Anzalone, A. V., Koblan, L. W. & Liu, D. R. Genome editing with CRISPR-Cas nucleases, base editors, transposases and prime editors. Nat Biotechnol 38, 824-844 (2020).
67. Song, Y. et al. Large-Fragment Deletions Induced by Cas9 Cleavage while Not in the BEs System. Mol Ther Nucleic Acids 21, 523-526 (2020).
68. Kosicki, M., Tomberg, K. & Bradley, A. Repair of double-strand breaks induced by CRISPR-Cas9 leads to large deletions and complex rearrangements. Nat Biotechnol 36, 765-771 (2018).
69. Ihry, R. J. et al. p53 inhibits CRISPR-Cas9 engineering in human pluripotent stem cells. Nat Med 24, 939-946 (2018).
70. Alanis-Lobato, G. et al. Frequent loss of heterozygosity in CRISPR-Cas9-edited early human embryos. Proc Natl Acad Sci USA 118, e2004832117 (2021).
71. Enache, O. M. et al. Cas9 activates the p53 pathway and selects for p53-inactivating mutations. Nat Genet 52, 662-668 (2020).
72. Knipping, F. et al. Disruption of HIV-1 co-receptors CCR5 and CXCR4 in primary human T cells and hematopoietic stem and progenitor cells using base editing. Mol Ther 30, 130-144 (2022).
73. Canver, M. C. et al. BCL11A enhancer dissection by Cas9-mediated in situ saturating mutagenesis. Nature 527, 192-197 (2015).
74. Wu, Y. et al. Highly efficient therapeutic gene editing of human hematopoietic stem cells. Nat Med 25, 776-783 (2019).
75. Carlson, J. C., Badran, A. H., Guggiana-Nilo, D. A. & Liu, D. R. Negative selection and stringency modulation in phage-assisted continuous evolution. Nat Chem Biol 10, 216-222 (2014).
76. Miller, S. M., Wang, T. & Liu, D. R. Phage-assisted continuous and non-continuous evolution. Nat Protoc 15, 4101-4127 (2020).
77. Clement, K. et al. CRISPResso2 provides accurate and rapid genome editing sequence analysis. Nat Biotechnol 37, 224-226 (2019).
78. Chen, L., Zhu, B., Ru, G. et al. Re-engineering the adenine deaminase TadA-8e for efficient and specific CRISPR-based cytosine base editing. Nat Biotechnol 41, 663-672 (2023). https://doi.org/10.1038/s41587-022-01532-7
79. Lam, D. K., Feliciano, P. R., Arif, A. et al. Improved cytosine base editors generated from TadA variants. Nat Biotechnol 41, 686-697 (2023). https://doi.org/10.1038/s41587-022-01611-9
80. Chadwick, A. C., Wang, X., & Musunuru, K. In Vivo Base Editing of PCSK9 (Proprotein Convertase Subtilisin/Kexin Type 9) as a Therapeutic Alternative to Genome Editing. Arteriosclerosis, thrombosis, and vascular biology, 37(9), 1741-1747 (2017). https://doi.org/10.1161/ATVBAHA.117.309881

### Supplemental Information for Examples 1-7

TABLE 1
Plasmids and selection phage (SP) described herein.

| Name | Usage (resistance) | Origin | ORF1 | ORF2 |
|---|---|---|---|---|
| pBT44c | Circuits 1 and 2 (KmR) | p15a | proC.SD8.intC.dCas9.UGI | |
| pMN404 | Circuit 1 (CbR) | SC101- | proT7.sd8.gIII.luxAB | proLac.sgRNA |
| | | E93K | | [Circuit 1] |
| pMN405 | Circuit 1 (SpR) | ColE1 | proA.SynRBS 0.4k.T7- | |
| | | | RNAP-degron | |
| pMN406 | Circuit 1 (SpR) | ColE1 | proB.SynRBS 0.4k.T7- | |
| | | | RNAP-degron | |
| pMN407 | Circuit 1 (SpR) | ColE1 | proC.SynRBS 0.4k.T7- | |
| | | | RNAP-degron | |
| pMN408 | Circuit 1 (SpR) | ColE1 | proD.SynRBS 0.4k.T7- | |
| | | | RNAP-degron | |
| pBT120b | Circuit 2 (CbR) | SC101- | proT7.sd8.gIII.luxAB | proLac.sgRNA |
| | | E93K | | [Circuit 2] |
| pBT2dR3- | Circuit 2 (SpR) | ColE1 | proA.SynRBS 0.4k.T7- | |
| ProA | | | RNAP-degron | |
| pBT2dR3- | Circuit 2 (SpR) | ColE1 | proB.SynRBS 0.4k.T7- | |
| ProB | | | RNAP-degron | |
| pBT2dR3- | Circuit 2 (SpR) | ColE1 | proC.SynRBS 0.4k.T7- | |
| ProC | | | RNAP-degron | |
| pBT2dR3- | Circuit 2 (SpR) | ColE1 | proD.SynRBS 0.4k.T7- | |
| ProD | | | RNAP-degron | |
| pMN618 | E. coli editing (CbR) | SC101 | pBAD.SD8.EvoAPOBEC1- | proLac.sgRNA |
| | | | 8e.dSpCas9-UGI-UGI | [Circuit 2] |
| pMN619 | E. coli editing (CbR) | SC101 | pBAD.SD8.TadA- | proLac.sgRNA |
| | | | 8e.dSpCas9-UGI-UGI | [Circuit 2] |
| pMN620 | E. coli editing (CbR) | SC101 | pBAD.SD8.TadA- | proLac.sgRNA |
| | | | CDa.dSpCas9-UGI-UGI | [Circuit 2] |
| pMN621 | E. coli editing (CbR) | SC101 | pBAD.SD8.TadA- | proLac.sgRNA |
| | | | CDb.dSpCas9-UGI-UGI | [Circuit 2] |
| pMN622 | E. coli editing (CbR) | SC101 | pBAD.SD8.TadA- | proLac.sgRNA |
| | | | CDc.dSpCas9-UGI-UGI | [Circuit 2] |
| pMN415 | E. coli editing target | RSF1030 | Target: | |
| | (KmR) | | ACTTCGCGTTCGCCTGG | |
| | | | ACC, PAM = AGG | |
| pMN573 | BE4max IVT template | pUC | proT7 (mutated).BE4max | |
| pMN574 | EvoFERNY IVT | pUC | proT7 (mutated).EvoFERNY | |
| | template | | | |
| pMN575 | EvoAPOBEC IVT | pUC | proT7 (mutated).EvoA | |
| | template | | | |
| pMN576 | TadCBEa IVT template | pUC | proT7 (mutated).TadCBEa | |
| pMN577 | TadCBEb IVT template | pUC | proT7 (mutated).TadCBEb | |
| pMN578 | TadCBEc IVT template | pUC | proT7 (mutated).TadCBEc | |
| pMN579 | TadCBEd IVT template | pUC | proT7 (mutated).TadCBEd | |
| pMN580 | TadCBEe IVT template | pUC | proT7 (mutated).TadCBEe | |
| pMN582 | YE1 IVT template | PUC | proT7 (mutated).YE1 | |
| pMN731 | TadCBEa V106W IVT | pUC | proT7 (mutated).TadCBEa | |
| | template | | | |
| pMN732 | TadCBEb V106W IVT | pUC | proT7 (mutated).TadCBEb | |
| | template | | | |
| pMN733 | TadCBEc V106W IVT | pUC | proT7 (mutated).TadCBEc | |
| | template | | | |
| pMN734 | TadCBEd V106W IVT | pUC | proT7 (mutated).TadCBEd | |
| | template | | | |
| pMN735 | TadCBEe V106W IVT | pUC | proT7 (mutated).TadCBEe | |
| | template | | | |
| pMN446 | Mammalian expression, | pUC | pCMV.TadCBEa | |
| | SpCas9 | | | |
| pMN447 | Mammalian expression, | pUC | pCMV.TadCBEb | |
| | SpCas9 | | | |
| pMN448 | Mammalian expression, | pUC | pCMV.TadCBEc | |
| | SpCas9 | | | |
| pMN450 | Mammalian expression, | pUC | pCMV.TadCBEd | |
| | SpCas9 | | | |
| pMN584 | Mammalian expression, | pUC | pCMV.TadCBEe | |
| | SpCas9 | | | |
| pMN554 | Mammalian expression, | pUC | pCMV.ABE8e V106W-UGI- | |
| | SpCas9 | | UGI | |
| pMN556 | Mammalian expression, | pUC | pCMV.TadCBEa V106W | |
| | SpCas9 | | | |
| pMN557 | Mammalian expression, | pUC | pCMV.TadCBEb V106W | |
| | SpCas9 | | | |
| pMN558 | Mammalian expression, | pUC | pCMV.TadCBEc V106W | |
| | SpCas9 | | | |
| pMN560 | Mammalian expression, | pUC | pCMV.TadCBEd V106W | |
| | SpCas9 | | | |
| pMN585 | Mammalian expression, | pUC | pCMV.TadCBEe V106W | |
| | SpCas9 | | | |
| pMN597 | Mammalian expression, | pUC | pCMV.TadDE | |
| | SpCas9 | | | |
| pMN599 | Mammalian expression, | pUC | pCMV.ABE8e-UGI-UGI | |
| | eNme2-C | | | |
| pMN607 | Mammalian expression, | pUC | pCMV.BE4max (eNme2-C) | |
| | eNme2-C | | | |
| pMN608 | Mammalian expression, | pUC | pCMV.EvoFERNY (eNme2-C) | |
| | eNme2-C | | | |
| pMN609 | Mammalian expression, | pUC | pCMV.EvoA (eNme2-C) | |
| | eNme2-C | | | |
| pMN601 | Mammalian expression, | pUC | pCMV.TadCBEa (eNme2-C) | |
| | eNme2-C | | | |
| pMN602 | Mammalian expression, | pUC | pCMV.TadCBEb (eNme2-C) | |
| | eNme2-C | | | |
| pMN603 | Mammalian expression, | pUC | pCMV.TadCBEc (eNme2-C) | |
| | eNme2-C | | | |
| pMN605 | Mammalian expression, | pUC | pCMV.TadCBEd (eNme2-C) | |
| | eNme2-C | | | |
| pMN606 | Mammalian expression, | pUC | pCMV.TadCBEe (eNme2-C) | |
| | eNme2-C | | | |
| pMN709 | Mammalian expression, | pUC | pCMV.TadDE (eNme2-C | |
| | eNme2-C S6P | | S6P) | |
| pMN745 | Mammalian expression, | pUC | pCMV.BE4max (SaCas9) | |
| | SaCas9 | | | |
| pMN746 | Mammalian expression, | pUC | pCMV.evoA (SaCas9) | |
| | SaCas9 | | | |
| pMN747 | Mammalian expression, | pUC | pCMV.evoFERNY (SaCas9) | |
| | SaCas9 | | | |
| pMN748 | Mammalian expression, | pUC | pCMV.ABE8e-UGI-UGI | |
| | SaCas9 | | (SaCas9) | |
| pMN749 | Mammalian expression, | pUC | pCMV.TadCBEa (SaCas9) | |
| | SaCas9 | | | |
| pMN750 | Mammalian expression, | pUC | pCMV.TadCBEb (SaCas9) | |
| | SaCas9 | | | |
| pMN751 | Mammalian expression, | pUC | pCMV.TadCBEc (SaCas9) | |
| | SaCas9 | | | |
| pMN752 | Mammalian expression, | pUC | pCMV.TadCBEd (SaCas9) | |
| | SaCas9 | | | |
| pMN753 | Mammalian expression, | pUC | pCMV.TadCBEe (SaCas9) | |
| | SaCas9 | | | |
| pMN754 | Mammalian expression, | pUC | pCMV.TadDE (SaCas9) | |
| | SaCas9 | | | |
| pMN589 | Mammalian expression, | pUC | pCMV.ABE8e E59A-UGI- | |
| | SpCas9 | | UGI | |
| pMN739 | Mammalian expression, | pUC | p2T-CMV.BE4max.BlastR | |
| | SpCas9 library analysis | | | |
| pMN717 | Mammalian expression, | pUC | p2T-CMV.TadCBEa.BlastR | |
| | SpCas9 library analysis | | | |
| pMN718 | Mammalian expression, | pUC | p2T-CMV.TadCBEb.BlastR | |
| | SpCas9 library analysis | | | |
| pMN719 | Mammalian expression, | pUC | p2T-CMV.TadCBEc.BlastR | |
| | SpCas9 library analysis | | | |
| pMN720 | Mammalian expression, | pUC | p2T-CMV.TadCBEd.BlastR | |
| | SpCas9 library analysis | | | |
| pMN721 | Mammalian expression, | pUC | p2T-CMV.TadCBEe.BlastR | |
| | SpCas9 library analysis | | | |
| pMN723 | Mammalian expression, | pUC | p2T-CMV.TadCBEa | |
| | SpCas9 library analysis | | V106W.BlastR | |
| pMN724 | Mammalian expression, | pUC | p2T-CMV.TadCBEb | |
| | SpCas9 library analysis | | V106W.BlastR | |
| pMN725 | Mammalian expression, | pUC | p2T-CMV.TadCBEc | |
| | SpCas9 library analysis | | V106W.BlastR | |
| pMN726 | Mammalian expression, | pUC | p2T-CMV.TadCBEd | |
| | SpCas9 library analysis | | V106W.BlastR | |
| SPMN21 | PA(N)CE, ΔgIII SP | M13f1 | p_gIII.SD4.TadA-8e-NpuN | |

Tables 2A-2E. Target protospacers and amplicons described herein with corresponding primers used for genomic DNA amplification.

TABLE 2A
SpCas9 genomic loci:

| Site | Proto- | | | | |
|---|---|---|---|---|---|
| Name | spacer | PAM | Amplicon | HTS-F | HTS-R |
| FANCF | GGAATCC | TGG | CATTGCAGAGAGGCGTAT | ACACTCTT | TGGAGT |
| | CTTCTGC | | CATTTCGCGGATGTTCCAA | TCCCTACA | TCAGAC |
| | AGCACC | | TCAGTACGCAGAGAGTCG | CGACGCTC | GTGTGC |
| | (SEQ ID | | CCGTCTCCAAGGTGAAAG | TTCCGATC | TCTTCC |
| | NO: 208) | | CGGAAGTAGGGCCTTCGC | TNNNNCA | GATCTG |
| | | | GCACCTCATGGAATCCCTT | TTGCAGA | GGGTCC |
| | | | CTGCAGCACCTGGATCGCT | GAGGCGT | CAGGTG |
| | | | TTTCCGAGCTTCTGGCGGT | ATCA (SEQ | CTGAC |
| | | | CTCAAGCACTACCTACGTC | ID NO: 32) | (SEQ ID |
| | | | AGCACCTGGGACCCC (SEQ | | NO: 66) |
| | | | ID NO: 7) | | |
| EMX1 | GAGTCCG | GGG | CAGCTCAGCCTGAGTGTTG | ACACTCTT | TGGAGT |
| | AGCAGAA | | AGGCCCCAGTGGCTGCTCT | TCCCTACA | TCAGAC |
| | GAAGAA | | GGGGGCCTCCTGAGTTTCT | CGACGCTC | GTGTGC |
| | (SEQ ID | | CATCTGTGCCCCTCCCTCC | TTCCGATC | TCTTCC |
| | NO: 209) | | CTGGCCCAGGTGAAGGTG | TNNNNCA | GATCTC |
| | | | TGGTTCCAGAACCGGAGG | GCTCAGCC | TCGTGG |
| | | | ACAAAGTACAAACGGCAG | TGAGTGTT | GTTTGT |
| | | | AAGCTGGAGGAGGAAGGG | GA (SEQ ID | GGTTGC |
| | | | CCTGAGTCCGAGCAGAAG | NO: 55) | (SEQ ID |
| | | | AAGAAGGGCTCCCATCAC | | NO: 67) |
| | | | ATCAACCGGTGGCGCATT | | |
| | | | GCCACGAAGCAGGCCAAT | | |
| | | | GGGGAGGACATCGATGTC | | |
| | | | ACCTCCAATGACTAGGGT | | |
| | | | GGGCAACCACAAACCCAC | | |
| | | | GAG (SEQ ID NO: 8) | | |
| BCL11A | TTTATCA | GGG | GCCAGAAAAGAGATATGG | ACACTCTT | TGGAGT |
| | CAGGCTC | | CATCTACTCTTAGACATAA | TCCCTACA | TCAGAC |
| | CAGGAA | | CACACCAGGGTCAATACA | CGACGCTC | GTGTGC |
| | (SEQ ID | | ACTTTGAAGCTAGTCTAGT | TTCCGATC | TCTTCC |
| | NO: 210) | | GCAAGCTAACAGTTGCTTT | TNNNNGC | GATCTA |
| | | | TATCACAGGCTCCAGGAA | CAGAAAA | GAGAGC |
| | | | GGGTTTGGCCTCTGATTAG | GAGATAT | CTTCCG |
| | | | GGTGGGGGCGTGGGTGGG | GGCATC | AAAGAG |
| | | | GTAGAAGAGGACTGGCAG | (SEQ ID | G (SEQ ID |
| | | | ACCTCTCCATCGGTGGCCG | NO: 56) | NO: 68) |
| | | | TTTGCCCAGGGGGGCCTCT | | |
| | | | TTCGGAAGGCTCTCT (SEQ | | |
| | | | ID NO: 9) | | |
| HEK3 | GGCCCAG | TGG | ATGTGGGCTGCCTAGAAA | ACACTCTT | TGGAGT |
| | ACTGAGC | | GGCATGGATGAGAGAAGC | TCCCTACA | TCAGAC |
| | ACGTGA | | CTGGAGACAGGGATCCCA | CGACGCTC | GTGTGC |
| | (SEQ ID | | GGGAAACGCCCATGCAAT | TTCCGATC | TCTTCC |
| | NO: 211) | | TAGTCTATTTCTGCTGCAA | TNNNNAT | GATCTC |
| | | | GTAAGCATGCATTTGTAGG | GTGGGCT | CCAGCC |
| | | | CTTGATGCTTTTTTTCTGCT | GCCTAGA | AAACTT |
| | | | TCTCCAGCCCTGGCCTGGG | AAGG (SEQ | GTCAAC |
| | | | TCAATCCTTGGGGCCCAGA | ID NO: 57) | C (SEQ ID |
| | | | CTGAGCACGTGATGGCAG | | NO: 69) |
| | | | AGGAAAGGAAGCCCTGCT | | |
| | | | TCCTCCAGAGGGCGTCGC | | |
| | | | AGGACAGCTTTTCCTAGAC | | |
| | | | AGGGGCTAGTATGTGCAG | | |
| | | | CTCCTGCACCGGGATACTG | | |
| | | | GTTGACAAGTTTGGCTGGG | | |
| | | | (SEQ ID NO: 10) | | |
| B2M | ACTCACG | AGG | GGCGGGCATTCCTGAAGC | ACACTCTT | TGGAGT |
| | CTGGATA | | TGACAGCATTCGGGCCGA | TCCCTACA | TCAGAC |
| | GCCTCC | | GATGTCTCGCTCCGTGGCC | CGACGCTC | GTGTGC |
| | (SEQ ID | | TTAGCTGTGCTCGCGCTAC | TTCCGATC | TCTTCC |
| | NO: 212) | | TCTCTCTTTCTGGCCTGGA | TNNNNGG | GATCTA |
| | | | GGCTATCCAGCGTGAGTCT | CGGGCATT | CTTGGA |
| | | | CTCCTACCCTCCCGCTCTG | CCTGAAG | GAAGGG |
| | | | GTCCTTCCTCTCCCGCTCT | CTGA (SEQ | AAGTCA |
| | | | GCACCCTCTGTGGCCCTCG | ID NO: 58) | CGGA |
| | | | CTGTGCTCTCTCGCTCCGT | | (SEQ ID |
| | | | GACTTCCCTTCTCCAAGT | | NO: 70) |
| | | | (SEQ ID NO: 11) | | |
| RNF2 | GTCATCT | AGG | ACGTCTCATATGCCCCTTG | ACACTCTT | TGGAGT |
| | TAGTCAT | | GCAGTCATCTTAGTCATTA | TCCCTACA | TCAGAC |
| | TACCTG | | CCTGAGGTGTTCGTTGTAA | CGACGCTC | GTGTGC |
| | (SEQ ID | | CTCATATAAACTGAGTTCC | TTCCGATC | TCTTCC |
| | NO: 213) | | CATGTTTTGCTTAATGGTT | TNNNNAC | GATCTA |
| | | | GAGTTCCGTTTGTCTGCAC | GTCTCATA | CGTAGG |
| | | | AGCCTGAGACATTGCTGG | TGCCCCTT | AATTTT |
| | | | AAATAAAGAAGAGAGAAA | GG (SEQ ID | GGTGGG |
| | | | AACAATTTTAGTATTTGGA | NO: 59) | ACA |
| | | | AGGGAAGTGCTATGGTCT | | (SEQ ID |
| | | | GAATGTATGTGTCCCACCA | | NO: 71) |
| | | | AAATTCCTACGT (SEQ ID | | |
| | | | NO: 12) | | |
| HEK | GGCACTG | GGG | GAACCCAGGTAGCCAGAG | ACACTCTT | TGGAGT |
| 4 | CGGCTGG | | ACCCGCTGGTCTTCTTTCC | TCCCTACA | TCAGAC |
| | AGGTGG | | CCTCCCCTGCCCTCCCCTC | CGACGCTC | GTGTGC |
| | (SEQ ID | | CCTTCAAGATGGCTGACA | TTCCGATC | TCTTCC |
| | NO: 214) | | AAGGCCGGGCTGGGTGGA | TNNNNGA | GATCTT |
| | | | AGGAAGGGAGGAAGGGCG | ACCCAGG | CCTTTC |
| | | | AGGCAGAGGGTCCAAAGC | TAGCCAG | AACCCG |
| | | | AGGATGACAGGCAGGGGC | AGAC (SEQ | AACGGA |
| | | | ACCGCGGCGCCCCGGTGG | ID NO: 60) | G (SEQ ID |
| | | | CACTGCGGCTGGAGGTGG | | NO: 72) |
| | | | GGGTTAAAGCGGAGACTC | | |
| | | | TGGTGCTGTGTGACTACAG | | |
| | | | TGGGGGCCCTGCCCTCTCT | | |
| | | | GAGCCCCCGCCTCCAGGC | | |
| | | | CTGTGTGTGTGTCTCCGTT | | |
| | | | CGGGTTGAAAGGA (SEQ ID | | |
| | | | NO: 13) | | |
| PDCD1 | CACCTAC | TGG | GACCTGCCAGGGACTGAG | ACACTCTT | TGGAGT |
| | CTAAGAA | | GGTGGAAGGTCCCTCCAG | TCCCTACA | TCAGAC |
| | CCATCC | | ACCCCTGGCTCTGGGACAC | CGACGCTC | GTGTGC |
| | (SEQ ID | | CTGACCGCCGACCCCACCT | TTCCGATC | TCTTCC |
| | NO: 215) | | ACCTAAGAACCATCCTGG | TNNNNGA | GATCTG |
| | | | CCGCCAGCCCAGTTGTAGC | CCTGCCAG | AGAAGG |
| | | | ACCGCCCAGACGACTGGC | GGACTGA | CGGCAC |
| | | | CAGGGCGCCTGTGGGATC | GGG (SEQ | TCTGGT |
| | | | TGCATGCCTGGAGCAGCC | ID NO: 61) | G (SEQ ID |
| | | | CCACCAGAGTGCCGCCTTC | | NO: 73) |
| | | | TC (SEQ ID NO: 14) | | |
| TRAC | TTCGTAT | AGG | TCTGCCTTGGGGAAAACC | ACACTCTT | TGGAGT |
| | CTGTAAA | | GTGGGTGTGTCCTGCAGGC | TCCCTACA | TCAGAC |
| | ACCAAG | | CATGCAGGCCTGGGACAT | CGACGCTC | GTGTGC |
| | (SEQ ID | | GCAAGCCCATAACCGCTG | TTCCGATC | TCTTCC |
| | NO: 216) | | TGGCCTCTTGGTTTTACAG | TNNNNTCT | GATCTC |
| | | | ATACGAACCTAAACTTTCA | GCCTTGGG | GCAGCG |
| | | | AAACCTGTCAGTGATTGG | GAAAACC | TCATGA |
| | | | GTTCCGAATCCTCCTCCTG | GT (SEQ ID | GCAGAT |
| | | | AAAGTGGCCGGGTTTAAT | NO: 62) | T (SEQ ID |
| | | | CTGCTCATGACGCTGCG | | NO: 79) |
| | | | (SEQ ID NO: 15) | | |
| R5-7 | CAATGTG | GGG | GCTGGTCATCCTCATCCTG | ACACTCTT | TGGAGT |
| | TCAACTC | | ATAAACTGCAAAAGGCTG | TCCCTACA | TCAGAC |
| | TTGACA | | AAGAGCATGACTGACATC | CGACGCTC | GTGTGC |
| | (SEQ ID | | TACCTGCTCAACCTGGCCA | TTCCGATC | TCTTCC |
| | NO: 217) | | TCTCTGACCTGTTTTTCCTT | TNNNNGC | GATCTT |
| | | | CTTACTGTCCCCTTCTGGG | TGGTCATC | TCCTGG |
| | | | CTCACTATGCTGCCGCCCA | CTCATCCT | GAGAGA |
| | | | GTGGGACTTTGGAAATAC | G (SEQ ID | CGCAAA |
| | | | AATGTGTCAACTCTTGACA | NO: 63) | C (SEQ ID |
| | | | GGGCTCTATTTTATAGGCT | | NO: 80) |
| | | | TCTTCTCTGGAATCTTCTT | | |
| | | | CATCATCCTCCTGACAATC | | |
| | | | GATAGGTACCTGGCTGTCG | | |
| | | | TCCATGCTGTGTTTGCTTT | | |
| | | | AAAAGCCAGGACGGTCAC | | |
| | | | CTTTGGGGTGGTGACAAGT | | |
| | | | GTGATCACTTGGGTGGTGG | | |
| | | | CTGTGTTTGCGTCTCTCCC | | |
| | | | AGGAA (SEQ ID NO: 16) | | |
| X4-2 | CAGTTTC | TGG | AAGCCAGGATGAGGATGA | ACACTCTT | TGGAGT |
| | AGCACAT | | CTGTGGTCTTGAGGGCCTT | TCCCTACA | TCAGAC |
| | CATGGT | | GCGCTTCTGGTGGCCCTTG | CGACGCTC | GTGTGC |
| | (SEQ ID | | GAGTGTGACAGCTTGGAG | TTCCGATC | TCTTCC |
| | NO: 218) | | ATGATAATGCAATAGCAG | TNNNNAA | GATCTC |
| | | | GACAGGATGACAATACCA | GCCAGGA | CTGCCC |
| | | | GGCAGGATAAGGCCAACC | TGAGGAT | TCCTGC |
| | | | ATGATGTGCTGAAACTGG | GACTG | TGACTA |
| | | | AACACAACCACCCACAAG | (SEQ ID | T (SEQ ID |
| | | | TCATTGGGGTAGAAGCGG | NO: 64) | NO: 81) |
| | | | TCACAGATATATCTGTCAT | | |
| | | | CTGCCTCACTGACGTTGGC | | |
| | | | AAAGATGAAGTCGGGAAT | | |
| | | | AGTCAGCAGGAGGGCAGG | | |
| | | | (SEQ ID NO: 17) | | |
| Circuit | GGTCCAG | CGG | GCGCTCGACACCGATATC | ACACTCTT | TGGAGT |
| 2 | GCGAACG | | ATAAACATTCTGTTTGCCT | TCCCTACA | TCAGAC |
| | CGAAGT | | AAATATTTACGCGTGGCA | CGACGCTC | GTGTGC |
| | (SEQ ID | | ATTTTGATTGAGCCACCTT | TTCCGATC | TCTTCC |
| | NO: 219) | | CTGCCCTGGTCCAGGCGA | TNNNNGA | GATCTG |
| | | | ACGCGAAGTCGGATCCTC | AGTGTTCG | CGCTCG |
| | | | CGGAATTCGGGAGATTAT | AATATTGT | ACACCG |
| | | | CCACGCGCATCAGGTCTA | CTGGAAG | ATATC |
| | | | ATTCGCGTTCAAAAATCTC | A (SEQ ID | (SEQ ID |
| | | | ATCAATGGGGAGCATCTG | NO: 65) | NO: 82) |
| | | | ACCATCTACAGTCATAAAT | | |
| | | | TTGTGATCCTTTGTGGCAC | | |
| | | | GGATTAAAGATCCGTCTTC | | |
| | | | CAGACAATATTCGAACAC | | |
| | | | TTC (SEQ ID NO: 18) | | |

TABLE 2B
eNme2-C genomic loci:

| Site | Proto- | | | | |
|---|---|---|---|---|---|
| Name | spacer | PAM | Amplicon | HTS-F | HTS-R |
| TS90 | GCAAGAG | AG | GCTCAGAAAAAGGGCCCTG | ACACTCTT | TGGAGT |
| | CACAAGA | AC | ACAACTCTTTTCATCTTCTA | TCCCTACA | TCAGAC |
| | GGAAGAG | CC | GGTATGACAACGAATTTGGC | CGACGCTC | GTGTGC |
| | AG (SEQ | | TACAGCAACAGGGTGGTGG | TTCCGATC | TCTTCC |
| | ID NO: | | ACCTCATGGCCCACATGGCC | TNNNNGCT | GATCTG |
| | 226) | | TCCAAGGAGTAAGACCCCT | CAGAAAA | AGATTC |
| | | | GGACCACCAGCCCCAGCAA | AGGGCCCT | AGTGTG |
| | | | GAGCACAAGAGGAAGAGAG | GA (SEQ ID | GTGGGG |
| | | | AGACCCTCACTGCTGGGGA | NO: 87) | G (SEQ |
| | | | GTCCCTGCCACACTCAGTCC | | ID NO: |
| | | | CCCACCACACTGAATCTC | | 91) |
| | | | (SEQ ID NO: 83) | | |
| eNme | GGCTACA | AC | GCTCAGAAAAAGGGCCCTG | ACACTCTT | TGGAGT |
| 2-C | GCAACAG | CT | ACAACTCTTTTCATCTTCTA | TCCCTACA | TCAGAC |
| site 5 | GGTGGTG | CA | GGTATGACAACGAATTTGGC | CGACGCTC | GTGTGC |
| | G (SEQ ID | | TACAGCAACAGGGTGGTGG | TTCCGATC | TCTTCC |
| | NO: 227) | | ACCTCATGGCCCACATGGCC | TNNNNGCT | GATCTG |
| | | | TCCAAGGAGTAAGACCCCT | CAGAAAA | AGATTC |
| | | | GGACCACCAGCCCCAGCAA | AGGGCCCT | AGTGTG |
| | | | GAGCACAAGAGGAAGAGAG | GA (SEQ ID | GTGGGG |
| | | | AGACCCTCACTGCTGGGGA | NO: 87) | G (SEQ |
| | | | GTCCCTGCCACACTCAGTCC | | ID NO: |
| | | | CCCACCACACTGAATCTC | | 91) |
| | | | (SEQ ID NO: 83) | | |
| eNme | GGTGTGC | GG | CATTCCCTCTTTAGCCAGAG | ACACTCTT | TGGAGT |
| 2-C | AGACGGC | GG | CCGGGGTGTGCAGACGGCA | TCCCTACA | TCAGAC |
| site | AGTCACT | CG | GTCACTAGGGGGCGCTCGG | CGACGCTC | GTGTGC |
| 66 | AG (SEQ | | CCACCACAGGGAAGCTGGG | TTCCGATC | TCTTCC |
| | ID NO: | | TGAATGGAGCGAGCAGCGT | TNNNNCAT | GATCTC |
| | 228) | | CTTCGAGAGTGAGGACGTGT | TCCCTCTT | AGATCT |
| | | | GTGTCTGTGTGGGTGAGTGA | TAGCCAG | ATTGGA |
| | | | GTGTGTGCGTGTGGGGTTGA | AGCCGG | ATCCTG |
| | | | GGGCGTTGGAGCGGGGAGA | (SEQ ID NO: | GAGTGA |
| | | | AGGCCAGGGGTCACTCCAG | 88) | CC (SEQ |
| | | | GATTCCAATAGATCTG (SEQ | | ID NO: |
| | | | ID NO: 84) | | 92) |
| eNme | GTCTCCG | CA | GGCGAGGCAGAGGGTCCAA | ACACTCTT | TGGAGT |
| 2-C | CTTTAAC | GC | AGCAGGATGACAGGCAGGG | TCCCTACA | TCAGAC |
| site | CCCCACC | CG | GCACCGCGGCGCCCCGGTG | CGACGCTC | GTGTGC |
| 50 | TC (SEQ ID | | GCACTGCGGCTGGAGGTGG | TTCCGATC | TCTTCC |
| | NO: 229) | | GGGTTAAAGCGGAGACTCT | TNNNNGG | GATCTC |
| | | | GGTGCTGTGTGACTACAGTG | CGAGGCA | TCCTTCT |
| | | | GGGGCCCTGCCCTCTCTGAG | GAGGGTC | GGGGCC |
| | | | CCCCCGCCTCCAGGCCTGTG | CAAA (SEQ | TTTTTCC |
| | | | TGTGTGTCTCCGTTCGGGTT | ID NO: 89) | C (SEQ ID |
| | | | GAAAGGAGCCCGGGAAAAA | | NO: 93) |
| | | | GGCCCCAGAAGGAG (SEQ ID | | |
| | | | NO: 85) | | |
| eNme | CCTCTTG | GC | CTACCTGCGCCACATCCATC | ACACTCTT | TGGAGT |
| 2-C | CCTCCAC | GG | GGCGCTTTGGTCGGCATGGC | TCCCTACA | TCAGAC |
| site | TGGTTGT | CT | CCCATTCGCACGGCTCTGGA | CGACGCTC | GTGTGC |
| 39 | GC (SEQ | | GCGGCGGCTGCACAACCAG | TTCCGATC | TCTTCC |
| | ID NO: | | TGGAGGCAAGAGGGCGGCT | TNNNNAA | GATCTC |
| | 230) | | TTGGGCGGGGTCCAGTTCCG | AGAGTTGC | TACCTG |
| | | | GGATTAGCGAACTTCCAGGC | TGCACCAG | CGCCAC |
| | | | CCTCGGTCACTGTGACGTCC | GT (SEQ ID | ATCCAT |
| | | | TGCTCTCTCTGCGCCTGCTG | NO: 90) | C (SEQ ID |
| | | | GAGAACCGGGCCCTCGGGG | | NO: 94) |
| | | | ATGCAGCTCGTTACCACCTG | | |
| | | | GTGCAGCAACTCTTT (SEQ | | |
| | | | ID NO: 86) | | |
| eNme | GGTCCAA | GG | GAACCCAGGTAGCCAGAGA | ACACTCTT | TGGAGT |
| 2-C | AGCAGGA | GG | CCCGCTGGTCTTCTTTCCCCT | TCCCTACA | TCAGAC |
| site | TGACAGG | CA | CCCCTGCCCTCCCCTCCCTT | CGACGCTC | GTGTGC |
| 11 | CA (SEQ | | CAAGATGGCTGACAAAGGC | TTCCGATC | TCTTCC |
| | ID NO: | | CGGGCTGGGTGGAAGGAAG | TNNNNGA | GATCTT |
| | 231) | | GGAGGAAGGGCGAGGCAGA | ACCCAGGT | CCTTTC |
| | | | GGGTCCAAAGCAGGATGAC | AGCCAGA | AACCCG |
| | | | AGGCAGGGGCACCGCGGCG | GAC (SEQ | AACGGA |
| | | | CCCCGGTGGCACTGCGGCTG | ID NO: 60) | G (SEQ |
| | | | GAGGTGGGGGTTAAAGCGG | | ID NO: |
| | | | AGACTCTGGTGCTGTGTGAC | | 76) |
| | | | TACAGTGGGGGCCCTGCCCT | | |
| | | | CTCTGAGCCCCCGCCTCCAG | | |
| | | | GCCTGTGTGTGTGTCTCCGT | | |
| | | | TCGGGTTGAAAGGA (SEQ ID | | |
| | | | NO: 13) | | |

TABLE 2C
SpCas9 Cas-dependent off-target sites:

| Site | Proto- | | | | |
|---|---|---|---|---|---|
| Name | spacer | PAM | Amplicon | HTS-F | HTS-R |
| HEK | CACCCAG | TG | TCCCCTGTTGACCTGGAGAA | ACACTCTT | TGGAGT |
| 3OT1 | ACTGAGC | G | GCATGAACCAGTCAAAAAG | TCCCTACA | TCAGAC |
| | ACGTGC | | TTTAAAGACAAGAGCATTA | CGACGCT | GTGTGC |
| | (SEQ ID | | ACTGCACCAGTGGGCAGCTC | CTTCCGAT | TCTTCCG |
| | NO: 232) | | AGCTCAGACACCAGTAGCG | CTNNNNT | ATCTCA |
| | | | TGGGCACCCAGACTGAGCA | CCCCTGTT | CTGTAC |
| | | | CGTGCTGGAGCCCAAGAAA | GACCTGG | TTGCCCT |
| | | | TGCAGAGACCTGTGCACCTC | AGAA (SEQ | GACCA |
| | | | TGGTCAGGGCAAGTACAGT | ID NO: 126) | (SEQ ID |
| | | | G (SEQ ID NO: 95) | | NO: 181) |
| HEK | GACACAG | GG | TTGGTGTTGACAGGGAGCA | ACACTCTT | TGGAGT |
| 3OT2 | ACTGGGC | G | ACTTCACAGTCCCAGGCATC | TCCCTACA | TCAGAC |
| | ACGTGA | | AGGACACAGACTGGGCACG | CGACGCT | GTGTGC |
| | (SEQ ID | | TGAGGGAAGCCCAAGGGAG | CTTCCGAT | TCTTCCG |
| | NO: 233) | | AGGACTGGTGTAATCGAGG | CTNNNNTT | ATCTCT |
| | | | CTGACTCCACTTTTAATGTT | GGTGTTG | GAGATG |
| | | | TGACTGATGATAGGTTTCAA | ACAGGGA | TGGGCA |
| | | | GTCTCACTAAGTCTCCTTCC | GCAA (SEQ | GAAGGG |
| | | | CCTTCTGCCCACATCTCAG | ID NO: 127) | (SEQ ID |
| | | | (SEQ ID NO: 96) | | NO: 182) |
| HEK | AGCTCAG | GG | TGAGAGGGAACAGAAGGGC | ACACTCTT | TGGAGT |
| 3OT3 | ACTGAGC | G | TAAGACTAAAAGGAACAGA | TCCCTACA | TCAGAC |
| | AAGTGA | | GGAGTTCATAGTGAGCGGT | CGACGCT | GTGTGC |
| | (SEQ ID | | AAAGAGCTCAGACTGAGCA | CTTCCGAT | TCTTCCG |
| | NO: 234) | | AGTGAGGGGCTCAGCCTCCC | CTNNNNT | ATCTGT |
| | | | ATGGAGGACAGGGGGCTGG | GAGAGGG | CCAAAG |
| | | | GGCCCCTGGCTGATGTCTGG | AACAGAA | GCCCAA |
| | | | ACTGAAGCCCCCACGCCCA | GGGCT | GAACCT |
| | | | GAGGTTCTTGGGCCTTTGGA | (SEQ ID | (SEQ ID |
| | | | C (SEQ ID NO: 97) | NO: 128) | NO: 183) |
| HEK | AGACCAG | GG | CCTAGCACTTTGGAAGGTCG | ACACTCTT | TGGAGT |
| 3OT4 | ACTGAGC | G | AAGCGGCAGGATGGCTTCA | TCCCTACA | TCAGAC |
| | AAGAGA | | ACCCAGGAGTTCGAGACCA | CGACGCT | GTGTGC |
| | (SEQ ID | | GACTGAGCAAGAGAGGGAG | CTTCCGAT | TCTTCCG |
| | NO: 235) | | AGTGTCTGTATTAACAACAA | CTNNNNT | ATCTGC |
| | | | ACAAACAAACAAAAAACTA | CCTAGCA | TCATCTT |
| | | | AACTAAAAGAAACTGTGGT | CTTTGGAA | AATCTG |
| | | | GTATAATATAAAATTCTGGC | GGTCG | CTCAGC |
| | | | TGAGCAGATTAAGATGAGC | (SEQ ID | C (SEQ ID |
| | | | (SEQ ID NO: 98) | NO: 129) | NO: 184) |
| HEK | GAGCCAG | GG | AAAGGAGCAGCTCTTCCTGG | ACACTCTT | TGGAGT |
| 3OT5 | AATGAGC | G | TGGAAATTGCGAGCAGAGG | TCCCTACA | TCAGAC |
| | ACGTGA | | CTGCGTGAGTTCCGTAACTC | CGACGCT | GTGTGC |
| | (SEQ ID | | GCACACAGCCTCCATTTGGA | CTTCCGAT | TCTTCCG |
| | NO: 236) | | GCCAGAATGAGCACGTGAG | CTNNNNA | ATCTGT |
| | | | GGACCCCGGGCAGAGGGGC | AAGGAGC | CTGCAC |
| | | | CAGTGCTGACATTATGCTCC | AGCTCTTC | CATCTC |
| | | | ATGCAACCTCCCATCCTGTT | CTGG (SEQ | CCACAA |
| | | | GTGGGAGATGGTGCAGAC | ID NO: 130) | (SEQ ID |
| | | | (SEQ ID NO: 99) | | NO: 185) |
| HEK | TGCACTG | TG | GGCATGGCTTCTGAGACTCA | ACACTCTT | TGGAGT |
| 4OT1 | CGGCCGG | G | TAGCTGGGGCTGAAGATCCC | TCCCTACA | TCAGAC |
| | AGGAGG | | TAGGGGGGCTCTGCTGGGCT | CGACGCT | GTGTGC |
| | (SEQ ID | | CACTGCTCTCCAGAGTGGTC | CTTCCGAT | TCTTCCG |
| | NO: 237) | | CAGCCCGGCTGCAGGGTGCT | CTNNNNG | ATCTGT |
| | | | GCTTCCAGCTTGGTGCACTG | GCATGGC | CTCCCTT |
| | | | CGGCCGGAGGAGGTGGAGG | TTCTGAGA | GCACTC |
| | | | ATGGAAAGTAAGATTCAAA | CTCA (SEQ | CCTGTCT |
| | | | GACAGGGAGTGCAAGGG | ID NO: 131) | TT (SEQ |
| | | | (SEQ ID NO: 105) | | ID NO: |
| | | | | | 186) |
| HEK | GGCTCTG | TG | TTTGGCAATGGAGGCATTGG | ACACTCTT | TGGAGT |
| 4OT2 | CGGCTGG | G | GCAGGGGAAGCCTGTCTTCA | TCCCTACA | TCAGAC |
| | AGGGGG | | GGGCACATGCACGTGCGCA | CGACGCT | GTGTGC |
| | (SEQ ID | | GGGCTCTGCGGCTGGAGGG | CTTCCGAT | TCTTCCG |
| | NO: 238) | | GGTGGGGTTGCTGTTAGTGA | CTNNNNTT | ATCTGA |
| | | | CAGGGGCCCCAGCCAGGCA | TGGCAAT | AGAGGC |
| | | | GGTTTCAGGATTGGGGAGC | GGAGGCA | TGCCCA |
| | | | ACTTGCTTCGGCTCCCTTGC | TTGG (SEQ | TGAGAG |
| | | | TCTCATGGGCAGCCTCTTC | ID NO: 132) | (SEQ ID |
| | | | (SEQ ID NO: 106) | | NO: 187) |
| HEK | GGCACGA | GG | GGTCTGAGGCTCGAATCCTG | ACACTCTT | TGGAGT |
| 4OT3 | CGGCTGG | G | GCAGCAGGTCCTTCATGGCA | TCCCTACA | TCAGAC |
| | AGGTGG | | AGGCGGGAAAAGAGAAAAG | CGACGCT | GTGTGC |
| | (SEQ ID | | CCAACGGGTTCTCATGCTGG | CTTCCGAT | TCTTCCG |
| | NO: 239) | | GAAAAGATGCCGGGCACGA | CTNNNNG | ATCTCT |
| | | | CGGCTGGAGGTGGGGGGTT | GTCTGAG | GTGGCC |
| | | | GGGAGTGGGTGGGATGCTT | GCTCGAA | TCCATA |
| | | | GCGTGCCCTGCATGAGGTGC | TCCTG | TCCCTG |
| | | | AGGGATATGGAGGCCACAG | (SEQ ID | (SEQ ID |
| | | | (SEQ ID NO: 107) | NO: 133) | NO: 188) |
| HEK | GGCATCA | AG | TTCCACCAGAACTCAGCCCA | ACACTCTT | TGGAGT |
| 4OT4 | CGGCTGG | G | GGCTGCTGTGGGATGGAATC | TCCCTACA | TCAGAC |
| | AGGTGG | | ACCTGCACCCGGATGTTCTT | CGACGCT | GTGTGC |
| | (SEQ ID | | TCTGGGCTGGTACATACAGG | CTTCCGAT | TCTTCCG |
| | NO: 240) | | CAAGGCATCACGGCTGGAG | CTNNNNTT | ATCTCCT |
| | | | GTGGAGGGGGCCTAACCCG | TCCACCA | CGGTTC |
| | | | GGGTTGCCCAGGAAGGGGT | GAACTCA | CTCCAC |
| | | | TTGCACATGGATTCGGTGTG | GCCC (SEQ | AACAC |
| | | | TTGTGGAGGAACCGAGG | ID NO: 134) | (SEQ ID |
| | | | (SEQ ID NO: 108) | | NO: 189) |
| HEK | GGCGCTG | AG | CACGGGAAGGACAGGAGAA | ACACTCTT | TGGAGT |
| 4OT5 | CGGCGGG | G | GGTGCTGGACCGCCTGGACT | TCCCTACA | TCAGAC |
| | AGGTGG | | TTGTGCTGACCAGCCTTGTG | CGACGCT | GTGTGC |
| | (SEQ ID | | GCGCTGCGGCGGGAGGTGG | CTTCCGAT | TCTTCCG |
| | NO: 241) | | AGGAGCTGAGAAGCAGCCT | CTNNNNC | ATCTGC |
| | | | GCGAGGGCTTGCGGGGGAG | ACGGGAA | AGGGGA |
| | | | ATTGTTGGGGAGGTCCGGTG | GGACAGG | GGGATA |
| | | | AGTAATGCGGCTTCTTCTCC | AGAAG | AAGCAG |
| | | | TGCTTTATCCCTCCCCTGC | (SEQ ID | (SEQ ID |
| | | | (SEQ ID NO: 109) | NO: 135) | NO: 190) |
| EMX1 | GAGTTAG | AG | TGCCCAATCATTGATGCTTT | ACACTCTT | TGGAGT |
| OT1 | AGCAGAA | G | TATACCATCTTGGGGTTACA | TCCCTACA | TCAGAC |
| | GAAGAA | | GAAAGAATAGGGGCTTATG | CGACGCT | GTGTGC |
| | (SEQ ID | | GCATGGCAAGACAGATTGT | CTTCCGAT | TCTTCCG |
| | NO: 242) | | CAGAGTTAGAGCAGAAGAA | CTNNNNT | ATCTAG |
| | | | GAAAGGCATGGAGTAAAGG | GCCCAAT | AAACAT |
| | | | CAATCTTGTGCAGATGTACA | CATTGATG | TTACCA |
| | | | GGTAGCAGCCCTCAGAAAA | CTTTT | TAGACT |
| | | | AATAGGTGATAGTCTATGGT | (SEQ ID | ATCACC |
| | | | AAATGTTTCT (SEQ ID NO: | NO: 136) | T (SEQ ID |
| | | | 110) | | NO: 191) |
| EMX1 | GAGTCTA | GA | GTAGCCTCTTTCTCAATGTG | ACACTCTT | TGGAGT |
| OT2 | AGCAGAA | G | CTTCAACCCATCACGGCCTT | TCCCTACA | TCAGAC |
| | GAAGAA | | TGCAAATAGAGCCCTTTATT | CGACGCT | GTGTGC |
| | (SEQ ID | | CATAGTAGACAAGAGTCTA | CTTCCGAT | TCTTCCG |
| | NO: 243) | | AGCAGAAGAAGAAGAGAGC | CTNNNNA | ATCTGC |
| | | | CACTACCCAACCATCTACTC | GTAGCCTC | TTTCAC |
| | | | TTCTAATGGTGTTTTCCTAC | TTTCTCAA | AAGGAT |
| | | | AAAGGCCAAGTCATGAGAC | TGTGC | GCAGTC |
| | | | TGCATCCTTGTGAAAGC | (SEQ ID | T (SEQ ID |
| | | | (SEQ ID NO: 111) | NO: 137) | NO: 192) |
| EMX1 | GAGGCCG | CG | GAGCTAGACTCCGAGGGGA | ACACTCTT | TGGAGT |
| OT3 | AGCAGAA | G | GGCTGCGAGCCGCAAGCGC | TCCCTACA | TCAGAC |
| | GAAAGA | | AGGAGCCGGGTGGGAGAGA | CGACGCT | GTGTGC |
| | (SEQ ID | | GACCCCTTCTTCTGCAAATG | CTTCCGAT | TCTTCCG |
| | NO: 244) | | AGGAGGCCGAGCAGAAGAA | CTNNNNG | ATCTTCC |
| | | | AGACGGCGACAGATGTTGG | AGCTAGA | TCGTCCT |
| | | | GGGGAGGGGACGGTTTGTG | CTCCGAG | GCTCTC |
| | | | AGGGATAGGGAGAGAAAGT | GGGA (SEQ | ACTT |
| | | | CTAAGTGAGAGCAGGACGA | ID NO: 138) | (SEQ ID |
| | | | GGA (SEQ ID NO: 112) | | NO: 193) |
| EMX1 | GAGTCCT | GA | AGAGGCTGAAGAGGAAGAC | ACACTCTT | TGGAGT |
| OT4 | AGCAGGA | G | CAGACTCAGTAAAGCCTGG | TCCCTACA | TCAGAC |
| | GAAGAA | | AGGCTGCCAGGTAGGGCTG | CGACGCT | GTGTGC |
| | (SEQ ID | | GGGCCAGCATGACCTGAGT | CTTCCGAT | TCTTCCG |
| | NO: 245) | | CCTAGCAGGAGAAGAAGAG | CTNNNNA | ATCTGG |
| | | | GCAGCCTAGAGTCTTCTGTG | GAGGCTG | CCCAGC |
| | | | AAGTGCACATAGAAGAGAG | AAGAGGA | TGTGCA |
| | | | ACTGGGGCCAAGCCACAAA | AGACCA | TTCTAT |
| | | | AGATAGAATGCACAGCTGG | (SEQ ID | (SEQ ID |
| | | | GCC (SEQ ID NO: 113) | NO: 139) | NO: 194) |
| EMX1 | AAGTCTG | TG | GTAGTTCTGACATTCCTCCT | ACACTCTT | TGGAGT |
| OT5 | AGCACAA | G | GAGGGAAAATAAATAAATT | TCCCTACA | TCAGAC |
| | GAAGAA | | AATTAAAAATATATATATAT | CGACGCT | GTGTGC |
| | (SEQ ID | | ATGTATAATGATAAACATGC | CTTCCGAT | TCTTCCG |
| | NO: 246) | | TAACAAAGTCTGAGCACAA | CTNNNNG | ATCTTG |
| | | | GAAGAATGGTGAGAAGGAA | TAGTTCTG | GTCAAT |
| | | | TACATTTTATCTAATAAATA | ACATTCCT | ATCTGA |
| | | | TGTAAGCCATTAATAAAATG | CCTGAG | AAGGTT |
| | | | TAAACCATTAAAACAACAA | (SEQ ID | TATTTGT |
| | | | ATAAACCTTTCAGATATTGA | NO: 140) | (SEQ ID |
| | | | CCA (SEQ ID NO: 114) | | NO: 195) |
| EMX1 | GAGTCCG | AG | CCAAGAGGGCCAAGTCCTG | ACACTCTT | TGGAGT |
| OT6 | GGAAGGA | G | GCTGTCTGCCTCTGACGACG | TCCCTACA | TCAGAC |
| | GAAGAA | | AGCAAGGTGGAGGCCCTTG | CGACGCT | GTGTGC |
| | (SEQ ID | | GTTAGCAGGATGGGTGGTG | CTTCCGAT | TCTTCCG |
| | NO: 247) | | AGGAGTCCGGGAAGGAGAA | CTNNNNC | ATCTCA |
| | | | GAAAGGCTCAGCGCGGCTT | CAAGAGG | GCGAGG |
| | | | GCCTGAGCCTCCCTCCTCCC | GCCAAGT | AGTGAC |
| | | | AGCTCCCGGCCCCTGCTGCC | CCTG (SEQ | AGCC |
| | | | GGCGGCTGTCACTCCTCGCT | ID NO: 141) | (SEQ ID |
| | | | G (SEQ ID NO: 115) | | NO: 196) |
| EMX1 | GAGCCGG | GG | CACTCCACCTGATCTCGGGG | ACACTCTT | TGGAGT |
| OT7 | AGCAGAA | G | CGCTGTGCGCTGAGGAAGG | TCCCTACA | TCAGAC |
| | GAAGGA | | CGCGGGCGAGCCGGAGCAG | CGACGCT | GTGTGC |
| | (SEQ ID | | AAGAAGGAGGGAGGGAGCC | CTTCCGAT | TCTTCCG |
| | NO: 248) | | AGCCGCTGCAGCCACCACC | CTNNNNC | ATCTCG |
| | | | GCCACCATGTCCTACCAAGG | ACTCCACC | AGGAGG |
| | | | CAAGAAGAACATCCCGCGG | TGATCTCG | GAGGGA |
| | | | ATCACGGTGAGTCCGGGCG | GGG (SEQ | GCAG |
| | | | CCGCTGCTCCCTCCCTCCTC | ID NO: 171) | (SEQ ID |
| | | | G (SEQ ID NO: 116) | | NO: 197) |
| EMX1 | AAGTCCG | AG | ACCACAAATGCCCAAGAGA | ACACTCTT | TGGAGT |
| OT8 | AGGAGAG | G | CATCACCACTTGGAGAGTCA | TCCCTACA | TCAGAC |
| | GAAGAA | | GAGGTCACAAAAGAGGGGC | CGACGCT | GTGTGC |
| | (SEQ ID | | CCAACTCCTGTAGAAGTCCG | CTTCCGAT | TCTTCCG |
| | NO: 249) | | AGGAGAGGAAGAAAGGGTT | CTNNNNA | ATCTGA |
| | | | CTGGAGCTCTCAGGCGTCAG | CCACAAA | CACAGT |
| | | | GGCCAGGCCTGCACCCTTCT | TGCCCAA | CAAGGG |
| | | | GTGCCCCTCCATGAATGGCT | GAGAC | CCGG |
| | | | GGCCGGCCCTTGACTGTGTC | (SEQ ID | (SEQ ID |
| | | | (SEQ ID NO: 117) | NO: 172) | NO: 198) |
| EMX1 | GAATCCA | GG | CCCACCTTTGAGGAGGCAA | ACACTCTT | TGGAGT |
| OT9 | AGCAGGA | A | AAGGGAATAAACTTGTGCTT | TCCCTACA | TCAGAC |
| | GAAGAA | | ATTTGTTGGAAGAGCAAATA | CGACGCT | GTGTGC |
| | (SEQ ID | | TGTTTTTTTGAAACCGAATT | CTTCCGAT | TCTTCCG |
| | NO: 250) | | ATGGATGGGGATGTGGGGG | CTNNNNC | ATCTTTC |
| | | | TGGGAACTAGGCAAGGGTC | CCACCTTT | CATCTG |
| | | | TCAGGGGAATCCAAGCAGG | GAGGAGG | AGAAGA |
| | | | AGAAGAAGGAGGGAAAAAC | CAAA (SEQ | GAGTGG |
| | | | CACTCTCTTCTCAGATGGAA | ID NO: 173) | T (SEQ ID |
| | | | (SEQ ID NO: 118) | | NO: 199) |
| EMX1 | ACGTCTG | TG | GTCATACCTTGGCCCTTCCT | ACACTCTT | TGGAGT |
| OT10 | AGCAGAA | G | CTGTACTCTATACAGAGTCC | TCCCTACA | TCAGAC |
| | GAAGAA | | AGCTCTGGCCTGGGAAAAT | CGACGCT | GTGTGC |
| | (SEQ ID | | ACTTTCAGACAAAACGTCTG | CTTCCGAT | TCTTCCG |
| | NO: 251) | | AGCAGAAGAAGAATGGACA | CTNNNNG | ATCTTCC |
| | | | GAACTCTGAGGACATTCTTG | TCATACCT | CTAGGC |
| | | | AGGCACTGGCAGAACCTCT | TGGCCCTT | CCACAC |
| | | | GCAGGAAGACGAGAGCATT | CCT (SEQ | CAG (SEQ |
| | | | GCTGGTGTGGGCCTAGGGA | ID NO: 174) | ID NO: |
| | | | (SEQ ID NO: 119) | | 201) |
| BCL11A | CCTATCA | GG | ACCTGTGGGCATCCTGAGTT | ACACTCTT | TGGAGT |
| OT1 | CTGGCTC | G | GCTTCTGATGTCCCACCCAT | TCCCTACA | TCAGAC |
| | CAGGAA | | CACCTTGACCTGCTCAGAGC | CGACGCT | GTGTGC |
| | (SEQ ID | | AGAGCATTGTTCTGAAATCT | CTTCCGAT | TCTTCCG |
| | NO: 252) | | GAGGCATTGTCCTGCCCACT | CTNNNNA | ATCTTC |
| | | | GGCCTATCACTGGCTCCAGG | CCTGTGG | ACGGCC |
| | | | AAGGGCCTAGTGTCTCTGAC | GCATCCTG | CCACTC |
| | | | CAGCTCTAGATCACCTCCTC | AGTTGC | CTCTCA |
| | | | CTCCTCCTGAGCCCTGTACG | (SEQ ID | (SEQ ID |
| | | | TTGCCAGGCTGATGAGAGG | NO: 175) | NO: 202) |
| | | | AGTGGGGCCGTGA (SEQ ID | | |
| | | | NO: 120) | | |
| BCL11A | CTTATCA | AG | CTTGGCGCAGTTCCTGTGTA | ACACTCTT | TGGAGT |
| OT2 | TAGGCCC | G | TGGATATTCTTACAGAATCG | TCCCTACA | TCAGAC |
| | CAGGAA | | CTACTCTCCCTCTCCTTTGA | CGACGCT | GTGTGC |
| | (SEQ ID | | GCTGGCCTAGCTTTGGCTTA | CTTCCGAT | TCTTCCG |
| | NO: 253) | | TCATAGGCCCCAGGAAAGG | CTNNNNC | ATCTTA |
| | | | CCAGGGGACTGGGGTACCG | TTGGCGC | CATGCT |
| | | | GTTAGAGGGATATAAAAGT | AGTTCCTG | GTGAGA |
| | | | TCATTCTGCCTTGTACGTAT | TGTATG | AAATGA |
| | | | GTTTAATTGATTAGAACACT | (SEQ ID | AGTGT |
| | | | TCATTTTCTCACAGCATGTA | NO: 176) | (SEQ ID |
| | | | (SEQ ID NO: 121) | | NO: 203) |
| CCR5 | TAGGGTG | CA | GACTTCTGGTTATCAGTAGG | ACACTCTT | TGGAGT |
| OT1 | TCAACTC | G | CTGTTAGTAGTTAAGTTTTG | TCCCTACA | TCAGAC |
| | TTGACA | | GGGGAGTCAAAAGTTATAC | CGACGCT | GTGTGC |
| | (SEQ ID | | CTGCATTTGACTGTGTCAAG | CTTCCGAT | TCTTCCG |
| | NO: 254) | | AGTTGACACCCTAACCTCCA | CTNNNNG | ATCTCA |
| | | | CGTTGTTTAAGAGTTAACTG | ACTTCTGG | AAATGG |
| | | | TAGTTTGAACTACACTAGTT | TTATCAGT | ATCATG |
| | | | TGTACTGGTTTGCAGTTATT | AGGCTGTT | GCTCAA |
| | | | TATTATACTATATTCATCAT | (SEQ ID | (SEQ ID |
| | | | TACTTTTATGTTTCAATGAA | NO: 177) | NO: 207) |
| | | | CCATTGTTGAGCCATGATCC | | |
| | | | ATTTTG (SEQ ID NO: 122) | | |
| CCR5 | TTATCTG | AA | TGAGGACTTGGTGAATCGTG | ACACTCTT | TGGAGT |
| OT3 | TCAACTC | T | GTATCATTTGTCAAGAGTTG | TCCCTACA | TCAGAC |
| | TTGACA | | ACAGATAAGAGGAAGGGAG | CGACGCT | GTGTGC |
| | (SEQ ID | | TAGGTTTGTCAGGGAAAGG | CTTCCGAT | TCTTCCG |
| | NO: 255) | | GTTAATTTTTTTTTTTTGGAG | CTNNNNT | ATCTAA |
| | | | ACTTGTTGCCCAGGCTGGAG | GAGGACT | TTAGCT |
| | | | TACAATGGCATGATCTTGGC | TGGTGAA | GGGTGT |
| | | | TCACCACAACTTCCGCCTCC | TCGTGG | GGTGGC |
| | | | TGGGTTCAAGTGATTTTGCT | (SEQ ID | (SEQ ID |
| | | | GCCTCAGCCTCCCGAGTAGC | NO: 178) | NO: 220) |
| | | | TGGGATTACAGGCATGTGCC | | |
| | | | ACCACACCCAGCTAATT | | |
| | | | (SEQ ID NO: 123) | | |
| CCR5 | ATACCTG | TG | CAATTACAGAAAGTGGATT | ACACTCTT | TGGAGT |
| OT8 | CCAACTC | G | GGTTGTGGAATGTACAGATT | TCCCTACA | TCAGAC |
| | TTGACA | | AACAATGGAGAGCACATAA | CGACGCT | GTGTGC |
| | (SEQ ID | | CCCTCAGTCTGGCACACCAG | CTTCCGAT | TCTTCCG |
| | NO: 256) | | TGGAGGCTAAAGAAACTGA | CTNNNNC | ATCTTTG |
| | | | AAGGTGTTCTGCCCAGGGTG | AATTACA | TCTTGA |
| | | | CAGGGACTCCATGTCAAGA | GAAAGTG | GATTTTC |
| | | | GTTGGCAGGTATGGAAAGC | GATTGGTT | CTAATG |
| | | | AAGAGGGTTGGGACAGAAA | G (SEQ ID | ATTT |
| | | | CCAGGGTAATTAATTAAGTG | NO: 179) | (SEQ ID |
| | | | ACTATTTTTAGGACTAACTT | | NO: 221) |
| | | | CAAGGGTTACATGAGCTCCC | | |
| | | | TCCCCTCCAAAAACAAATCA | | |
| | | | TTAGGAAAATCTCA (SEQ ID | | |
| | | | NO: 124) | | |
| CXCR4 | TGTTCTC | TG | TTGCTGAAAGTGACGACAC | ACACTCTT | TGGAGT |
| OT8 | AGCACAT | T | AGCCAGTGAAGCTGGGACT | TCCCTACA | TCAGAC |
| | CATGGT | | GGAATGTCAGTTTCCATAAC | CGACGCT | GTGTGC |
| | (SEQ ID | | TCCAGCCCCCTTTTTTAACC | CTTCCGAT | TCTTCCG |
| | NO: 257) | | GTTAGGAAACATCCTTTCAC | CTNNNNTT | ATCTTG |
| | | | CTCAGGTCCCCTCAGTCTCT | GCTGAAA | CTCAGG |
| | | | AGTCCACACCATGATGTGCT | GTGACGA | AAAAGT |
| | | | GAGAACAGGAGGCATACTA | CACAG | CTTTGG |
| | | | TCAGCATTTTAAAAGGACCT | (SEQ ID | A (SEQ ID |
| | | | CTTCCTTGTTTCTCCTGTTCT | NO: 180) | NO: 222) |
| | | | CTGGCCCCAGAGAGCTCTTC | | |
| | | | TGAGACCCTGGGGTCCAGA | | |
| | | | AGGGAGCCCTGGAGAACTT | | |
| | | | AGCTCCAAAGACTTTTCCTG | | |
| | | | AGCA (SEQ ID NO: 125) | | |

TABLE 2D
SaCas9 orthogonal R-loop sites:

| Site | Proto- | | | | |
|---|---|---|---|---|---|
| Name | spacer | PAM | Amplicon | HTS-F | HTS-R |
| SaR1 | GTGGTA | AA | TGGTGGAGTGCTCTGTGTTTG | ACACTCTT | TGGAGT |
| | GACAGC | GG | TCTTTATAAACCCAGATGAG | TCCCTACA | TCAGAC |
| | ATGTGTC | GT | AGGATGAAGGCAACAAGCTT | CGACGCTC | GTGTGC |
| | CTA (SEQ | | CTGTACCAACATACATGCCC | TTCCGATC | TCTTCC |
| | ID NO: | | CTTTGCCTCAAGTCTGGTTAT | TNNNNTG | GATTGG |
| | 258) | | TTTAGGGGGATGCTAGGTTG | CAGTCTCC | TGGAGT |
| | | | CTTTGGGTCTACCTTACTGAG | TGCTTCTC | GCTCTG |
| | | | AAAATGGCCCCAGGTCATTG | TG (SEQ ID | TGTTTG |
| | | | TCATGTCCAGTTGTGGTAGA | NO: 275) | (SEQ ID |
| | | | CAGCATGTGTCCTAAAGGGT | | NO: 283) |
| | | | ATATTCACATGCATGTGCAA | | |
| | | | AAATACAGGGGTCCTTCTAA | | |
| | | | CCCTATCACAGAGAAGCAGG | | |
| | | | AGACTGC (SEQ ID NO: 223) | | |
| SaR2 | ATTTACA | TC | GCTACAGAAAGGTCAGCAGC | ACACTCTT | TGGAGT |
| | GCCTGG | GG | TATATTTAACCTCAGACCAG | TCCCTACA | TCAGAC |
| | CCTTTGG | GT | GGTGCGGTGGGAGATCTGGT | CGACGCTC | GTGTGC |
| | GG (SEQ | | TTCCGGAAGACGGAATGGGG | TTCCGATC | TCTTCC |
| | ID NO: | | AGAAGGGCAGGTTCCCCGAG | TNNNNGG | GATGCT |
| | 259) | | GCGCCCAGACACCCAATCCT | ACATTTCC | ACAGAA |
| | | | CCCGGTGACATTTACAGCCT | ACCGCAA | AGGTCA |
| | | | GGCCTTTGGGGTCGGGTCAA | AATG (SEQ | GCAGC |
| | | | CGCTAGGCTGGCAGGGGAAG | ID NO: 280) | (SEQ ID |
| | | | GGCGGGGCCGTGAGGTGAGC | | NO: 284) |
| | | | CGGCGCTGCAGGAAGGGGCC | | |
| | | | ACCACCAGAGGGGCCATTTT | | |
| | | | GCGGTGGAAATGTCC (SEQ ID | | |
| | | | NO: 224) | | |
| SaR3 | GTGTCA | GA | AAGTGTTCAGCTGCTTTTCTT | ACACTCTT | TGGAGT |
| | GGTAAT | GA | TCATTTATTCCACATATAATT | TCCCTACA | TCAGAC |
| | GTGCTA | GT | ACTATAATTGCTAAACATTTA | CGACGCTC | GTGTGC |
| | AACA | | TTTAGTGTCAGGTAATGTGCT | TTCCGATC | TCTTCC |
| | (SEQ ID | | AAACAGAGAGTTACTGCTCA | TNNNNCT | GATCTG |
| | NO: 260) | | GACATGTAATAATAATAAAT | GCACCTA | CTGTGG |
| | | | AACACATCAAATAACCATAC | GCCTCCAT | CATCCA |
| | | | CATTTTAAGCTGTAGTATTAT | GTC (SEQ | GAGACA |
| | | | GAAGGGAAATCTGGAGCAAA | ID NO: 281) | T (SEQ ID |
| | | | GAGAATAGACTGTAGGGAAA | | NO: 285) |
| | | | CCAGTTAAGAAATAGGACAT | | |
| | | | GGAGGCTAGGTGCAG (SEQ | | |
| | | | ID NO: 225) | | |
| SaR4 | GGTGGA | CA | TTTGCTTATCCAGAAAAGGG | ACACTCTT | TGGAGT |
| | GGAGGG | GA | AGTGATTGCTTCCAGGGGCC | TCCCTACA | TCAGAC |
| | TGCATG | AT | TCAGGGGAATAAATCATAGA | CGACGCTC | GTGTGC |
| | GGGT | | ATCCTGGACAAGGTTTGAAG | TTCCGATC | TCTTCC |
| | (SEQ ID | | GACAGGTAGGATTTGGGTGG | TNNNNGG | GATCTT |
| | NO: 261) | | GTGGAGGAGGGTGCATGGGG | AGGTGGA | CCTGAG |
| | | | TCAGAATTGTAACCGAAAAC | GAGAGGA | GTCTAG |
| | | | TCATTCCAGGTGGATAGAGA | TGT (SEQ | GAACCC |
| | | | AAATTTCTAGTGTTGTTGTTT | ID NO: 282) | G (SEQ ID |
| | | | TTAAACTATTTGGGGGACTG | | NO: 286) |
| | | | GCACAGACCCTTTTTGAATA | | |
| | | | CCTGATGGGCTCACATTTCTG | | |
| | | | TCGAATCCCAG (SEQ ID NO: | | |
| | | | 273) | | |
| SaR5 | TCTGCTT | CT | ATGTGGGCTGCCTAGAAAGG | ACACTCTT | TGGAGT |
| | CTCCAG | GC | CATGGATGAGAGAAGCCTGG | TCCCTACA | TCAGAC |
| | CCCTGG | GT | AGACAGGGATCCCAGGGAAA | CGACGCTC | GTGTGC |
| | C (SEQ ID | | CGCCCATGCAATTAGTCTATT | TTCCGATC | TCTTCC |
| | NO: 262) | | TCTGCTGCAAGTAAGCATGC | TNNNNAT | GATCTC |
| | | | ATTTGTAGGCTTGATGCTTTT | GTGGGCT | CCAGCC |
| | | | TTTCTGCTTCTCCAGCCCTGG | GCCTAGA | AAACTT |
| | | | CCTGGGTCAATCCTTGGGGC | AAGG (SEQ | GTCAAC |
| | | | CCAGACTGAGCACGTGATGG | ID NO: 57) | C (SEQ ID |
| | | | CAGAGGAAAGGAAGCCCTGC | | NO: 69) |
| | | | TTCCTCCAGAGGGCGTCGCA | | |
| | | | GGACAGCTTTTCCTAGACAG | | |
| | | | GGGCTAGTATGTGCAGCTCC | | |
| | | | TGCACCGGGATACTGGTTGA | | |
| | | | CAAGTTTGGCTGGG (SEQ ID | | |
| | | | NO: 10) | | |
| SaR6 | GATGTTC | GA | CATTGCAGAGAGGCGTATCA | ACACTCTT | TGGAGT |
| | CAATCA | GA | TTTCGCGGATGTTCCAATCAG | TCCCTACA | TCAGAC |
| | GTACGC | GT | TACGCAGAGAGTCGCCGTCT | CGACGCTC | GTGTGC |
| | A (SEQ ID | | CCAAGGTGAAAGCGGAAGTA | TTCCGATC | TCTTCC |
| | NO: 263) | | GGGCCTTCGCGCACCTCATG | TNNNNCA | GATCTG |
| | | | GAATCCCTTCTGCAGCACCT | TTGCAGA | GGGTCC |
| | | | GGATCGCTTTTCCGAGCTTCT | GAGGCGT | CAGGTG |
| | | | GGCGGTCTCAAGCACTACCT | ATCA (SEQ | CTGAC |
| | | | ACGTCAGCACCTGGGACCCC | ID NO: 32) | (SEQ ID |
| | | | (SEQ ID NO: 7) | | NO: 66) |

TABLE 2E
SaCas9 genomic loci:

| SITE | PROTO- | | | | |
|---|---|---|---|---|---|
| NAME | SPACER | PAM | AMPLICON | HTS-F | HTS-R |
| SA1 | GTGGTAG | AA | TGGTGGAGTGCTCTGTGTTTG | ACACTCT | TGGAGT |
| | ACAGCAT | GG | TCTTTATAAACCCAGATGAGA | TTCCCTA | TCAGAC |
| | GTGTCCT | GT | GGATGAAGGCAACAAGCTTC | CACGACG | GTGTGC |
| | A (SEQ ID | | TGTACCAACATACATGCCCCT | CTCTTCC | TCTTCC |
| | NO: 258) | | TTGCCTCAAGTCTGGTTATTT | GATCTNN | GATTGG |
| | | | TAGGGGGATGCTAGGTTGCTT | NNTGCAG | TGGAGT |
| | | | TGGGTCTACCTTACTGAGAA | TCTCCTG | GCTCTG |
| | | | AATGGCCCCAGGTCATTGTCA | CTTCTCT | TGTTTG |
| | | | TGTCCAGTTGTGGTAGACAG | G (SEQ ID | (SEQ ID |
| | | | CATGTGTCCTAAAGGGTATAT | NO: 275) | NO: 283) |
| | | | TCACATGCATGTGCAAAAATA | | |
| | | | CAGGGGTCCTTCTAACCCTAT | | |
| | | | CACAGAGAAGCAGGAGACT | | |
| | | | GC (SEQ ID NO: 223) | | |
| SA2 | ATTTACA | TC | GCTACAGAAAGGTCAGCAGC | ACACTCT | TGGAGT |
| | GCCTGGC | GG | TATATTTAACCTCAGACCAGG | TTCCCTA | TCAGAC |
| | CTTTGGG | GT | GTGCGGTGGGAGATCTGGTT | CACGACG | GTGTGC |
| | G (SEQ ID | | TCCGGAAGACGGAATGGGGA | CTCTTCC | TCTTCC |
| | NO: 259) | | GAAGGGCAGGTTCCCCGAGG | GATCTNN | GATGCT |
| | | | CGCCCAGACACCCAATCCTC | NNGGACA | ACAGAA |
| | | | CCGGTGACATTTACAGCCTGG | TTTCCAC | AGGTCA |
| | | | CCTTTGGGGTCGGGTCAACG | CGCAAAA | GCAGC |
| | | | CTAGGCTGGCAGGGGAAGGG | TG (SEQ | (SEQ ID |
| | | | CGGGGCCGTGAGGTGAGCCG | ID NO: | NO: 284) |
| | | | GCGCTGCAGGAAGGGGCCAC | 280) | |
| | | | CACCAGAGGGGCCATTTTGC | | |
| | | | GGTGGAAATGTCC (SEQ ID | | |
| | | | NO: 224) | | |
| SA3 | GTGTCAG | GA | AAGTGTTCAGCTGCTTTTCTT | ACACTCT | TGGAGT |
| | GTAATGT | GA | TCATTTATTCCACATATAATTA | TTCCCTA | TCAGAC |
| | GCTAAAC | GT | CTATAATTGCTAAACATTTATT | CACGACG | GTGTGC |
| | A (SEQ ID | | TAGTGTCAGGTAATGTGCTAA | CTCTTCC | TCTTCC |
| | NO: 260) | | ACAGAGAGTTACTGCTCAGA | GATCTNN | GATCTG |
| | | | CATGTAATAATAATAAATAAC | NNCTGCA | CTGTGG |
| | | | ACATCAAATAACCATACCATT | CCTAGCC | CATCCA |
| | | | TTAAGCTGTAGTATTATGAAG | TCCATGT | GAGACA |
| | | | GGAAATCTGGAGCAAAGAGA | C (SEQ ID | T (SEQ |
| | | | ATAGACTGTAGGGAAACCAG | NO: 281) | ID NO: |
| | | | TTAAGAAATAGGACATGGAG | | 66) |
| | | | GCTAGGTGCAG (SEQ ID NO: | | |
| | | | 225) | | |
| SA4 | TCTGCTT | CT | ATGTGGGCTGCCTAGAAAGG | ACACTCT | TGGAGT |
| | CTCCAGC | GG | CATGGATGAGAGAAGCCTGG | TTCCCTA | TCAGAC |
| | CCTGGC | GT | AGACAGGGATCCCAGGGAAA | CACGACG | GTGTGC |
| | (SEQ ID | | CGCCCATGCAATTAGTCTATT | CTCTTCC | TCTTCC |
| | NO: 262) | | TCTGCTGCAAGTAAGCATGCA | GATCTNN | GATCTC |
| | | | TTTGTAGGCTTGATGCTTTTT | NNATGTG | CCAGCC |
| | | | TTCTGCTTCTCCAGCCCTGGC | GGCTGCC | AAACTT |
| | | | CTGGGTCAATCCTTGGGGCC | TAGAAAG | GTCAAC |
| | | | CAGACTGAGCACGTGATGGC | G (SEQ ID | C (SEQ |
| | | | AGAGGAAAGGAAGCCCTGCT | NO: 57) | ID NO: |
| | | | TCCTCCAGAGGGCGTCGCAG | | 69) |
| | | | GACAGCTTTTCCTAGACAGG | | |
| | | | GGCTAGTATGTGCAGCTCCTG | | |
| | | | CACCGGGATACTGGTTGACA | | |
| | | | AGTTTGGCTGGG (SEQ ID NO: | | |
| | | | 10) | | |
| SA5 | GATGTTC | GA | CATTGCAGAGAGGCGTATCAT | ACACTCT | TGGAGT |
| | CAATCAG | GA | TTCGCGGATGTTCCAATCAGT | TTCCCTA | TCAGAC |
| | TACGCA | GT | ACGCAGAGAGTCGCCGTCTC | CACGACG | GTGTGC |
| | (SEQ ID | | CAAGGTGAAAGCGGAAGTAG | CTCTTCC | TCTTCC |
| | NO: 263) | | GGCCTTCGCGCACCTCATGG | GATCTNN | GATCTG |
| | | | AATCCCTTCTGCAGCACCTGG | NNCATTG | GGGTCC |
| | | | ATCGCTTTTCCGAGCTTCTGG | CAGAGAG | CAGGTG |
| | | | CGGTCTCAAGCACTACCTAC | GCGTATC | CTGAC |
| | | | GTCAGCACCTGGGACCCC | A (SEQ ID | (SEQ ID |
| | | | (SEQ ID NO: 7) | NO: 32) | NO: 72) |
| SA6 | GCAAGGC | CG | GATCGCTTTTCCGAGCTTCTG | ACACTCT | TGGAGT |
| | CCGGCGC | GG | GCGGTCTCAAGCACTACCTA | TTCCCTA | TCAGAC |
| | ACGGTGG | GT | CGTCAGCACCTGGGACCCCG | CACGACG | GTGTGC |
| | (SEQ ID | | CCACCGTGCGCCGGGCCTTG | CTCTTCC | TCTTCC |
| | NO: 291) | | CAGTGGGCGCGCTACCTGCG | GATCTNN | GATCTG |
| | | | CCACATCCATCGGCGCTTTGG | NNGATCG | CACCAG |
| | | | TCGGCATGGCCCCATTCGCAC | CTTTTCC | GTGGTA |
| | | | GGCTCTGGAGCGGCGGCTGC | GAGCTTC | ACGAGC |
| | | | ACAACCAGTGGAGGCAAGA | TGGC | TGCATC |
| | | | GGGCGGCTTTGGGCGGGGTC | (SEQ ID | (SEQ ID |
| | | | CAGTTCCGGGATTAGCGAAC | NO: 304) | NO: 287) |
| | | | TTCCAGGCCCTCGGTCACTGT | | |
| | | | GACGTCCTGCTCTCTCTGCGC | | |
| | | | CTGCTGGAGAACCGGGCCCT | | |
| | | | CGGGGATGCAGCTCGTTACC | | |
| | | | ACCTGGTGC (SEQ ID NO: 299) | | |
| SA7 | ACGTGCT | AA | ATGTGGGCTGCCTAGAAAGG | ACACTCT | TGGAGT |
| | CAGTCTG | GG | CATGGATGAGAGAAGCCTGG | TTCCCTA | TCAGAC |
| | GGCCCC | AT | AGACAGGGATCCCAGGGAAA | CACGACG | GTGTGC |
| | (SEQ ID | | CGCCCATGCAATTAGTCTATT | CTCTTCC | TCTTCC |
| | NO: 292) | | TCTGCTGCAAGTAAGCATGCA | GATCTNN | GATCTC |
| | | | TTTGTAGGCTTGATGCTTTTT | NNATGTG | CCAGCC |
| | | | TTCTGCTTCTCCAGCCCTGGC | GGCTGCC | AAACTT |
| | | | CTGGGTCAATCCTTGGGGCC | TAGAAAG | GTCAAC |
| | | | CAGACTGAGCACGTGATGGC | G (SEQ ID | C (SEQ |
| | | | AGAGGAAAGGAAGCCCTGCT | NO: 57) | ID NO: |
| | | | TCCTCCAGAGGGCGTCGCAG | | 285) |
| | | | GACAGCTTTTCCTAGACAGG | | |
| | | | GGCTAGTATGTGCAGCTCCTG | | |
| | | | CACCGGGATACTGGTTGACA | | |
| | | | AGTTTGGCTGGG (SEQ ID NO: | | |
| | | | 10) | | |
| SA8 | GCGAGGC | CA | GAACCCAGGTAGCCAGAGAC | ACACTCT | TGGAGT |
| | AGAGGGT | GG | CCGCTGGTCTTCTTTCCCCTC | TTCCCTA | TCAGAC |
| | CCAAAG | AT | CCCTGCCCTCCCCTCCCTTCA | CACGACG | GTGTGC |
| | (SEQ ID | | AGATGGCTGACAAAGGCCGG | CTCTTCC | TCTTCC |
| | NO: 293) | | GCTGGGTGGAAGGAAGGGA | GATCTNN | GATCTT |
| | | | GGAAGGGCGAGGCAGAGGG | NNGAACC | CCTTTC |
| | | | TCCAAAGCAGGATGACAGGC | CAGGTAG | AACCCG |
| | | | AGGGGCACCGCGGCGCCCCG | CCAGAGA | AACGGA |
| | | | GTGGCACTGCGGCTGGAGGT | C (SEQ ID | G (SEQ |
| | | | GGGGGTTAAAGCGGAGACTC | NO: 60) | ID NO: |
| | | | TGGTGCTGTGTGACTACAGT | | 72) |
| | | | GGGGGCCCTGCCCTCTCTGA | | |
| | | | GCCCCCGCCTCCAGGCCTGT | | |
| | | | GTGTGTGTCTCCGTTCGGGTT | | |
| | | | GAAAGGA (SEQ ID NO: 13) | | |
| SA9 | CCACTGT | CA | GAACCCAGGTAGCCAGAGAC | ACACTCT | TGGAGT |
| | AGTCACA | GA | CCGCTGGTCTTCTTTCCCCTC | TTCCCTA | TCAGAC |
| | CAGCAC | GT | CCCTGCCCTCCCCTCCCTTCA | CACGACG | GTGTGC |
| | (SEQ ID | | AGATGGCTGACAAAGGCCGG | CTCTTCC | TCTTCC |
| | NO: 294) | | GCTGGGTGGAAGGAAGGGA | GATCTNN | GATCTT |
| | | | GGAAGGGCGAGGCAGAGGG | NNGAACC | CCTTTC |
| | | | TCCAAAGCAGGATGACAGGC | CAGGTAG | AACCCG |
| | | | AGGGGCACCGCGGCGCCCCG | CCAGAGA | AACGGA |
| | | | GTGGCACTGCGGCTGGAGGT | C (SEQ ID | G (SEQ |
| | | | GGGGGTTAAAGCGGAGACTC | NO: 60) | ID NO: |
| | | | TGGTGCTGTGTGACTACAGT | | 72) |
| | | | GGGGGCCCTGCCCTCTCTGA | | |
| | | | GCCCCCGCCTCCAGGCCTGT | | |
| | | | GTGTGTGTCTCCGTTCGGGTT | | |
| | | | GAAAGGA (SEQ ID NO: 13) | | |

TABLE 3
cDNA amplicon sequences and primers for RNA off-target analysis.

| Name | Amplicon | HTS-F | HTS-R |
|---|---|---|---|
| RSL1D1 | TTGGCTTTCCAAATCAGTGGGTCTGACTTG | ACACTCTT | TGGAGTT |
| | AGGTCTGTGATGTGACCCTTTTCCTCACCT | TCCCTACA | CAGACGT |
| | GCTCAACCATTATTCACATGGACTCCATCA | CGACGCTC | GTGCTCTT |
| | TATTCATTTGTAGTCATTCCCAGAGTGGCC | TTCCGATC | CCGATCT |
| | CAGTGAGGGTCTCGCTGTATGAGAGTCGG | TNNNNTGG | CTCATAA |
| | CTACGGAATTTAGGAGAAACAGAAGTTTC | CTTTCCAA | GCTTAGA |
| | TTGGCTTTCATGCTGAGCTTGTTGGTCTAA | ATCAGTGG | CCAACAA |
| | GCTTATGAG (SEQ ID NO: 288) | GTC (SEQ ID | GC (SEQ ID |
| | | NO: 295) | NO: 298) |
| CTNNB1 | TTTGATGGAGTTGGACATGGCCATGGAAC | ACACTCTT | TGGAGTT |
| | CAGACAGAAAAGCGGCTGTTAGTCACTGG | TCCCTACA | CAGACGT |
| | CAGCAACAGTCTTACCTGGACTCTGGAATC | CGACGCTC | GTGCTCTC |
| | CATTCTGGTGCCACTACCACAGCTCCTTCT | TTCCGATC | CAGCTAC |
| | CTGAGTGGTAAAGGCAATCCTGAGGAAGA | TNNNNATT | TTGTTCTT |
| | GGATGTGGATACCTCCCAAGTCCTGTATGA | TGATGGAG | GAGTGAA |
| | GTGGGAACAGGGATTTTCTCAGTCCTTCAC | TTGGACAT | GG (SEQ ID |
| | TCAAGAACAAGTAGCTGG (SEQ ID NO: 289) | GGCC (SEQ | NO: 300) |
| | | ID NO: 296) | |
| IP90 | CTGGTTGACCAATCTGTGGTGAATAGTGGA | ACACTCTT | TGGAGTT |
| | AATCTGCTCAATGACATGACTCCTCCTGTA | TCCCTACA | CAGACGT |
| | AATCCTTCACGTGAAATTGAGGACCCAGA | CGACGCTC | GTGCTCTC |
| | AGACCGGAAGCCCGAGGATTGGGATGAAA | TTCCGATC | TGCGTCT |
| | GACCAAAAATCCCAGATCCAGAAGCTGTC | TNNNNCTG | GGATCAG |
| | AAGCCAGATGACTGGGATGAAGATGCCCC | GTTGACCA | GTACG |
| | TGCTAAGATTCCAGATGAAGAGGCCACAA | ATCTGTGG | (SEQ ID |
| | AACCCGAAGGCTGGTTAGATGATGAGCCT | TG (SEQ ID | NO: 301) |
| | GAGTACGTAC (SEQ ID NO: 290) | NO: 297) | |

TABLE 4
Primers for generating base editor
amplicons for IVT.

| Name | Sequence (5′-3′) |
|---|---|
| IVT-F | TCGAGCTCGGTACCTAATACGACTCACTATAAGG (SEQ ID |
| | NO: 302) |
| IVT-R | TTTTTTTTTTTTTTTTTTTTTTTTTTTTTTTTTTTTTTTTT |
| | TTTTTTTTTTTTTTTTTTTTTTTTTTTTTTTTTTTTTTTTT |
| | TTTTTTTTTTTTTTTTTTTTTTTTTTTTTTTTTTTTTCTTC |
| | CTACTCAGGCTTTATTCAAAGACCA (SEQ ID NO: 303) |

TABLE 5
Chemically synthesized guide RNAs used for T cell and HSC
experiments.

| Site/Guide | Protospacer sequence | |
|---|---|---|
| ID* | (5′-3′) | sgRNA scaffold (5′-3′) |
| CCR5 | CAATGTGTCAACTCTTGACA | SpCas9 sgRNA EZ Kit scaffold from |
| | (SEQ ID NO: 217) | Synthego (with 2′-O-Methyl and 3′ |
| | | phosphorothioate bond modifications) |
| CXCR4 | CAGTTTCAGCACATCATGGT | SpCas9 sgRNA EZ Kit scaffold from |
| | (SEQ ID NO: 218) | Synthego (with 2′-O-Methyl and 3′ |
| | | phosphorothioate bond modifications) |
| BCL11A | TTTATCACAGGCTCCAGGAA | SpCas9 sgRNA EZ Kit scaffold from |
| | (SEQ ID NO: 210) | Synthego (with 2′-O-Methyl and 3′ |
| | | phosphorothioate bond modifications) |

*See Table 1 for the list of target sites

Table 6. Selectivity of TadCBEs and TadDE calculated from the mESC library experiment. Selectivity is defined as the geometric mean of (the ratio of (average CBE editing at each position) to (average ABE editing at each position)) for bases in the 30% window. P(ABE|CBE) is the average probability of observing A·T-to-G·C editing in a read given that C·G-to-G·C editing was observed.

| Variant | Selectivity | P (ABE\|CBE) rep1 | P (ABE\|CBE) rep2 |
|---|---|---|---|
| BE4max | >100 | 0.01 | 0.00 |
| TadCBEa | 18.19 | 0.10 | 0.11 |
| TadCBEb | 10.98 | 0.17 | 0.18 |
| TadCBEc | 13.84 | 0.12 | 0.14 |
| TadCBEd | 26.87 | 0.09 | 0.09 |
| TadCBEe | 15.72 | 0.12 | 0.13 |
| TadCBEa | 31.62 | 0.06 | 0.05 |
| V106W | | | |
| TadCBEb | 19.91 | 0.08 | 0.09 |
| V106W | | | |
| TadCBEc | 28.80 | 0.06 | 0.06 |
| V106W | | | |
| TadCBEd | 47.80 | 0.04 | 0.05 |
| V106W | | | |
| TadDE | 1.00 | 0.62 | 0.63 |

TABLE 7
Promoter and RBS sequences

| Name | Promoter/RBS | Description | Sequence |
|---|---|---|---|
| SP | PgIII | Promoter for | AATTCACCTCGAAAGCAAGTTGATA |
| | | TadA*-NpuN | AACTGATACAATTAAAGGCTCCT |
| | | on phage | (SEQ ID NO: 305) |
| SP | RBS | RBS for | AAGGAGGAAAA (SEQ ID NO: 306) |
| | | expression of | |
| | | TadA*-NpuN | |
| | | on phage | |
| P1 | ProC | Promoter for | CACAGCTAACACCACGTCGTCCCTA |
| | | NpuC-dCas9- | TCTGCTGCCCTAGGTCTATGAGTGGT |
| | | ugi | TGCTGGATAACTTTACGGGCATGCA |
| | | | TAAGGCTCGTATGATATATTCAGGG |
| | | | AGACCACAACGGTTTCCCTCTACAA |
| | | | ATAATTTTGTTTAACTTTTACTAGAG |
| | | | TGGGACCCTACCTGCAGGTGCAGT |
| | | | (SEQ ID NO: 307) |
| P1 | SD8 | RBS for | AAGGAGGAAAAAAAA (SEQ ID NO: |
| | | expression of | 308) |
| | | NpuC-dCas9- | |
| | | ugi | |
| P2 | pLac | Promoter for | GGCTTTACACTTTATGCTTCCGGCTC |
| | | sgRNA | GTATGTTGTGTGG (SEQ ID NO: 309) |
| P2 | proT7 | T7 promoter | TAATACGACTCACTATAGGGAGA |
| | | controls gIII | (SEQ ID NO: 310) |
| | | transcription | |
| P2 | sd8 | RBS for | AAGGAAAAAAAAA (SEQ ID NO: 311) |
| | | expression of | |
| | | gIII | |
| P3 | ProA | Promoter for | CACAGCTAACACCACGTCGTCCCTA |
| | | T7RNAP. | TCTGCTGCCCTAGGTCTATGAGTGGT |
| | | Varies by | TGCTGGATAACTTTACGGGCATGCA |
| | | stringency (A is | TAAGGCTCGTAGGCTATATTCAGGG |
| | | weakest, most | AGACCACAACGGTTTCCCTCTACAA |
| | | stringent) | ATAATTTTGTTTAACTTTTACTAGAG |
| | | | TGGGACCCTACCTGCAGGTGCAGT |
| | | | (SEQ ID NO: 312) |
| P3 | ProB | Promoter for | CACAGCTAACACCACGTCGTCCCTA |
| | | T7RNAP. | TCTGCTGCCCTAGGTCTATGAGTGGT |
| | | Varies by | TGCTGGATAACTTTACGGGCATGCA |
| | | stringency. | TAAGGCTCGTAATATATATTCAGGG |
| | | | AGACCACAACGGTTTCCCTCTACAA |
| | | | ATAATTTTGTTTAACTTTTACTAGAG |
| | | | TGGGACCCTACCTGCAGGTGCAGT |
| | | | (SEQ ID NO: 313) |
| P3 | ProC | Promoter for | CACAGCTAACACCACGTCGTCCCTA |
| | | T7RNAP. | TCTGCTGCCCTAGGTCTATGAGTGGT |
| | | Varies by | TGCTGGATAACTTTACGGGCATGCA |
| | | stringency. | TAAGGCTCGTATGATATATTCAGGG |
| | | | AGACCACAACGGTTTCCCTCTACAA |
| | | | ATAATTTTGTTTAACTTTTACTAGAG |
| | | | TGGGACCCTACCTGCAGGTGCAGT |
| | | | (SEQ ID NO: 307) |
| P3 | ProD | Promoter for | CACAGCTAACACCACGTCGTCCCTA |
| | | T7RNAP. | TCTGCTGCCCTAGGTCTATGAGTGGT |
| | | Varies by | TGCTGGATAACTTTACGGGCATGCA |
| | | stringency (D is | TAAGGCTCGTATAATATATTCAGGG |
| | | strongest, least | AGACCACAACGGTTTCCCTCTACAA |
| | | stringent) | ATAATTTTGTTTAACTTTTACTAGAG |
| | | | TGGGACCCTACCTGCAGGTGCAGT |
| | | | (SEQ ID NO: 314) |
| P3 | RBS_{R3} | RBS for | ACTACATCATCAGGC (SEQ ID NO: |
| | | expression of | 316) |
| | | T7 RNAP | |

Table 8. Primers for PCR1 and PCR2 in mESc 12kChar library analysis of base editors. Samples for the library analysis (11 samples with two biological replicates for 22 samples total) were amplified and barcoded for analysis. 4 NextSeq runs were performed with up to 7 samples on each. Primers were assigned to be unique among samples on each run. Run 1 contained 7 samples, barcoded with FWD PCR1A and REV PCR2-1 through PCR2-7. Run 2 contained 7 samples, barcoded with FWD PCR1B and REV PCR2-1 through PCR2-7. Run 3 contained 7 samples, barcoded with FWD PCR1C and REV PCR2-1 through PCR2-7. Run 4 contained 1 sample, barcoded with FWD PCR1D and REV PCR2-1.

| Name | Purpose | Sequence |
|---|---|---|
| Fwd | Library | AATGATACGGCGACCACCGAGATCTACACATTACTCGACACTC |
| PCR1 | Fwd | TTTCCCTACACGAC (SEQ ID NO: 324) |
| A | PCR1 | |
| Fwd | Library | AATGATACGGCGACCACCGAGATCTACACTCCGGAGAACACTC |
| PCR1 | Fwd | TTTCCCTACACGAC (SEQ ID NO: 327) |
| B | PCR1 | |
| Fwd | Library | AATGATACGGCGACCACCGAGATCTACACCGCTCATTACACTC |
| PCR1 | Fwd | TTTCCCTACACGAC (SEQ ID NO: 328) |
| C | PCR1 | |
| Fwd | Library | AATGATACGGCGACCACCGAGATCTACACGAGATTCCACACTC |
| PCR1 | Fwd | TTTCCCTACACGAC (SEQ ID NO: 329) |
| D | PCR1 | |
| Rev | Library | GTGACTGGAGTTCAGACGTGTGCTCTTC |
| PCR1 | Rev | CGATCTGTGGAAAGGACGAAACACCG (SEQ ID NO: 330) |
| Lib | PCR1 | |
| Fwd | Library | AATGATACGGCGACCACCGAGATCTACAC (SEQ ID NO: 331) |
| PCR2 | Fwd | |
| Lib | PCR2 | |
| Rev | Library | CAAGCAGAAGACGGCATACGAGATATCGTGATGTGACTGGAG |
| PCR2- | Rev | TTCAGACGTGTGCT (SEQ ID NO: 332) |
| 1 | PCR2 | |
| Rev | Library | CAAGCAGAAGACGGCATACGAGATATACATCGGTGACTGGAG |
| PCR2- | Rev | TTCAGACGTGTGCT (SEQ ID NO: 333) |
| 2 | PCR2 | |
| Rev | Library | CAAGCAGAAGACGGCATACGAGATATGCCTAAGTGACTGGAG |
| PCR2- | Rev | TTCAGACGTGTGCT (SEQ ID NO: 334) |
| 3 | PCR2 | |
| Rev | Library | CAAGCAGAAGACGGCATACGAGATATTGGTCAGTGACTGGAG |
| PCR2- | Rev | TTCAGACGTGTGCT (SEQ ID NO: 335) |
| 4 | PCR2 | |
| Rev | Library | CAAGCAGAAGACGGCATACGAGATATCACTGTGTGACTGGAG |
| PCR2- | Rev | TTCAGACGTGTGCT (SEQ ID NO: 336) |
| 5 | PCR2 | |
| Rev | Library | CAAGCAGAAGACGGCATACGAGATATATTGGCGTGACTGGAG |
| PCR2- | Rev | TTCAGACGTGTGCT (SEQ ID NO: 337) |
| 6 | PCR2 | |
| Rev | Library | CAAGCAGAAGACGGCATACGAGATATGATCTGGTGACTGGAG |
| PCR2- | Rev | TTCAGACGTGTGCT (SEQ ID NO: 338) |
| 7 | PCR2 | |

TABLE 9
Sequence of Cas9 components of the base editors described herein

| Name | Sequence |
|---|---|
| SpCas9 D10A | DKKYSIGLAIGTNSVGWAVITDEYKVPSKKFKVLGNTDRHSIKKNLIG |
| | ALLFDSGETAEATRLKRTARRRYTRRKNRICYLQEIFSNEMAKVDDSF |
| | FHRLEESFLVEEDKKHERHPIFGNIVDEVAYHEKYPTIYHLRKKLVDS |
| | TDKADLRLIYLALAHMIKFRGHFLIEGDLNPDNSDVDKLFIQLVQTYN |
| | QLFEENPINASGVDAKAILSARLSKSRRLENLIAQLPGEKKNGLFGNLI |
| | ALSLGLTPNFKSNFDLAEDAKLQLSKDTYDDDLDNLLAQIGDQYADL |
| | FLAAKNLSDAILLSDILRVNTEITKAPLSASMIKRYDEHHQDLTLLKAL |
| | VRQQLPEKYKEIFFDQSKNGYAGYIDGGASQEEFYKFIKPILEKMDGT |
| | EELLVKLNREDLLRKQRTFDNGSIPHQIHLGELHAILRRQEDFYPFLKD |
| | NREKIEKILTFRIPYYVGPLARGNSRFAWMTRKSEETITPWNFEEVVD |
| | KGASAQSFIERMTNFDKNLPNEKVLPKHSLLYEYFTVYNELTKVKYV |
| | TEGMRKPAFLSGEQKKAIVDLLFKTNRKVTVKQLKEDYFKKIECFDS |
| | VEISGVEDRFNASLGTYHDLLKIIKDKDFLDNEENEDILEDIVLTLTLFE |
| | DREMIEERLKTYAHLFDDKVMKQLKRRRYTGWGRLSRKLINGIRDK |
| | QSGKTILDFLKSDGFANRNFMQLIHDDSLTFKEDIQKAQVSGQGDSLH |
| | EHIANLAGSPAIKKGILQTVKVVDELVKVMGRHKPENIVIEMARENQT |
| | TQKGQKNSRERMKRIEEGIKELGSQILKEHPVENTQLQNEKLYLYYLQ |
| | NGRDMYVDQELDINRLSDYDVDHIVPQSFLKDDSIDNKVLTRSDKNR |
| | GKSDNVPSEEVVKKMKNYWRQLLNAKLITQRKFDNLTKAERGGLSE |
| | LDKAGFIKRQLVETRQITKHVAQILDSRMNTKYDENDKLIREVKVITL |
| | KSKLVSDFRKDFQFYKVREINNYHHAHDAYLNAVVGTALIKKYPKLE |
| | SEFVYGDYKVYDVRKMIAKSEQEIGKATAKYFFYSNIMNFFKTEITLA |
| | NGEIRKRPLIETNGETGEIVWDKGRDFATVRKVLSMPQVNIVKKTEV |
| | QTGGFSKESILPKRNSDKLIARKKDWDPKKYGGFDSPTVAYSVLVVA |
| | KVEKGKSKKLKSVKELLGITIMERSSFEKNPIDFLEAKGYKEVKKDLII |
| | KLPKYSLFELENGRKRMLASAGELQKGNELALPSKYVNFLYLASHYE |
| | KLKGSPEDNEQKQLFVEQHKHYLDEIIEQISEFSKRVILADANLDKVLS |
| | AYNKHRDKPIREQAENIIHLFTLTNLGAPAAFKYFDTTIDRKRYTSTKE |
| | VLDATLIHQSITGLYETRIDLSQLGGD (SEQ ID NO: 340) |
| eNme2-C Cas9 | AAFKPNPINYILGLAIGIASVGWAMVEIDEEENPIRLIDLGVRVFERAE |
| (S6P mutation | VPKTGDSLAMARRLARSVRRLTRRRAHRLLRARRLLKREGVLQAAD |
| relative to reported | FDENGLIKSLPNTPWQLRAAALDRKLTPLEWSAVLLHLIKHRGYLSQ |
| eNme2-C sequence) | RKNEGETADKELGALLKGVANNAHALQTGDFRTPAELALNKFEKES |
| | GHIRNQRGDYSHTFSRKDLQAELILLFEKQKEFGNPHVSGGLKEGIET |
| | LLMTQRPALSGDAVQKMLGHCTFEPAEPKAAKNTYTAERFIWLTKL |
| | NNLRILEQGSERPLTDTERATLMDEPYRKSKLTYAQARKLLGLEDTAF |
| | FKGLRYGKDNAEASTLMEMKAYHAISRALEKEGLKDKKSPLNLSSEL |
| | QDEIGTAFSLFKTDEDITGRLKDRVQPEILEALLKHISFDKFVQISLKAL |
| | RRIVPLMEQGKRYDEACAEIYGDHYGKKNTEEKIYLPPIPADEIRNPV |
| | VLRALSQARKVINGVVRRYGSPARIHIETAREVGKSFKDRKEIEKRQE |
| | ENRKDREKAAAKFREYFPNFVGEPKSKDILKLRLYEQQHGKCLYSGK |
| | EINLVRLNEKGYVEIDAALPFSRTWDDSFNNKVLVLGSENQNKGNQT |
| | PYEYFNGKDNSREWQEFKARVETSRFPRSKKQRILLQKFDEDGFKEC |
| | NLNDTRYVNRFLCQFVADHILLTGKGKRRVFASNGQITNLLRGFWGL |
| | RKVRAENDRHHALDAVVVACSTVAMQQKITRFVRYKEMNAFDGKTI |
| | DKETGKVLHQKTHFPQPWEFFAQEVMIRVFGKPDGKPEFEEADTPEK |
| | LRTLLAEKLSSRPEAVHEYVTPLFVSRAPNRKMSGAHKDTLRSAKRF |
| | VKHNEKISVKRVWLTEIKLADLENMVNYKNGREIELYEALKARLEAY |
| | GGNAKQAFDPKDNPFYKKGGQLVKAVRVEKTQESGVLLNKKNAYTI |
| | ADNGDMVRVDVFCKVDKKGKNQYFIVPIYAWQVAENILPDIDCKGY |
| | RIDDSYTFCFSLHKYDLIAFQKDEKSKVEFAYYINCDSSNGRFYLAWH |
| | DKGSKEQQFRISTQNLVLIQKYQVNELGKEIRPCRLKKRPPV (SEQ ID |
| | NO: 341) |
| SaCas9 D10A | GKRNYILGLAIGITSVGYGIIDYETRDVIDAGVRLFKEANVENNEGRRS |
| | KRGARRLKRRRRHRIQR VKKLLFDYNLLTDHSELSGINPYEARVKGL |
| | SQKLSEEEFSAALLHLAKRRGVHNVNEVEEDTGNELSTKEQISRNSKA |
| | LEEKYVAELQLERLKKDGEVRGSINRFKTSDYVKEAKQLLKVQKAY |
| | HLDQSFIDTYIDLLETRRTYYEGPGEGSPFGWKDIKEWYEMLMGHC |
| | TYFPEELRSVKYAYNADLYNALNDLNNLVITRDENEKLEYYEKFQIIE |
| | NVFKQKKKPTLKQIAKEILVNEEDIKGYRVTSTGKPEFTNLKVYHDIK |
| | DITARKEIIENAELLDQIAKILTIYQSSEDIQEELTNLNSELTQEEIEQISN |
| | LKGYTGTHNLSLKAINLILDELWHTNDNQIAIFNRLKLVPKKVDLSQQ |
| | KEIPTTLVDDFILSPVVKRSFIQSIKVINAIIKKYGLPNDIIIELAREKNSK |
| | DAQKMINEMQKRNRQTNERIEEIIRTTGKENAKYLIEKIKLHDMQEGK |
| | CLYSLEAIPLEDLLNNPFNYEVDHIIPRSVSFDNSFNNKVLVKQEENSK |
| | KGNRTPFQYLSSSDSKISYETFKKHILNLAKGKGRISKTKKEYLLEERD |
| | INRFSVQKDFINRNLVDTRYATRGLMNLLRSYFRVNNLDVKVKSING |
| | GFTSFLRRKWKFKKERNKGYKHHAEDALIIANADFIFKEWKKLDKAK |
| | KVMENQMFEEKQAESMPEIETEQEYKEIFITPHQIKHIKDFKDYKYSH |
| | RVDKKPNRELINDTLYSTRKDDKGNTLIVNNLNGLYDKDNDKLKKLI |
| | NKSPEKLLMYHHDPQTYQKLKLIMEQYGDEKNPLYKYYEETGNYLT |
| | KYSKKDNGPVIKKIKYYGNKLNAHLDITDDYPNSRNKVVKLSLKPYR |
| | FDVYLDNGVYKFVTVKNLDVIKKENYYEVNSKCYEEAKKLKKISNQ |
| | AEFIASFYNNDLIKINGELYRVIGVNNDLLNRIEVNMIDITYREYLENM |
| | NDKRPPRIIKTIASKTQSIKKYSTDILGNLYEVKSKKHPQIIKKG (SEQ |
| | ID NO: 342) |

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus, for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

1. (canceled)

2. (canceled)

3. A deaminase comprising an amino acid sequence that is at least 80%, 85%, 90%, 95%, 98%, 99%, or 99.5% identical to the amino acid sequence of SEQ ID NO: 41 wherein the amino acid corresponding to residue 28 of SEQ ID NO: 41 is any amino acid except for V.

4-25. (canceled)

26. The deaminase of claim 3, comprising the mutations E27K, V28A, M61I, and H96N.

27-34. (canceled)

35. A deaminase that comprises mutations at residues R26, V28, A48, and Y73 in the amino acid sequence of SEQ ID NO: 41, or corresponding mutations in a homologous adenosine deaminase (e.g., TadA-dual, SEQ ID NO: 39).

36. (canceled)

37. (canceled)

38. The deaminase of any one of claim 35, comprising the mutations R26G, V28A, A48R, Y73S, and H96N.

39. (canceled)

40. (canceled)

41. A deaminase comprising an amino acid sequence that is at least 80%, 85%, 90%, 95%, 98%, 99%, or 99.5% identical to the amino acid sequence of SEQ ID NO: 39, wherein the amino acid corresponding to residue 46 of SEQ ID NO: 39 is any amino acid except for N.

42-53. (canceled)

54. The deaminase of claim 41, wherein the deaminase comprises a N46C mutation and further comprises a mutation at residue S73P in the amino acid sequence of SEQ ID NO: 39, or a corresponding mutation in a homologous adenosine deaminase.

55. (canceled)

56. (canceled)

57. The deaminase of claim 41, wherein the deaminase comprises a N46V mutation and further comprises a mutation at residue S73P in the amino acid sequence of SEQ ID NO: 39, or a corresponding mutation in a homologous adenosine deaminase.

58. (canceled)

59. The deaminase of claim 41, wherein the deaminase comprises a N46V mutation and further comprises a mutation at residue S73P in the amino acid sequence of SEQ ID NO: 39, or a corresponding mutation in a homologous adenosine deaminase.

60. (canceled)

61. (canceled)

62. The deaminase of claim 41, wherein the deaminase comprises a N46C mutation and further comprises a mutation at residue S73P in the amino acid sequence of SEQ ID NO: 39, or a corresponding mutation in a homologous adenosine deaminase.

63. (canceled)

64. The deaminase of claim 41, wherein the deaminase comprises a N46L mutation and further comprises a mutation at residue S73P in the amino acid sequence of SEQ ID NO: 39, or a corresponding mutation in a homologous adenosine deaminase.

65-85. (canceled)

86. A base editor comprising a nucleic acid programmable DNA binding protein (napDNAbp) domain and a TadA-CD domain comprising the deaminase of claim 41.

87-107. (canceled)

108. A complex comprising the base editor of claim 86 and a guide RNA bound to the napDNAbp domain of the base editor.

109-123. (canceled)

124. A polynucleotide encoding the base editor of claim 86.

125. (canceled)

126. (canceled)

127. A vector comprising the polynucleotide of claim 124.

128-132. (canceled)

133. A recombinant adeno-associated viral (rAAV) particle comprising the AAV vector of claim 127.

134. A cell comprising the rAAV particle of claim 133.

135-138. (canceled)

139. A pharmaceutical composition comprising the base editor of claim 86, the complex of claim 108, or the vector of claim 127.

140. (canceled)

141. A method comprising contacting a nucleic acid with the base editor of claim 86, or the complex of claim 108.

142-183. (canceled)

184. A method comprising administering to a subject the vector of claim 127, the cell of claim 134, or the pharmaceutical composition of claim 139.

185-187. (canceled)

188. A kit comprising a nucleic acid construct, comprising:
(a) a nucleic acid sequence encoding the base editor of claim 86;
(b) a nucleic acid sequence encoding a gRNA; and
(c) one or more heterologous promoters that drive the expression of the sequence of (a) and/or the sequence of (b).

189-201. (canceled)


SEQUENCE LISTING (386 sequences)

SEQ ID NO: 1 - protein, 166 aa, Synthetic construct
SEVEFSHEYWMRHALTLAKRARDEGEVPVGAVLVLNNRVIGEGWNRAIGLYDPTAHAEIMALRQGGLVMQNYRLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 2 - protein, 166 aa, Synthetic construct
SEVEFSHEYWMRHALTLAKRARDEGEVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 3 - protein, 166 aa, Synthetic construct
SEVEFSHEYWMRHALTLAKRARDEREVPVGAVLVLNNRVIGEGWNRAIGLHDPTAHAEIMALRQGGLVMQNYGLIDATLYVTFEPCVMCAGAIIHSRIGRVVFGVRNSKRGAAGSLMNVLNYPGMNHRVEITEGILADECAALLCDFYRMPRQVFNAQKKAQSSIN

SEQ ID NO: 4 - protein, 166 aa, Synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 5 - protein, 166 aa, Synthetic construct
SEVEFSHEYWMRHALTLAKRARDEGEVPVGAVLVLNNRVIGEGWNRAIGLYDPTAHAEIMALRQGGLVMQNYGLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 6 - protein, 166 aa, Synthetic construct
SEVEFSHEYWMRHALTLAKRARDEGEVPVGAVLVLNNRVIGEGWNRAIGLYDPTAHAEIMALRQGGLVMQNYGLIDATLYSTFEPCVMCAGAMIHSRIGRVVFGVRNSKRGAAGSLMNVLNYPGMDHRVEITEGILADECAALLCDFYRMPRRVFNAQKKAQSSIN

SEQ ID NO: 7 - DNA, 182 bp, Synthetic construct
  source 1..182: mol_type = other DNA
cattgcagagaggcgtatcatttcgcggatgttccaatcagtacgcagagagtcgccgtctccaaggtgaaagcggaagtagggccttcgcgcacctcatggaatcccttctgcagcacctggatcgcttttccgagcttctggcggtctcaagcactacctacgtcagcacctgggacccc

SEQ ID NO: 8 - DNA, 277 bp, Synthetic construct
  source 1..277: mol_type = other DNA
cagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgag

SEQ ID NO: 9 - DNA, 219 bp, Synthetic construct
  source 1..219: mol_type = other DNA
gccagaaaagagatatggcatctactcttagacataacacaccagggtcaatacaactttgaagctagtctagtgcaagctaacagttgcttttatcacaggctccaggaagggtttggcctctgattagggtgggggcgtgggtggggtagaagaggactggcagacctctccatcggtggccgtttgcccaggggggcctctttcggaaggctctct

SEQ ID NO: 10 - DNA, 297 bp, Synthetic construct
  source 1..297: mol_type = other DNA
atgtgggctgcctagaaaggcatggatgagagaagcctggagacagggatcccagggaaacgcccatgcaattagtctatttctgctgcaagtaagcatgcatttgtaggcttgatgctttttttctgcttctccagccctggcctgggtcaatccttggggcccagactgagcacgtgatggcagaggaaaggaagccctgcttcctccagagggcgtcgcaggacagcttttcctagacaggggctagtatgtgcagctcctgcaccgggatactggttgacaagtttggctggg

SEQ ID NO: 11 - DNA, 206 bp, Synthetic construct
  source 1..206: mol_type = other DNA
ggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagt

SEQ ID NO: 12 - DNA, 218 bp, Synthetic construct
  source 1..218: mol_type = other DNA
acgtctcatatgccccttggcagtcatcttagtcattacctgaggtgttcgttgtaactcatataaactgagttcccatgttttgcttaatggttgagttccgtttgtctgcacagcctgagacattgctggaaataaagaagagagaaaaacaattttagtatttggaagggaagtgctatggtctgaatgtatgtgtcccaccaaaattcctacgt

SEQ ID NO: 13 - DNA, 288 bp, Synthetic construct
  source 1..288: mol_type = other DNA
gaacccaggtagccagagacccgctggtcttctttcccctcccctgccctcccctcccttcaagatggctgacaaaggccgggctgggtggaaggaagggaggaagggcgaggcagagggtccaaagcaggatgacaggcaggggcaccgcggcgccccggtggcactgcggctggaggtgggggttaaagcggagactctggtgctgtgtgactacagtgggggccctgccctctctgagcccccgcctccaggcctgtgtgtgtgtctccgttcgggttgaaagga

SEQ ID NO: 14 - DNA, 186 bp, Synthetic construct
  source 1..186: mol_type = other DNA
gacctgccagggactgagggtggaaggtccctccagacccctggctctgggacacctgaccgccgaccccacctacctaagaaccatcctggccgccagcccagttgtagcaccgcccagacgactggccagggcgcctgtgggatctgcatgcctggagcagccccaccagagtgccgccttctc

SEQ ID NO: 15 - DNA, 183 bp, Synthetic construct
  source 1..183: mol_type = other DNA
tctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcg

SEQ ID NO: 16 - DNA, 344 bp, Synthetic construct
  source 1..344: mol_type = other DNA
gctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaa

SEQ ID NO: 17 - DNA, 256 bp, Synthetic construct
  source 1..256: mol_type = other DNA
aagccaggatgaggatgactgtggtcttgagggccttgcgcttctggtggcccttggagtgtgacagcttggagatgataatgcaatagcaggacaggatgacaataccaggcaggataaggccaaccatgatgtgctgaaactggaacacaaccacccacaagtcattggggtagaagcggtcacagatatatctgtcatctgcctcactgacgttggcaaagatgaagtcgggaatagtcagcaggagggcagg

SEQ ID NO: 18 - DNA, 261 bp, Synthetic construct
  source 1..261: mol_type = other DNA
gcgctcgacaccgatatcataaacattctgtttgcctaaatatttacgcgtggcaattttgattgagccaccttctgccctggtccaggcgaacgcgaagtcggatcctccggaattcgggagattatccacgcgcatcaggtctaattcgcgttcaaaaatctcatcaatggggagcatctgaccatctacagtcataaatttgtgatcctttgtggcacggattaaagatccgtcttccagacaatattcgaacacttc

SEQ ID NO: 19 - protein, 1699 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRQVFNSQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 20 - protein, 1699 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRRVFNSQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 21 - protein, 1699 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRIPRQVFNSQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 22 - protein, 1699 aa, Synthetic construct
MKRTADGSEFESPKKKRKVSSEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 23 - protein, 1699 aa, Synthetic construct
MKRTADGSEFESPKKKRKVSSEVEFSHEYWMRHALTLAKRARDERAGPVGAVLVLNNRVIGEGWNRAIGLHDPTAHAEIMALRQGGLVMQNHRLIDATLYVTFEPCVMCAGAMINSRIGRVVFGVRNSKRGAAGSLMNVLNYPGMNHRVEITEGILADECAALLCDFYRMPRHVFNSQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 24 - protein, 1699 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGWRNSKRGAAGSLMNVLNYPGMNHRVEITEGILADECAALLCDFYRMPRQVFNSQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 25 - protein, 1699 aa, Synthetic construct
MKRTADGSEFESPKKKRKVSSEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 26 - protein, 1699 aa, Synthetic construct
MKRTADGSEFESPKKKRKVSSEVEFSHEYWMRHALTLAKRARDEGEAPVGAVLVLNNRVIGEGWNRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 27 - protein, 1699 aa, Synthetic construct
MKRTADGSEFESPKKKRKVSSEVEFSHEYWMRHALTLAKRARDEGEGPVGAVLVLNNRVIGEGWNRRIGLHDPTAHAEIMALRQGGLVMQNCKLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 28 - protein, 1505 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRQVFNSQKKAQSSINSGGSSGGSSGSETPGTSESATPESSGGSSGGSAAFKPNPINYILGLAIGIASVGWAMVEIDEEGNPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIAKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPSSKKQRILLQKFDEDGFKECNLNDTRYVNRFLCQFVADHILLTGKGKRRVVASNGQITNLLRGFWRLRKVRAENDRHHALDAVVVACSTVAMQQKITRFVRYKEMNAFDGKTVDKETGKVLYQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 29 - protein, 1478 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRQVFNSQKKAQSSINSGGSSGGSSGSETPGTSESATPESSGGSSGGSGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 30 - protein, 1792 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRQVFNSQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 31 - protein, 1315 aa, Synthetic construct
MKRTADGSEFESPKKKRKVSSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRQVFNSQKKAQSSINSGGSSGGSSGSETPGTSESATPESSGGSSGGSARILAFAIGISSIGWAFSENDELKDCGVRIFTKVENPKTGESLALPRRLARSARKRYARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKKMQEPEFVYYNAFTSSTVSLIVSKHDNKFETLSKNQKILFKNANEKEVIAKSIGIQNLKVFEKYIVSALGEVTKAEFRQREDFKKSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 32 - DNA, 57 bp, Synthetic construct
  source 1..57: mol_type = other DNA
acactctttccctacacgacgctcttccgatctnnnncattgcagagaggcgtatca

SEQ ID NO: 33 - protein, 166 aa, Synthetic construct
SEVEFSHEYWMRHALTLAKRARDEGEVPVGAVLVLNNRVIGEGWNRAIGLYDPTAHAEIMALRQGGLVMQNYRLIDATLYVTFEPCVMCAGAMIHSRIGRVVFGVRNSKRGAAGSLMNVLNYPGMDHRVEITEGILANECAALLCDFYRMPRQVFNAQKKAQSSIN

SEQ ID NO: 34 - protein, 167 aa, Synthetic construct
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRQVFNSQKKAQSSIN

SEQ ID NO: 35 - protein, 167 aa, Synthetic construct
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRRVFNSQKKAQSSIN

SEQ ID NO: 36 - protein, 167 aa, Synthetic construct
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRIPRQVFNSQKKAQSSIN

SEQ ID NO: 37 - protein, 167 aa, Synthetic construct
MSEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 38 - protein, 167 aa, Synthetic construct
MSEVEFSHEYWMRHALTLAKRARDERAGPVGAVLVLNNRVIGEGWNRAIGLHDPTAHAEIMALRQGGLVMQNHRLIDATLYVTFEPCVMCAGAMINSRIGRVVFGVRNSKRGAAGSLMNVLNYPGMNHRVEITEGILADECAALLCDFYRMPRHVFNSQKKAQSSIN

SEQ ID NO: 39 - protein, 167 aa, Synthetic construct
MSEVEFSHEYWMRHALTLAKRARDEGEAPVGAVLVLNNRVIGEGWNRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 40 - protein, 167 aa, Synthetic construct
MSEVEFSHEYWMRHALTLAKRARDEGEGPVGAVLVLNNRVIGEGWNRRIGLHDPTAHAEIMALRQGGLVMQNCRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 41 - protein, 167 aa, Synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 42 - protein, 167 aa, Synthetic construct
MSEVEFSHEYWMRHALTLAKRARDEGEAPVGAVLVLNNRVIGEGWIRRIGLHDPTAHAEIMALRQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 43 - protein, 167 aa, Synthetic construct
MSEVEFSHEYWMRHALTLAKRARDEGEAPVGAVLVLNNRVIGEGWTRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRHVFNAQKKAQSSIN

SEQ ID NO: 44 - protein, 167 aa, Synthetic construct
MSEVEFSHEYWMRHALTLAKRARDEGEAPVGAVLVLNNRVIGEGWTRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 45 - protein, 167 aa, Synthetic construct
MSEVEFSHEYWMRHALTLAKRARDEGEAPVGAVLVLNNRVIGEGWVRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 46 - protein, 167 aa, Synthetic construct
MSEVEFSHEYWMRHALTLAKRARDEGEAPVGAVLVLNNRVIGEGWVRRIGLHDPTAHAEIMALRQGGLVMQNPRLIDATLYVTFEPCVMCAGAMINSRIGRVVFSVRNSKRGAAGSLMNVLNYPGMNHRVEITEGILADECAALLCDFYRMPRQVFNAQKKAQSSIN

SEQ ID NO: 47 - protein, 167 aa, Synthetic construct
MSEVEFSHEYWMRHALTLAKRARDEGEAPVGAVLVLNNRVIGEGWLRRIGLHDPTAHAEIMALRQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 48 - protein, 167 aa, Synthetic construct
MSEVEFSHEYWMRHALTLAKRARDEREAPVGAVLVLNNRVIGEGWLRPIGLHDPTAHAEIMALRQGGLVMQNPRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 49 - protein, 167 aa, Synthetic construct
MSEVEFSHEYWMRHALTLAKRARDEGEAPVGAVLVLNNRVIGEGWCRRIGLHDPTAHAEIMALRQGGLVMQNSRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 50 - protein, 167 aa, Synthetic construct
MSEVEFSHEYWMRHALTLAKRARDEGEAPVGAVLVLNNRVIGEGWVRRIGLHDPTAHAEIMALRQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 51 - protein, 167 aa, Synthetic construct
MSEVEFSHEYWMRHALTLAKRARDEGEAPVGAVLVLNNRVIGEGWVRRIGLHDPTAHAEIMALRQGGLVMHNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 52 - protein, 167 aa, Synthetic construct
MSEVEFSHEYWMRHALTLAKRARDEGEAPVGAVLVLNNRVIGEGWLRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 53 - protein, 167 aa, Synthetic construct
MSEVEFSHEYWMRHALTLAKRARDEGEAPVGAVLVLNNRVIGEGWCRRIGLHDPTAHAEIMALRQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 54 - protein, 167 aa, Synthetic construct
MSEVEFSHEYWMRHALTLAKRARDEGEAPVGAVLVLNNRVIGEGWCRRIGLHDPTAHAEIMALRQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKVQSSIN

SEQ ID NO: 55 - DNA, 57 bp, Synthetic construct
  source 1..57: mol_type = other DNA
acactctttccctacacgacgctcttccgatctnnnncagctcagcctgagtgttga

SEQ ID NO: 56 - DNA, 59 bp, Synthetic construct
  source 1..59: mol_type = other DNA
acactctttccctacacgacgctcttccgatctnnnngccagaaaagagatatggcatc

SEQ ID NO: 57 - DNA, 57 bp, Synthetic construct
  source 1..57: mol_type = other DNA
acactctttccctacacgacgctcttccgatctnnnnatgtgggctgcctagaaagg

SEQ ID NO: 58 - DNA, 58 bp, Synthetic construct
  source 1..58: mol_type = other DNA
acactctttccctacacgacgctcttccgatctnnnnggcgggcattcctgaagctga

SEQ ID NO: 59 - DNA, 57 bp, Synthetic construct
  source 1..57: mol_type = other DNA
acactctttccctacacgacgctcttccgatctnnnnacgtctcatatgccccttgg

SEQ ID NO: 60 - DNA, 57 bp, Synthetic construct
  source 1..57: mol_type = other DNA
acactctttccctacacgacgctcttccgatctnnnngaacccaggtagccagagac

SEQ ID NO: 61 - DNA, 57 bp, Synthetic construct
  source 1..57: mol_type = other DNA
acactctttccctacacgacgctcttccgatctnnnngacctgccagggactgaggg

SEQ ID NO: 62 - DNA, 57 bp, Synthetic construct
  source 1..57: mol_type = other DNA
acactctttccctacacgacgctcttccgatctnnnntctgccttggggaaaaccgt

SEQ ID NO: 63 - DNA, 56 bp, Synthetic construct
  source 1..56: mol_type = other DNA
acactctttccctacacgacgctcttccgatctnnnngctggtcatcctcatcctg

SEQ ID NO: 64 - DNA, 58 bp, Synthetic construct
  source 1..58: mol_type = other DNA
acactctttccctacacgacgctcttccgatctnnnnaagccaggatgaggatgactg

SEQ ID NO: 65 - DNA, 63 bp, Synthetic construct
  source 1..63: mol_type = other DNA
acactctttccctacacgacgctcttccgatctnnnngaagtgttcgaatattgtctggaaga

SEQ ID NO: 66 - DNA, 47 bp, Synthetic construct
  source 1..47: mol_type = other DNA
tggagttcagacgtgtgctcttccgatctggggtcccaggtgctgac

SEQ ID NO: 67 - DNA, 48 bp, Synthetic construct
  source 1..48: mol_type = other DNA
tggagttcagacgtgtgctcttccgatctctcgtgggtttgtggttgc

SEQ ID NO: 68 - DNA, 49 bp, Synthetic construct
  source 1..49: mol_type = other DNA
tggagttcagacgtgtgctcttccgatctagagagccttccgaaagagg

SEQ ID NO: 69 - DNA, 49 bp, Synthetic construct
  source 1..49: mol_type = other DNA
tggagttcagacgtgtgctcttccgatctcccagccaaacttgtcaacc

SEQ ID NO: 70 - DNA, 52 bp, Synthetic construct
  source 1..52: mol_type = other DNA
tggagttcagacgtgtgctcttccgatctacttggagaagggaagtcacgga

SEQ ID NO: 71 - DNA, 51 bp, Synthetic construct
  source 1..51: mol_type = other DNA
tggagttcagacgtgtgctcttccgatctacgtaggaattttggtgggaca

SEQ ID NO: 72 - DNA, 49 bp, Synthetic construct
  source 1..49: mol_type = other DNA
tggagttcagacgtgtgctcttccgatcttcctttcaacccgaacggag

SEQ ID NO: 73 - DNA, 49 bp, Synthetic construct
  source 1..49: mol_type = other DNA
tggagttcagacgtgtgctcttccgatctgagaaggcggcactctggtg

SEQ ID NO: 74 - protein, 1367 aa, Synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 75 - protein, 1367 aa, Synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 76 - protein, 1367 aa, Synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGD

SEQ ID NO: 77 - protein, 1368 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 78 - RNA, 91 nt, Synthetic construct
  source 1..91: mol_type = other RNA
gttttagtactctgtaatgaaaattacagaatctactaaaacaaggcaaaatgccgtgtttatctcgtcaacttgttggcgagattttttt

SEQ ID NO: 79 - DNA, 81 bp, Synthetic construct
  source 1..81: mol_type = other DNA
gttttagagctagaaatagcaagttaaaataaggctagtccgttatcaacttgaaaaagtggcaccgagtcggtgcttttt

SEQ ID NO: 80 - DNA, 49 bp, Synthetic construct
  source 1..49: mol_type = other DNA
tggagttcagacgtgtgctcttccgatctttcctgggagagacgcaaac

SEQ ID NO: 81 - DNA, 49 bp, Synthetic construct
  source 1..49: mol_type = other DNA
tggagttcagacgtgtgctcttccgatctcctgccctcctgctgactat

SEQ ID NO: 82 - DNA, 47 bp, Synthetic construct
  source 1..47: mol_type = other DNA
tggagttcagacgtgtgctcttccgatctgcgctcgacaccgatatc

SEQ ID NO: 83 - DNA, 212 bp, Synthetic construct
  source 1..212: mol_type = other DNA
gctcagaaaaagggccctgacaactcttttcatcttctaggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctc

SEQ ID NO: 84 - DNA, 210 bp, Synthetic construct
  source 1..210: mol_type = other DNA
cattccctctttagccagagccggggtgtgcagacggcagtcactagggggcgctcggccaccacagggaagctgggtgaatggagcgagcagcgtcttcgagagtgaggacgtgtgtgtctgtgtgggtgagtgagtgtgtgcgtgtggggttgagggcgttggagcggggagaaggccaggggtcactccaggattccaatagatctg

SEQ ID NO: 85 - DNA, 208 bp, Synthetic construct
  source 1..208: mol_type = other DNA
ggcgaggcagagggtccaaagcaggatgacaggcaggggcaccgcggcgccccggtggcactgcggctggaggtgggggttaaagcggagactctggtgctgtgtgactacagtgggggccctgccctctctgagcccccgcctccaggcctgtgtgtgtgtctccgttcgggttgaaaggagcccgggaaaaaggccccagaaggag

SEQ ID NO: 86 - DNA, 232 bp, Synthetic construct
  source 1..232: mol_type = other DNA
ctacctgcgccacatccatcggcgctttggtcggcatggccccattcgcacggctctggagcggcggctgcacaaccagtggaggcaagagggcggctttgggcggggtccagttccgggattagcgaacttccaggccctcggtcactgtgacgtcctgctctctctgcgcctgctggagaaccgggccctcggggatgcagctcgttaccacctggtgcagcaactcttt

SEQ ID NO: 87 - DNA, 57 bp, Synthetic construct
  source 1..57: mol_type = other DNA
acactctttccctacacgacgctcttccgatctnnnngctcagaaaaagggccctga

SEQ ID NO: 88 - DNA, 61 bp, Synthetic construct
  source 1..61: mol_type = other DNA
acactctttccctacacgacgctcttccgatctnnnncattccctctttagccagagccgg

SEQ ID NO: 89 - DNA, 57 bp, Synthetic construct
  source 1..57: mol_type = other DNA
acactctttccctacacgacgctcttccgatctnnnnggcgaggcagagggtccaaa

SEQ ID NO: 90 - DNA, 57 bp, Synthetic construct
  source 1..57: mol_type = other DNA
acactctttccctacacgacgctcttccgatctnnnnaaagagttgctgcaccaggt

SEQ ID NO: 91 - DNA, 49 bp, Synthetic construct
  source 1..49: mol_type = other DNA
tggagttcagacgtgtgctcttccgatctgagattcagtgtggtggggg

SEQ ID NO: 92 - DNA, 56 bp, Synthetic construct
  source 1..56: mol_type = other DNA
tggagttcagacgtgtgctcttccgatctcagatctattggaatcctggagtgacc

SEQ ID NO: 93 - DNA, 51 bp, Synthetic construct
  source 1..51: mol_type = other DNA
tggagttcagacgtgtgctcttccgatctctccttctggggcctttttccc

SEQ ID NO: 94 - DNA, 49 bp, Synthetic construct
  source 1..49: mol_type = other DNA
tggagttcagacgtgtgctcttccgatctctacctgcgccacatccatc

SEQ ID NO: 95 - DNA, 175 bp, Synthetic construct
  source 1..175: mol_type = other DNA
tcccctgttgacctggagaagcatgaaccagtcaaaaagtttaaagacaagagcattaactgcaccagtgggcagctcagctcagacaccagtagcgtgggcacccagactgagcacgtgctggagcccaagaaatgcagagacctgtgcacctctggtcagggcaagtacagtg

SEQ ID NO: 96 - DNA, 175 bp, Synthetic construct
  source 1..175: mol_type = other DNA
ttggtgttgacagggagcaacttcacagtcccaggcatcaggacacagactgggcacgtgagggaagcccaagggagaggactggtgtaatcgaggctgactccacttttaatgtttgactgatgataggtttcaagtctcactaagtctccttccccttctgcccacatctcag

SEQ ID NO: 97 - DNA, 175 bp, Synthetic construct
  source 1..175: mol_type = other DNA
tgagagggaacagaagggctaagactaaaaggaacagaggagttcatagtgagcggtaaagagctcagactgagcaagtgaggggctcagcctcccatggaggacagggggctggggcccctggctgatgtctggactgaagcccccacgcccagaggttcttgggcctttggac

SEQ ID NO: 98 - DNA, 174 bp, Synthetic construct
  source 1..174: mol_type = other DNA
cctagcactttggaaggtcgaagcggcaggatggcttcaacccaggagttcgagaccagactgagcaagagagggagagtgtctgtattaacaacaaacaaacaaacaaaaaactaaactaaaagaaactgtggtgtataatataaaattctggctgagcagattaagatgagc

SEQ ID NO: 99 - DNA, 175 bp, Synthetic construct
  source 1..175: mol_type = other DNA
aaaggagcagctcttcctggtggaaattgcgagcagaggctgcgtgagttccgtaactcgcacacagcctccatttggagccagaatgagcacgtgagggaccccgggcagaggggccagtgctgacattatgctccatgcaacctcccatcctgttgtgggagatggtgcagac

SEQ ID NO: 100 - DNA, 7917 bp, Synthetic construct
  source 1..7917: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcagttctgaggtggagttttcccacgagtactggatgagacatgccctgaccctggccaagagggcacgggatgagggggcgggtcctgtgggagccgtgctggtgctgaacaatagagtgatcggcgagggctggaacagagccatcggcctgcacgacccaacagcccatgccgaaattatggccctgagacagggcggcctggtcatgcagaactacagactgtttgacgccaccctgtacgtgacattcgagccttgcgtgatgtgcgccggcgccatgatcaactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaattctcagaagaaggcccagagctccatcaactctggcggatctagcggaggatcctctggcagcgagacaccaggaacaagcgagtcagcaacaccagagagcagtggcggcagcagcggcggcagcgcagcattcaagcctaacccaatcaattacatcctgggactggcaatcggaatcgcatccgtgggatgggctatggtggagatcgacgaggaggggaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcacgagcttgccaaacaccccctggcagctgagagcagccgccctggacaggaagctgacaccactggagtggtctgccgtgctgctgcacctgatcaagcaccgcggctacctgagccagcggaagaacgagggagagacagcagccaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctggggcactgcaccctcgagcctacagagccaaaggccgccaagaacacctacacagccgagcggtttatctggctgacaaagctgaacaatctgagaatcctggagcagggatccgagaggccactgaccgacacagagaggtccaccctgatggatgagccttaccggaagtctaaactgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgttcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgcgaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaagagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccagtagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtggtcgcctctaatggccagatcacaaacctgctgagggggttttggagactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccgtcgataaggagacaggcaaggtgctgtaccagaagacccacttcccccagccttgggagttctttgcccaggaagttatgatccgggtgttcggcaagccagacggcaagcctgagtttgaggaggccgataccccagagaagctgaggacactgctggcagagaagctgtctagcaggccagaggcagtgcacgagtacgtgaccccgctgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccagaaaagcggcgtgctgctgaataagaagaacgcctacacaatcgccgacaatggtgatatggtgagagtggacgtgttctgtaaggtggataagaagggcaagaatcagtactttatcgtgcctatctatgcctggcaggtggccgagaacatcctgccagacatcgattgcaagggctacagaatcgacgatagctatacattctgtttttccctgcacaagtatgacctgatcgccttccagaaggatgagaagtccaaggtggagtttgcctactatatcaattgcgactcctctagcggcgggttctacctggcctggcacgataagggcagcagggagcagcggtttcgcatctccacccagaatctggcgctgatccagaagtatcaggtgaacgagctgggcaaggagatcaggccatgtcggctgaagaagcgcccacccgtgagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgac

SEQ ID NO: 101 - DNA, 7347 bp, Synthetic construct
  source 1..7347: mol_type = other DNA
attgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaaaagatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcagttctgaggtggagttttcccacgagtactggatgagacatgccctgaccctggccaagagggcacgggatgagggggcgggtcctgtgggagccgtgctggtgctgaacaatagagtgatcggcgagggctggaacagagccatcggcctgcacgacccaacagcccatgccgaaattatggccctgagacagggcggcctggtcatgcagaactacagactgtttgacgccaccctgtacgtgacattcgagccttgcgtgatgtgcgccggcgccatgatcaactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaattctcagaagaaggcccagagctccatcaactctggcggatctagcggaggatcctctggcagcgagacaccaggaacaagcgagtcagcaacaccagagagcagtggcggcagcagcggcggcagcgcccgcatcctcgctttcgcaatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggtatgctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgaccatatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgct

SEQ ID NO: 102 - DNA, 8778 bp, Synthetic construct
  source 1..8778: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcagttctgaggtggagttttcccacgagtactggatgagacatgccctgaccctggccaagagggcacgggatgagggggcgggtcctgtgggagccgtgctggtgctgaacaatagagtgatcggcgagggctggaacagagccatcggcctgcacgacccaacagcccatgccgaaattatggccctgagacagggcggcctggtcatgcagaactacagactgtttgacgccaccctgtacgtgacattcgagccttgcgtgatgtgcgccggcgccatgatcaactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaattctcagaagaaggcccagagctccatcaactctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcgggaagaggcggtttgcgtattgggcgcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaaaatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgac

SEQ ID NO: 103 - DNA, 7836 bp, Synthetic construct
  source 1..7836: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcagttctgaggtggagttttcccacgagtactggatgagacatgccctgaccctggccaagagggcacgggatgagggggcgggtcctgtgggagccgtgctggtgctgaacaatagagtgatcggcgagggctggaacagagccatcggcctgcacgacccaacagcccatgccgaaattatggccctgagacagggcggcctggtcatgcagaactacagactgtttgacgccaccctgtacgtgacattcgagccttgcgtgatgtgcgccggcgccatgatcaactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaattctcagaagaaggcccagagctccatcaactctggcggatctagcggaggatcctctggcagcgagacaccaggaacaagcgagtcagcaacaccagagagcagtggcggcagcagcggcggcagcgggaagcgaaattacattctggggctggccattggcattacatcagtgggctatggcatcattgactacgagacaagggacgtgatcgacgccggcgtgagactgttcaaggaggccaacgtggagaacaatgagggccggagatccaagaggggagcaaggcgcctgaagcggagaaggcgccacagaatccagagagtgaagaagctgctgttcgattacaacctgctgaccgaccactccgagctgtctggcatcaatccttatgaggccagagtgaagggcctgtcccagaagctgtctgaggaggagtttagcgccgccctgctgcacctggcaaagaggagaggcgtgcacaacgtgaatgaggtggaggaggacaccggcaacgagctgtccacaaaggagcagatcagccgcaattccaaggccctggaggagaagtatgtggccgagctgcagctggagcggctgaagaaggatggcgaggtgaggggctccatcaatcgcttcaagacctctgactacgtgaaggaggccaagcagctgctgaaggtgcagaaggcctaccaccagctggatcagtcctttatcgatacatatatcgacctgctggagacaaggcgcacatactatgagggaccaggagagggctctcccttcggctggaaggacatcaaggagtggtacgagatgctgatgggccactgcacctattttccagaggagctgagaagcgtgaagtacgcctataacgccgatctgtacaacgccctgaatgacctgaacaacctggtcatcaccagggatgagaacgagaagctggagtactatgagaagttccagatcatcgagaacgtgttcaagcagaagaagaagcctacactgaagcagatcgccaaggagatcctggtgaacgaggaggacatcaagggctaccgcgtgacctccacaggcaagccagagttcaccaatctgaaggtgtatcacgatatcaaggacatcacagcccggaaggagatcatcgagaacgccgagctgctggatcagatcgccaagatcctgaccatctatcagagctccgaggacatccaggaggagctgaccaacctgaatagcgagctgacacaggaggagatcgagcagatcagcaatctgaagggctacaccggcacacacaacctgagcctgaaggccatcaatctgatcctggatgagctgtggcacacaaacgacaatcagatcgccatctttaaccggctgaagctggtgccaaagaaggtggacctgtcccagcagaaggagatcccaaccacactggtggacgatttcatcctgtctcccgtggtgaagcggagcttcatccagagcatcaaagtgatcaacgccatcatcaagaagtacggcctgcccaatgatatcatcatcgagctggccagggagaagaactccaaggacgcccagaagatgatcaatgagatgcagaagaggaaccgccagaccaatgagcggatcgaggagatcatcagaaccacaggcaaggagaacgccaagtacctgatcgagaagatcaagctgcacgatatgcaggagggcaagtgtctgtattctctggaggccatccctctggaggacctgctgaacaatccattcaactacgaggtggatcacatcatcccccggagcgtgagcttcgacaattcttttaacaataaggtgctggtgaagcaggaggagaacagcaagaagggcaataggacccctttccagtacctgtctagctccgattctaagatcagctacgagacattcaagaagcacatcctgaatctggccaagggcaagggccgcatcagcaagaccaagaaggagtacctgctggaggagcgggacatcaacagattctccgtgcagaaggacttcatcaaccggaatctggtggacaccagatacgccacacgcggcctgatgaatctgctgcggtcttatttcagagtgaacaatctggatgtgaaggtaaagagcatcaacggcggcttcacctcctttctgcggagaaagtggaagtttaagaaggagcgcaacaagggctataagcaccacgccgaggatgccctgatcatcgccaatgccgacttcatctttaaggagtggaagaagctggacaaggccaagaaagtgatggagaaccagatgttcgaggagaagcaggccgagagcatgcccgagatcgagacagagcaggagtacaaggagattttcatcacacctcaccagatcaagcacatcaaggacttcaaggactacaagtattctcacagggtggataagaagcccaaccgcgagctgatcaatgacaccctgtatagcacacggaaggacgataagggcaataccctgatcgtgaacaatctgaacggcctgtacgacaaggataatgacaagctgaagaagctgatcaacaagtctcccgagaagctgctgatgtaccaccacgatcctcagacatatcagaagctgaagctgatcatggagcagtacggcgacgagaagaacccactgtataagtactatgaggagacaggcaactacctgacaaagtatagcaagaaggataatggccccgtgatcaagaagatcaagtactatggcaacaagctgaatgcccacctggacatcaccgacgattaccctaactctcgcaataaggtggtgaagctgagcctgaagccataccggttcgacgtgtacctggacaacggcgtgtataagtttgtgacagtgaagaatctggatgtgatcaagaaggagaactactatgaggtgaacagcaagtgctacgaggaggccaagaagctgaagaagatcagcaaccaggccgagttcatcgcctctttttacaacaatgacctgatcaagatcaatggcgagctgtatagagtgatcggcgtgaacaatgatctgctgaacagaatcgaagtgaatatgatcgacatcacctacagggagtatctggagaacatgaatgataagaggccccctcgcatcatcaagaccatcgcctctaagacacagagcatcaagaagtacagcacagacatcctggggaacctgtatgaagtcaagagcaagaaacatcctcagattatcaagaaaggcagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcgggaagaggcggtttgcgtattgggcgcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaaaatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgac

SEQ ID NO: 104 - DNA, 8778 bp, Synthetic construct
  source 1..8778: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcagttctgaggtggagttttcccacgagtactggatgagacatgccctgaccctggccaagagggcacgggatgagggggcgggtcctgtgggagccgtgctggtgctgaacaatagagtgatcggcgagggctggaacagagccatcggcctgcacgacccaacagcccatgccgaaattatggccctgagacagggcggcctggtcatgcagaactacagactgtttgacgccaccctgtacgtgacattcgagccttgcgtgatgtgcgccggcgccatgatcaactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaattctcagaagaaggcccagagctccatcaactctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccggttcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgac

SEQ ID NO: 105 - DNA, 175 bp, Synthetic construct
  source 1..175: mol_type = other DNA
ggcatggcttctgagactcatagctggggctgaagatccctaggggggctctgctgggctcactgctctccagagtggtccagcccggctgcagggtgctgcttccagcttggtgcactgcggccggaggaggtggaggatggaaagtaagattcaaagacagggagtgcaaggg

SEQ ID NO: 106 - DNA, 175 bp, Synthetic construct
  source 1..175: mol_type = other DNA
tttggcaatggaggcattgggcaggggaagcctgtcttcagggcacatgcacgtgcgcagggctctgcggctggagggggtggggttgctgttagtgacaggggccccagccaggcaggtttcaggattggggagcacttgcttcggctcccttgctctcatgggcagcctcttc

SEQ ID NO: 107 - DNA, 175 bp, Synthetic construct
  source 1..175: mol_type = other DNA
ggtctgaggctcgaatcctggcagcaggtccttcatggcaaggcgggaaaagagaaaagccaacgggttctcatgctgggaaaagatgccgggcacgacggctggaggtggggggttgggagtgggtgggatgcttgcgtgccctgcatgaggtgcagggatatggaggccacag

SEQ ID NO: 108 - DNA, 174 bp, Synthetic construct
  source 1..174: mol_type = other DNA
ttccaccagaactcagcccaggctgctgtgggatggaatcacctgcacccggatgttctttctgggctggtacatacaggcaaggcatcacggctggaggtggagggggcctaacccggggttgcccaggaaggggtttgcacatggattcggtgtgttgtggaggaaccgagg

SEQ ID NO: 109 - DNA, 175 bp, Synthetic construct
  source 1..175: mol_type = other DNA
cacgggaaggacaggagaaggtgctggaccgcctggactttgtgctgaccagccttgtggcgctgcggcgggaggtggaggagctgagaagcagcctgcgagggcttgcgggggagattgttggggaggtccggtgagtaatgcggcttcttctcctgctttatccctcccctgc

SEQ ID NO: 110 - DNA, 185 bp, Synthetic construct
  source 1..185: mol_type = other DNA
tgcccaatcattgatgcttttataccatcttggggttacagaaagaataggggcttatggcatggcaagacagattgtcagagttagagcagaagaagaaaggcatggagtaaaggcaatcttgtgcagatgtacaggtagcagccctcagaaaaaataggtgatagtctatggtaaatgtttct

SEQ ID NO: 111 - DNA, 174 bp, Synthetic construct
  source 1..174: mol_type = other DNA
gtagcctctttctcaatgtgcttcaacccatcacggcctttgcaaatagagccctttattcatagtagacaagagtctaagcagaagaagaagagagccactacccaaccatctactcttctaatggtgttttcctacaaaggccaagtcatgagactgcatccttgtgaaagc

SEQ ID NO: 112 - DNA, 175 bp, Synthetic construct
  source 1..175: mol_type = other DNA
gagctagactccgaggggaggctgcgagccgcaagcgcaggagccgggtgggagagagaccccttcttctgcaaatgaggaggccgagcagaagaaagacggcgacagatgttggggggaggggacggtttgtgagggatagggagagaaagtctaagtgagagcaggacgagga

SEQ ID NO: 113 - DNA, 175 bp, Synthetic construct
  source 1..175: mol_type = other DNA
agaggctgaagaggaagaccagactcagtaaagcctggaggctgccaggtagggctggggccagcatgacctgagtcctagcaggagaagaagaggcagcctagagtcttctgtgaagtgcacatagaagagagactggggccaagccacaaaagatagaatgcacagctgggcc

SEQ ID NO: 114 - DNA, 199 bp, Synthetic construct
  source 1..199: mol_type = other DNA
gtagttctgacattcctcctgagggaaaataaataaattaattaaaaatatatatatatatgtataatgataaacatgctaacaaagtctgagcacaagaagaatggtgagaaggaatacattttatctaataaatatgtaagccattaataaaatgtaaaccattaaaacaacaaataaacctttcagatattgacca

SEQ ID NO: 115 - DNA, 176 bp, Synthetic construct
  source 1..176: mol_type = other DNA
ccaagagggccaagtcctggctgtctgcctctgacgacgagcaaggtggaggcccttggttagcaggatgggtggtgaggagtccgggaaggagaagaaaggctcagcgcggcttgcctgagcctccctcctcccagctcccggcccctgctgccggcggctgtcactcctcgctg

SEQ ID NO: 116 - DNA, 175 bp, Synthetic construct
  source 1..175: mol_type = other DNA
cactccacctgatctcggggcgctgtgcgctgaggaaggcgcgggcgagccggagcagaagaaggagggagggagccagccgctgcagccaccaccgccaccatgtcctaccaaggcaagaagaacatcccgcggatcacggtgagtccgggcgccgctgctccctccctcctcg

SEQ ID NO: 117 - DNA, 177 bp, Synthetic construct
  source 1..177: mol_type = other DNA
accacaaatgcccaagagacatcaccacttggagagtcagaggtcacaaaagaggggcccaactcctgtagaagtccgaggagaggaagaaagggttctggagctctcaggcgtcagggccaggcctgcacccttctgtgcccctccatgaatggctggccggcccttgactgtgtc

SEQ ID NO: 118 - DNA, 175 bp, Synthetic construct
  source 1..175: mol_type = other DNA
cccacctttgaggaggcaaaagggaataaacttgtgcttatttgttggaagagcaaatatgtttttttgaaaccgaattatggatggggatgtgggggtgggaactaggcaagggtctcaggggaatccaagcaggagaagaaggagggaaaaaccactctcttctcagatggaa

SEQ ID NO: 119 - DNA, 175 bp, Synthetic construct
  source 1..175: mol_type = other DNA
gtcataccttggcccttcctctgtactctatacagagtccagctctggcctgggaaaatactttcagacaaaacgtctgagcagaagaagaatggacagaactctgaggacattcttgaggcactggcagaacctctgcaggaagacgagagcattgctggtgtgggcctaggga

SEQ ID NO: 120 - DNA, 212 bp, Synthetic construct
  source 1..212: mol_type = other DNA
acctgtgggcatcctgagttgcttctgatgtcccacccatcaccttgacctgctcagagcagagcattgttctgaaatctgaggcattgtcctgcccactggcctatcactggctccaggaagggcctagtgtctctgaccagctctagatcacctcctcctcctcctgagccctgtacgttgccaggctgatgagaggagtggggccgtga

SEQ ID NO: 121 - DNA, 197 bp, Synthetic construct
  source 1..197: mol_type = other DNA
cttggcgcagttcctgtgtatggatattcttacagaatcgctactctccctctcctttgagctggcctagctttggcttatcataggccccaggaaaggccaggggactggggtaccggttagagggatataaaagttcattctgccttgtacgtatgtttaattgattagaacacttcattttctcacagcatgta

SEQ ID NO: 122 - DNA, 225 bp, Synthetic construct
  source 1..225: mol_type = other DNA
gacttctggttatcagtaggctgttagtagttaagttttgggggagtcaaaagttatacctgcatttgactgtgtcaagagttgacaccctaacctccacgttgtttaagagttaactgtagtttgaactacactagtttgtactggtttgcagttatttattatactatattcatcattacttttatgtttcaatgaaccattgttgagccatgatccattttg

SEQ ID NO: 123 - DNA, 236 bp, Synthetic construct
  source 1..236: mol_type = other DNA
tgaggacttggtgaatcgtggtatcatttgtcaagagttgacagataagaggaagggagtaggtttgtcagggaaagggttaattttttttttttggagacttgttgcccaggctggagtacaatggcatgatcttggctcaccacaacttccgcctcctgggttcaagtgattttgctgcctcagcctcccgagtagctgggattacaggcatgtgccaccacacccagctaatt

SEQ ID NO: 124 - DNA, 268 bp, Synthetic construct
  source 1..268: mol_type = other DNA
caattacagaaagtggattggttgtggaatgtacagattaacaatggagagcacataaccctcagtctggcacaccagtggaggctaaagaaactgaaaggtgttctgcccagggtgcagggactccatgtcaagagttggcaggtatggaaagcaagagggttgggacagaaaccagggtaattaattaagtgactatttttaggactaacttcaagggttacatgagctccctcccctccaaaaacaaatcattaggaaaatctca

SEQ ID NO: 125 - DNA, 280 bp, Synthetic construct
  source 1..280: mol_type = other DNA
ttgctgaaagtgacgacacagccagtgaagctgggactggaatgtcagtttccataactccagcccccttttttaaccgttaggaaacatcctttcacctcaggtcccctcagtctctagtccacaccatgatgtgctgagaacaggaggcatactatcagcattttaaaaggacctcttccttgtttctcctgttctctggccccagagagctcttctgagaccctggggtccagaagggagccctggagaacttagctccaaagacttttcctgagca

SEQ ID NO: 126 - DNA, 57 bp, Synthetic construct
  source 1..57: mol_type = other DNA
acactctttccctacacgacgctcttccgatctnnnntcccctgttgacctggagaa

SEQ ID NO: 127 - DNA, 57 bp, Synthetic construct
  source 1..57: mol_type = other DNA
acactctttccctacacgacgctcttccgatctnnnnttggtgttgacagggagcaa

SEQ ID NO: 128 - DNA, 57 bp, Synthetic construct
  source 1..57: mol_type = other DNA
acactctttccctacacgacgctcttccgatctnnnntgagagggaacagaagggct

SEQ ID NO: 129 - DNA, 58 bp, Synthetic construct
  source 1..58: mol_type = other DNA
acactctttccctacacgacgctcttccgatctnnnntcctagcactttggaaggtcg

SEQ ID NO: 130 - DNA, 57 bp, Synthetic construct
  source 1..57: mol_type = other DNA
acactctttccctacacgacgctcttccgatctnnnnaaaggagcagctcttcctgg

SEQ ID NO: 131 - DNA, 57 bp, Synthetic construct
  source 1..57: mol_type = other DNA
acactctttccctacacgacgctcttccgatctnnnnggcatggcttctgagactca

SEQ ID NO: 132 - DNA, 57 bp, Synthetic construct
  source 1..57: mol_type = other DNA
acactctttccctacacgacgctcttccgatctnnnntttggcaatggaggcattgg

SEQ ID NO: 133 - DNA, 57 bp, Synthetic construct
  source 1..57: mol_type = other DNA
acactctttccctacacgacgctcttccgatctnnnnggtctgaggctcgaatcctg

SEQ ID NO: 134 - DNA, 57 bp, Synthetic construct
  source 1..57: mol_type = other DNA
acactctttccctacacgacgctcttccgatctnnnntttccaccagaactcagccc

SEQ ID NO: 135 - DNA, 57 bp, Synthetic construct
  source 1..57: mol_type = other DNA
acactctttccctacacgacgctcttccgatctnnnncacgggaaggacaggagaag

SEQ ID NO: 136 - DNA, 58 bp, Synthetic construct
  source 1..58: mol_type = other DNA
acactctttccctacacgacgctcttccgatctnnnntgcccaatcattgatgctttt

SEQ ID NO: 137 - DNA, 59 bp, Synthetic construct
  source 1..59: mol_type = other DNA
acactctttccctacacgacgctcttccgatctnnnnagtagcctctttctcaatgtgc

SEQ ID NO: 138 - DNA, 56 bp, Synthetic construct
  source 1..56: mol_type = other DNA
acactctttccctacacgacgctcttccgatctnnnngagctagactccgagggga

SEQ ID NO: 139 - DNA, 58 bp, Synthetic construct
  source 1..58: mol_type = other DNA
acactctttccctacacgacgctcttccgatctnnnnagaggctgaagaggaagacca

SEQ ID NO: 140 - DNA, 60 bp, Synthetic construct
  source 1..60: mol_type = other DNA
acactctttccctacacgacgctcttccgatctnnnngtagttctgacattcctcctgag

SEQ ID NO: 141 - DNA, 56 bp, Synthetic construct
  source 1..56: mol_type = other DNA
acactctttccctacacgacgctcttccgatctnnnnccaagagggccaagtcctg

SEQ ID NO: 142 - protein, 7 aa, Synthetic construct
PKKKRKV

SEQ ID NO: 143 - protein, 19 aa, Synthetic construct
MKRTADGSEFESPKKKRKV

SEQ ID NO: 144 - protein, 30 aa, Synthetic construct
MDSLLMNRRKFLYQFKNVRWAKGRRETYLC

SEQ ID NO: 145 - protein, 20 aa, Synthetic construct
AVKRPAATKKAGQAKKKKLD

SEQ ID NO: 146 - protein, 25 aa, Synthetic construct
MSRRRKANPTKLSENAKKLAKEVEN

SEQ ID NO: 147 - protein, 9 aa, Synthetic construct
PAAKRVKLD

SEQ ID NO: 148 - protein, 9 aa, Synthetic construct
KLKIKRPVK

SEQ ID NO: 149 - protein, 8 aa, Synthetic construct
VSRKRPRP

SEQ ID NO: 150 - protein, 10 aa, Synthetic construct
EGAPPAKRAR

SEQ ID NO: 151 - protein, 12 aa, Synthetic construct
PPQPKKKPLDGE

SEQ ID NO: 152 - protein, 21 aa, Synthetic construct
SGGSKRTADGSEFEPKKKRKV

SEQ ID NO: 153 - protein, 18 aa, Synthetic construct
KRTADGSEFESPKKKRKV

SEQ ID NO: 154 - protein, 16 aa, Synthetic construct
KRXXXXXXXXXXKKKL

SEQ ID NO: 155 - protein, 17 aa, Synthetic construct
KRTADGSEFEPKKKRKV

SEQ ID NO: 156 - protein, 150 aa, Synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 157 - protein, 30 aa, Synthetic construct
GGGGGGGGGGGGGGGGGGGGGGGGGGGGGG

SEQ ID NO: 158 - protein, 150 aa, Synthetic construct
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 159 - protein, 90 aa, Synthetic construct
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 160 - protein, 120 aa, Synthetic construct
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 161 - protein, 60 aa, Synthetic construct
XPXPXPXPXPXPXPXPXPXPXPXPXPXPXPXPXPXPXPXPXPXPXPXPXPXPXPXPXPXP

SEQ ID NO: 162 - protein, 16 aa, Synthetic construct
SGSETPGTSESATPES

SEQ ID NO: 163 - protein, 12 aa, Synthetic construct
SGSETPGTSESA

SEQ ID NO: 164 - protein, 21 aa, Synthetic construct
SGSETPGTSESATPEGGSGGS

SEQ ID NO: 165 - protein, 32 aa, Synthetic construct
SGGSSGGSSGSETPGTSESATPESSGGSSGGS

SEQ ID NO: 166 - protein, 10 aa, Synthetic construct
SGGSGGSGGS

SEQ ID NO: 167 - protein, 60 aa, Synthetic construct
SGGSSGGSSGSETPGTSESATPESAGSYPYDVPDYAGSAAPAAKKKKLDGSGSGGSSGGS

SEQ ID NO: 168 - protein, 6 aa, Synthetic construct
GGSGGS

SEQ ID NO: 169 - protein, 9 aa, Synthetic construct
GGSGGSGGS

SEQ ID NO: 170 - protein, 33 aa, Synthetic construct
SGGSSGGSSGSETPGTSESATPESSGGSSGGSS

SEQ ID NO: 171 - DNA, 57 bp, Synthetic construct
  source 1..57: mol_type = other DNA
acactctttccctacacgacgctcttccgatctnnnncactccacctgatctcgggg

SEQ ID NO: 172 - DNA, 57 bp, Synthetic construct
  source 1..57: mol_type = other DNA
acactctttccctacacgacgctcttccgatctnnnnaccacaaatgcccaagagac

SEQ ID NO: 173 - DNA, 57 bp, Synthetic construct
  source 1..57: mol_type = other DNA
acactctttccctacacgacgctcttccgatctnnnncccacctttgaggaggcaaa

SEQ ID NO: 174 - DNA, 57 bp, Synthetic construct
  source 1..57: mol_type = other DNA
acactctttccctacacgacgctcttccgatctnnnngtcataccttggcccttcct

SEQ ID NO: 175 - DNA, 59 bp, Synthetic construct
  source 1..59: mol_type = other DNA
acactctttccctacacgacgctcttccgatctnnnnacctgtgggcatcctgagttgc

SEQ ID NO: 176 - DNA, 59 bp, Synthetic construct
  source 1..59: mol_type = other DNA
acactctttccctacacgacgctcttccgatctnnnncttggcgcagttcctgtgtatg

SEQ ID NO: 177 - DNA, 62 bp, Synthetic construct
  source 1..62: mol_type = other DNA
acactctttccctacacgacgctcttccgatctnnnngacttctggttatcagtaggctgtt

SEQ ID NO: 178 - DNA, 58 bp, Synthetic construct
  source 1..58: mol_type = other DNA
acactctttccctacacgacgctcttccgatctnnnntgaggacttggtgaatcgtgg

SEQ ID NO: 179 - DNA, 61 bp, Synthetic construct
  source 1..61: mol_type = other DNA
acactctttccctacacgacgctcttccgatctnnnncaattacagaaagtggattggttg

SEQ ID NO: 180 - DNA, 58 bp, Synthetic construct
  source 1..58: mol_type = other DNA
acactctttccctacacgacgctcttccgatctnnnnttgctgaaagtgacgacacag

SEQ ID NO: 181 - DNA, 49 bp, Synthetic construct
  source 1..49: mol_type = other DNA
tggagttcagacgtgtgctcttccgatctcactgtacttgccctgacca

SEQ ID NO: 182 - DNA, 49 bp, Synthetic construct
  source 1..49: mol_type = other DNA
tggagttcagacgtgtgctcttccgatctctgagatgtgggcagaaggg

SEQ ID NO: 183 - DNA, 49 bp, Synthetic construct
  source 1..49: mol_type = other DNA
tggagttcagacgtgtgctcttccgatctgtccaaaggcccaagaacct

SEQ ID NO: 184 - DNA, 51 bp, Synthetic construct
  source 1..51: mol_type = other DNA
tggagttcagacgtgtgctcttccgatctgctcatcttaatctgctcagcc

SEQ ID NO: 185 - DNA, 49 bp, Synthetic construct
  source 1..49: mol_type = other DNA
tggagttcagacgtgtgctcttccgatctgtctgcaccatctcccacaa

SEQ ID NO: 186 - DNA, 53 bp, Synthetic construct
  source 1..53: mol_type = other DNA
tggagttcagacgtgtgctcttccgatctgtctcccttgcactccctgtcttt

SEQ ID NO: 187 - DNA, 49 bp, Synthetic construct
  source 1..49: mol_type = other DNA
tggagttcagacgtgtgctcttccgatctgaagaggctgcccatgagag

SEQ ID NO: 188 - DNA, 49 bp, Synthetic construct
  source 1..49: mol_type = other DNA
tggagttcagacgtgtgctcttccgatctctgtggcctccatatccctg

SEQ ID NO: 189 - DNA, 49 bp, Synthetic construct
  source 1..49: mol_type = other DNA
tggagttcagacgtgtgctcttccgatctcctcggttcctccacaacac

SEQ ID NO: 190 - DNA, 49 bp, Synthetic construct
  source 1..49: mol_type = other DNA
tggagttcagacgtgtgctcttccgatctgcaggggagggataaagcag

SEQ ID NO: 191 - DNA, 56 bp, Synthetic construct
  source 1..56: mol_type = other DNA
tggagttcagacgtgtgctcttccgatctagaaacatttaccatagactatcacct

SEQ ID NO: 192 - DNA, 50 bp, Synthetic construct
  source 1..50: mol_type = other DNA
tggagttcagacgtgtgctcttccgatctgctttcacaaggatgcagtct

SEQ ID NO: 193 - DNA, 49 bp, Synthetic construct
  source 1..49: mol_type = other DNA
tggagttcagacgtgtgctcttccgatcttcctcgtcctgctctcactt

SEQ ID NO: 194 - DNA, 49 bp, Synthetic construct
  source 1..49: mol_type = other DNA
tggagttcagacgtgtgctcttccgatctggcccagctgtgcattctat

SEQ ID NO: 195 - DNA, 56 bp, Synthetic construct
  source 1..56: mol_type = other DNA
tggagttcagacgtgtgctcttccgatcttggtcaatatctgaaaggtttatttgt

SEQ ID NO: 196 - DNA, 47 bp, Synthetic construct
  source 1..47: mol_type = other DNA
tggagttcagacgtgtgctcttccgatctcagcgaggagtgacagcc

SEQ ID NO: 197 - DNA, 47 bp, Synthetic construct
  source 1..47: mol_type = other DNA
tggagttcagacgtgtgctcttccgatctcgaggagggagggagcag

SEQ ID NO: 198 - DNA, 47 bp, Synthetic construct
  source 1..47: mol_type = other DNA
tggagttcagacgtgtgctcttccgatctgacacagtcaagggccgg

SEQ ID NO: 199 - DNA, 51 bp, Synthetic construct
  source 1..51: mol_type = other DNA
tggagttcagacgtgtgctcttccgatctttccatctgagaagagagtggt

SEQ ID NO: 200 - protein, 1368 aa, Streptococcus pyogenes [sequence omitted]

SEQ ID NO: 201 - DNA, 47 bp, Synthetic construct
  source 1..47: mol_type = other DNA
tggagttcagacgtgtgctcttccgatcttccctaggcccacaccag

SEQ ID NO: 202 - DNA, 49 bp, Synthetic construct
  source 1..49: mol_type = other DNA
tggagttcagacgtgtgctcttccgatcttcacggccccactcctctca

SEQ ID NO: 203 - DNA, 54 bp, Synthetic construct
  source 1..54: mol_type = other DNA
tggagttcagacgtgtgctcttccgatcttacatgctgtgagaaaatgaagtgt

SEQ ID NO: 204 - protein, 19 aa, Synthetic construct
NLSKRPAAIKKAGQAKKKK

SEQ ID NO: 205 - protein, 11 aa, Synthetic construct
RQRRNELKRSF

SEQ ID NO: 206 - protein, 38 aa, Synthetic construct
NQSSNFGPMKGGNFGGRSSGPYGGGGQYFAKPRNQGGY

SEQ ID NO: 207 - DNA, 49 bp, Synthetic construct
  source 1..49: mol_type = other DNA
tggagttcagacgtgtgctcttccgatctcaaaatggatcatggctcaa

SEQ ID NO: 208 - DNA, 20 bp, Synthetic construct
  source 1..20: mol_type = other DNA
ggaatcccttctgcagcacc

SEQ ID NO: 209 - DNA, 20 bp, Synthetic construct
  source 1..20: mol_type = other DNA
gagtccgagcagaagaagaa

SEQ ID NO: 210 - DNA, 20 bp, Synthetic construct
  source 1..20: mol_type = other DNA
tttatcacaggctccaggaa

SEQ ID NO: 211 - DNA, 20 bp, Synthetic construct
  source 1..20: mol_type = other DNA
ggcccagactgagcacgtga

SEQ ID NO: 212 - DNA, 20 bp, Synthetic construct
  source 1..20: mol_type = other DNA
actcacgctggatagcctcc

SEQ ID NO: 213 - DNA, 20 bp, Synthetic construct
  source 1..20: mol_type = other DNA
gtcatcttagtcattacctg

SEQ ID NO: 214 - DNA, 20 bp, Synthetic construct
  source 1..20: mol_type = other DNA
ggcactgcggctggaggtgg

SEQ ID NO: 215 - DNA, 20 bp, Synthetic construct
  source 1..20: mol_type = other DNA
cacctacctaagaaccatcc

SEQ ID NO: 216 - DNA, 20 bp, Synthetic construct
  source 1..20: mol_type = other DNA
ttcgtatctgtaaaaccaag

SEQ ID NO: 217 - DNA, 20 bp, Synthetic construct
  source 1..20: mol_type = other DNA
caatgtgtcaactcttgaca

SEQ ID NO: 218 - DNA, 20 bp, Synthetic construct
  source 1..20: mol_type = other DNA
cagtttcagcacatcatggt

SEQ ID NO: 219 - DNA, 20 bp, Synthetic construct
  source 1..20: mol_type = other DNA
ggtccaggcgaacgcgaagt

SEQ ID NO: 220 - DNA, 49 bp, Synthetic construct
  source 1..49: mol_type = other DNA
tggagttcagacgtgtgctcttccgatctaattagctgggtgtggtggc

SEQ ID NO: 221 - DNA, 55 bp, Synthetic construct
  source 1..55: mol_type = other DNA
tggagttcagacgtgtgctcttccgatctttgtcttgagattttcctaatgattt

SEQ ID NO: 222 - DNA, 50 bp, Synthetic construct
  source 1..50: mol_type = other DNA
tggagttcagacgtgtgctcttccgatcttgctcaggaaaagtctttgga

SEQ ID NO: 223 - DNA, 270 bp, Synthetic construct
  source 1..270: mol_type = other DNA
tggtggagtgctctgtgtttgtctttataaacccagatgagaggatgaaggcaacaagcttctgtaccaacatacatgcccctttgcctcaagtctggttattttagggggatgctaggttgctttgggtctaccttactgagaaaatggccccaggtcattgtcatgtccagttgtggtagacagcatgtgtcctaaagggtatattcacatgcatgtgcaaaaatacaggggtccttctaaccctatcacagagaagcaggagactgc

SEQ ID NO: 224 - DNA, 255 bp, Synthetic construct
  source 1..255: mol_type = other DNA
gctacagaaaggtcagcagctatatttaacctcagaccagggtgcggtgggagatctggtttccggaagacggaatggggagaagggcaggttccccgaggcgcccagacacccaatcctcccggtgacatttacagcctggcctttggggtcgggtcaacgctaggctggcaggggaagggcggggccgtgaggtgagccggcgctgcaggaaggggccaccaccagaggggccattttgcggtggaaatgtcc

SEQ ID NO: 225 - DNA, 240 bp, Synthetic construct
  source 1..240: mol_type = other DNA
aagtgttcagctgcttttctttcatttattccacatataattactataattgctaaacatttatttagtgtcaggtaatgtgctaaacagagagttactgctcagacatgtaataataataaataacacatcaaataaccataccattttaagctgtagtattatgaagggaaatctggagcaaagagaatagactgtagggaaaccagttaagaaataggacatggaggctaggtgcag

SEQ ID NO: 226 - DNA, 23 bp, Synthetic construct
  source 1..23: mol_type = other DNA
gcaagagcacaagaggaagagag

SEQ ID NO: 227 - DNA, 22 bp, Synthetic construct
  source 1..22: mol_type = other DNA
ggctacagcaacagggtggtgg

SEQ ID NO: 228 - DNA, 23 bp, Synthetic construct
  source 1..23: mol_type = other DNA
ggtgtgcagacggcagtcactag

SEQ ID NO: 229 - DNA, 23 bp, Synthetic construct
  source 1..23: mol_type = other DNA
gtctccgctttaacccccacctc

SEQ ID NO: 230 - DNA, 23 bp, Synthetic construct
  source 1..23: mol_type = other DNA
cctcttgcctccactggttgtgc

SEQ ID NO: 231 - DNA, 23 bp, Synthetic construct
  source 1..23: mol_type = other DNA
ggtccaaagcaggatgacaggca

SEQ ID NO: 232 - DNA, 20 bp, Synthetic construct
  source 1..20: mol_type = other DNA
cacccagactgagcacgtgc

SEQ ID NO: 233 - DNA, 20 bp, Synthetic construct
  source 1..20: mol_type = other DNA
gacacagactgggcacgtga

SEQ ID NO: 234 - DNA, 20 bp, Synthetic construct
  source 1..20: mol_type = other DNA
agctcagactgagcaagtga

SEQ ID NO: 235 - DNA, 20 bp, Synthetic construct
  source 1..20: mol_type = other DNA
agaccagactgagcaagaga

SEQ ID NO: 236 - DNA, 20 bp, Synthetic construct
  source 1..20: mol_type = other DNA
gagccagaatgagcacgtga

SEQ ID NO: 237 - DNA, 20 bp, Synthetic construct
  source 1..20: mol_type = other DNA
tgcactgcggccggaggagg

SEQ ID NO: 238 - DNA, 20 bp, Synthetic construct
  source 1..20: mol_type = other DNA
ggctctgcggctggaggggg

SEQ ID NO: 239 - DNA, 20 bp, Synthetic construct
  source 1..20: mol_type = other DNA
ggcacgacggctggaggtgg

SEQ ID NO: 240 - DNA, 20 bp, Synthetic construct
  source 1..20: mol_type = other DNA
ggcatcacggctggaggtgg

SEQ ID NO: 241 - DNA, 20 bp, Synthetic construct
  source 1..20: mol_type = other DNA
ggcgctgcggcgggaggtgg

SEQ ID NO: 242 - DNA, 20 bp, Synthetic construct
  source 1..20: mol_type = other DNA
gagttagagcagaagaagaa

SEQ ID NO: 243 - DNA, 20 bp, Synthetic construct
  source 1..20: mol_type = other DNA
gagtctaagcagaagaagaa

SEQ ID NO: 244 - DNA, 20 bp, Synthetic construct
  source 1..20: mol_type = other DNA
gaggccgagcagaagaaaga

SEQ ID NO: 245 - DNA, 20 bp, Synthetic construct
  source 1..20: mol_type = other DNA
gagtcctagcaggagaagaa

SEQ ID NO: 246 - DNA, 20 bp, Synthetic construct
  source 1..20: mol_type = other DNA
aagtctgagcacaagaagaa

SEQ ID NO: 247 - DNA, 20 bp, Synthetic construct
  source 1..20: mol_type = other DNA
gagtccgggaaggagaagaa

SEQ ID NO: 248 - DNA, 20 bp, Synthetic construct
  source 1..20: mol_type = other DNA
gagccggagcagaagaagga

SEQ ID NO: 249 - DNA, 20 bp, Synthetic construct
  source 1..20: mol_type = other DNA
aagtccgaggagaggaagaa

SEQ ID NO: 250 - DNA, 20 bp, Synthetic construct
  source 1..20: mol_type = other DNA
gaatccaagcaggagaagaa

SEQ ID NO: 251 - DNA, 20 bp, Synthetic construct
  source 1..20: mol_type = other DNA
acgtctgagcagaagaagaa

SEQ ID NO: 252 - DNA, 20 bp, Synthetic construct
  source 1..20: mol_type = other DNA
cctatcactggctccaggaa

SEQ ID NO: 253 - DNA, 20 bp, Synthetic construct
  source 1..20: mol_type = other DNA
cttatcataggccccaggaa

SEQ ID NO: 254 - DNA, 20 bp, Synthetic construct
  source 1..20: mol_type = other DNA
tagggtgtcaactcttgaca

SEQ ID NO: 255 - DNA, 20 bp, Synthetic construct
  source 1..20: mol_type = other DNA
ttatctgtcaactcttgaca

SEQ ID NO: 256 - DNA, 20 bp, Synthetic construct
  source 1..20: mol_type = other DNA
atacctgccaactcttgaca

SEQ ID NO: 257 - DNA, 20 bp, Synthetic construct
  source 1..20: mol_type = other DNA
tgttctcagcacatcatggt

SEQ ID NO: 258 - DNA, 22 bp, Synthetic construct
  source 1..22: mol_type = other DNA
gtggtagacagcatgtgtccta

SEQ ID NO: 259 - DNA, 22 bp, Synthetic construct
  source 1..22: mol_type = other DNA
atttacagcctggcctttgggg

SEQ ID NO: 260 - DNA, 22 bp, Synthetic construct
  source 1..22: mol_type = other DNA
gtgtcaggtaatgtgctaaaca

SEQ ID NO: 261 - DNA, 22 bp, Synthetic construct
  source 1..22: mol_type = other DNA
ggtggaggagggtgcatggggt

SEQ ID NO: 262 - DNA, 20 bp, Synthetic construct
  source 1..20: mol_type = other DNA
tctgcttctccagccctggc

SEQ ID NO: 263 - DNA, 20 bp, Synthetic construct
  source 1..20: mol_type = other DNA
gatgttccaatcagtacgca

SEQ ID NO: 264 - DNA, 125 bp, unidentified
  source 1..125: mol_type = genomic DNA; note = S. thermophiles
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 265 - DNA, 121 bp, unidentified
  source 1..121: mol_type = genomic DNA; note = S. thermophiles
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 266 - DNA, 109 bp, unidentified
  source 1..109: mol_type = genomic DNA; note = S. thermophiles
nnnnnnnnnnnnnnnnnnnngtttttgtactctcagaaatgcagaagctacaaagataaggcttcatgccgaaatcaacaccctgtcattttatggcagggtgtttttt

SEQ ID NO: 267 - DNA, 102 bp, unidentified
  source 1..102: mol_type = genomic DNA; note = S. pyogenes
nnnnnnnnnnnnnnnnnnnngttttagagctagaaatagcaagttaaaataaggctagtccgttatcaacttgaaaaagtggcaccgagtcggtgctttttt

SEQ ID NO: 268 - DNA, 87 bp, unidentified
  source 1..87: mol_type = genomic DNA; note = S. pyogenes
nnnnnnnnnnnnnnnnnnngttttagagctagaaatagcaagttaaaataaggctagtccgttatcaacttgaaaaagtgttttttt

SEQ ID NO: 269 - DNA, 76 bp, unidentified
  source 1..76: mol_type = genomic DNA; note = S. pyogenes
nnnnnnnnnnnnnnnnnnnngttttagagctagaaatagcaagttaaaataaggctagtccgttatcatttttttt

SEQ ID NO: 270 - DNA, 32 bp, Synthetic construct
  source 1..32: mol_type = other DNA
taatggaaacttcctcatgaaaaagtctttag

SEQ ID NO: 271 - DNA, 30 bp, Synthetic construct
  source 1..30: mol_type = other DNA
acagagagaataacataaaaacagggaagc

SEQ ID NO: 272 - protein, 84 aa, synthetic construct
MTNLSDIIEKETGKQLVIQESILMLPEEVEEVIGNKPESDILVHTAYDESTDENVMLLTSDAPEYKPWALVIQDSNGENKIKML

SEQ ID NO: 273 - DNA, 253 bp, Synthetic construct
  source 1..253: mol_type = other DNA
tttgcttatccagaaaagggagtgattgcttccaggggcctcaggggaataaatcatagaatcctggacaaggtttgaaggacaggtaggatttgggtgggtggaggagggtgcatggggtcagaattgtaaccgaaaactcattccaggtggatagagaaaatttctagtgttgttgtttttaaactatttgggggactggcacagaccctttttgaatacctgatgggctcacatttctgtcgaatcccag

SEQ ID NO: 274 - DNA, 121 bp, unidentified
  source 1..121: mol_type = genomic DNA; note = N. meningitis
gttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 275 - DNA, 57 bp, Synthetic construct
  source 1..57: mol_type = other DNA
acactctttccctacacgacgctcttccgatctnnnntgcagtctcctgcttctctg

SEQ ID NO: 276 - protein, 16 aa, Synthetic construct
KRPAATKKAGQAKKKK

SEQ ID NO: 277 - protein, 17 aa, Synthetic construct
KKTELQTTNAENKTKKL

SEQ ID NO: 278 - protein, 19 aa, Synthetic construct
KRGINDRNFWRGENGRKTR

SEQ ID NO: 279 - protein, 16 aa, Synthetic construct
RKSGKIAAIVVKRPRK

SEQ ID NO: 280 - DNA, 58 bp, Synthetic construct
  source 1..58: mol_type = other DNA
acactctttccctacacgacgctcttccgatctnnnnggacatttccaccgcaaaatg

SEQ ID NO: 281 - DNA, 57 bp, Synthetic construct
  source 1..57: mol_type = other DNA
acactctttccctacacgacgctcttccgatctnnnnctgcacctagcctccatgtc

SEQ ID NO: 282 - DNA, 56 bp, Synthetic construct
  source 1..56: mol_type = other DNA
acactctttccctacacgacgctcttccgatctnnnnggaggtggagagaggatgt

SEQ ID NO: 283 - DNA, 48 bp, Synthetic construct
  source 1..48: mol_type = other DNA
tggagttcagacgtgtgctcttccgattggtggagtgctctgtgtttg

SEQ ID NO: 284 - DNA, 47 bp, Synthetic construct
  source 1..47: mol_type = other DNA
tggagttcagacgtgtgctcttccgatgctacagaaaggtcagcagc

SEQ ID NO: 285 - DNA, 49 bp, Synthetic construct
  source 1..49: mol_type = other DNA
tggagttcagacgtgtgctcttccgatctgctgtggcatccagagacat

SEQ ID NO: 286 - DNA, 49 bp, Synthetic construct
  source 1..49: mol_type = other DNA
tggagttcagacgtgtgctcttccgatcttcctgaggtctaggaacccg

SEQ ID NO: 287 - DNA, 54 bp, Synthetic construct
  source 1..54: mol_type = other DNA
tggagttcagacgtgtgctcttccgatctgcaccaggtggtaacgagctgcatc

SEQ ID NO: 288 - DNA, 217 bp, Synthetic construct
  source 1..217: mol_type = other DNA
ttggctttccaaatcagtgggtctgacttgaggtctgtgatgtgacccttttcctcacctgctcaaccattattcacatggactccatcatattcatttgtagtcattcccagagtggcccagtgagggtctcgctgtatgagagtcggctacggaatttaggagaaacagaagtttcttggctttcatgctgagcttgttggtctaagcttatgag

SEQ ID NO: 289 - DNA, 225 bp, Synthetic construct
  source 1..225: mol_type = other DNA
tttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgg

SEQ ID NO: 290 - DNA, 244 bp, Synthetic construct
  source 1..244: mol_type = other DNA
ctggttgaccaatctgtggtgaatagtggaaatctgctcaatgacatgactcctcctgtaaatccttcacgtgaaattgaggacccagaagaccggaagcccgaggattgggatgaaagaccaaaaatcccagatccagaagctgtcaagccagatgactgggatgaagatgcccctgctaagattccagatgaagaggccacaaaacccgaaggctggttagatgatgagcctgagtacgtac

SEQ ID NO: 291 - DNA, 21 bp, Synthetic construct
  source 1..21: mol_type = other DNA
gcaaggcccggcgcacggtgg

SEQ ID NO: 292 - DNA, 20 bp, Synthetic construct
  source 1..20: mol_type = other DNA
acgtgctcagtctgggcccc

SEQ ID NO: 293 - DNA, 20 bp, Synthetic construct
  source 1..20: mol_type = other DNA
gcgaggcagagggtccaaag

SEQ ID NO: 294 - DNA, 20 bp, Synthetic construct
  source 1..20: mol_type = other DNA
ccactgtagtcacacagcac

SEQ ID NO: 295 - DNA, 59 bp, Synthetic construct
  source 1..59: mol_type = other DNA
acactctttccctacacgacgctcttccgatctnnnntggctttccaaatcagtgggtc

SEQ ID NO: 296 - DNA, 60 bp, Synthetic construct
  source 1..60: mol_type = other DNA
acactctttccctacacgacgctcttccgatctnnnnatttgatggagttggacatggcc

SEQ ID NO: 297 - DNA, 58 bp, Synthetic construct
  source 1..58: mol_type = other DNA
acactctttccctacacgacgctcttccgatctnnnnctggttgaccaatctgtggtg

SEQ ID NO: 298 - DNA, 52 bp, Synthetic construct
  source 1..52: mol_type = other DNA
tggagttcagacgtgtgctcttccgatctctcataagcttagaccaacaagc

SEQ ID NO: 299 - DNA, 313 bp, Synthetic construct
  source 1..313: mol_type = other DNA
gatcgcttttccgagcttctggcggtctcaagcactacctacgtcagcacctgggaccccgccaccgtgcgccgggccttgcagtgggcgcgctacctgcgccacatccatcggcgctttggtcggcatggccccattcgcacggctctggagcggcggctgcacaaccagtggaggcaagagggcggctttgggcggggtccagttccgggattagcgaacttccaggccctcggtcactgtgacgtcctgctctctctgcgcctgctggagaaccgggccctcggggatgcagctcgttaccacctggtgc

SEQ ID NO: 300 - DNA, 46 bp, Synthetic construct
  source 1..46: mol_type = other DNA
tggagttcagacgtgtgctctccagctacttgttcttgagtgaagg

SEQ ID NO: 301 - DNA, 41 bp, Synthetic construct
  source 1..41: mol_type = other DNA
tggagttcagacgtgtgctctctgcgtctggatcaggtacg

SEQ ID NO: 302 - DNA, 34 bp, Synthetic construct
  source 1..34: mol_type = other DNA
tcgagctcggtacctaatacgactcactataagg

SEQ ID NO: 303 - DNA, 148 bp, Synthetic construct
  source 1..148: mol_type = other DNA
tttttttttttttttttttttttttttttttttttttttttttttttttttttttttttttttttttttttttttttttttttttttttttttttttttttttttttttttttttttttcttcctactcaggctttattcaaagacca

SEQ ID NO: 304 - DNA, 60 bp, Synthetic construct
  source 1..60: mol_type = other DNA
acactctttccctacacgacgctcttccgatctnnnngatcgcttttccgagcttctggc

SEQ ID NO: 305 - DNA, 48 bp, Synthetic construct
  source 1..48: mol_type = other DNA
aattcacctcgaaagcaagttgataaactgatacaattaaaggctcct

SEQ ID NO: 306 - DNA, 11 bp, Synthetic construct
  source 1..11: mol_type = other DNA
aaggaggaaaa

SEQ ID NO: 307 - DNA, 176 bp, Synthetic construct
  source 1..176: mol_type = other DNA
cacagctaacaccacgtcgtccctatctgctgccctaggtctatgagtggttgctggataactttacgggcatgcataaggctcgtatgatatattcagggagaccacaacggtttccctctacaaataattttgtttaacttttactagagtgggaccctacctgcaggtgcagt

SEQ ID NO: 308 - DNA, 15 bp, Synthetic construct
  source 1..15: mol_type = other DNA
aaggaggaaaaaaaa

SEQ ID NO: 309 - DNA, 39 bp, Synthetic construct
  source 1..39: mol_type = other DNA
ggctttacactttatgcttccggctcgtatgttgtgtgg

SEQ ID NO: 310 - DNA, 23 bp, Synthetic construct
  source 1..23: mol_type = other DNA
taatacgactcactatagggaga

SEQ ID NO: 311 - DNA, 13 bp, Synthetic construct
  source 1..13: mol_type = other DNA
aaggaaaaaaaaa

SEQ ID NO: 312 - DNA, 176 bp, Synthetic construct
  source 1..176: mol_type = other DNA
cacagctaacaccacgtcgtccctatctgctgccctaggtctatgagtggttgctggataactttacgggcatgcataaggctcgtaggctatattcagggagaccacaacggtttccctctacaaataattttgtttaacttttactagagtgggaccctacctgcaggtgcagt

SEQ ID NO: 313 - DNA, 176 bp, Synthetic construct
  source 1..176: mol_type = other DNA
cacagctaacaccacgtcgtccctatctgctgccctaggtctatgagtggttgctggataactttacgggcatgcataaggctcgtaatatatattcagggagaccacaacggtttccctctacaaataattttgtttaacttttactagagtgggaccctacctgcaggtgcagt

SEQ ID NO: 314 - DNA, 176 bp, Synthetic construct
  source 1..176: mol_type = other DNA
cacagctaacaccacgtcgtccctatctgctgccctaggtctatgagtggttgctggataactttacgggcatgcataaggctcgtataatatattcagggagaccacaacggtttccctctacaaataattttgtttaacttttactagagtgggaccctacctgcaggtgcagt

SEQ ID NO: 315 - protein, 167 aa, unidentified
  source 1..167: note = E. coli
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 316 - DNA, 15 bp, Synthetic construct
  source 1..15: mol_type = other DNA
actacatcatcaggc

SEQ ID NO: 317 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 318 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 319 - protein, 183 aa, unidentified
  source 1..183: note = Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 320 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 321 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 322 - protein, 161 aa, unidentified
  source 1..161: note = Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 323 - protein, 178 aa, Synthetic construct
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGAAGSLYDLSADPRLNHQVRLSPGVCQEECGTMLSDFFRDLRRRKKAKATPALFIDERKVPPEP

SEQ ID NO: 324 - DNA, 57 bp, Synthetic construct
  source 1..57: mol_type = other DNA
aatgatacggcgaccaccgagatctacacattactcgacactctttccctacacgac

SEQ ID NO: 325 - protein, 178 aa, unidentified
  source 1..178: note = E. coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 326 - protein, 166 aa, Synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 327 - DNA, 57 bp, Synthetic construct
  source 1..57: mol_type = other DNA
aatgatacggcgaccaccgagatctacactccggagaacactctttccctacacgac

SEQ ID NO: 328 - DNA, 57 bp, Synthetic construct
  source 1..57: mol_type = other DNA
aatgatacggcgaccaccgagatctacaccgctcattacactctttccctacacgac

SEQ ID NO: 329 - DNA, 57 bp, Synthetic construct
  source 1..57: mol_type = other DNA
aatgatacggcgaccaccgagatctacacgagattccacactctttccctacacgac

SEQ ID NO: 330 - DNA, 54 bp, Synthetic construct
  source 1..54: mol_type = other DNA
gtgactggagttcagacgtgtgctcttccgatctgtggaaaggacgaaacaccg

SEQ ID NO: 331 - DNA, 29 bp, Synthetic construct
  source 1..29: mol_type = other DNA
aatgatacggcgaccaccgagatctacac

SEQ ID NO: 332 - DNA, 56 bp, Synthetic construct
  source 1..56: mol_type = other DNA
caagcagaagacggcatacgagatatcgtgatgtgactggagttcagacgtgtgct

SEQ ID NO: 333 - DNA, 56 bp, Synthetic construct
  source 1..56: mol_type = other DNA
caagcagaagacggcatacgagatatacatcggtgactggagttcagacgtgtgct

SEQ ID NO: 334 - DNA, 56 bp, Synthetic construct
  source 1..56: mol_type = other DNA
caagcagaagacggcatacgagatatgcctaagtgactggagttcagacgtgtgct

SEQ ID NO: 335 - DNA, 56 bp, Synthetic construct
  source 1..56: mol_type = other DNA
caagcagaagacggcatacgagatattggtcagtgactggagttcagacgtgtgct

SEQ ID NO: 336 - DNA, 56 bp, Synthetic construct
  source 1..56: mol_type = other DNA
caagcagaagacggcatacgagatatcactgtgtgactggagttcagacgtgtgct

SEQ ID NO: 337 - DNA, 56 bp, Synthetic construct
  source 1..56: mol_type = other DNA
caagcagaagacggcatacgagatatattggcgtgactggagttcagacgtgtgct

SEQ ID NO: 338 - DNA, 56 bp, Synthetic construct
  source 1..56: mol_type = other DNA
caagcagaagacggcatacgagatatgatctggtgactggagttcagacgtgtgct

SEQ ID NO: 339 - RNA, 91 nt, unidentified; identical to SEQ ID NO: 78
  source 1..91: mol_type = genomic RNA; note = S. pyogenes

SEQ ID NO: 340 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 341 - protein, 1087 aa, Synthetic construct
AAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDAALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVSEQIDNO

SEQ ID NO: 342 - protein, 1060 aa, Synthetic construct
GKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGSEQIDNO

SEQ ID NO: 343 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 344 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIAMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 345 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHAAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 346 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHAAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 347 - protein, 1053 aa, Staphylococcus aureus
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 348 - protein, 984 aa, Synthetic construct
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 349 - protein, 1082 aa, Synthetic construct
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 350 - protein, 166 aa, unidentified
  source 1..166: note = E. coli
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 351 - protein, 1053 aa, Staphylococcus aureus
MGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 352 - protein, 1082 aa, Neisseria meningitidis
MAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 353 - protein, 1082 aa, Synthetic construct
MAAFKSNPINYILGLDIGIASVGWAMVEIDEEGNPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIAKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPSSKKQRILLQKFDEDGFKECNLNDTRYVNRFLCQFVADHILLTGKGKRRVVASNGQITNLLRGFWRLRKVRAENDRHHALDAVVVACSTVAMQQKITRFVRYKEMNAFDGKTVDKETGKVLYQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVR

SEQ ID NO: 354 - protein, 171 aa, Streptococcus pyogenes
MPYSLEEQTYFMQEALKEAEKSLQKAEIPIGCVIVKDGEIIGRGHNAREESNQAIMHAEIMAINEANAHEGNWRLLDTTLFVTIEPCVMCSGAIGLARIPHVIYGASNQKFGGADSLYQILTDERLNHRVQVERGLLAADCANIMQTFFRQGRERKKIAKHLIKEQSDPFD

SEQ ID NO: 355 - protein, 151 aa, unidentified
  source 1..151: note = Aquifex aeolicus
MGKEYFLKVALREAKRAFEKGEVPVGAIIVKEGEIISKAHNSVEELKDPTAHAEMLAIKEACRRLNTKYLEGCELYVTLEPCIMCSYALVLSRIEKVIFSALDKKHGGVVSVFNILDEPTLNHRVKWEYYPLEEASELLSEFFKKLRNNII

SEQ ID NO: 356 - protein, 1368 aa, Synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 357 - protein, 1054 aa, Staphylococcus aureus
MGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 358 - protein, 1061 aa, Synthetic construct
MQENQQKQNYILGLAIGITSVGYGLIDSKTREVIDAGVRLFPEADSENNSNRRSKRGARRLKRRRIHRLNRVKDLLADYQMIDLNNVPKSTDPYTIRVKGLREPLTKEEFAIALLHIAKRRGLHNISVSMGDEEQDNELSTKQQLQKNAQQLQDKYVCELQLERLTNINKVRGEKNRFKTEDFVKEVKQLCETQRQYHNIDDQFIQQYIDLVSTRREYFEGPGNGSPYGWDGDLLKWYEKLMGRCTYFPEELRSVKYAYSADLFNALNDLNNLVVTRDDNPKLEYYEKYHIIENVFKQKKNPTLKQIAKEIGVQDYDIRGYRITKSGKPQFTSFKLYHDLKNIFEQAKYLEDVEMLDEIAKILTIYQDEISIKKALDQLPELLTESEKSQIAQLTGYTGTHRLSLKCIHIVIDELWESPENQMEIFTRLNLKPKKVEMSEIDSIPTTLVDEFILSPVVKRAFIQSIKVINAVINRFGLPEDIIIELAREKNSKDRRKFINKLQKQNEATRKKIEQLLAKYGNTNAKYMIEKIKLHDMQEGKCLYSLEAIPLEDLLSNPTHYEVDHIIPRSVSFDNSLNNKVLVKQSENSKKGNRTPYQYLSSNESKISYNQFKQHILNLSKAKDRISKKKRDMLLEERDINKFEVQKEFINRNLVDTRYATRELSNLLKTYFSTHDYAVKVKTINGGFTNHLRKVWDFKKHRNHGYKHHAEDALVIANADFLFKTHKALRRTDKILEQPGLEVNDTTVKVDTEEKYQELFETPKQVKNIKQFRDFKYSHRVDKKPNRQLINDTLYSTREIDGETYVVQTLKDLYAKDNEKVKKLFTERPQKILMYQHDPKTFEKLMTILNQYAEAKNPLAAYYEDKGEYVTKYAKKGNGPAIHKIKYIDKKLGSYLDVSNKYPETQNKLVKLSLKSFRFDIYKCEQGYKMVSIGYLDVLKKDNYYYIPKDKYEAEKQKKKIKESDLFVGSFYYNDLIMYEDELFRVIGVNSDINNLVELNMVDITYKDFCEVNNVTGEKRIKKTIGKRVVLIEKYTTDILGNLYKTPLPKKPQLIFKRGEL

SEQ ID NO: 359 - protein, 167 aa, Synthetic construct
MSEVEFSHEYWMRHALTLAKRARDEGEAPVGAVLVLNNRVIGEGWIRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 360 - protein, 167 aa, Synthetic construct
MSEVEFSHEYWMRHALTLAKRARDEGEAPVGAVLVLNNRVIGEGWNRRIGLHDPTAHAEIMALRQGGLVMSNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 361 - protein, 167 aa, Synthetic construct
MSEVEFSHEYWMRHALTLAKRARDEGEAPVGAVLVLNNRVIGEGWLRRIGLHDPTAHAEIMALRQGGLVMQNPRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 362 - protein, 167 aa, Synthetic construct; identical to SEQ ID NO: 47

SEQ ID NO: 363 - protein, 167 aa, Synthetic construct
MSEVEFSHEYWMRHALTLAKRARDEGEAPVGAVLVLNNRVIGEGWNRAIGLHDPTAHAEIMALRQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 364 - protein, 167 aa, Synthetic construct; identical to SEQ ID NO: 45

SEQ ID NO: 365 - protein, 167 aa, Synthetic construct; identical to SEQ ID NO: 50

SEQ ID NO: 366 - protein, 167 aa, Synthetic construct
MSEVEFSHEYWMRHALTLAKRARDEGEVPVGAVLVLNNRVIGEGWLRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 367 - protein, 167 aa, Synthetic construct; identical to SEQ ID NO: 42

SEQ ID NO: 368 - protein, 167 aa, Synthetic construct; identical to SEQ ID NO: 46

SEQ ID NO: 369 - protein, 167 aa, Synthetic construct
MSEVEFSHEYWMRHALTLAKRARDEGEAPVGAVLVLNNRVIGEGWNRPIGLHDPTAHAEIMALRQGGLVMQNHRLIDAPLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 370 - protein, 167 aa, Synthetic construct
MSEVEFSHEYWMRHALTLAKRARDEGEVPVGAVLVLNNRVIGEGWIRAIGLHDPTAHAEIMALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 371 - protein, 167 aa, Synthetic construct; identical to SEQ ID NO: 50

SEQ ID NO: 372 - protein, 167 aa, Synthetic construct; identical to SEQ ID NO: 52

SEQ ID NO: 373 - protein, 167 aa, Synthetic construct
MSEVEFSHEYWMRHALTLAKRARDEGEAPVGAVLVLNNRVIGEGWCRRIGLHDPTAHAEIMALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKVQSSIN

SEQ ID NO: 374 - protein, 167 aa, Synthetic construct; identical to SEQ ID NO: 51

SEQ ID NO: 375 - protein, 167 aa, Synthetic construct; identical to SEQ ID NO: 53

SEQ ID NO: 376 - protein, 167 aa, Synthetic construct; identical to SEQ ID NO: 54

SEQ ID NO: 377 - protein, 167 aa, Synthetic construct; identical to SEQ ID NO: 50

SEQ ID NO: 378 - protein, 167 aa, Synthetic construct; identical to SEQ ID NO: 45

SEQ ID NO: 379 - protein, 167 aa, Synthetic construct
MSEVEFSHEYWMRHALTLAKRARDEGEAPVGAVLVLNNRVIGEGWVRPIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 380 - protein, 167 aa, Synthetic construct; identical to SEQ ID NO: 53

SEQ ID NO: 381 - protein, 167 aa, Synthetic construct
MSEVEFSHEYWMRHALTLAKRARDEGEAPVGAVMVLNNRVIGEGWLRRIGLHDPTAHAEIMALRQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 382 - protein, 167 aa, Synthetic construct; identical to SEQ ID NO: 47

SEQ ID NO: 383 - protein, 167 aa, Synthetic construct
MSEVEFSHEYWMRHALTLAKRARDEGEAPVGAVLVLNNRVIGEGWLRPIGLHDPTAHAEIMALKQGGLVMQNPRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 384 - protein, 4 aa, synthetic construct
EMLA

SEQ ID NO: 385 - DNA, 29 bp, synthetic construct
  source 1..29: mol_type = other DNA
gcaagagcacaagaggaagagagagaccc

SEQ ID NO: 386 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD